SEQUENCE LISTING (7 sequences)

SEQ ID NO: 1 - protein, 710 aa, Homo sapiens
TGMLVVNGVDLLSSRSDQNRREHHTDEYEYDELIVRRGQPFHMLLLLSRTYESSDRITLELLIGNNPEVGKGTHVIIPVGKGTGGSGGWKAQVVKASGQNLNLRVHTSPNAIIGKFQFTVRTQSDAGEFQLPFDPRNEIYILFNPWCPEDIVYVDHEDWRQEYVLNESGTGRIYYGTEAQIGERTWNYGQFDHGVLDACLYILDRRGMPYGGRGDPVNVSRVISAMVNSLDDNGVLIGNWSGDYSRGTNTGPSAWVGSVEILLSYLRTGYSVPYGQCWVFAGVTTTVLRCLGLATRTVTNFNSAHDTDTSLTMDIYFDENMKPLEHLNHDSVWNFTGHVWNDCWMKRPDLPSGFDGWQVVDATPQETSSGIFCCGPCSVESIKNGLVYMKYDTPFIFAEVNSDKVYWQRQDDGSFKIVYTGVEEKAIGTLIVTKAISSNMREDITYLYKHPEGSDAERKAVETAAAHGSKPNVYANRGSAETGDVAMQVEAQDAVMGQDLMVSVMLINHSSSRRTVKLHLYLSVTFYTGVSGTIFKETKKEVELAPGASDRVTMPVAYKEYRTGPHLVDQGAMLLNVSGHVKESGQVLAKQHTFRLRTPDLSLTLLGAAVVGQECEVQIVFKNPLPVTLTNVVFRLEGSGLQRPKILNVTGGDIGGNETVTLRQSFVPVRPGPRQLIASLDSPQLSQVHGVIQVDVAPAPGDGGFFSDAG

SEQ ID NO: 2 - protein, 705 aa, Homo sapiens
TGMAEELVLERCDLELETNGRDHHTADLCREKLVVRRGQPFWLTLHFEGRNYEASVDSLTFSVVTGPAPSQEAGTKARFPLRDAVTGEEGDWTATVVDQQDCTLSLQLTTPANAPIGLYRLSLEASTGYQGSSFVLGHFILLFNAWCPADAVYLDSEEERQEYVLTQQGTGFIYQGSAKFIKNIPWNFGQFEDGILDICLILLDVNPKFLKNAGRDCSRRSSPVYVGRVVSGMVNCNDDQGVLLGRWDNNYGDGVSTGPMSWIGSVDILRRWKNHGCQRVKYGQCWVFAAVACTVLRCLGIPTRVVTNYNSAHDQNSNLLIEYFRNEFGEIQGDKSEMIWNFTGHCWVESWMTRPDLQPGYEGWQALDPTPQEKSEGTYCCGPVPVRAIKEGDLSTKYDAPFVFAEVNADVVDWIQQDDGSVHKSITGNRSLIVGLKISTKSVGRDEREDITHTYKYPEGSSEEREAFTRANHLNKLAEKEETGTGMAMRIRVGQSMNMGSDFDVFAHITNNTAEEYVCRLLLCARTVSYNGILGPECGTKYLLNLNLEPFSEKSVPLCILYEKYRTGDCLTESNLIKVRALLVEPVINSYLLAERDLYLENPEIKIRILGEPKQKRKLVAEVSLQNPLPVALEGCTFTVEGAGLTEEQKTVETGIPDPVEAGEEVKVRMDLLPLHMGLHKLVVNFESDKLKAVKGFRNVIIGPA

SEQ ID NO: 3 - protein, 710 aa, Homo sapiens
TGMAALGVQSINWQKAFNRQAHHTDKFSSQELILRRGQFQVLMIMNKGLGSNERLEFIVSTGPYPSESAMTKAVFPLSNGTGSSGGWSAVLQASNGNTLTISISSPASAPIGRYTMALQIFSQGGISSVKLGTFILLFNPWLNVDSVFMGNHAEREEYVQEDAGTGIIFVGSTNRIGMIGWNFGQFEEDILSICLSILDRSLNFRRDAATDVASRNDPKYVGRVLSAMINSNDDNGVLAGNWSGTYTGGRDTGPRSWNGSVEILKNWKKSGFSPVRYGQCWVFAGTLNTALRSLGIPSRVITNFNSAHDTDRNLSVDVYYDPMGNPLDKGSDSVWNFTGHVWNEGWFVRSDLGPSYGGWQVLDATPQERSQGVFQCGPASVIGVREGDVQLNFDMPFIFAEVNADRITWLYDNTTGKQWKNSTGVNSHTIGRYISTKAVGSNARMDVTDKYKYPEGSDQERQVFQKALGKLKPNTPFAATSSMGLETEEQEPSTGIIGKLKVAGMLAVGKEVNLVLLLKNLSRDTKTVTVNMTAWTIIYNGTLVHEVWKDSATMSLDPEEEAEHPIKISYAQYETGKYLKSDNMIRITAVCKVPDESEVVVERDIILDNPTLTLEVLNEARVRKPVNVQMLFSNPLDEPVRDCVLMVEGSGLLLGNLKIDTGVPTLGPKEGSRVRFDILPSRSGTKQLLADFSCNKFPAIKAMLSIDVAE

SEQ ID NO: 4 - protein, 652 aa, Homo sapiens
TGELQVLHIDFLNQDNAVSHHTWEFQTSSPVFRRGQVFHLRLVLNQPLQSYHQLKLEFSTGPNPSIAKHTLVVLDPRTPSTGDHYNWQATLQNESGKEVTVAVTSSPNAILGKYQLNVKTGNHILKSEENILYLLFNPWCKEDMVFMPDEDERKEYILNDTGTGCHYVGAARSIKCKPWNFGQFEKNVLDCCISLLTESSLKPTDRRDPVLVCRAMCAMMSFEKGQGVLIGNWTGDYEGGTATGPYKWTGSAPILQQYYNTKQAVCFGQCWVFAGILTTVLRALGIPARSVTGFDSAHDTERNLTVDTYVNENGEKITSMTHDSVWNFTGHVWTDAWMKRPDLPKGYDGWQAVDATPQERSQGVFCCGPSPLTAIRKGDIFIVYDTRFVPSEVNGDRLIWLVKMVNGQEELHVISTGMETTSIGKNISTKAVGQDRRRDITYEYKYPEGSSEERQVMDHAFLLLSSEREHRRPVKENFTGLHMSVQSDDVLLGNSVNFTVILKRKTAALQNVNILGSFELQLYTGKKMAKLCDLNKTSQIQGQVSEVTLTLDSKTYINSLTGAILDDEPVIRGFIIAEIVESKEIMASEVFTSFQYPEFSIELPNTGRIGQLLVCNCIFKNTLAIPLTDVKFSLESLGISSLQTSDHTGGTV

SEQ ID NO: 5 - protein, 659 aa, Homo sapiens
TGMAQGLEVALTDLQSSRNNVRHHTEEITVDHLLVRRGQAFNLTLYFRNRSFQPGLDNIIFVVETGPLSDLALGTRAVFSLARHHTGSPSPWIAWLETNGATSTEVSLCAPPTAAVGRYLLKIHIDSFQGSVTAYQLGEFILLFNPWCPEDAVYLDSEPQRQEYVMNDYGTGFIYQGSKNWIRPCPWNYGQFEDKIIDICLKLLDKSLHFQTDPATDCALRGSPVYVSRVVCAMINSNDDNGVLNGNWSENYTDGANTGPAEWTGSVAILKQWNATGCQPVRYGQCWVFAAVMCTVMRCLGIPTRVITNFDSGHDTDGNLIIDEYYDNTGRILGNKKKDTIWNFTGHVWNECWMARKDLPPAYGGWQVLDATPQEMSNGVYCCGPASVRAIKEGEVDLNYDTPFVFSMVNADCMSWLVQGGKEQKLHTGQDTSSVGNFISTKSIQSDERDDITENYKYEEGSLQERQVFLKALQKLKARSFHGSQRGAELQPSRPTSLSQDSPRSLHTPSLRPSTGDVVQVSLKFKLLDPPNMGQDICFVLLALNMSSQFKDLKVNLSAQSLLHDGSPLSPFWQDTAFITLSPKEAKTYPCKISYSQYSTGQYLSTDKLIRISALGEEKSSPEKILVNKIITLSYPSITINVLGAAVVNQPLSIQVITG

SEQ ID NO: 6 - protein, 706 aa, Homo sapiens
FAFLNVTSVHLFKERWDTNKVDHHTDKYENNKLIVRRGQSFYVQIDFSRPYDPRRDLFRVEYVIGRYPQENKGTYIPVPIVSELFAQSGKWGAKIVMREDRSVRLSIQSSPKCIVGKFRMYVAVWTPYGVLRTSRNPETDTYILFNPWCEDDAVYLDNEKEREEYVLNDIGFAVIFYGEVNDIKTRSWSYGQFEDGILDTCLYVMDRAQMDLSGRGNPIKVSRVGSAMVNAKDDEGVLVGSWDNIYAYGVPFAPSAWTGSVDILLEYRSSENPVRYGQCWVFAGVFNTFLRCLGIPARIVTNYFSAHDNDANLQMDIFLEEDGNVNSKLTKDSVWNYFAHCWNEAWMTRPDLPVGFGGWQAVDSTPQENSDGMYRCGPASVQAIKHGHVCFQFDAPFVFAEVNSDLIYITAKKDGTHVVENFAVDATHIGKLIVTKQIGGDGMMDITDTYKFQEGQEEERLALETALMYGAKKPLNTEGVMKSRSFANVDMDFEVENAVLGKDFKLSITFRNNSHNRYTITAYLSANITFYTGVPKAEFKKETFDVTLEPLSFKKEAVLIQAGEYMFAGQLLEQASLHFFVTARINETRDVLAKQKSTVLTIPEIIIKVRGTQVVGSDMTVTVQFTNPLKETLRNVWVHLDGPGVTRPMKKMFFAREIRPNSTVQWEEVCRPWVSGHRKLIASMSSDSLRHVYGELDVQIQRRPSM

SEQ ID NO: 7 - protein, 699 aa, Homo sapiens
BMDALGIKSCDFQAARNNEEHHTKALSSRRLFVRRGQPFTIILYFRAPVRAFLPALKKVALTAQTGEQPSKINRTQATFPISSLGBDRKWWSAVVEERDAQSWTISVTTPADAVIGHYSLLLQVSGRKQLLLGQFTLLFNPWNREDAVFLKNEAQRMEYLLNQNGBLIYLGTADCIQAESWDFGQFEGDVIDLSLRLLSKDKQVEKWSQPVHVARVLGALLHFLKEQRVLPTPQTQATQEGALBLNKRRGSVPILRQWLTGRGRPVYDGQAWVLAAVACTVLRCLGIPARVVTTFASAQGTGGRLLIDEYYNEEGLQNGEGQRGRIWIFBQTSTECWMTRPALPQGYDGWQILHPSAPNGGGVLGSCDLVPVRAVKEGTLGLTPAVSDLFAAINASCVVWKCCEDGTLELTDBSNTKYVGNNISTKGVGSDRCEDITQNYKYPEGSLQEKEVLERVEKEKMEREKDNGIRPPSLETABSPLYLLLKAPSSLPLRGDAQISVTLVNHSEQEKAVQLAIGVQAVHYNGVLAAKLWRKKLHLTLSANLEKIITIGLFFSNFEBRNPPENTFLRLTAMATHSESNLSCFAQEDIAICRPHLAIKMPEKAEQYQPLTASVSLQNSLDAPMEDCVISILGRGLIHRERSYRBFRSVWPENTMCAKFQFTPTHVGLQRLTVEVDCNMFQNLTNYKSVTVVAPELSA

(19) United States
(12) Patent Application Publication
(10) Pub. No.: US 2004/0259176 A1
(43) Pub. Date: Dec. 23, 2004

(54) Structural basis for the guanine nucleotide-binding activity of tissue transglutaminase and its regulation of transamidation activity

(76) Inventors: Richard A. Cerione, Ithaca, NY (US); Jon Clardy, Boston, MA (US); Shenping Liu, Portage, MI (US)

Correspondence Address: Michael L. Goldman, Nixon Peabody LLP, Clinton Square, P.O. Box 31051, Rochester, NY, 14603-1051, US

(21) Appl. No.: 10/785,823

(22) Filed: Feb. 24, 2004

Related U.S. Application Data
(60) Provisional application No. 60/449,959, filed on Feb. 25, 2003.

(51) Int. Cl. G01N 33/574; A61K 31/00

(52) U.S. Cl. 435/7.23; 514/1

(57) ABSTRACT

The present invention relates to methods of facilitating death of cancer cells in a subject by inhibiting tissue transglutaminase in the subject under conditions effective to facilitate death of cancer cells. Also disclosed are methods for identifying candidate compounds suitable for facilitating death of cancer cells in a subject by contacting tissue transglutaminase with a compound and identifying those compounds which bind to the tissue transglutaminase as candidate compounds suitable for facilitating death of cancer cells in a subject. The present invention also discloses methods of producing a tissue transglutaminase crystal suitable for X-ray diffraction as well as crystals produced by such methods. Also disclosed are compounds suitable for facilitating death of cancer cells in a subject as well as methods for designing such compounds.

This application claims the benefit of U.S. Provisional Patent Application Ser. No. 60/449,959, filed Feb. 25, 2003, which is hereby incorporated by reference in its entirety.

This invention arose out of research sponsored by the National Institutes of Health (Grant Nos. GM61762 and CA59021). The U.S. Government may have certain rights in this invention.

### FIELD OF THE INVENTION

The present invention relates to facilitating death of cancer cells in a subject by inhibiting tissue transglutaminase in the subject. Compounds suitable for facilitating death of cancer cells in a subject as well as methods for designing such compounds and methods for identifying candidate compounds are also disclosed. Also disclosed is a method of producing a tissue transglutaminase crystal suitable for X-ray diffraction.

### BACKGROUND OF THE INVENTION

Tissue transglutaminase (TG, also called type II transglutaminase) catalyzes the Ca²⁺-dependent formation of a new amide bond between the γ-carboxamide of glutamine and the ε-amino group of lysine or another primary amine (Folk, “Transglutaminases,” Annu. Rev. Biochem., 49:517-531 (1980)) (see scheme below).

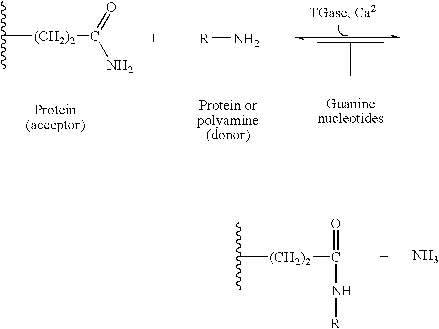

TG activity, which is found in the cytosol, plasma membrane, and nucleus of cells, has been implicated in a variety of physiological activities and pathological processes, including neuronal growth and regeneration (Mahoney et al., “Stabilization of Neurites in Cerebellar Granule Cells by Transglutaminase Activity: Identification of Midkine and Galectin-3 as Substrates,” Neuroscience, 101:141-155 (2000); Eitan et al., “Recovery of Visual Response of Injured Adult Rat Optic Nerves Treated with Transglutaminase,” Science, 26:1764-1768 (1994); Eitan et al., “A Transglutaminase That Converts Interleukin-2 Into a Factor Cytotoxic to Oligodendrocytes,” Science, 261:106-108 (1993)), bone development (Kaartinen et al., “Cross-linking of Osteopontin by Tissue Transglutaminase Increases Its Collagen Binding Properties,” J. Biol. Chem., 274:1729-1735 (1999); Aeschlimann et al., “Tissue Transglutaminase and Factor XIII in Cartilage and Bone Remodeling,” Semin. Thromb. Hemostasis, 22:437-443 (1996)), angiogenesis (Upchurch et al., “Localization of Cellular Transglutaminase on the Extracellular Matrix After Wounding: Characteristics of the Matrix Bound Enyzme,” J. Cell. Physiol., 149:375-382 (1991)), wound healing (Upchurch et al., “Localization of Cellular Transglutaminase on the Extracellular Matrix After Wounding: Characteristics of the Matrix Bound Enyzrne,” J. Cell. Physiol., 149:375-382 (1991)), cellular differentiation, and apoptosis (Chiocca et al., “Regulation of Tissue Transglutaminase Gene Expression as a Molecular Model for Retinoid Effects on Proliferation and Differentiation,” J. Cell. Biochem., 39:293-304 (1989); Piacentini et al., “The Expression of ‘Tissue’ Transglutaminase in Two Human Cancer Cell Lines is Related With the Programmed Cell Death (Apoptosis),” Eur. J. Cell Biol., 54:246-254 (1991); Nemes et al., “Identification of Cytoplasmic Actin as an Abundant Glutaminyl Substrate for Tissue Transglutaminase in HL-60 and U937 Cells Undergoing Apoptosis,” J. Biol. Chem., 272:20577-20583 (1997)). During apoptosis, for example, TG-catalyzed crosslinking of proteins results in the irreversible formation of scaffolds that could prevent the leakage of harmful intracellular components (Melino et al., “Assays for Transglutaminases in Cell Death,” Methods Enzymol., 322:433-472 (2000)). Retinoic acid (RA)-stimulated increases in TG expression and activation accompany RA-induced cellular differentiation (Chiocca et al., “Regulation of Tissue Transglutaminase Gene-Expression as a Molecular Model for Retinoid Effects on Proliferation and Differentiation,” J. Cell. Biochem., 39:293-304 (1989); Suedhoff et al., “Differential Expression of Transglutaminase in Human Erythroleukemia Cells in Response to Retinoic Acid,” Cancer Res., 50:7830-7834 (1990)). This increased TG expression, coupled with the finding that two of the primary targets for TG, the eukaryotic initiation factor eIF-5A and the retinoblastoma gene product (Singh et al., “Identification of the Eukaryotic Initiation Factor 5A as a Retinoic Acid-Stimulated Cellular Binding Partner for Tissue Transglutaminase II,” J. Biol. Chem., 273:1946-1950 (1998); Oliverio et al., “Tissue Transglutaminase-Dependent Posttranslational Modification of the Retinoblastoma Gene Product in Promonocytic Cells Undergoing Apoptosis,” Mol. Cell. Biol., 17:6040-6048 (1997)), are essential for cell viability has led to the suggestion that TG activity is necessary for ensuring cell survival under conditions of differentiation or cellular stress. It has also been proposed that the dysregulation of TG activity may be associated with neurodegenerative conditions such as Alzheimer's disease and Huntington's disease (Green, “Human Genetic Diseases Due to Codon Reiteration: Relationship to an Evolutionary Mechanism,” Cell, 74:955-956 (1993); Lorand, “Neurodegenerative Diseases and Transglutaminase,” Proc. Natl. Acad., Sci. USA, 93:14310-14313 (1996); Lesort et al., “Tissue Transglutaminase: A Possible Role in Neurodegenerative Diseases,” Prog. Neurobiol., 61:439-463 (2000)).

TG's ability to bind and hydrolyze GTP with affinity and rates like those of traditional G proteins distinguishes it from other transglutaminases and suggests that TG, like other G proteins, participates in signaling pathways (Achyuthan et al., “Identification of a Guanosine Triphosphate-Binding Site on Guinea Pig Liver Transglutaminase. Role of GTP and Calcium Ions in Modulating Activity,” J. Biol. Chem., 262:1901-1906 (1987); Singh et al., “Identification and Biochemical Characterization of an 80 Kilodalton GTP-Binding Transglutaminase From Rabbit Liver Nuclei,” Biochemistry, 34:15863-15871 (1995); Mian et al., “The Importance of the GTP-Binding Protein Tissue Transglutaminase in the Regulation of Cell Cycle Progression,” FEBS Lett., 370:27-31 (1995); Nakaoka et al., “Gh: A GTP-Binding Protein With Transglutaminase Activity and Receptor Signaling Function,” Science, 264:1593-1596 (1994)). Among the studies implicating TG as a signal transducer in biological response pathways, the best documented is its role in α1-adrenergic receptor-mediated stimulation of phospholipase C-δ activity (Nakaoka et al., “Gh: A GTP-Binding Protein With Transglutaminase Activity and Receptor Signaling Function,” Science, 264:1593-1596 (1994); Feng et al., “Evidence That Phospholipase δ1 is the Effector in the Gₕ (Transglutaminase II)-Mediated Signaling,” J. Biol. Chem., 271:16451-16454 (1996); Hwang et al., “Interaction Site of GTP Binding G (Transglutaminase II) with Phospholipase C,” J. Biol. Chem., 270:27058-27062 (1995)). It was originally reported that an ≈70- to 80-kDa GTP-binding protein (named Gh) was responsible for coupling α1-adrenergic agonists to the stimulation of phosphoinositide lipid metabolism (Baek et al., “Evidence that the Gh Protein is a Signal Mediator From Alpha 1-Adrenoceptor to a Phospholipase C. I. Identification of Alpha 1-Adrenoceptor-coupled Gh Family and Purification of Gh7 From Bovine Heart,” J. Biol. Chem., 268:27390-27397 (1993)), and it was subsequently demonstrated that Gh was identical to TG (Nakaoka et al., “Gh: A GTP-Binding Protein With Transglutaminase Activity and Receptor Signaling Function,” Science, 264:1593-1596 (1994)). The GTP-binding/GTPase cycle of TG is closely linked to its transamidation activity, with guanine nucleotide binding having a negative regulatory effect that can be overcome by high concentrations of Ca²⁺ (Achyuthan et al., “Identification of a Guanosine Triphosphate-Binding Site on Guinea Pig Liver Transglutaminase. Role of GTP and Calcium Ions in Modulating Activity,” J. Biol. Chem., 262:1901-1906 (1987); Singh et al., “Identification and Biochemical Characterization of an 80 Kilodalton GTP-Binding Transglutaminase from Rabbit Liver Nuclei,” Biochemistry, 34:15863-15871 (1995)).

Because of the lack of sequence similarity between TG and either the large or small G proteins, the structural basis for TG's ability to bind guanine nucleotides with high affinity and hydrolyze GTP was not understood. The structural mechanism by which guanine nucleotide binding exerts such marked regulatory effects on transamidation activity is also unknown.

The present invention is directed to overcoming these deficiencies in the art.

### SUMMARY OF THE INVENTION

The present invention relates to a method of facilitating death of cancer cells in a subject. The method involves inhibiting tissue transglutaminase in the subject under conditions effective to facilitate death of cancer cells.

The present invention also relates to a method of producing a tissue transglutaminase crystal suitable for X-ray diffraction. The method first involves subjecting a solution of tissue transglutaminase under conditions effective to grow a crystal of tissue transglutaminase to a size suitable for X-ray diffraction. Then, a tissue transglutaminase crystal suitable for X-ray diffraction is obtained.

Another aspect of the present invention relates to a method for identifying candidate compounds suitable for facilitating death of cancer cells in a subject. The method first involves contacting tissue transglutaminase with a compound. Those compounds which bind to the tissue transglutaminase are identified as candidate compounds suitable for facilitating death of cancer cells in a subject.

The present invention also relates to a method for designing a compound suitable for facilitating death of cancer cells in a subject. The method first involves providing a three-dimensional structure of a crystallized tissue transglutaminase. A compound having a three-dimensional structure which will bind to one or more molecular surfaces of the tissue transglutaminase is designed.

Another aspect of the present invention relates to a compound suitable for facilitating death of cancer cells in a subject. The compound has a three-dimensional structure which will bind to one or more molecular surfaces of the tissue transglutaminase having a three dimensional crystal structure defined by the atomic coordinates set forth in FIG. 7.

The present invention also relates to a tissue transglutaminase crystal having a three dimensional crystal structure defined by the atomic coordinates set forth in FIG. 7.

Tissue transglutaminase (TG) is a Ca²⁺-dependent acyltransferase with roles in cellular differentiation, apoptosis, and other biological functions. In addition to being a transamidase, TG undergoes a GTP-binding/GTPase cycle even though it lacks any obvious sequence similarity with canonical GTP-binding (G) proteins. Guanine nucleotide binding and Ca²⁺ concentration reciprocally regulate TG's transamidation activity, with nucleotide binding being the negative regulator. The present invention reports the x-ray structure determined to 2.8-Å resolution of human TG complexed with GDP. Although the transamidation active site is similar to those of other known transglutaminases, the guanine nucleotide-binding site of TG differs markedly from other G proteins. The structure suggests a structural basis for the negative regulation of transamidation activity by bound nucleotide, and the positive regulation of transamidation by Ca²⁺.

### BRIEF DESCRIPTION OF THE DRAWINGS

The patent or application file contains at least one drawing executed in color. Copies of this patent or patent application publication with color drawings will be provided by the U.S. Patent and Trademark Office upon request and payment of the necessary fee.

FIG. 1 shows the overall structure of a human tissue transglutaminase (TG) dimer with bound GDP. TG is shown in ribbon drawing with the β-sandwich domain, the catalytic core domain, and the first and second β-barrel domain shown in green, red, cyan, and yellow, respectively. The loops connecting the first P-barrel domain to the catalytic core and the second β-barrel are shown in purple. GDP is shown as a ball-and-stick model between the catalytic core and the first α-barrel. The picture was prepared with MOLSCRIPT (Kraulis, “Molscript—A Program to Produce Both Detailed and Schematic Plots of Protein Structures,” J. Appl. Crystallogr., 24:946-950 (1991), which is hereby incorporated by reference in its entirety) and RASTER3D (Merritt et al., “Raster3D Version-2.0—A Program for Photorealistic Molecular Graphics,” Acta Crystallogr. D, 50:869-873 (1994), which is hereby incorporated by reference in its entirety).

FIG. 2 illustrates the stereoview of an electron density map (2Fₒ-F_{c}, 1.2σ, GDP omitted, 2.8-Å resolution) of the GDP-binding pocket. An atomic model of the final structure is embedded in the electron density. Drawing prepared from MOLSCRIPT (Kraulis, “Molscript—A Program to Produce Both Detailed and Schematic Plots of Protein Structures,” J. Appl. Crystallogr., 24:946-950 (1991), which is hereby incorporated by reference in its entirety) and RASTER3D (Merritt et al., “Raster3D Version-2.0—A Program for Photorealistic Molecular Graphics,” Acta Crystallogr. D, 50:869-873 (1994), which is hereby incorporated by reference in its entirety).

FIG. 3 illustrates comparisons between the atomic interactions of GDP with TG (Left) and Ras (Right). Hydrogen bonds and ion pair interactions are shown in dashed lines. The GDP molecule is shown in ball-and-stick. TG and Ras residues are shown in thin sticks. Drawing prepared with MOLSCRIPT (Kraulis, “Molscript—A Program to Produce Both Detailed and Schematic Plots of Protein Structures,” J. Appl. Crystallogr., 24:946-950 (1991), which is hereby incorporated by reference in its entirety) and RASTER3D (Merritt et al., “Raster3D Version-2.0—A Program for Photorealistic Molecular Graphics,” Acta Crystallogr. D, 50:869-873 (1994), which is hereby incorporated by reference in its entirety).

FIG. 4 shows sequence alignment of different members of the human transglutaminase family with TG numbering on the bottom. F13A represents Factor XIIIa and B4.2 represents the erythrocyte band 4.2 protein. Conserved residues are in pink; TG catalytic triad residues (Cys-277, His-335, Asp-358) and Tyr-516 are indicated with triangles; residues that interact with bound GDP are indicated with circles. The figure was prepared with ALSCRIPT (Barton, “ALSCRIPT: A Tool to Format Multiple Sequence Alignments,” Protein Eng., 6:37-40 (1993), which is hereby incorporated by reference in its entirety).

FIG. 5 shows the transamidation active site of TG. A close-up view of the juxtaposition of the catalytic triad consisting of Cys-277-His-335-Asp-358 and Tyr-516 relative to the guanine nucleotide-binding site. Cys-277, His-335, Asp-358, Tyr-516, and GDP are shown in ball-and-stick. Tyr-516 points toward Cys-277, the catalytic nucleophile, in the active site. The drawing was prepared by using MOLSCRIPT (Kraulis, “Molscript—A Program to Produce Both Detailed and Schematic Plots of Protein Structures,” J. Appl. Crystallogr., 24:946-950 (1991), which is hereby incorporated by reference in its entirety) and RASTER3D (Merritt et al., “Raster3D Version-2.0—A Program for Photorealistic Molecular Graphics,” Acta Crystallogr. D, 50:869-873 (1994), which is hereby incorporated by reference in its entirety).

FIG. 6 shows comparison of the calcium-binding sites of TG (green) and Factor XIIIa (red). In Factor XIIIa, the loop involved in calcium binding is oriented toward the Ca²⁺-binding site, whereas in TG-GDP, the same loop is oriented toward GDP. The figure was-prepared with MOLSCRIPT (Kraulis, “Molscript—A Program to Produce Both Detailed and Schematic Plots of Protein Structures,” J. Appl. Crystallogr., 24:946-950 (1991), which is hereby incorporated by reference in its entirety) and RASTER3D (Merritt et al., “Raster3D Version-2.0—A Program for Photorealistic Molecular Graphics,” Acta Crystallogr. D, 50:869-873 (1994), which is hereby incorporated by reference in its entirety).

FIG. 7 sets forth the atomic coordinates that defines the three-dimensional crystal structure of tissue transglutaminase, as shown on http://www.rcsb.org/pdb/cgi/explore.cgi?job=download;pdbld=1KV3;page=;pid=141661046120300;opt=show;format=PDB;pre=1&print=1, which is hereby incorporated by reference in its entirety.

FIG. 8 illustrates that epidermal growth factor (EGF) receptor activation stimulates intracellular signaling in SKBR3 cells. NIH3T3 cells were serum starved for 1 day and SKBR3 cells were serum starved for 3 days and were subsequently treated with serum-free medium (0) or 100 ng/ml EGF (EGF) for 5 minutes and lysed. Western blot analysis was performed on the cell extracts to determine the expression levels of the EGF receptor (EGFR) and actin (actin) and the activities of the EGF receptor (P-EGFR), AKT (P-AKT), and ERK (P-ERK).

FIGS. 9A-B show that EGF potently induces TG expression and activation in SKBR3 cells. Cells were grown for 2 days in complete media and subsequently placed in low serum. The following day, cells were left untreated (0), treated with 5 μM RA (RA), 100 ng/ml EGF (EGF), or co-treated with 5 μM RA and 100 ng/ml EGF (RA/EGF) for 2 days and then lysed. In FIG. 9A, the cell extracts were used to determine TG GTP-binding activities (GTP-TGase) using an affinity-labeling assay with radioactive GTP as outlined in Example 11. Western blot analysis using TG and actin antibodies was performed to assess the expression levels of each of these proteins (TGase and actin). In FIG. 9B, the same cell lysates were assayed for TG transamidation activities as determined by the incorporation of 5-(biotin-amido) pentylamine into proteins as described in Example 12. The experiments were conducted in triplicate, quantitated, and the values from each experiment were averaged together and plotted. In FIG. 9C, SKBR3 cells were seeded at 1×10⁵ cells/well and grown in low serum medium +/−5 μM RA and +/−100 ng/ml EGF for the times indicated and then counted. The results from three independent growth assays were averaged together and plotted.

FIGS. 10A-C show that LY294002 treatment inhibits the ability of EGF and RA to induce TG expression and GTP-binding activity in SKBR3 cells. Cells were grown to near confluence and were subsequently placed in low serum medium +/−6 μM LY294002. In FIG. 10A, SKBR3 cells were treated with 100 ng/ml EGF for 5 minutes, lysed, and then assayed for activated AKT (P-AK7) and actin (actin) by Western blot analysis. In FIG. 10B, cells pretreated with or without LY2940025 (LY) were treated +/−5 μM RA or +/−100 ng/ml EGF for 2 additional days before being lysed. The cell extracts were used to determine the expression levels of TG (TGase) and actin (actin) via Western blot analysis, as outlined in Example 10, and TG GTP-binding activity (GTP-TGase) by photoaffinity labeling, as outlined in Example 11. In FIG. 10C, SKBR3 cells transiently transfected with vector only (vector) or a HA-tagged myristolated form of the catalytic subunit of PI3 kinase (HA-M-p110) were grown in low serum medium +/−100 ng/ml EGF for 1.5 days and then lysed. Western blot analysis was performed on the cell lysates to assess the expression levels of the HA-tagged PI3 kinase construct (M-p110), TG (TGase), and actin (actin).

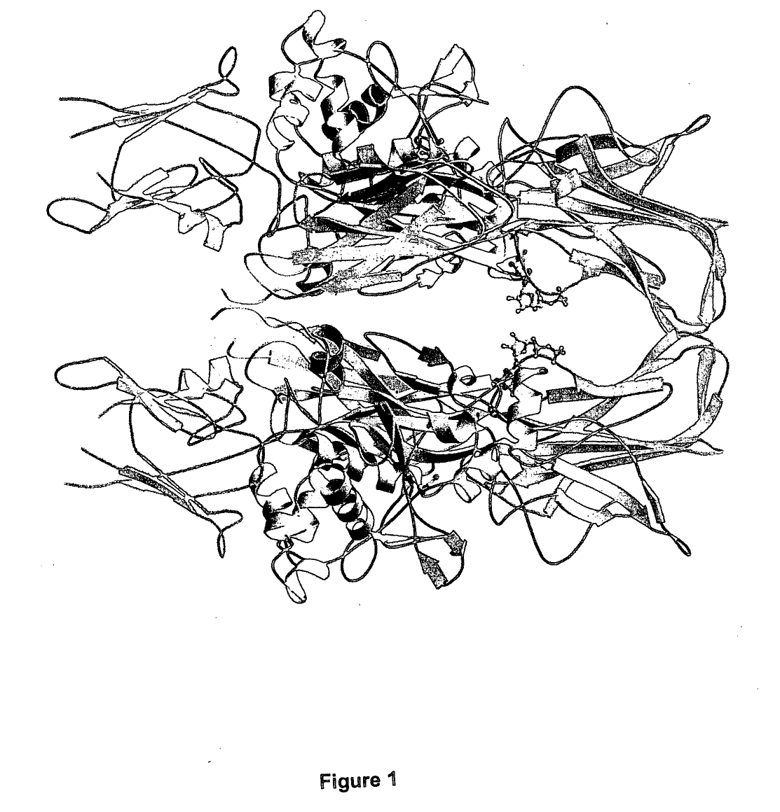

FIGS. 1A-D illustrate that the protective effect of EGF from doxorubicin-induced apoptosis is blocked by MDC in SKBR3 cells. In FIG. 11A, nearly confluent cultures of SKBR3 and MDAMB231 cells were placed in low serum medium. The following day, the cells were exposed to low serum medium +/−0.25 μM doxorubicin (Dox) for the times indicated and subsequently lysed. The presence of the activated form of caspase 3 (cleaved caspase 3) and actin (actin) in the cell extracts was assessed by Western blot analysis. In FIG. 11B, SKBR3 and MDAMB231 cells were maintained in low serum medium (0) or treated with 10 μM MDC (MDC) for 1.5 days. During this incubation, the SKBR3 cell cultures were also stimulated with +/−100 ng/ml EGF or +/−5 μM RA. All cell cultures were then treated with +/−0.25 μM doxorubicin (Dox) for about 1 day and the cells were scored for programmed cell death as described in Example 13. The assay was performed three times and the average percentage of cell death was plotted (FIG. 11C).

FIG. 12 shows that exogenous TG expression in SKBR3 cells is sufficient to inhibit doxorubicin-induced apoptosis. SKBR3 cells transiently transfected with vector only (vector), Myc-tagged wild-type TG (WT TG), or a Myc-tagged dominant-negative form of TG (TG s171e) were grown in low serum medium +/−100 ng/ml EGF. The following day, the cells were treated with +/−0.25 μM doxorubicin (Dox) for another day and the cells expressing the various constructs were scored for programmed cell death as described in Example 13. The assay was performed in triplicate and the average percentage of cell death was plotted. The inset of FIG. 12 depicts the expression levels of the transiently transfected TG proteins (WT TG and TG s171e) and actin (actin) prior to exposure to doxorubicin.

### DETAILED DESCRIPTION OF THE INVENTION

The present invention relates to a method of facilitating death of cancer cells in a subject. The method involves inhibiting tissue transglutaminase in the subject under conditions effective to facilitate death of cancer cells. In one embodiment of the present invention, the tissue transglutaminase is human tissue transglutaminase.

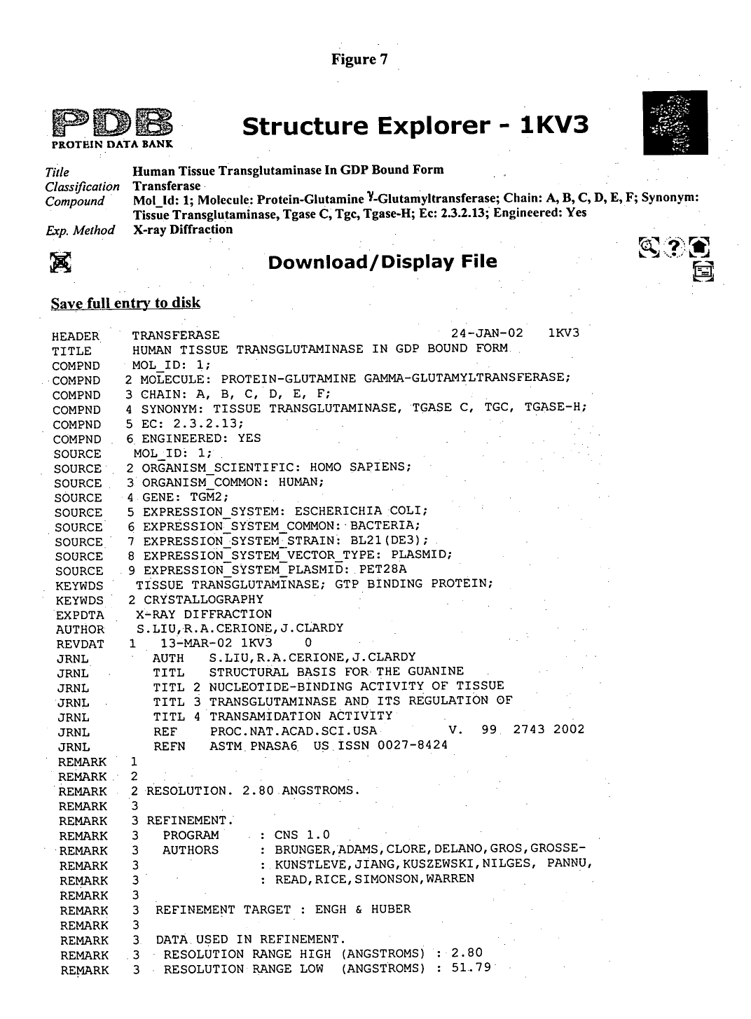

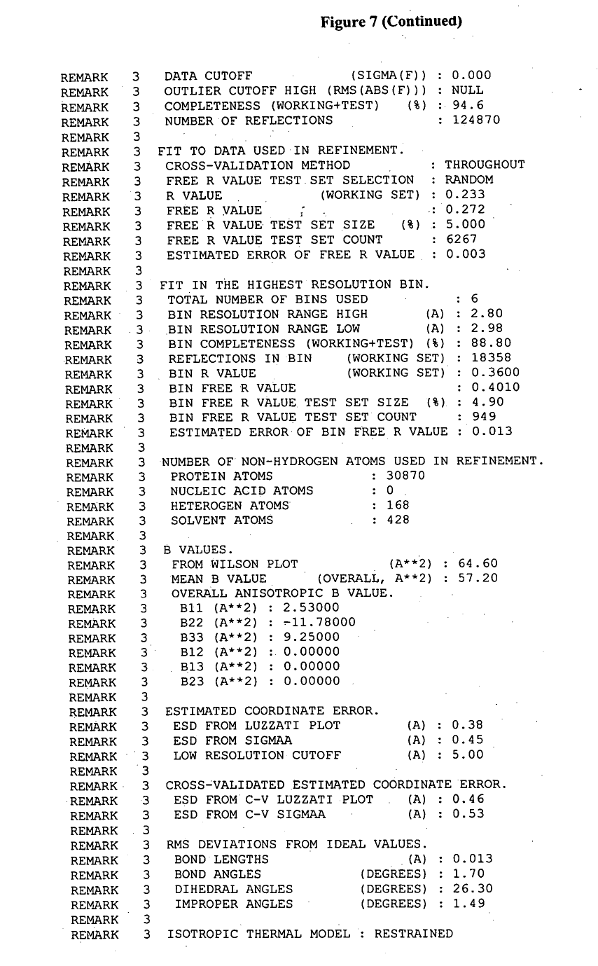

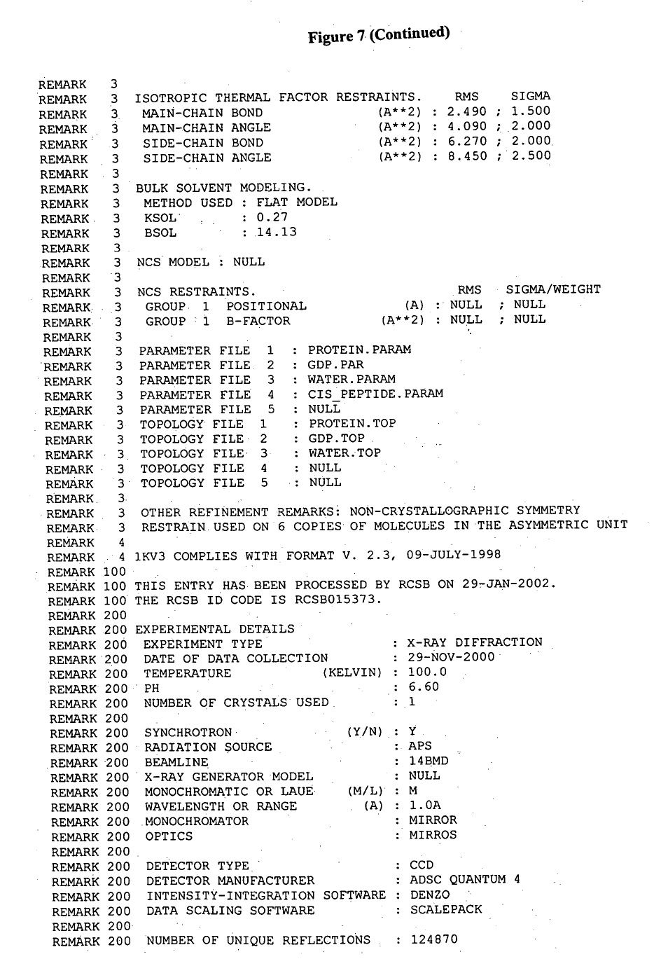

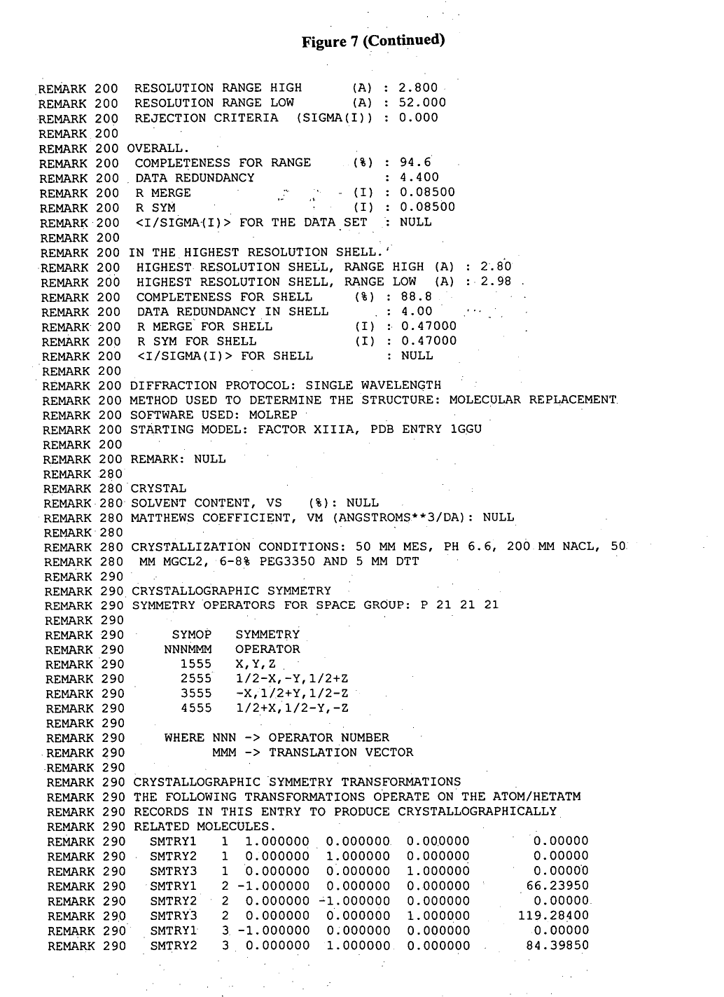

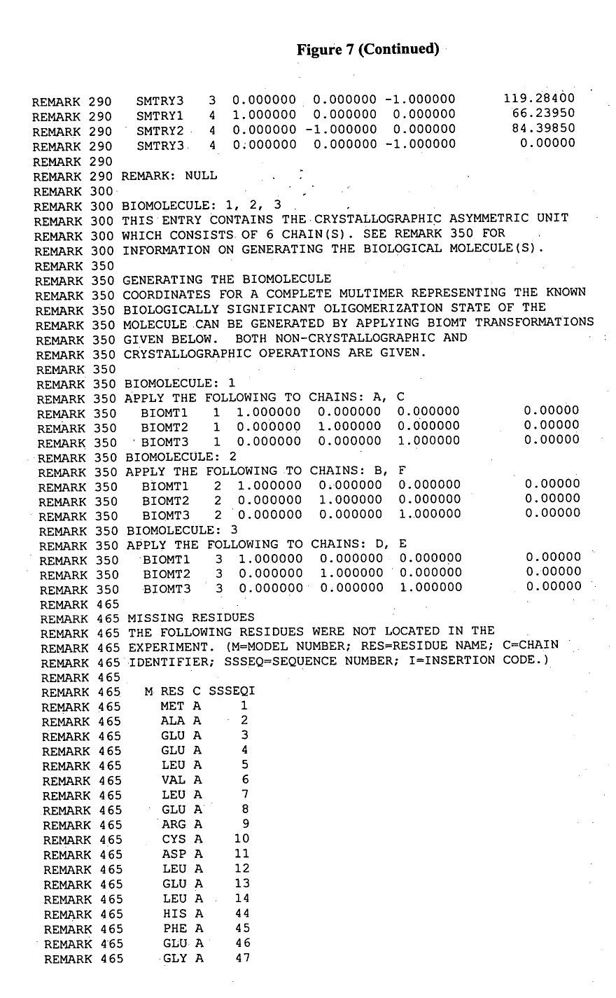

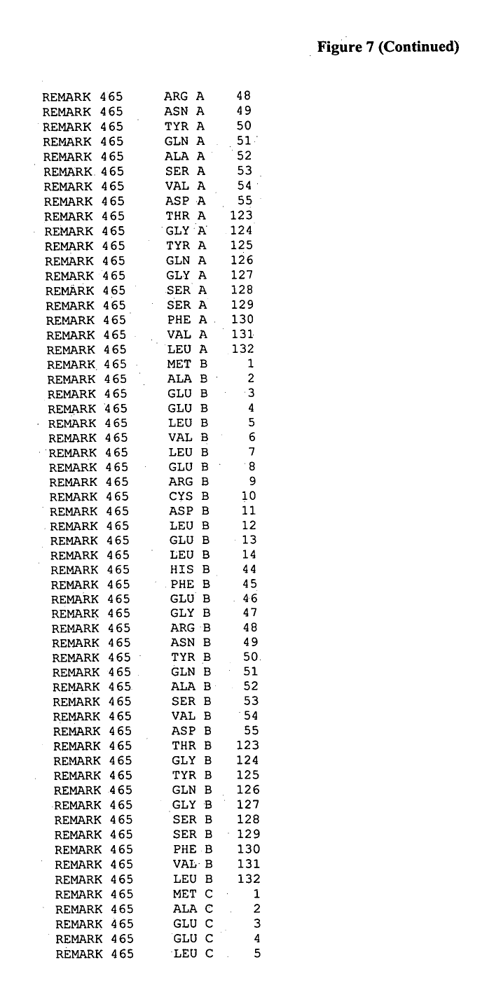

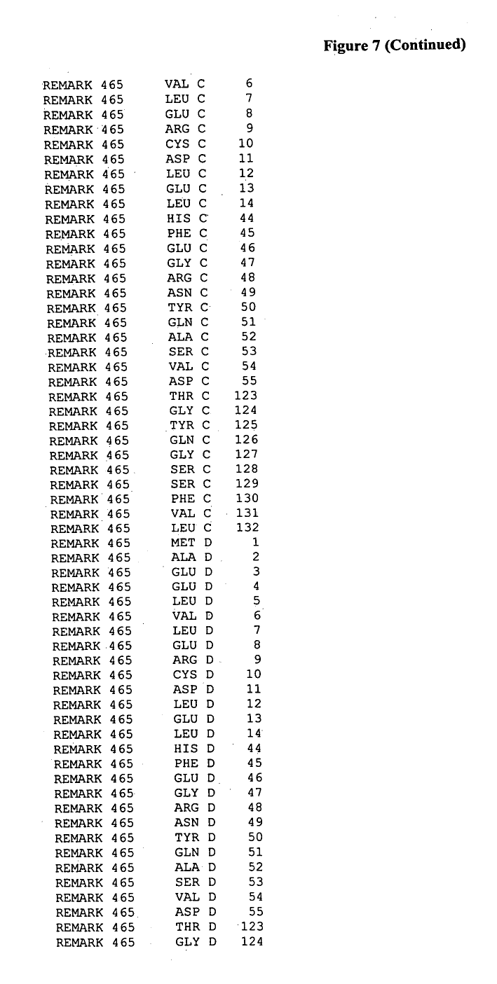

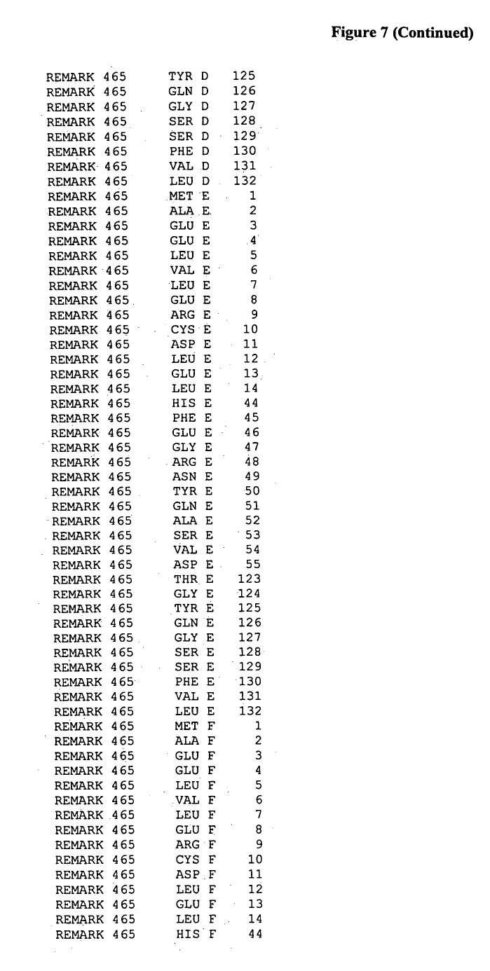

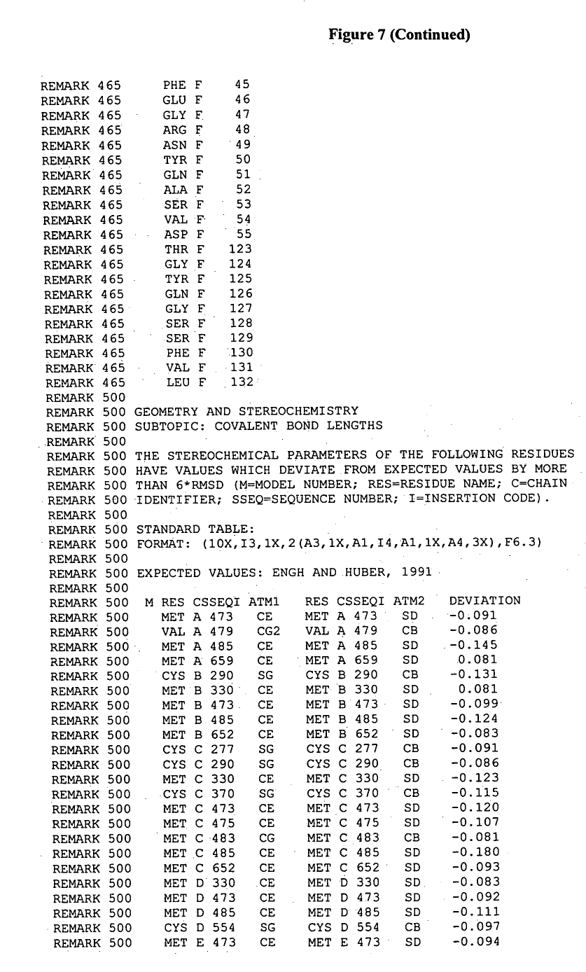

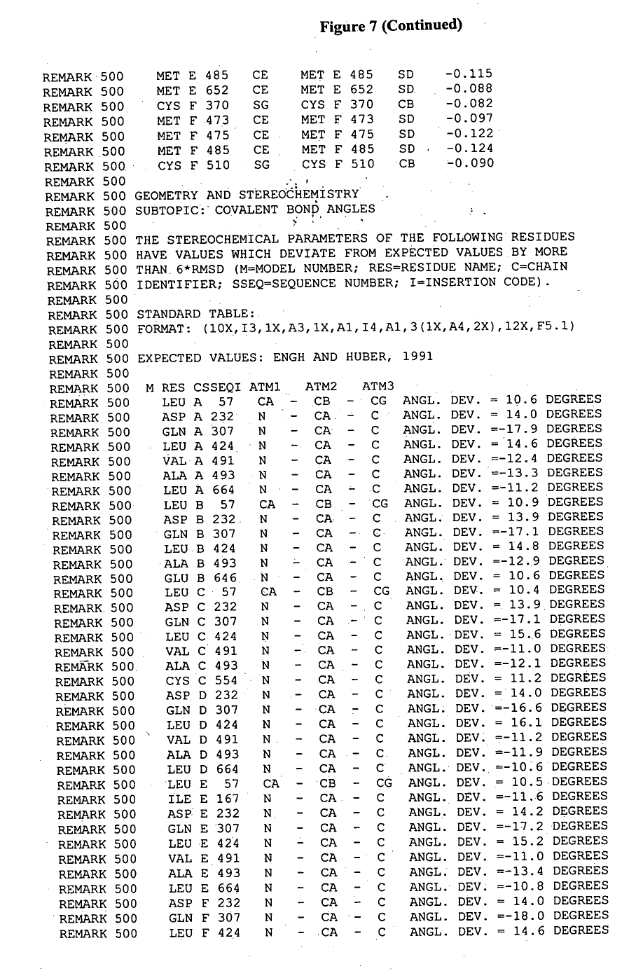

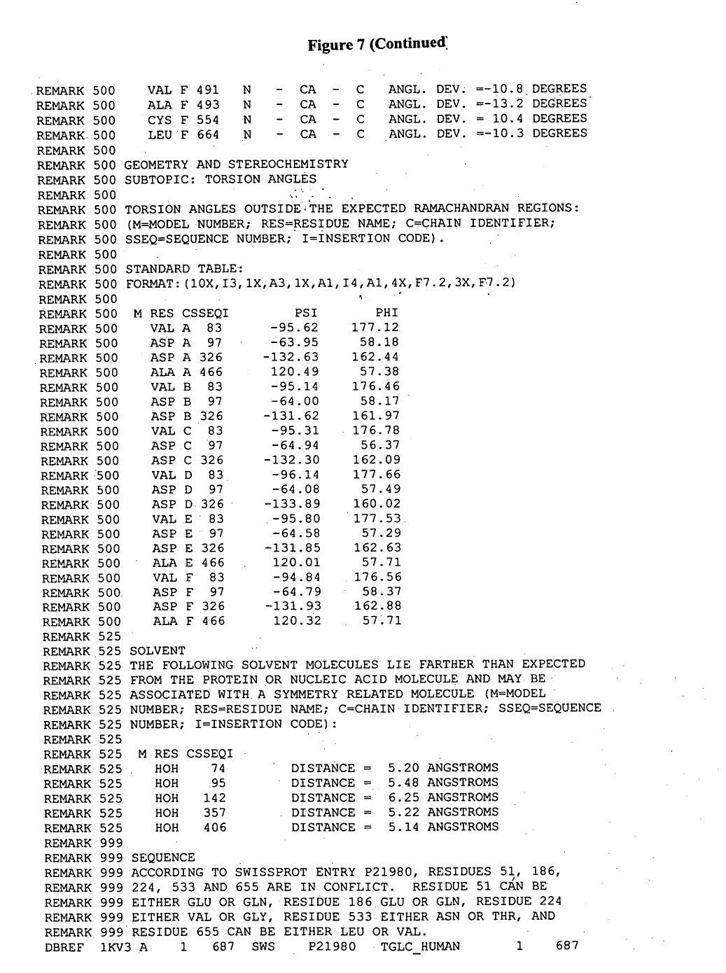

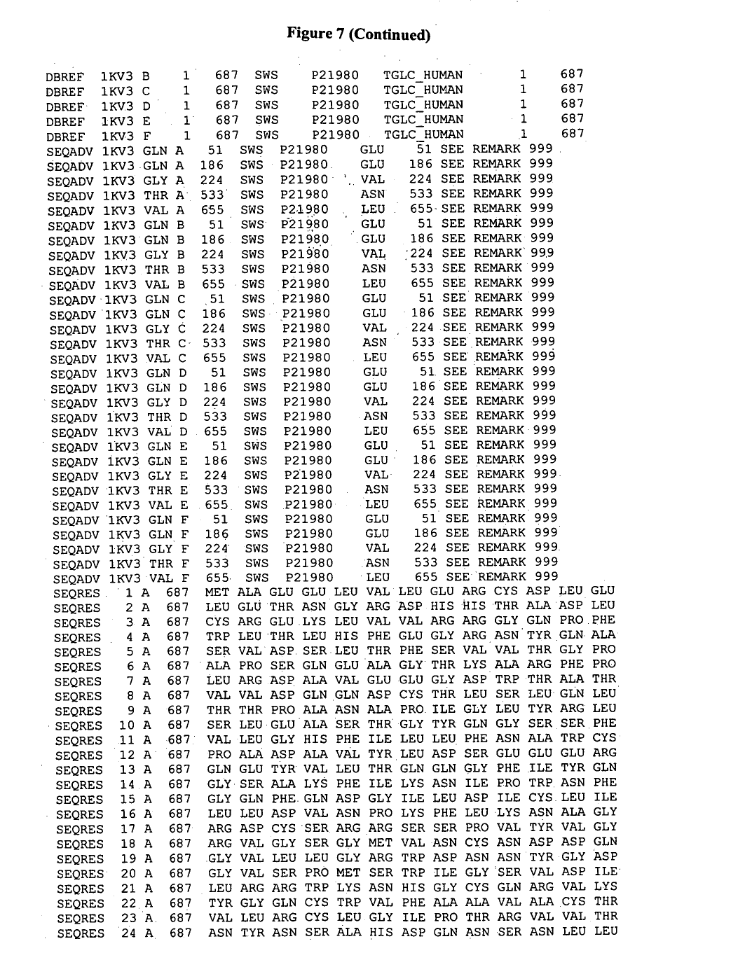

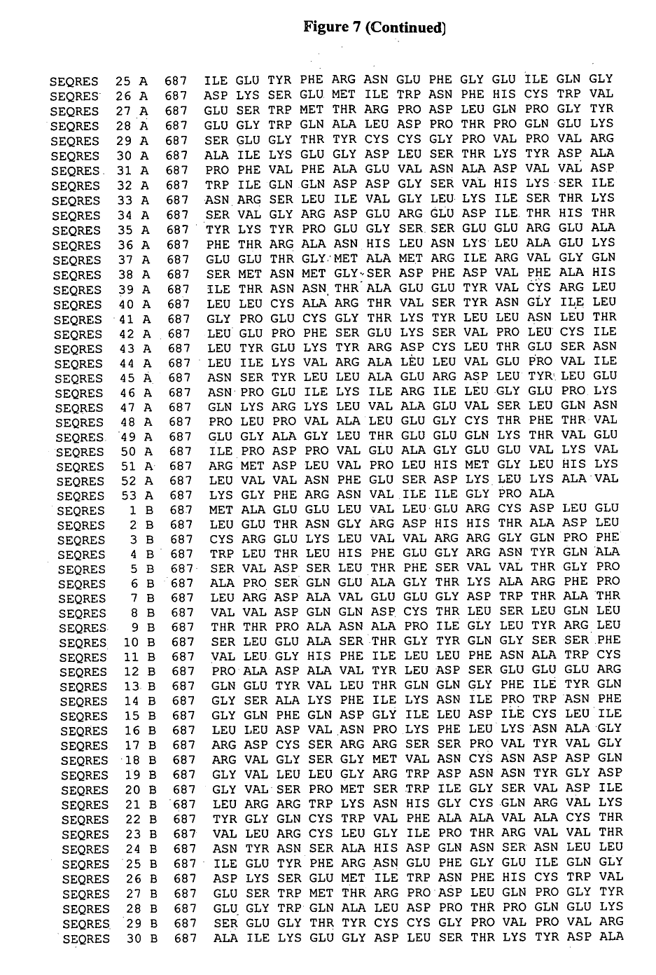

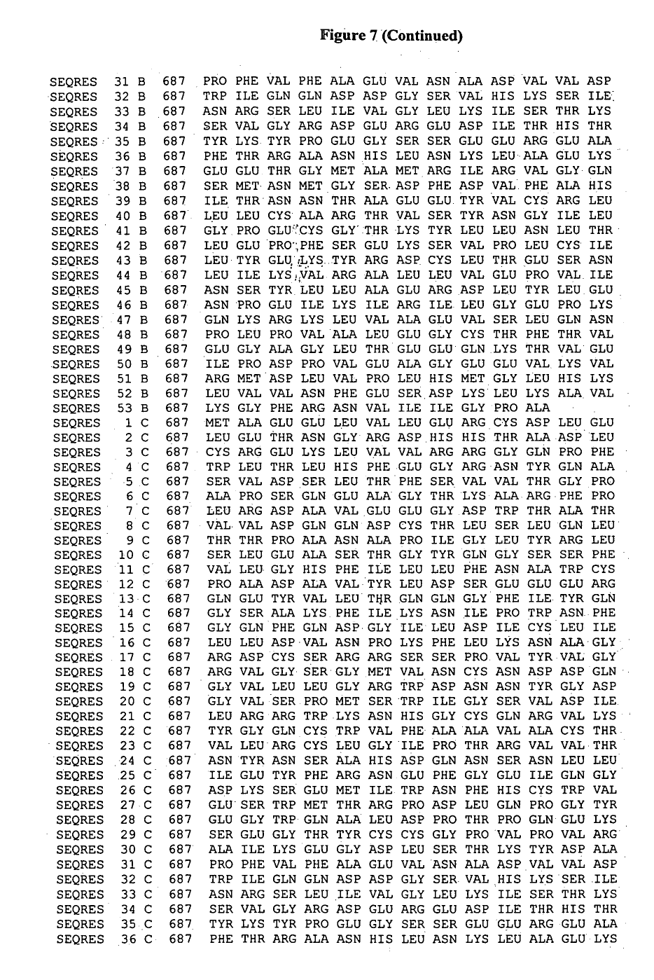

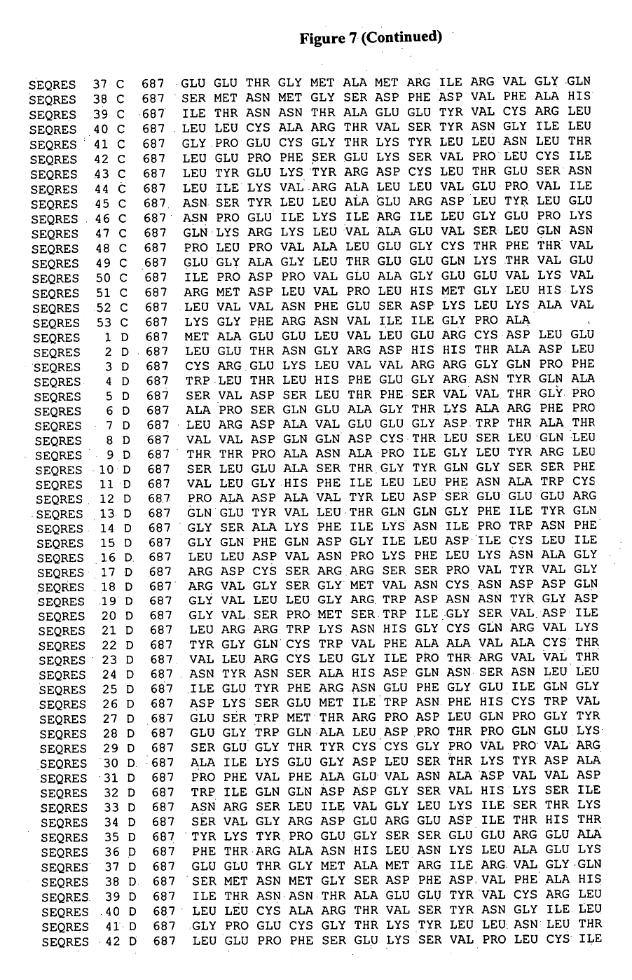

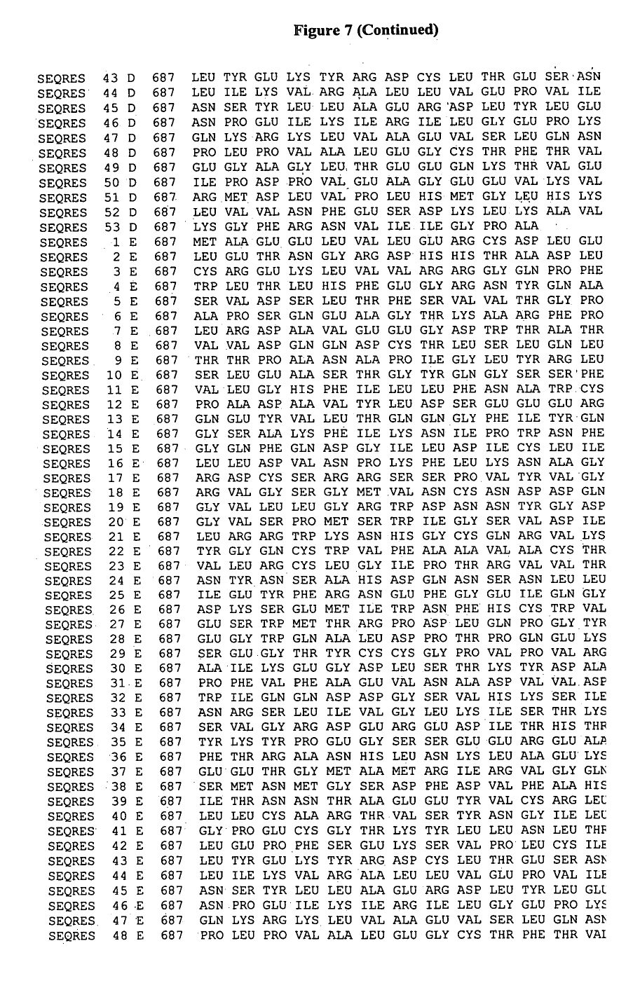

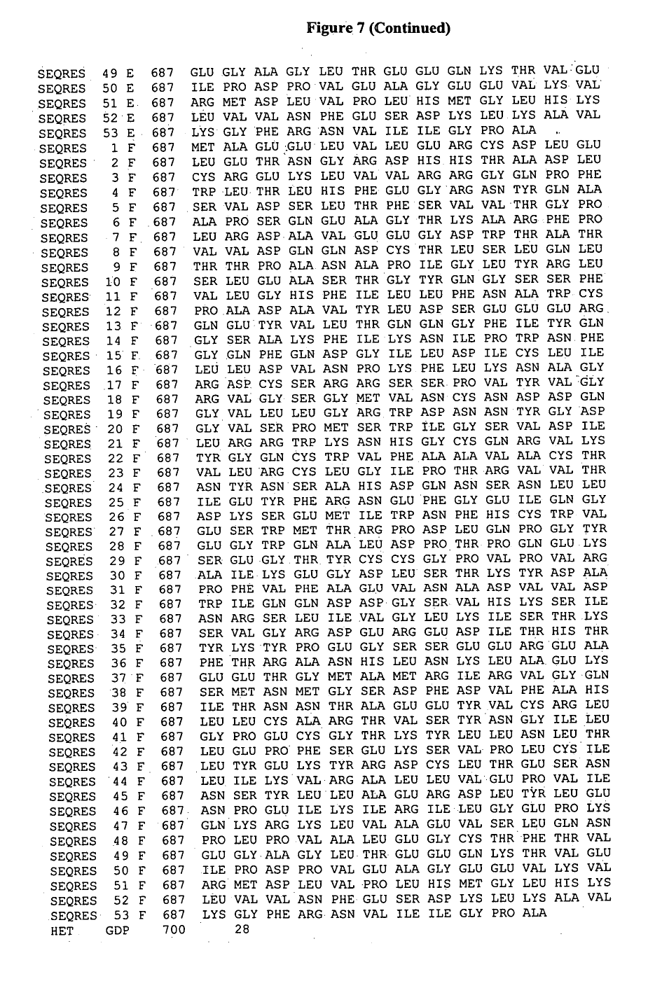

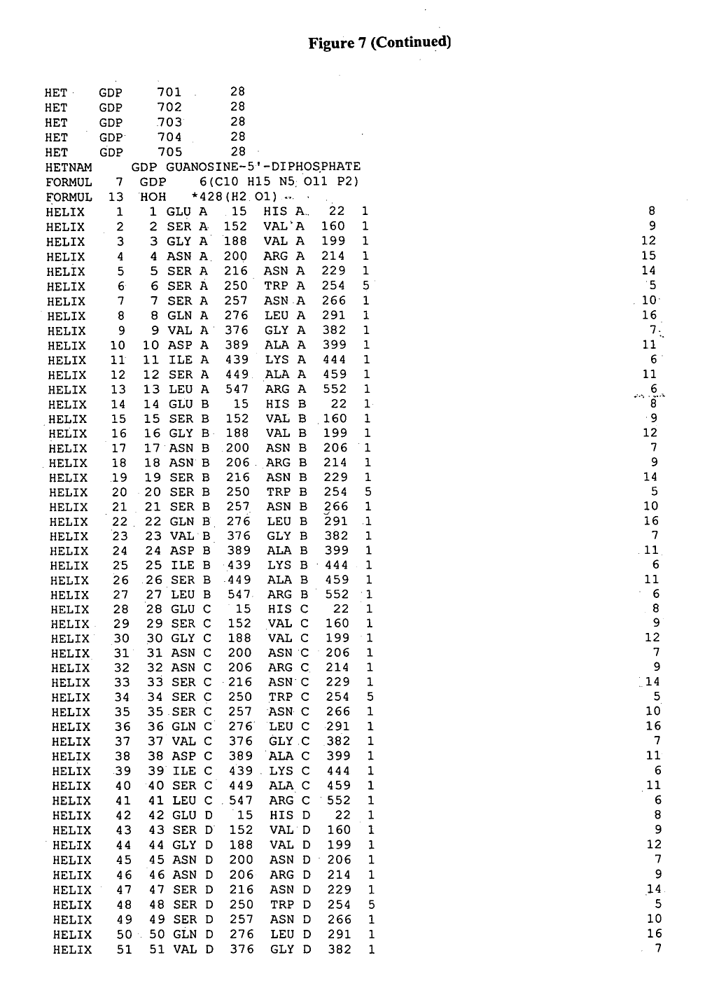

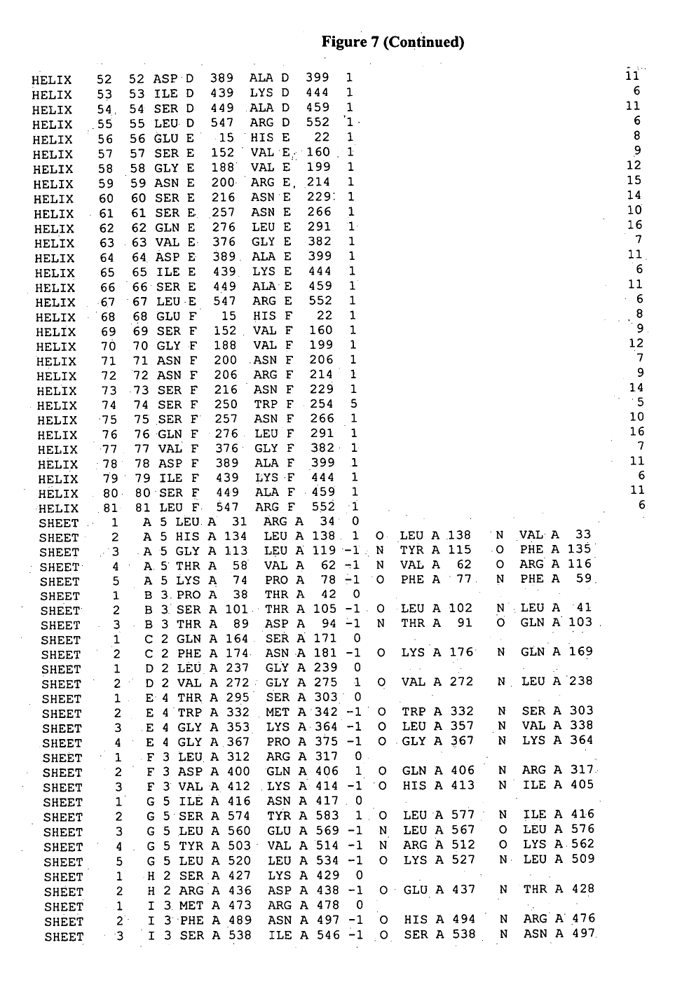

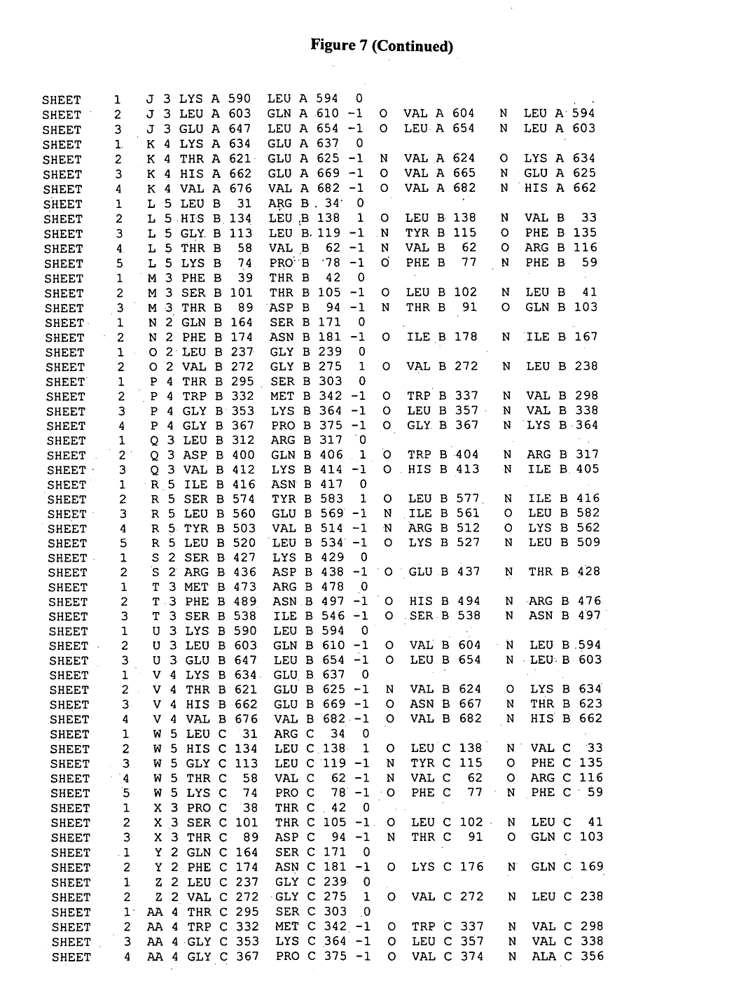

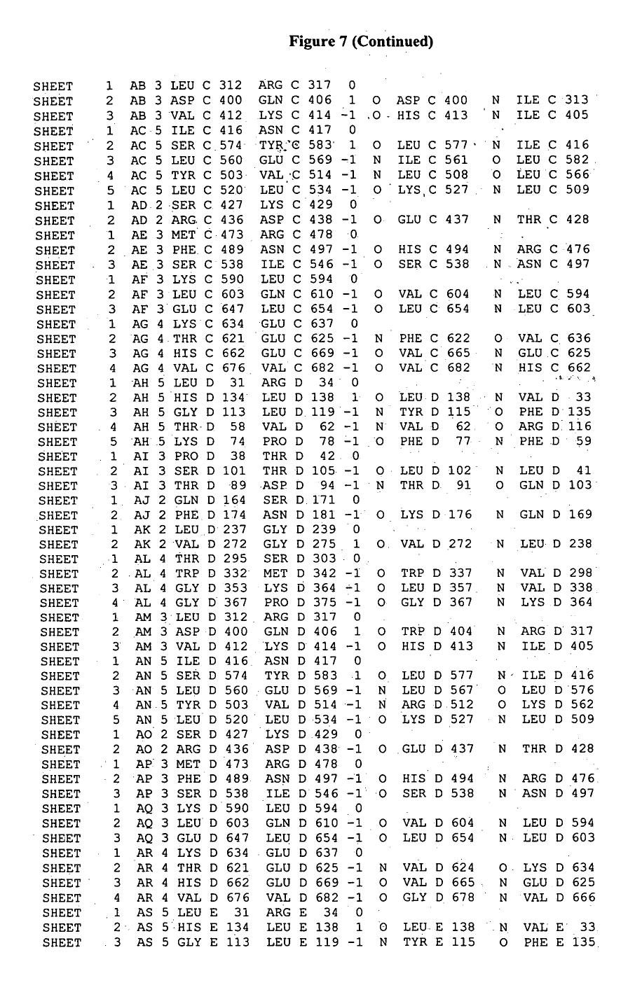

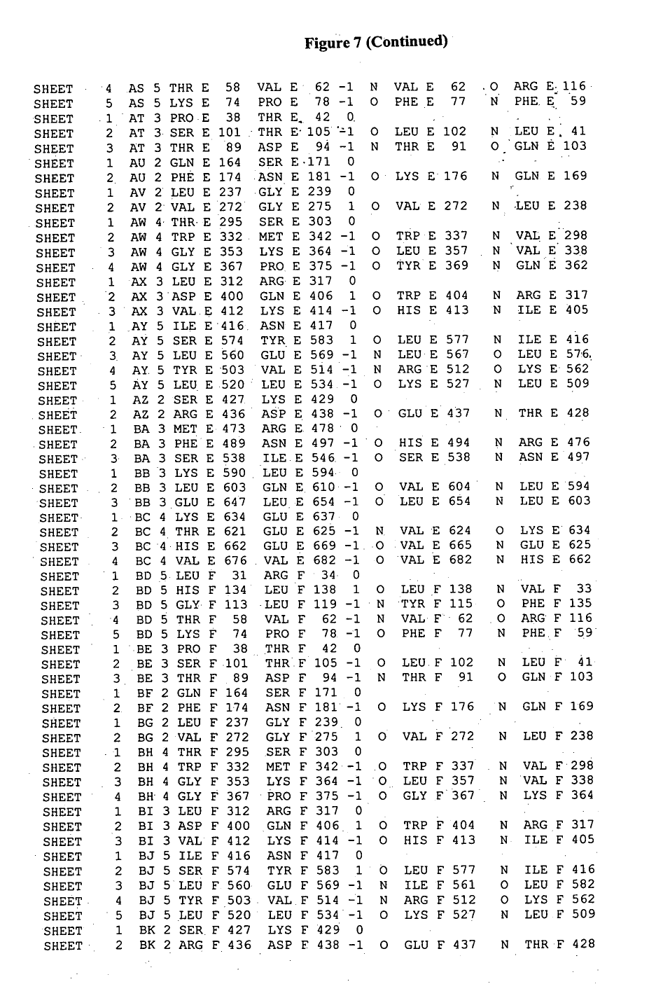

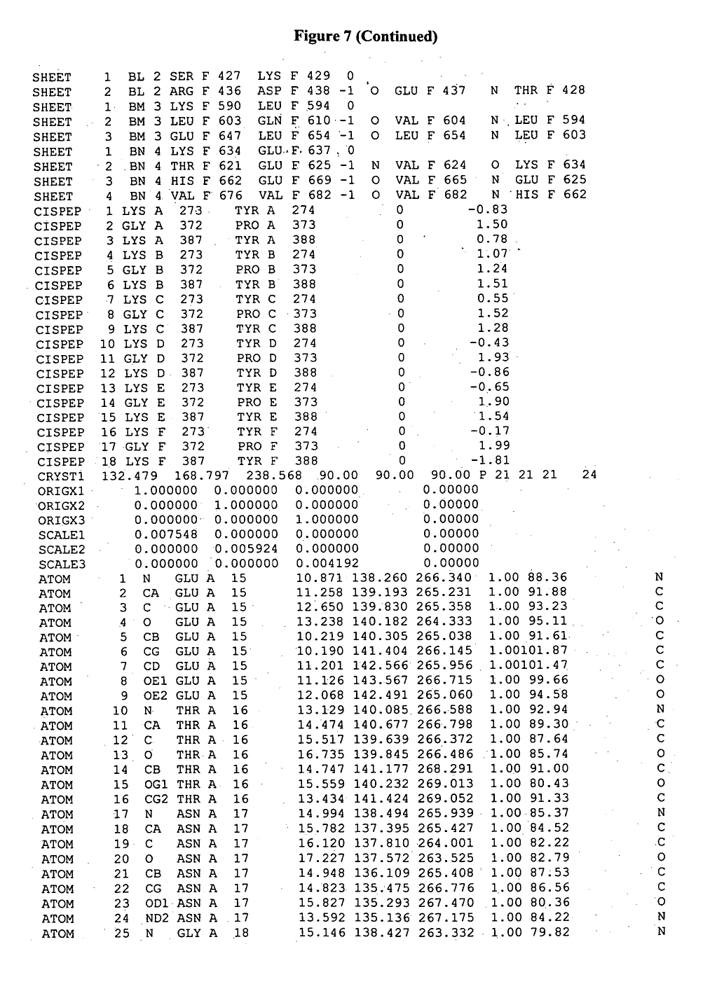

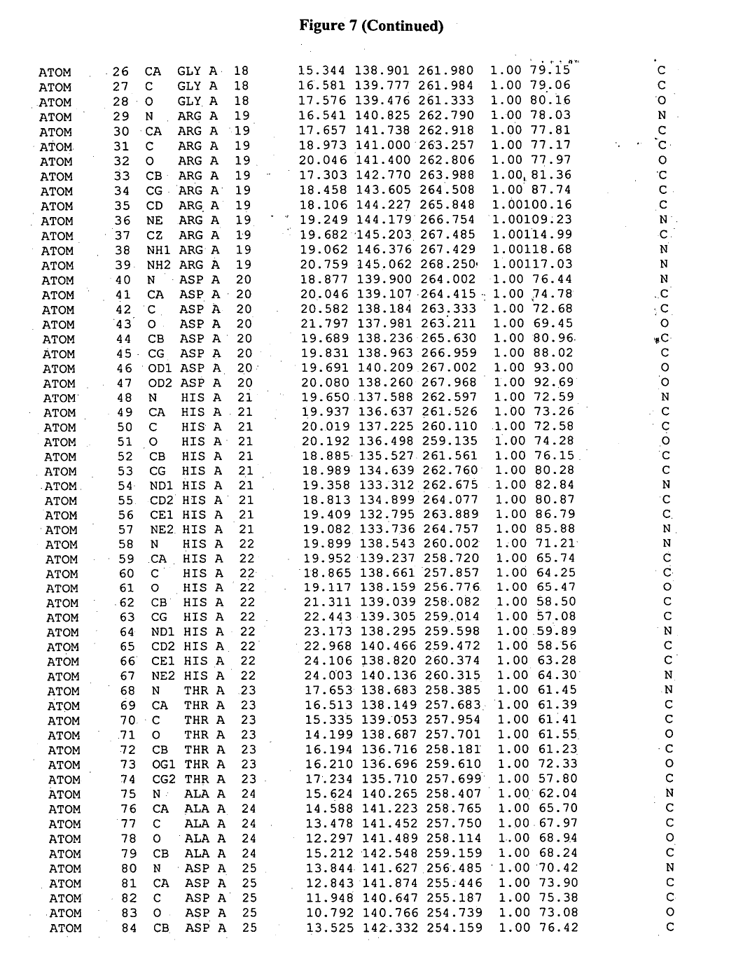

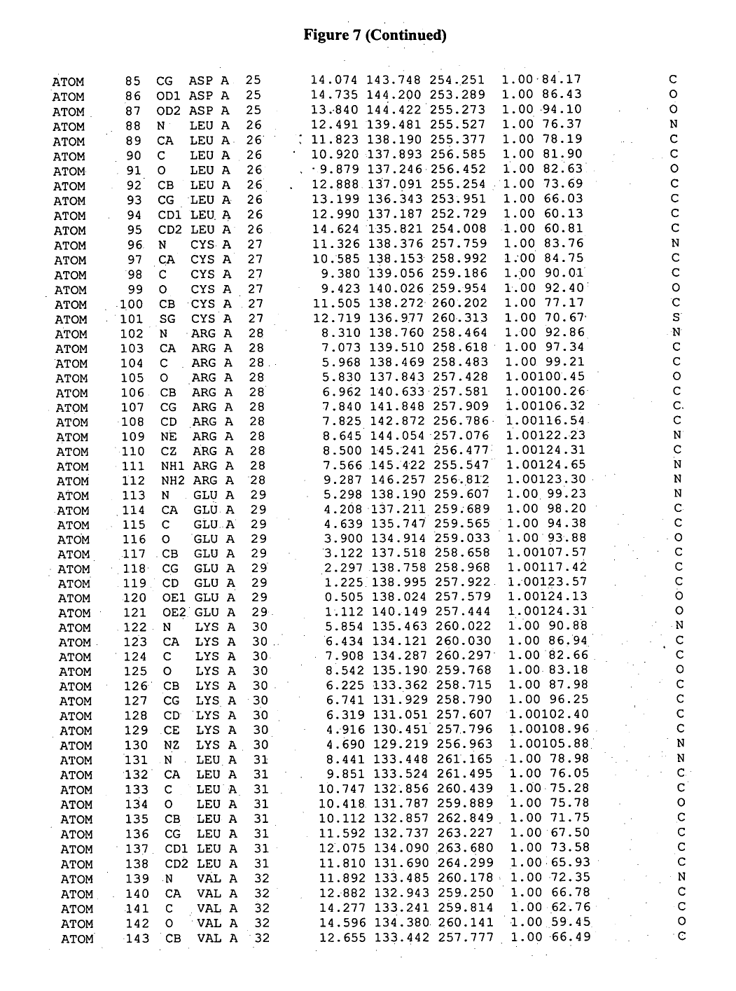

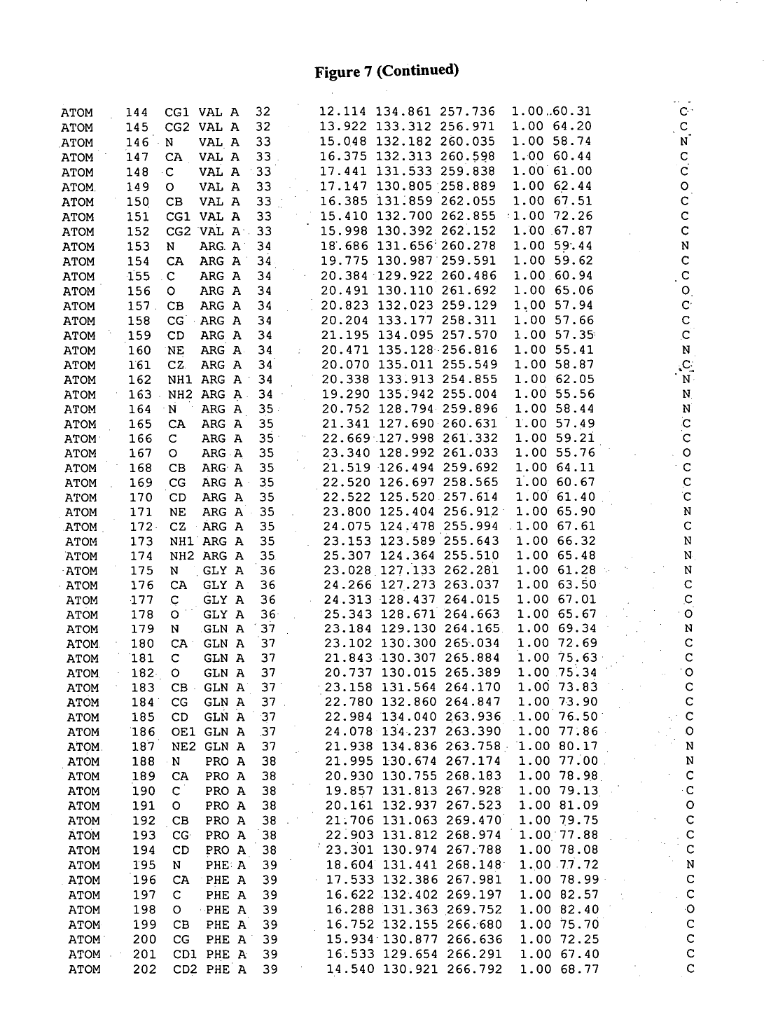

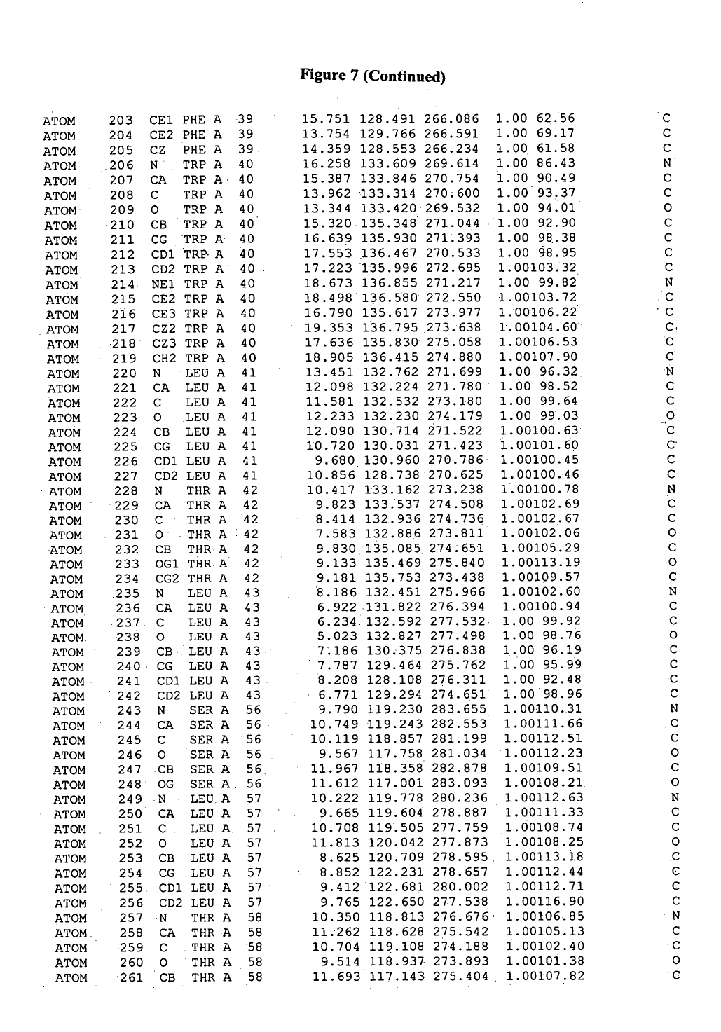

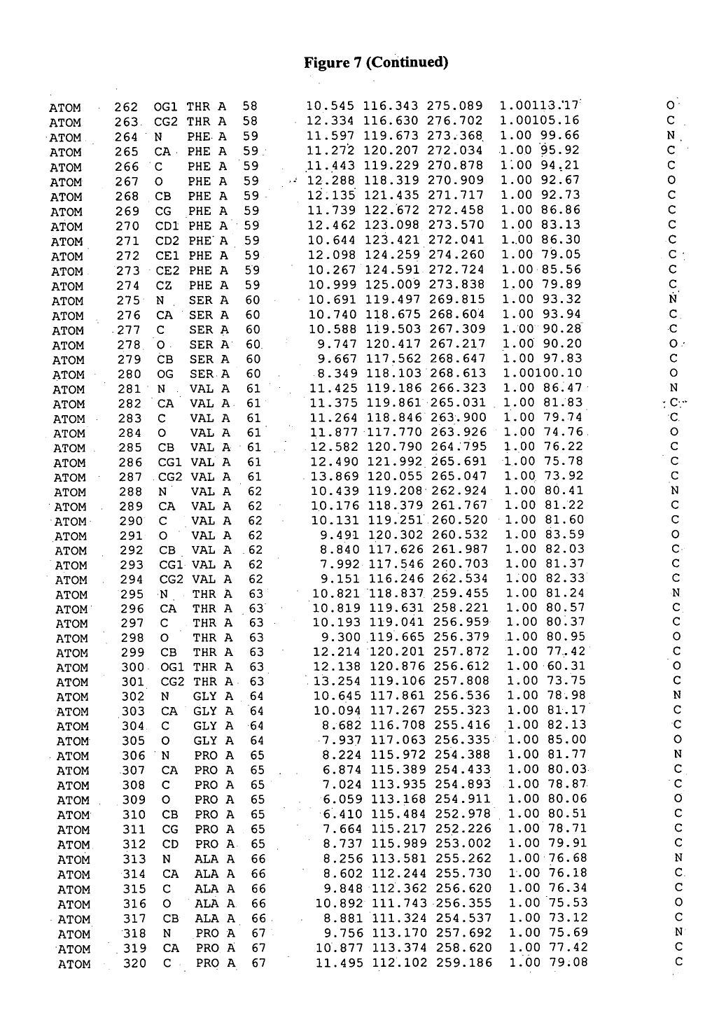

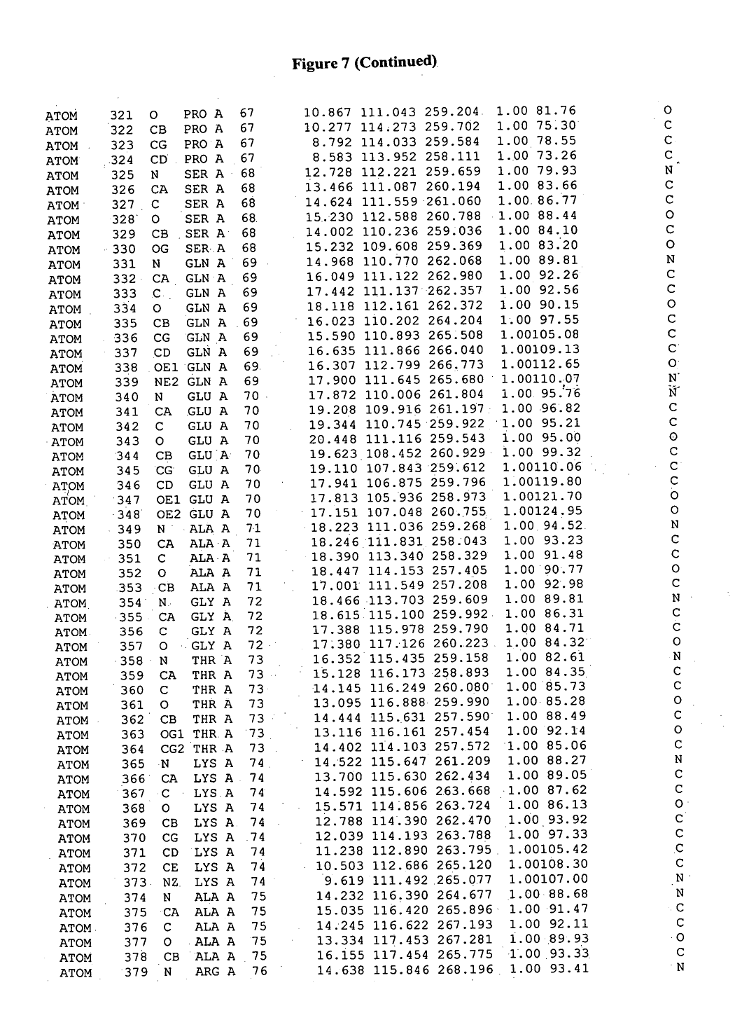

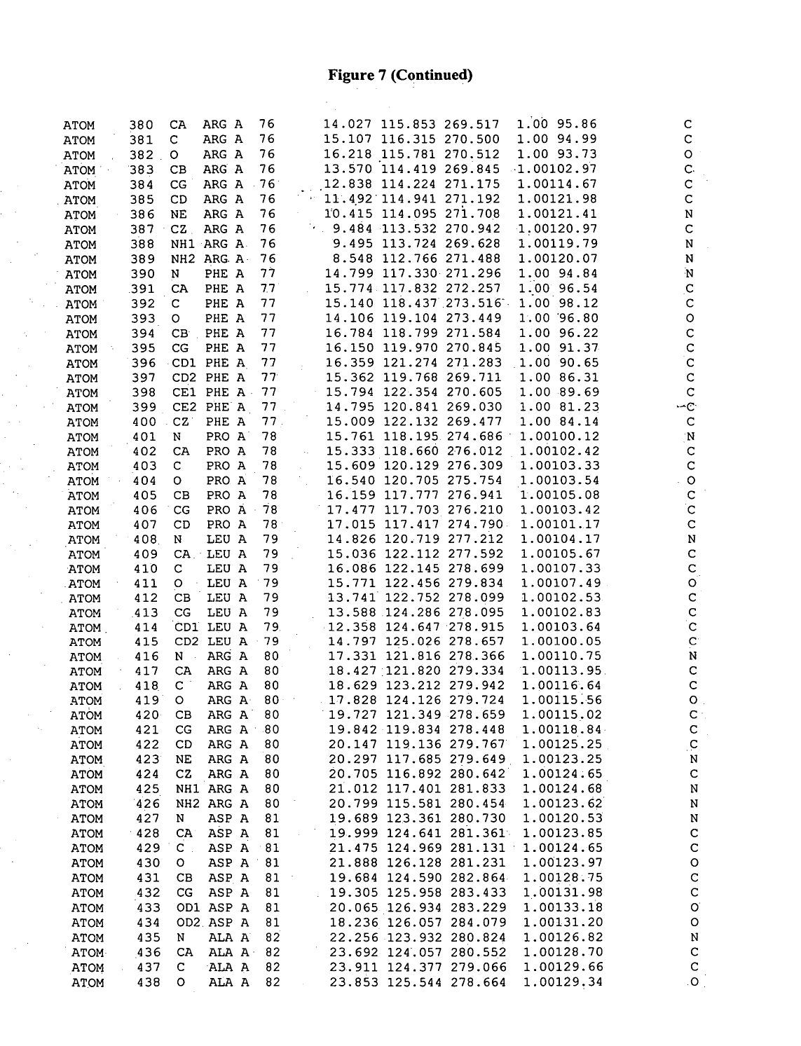

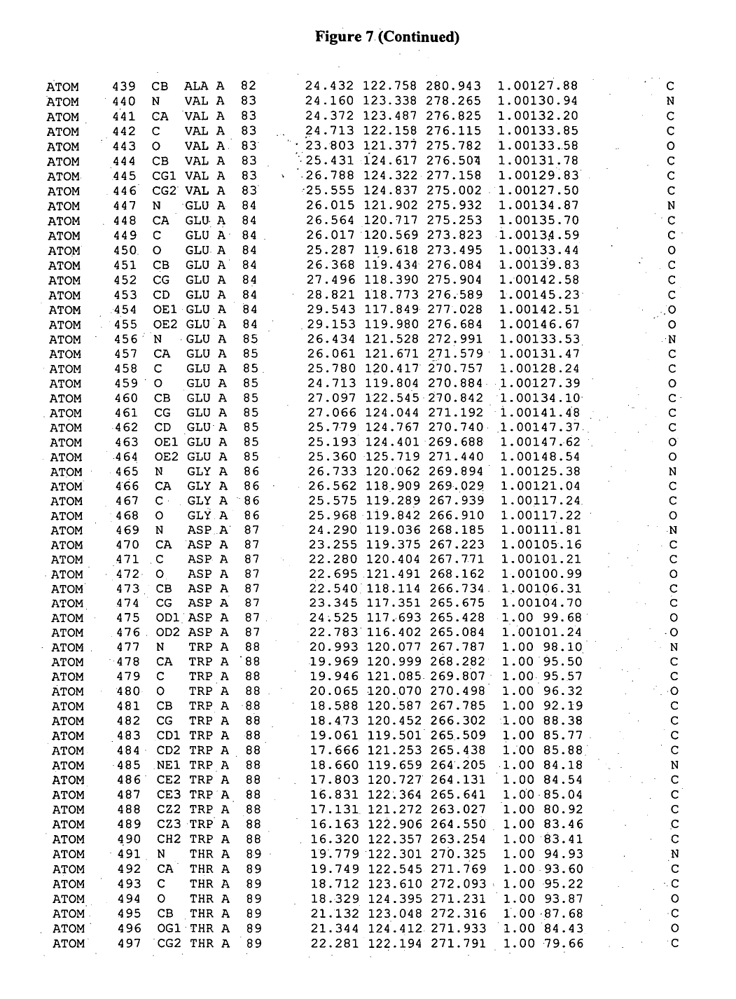

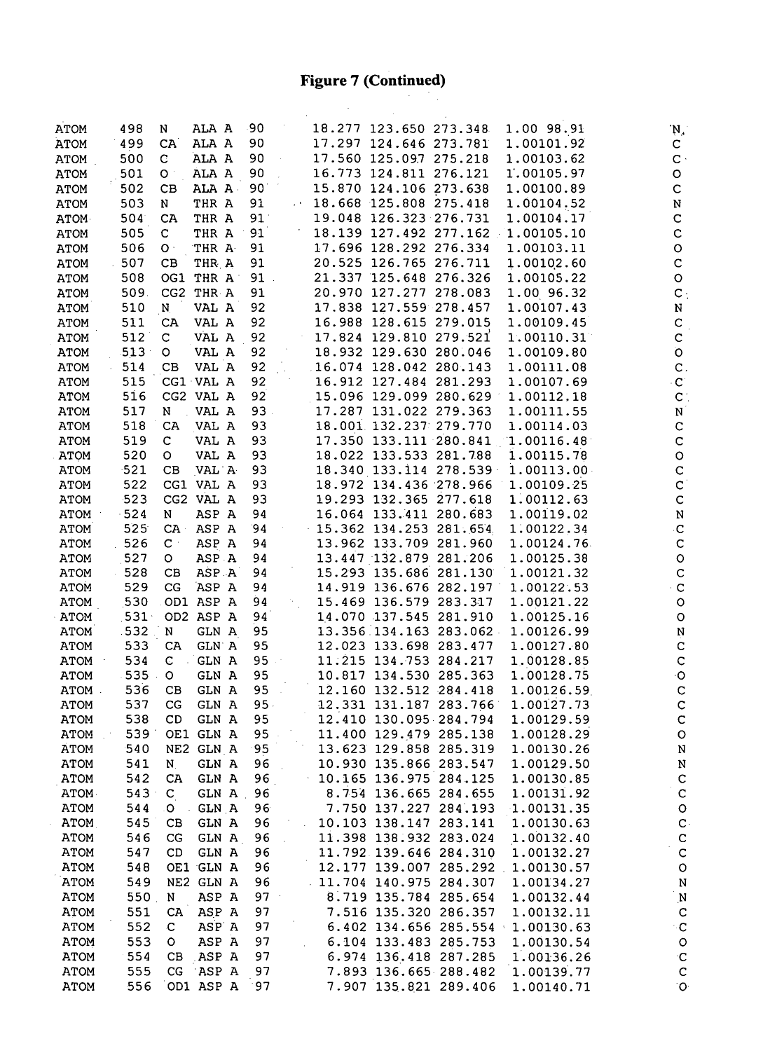

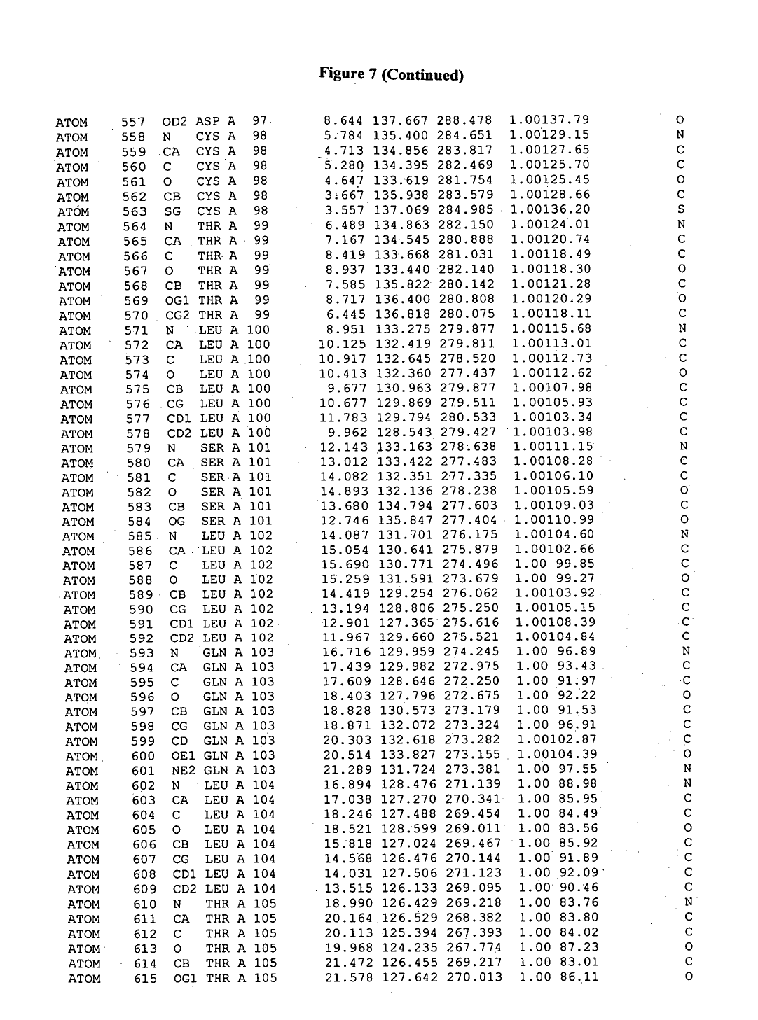

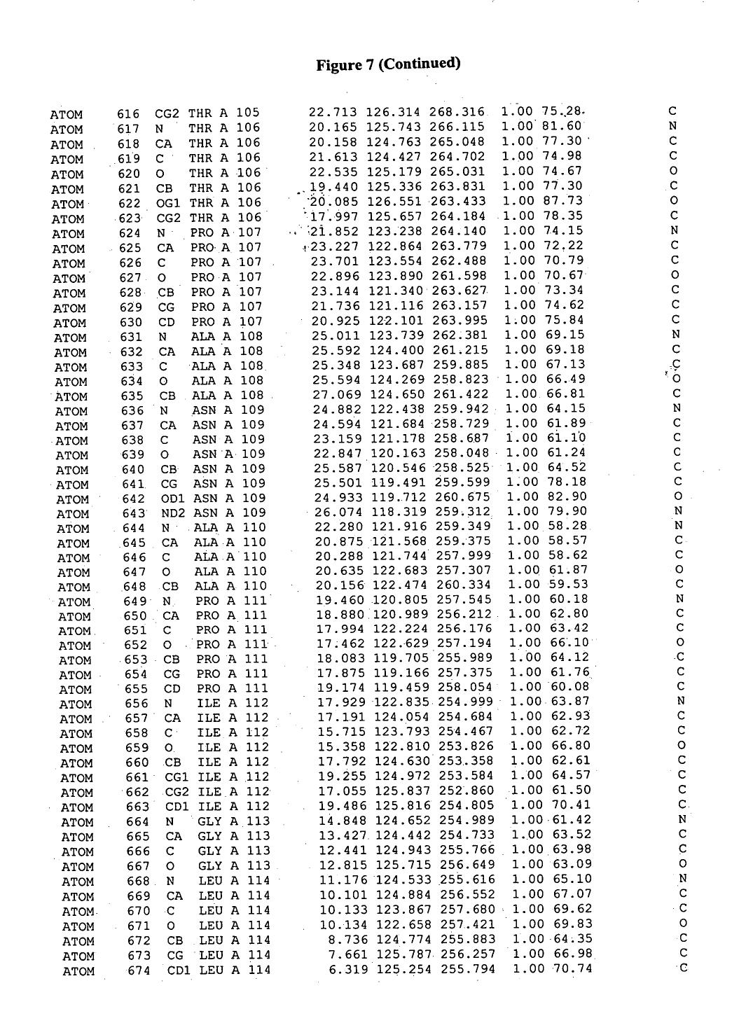

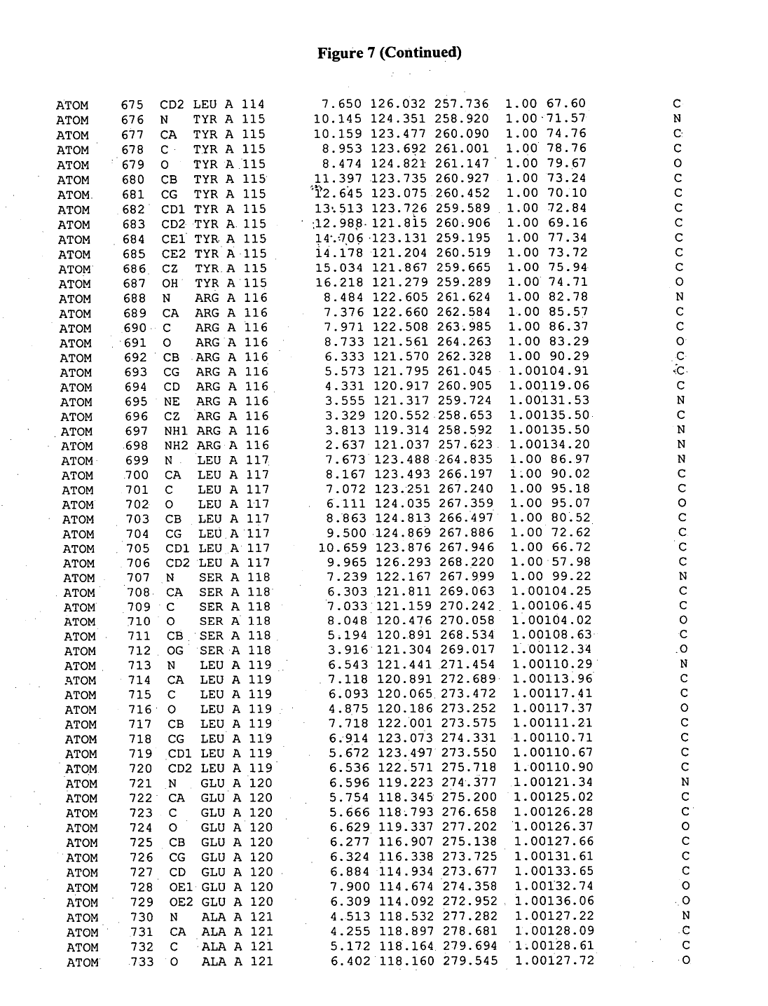

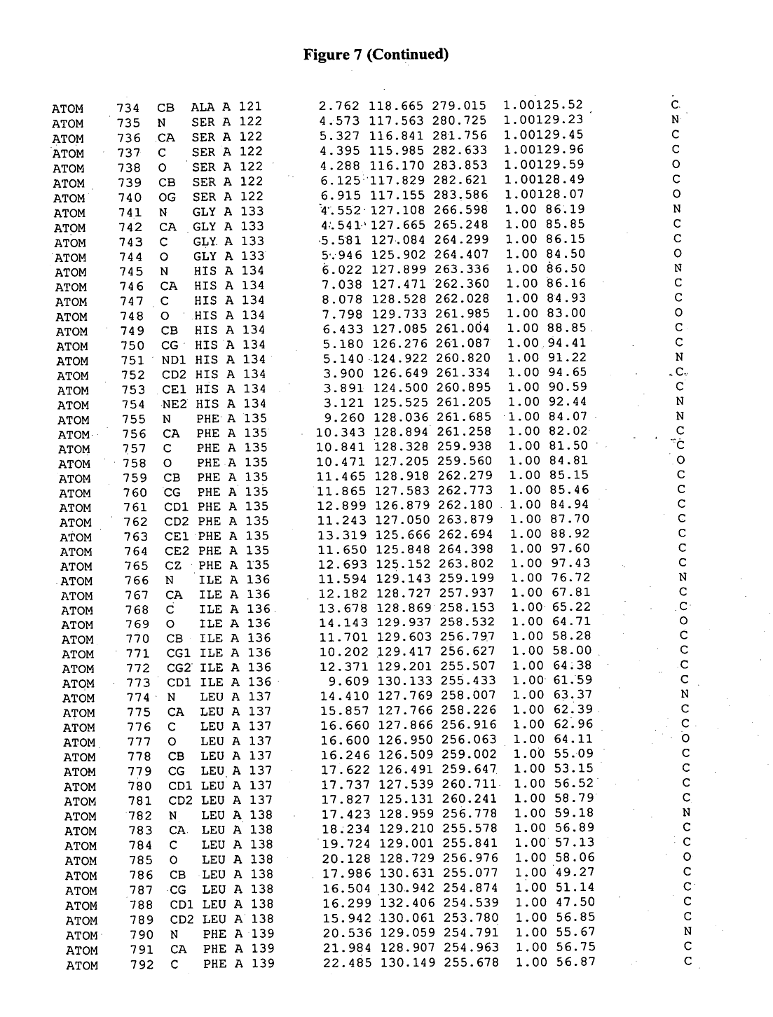

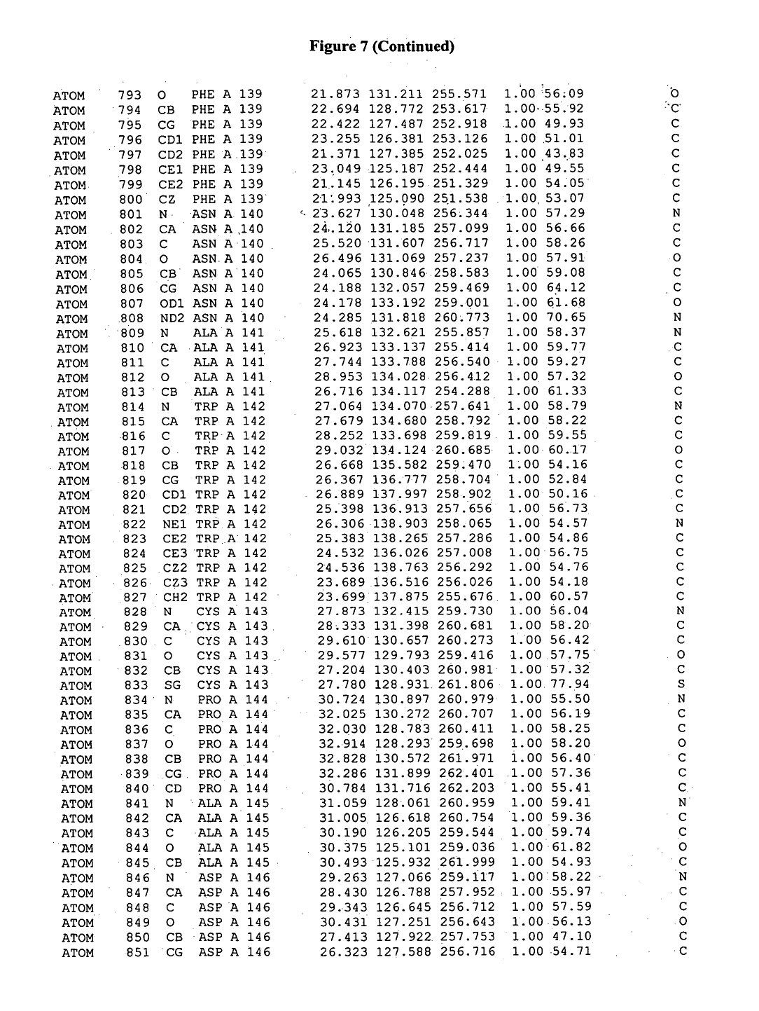

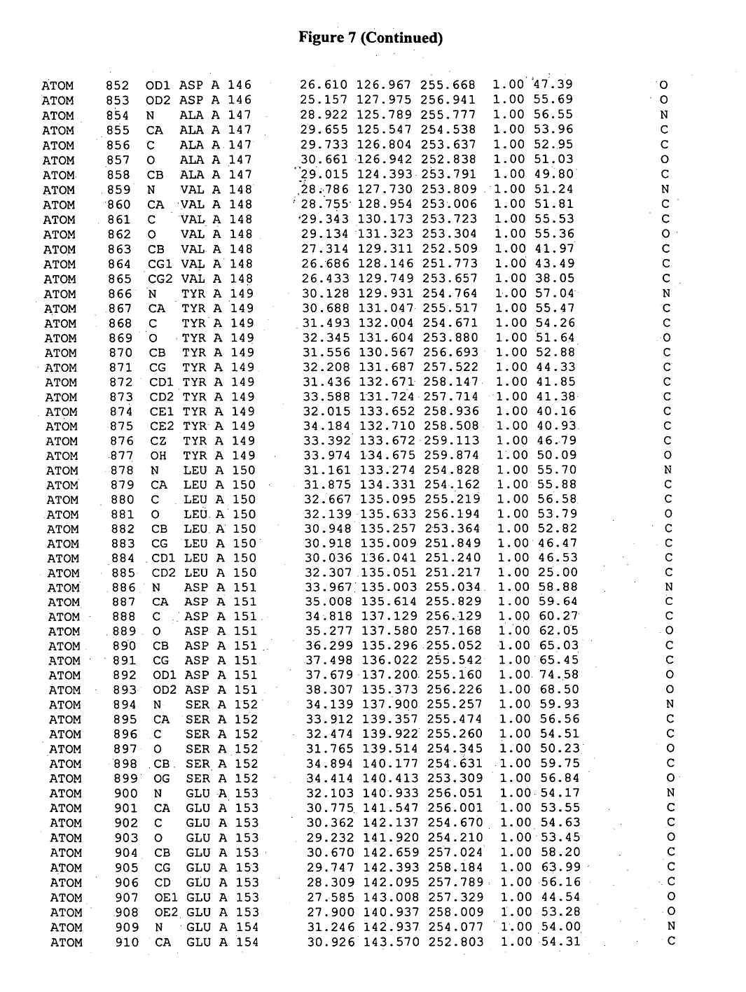

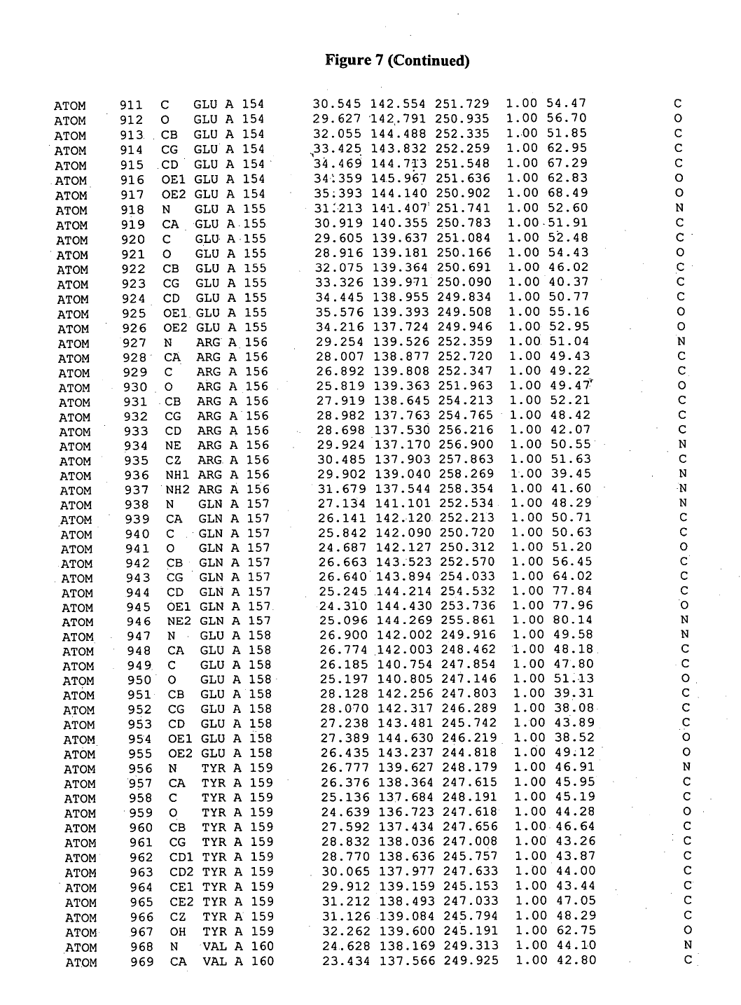

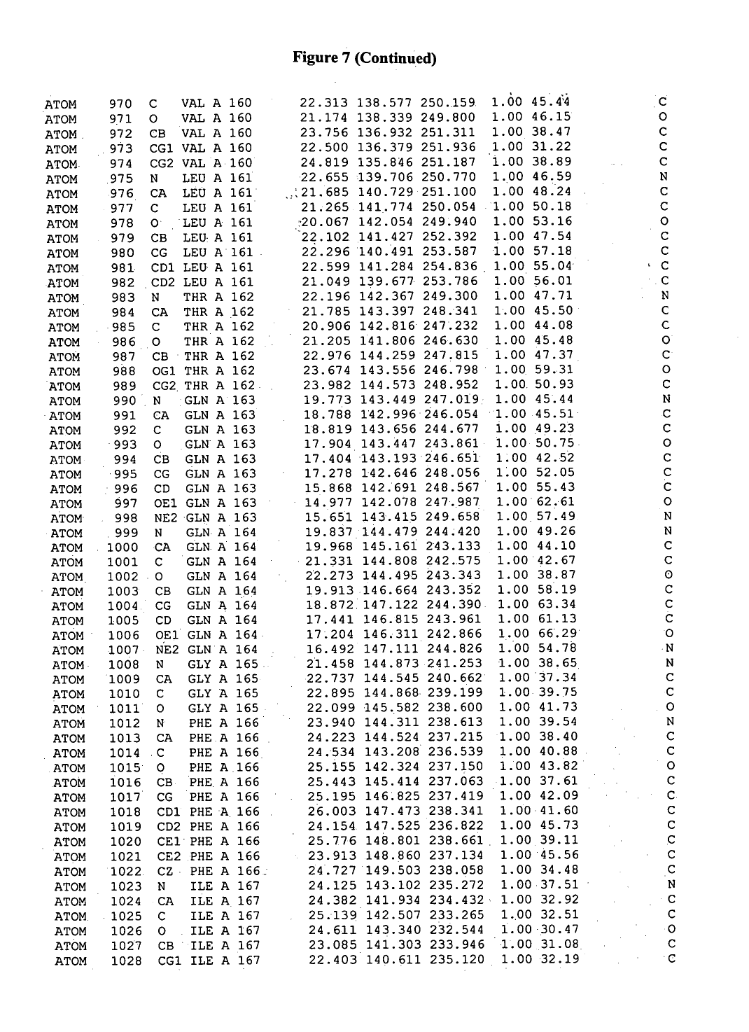

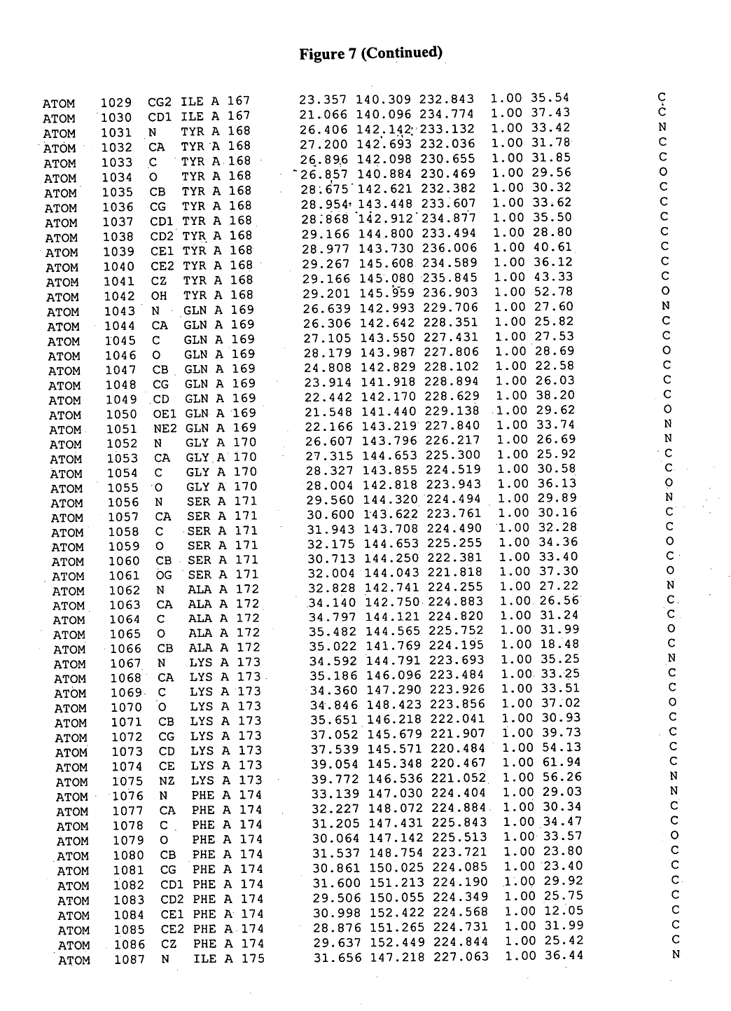

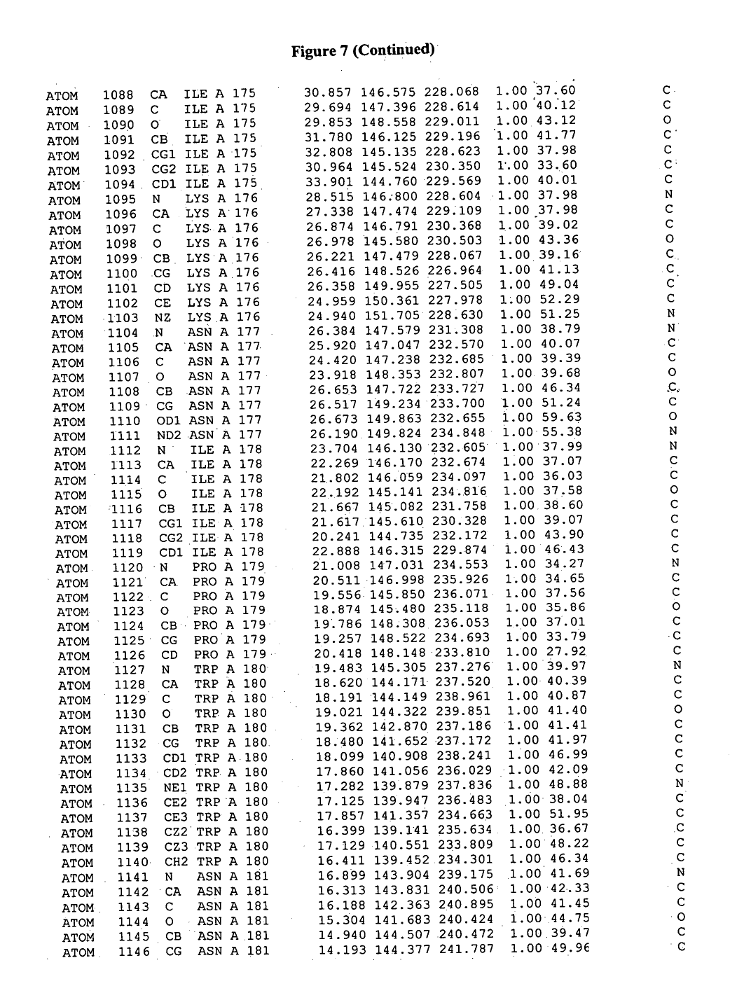

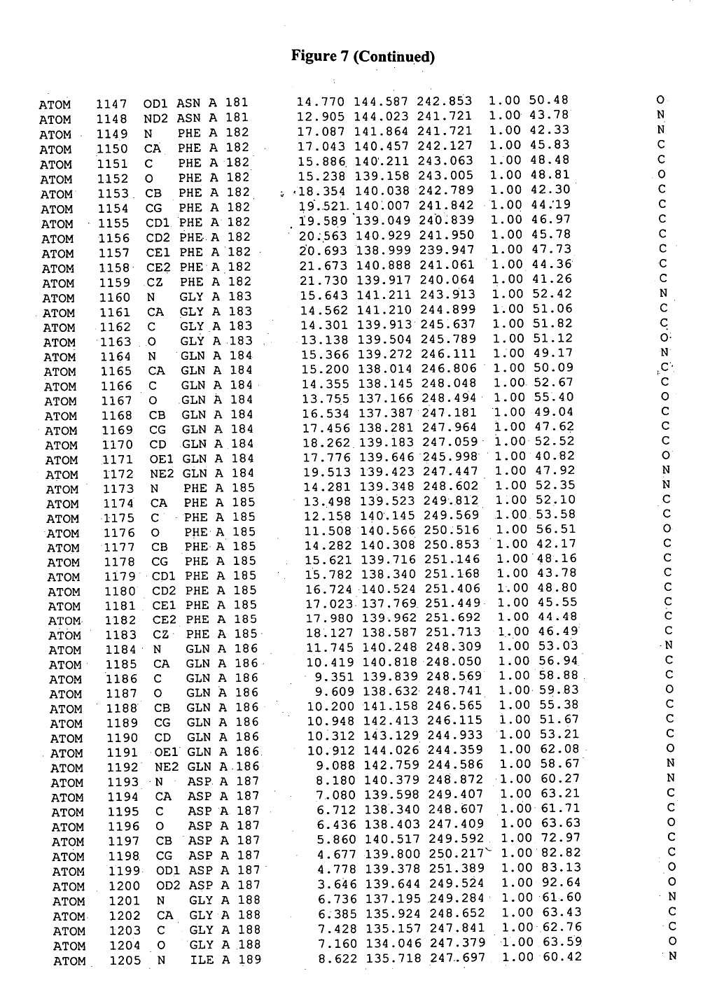

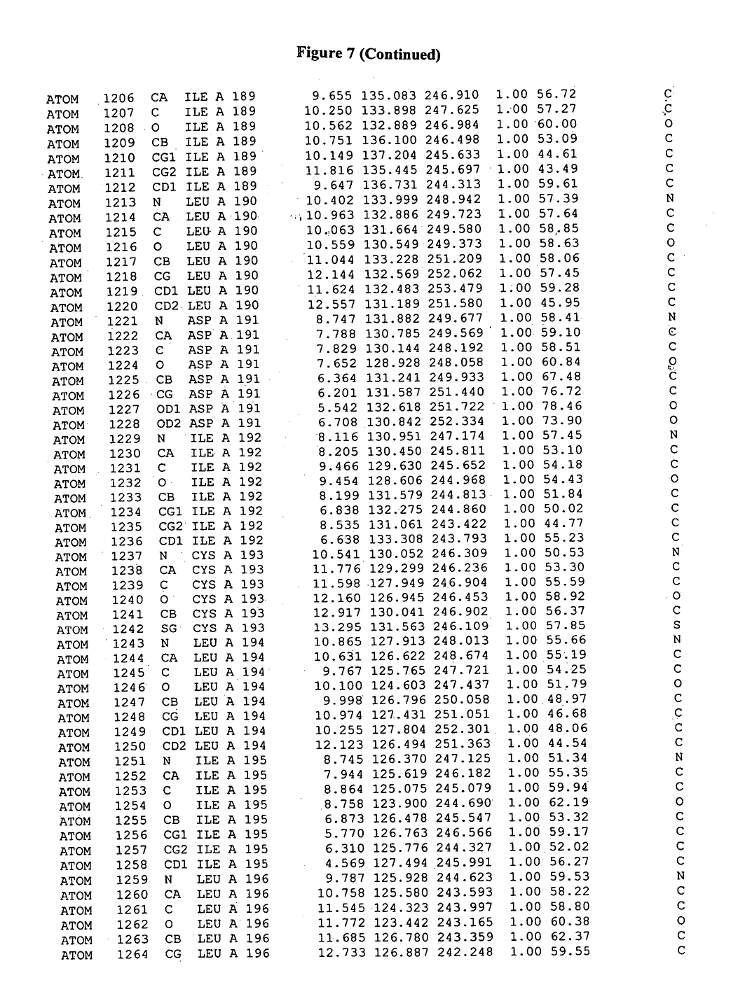

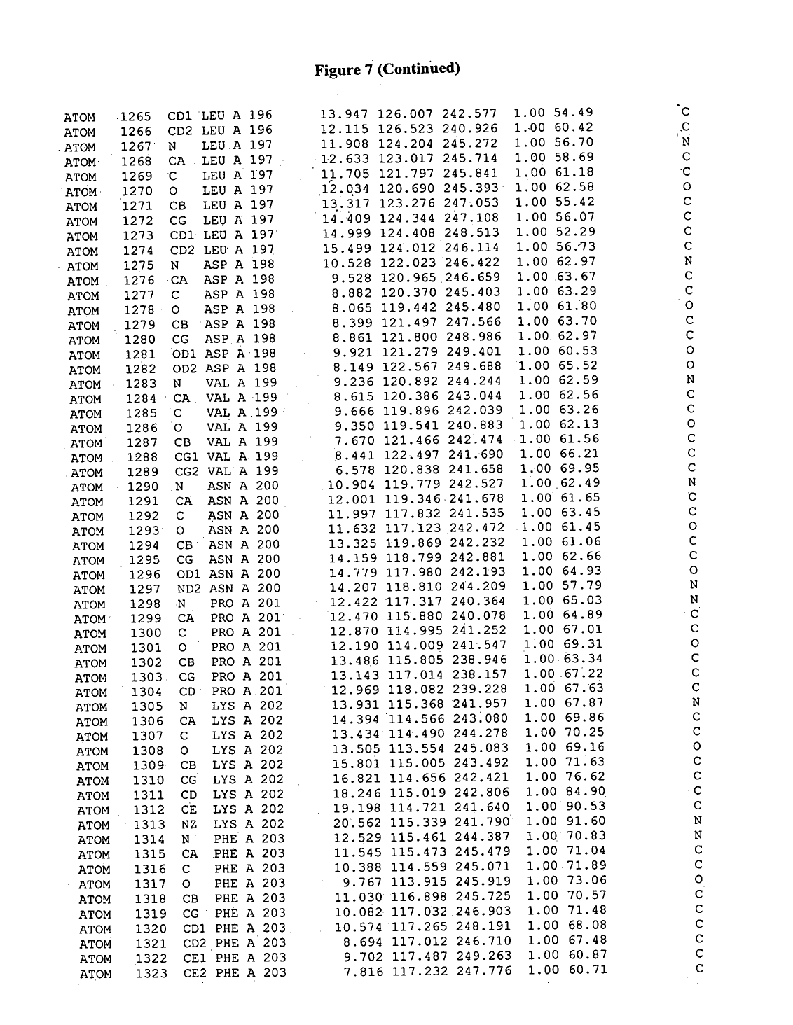

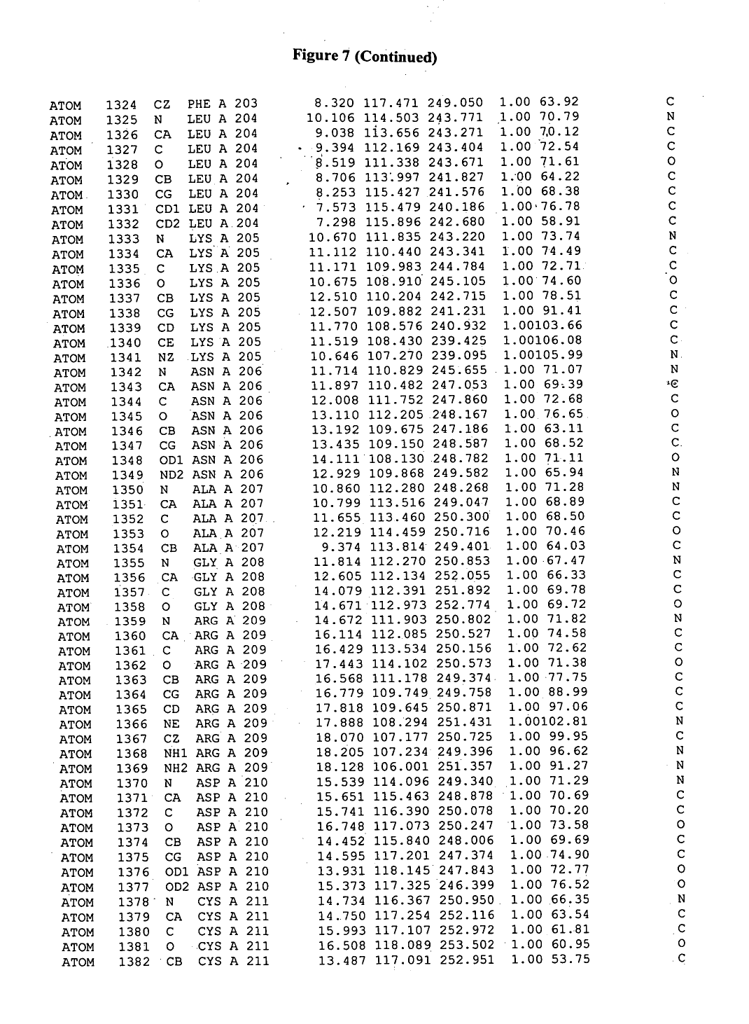

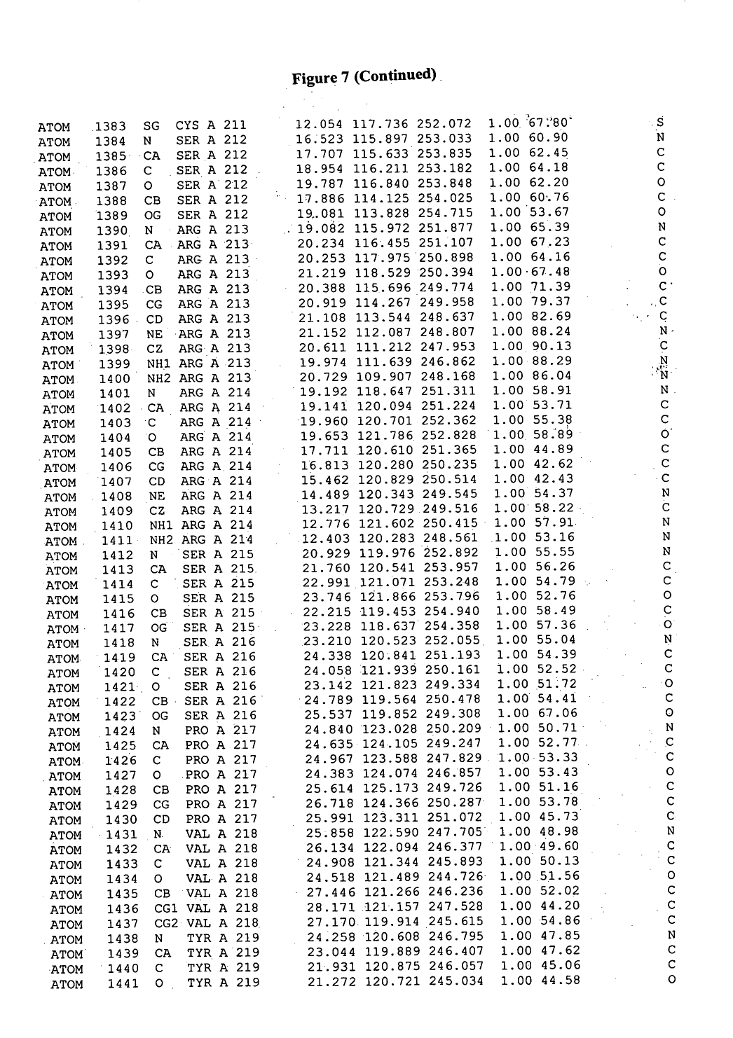

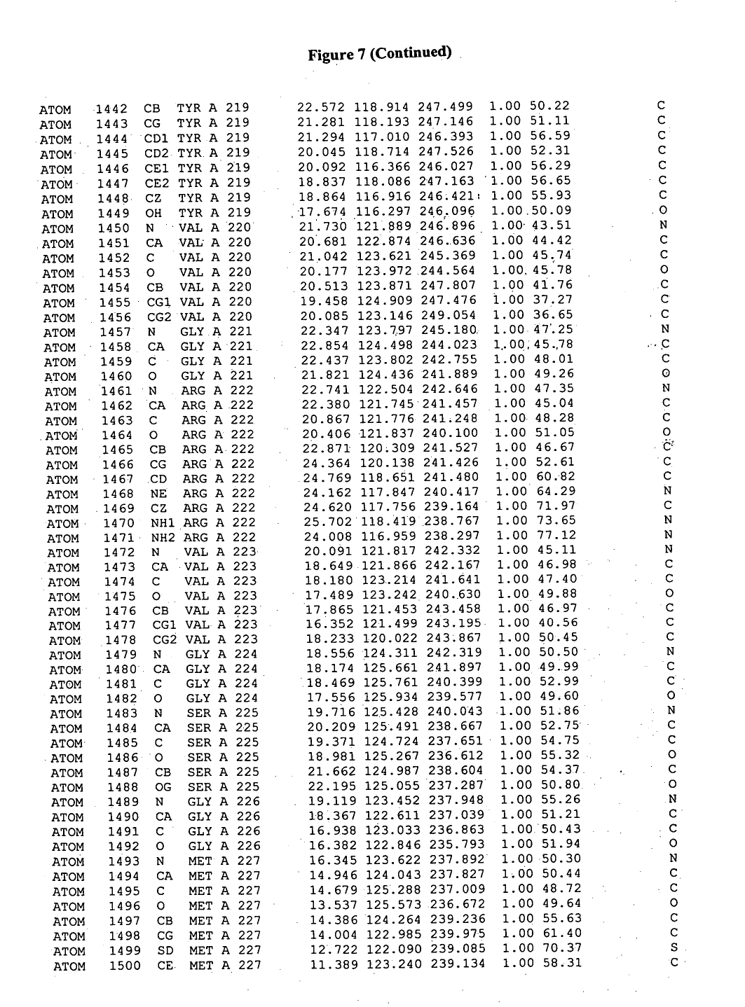

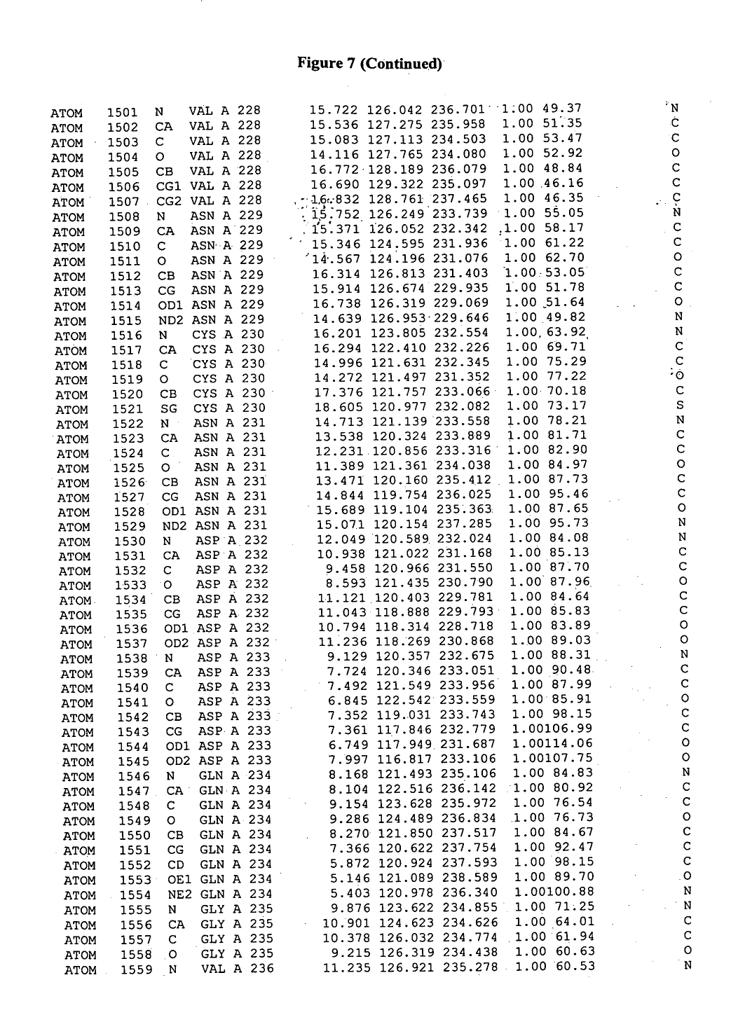

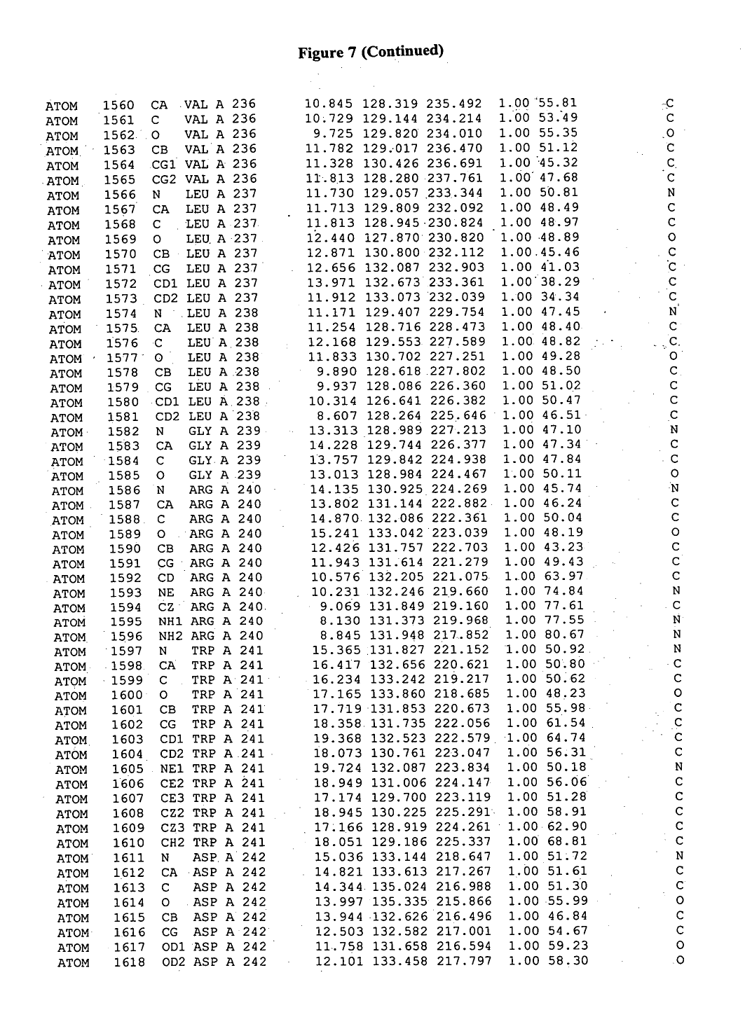

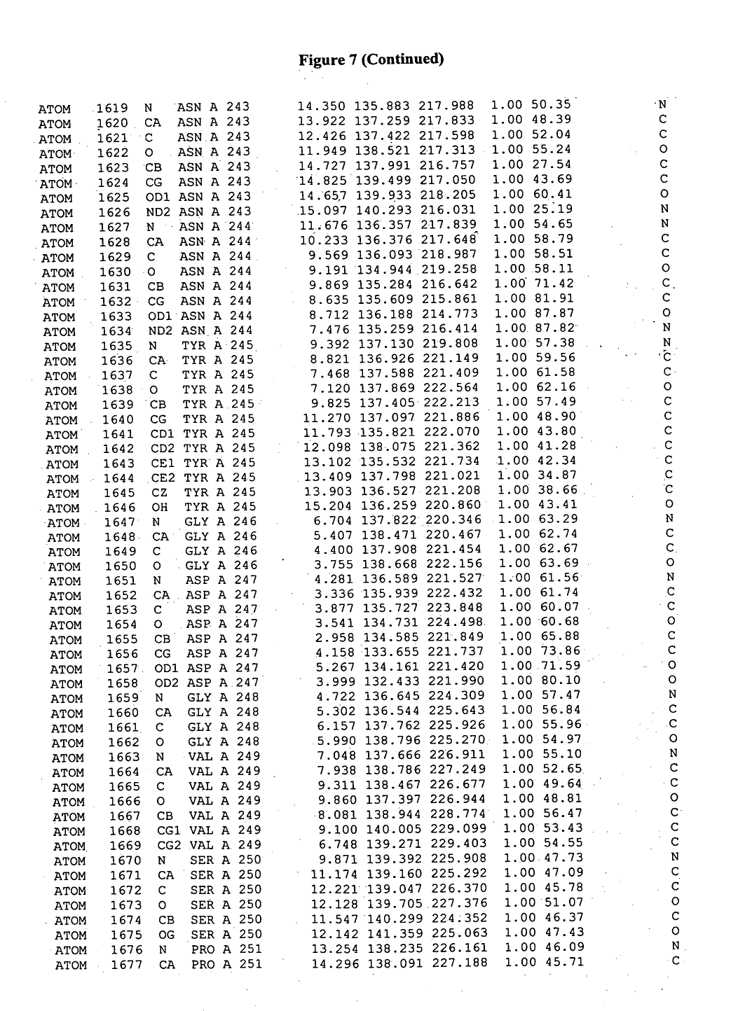

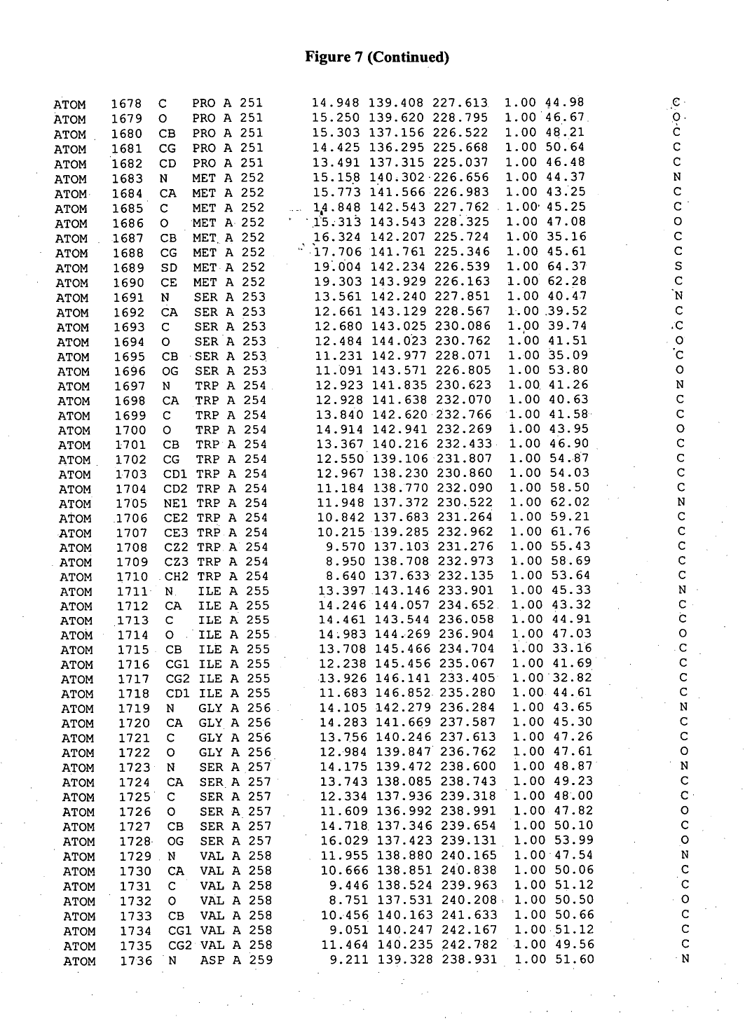

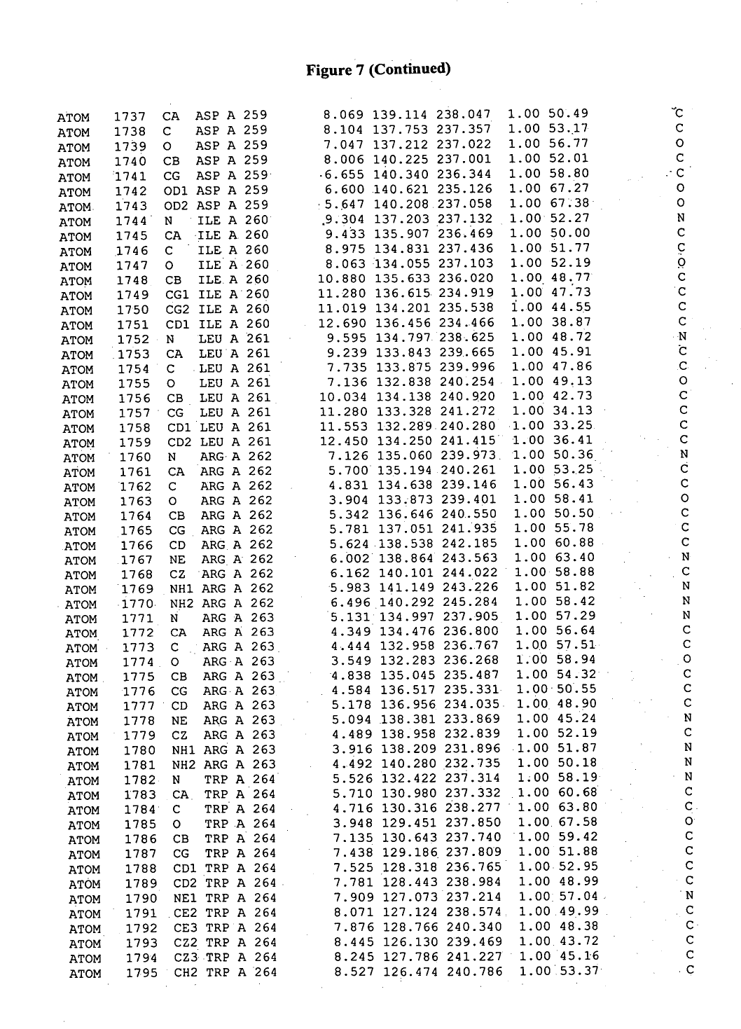

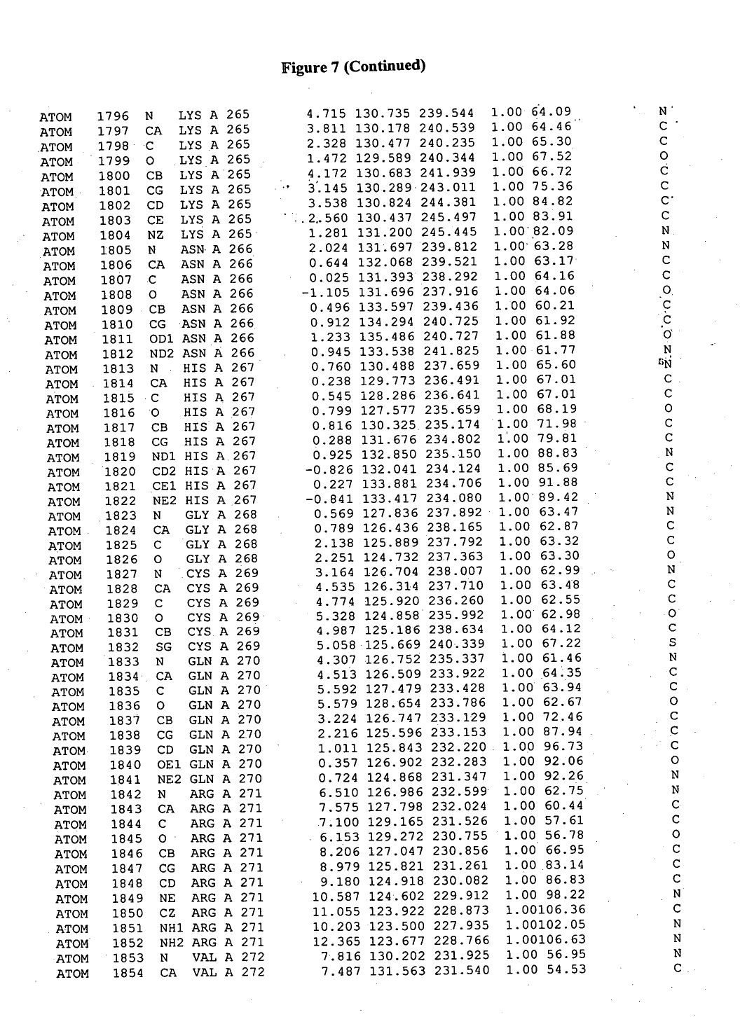

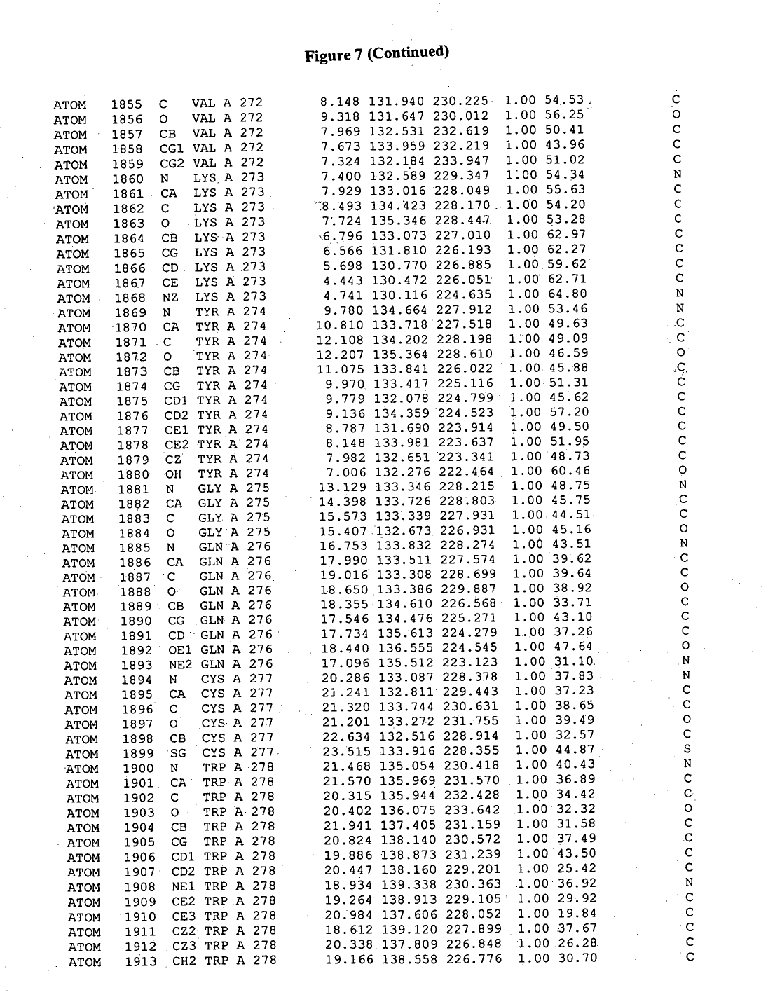

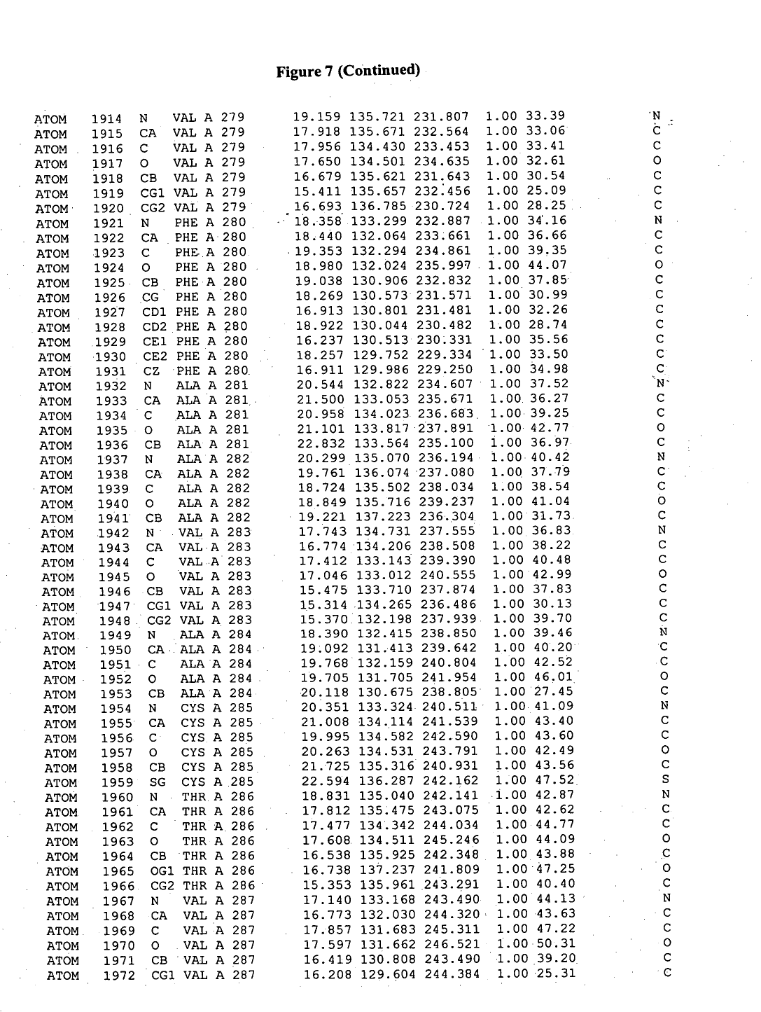

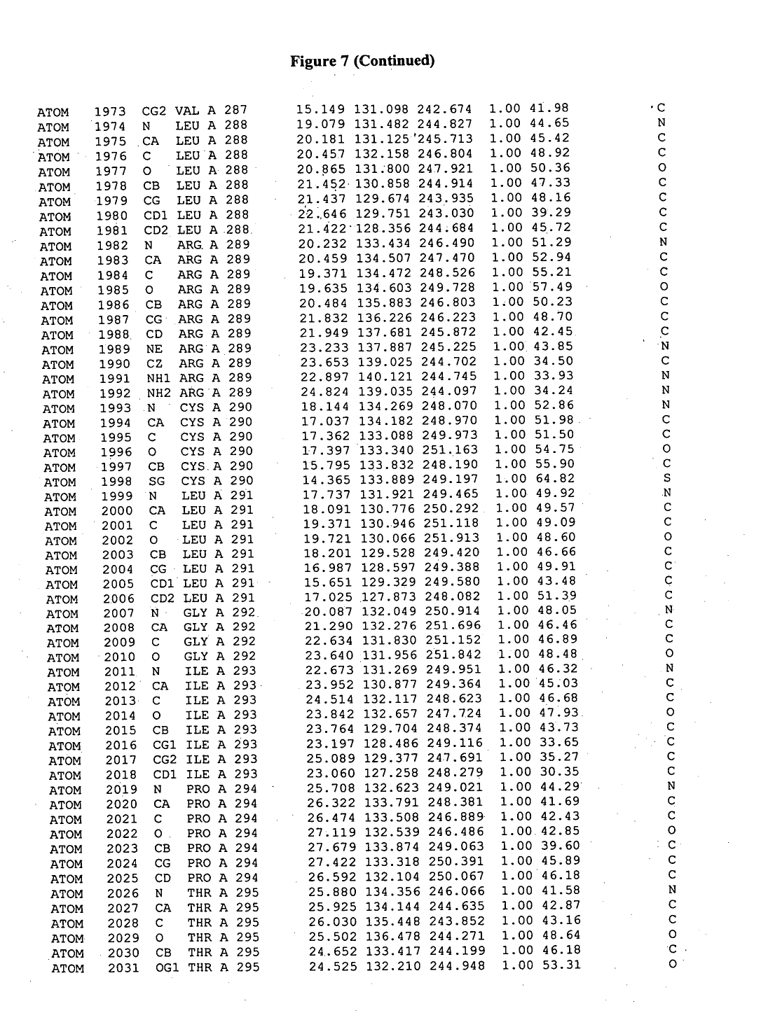

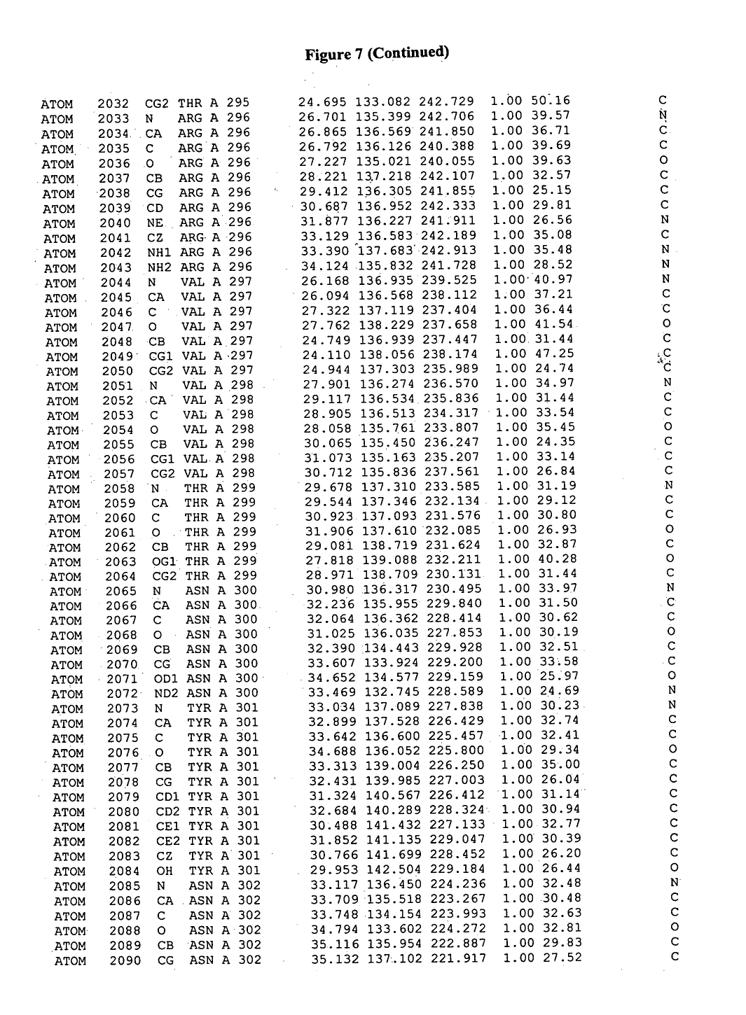

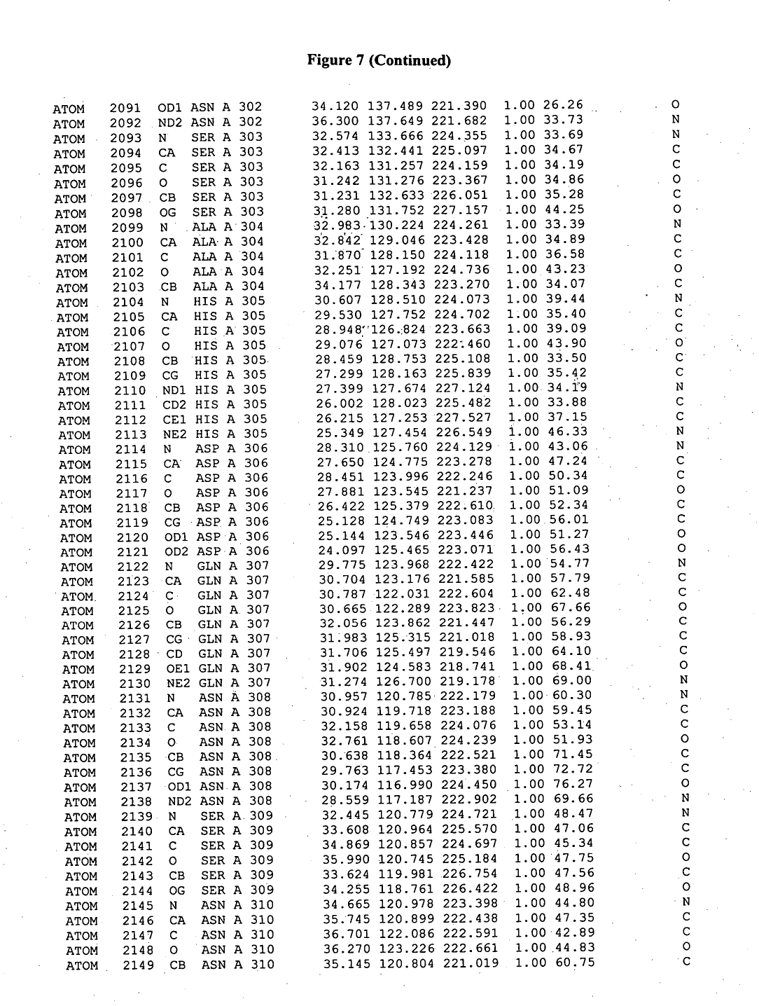

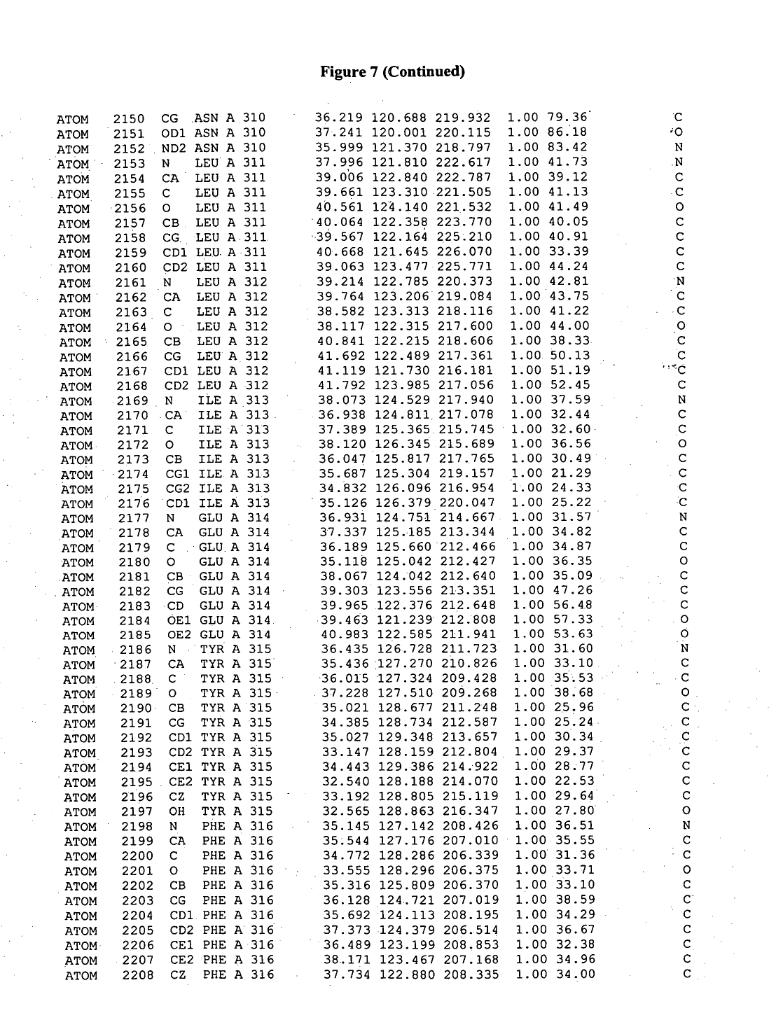

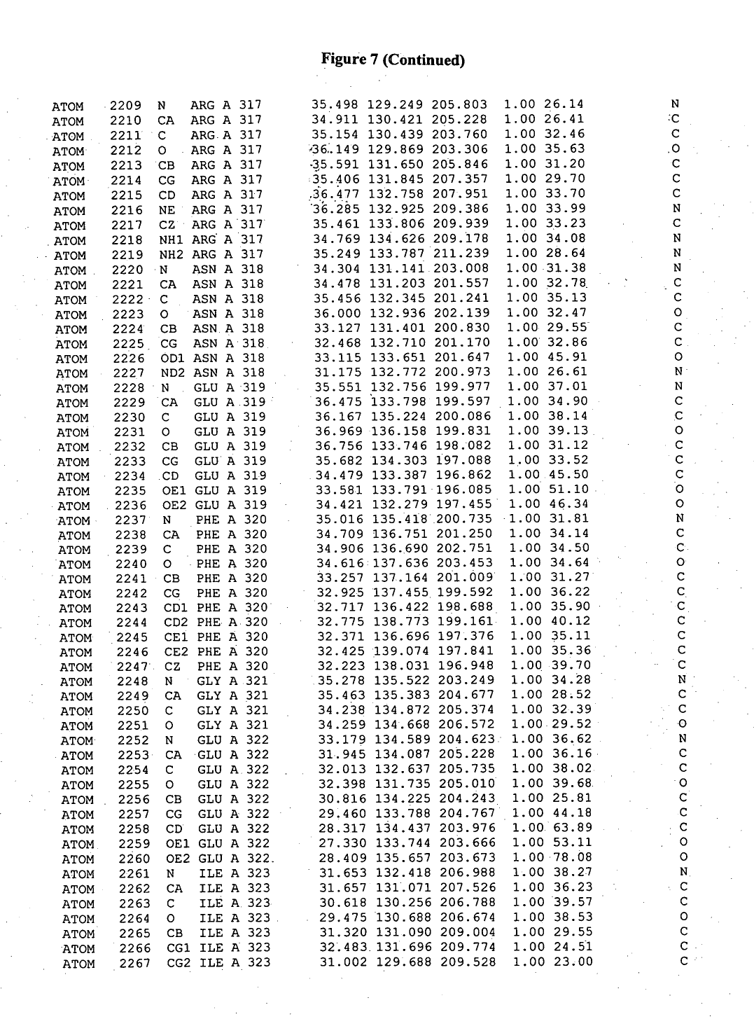

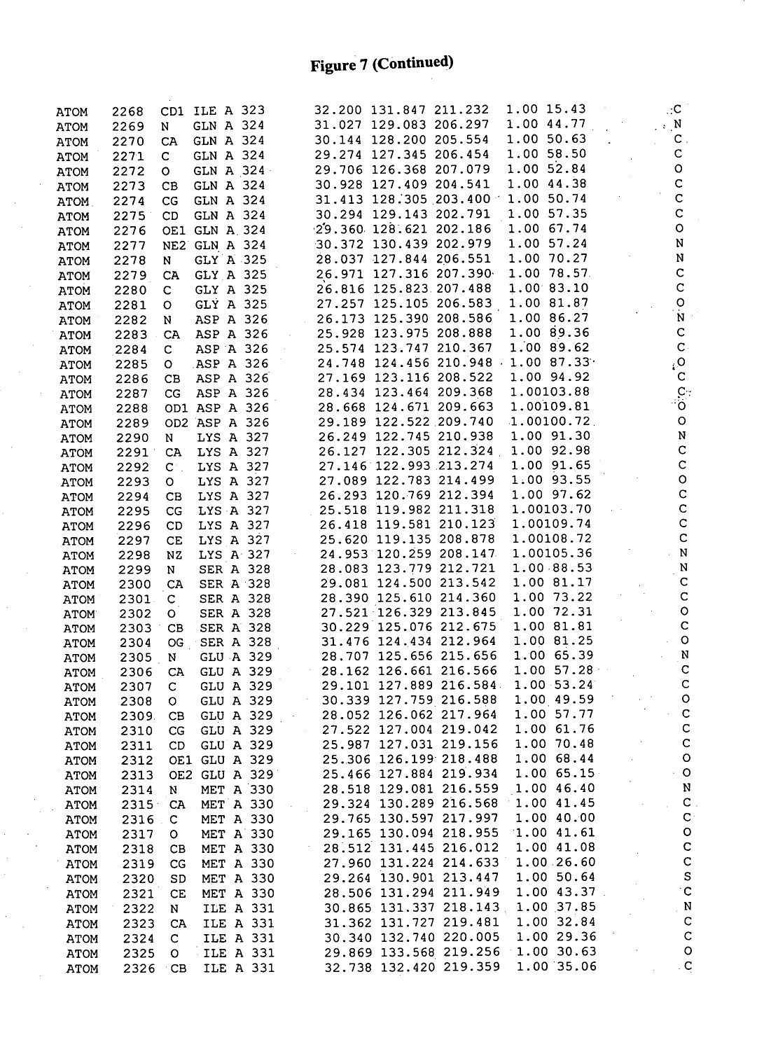

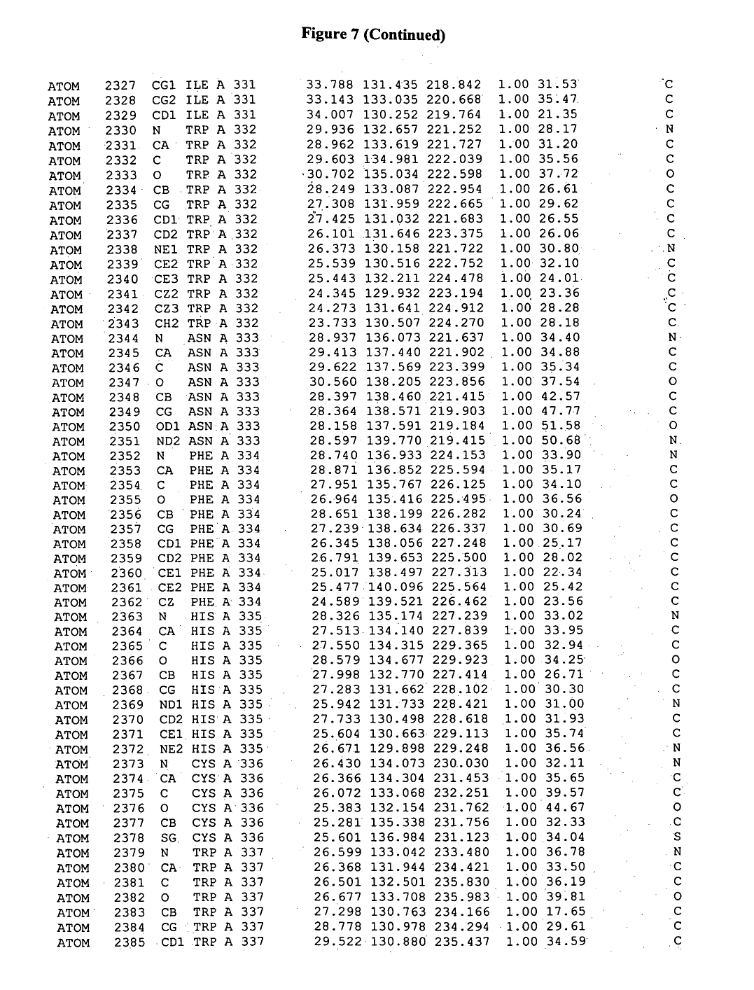

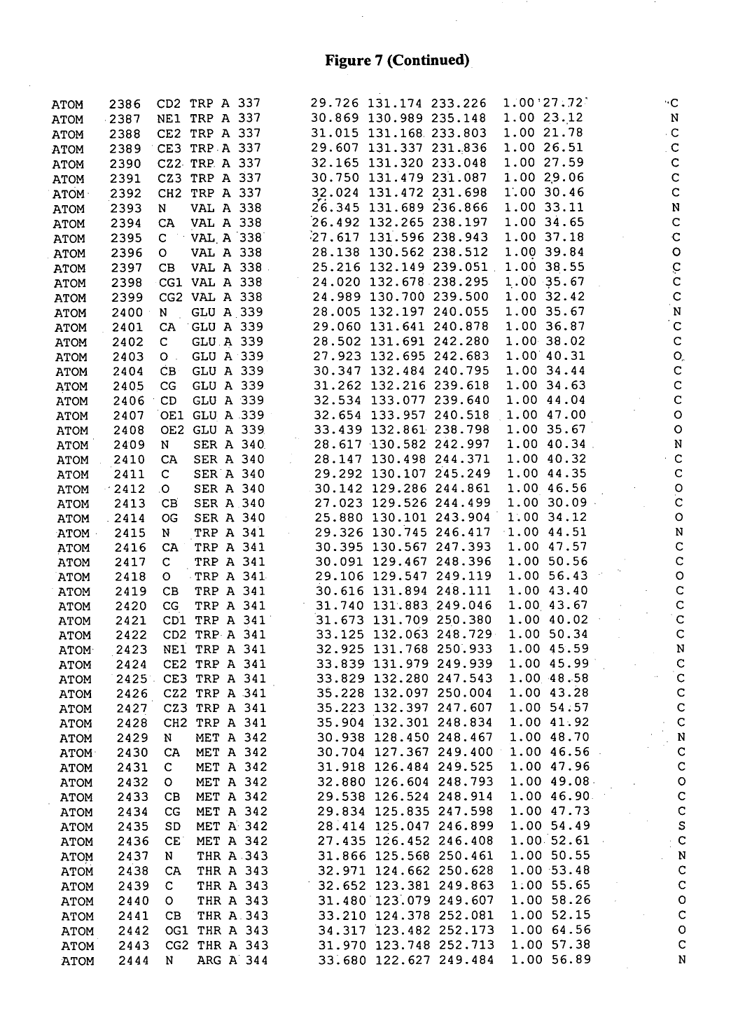

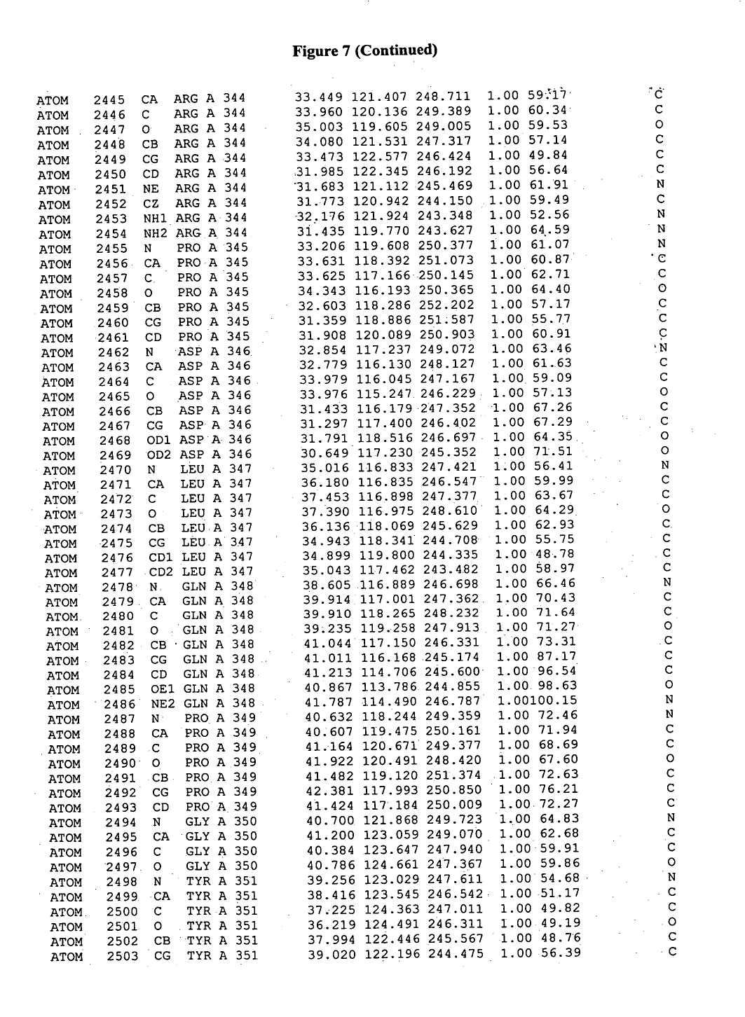

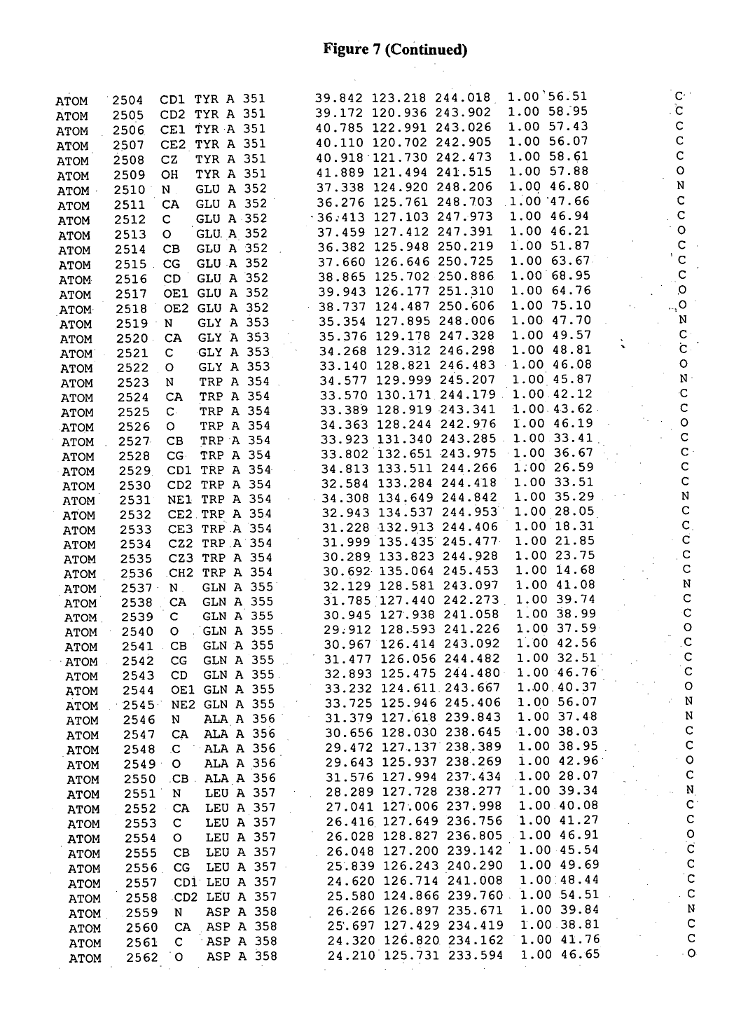

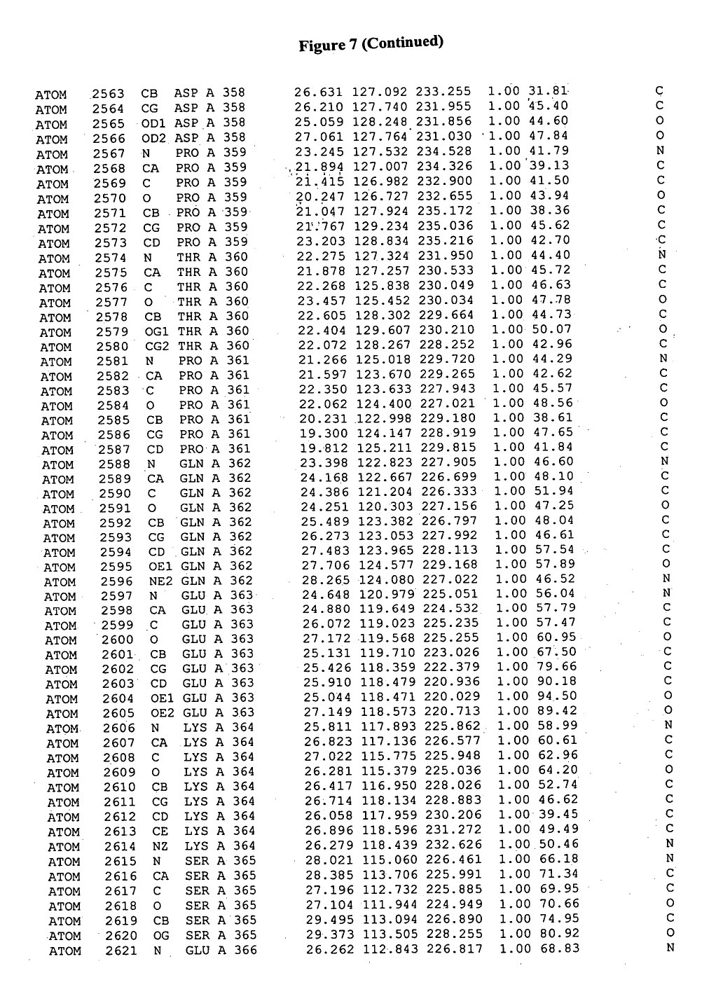

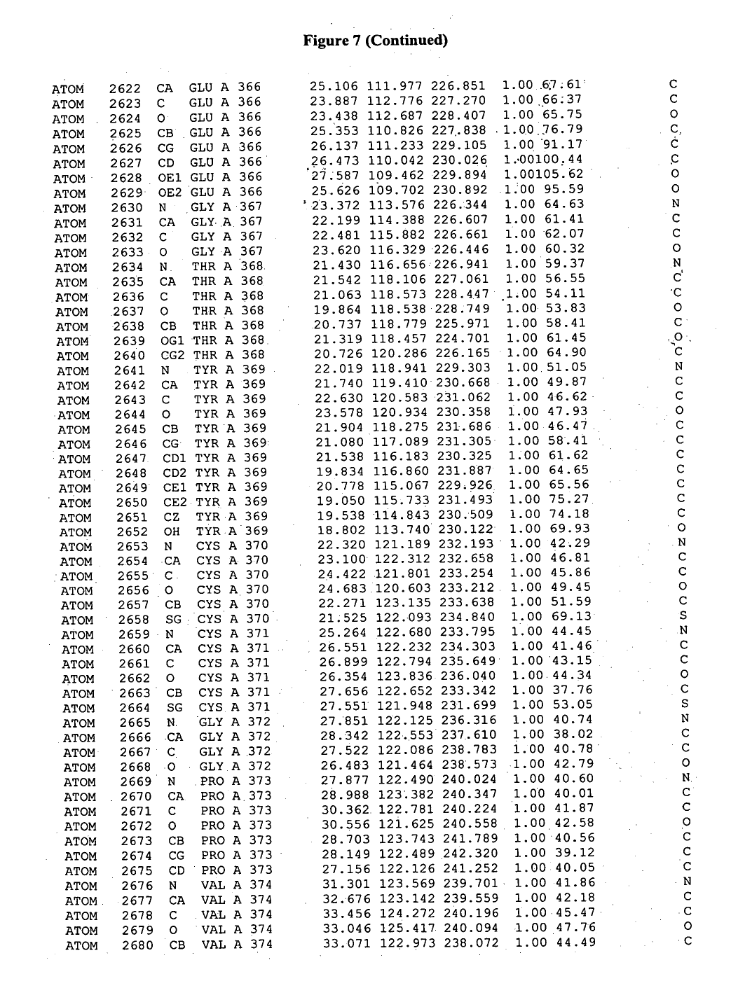

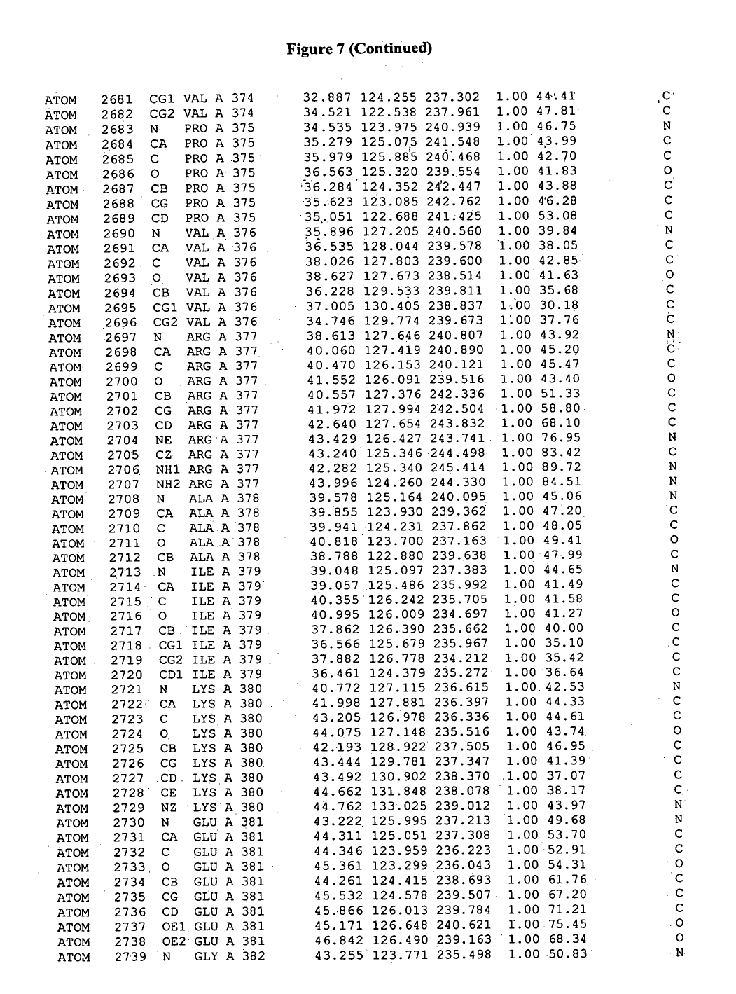

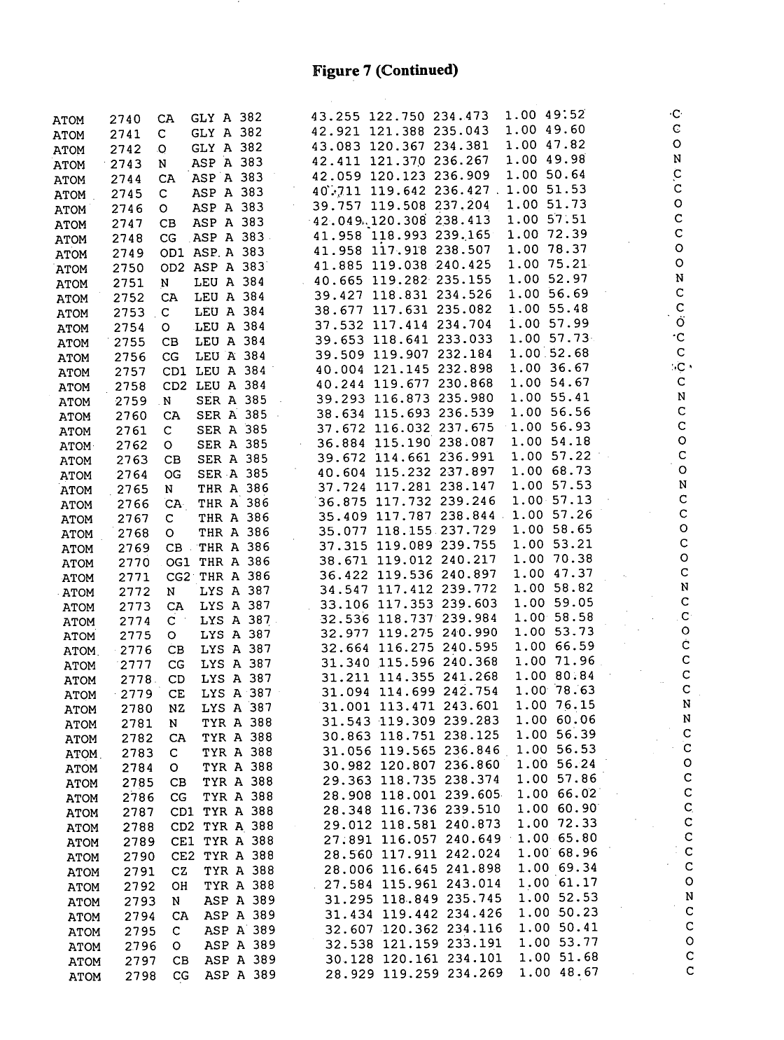

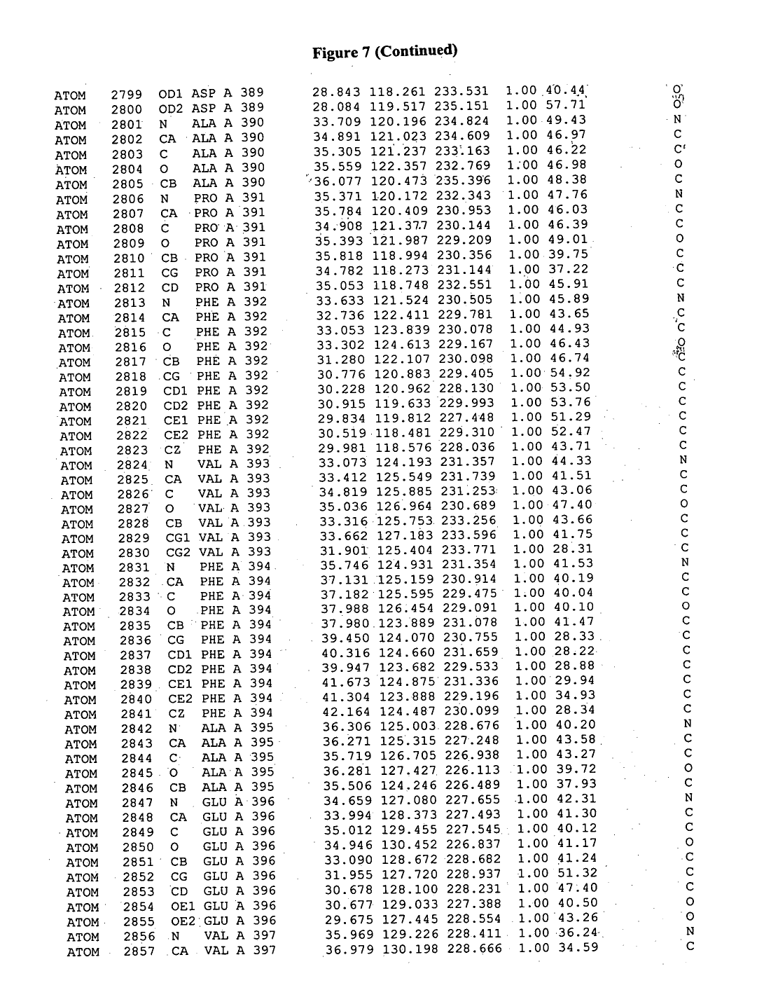

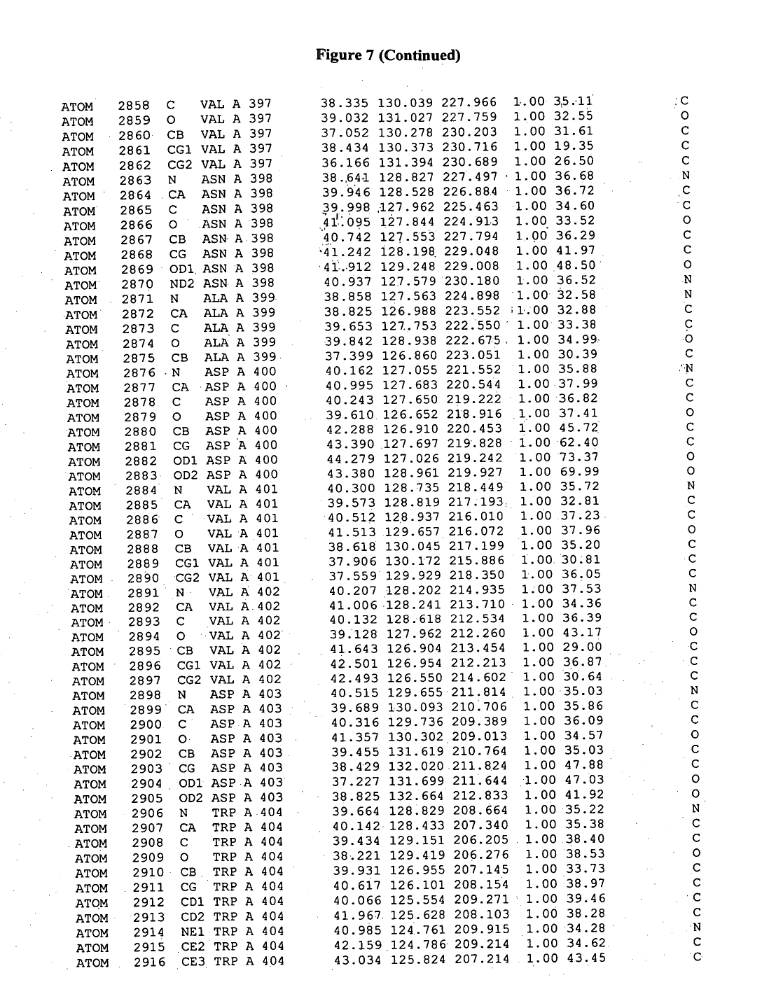

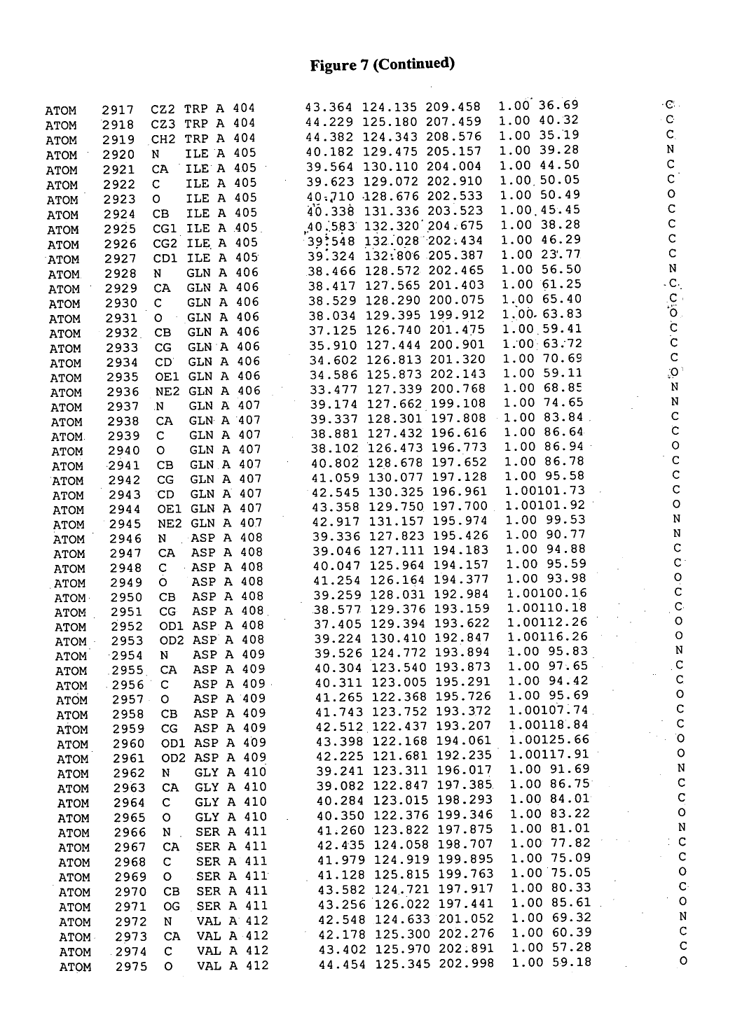

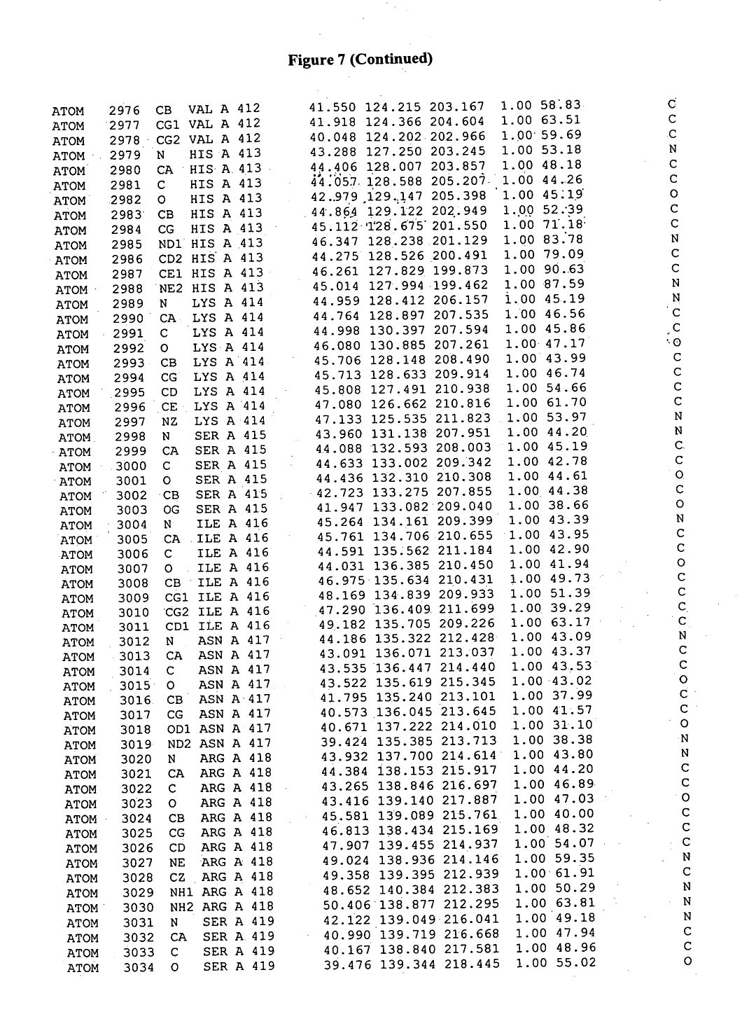

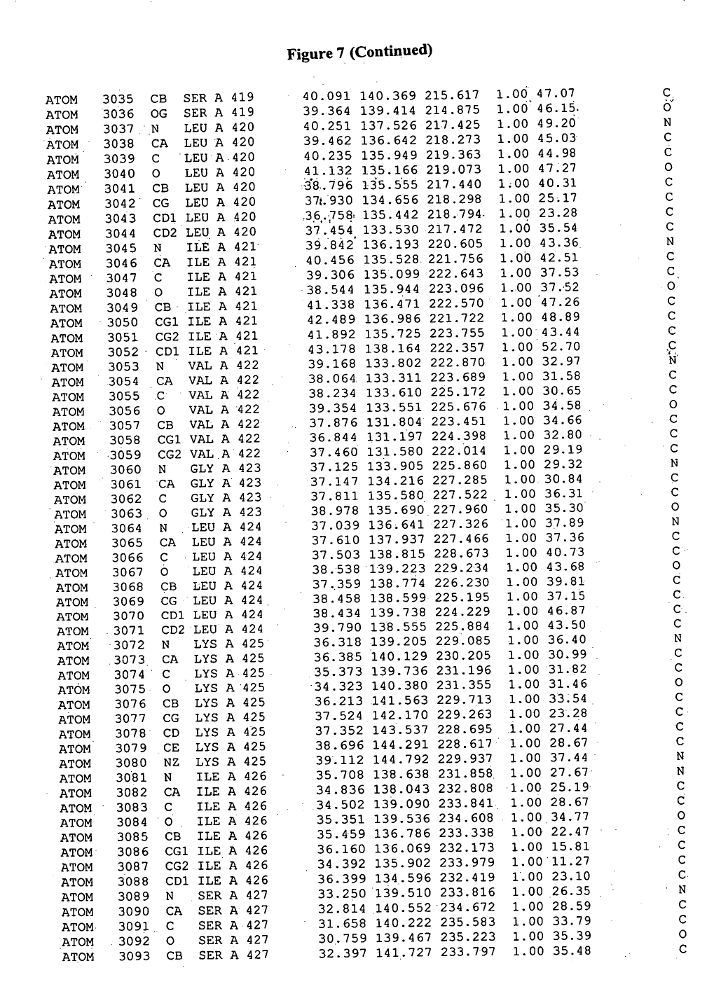

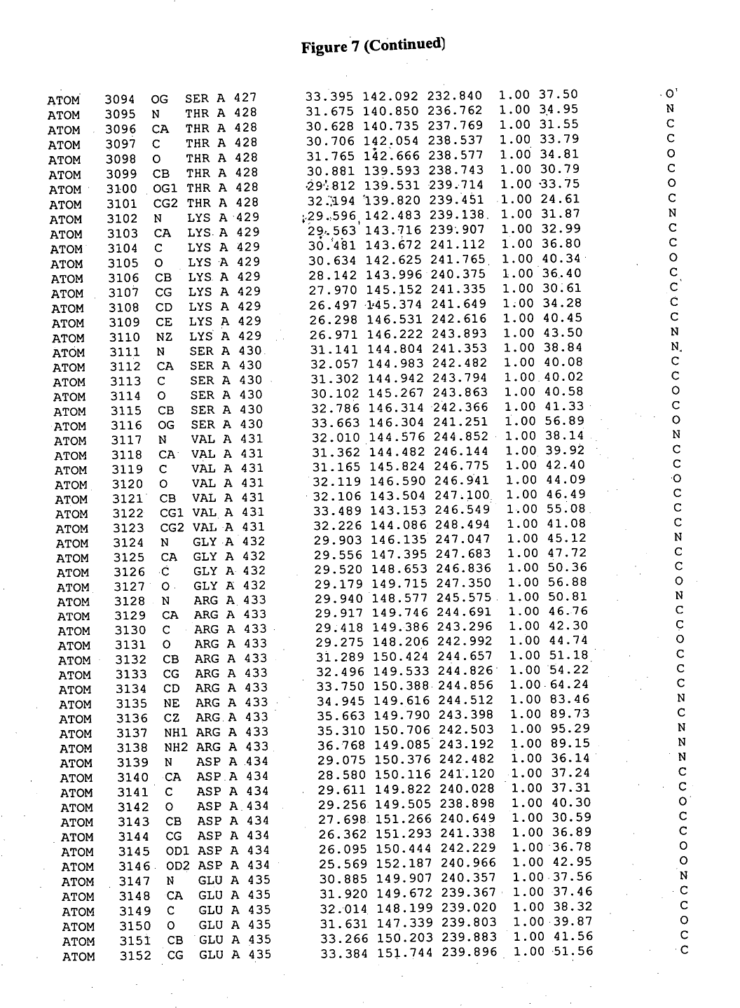

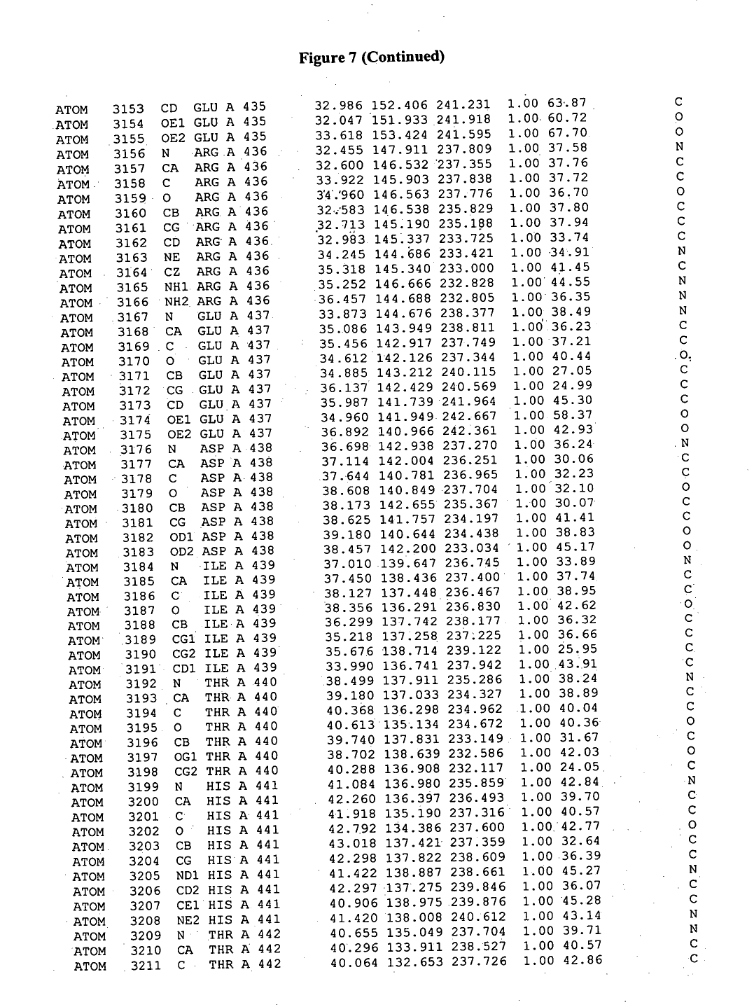

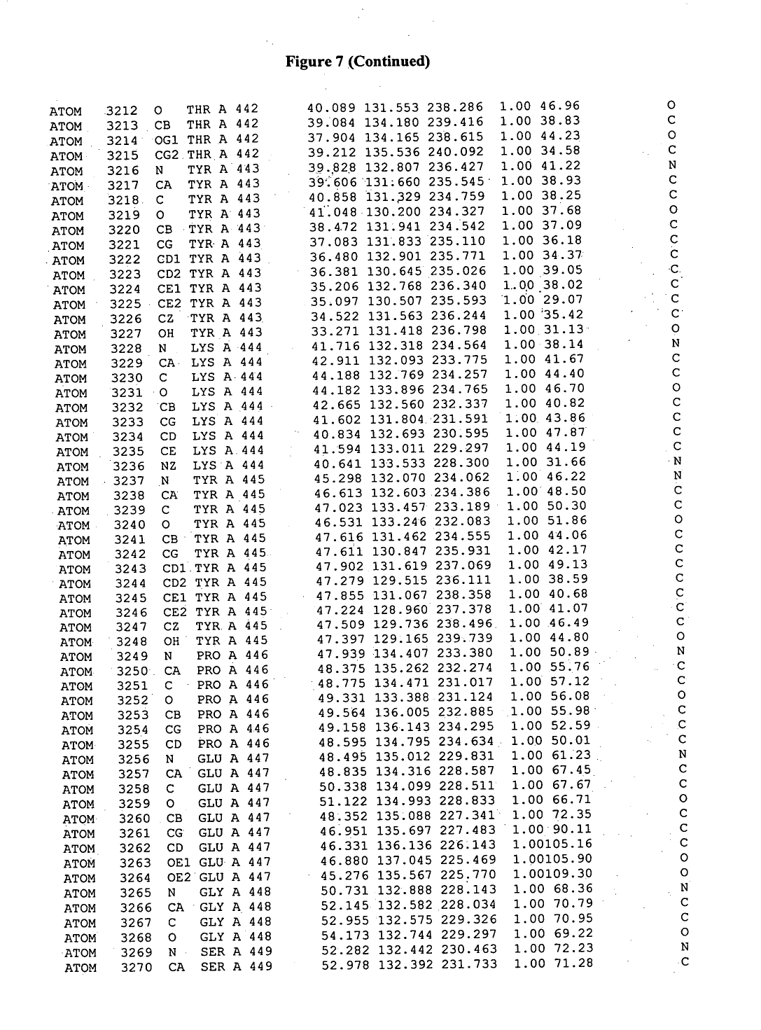

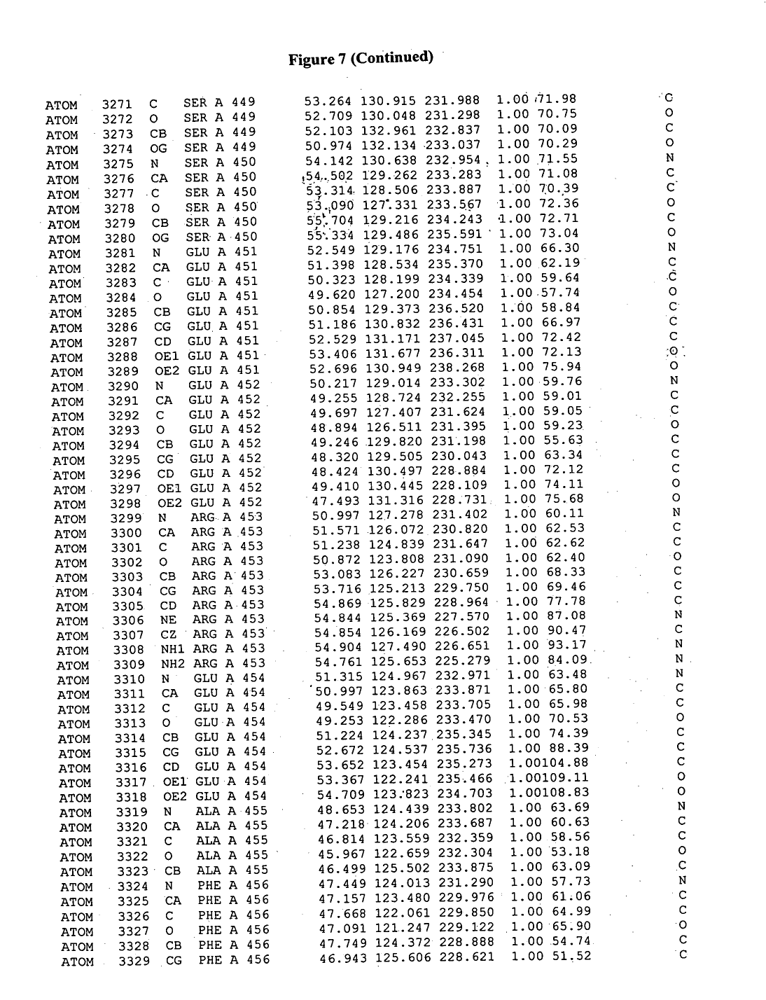

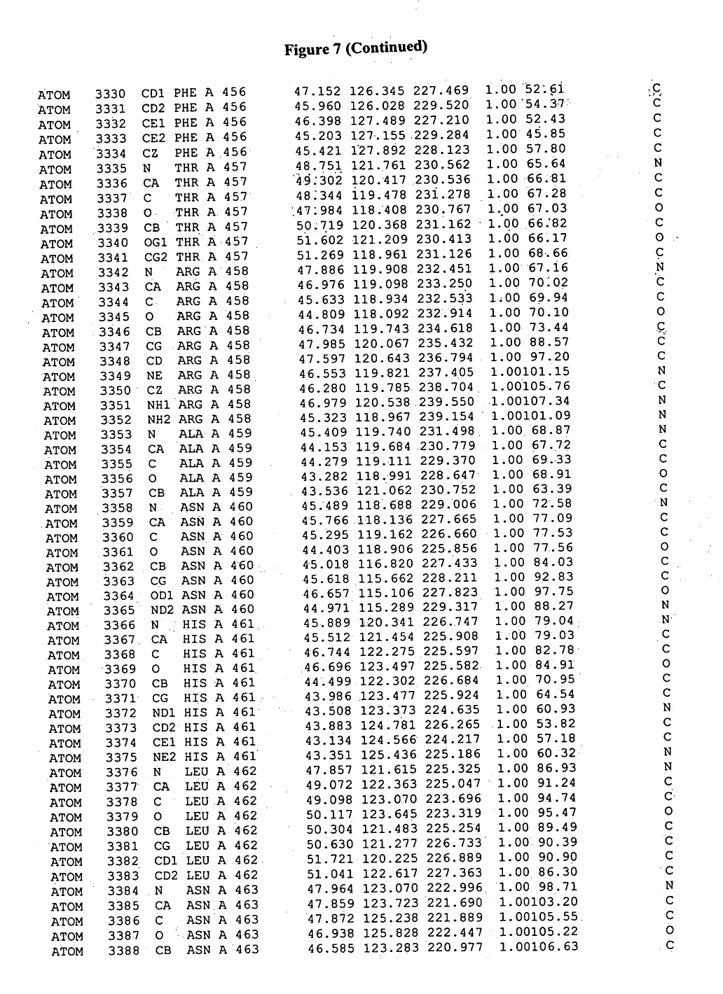

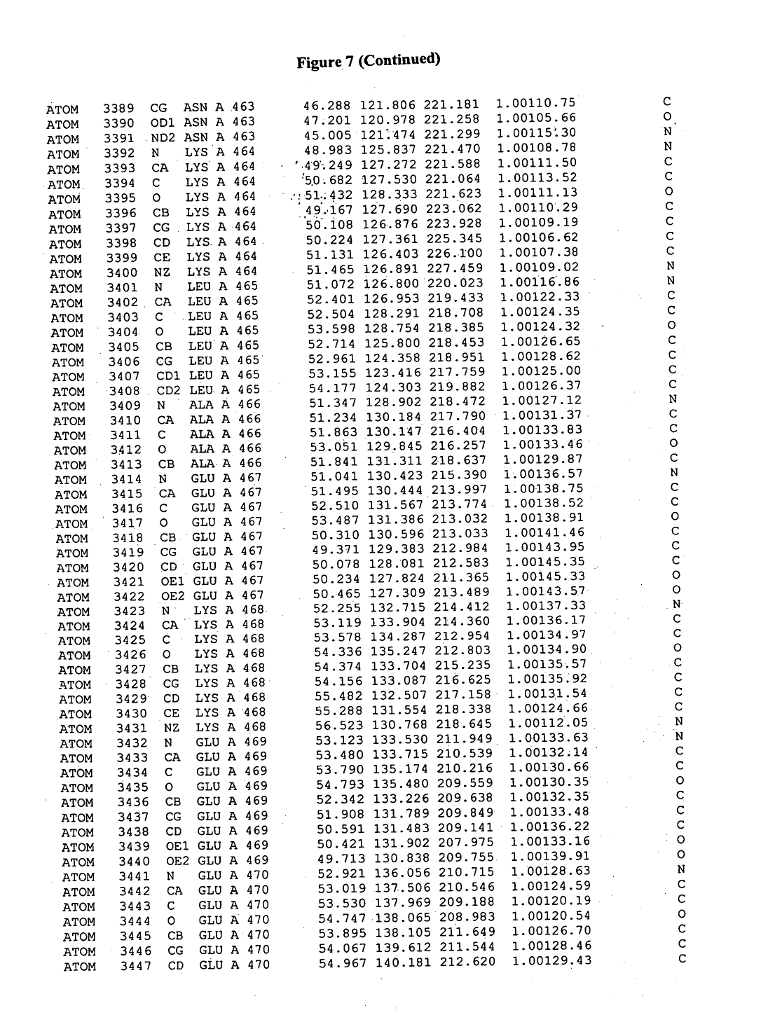

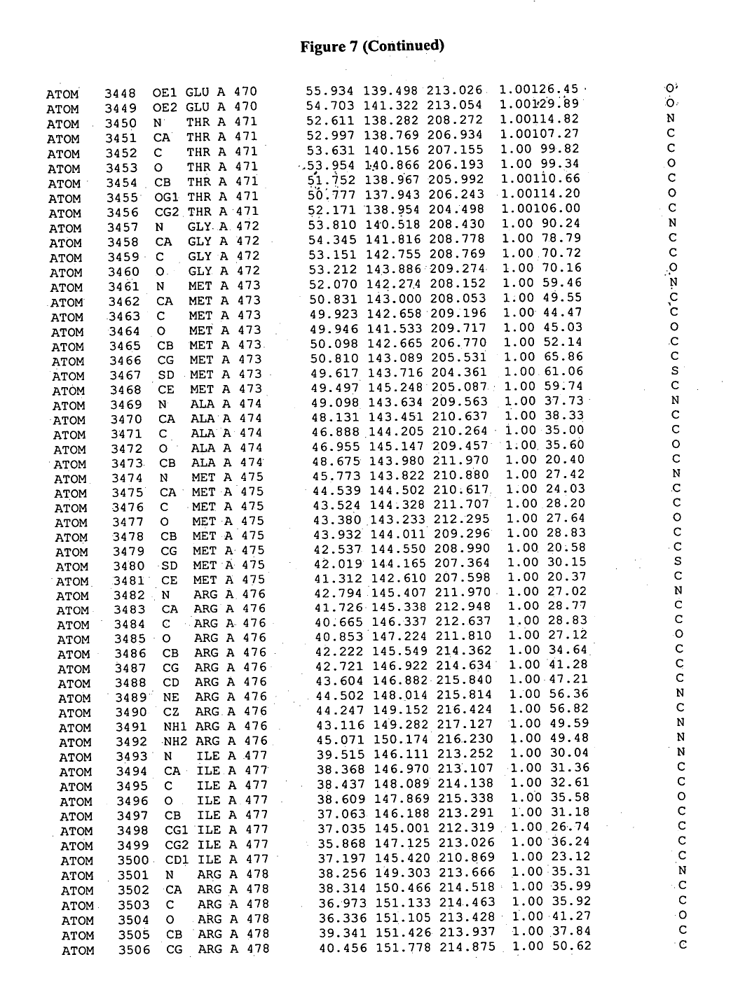

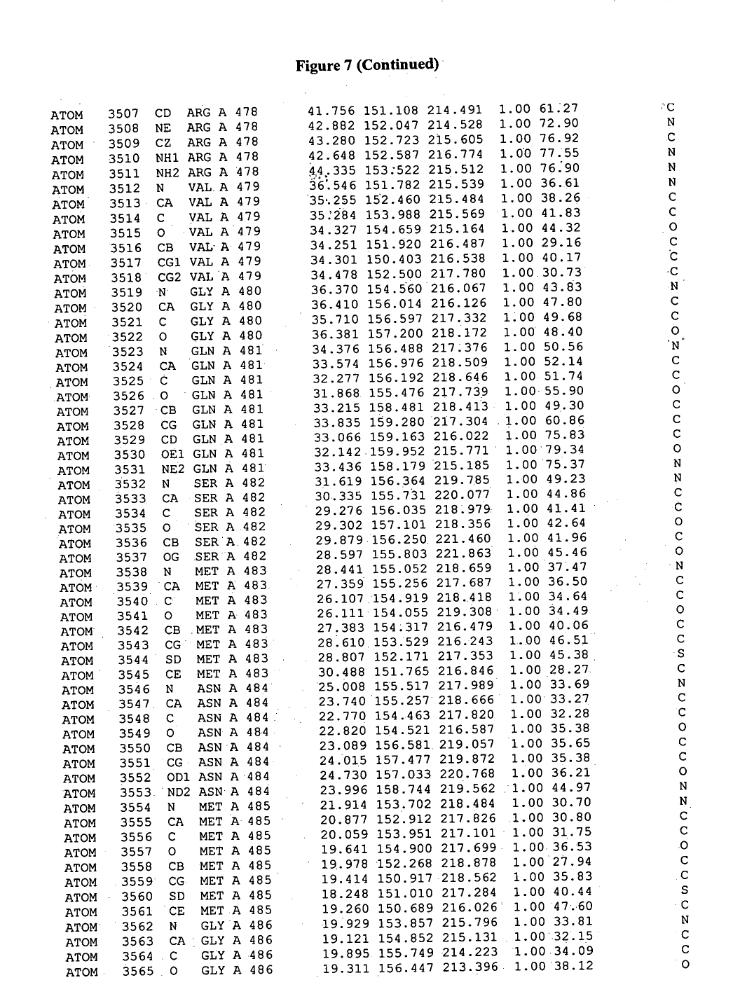

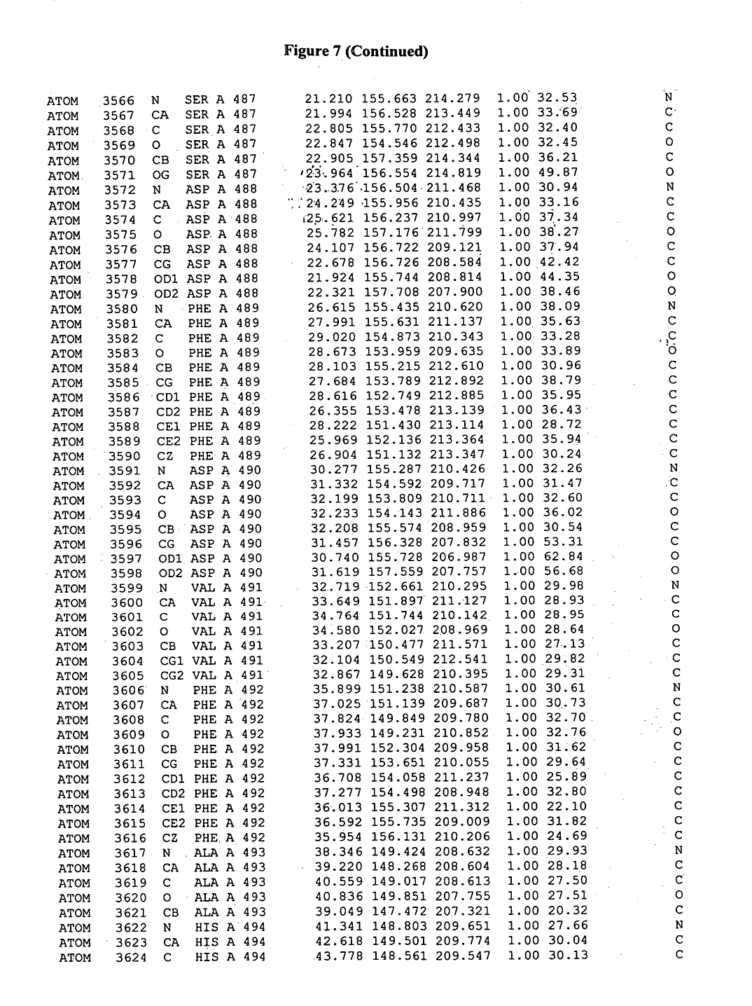

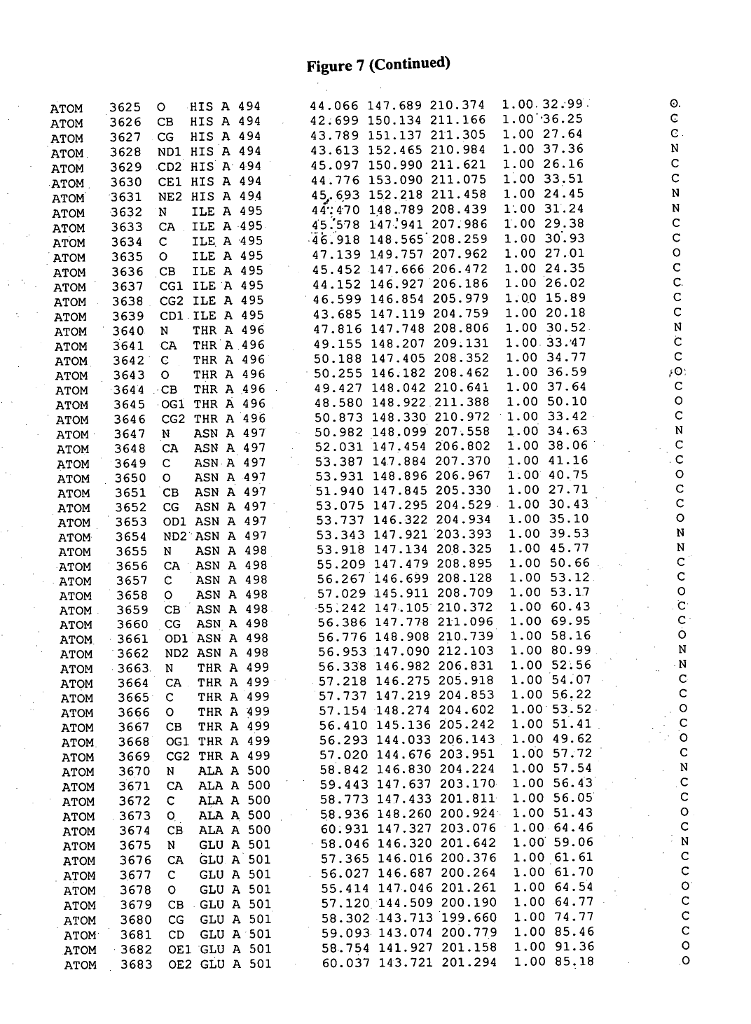

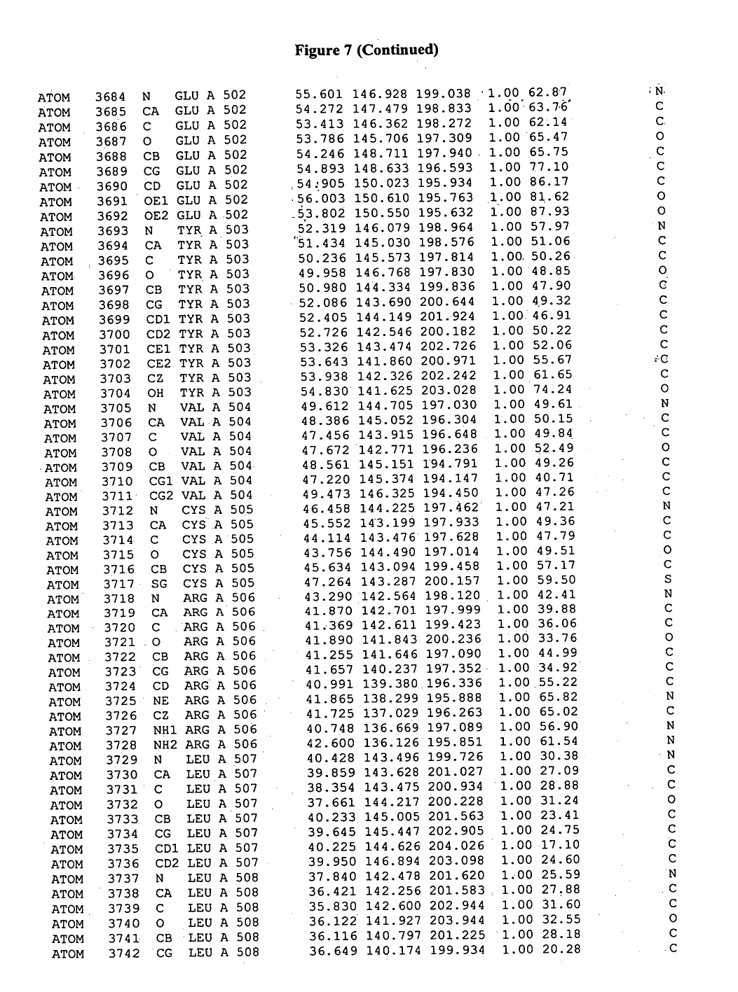

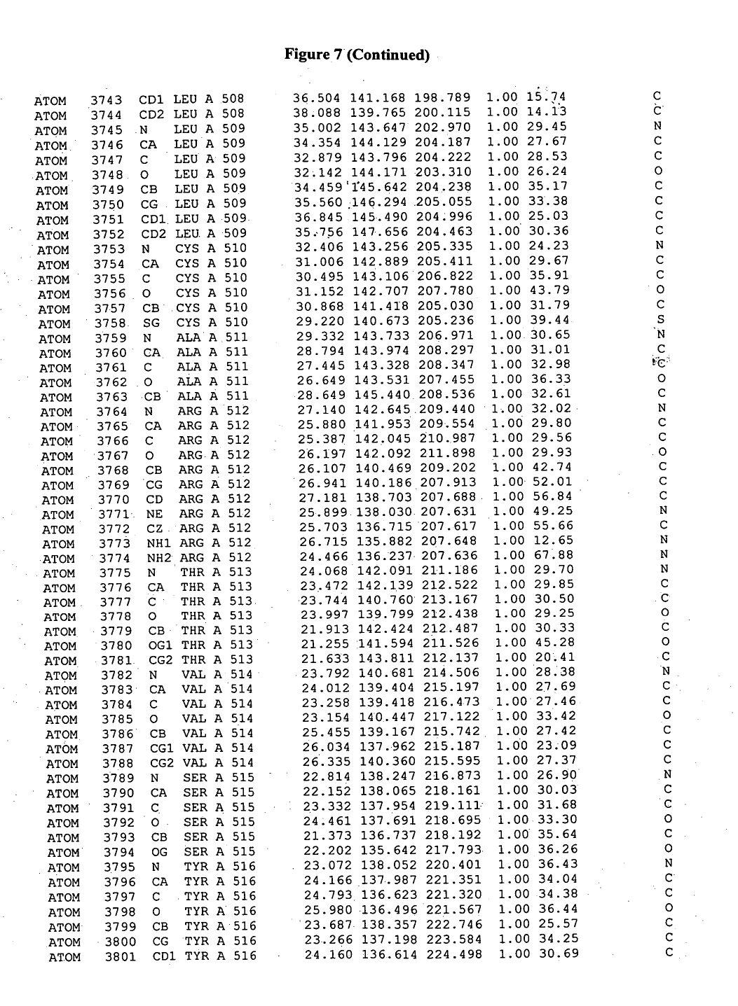

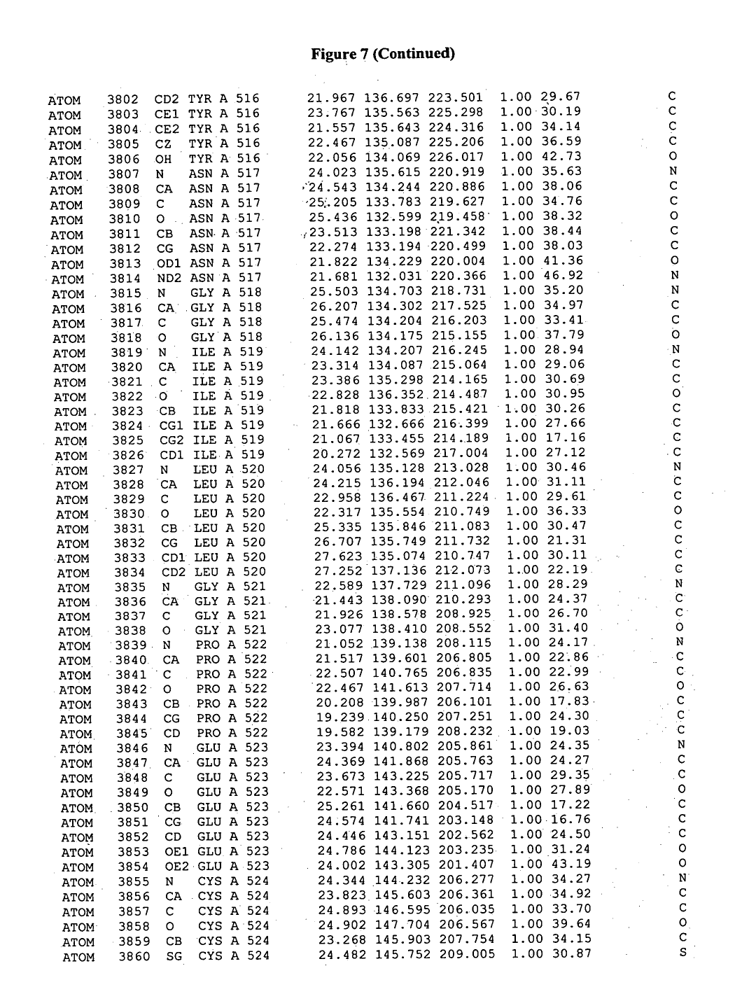

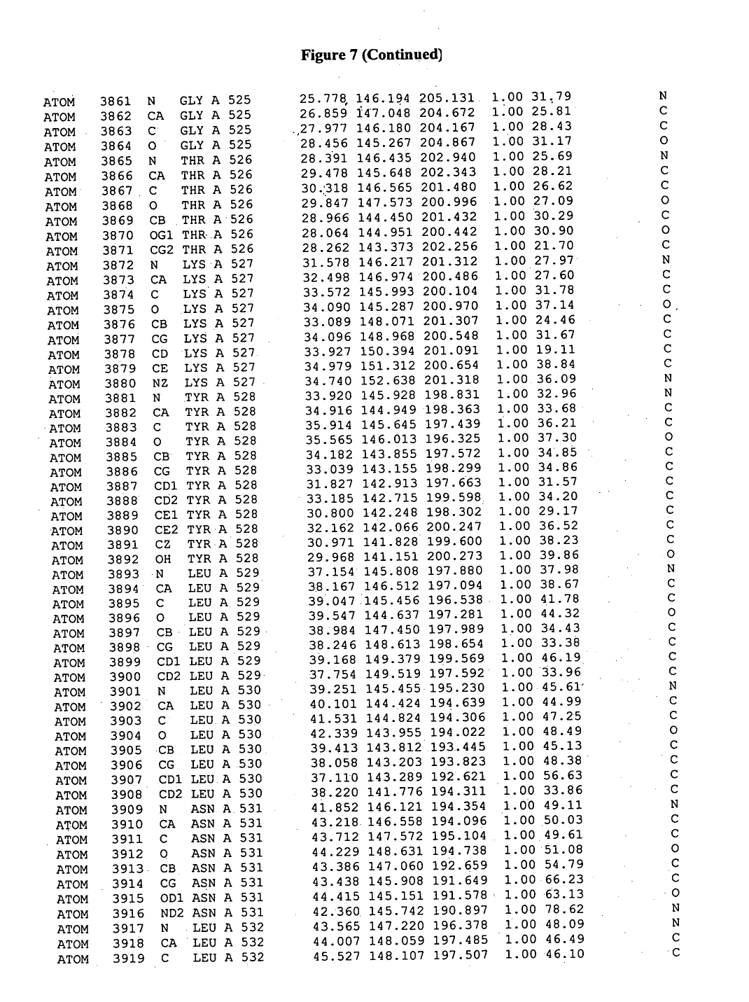

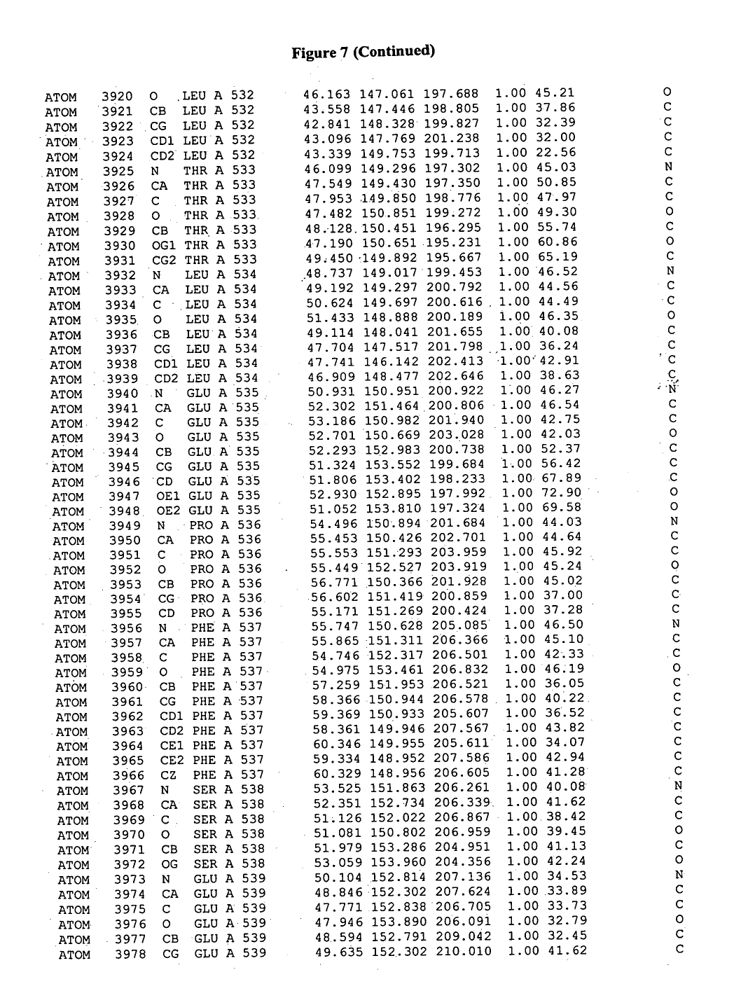

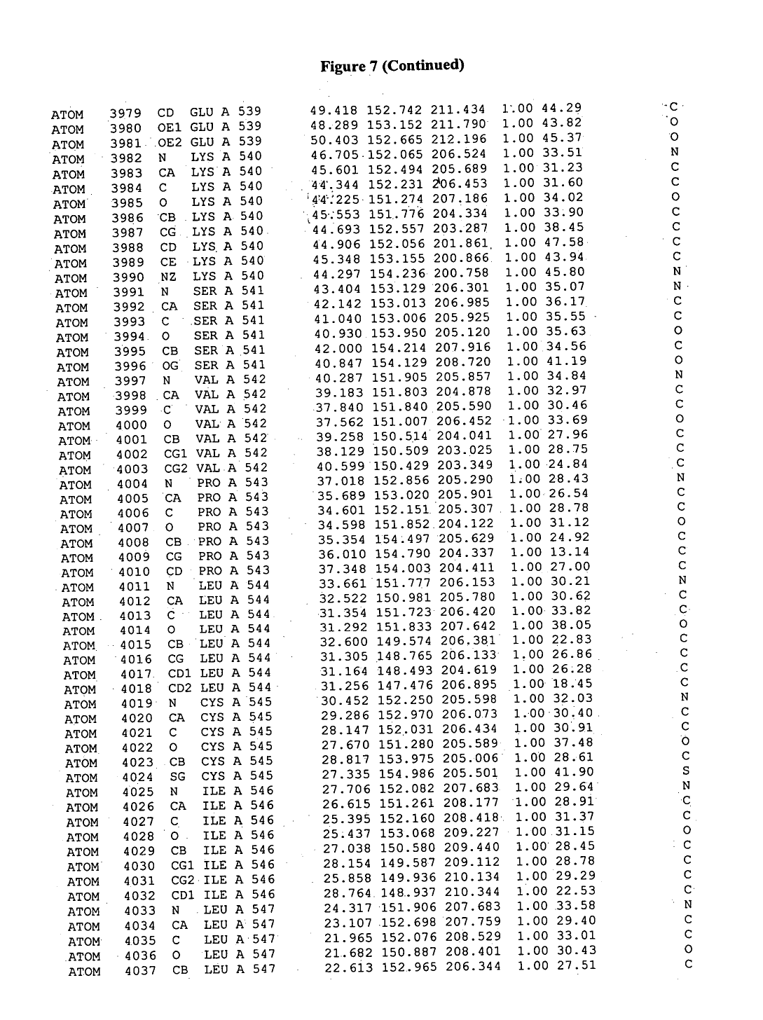

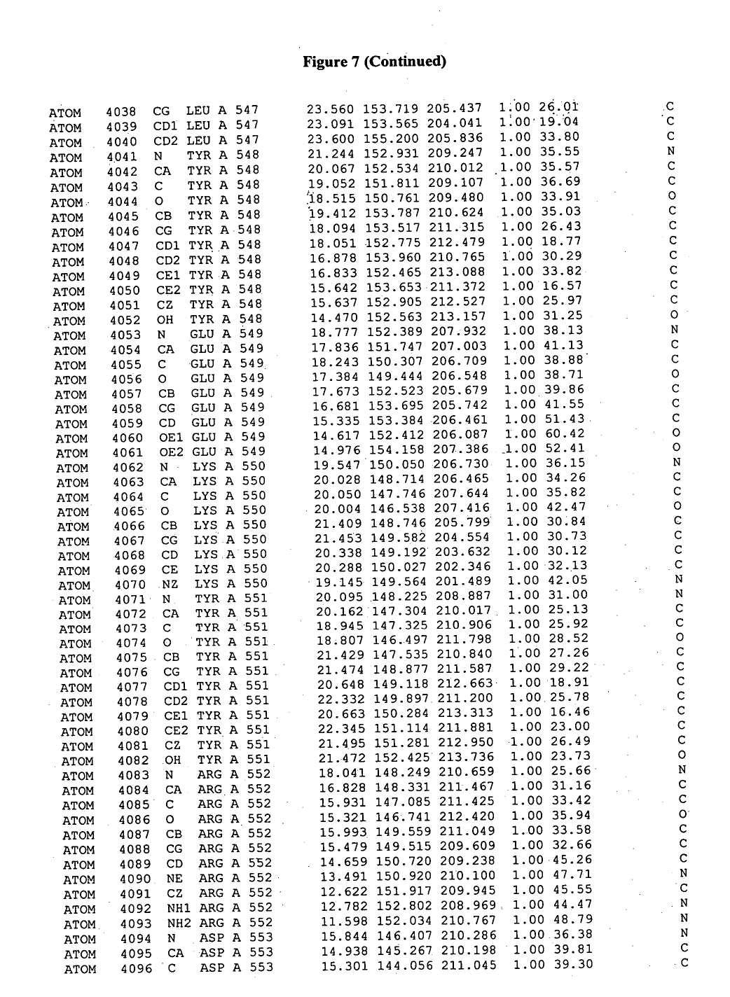

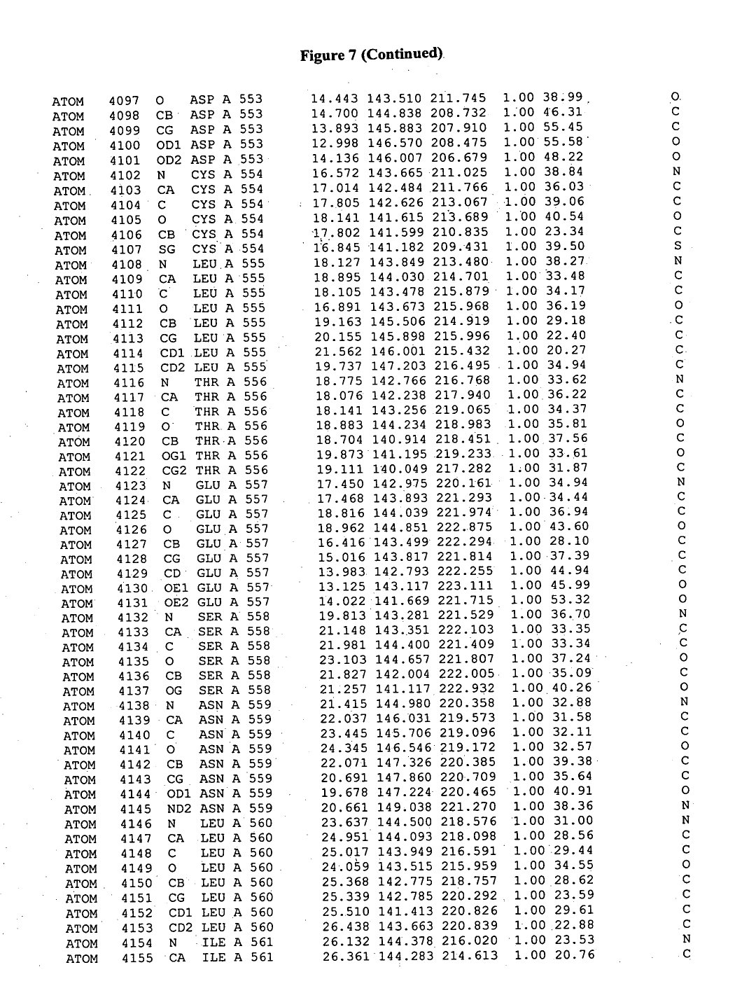

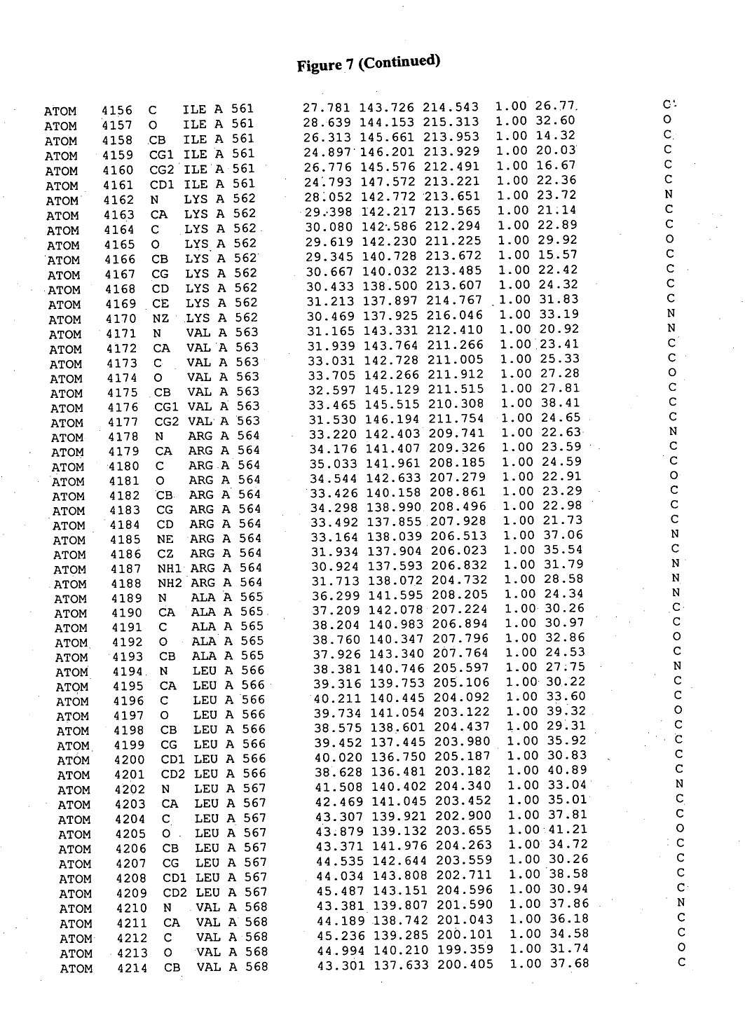

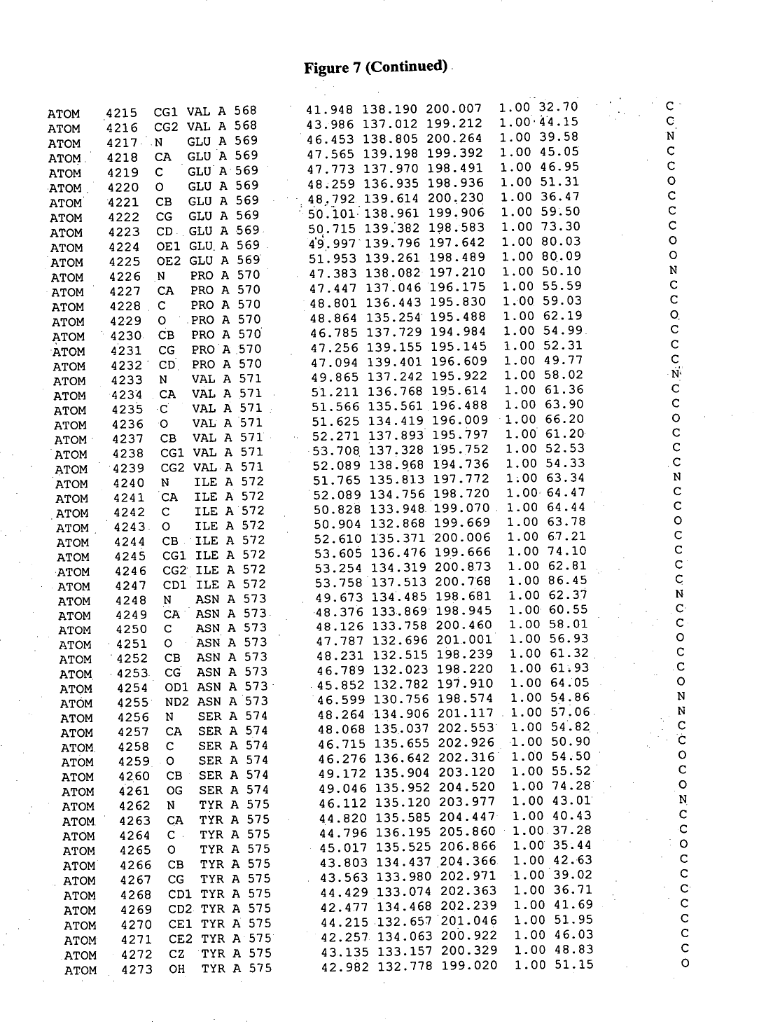

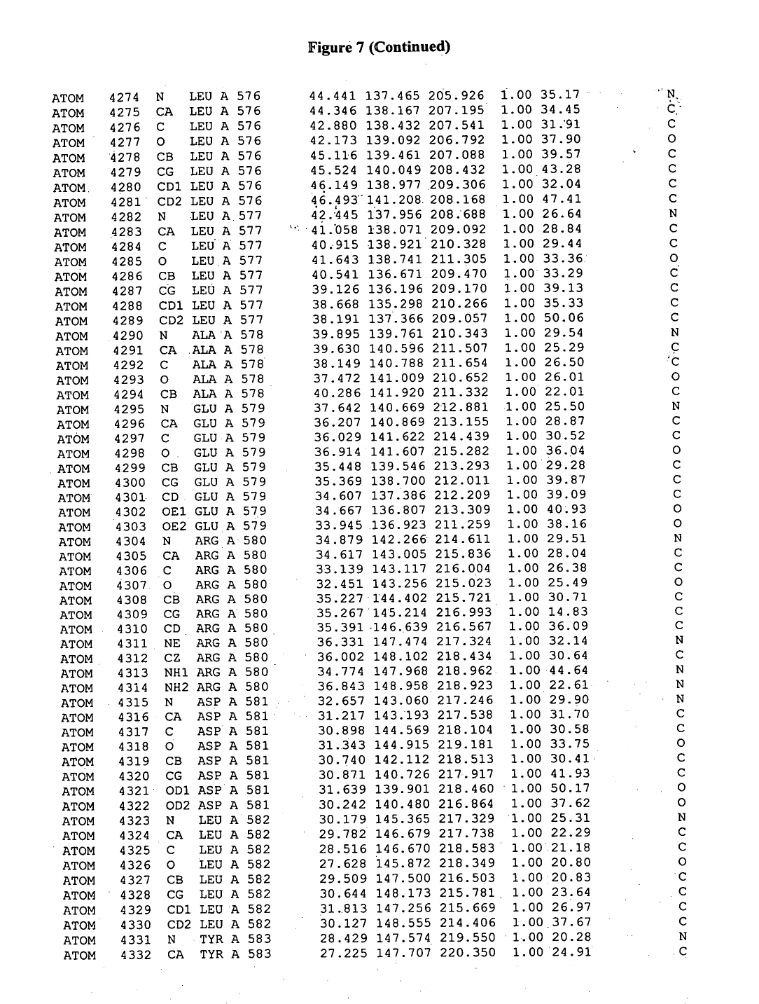

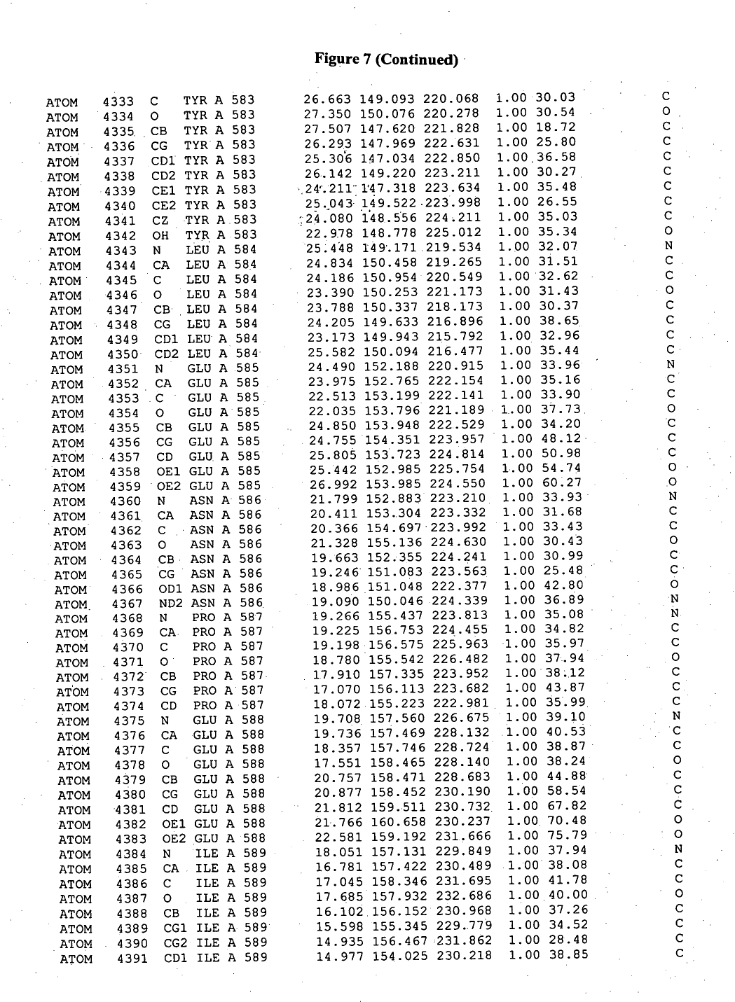

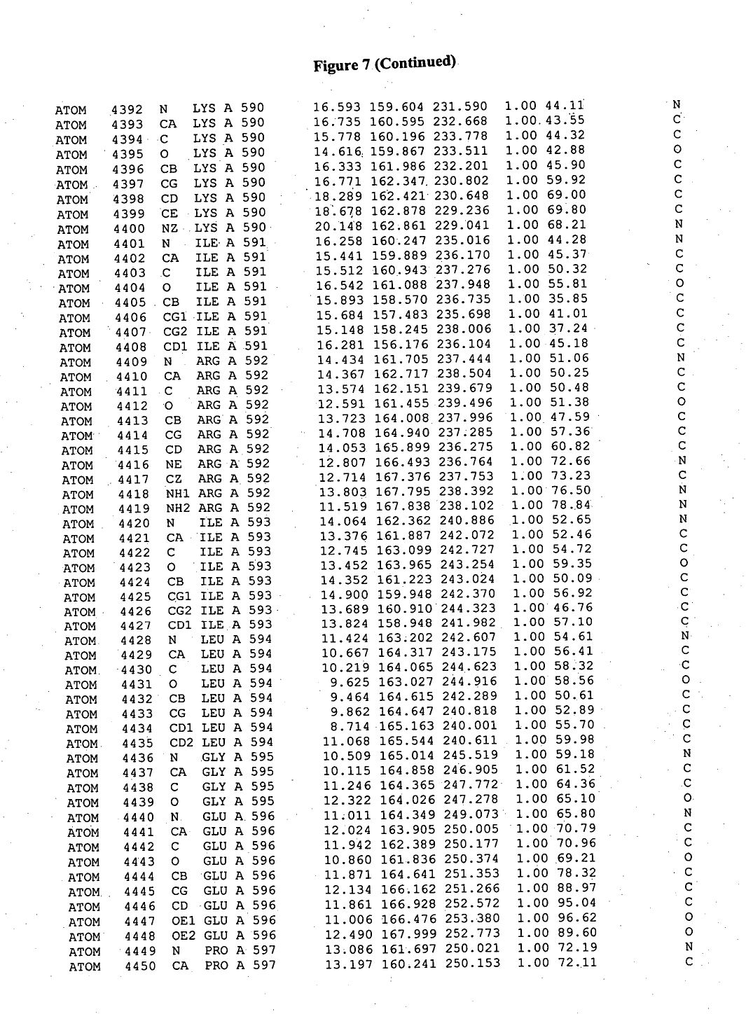

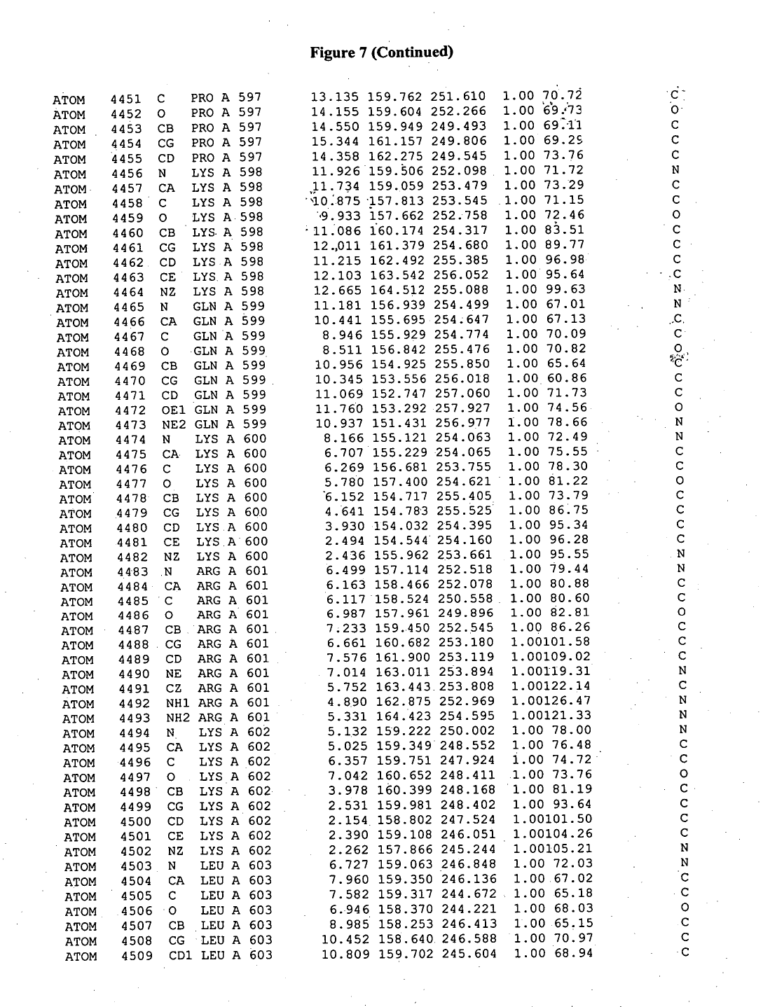

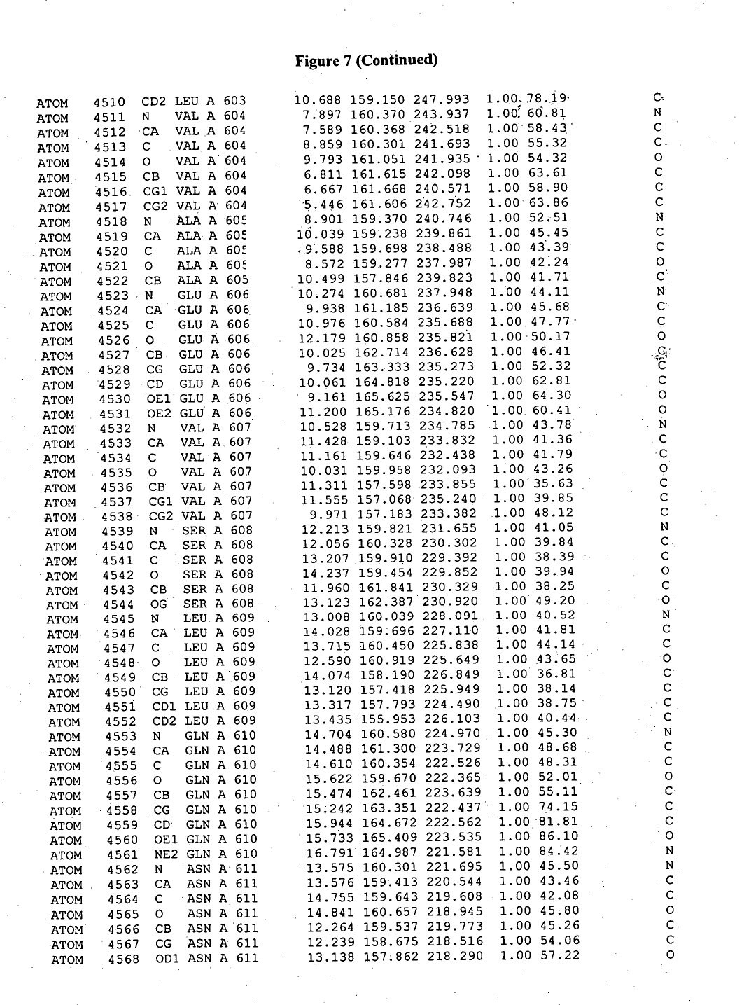

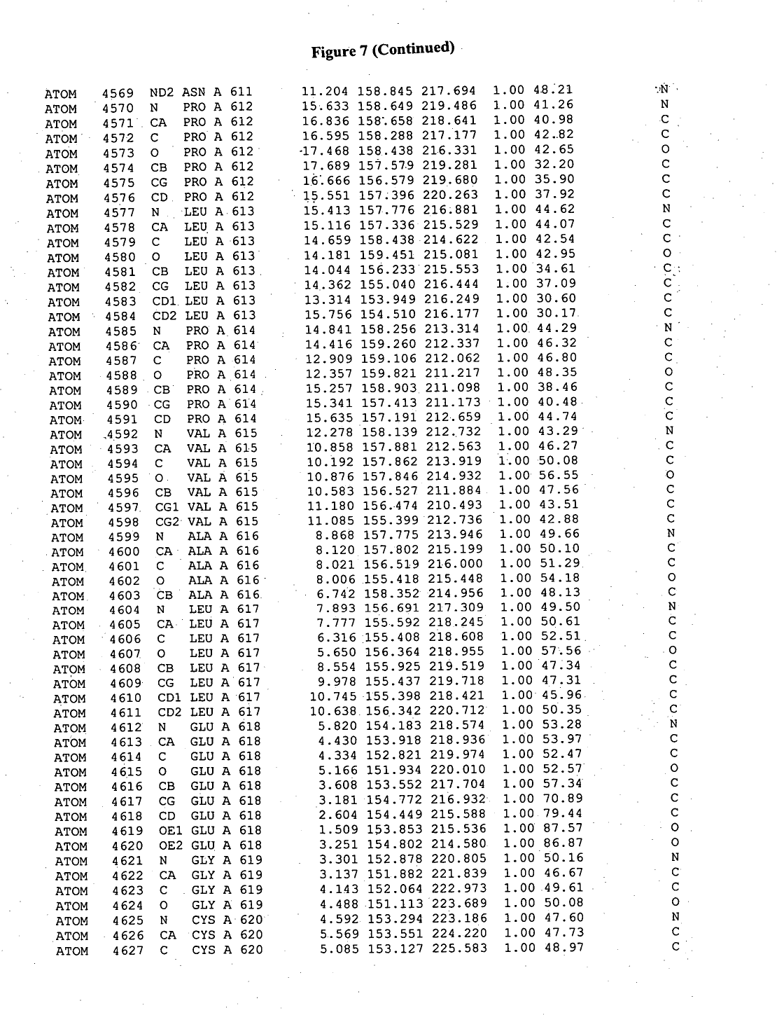

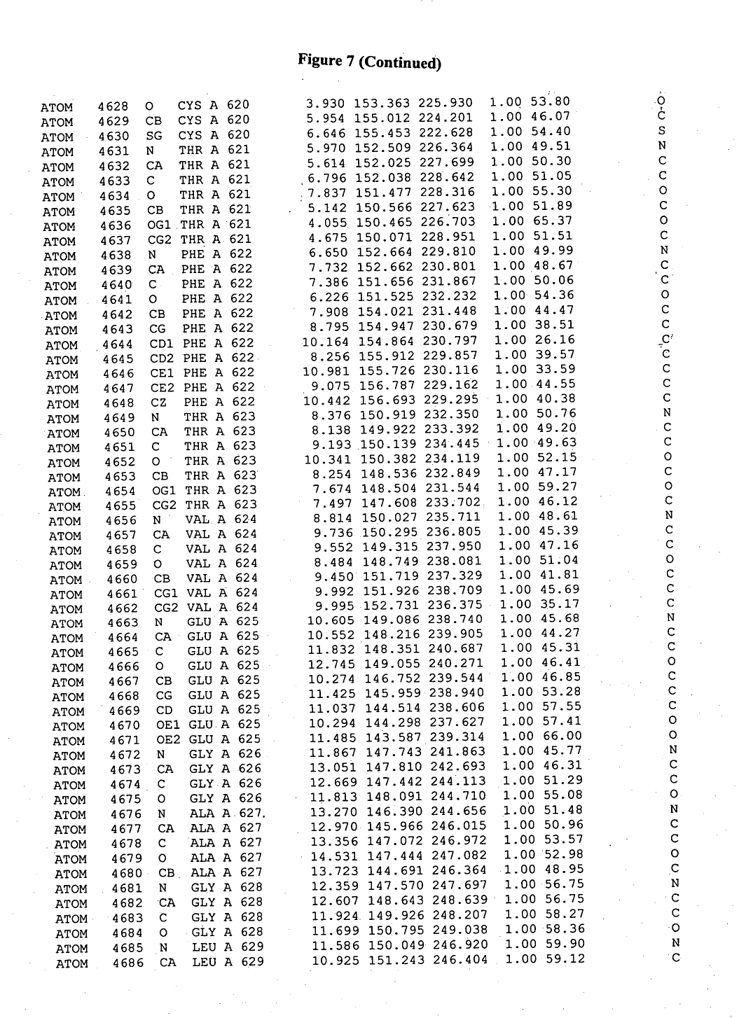

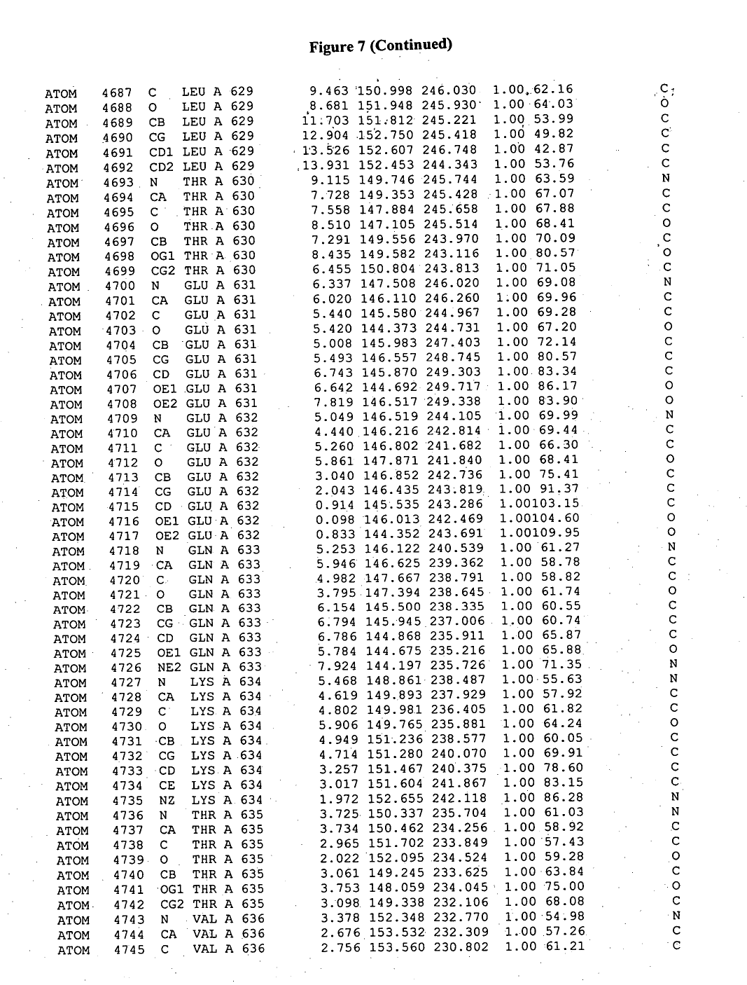

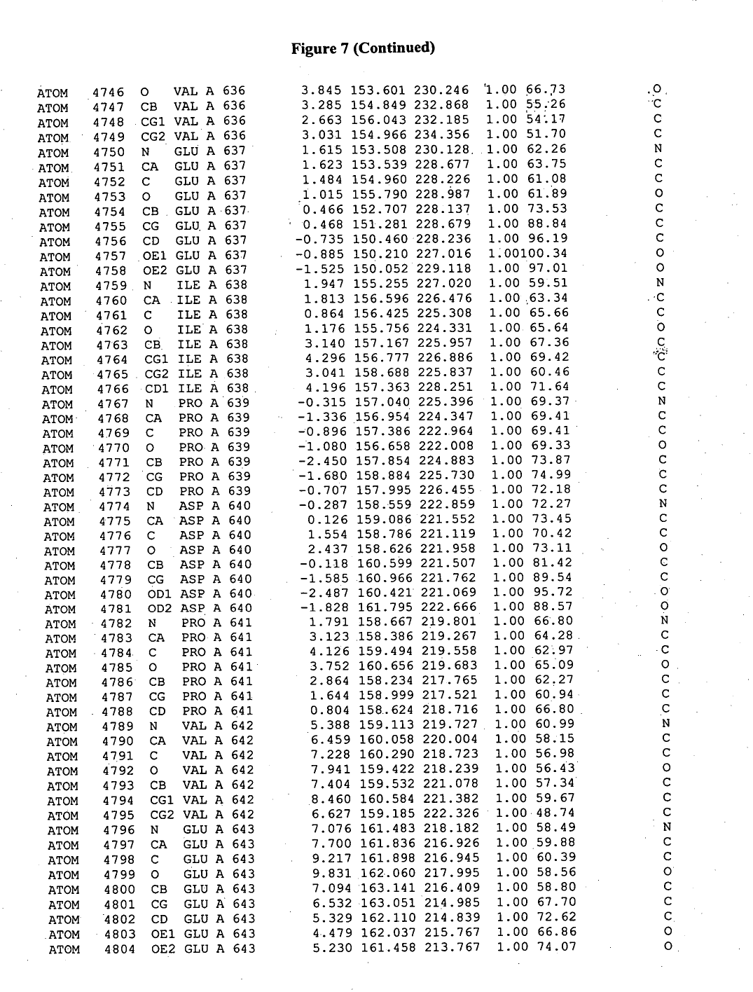

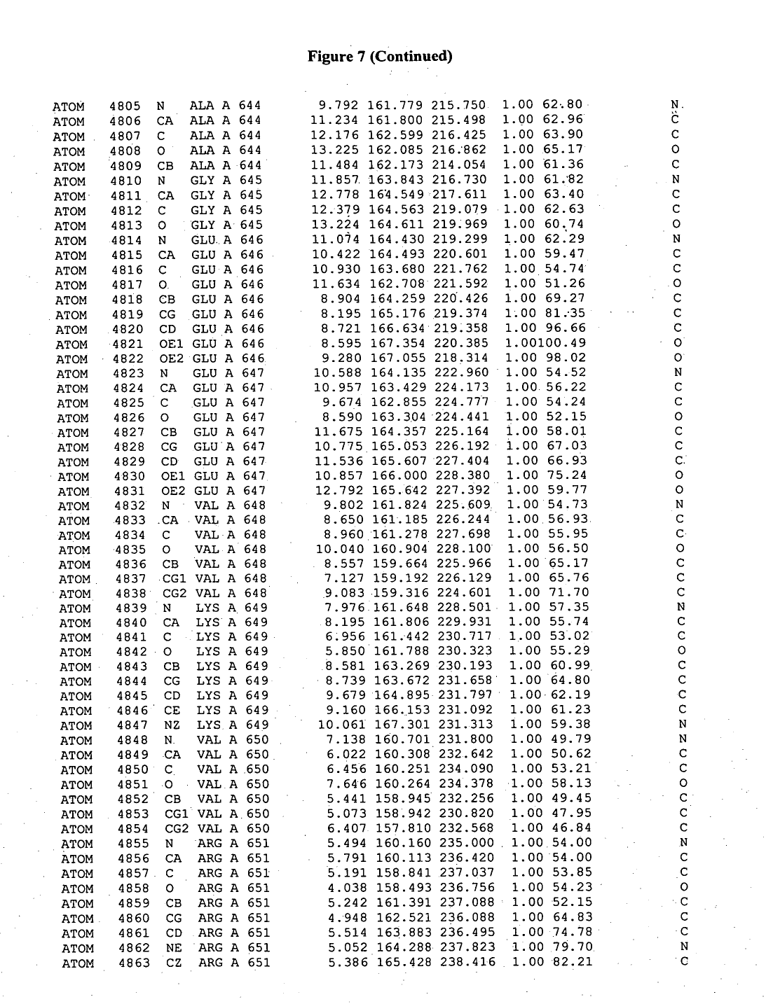

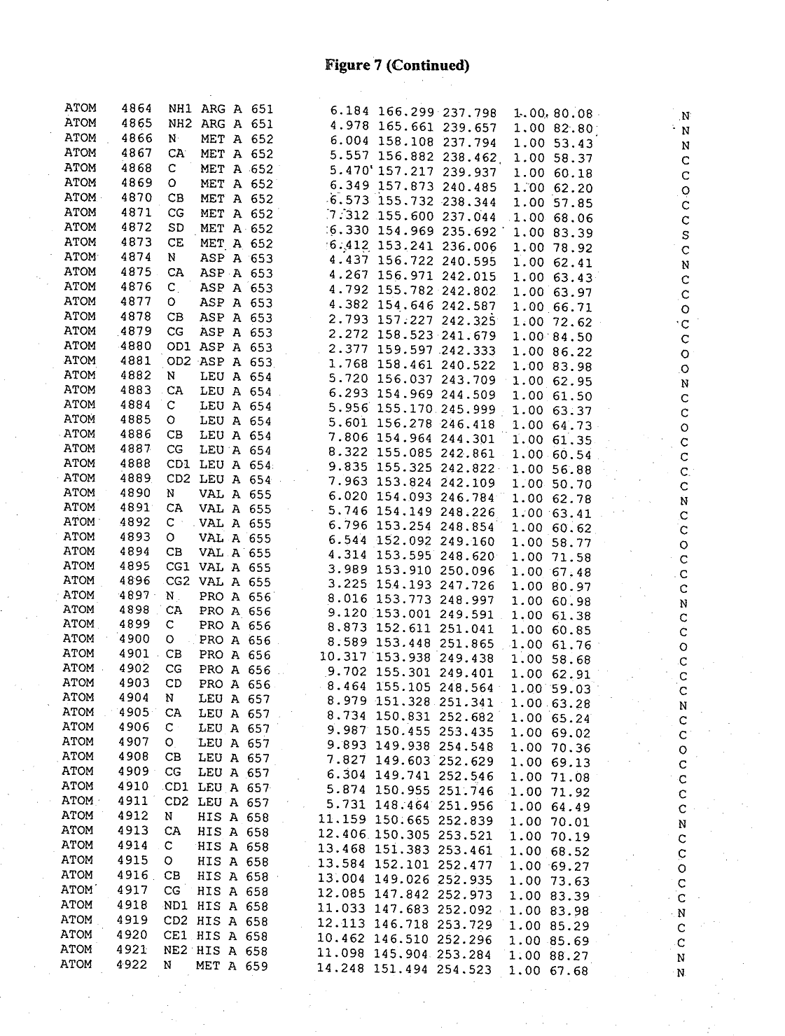

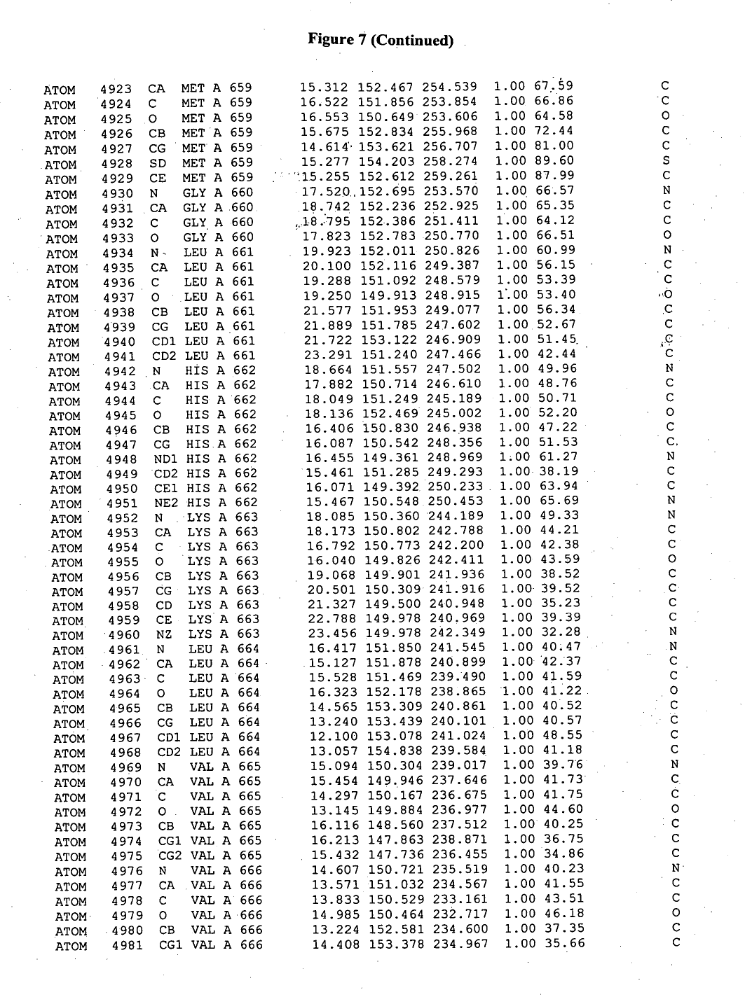

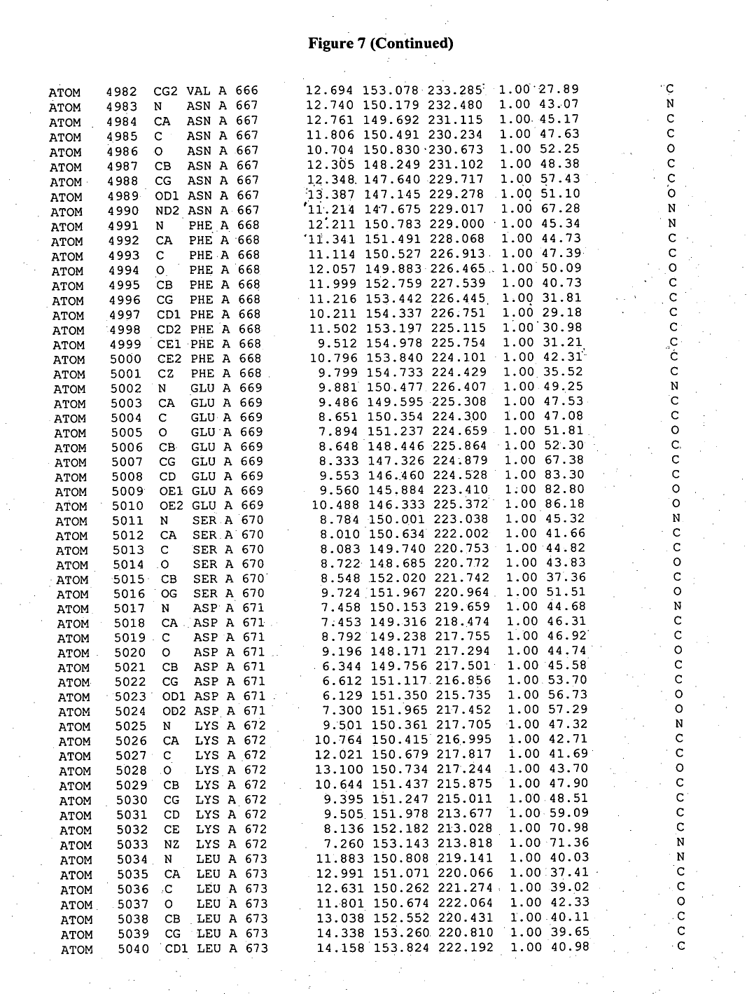

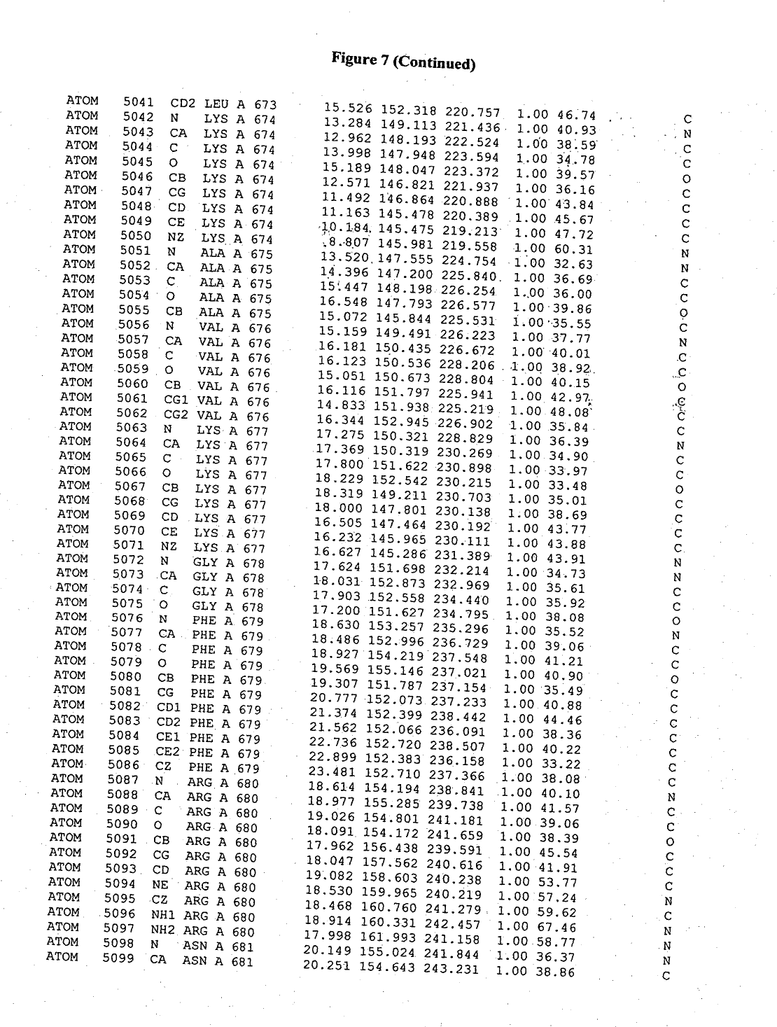

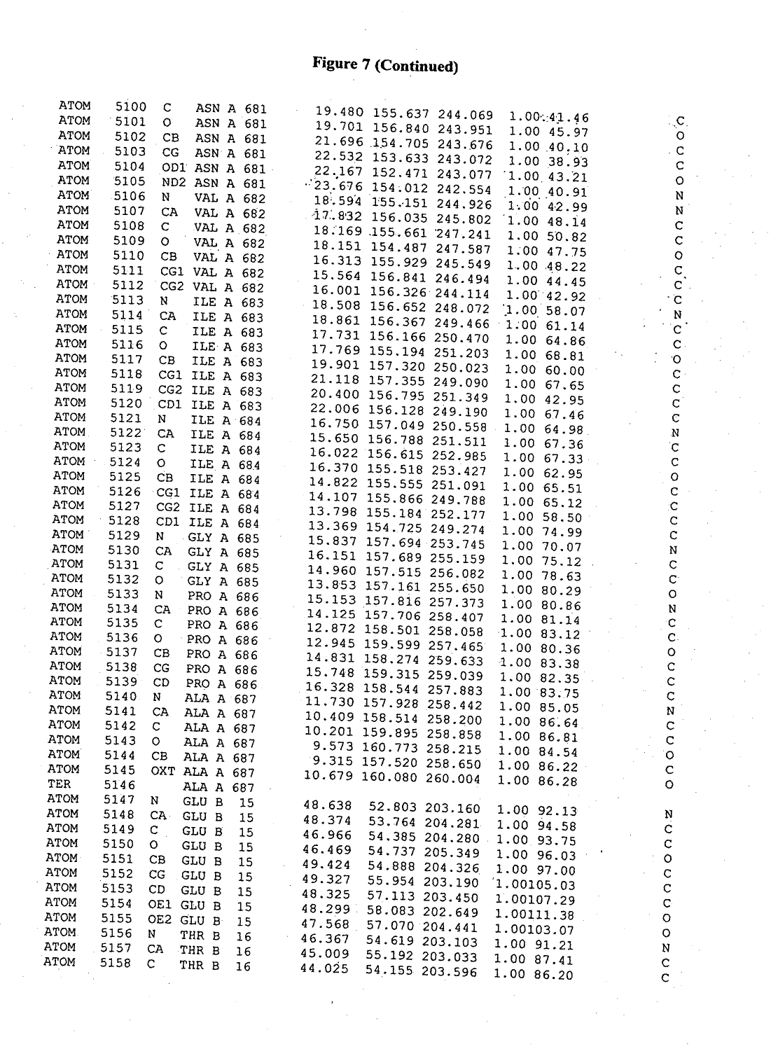

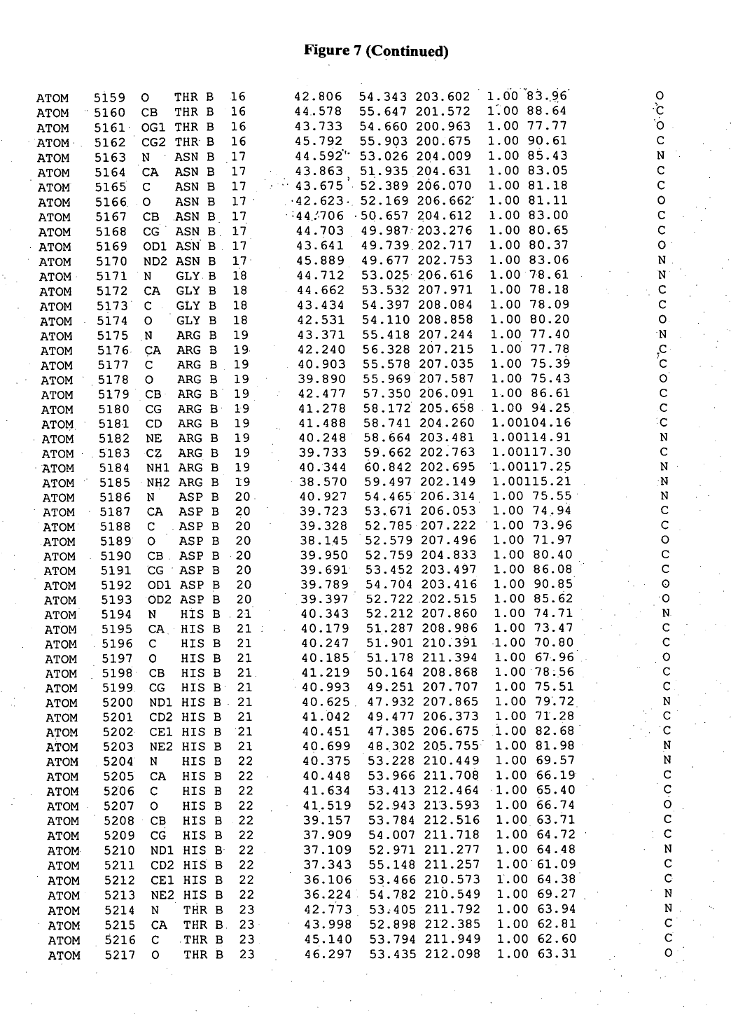

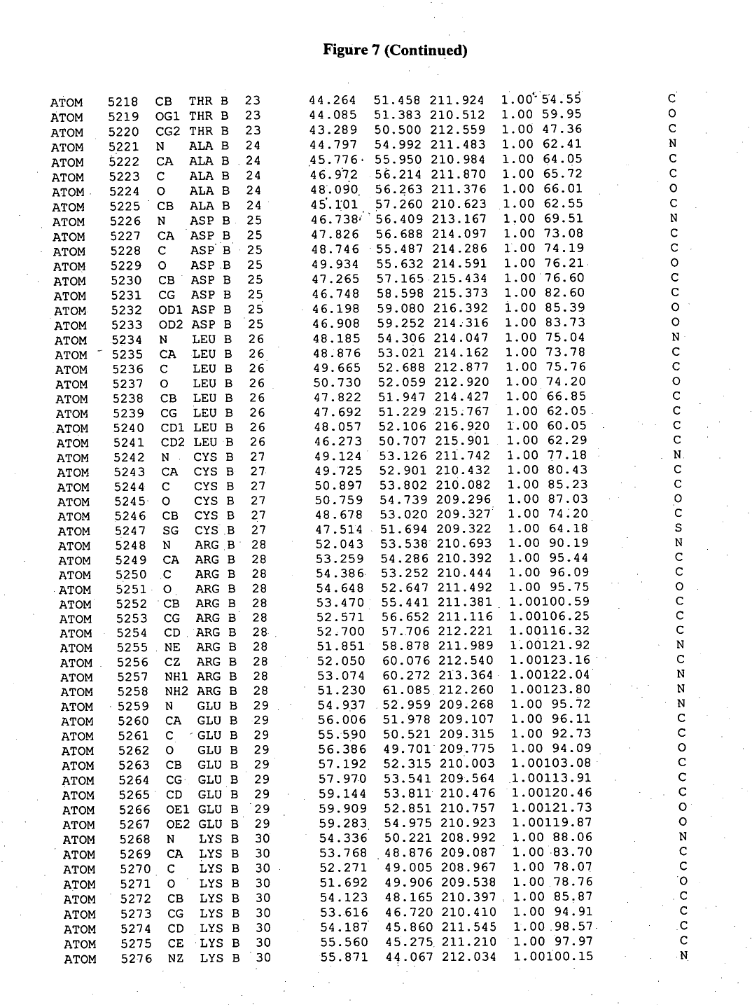

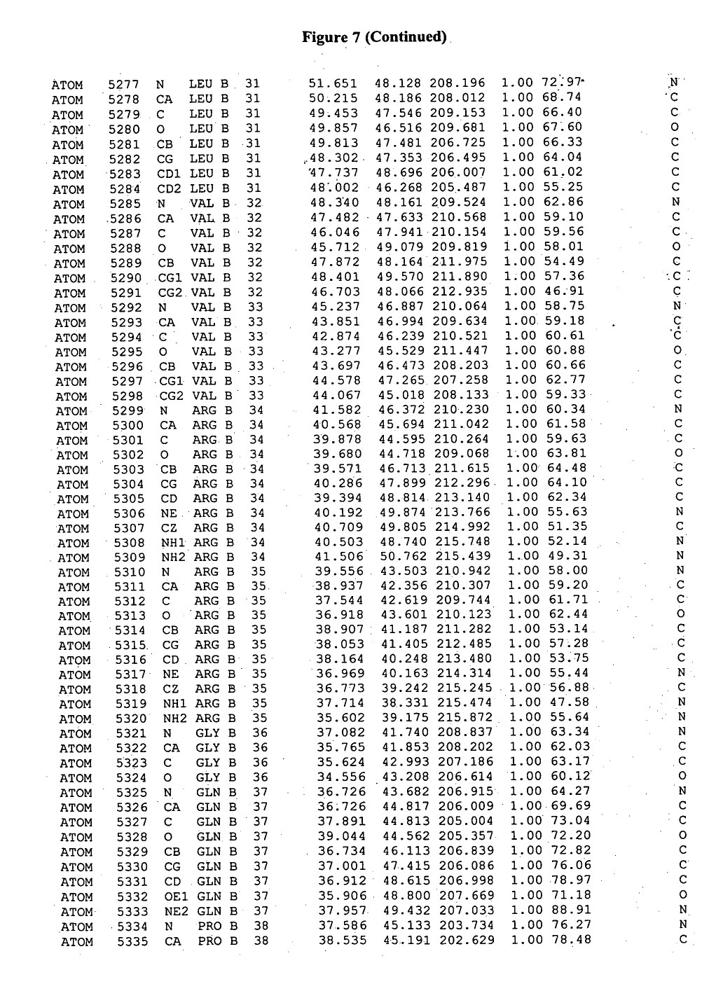

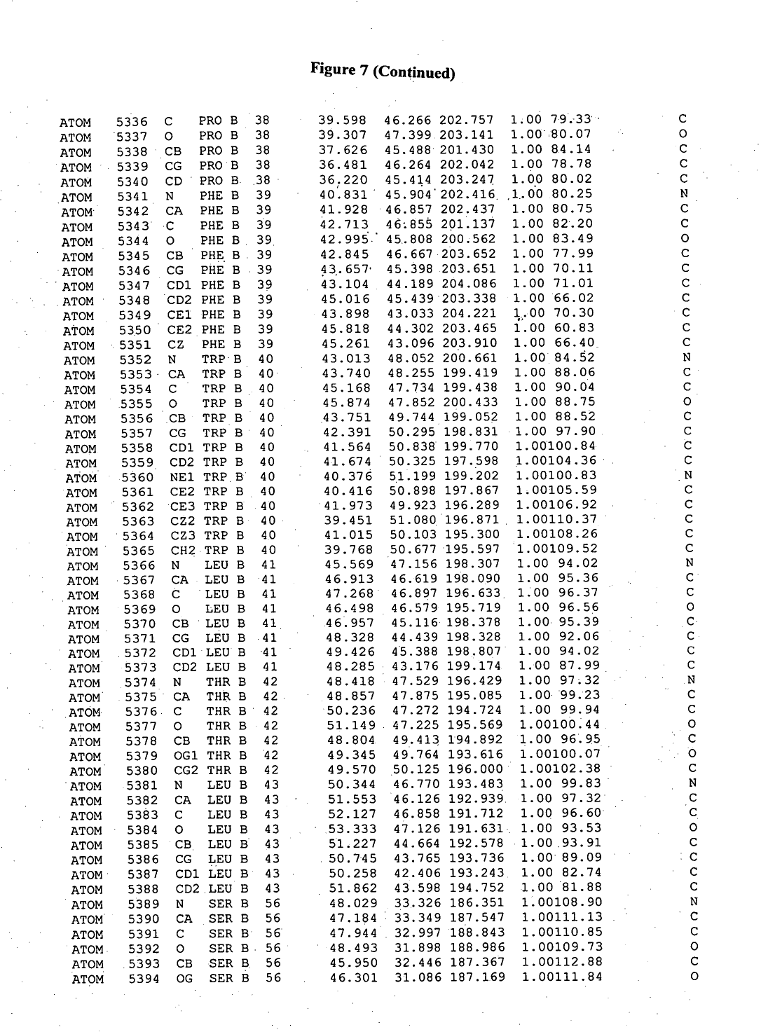

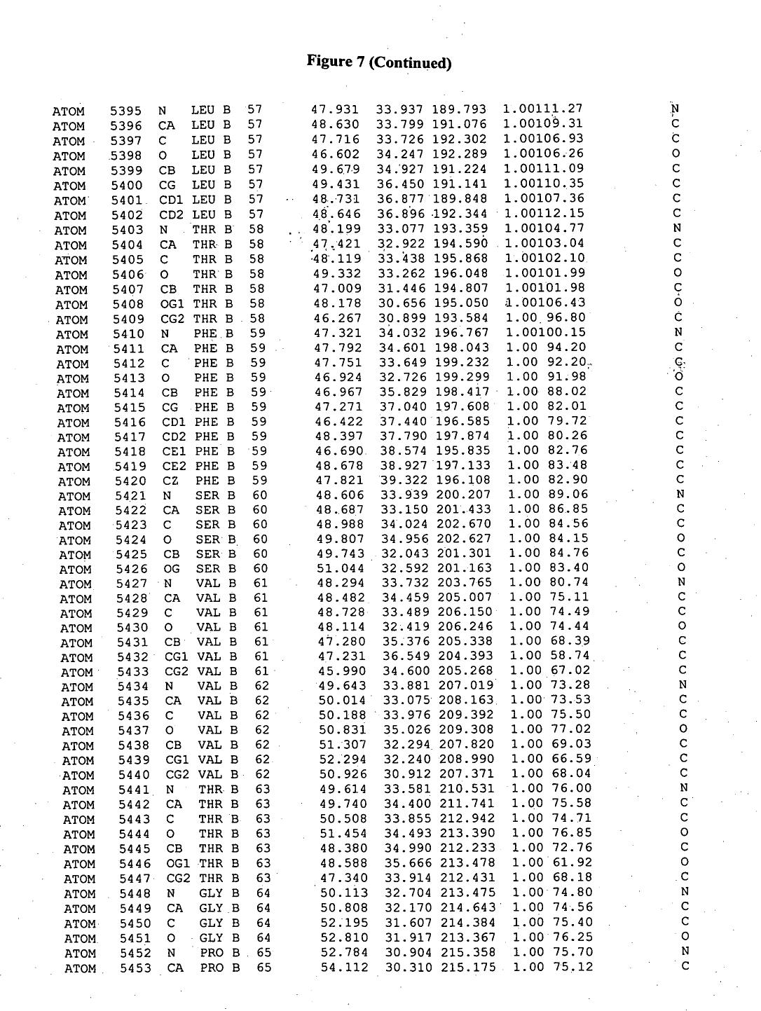

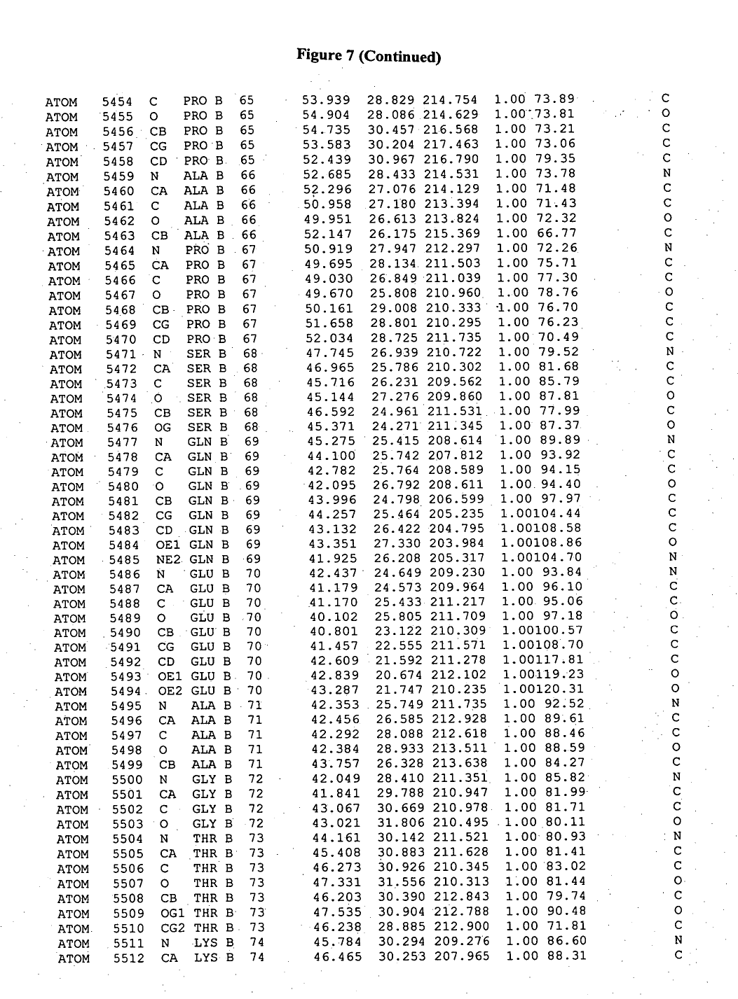

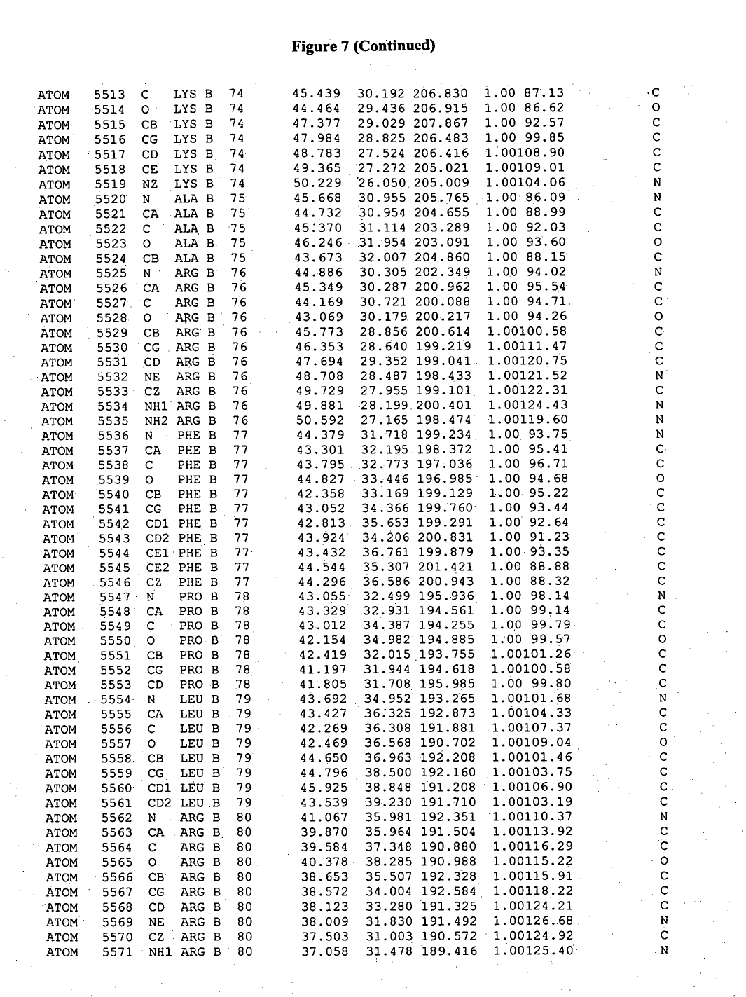

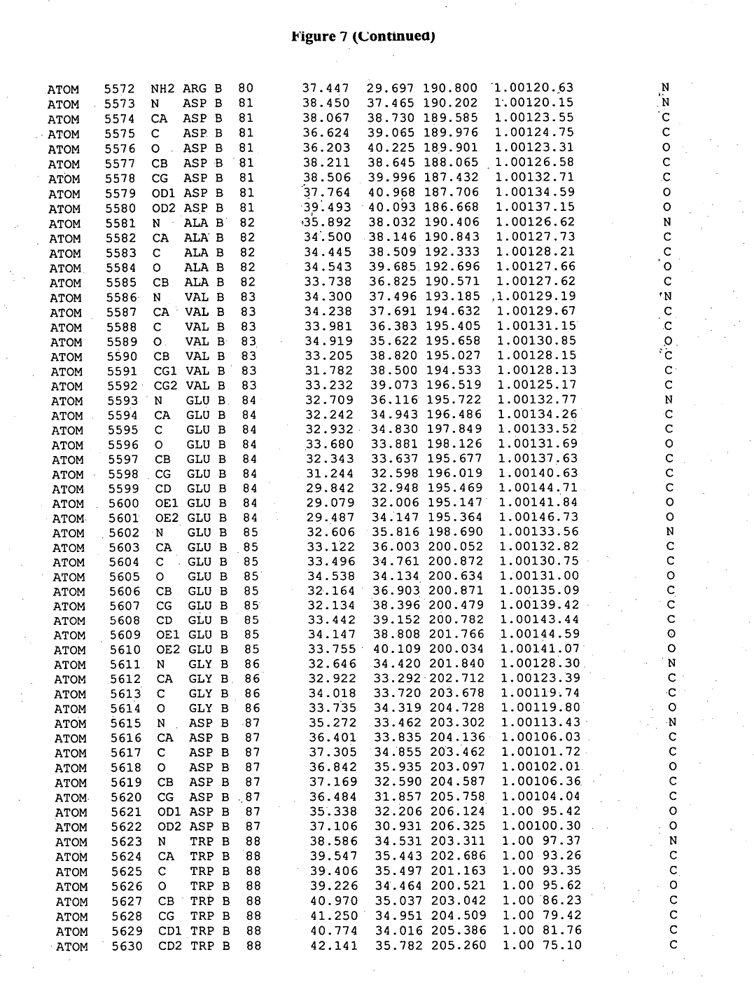

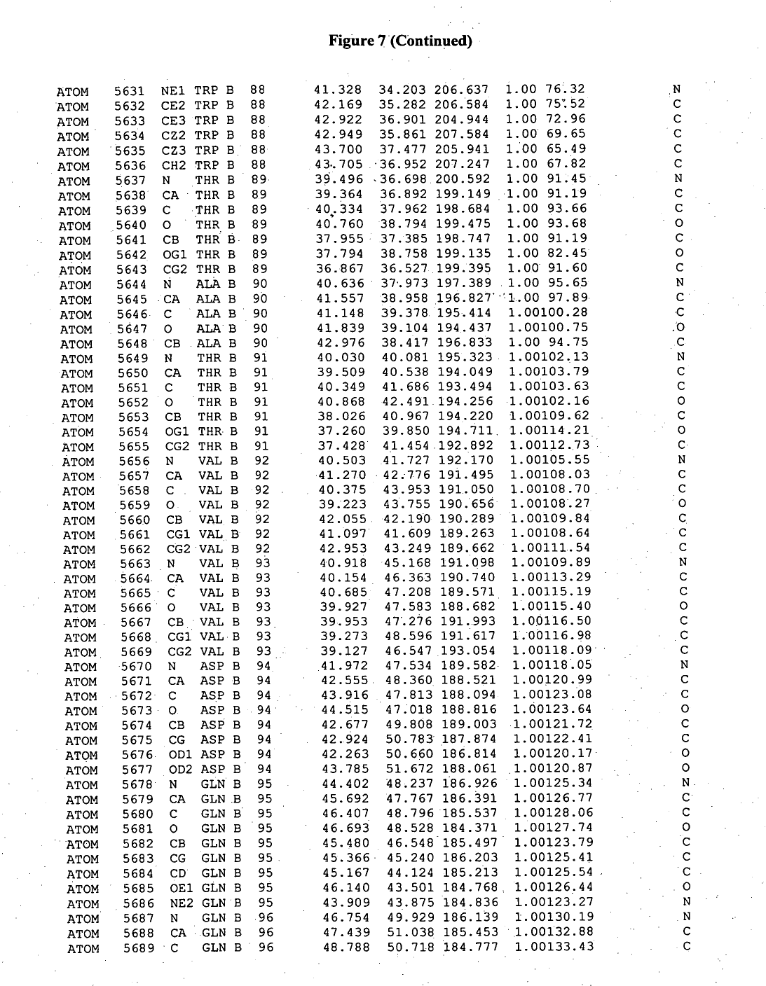

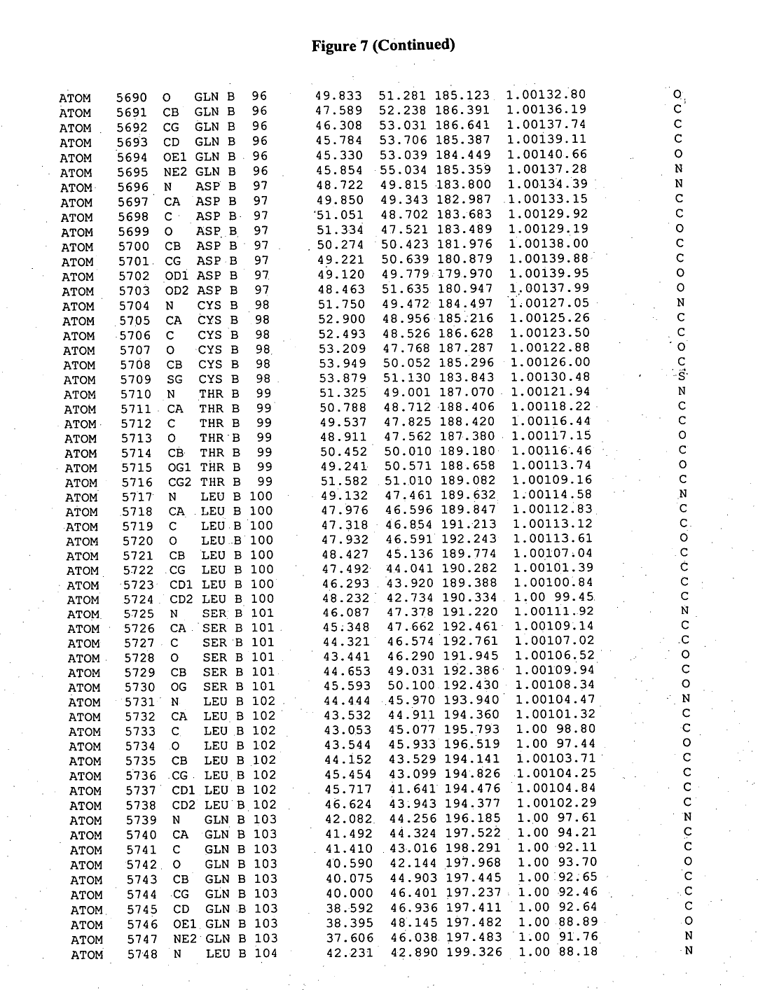

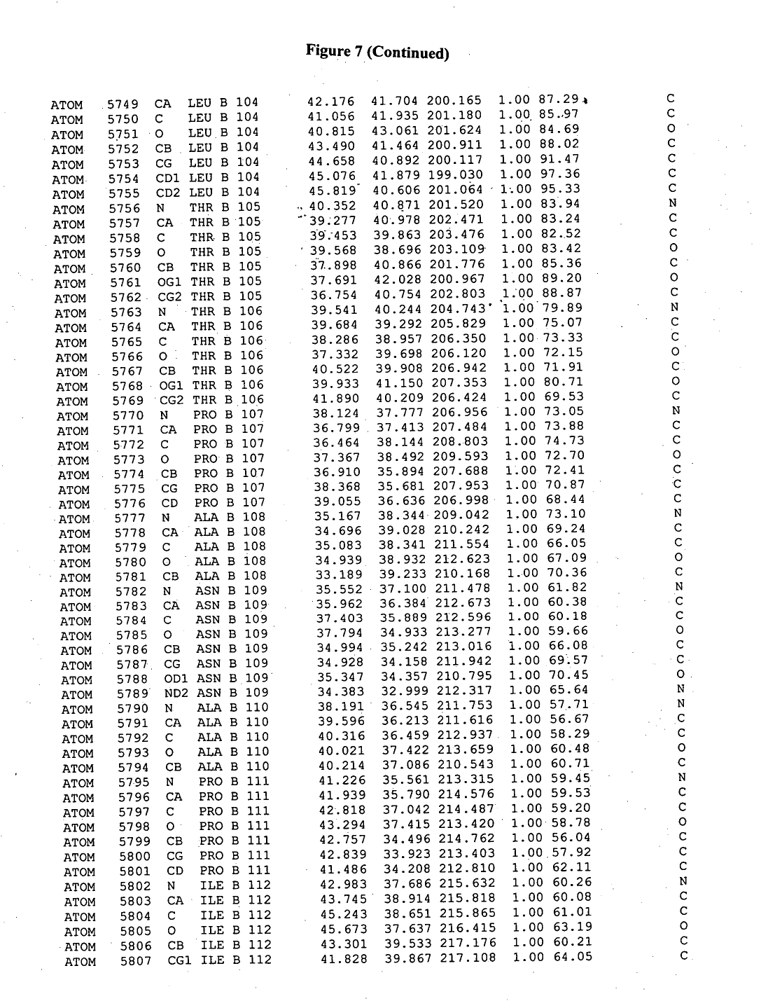

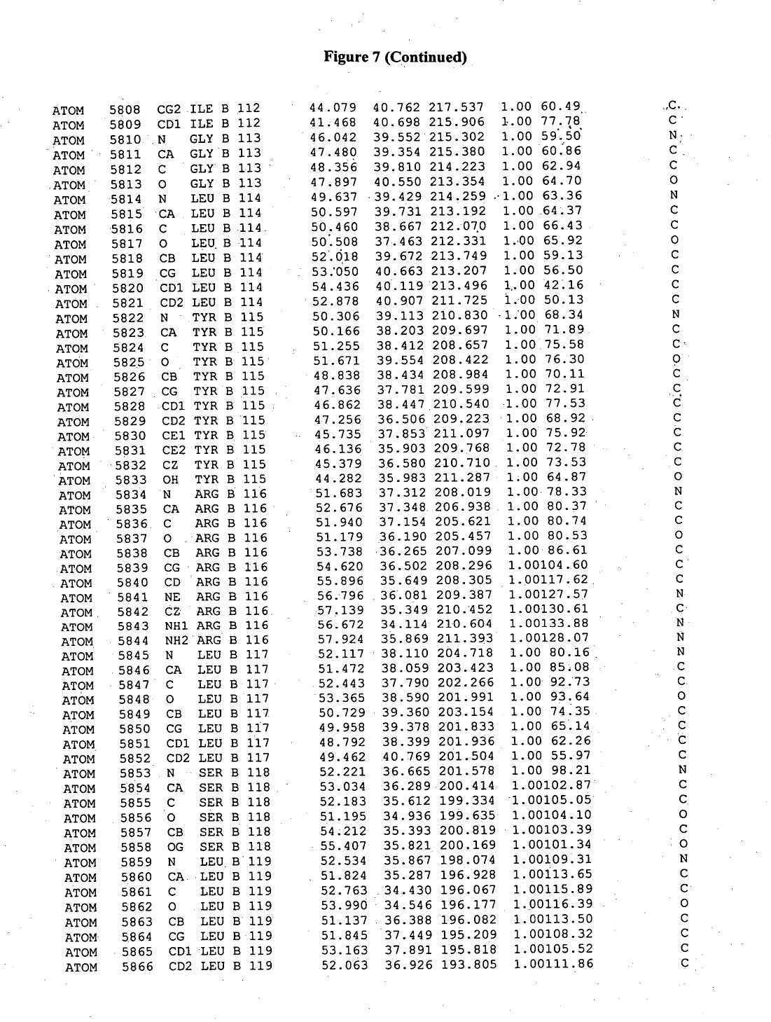

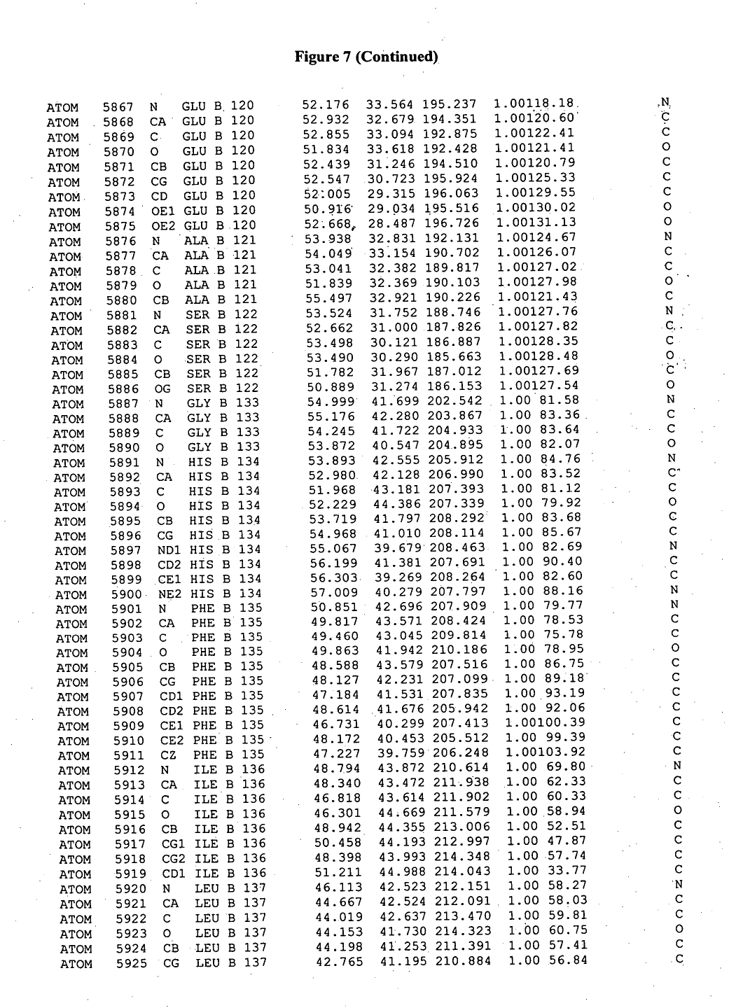

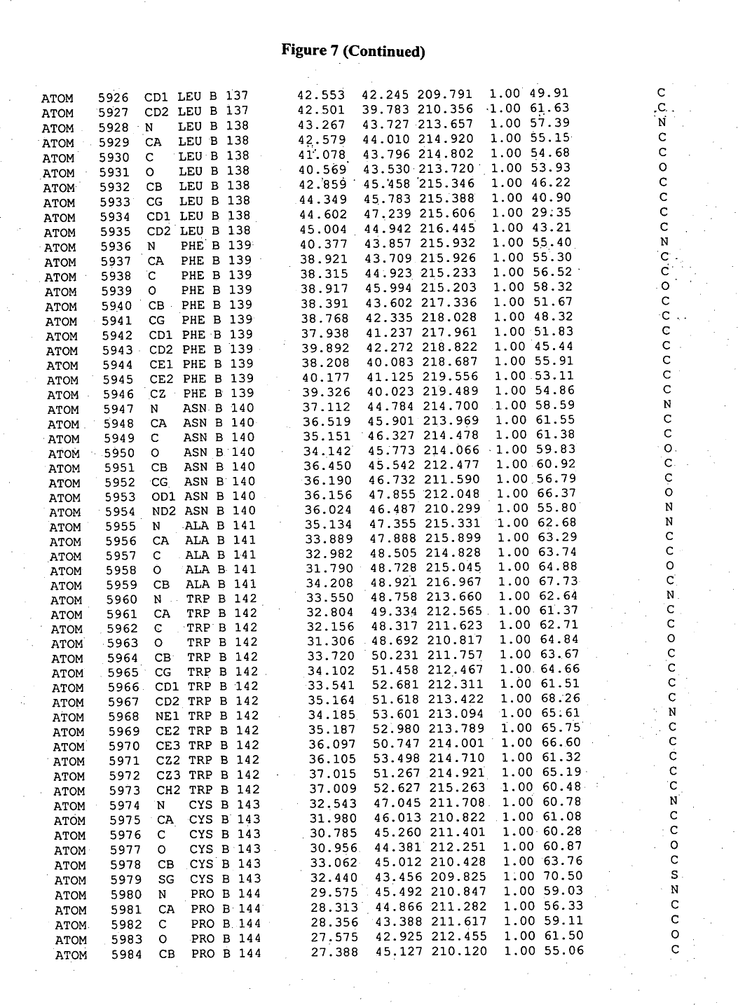

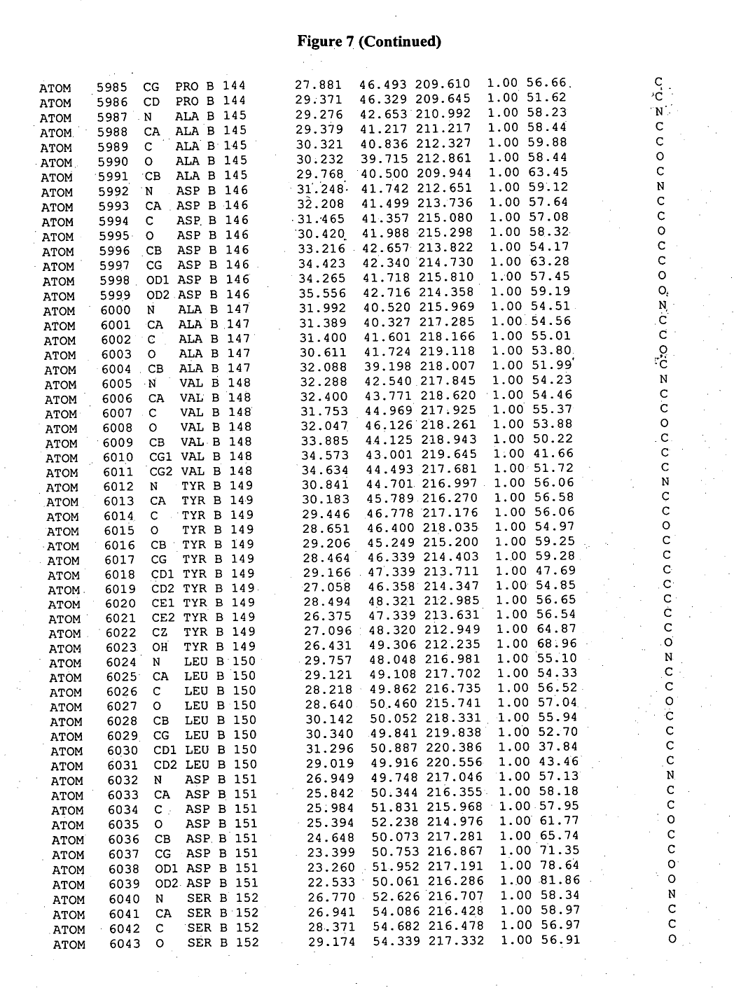

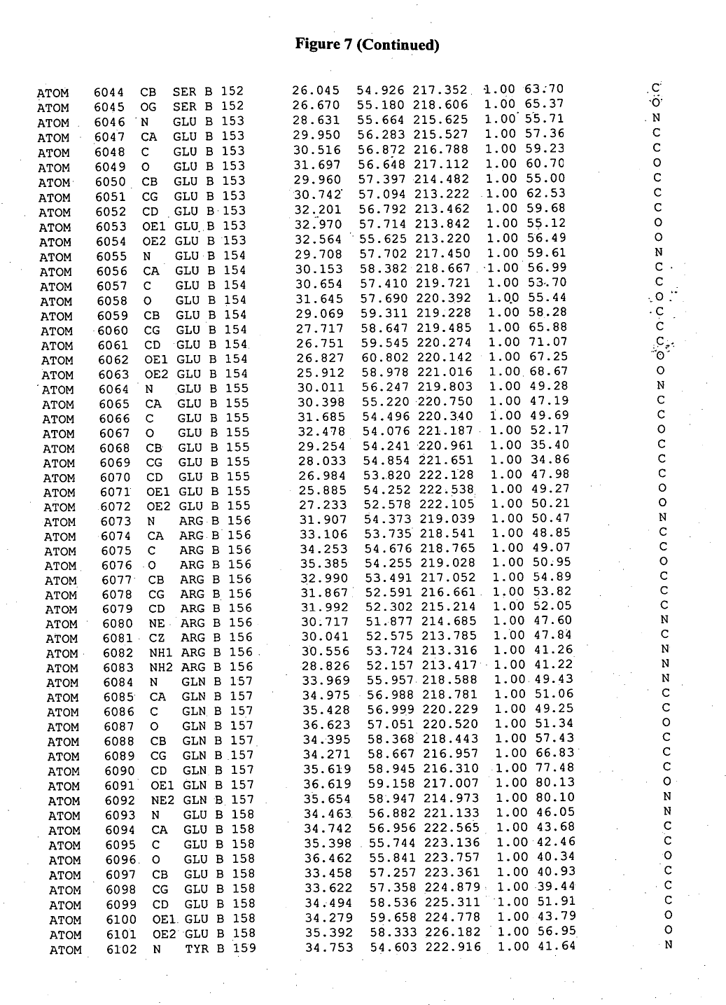

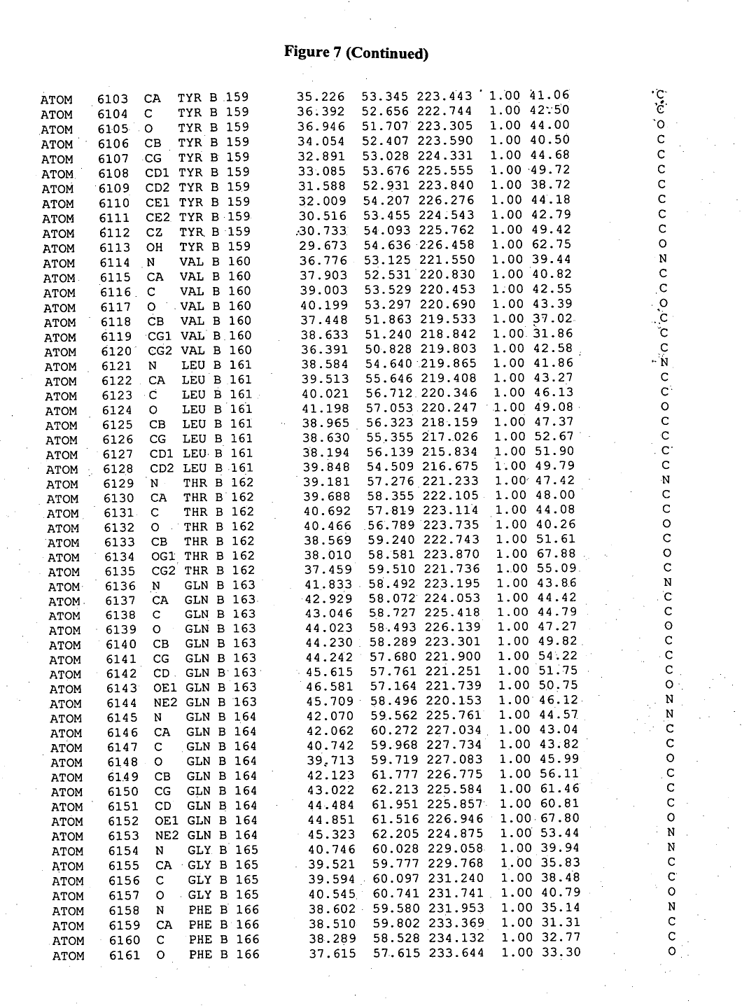

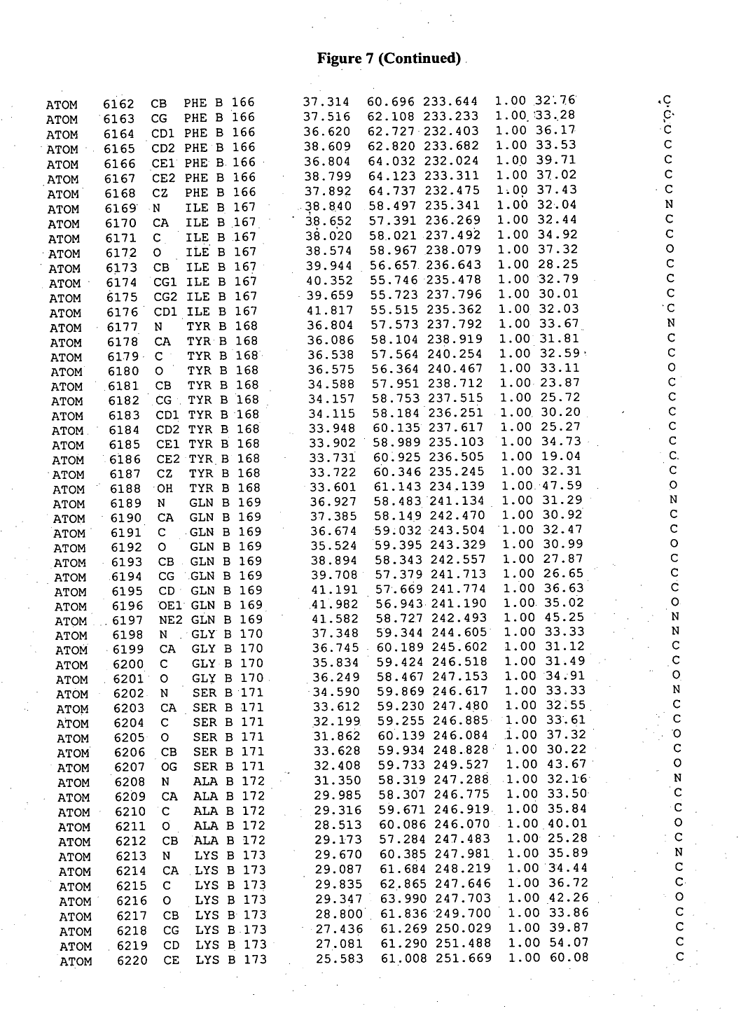

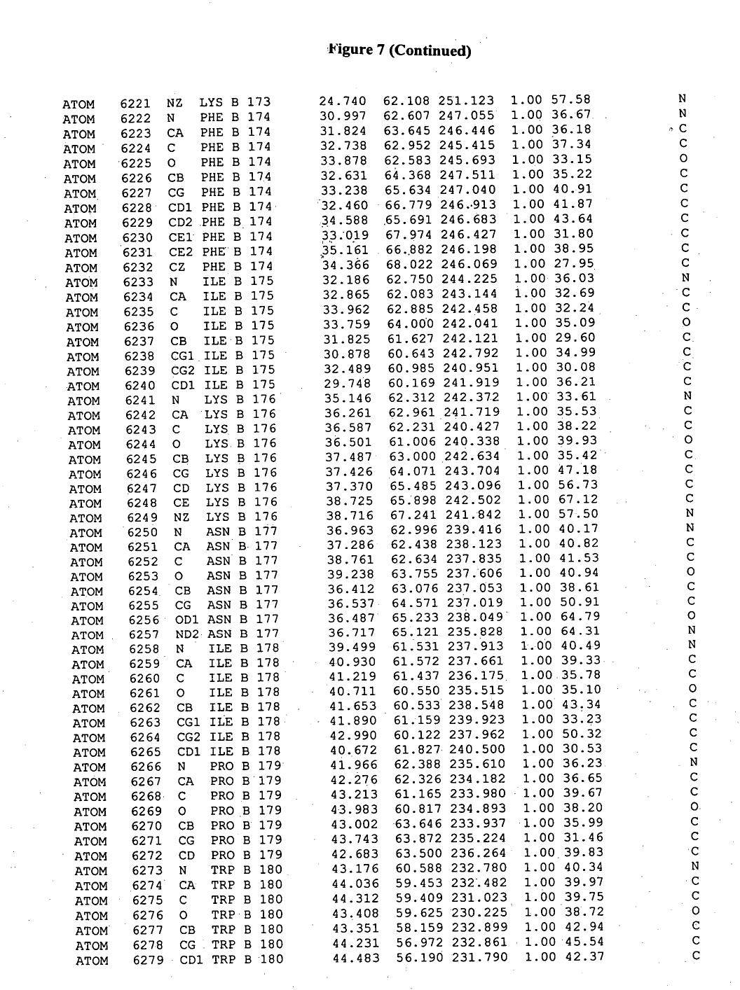

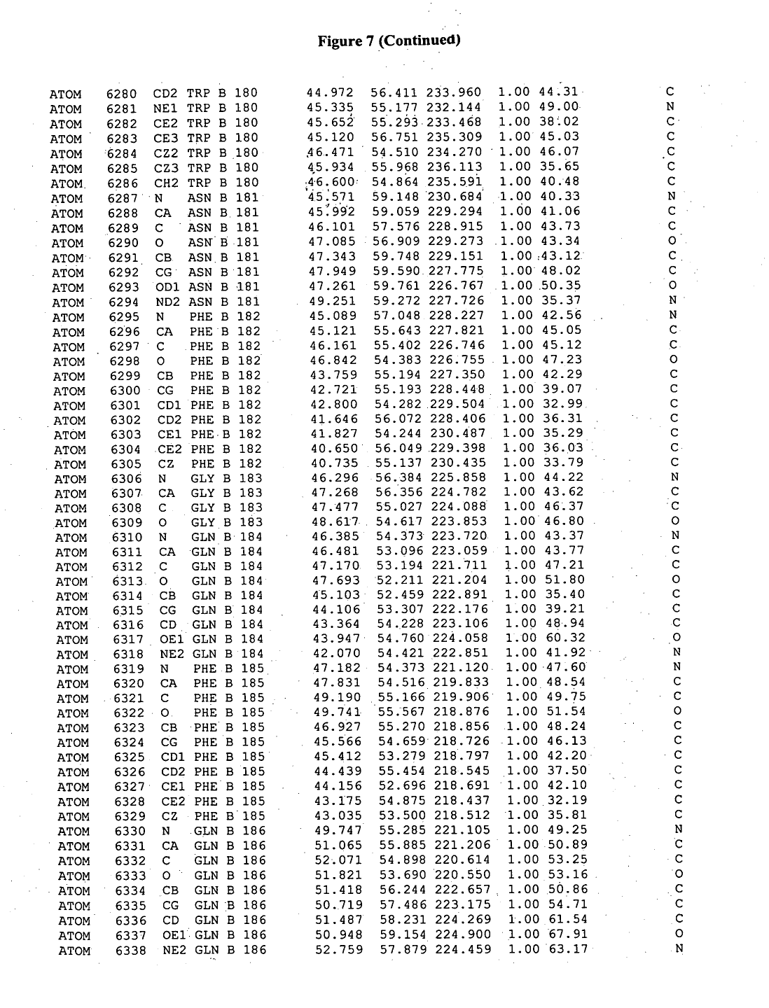

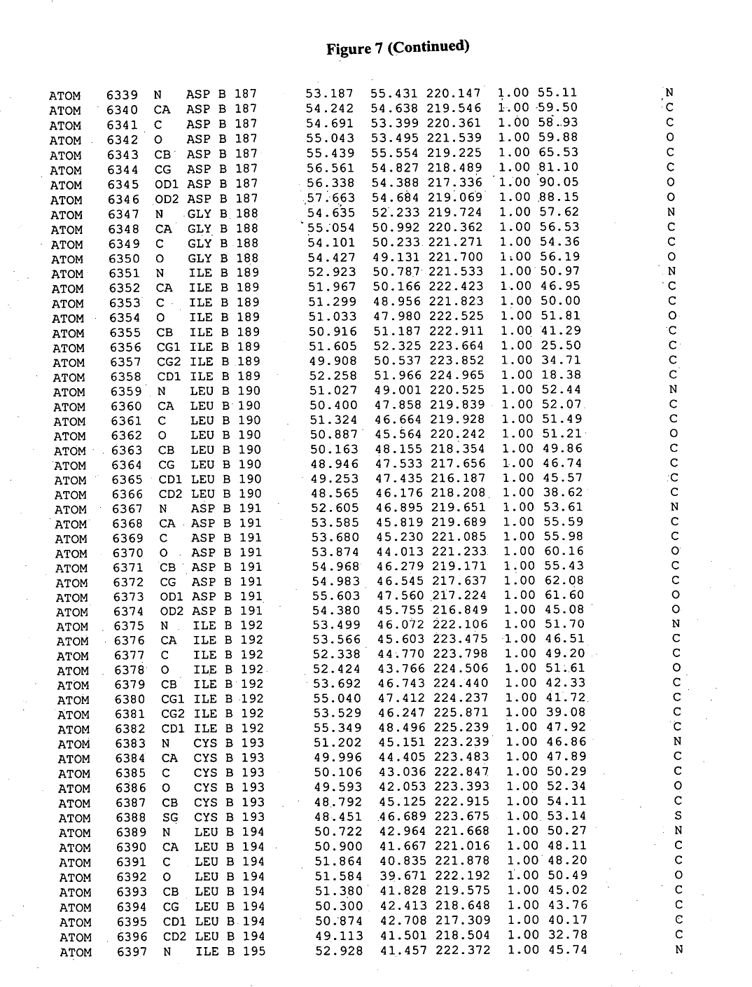

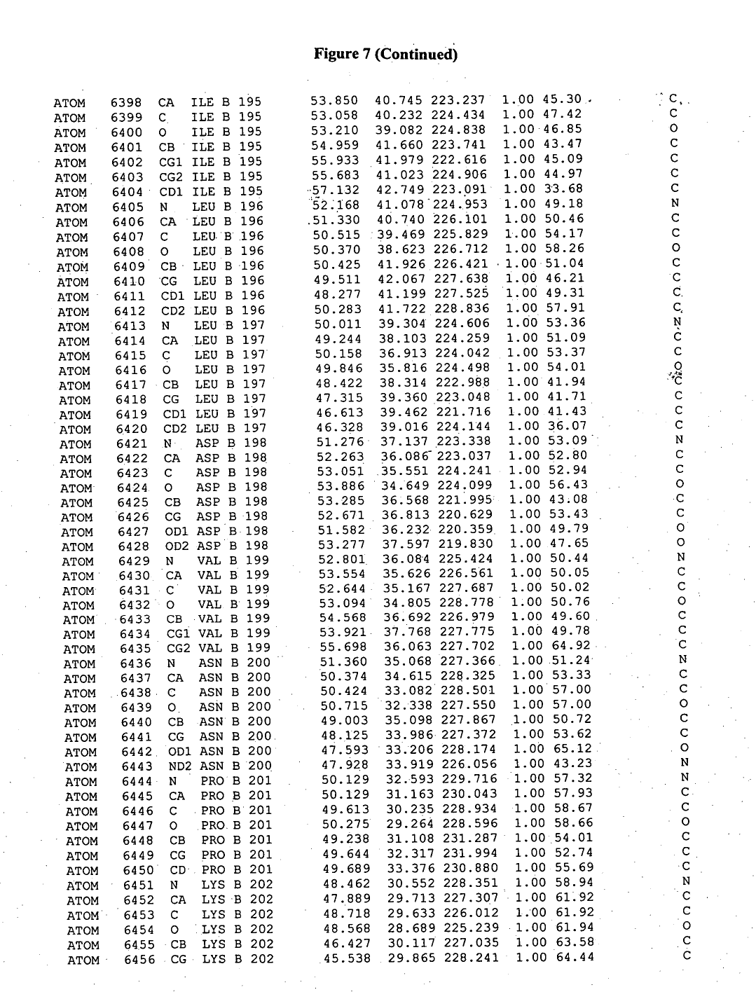

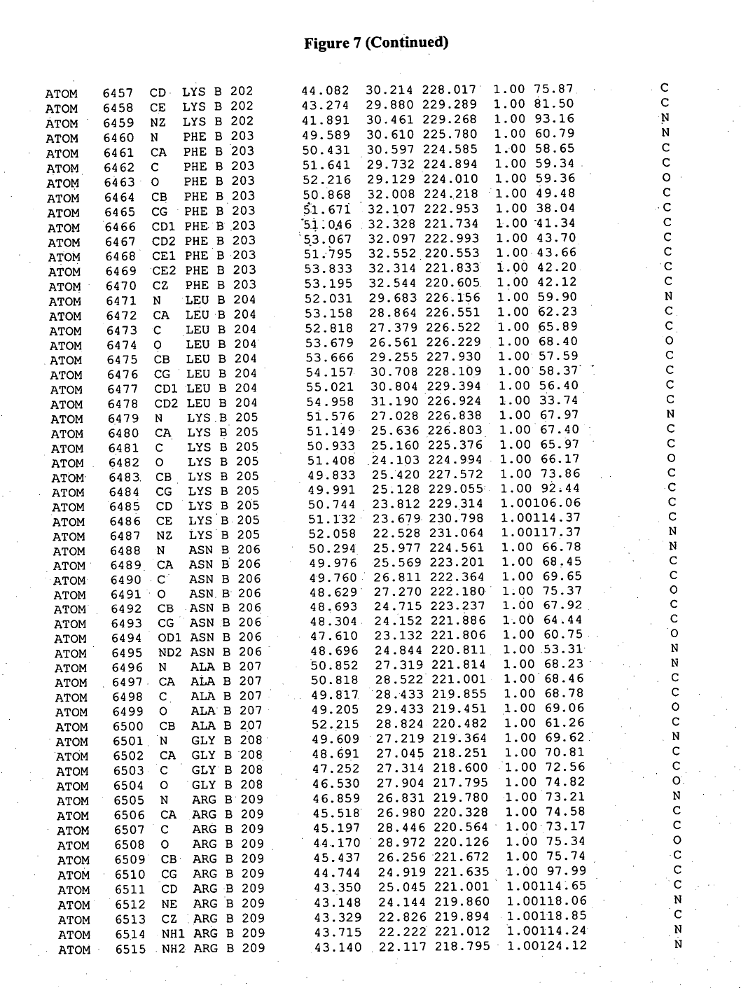

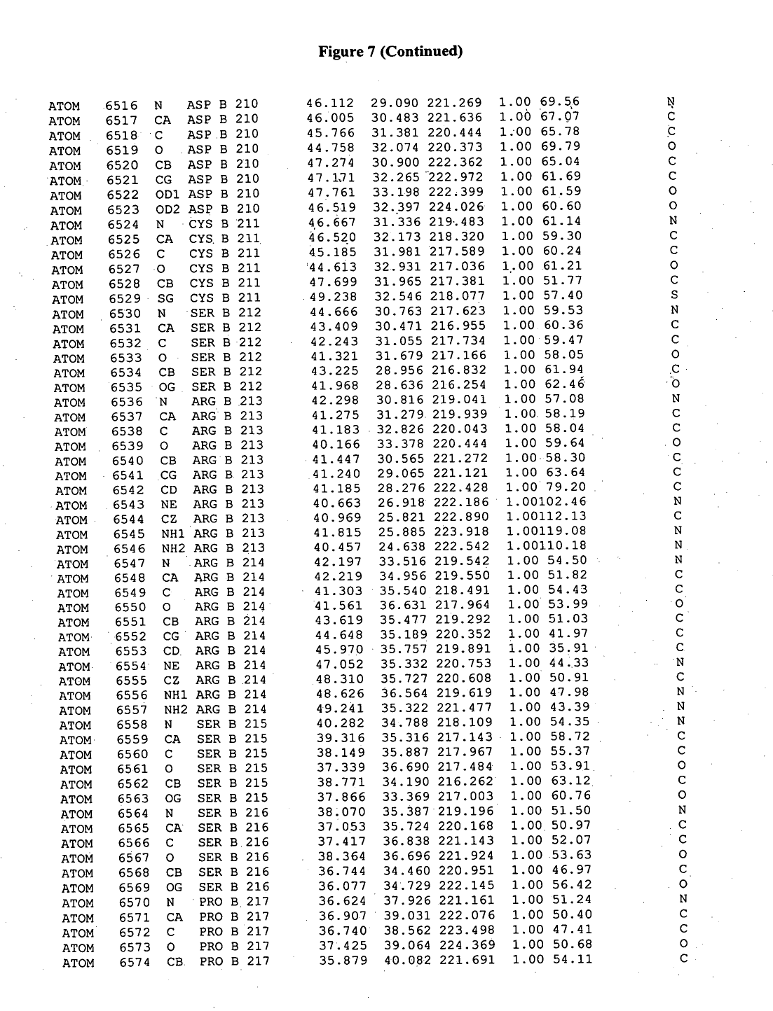

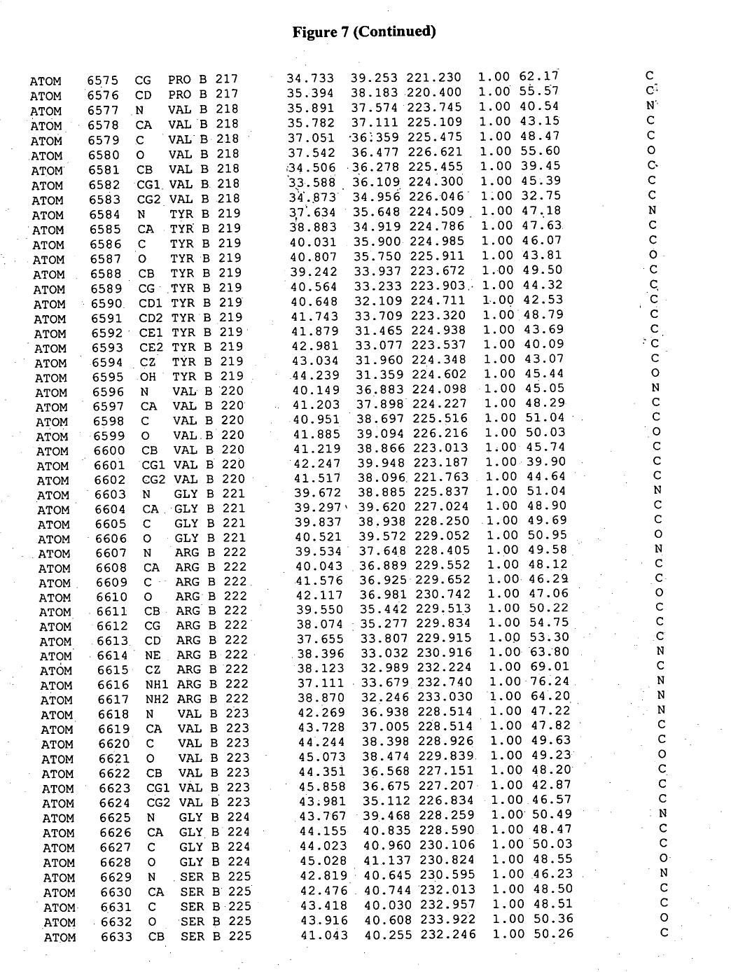

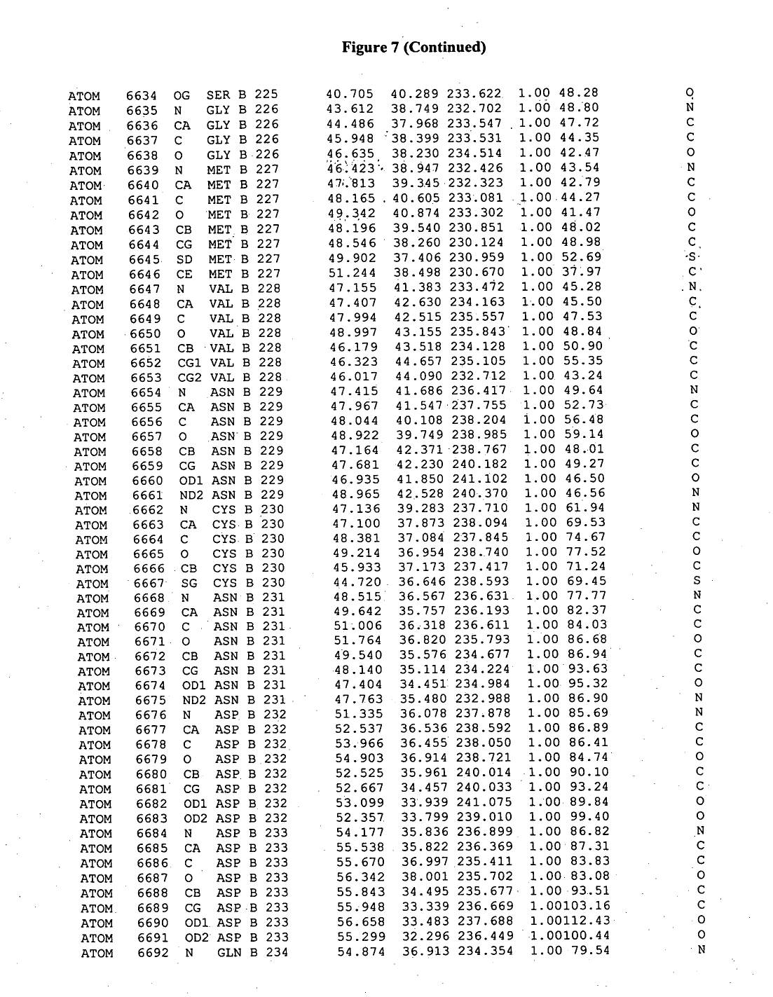

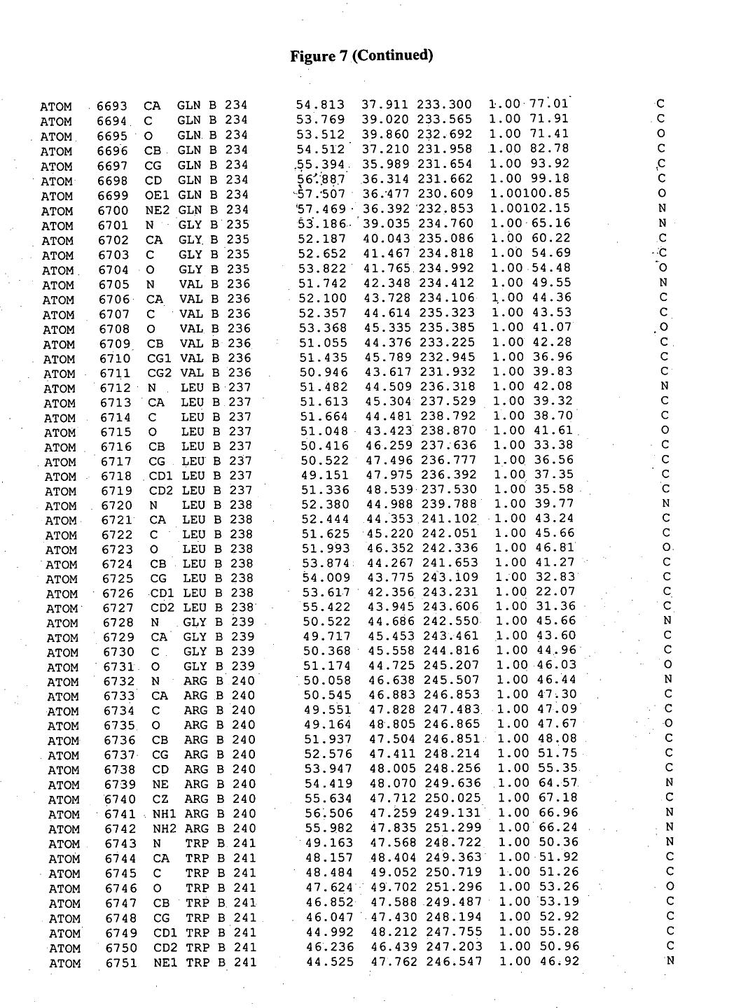

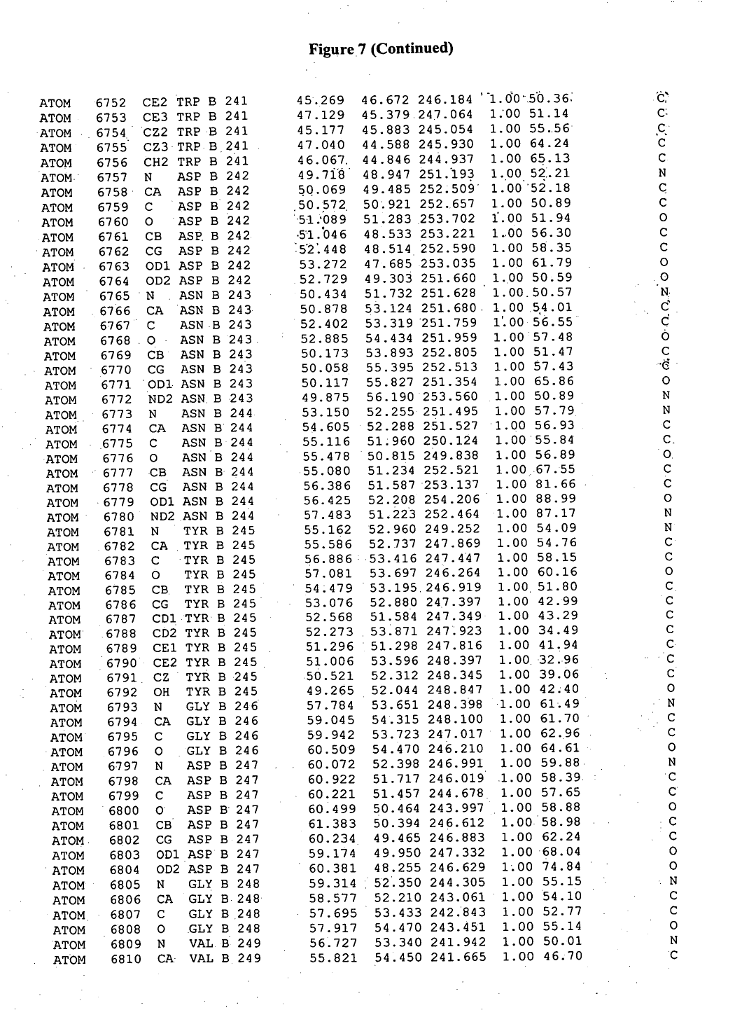

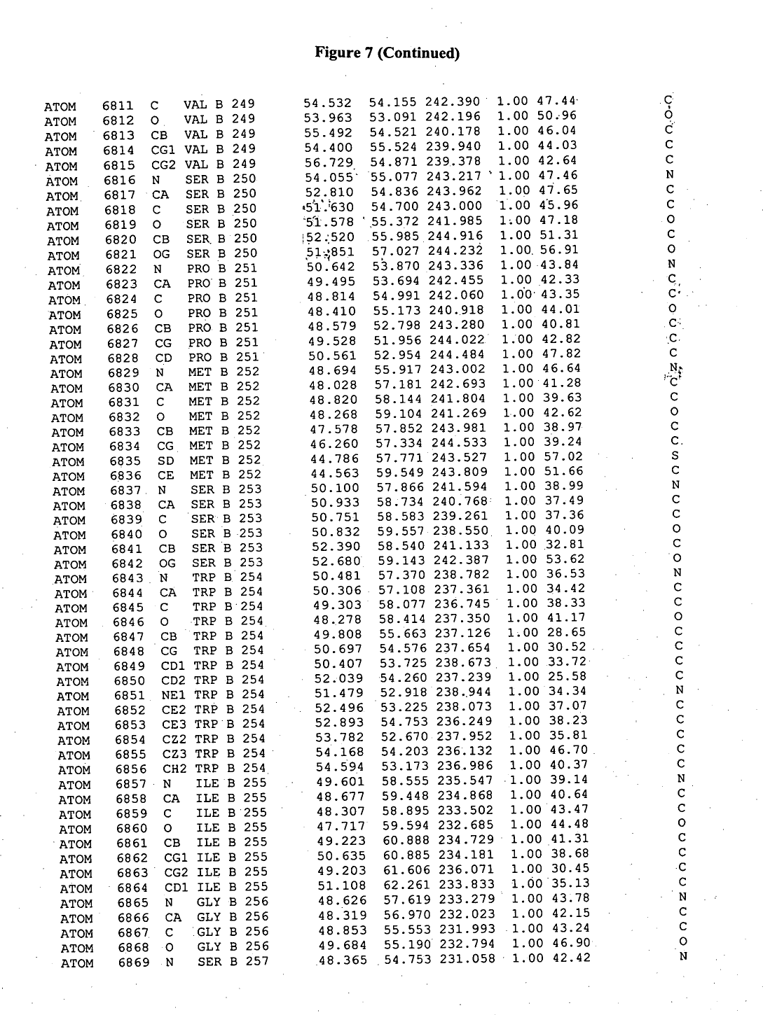

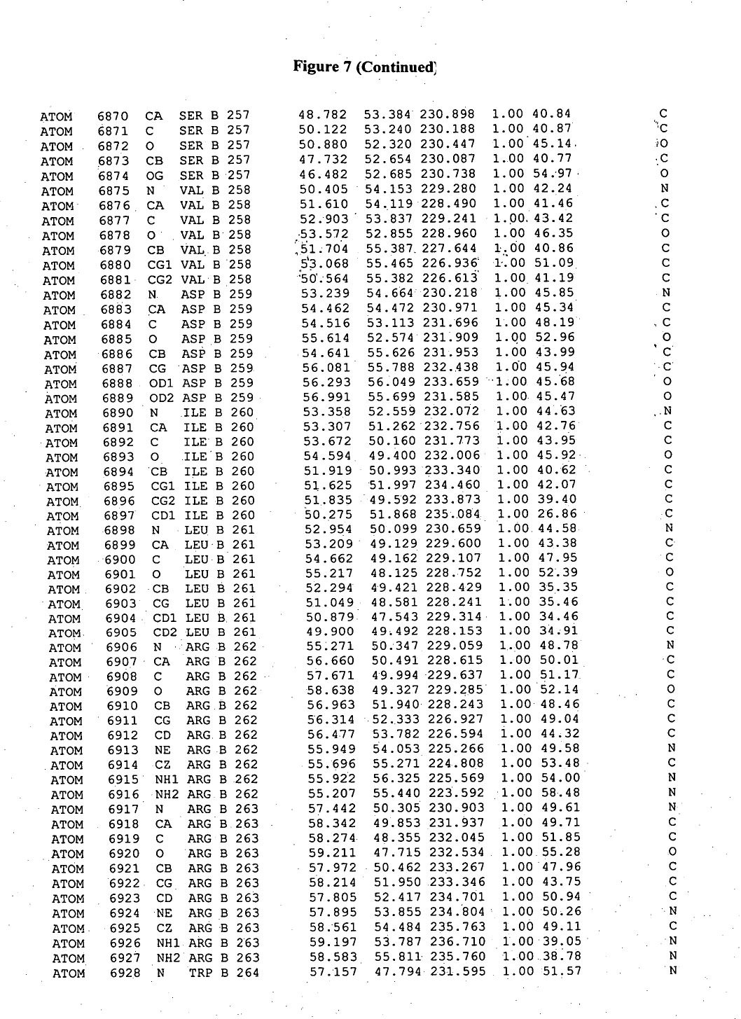

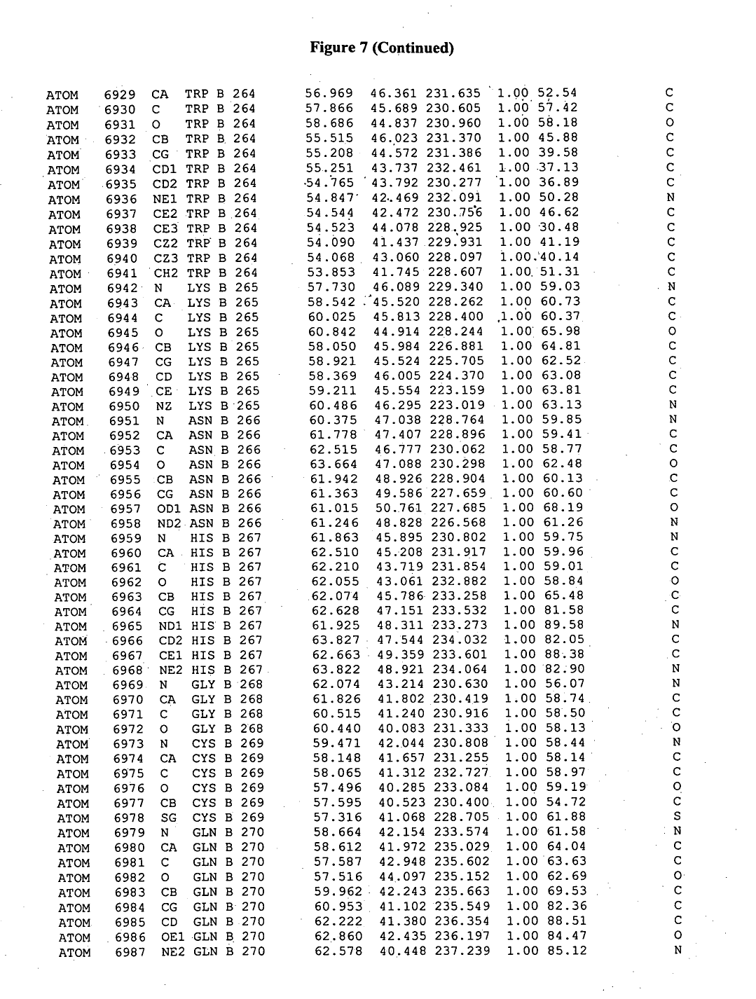

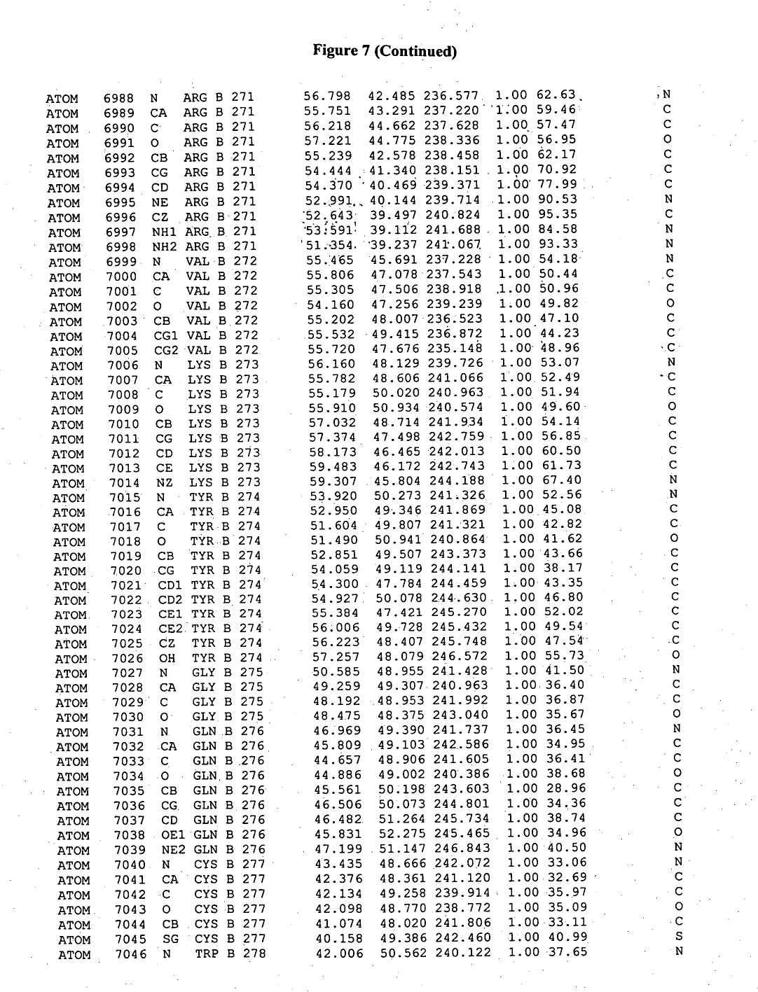

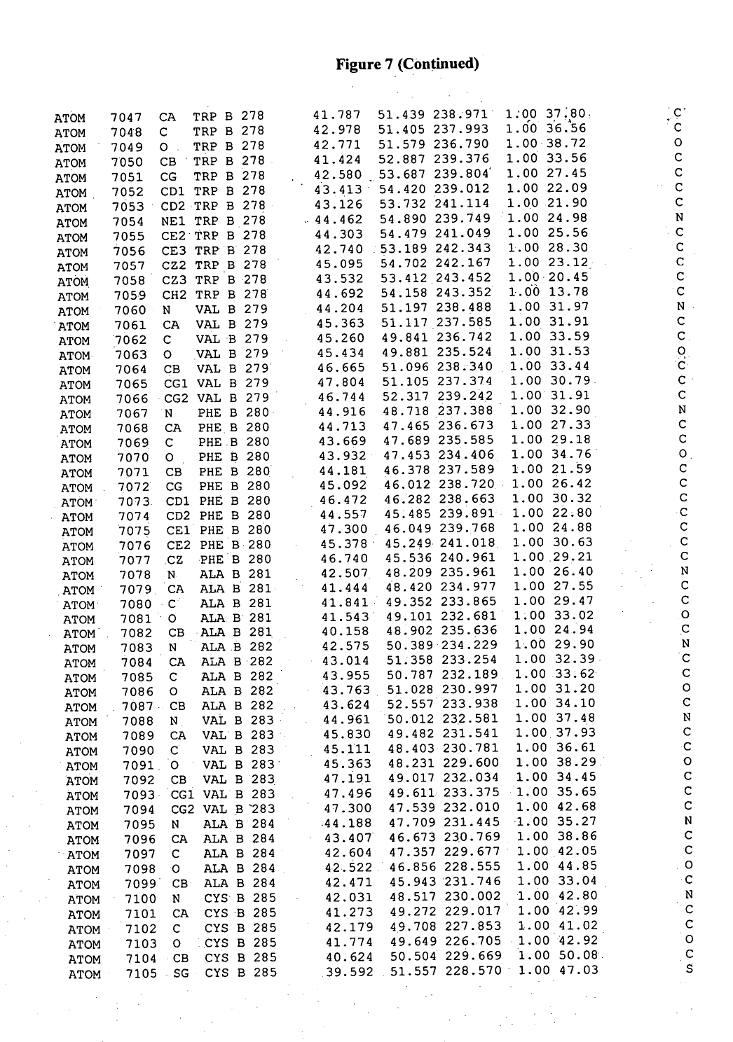

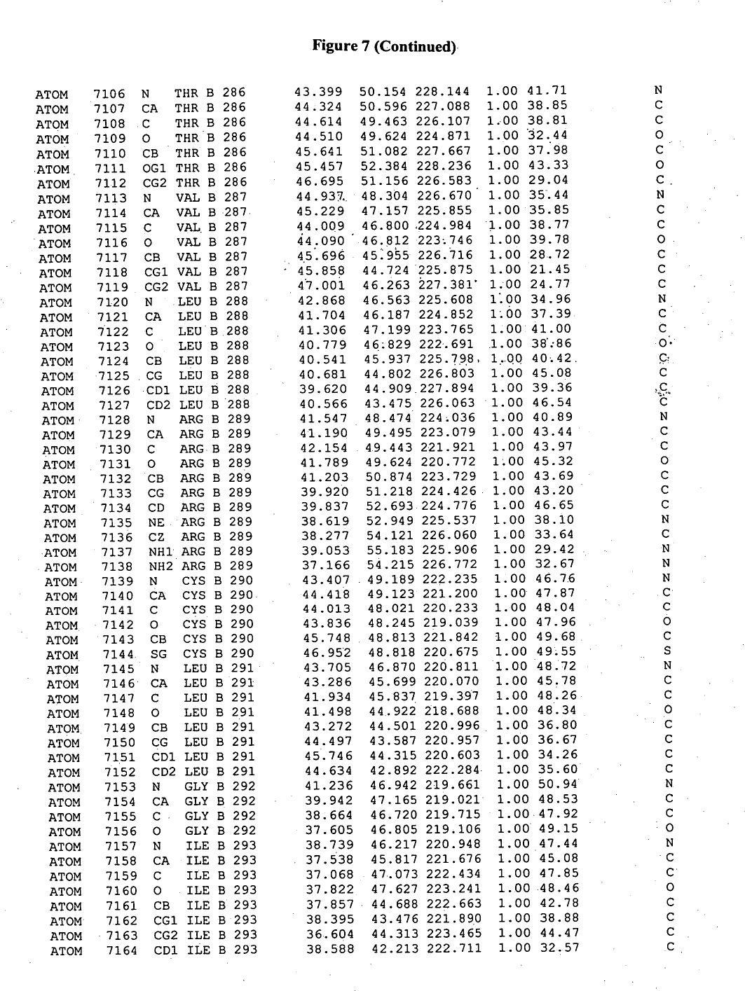

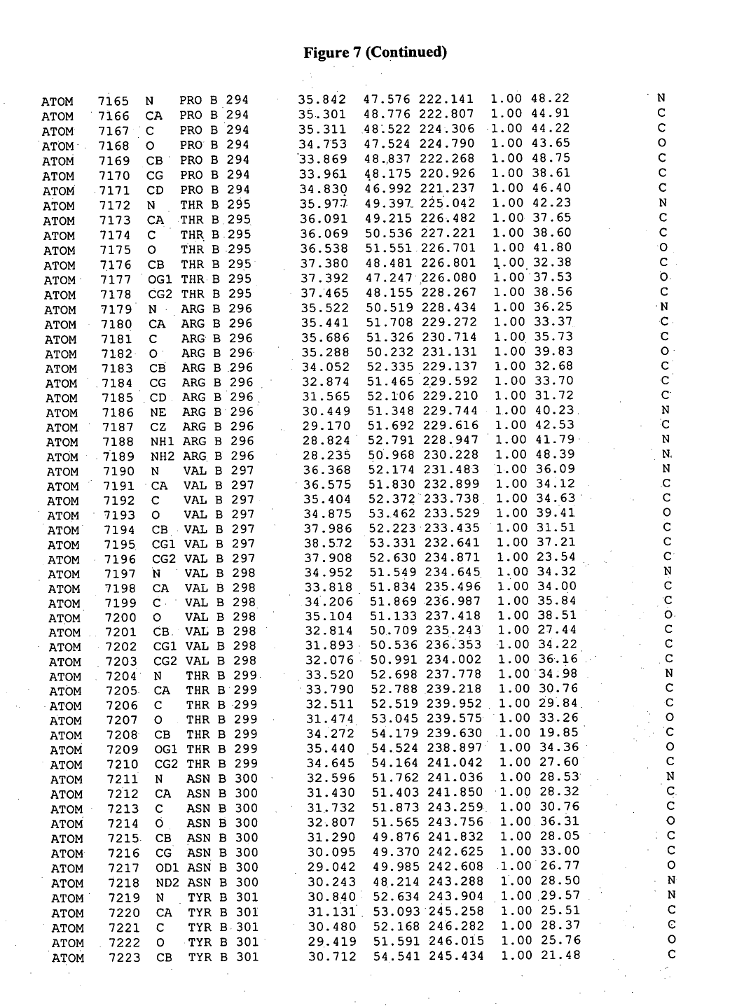

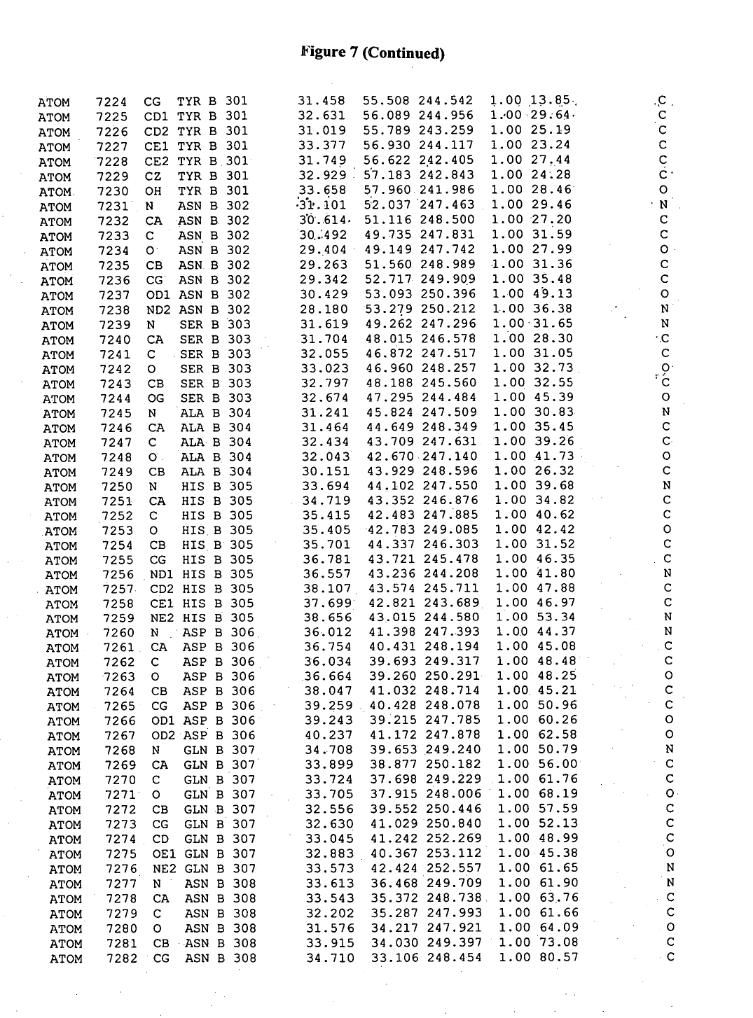

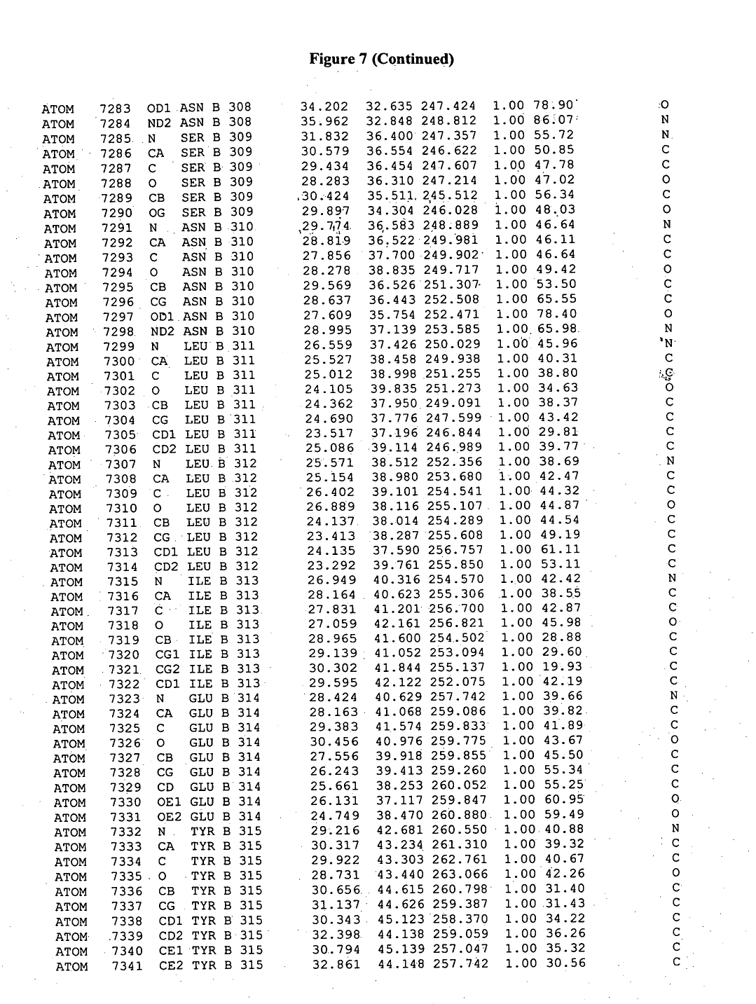

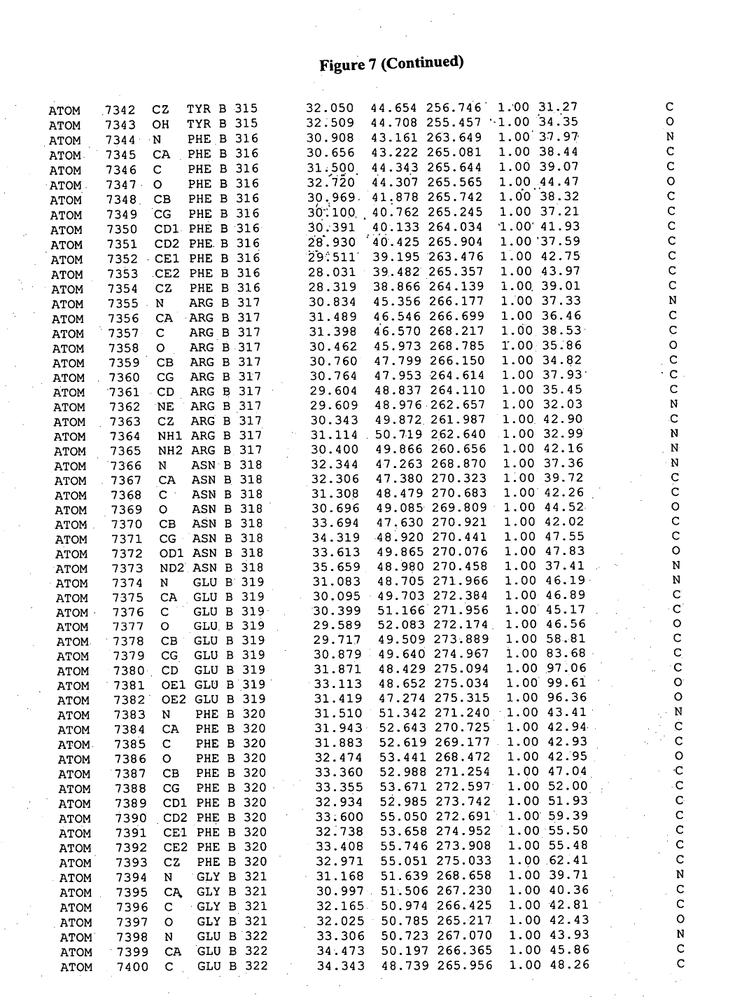

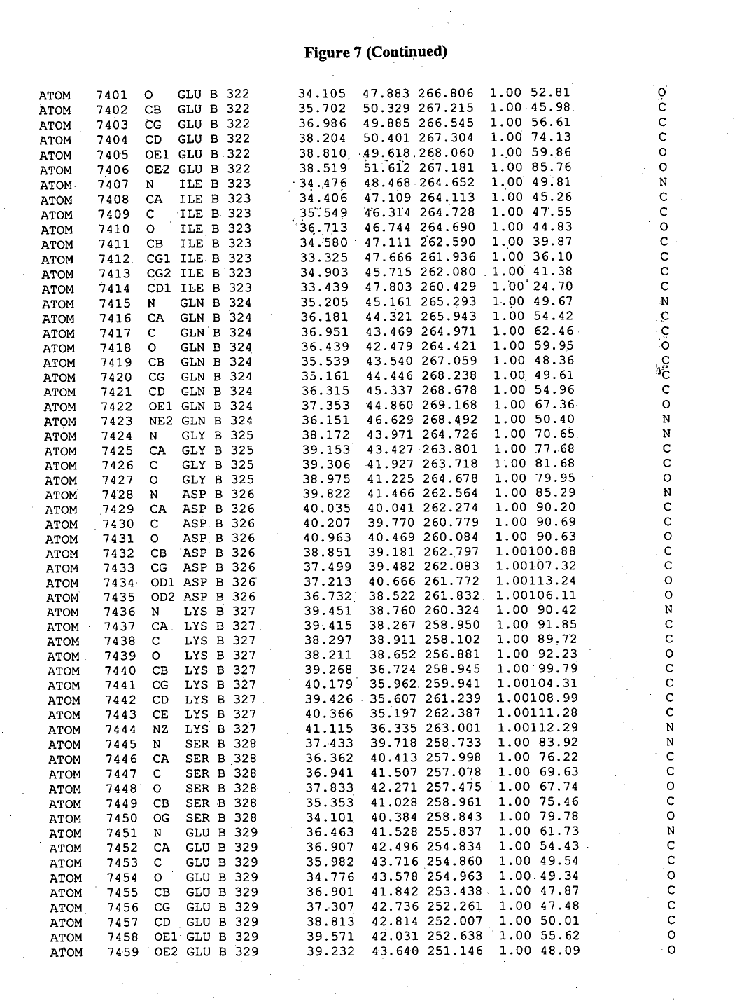

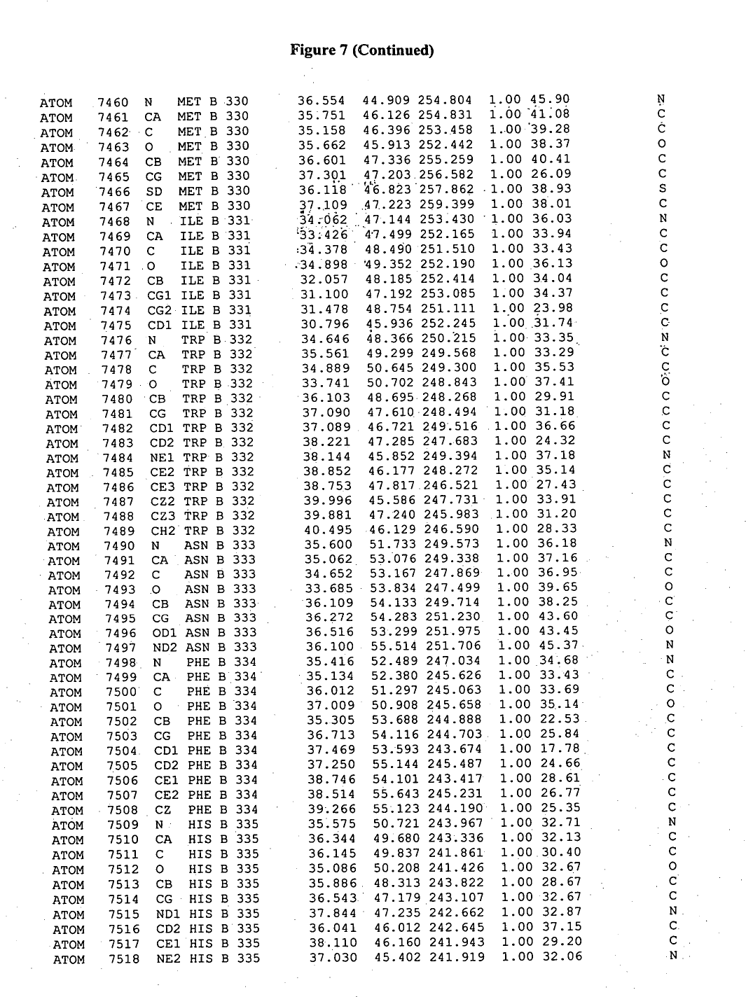

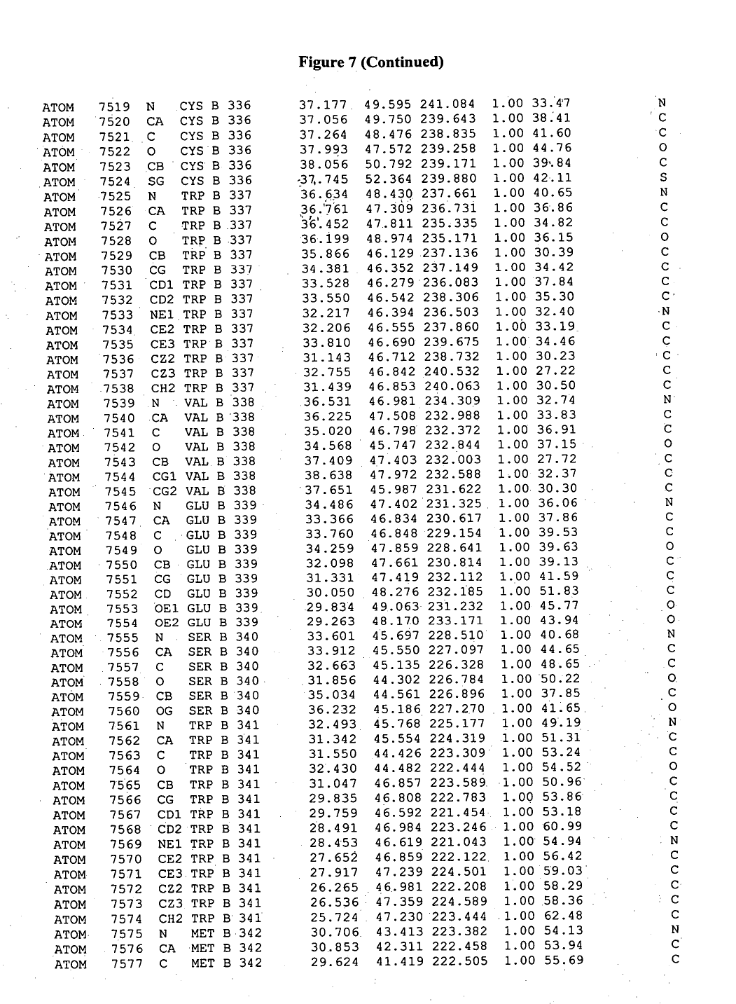

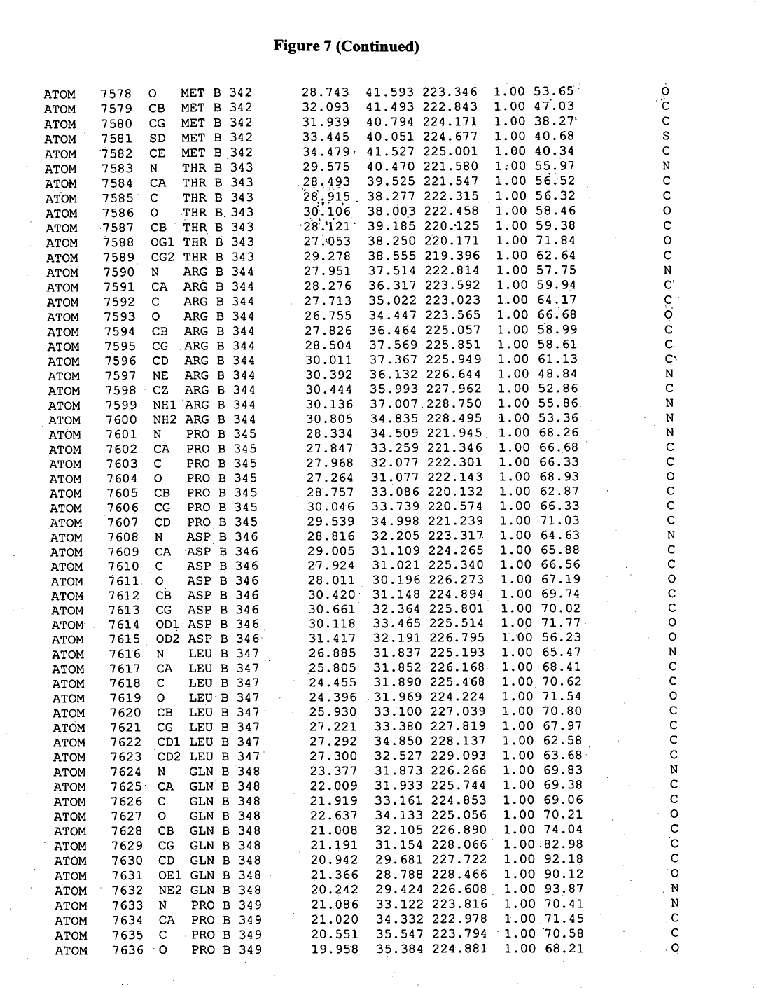

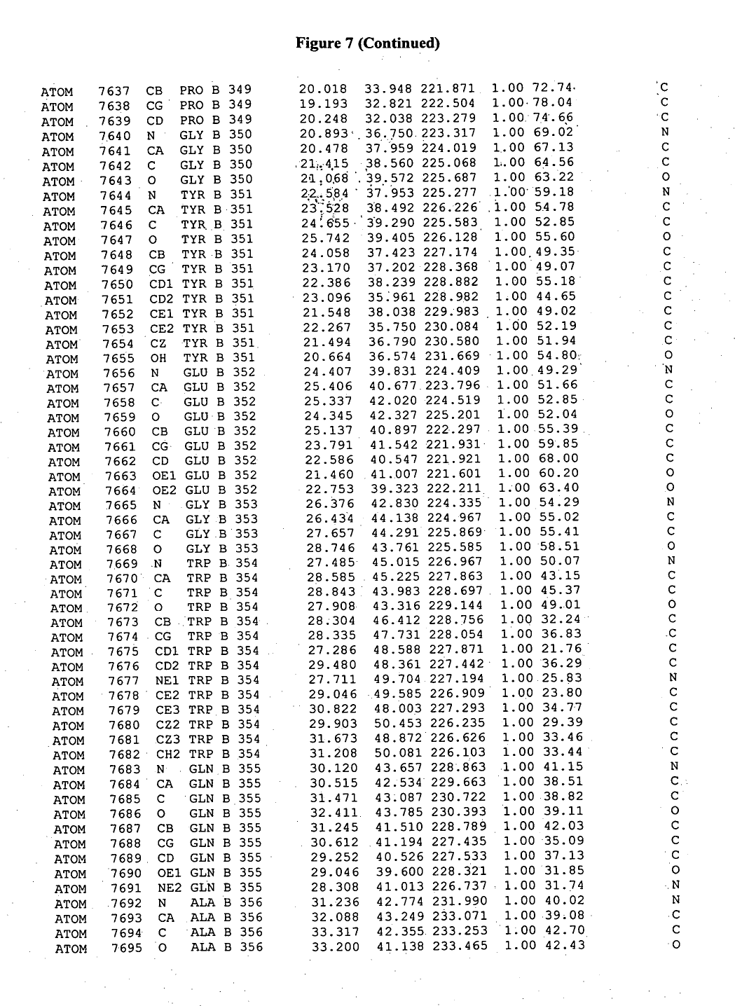

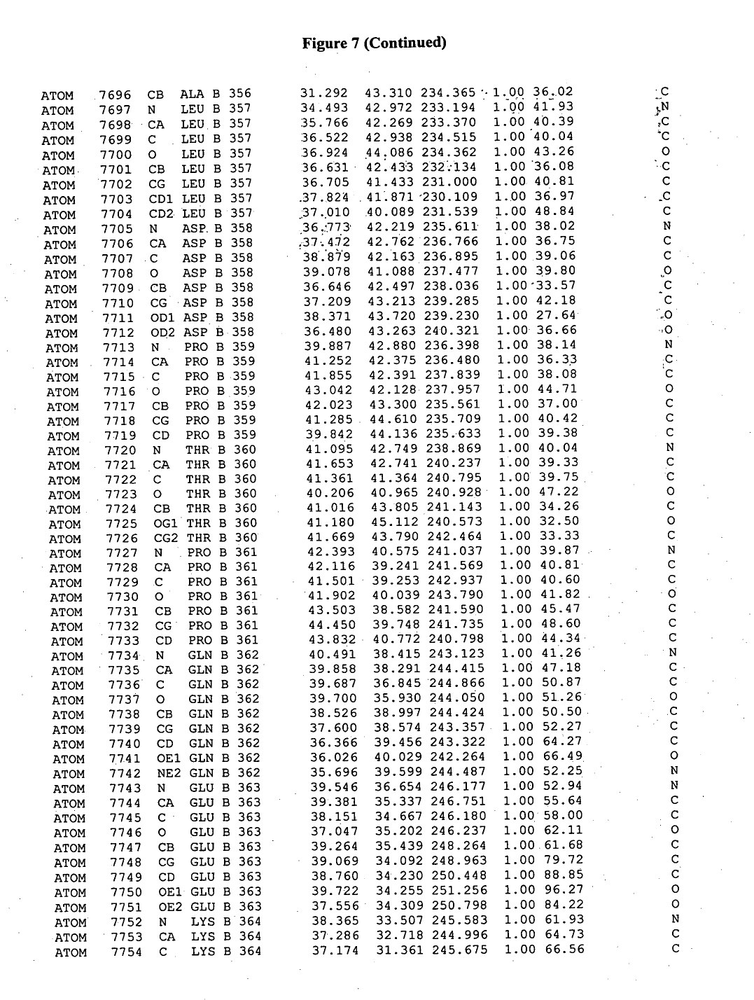

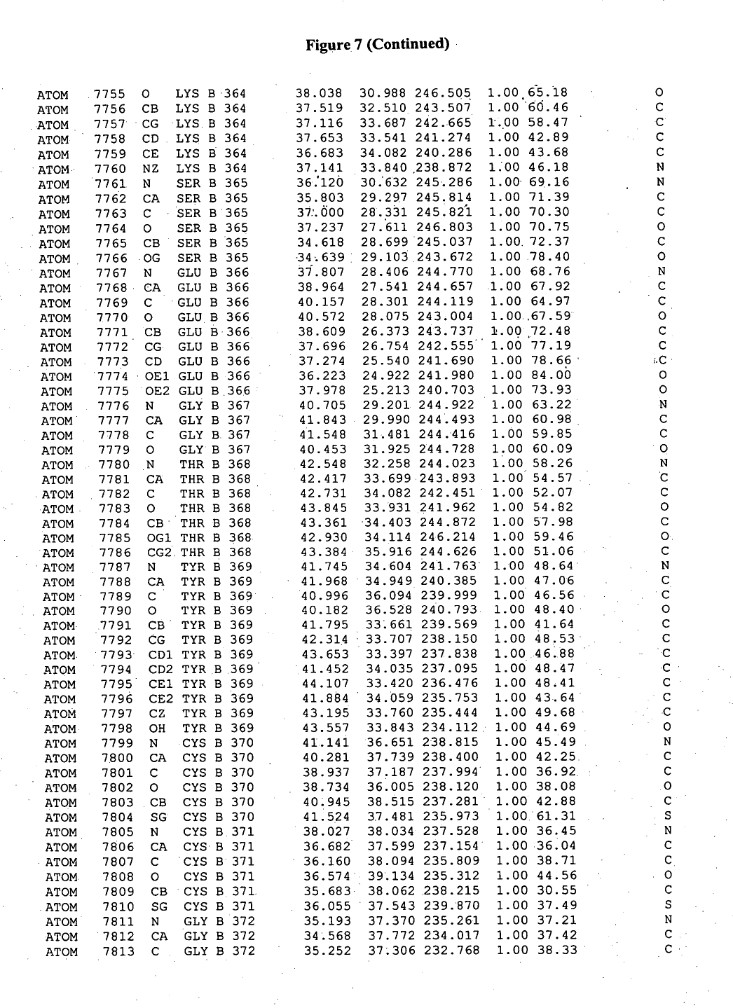

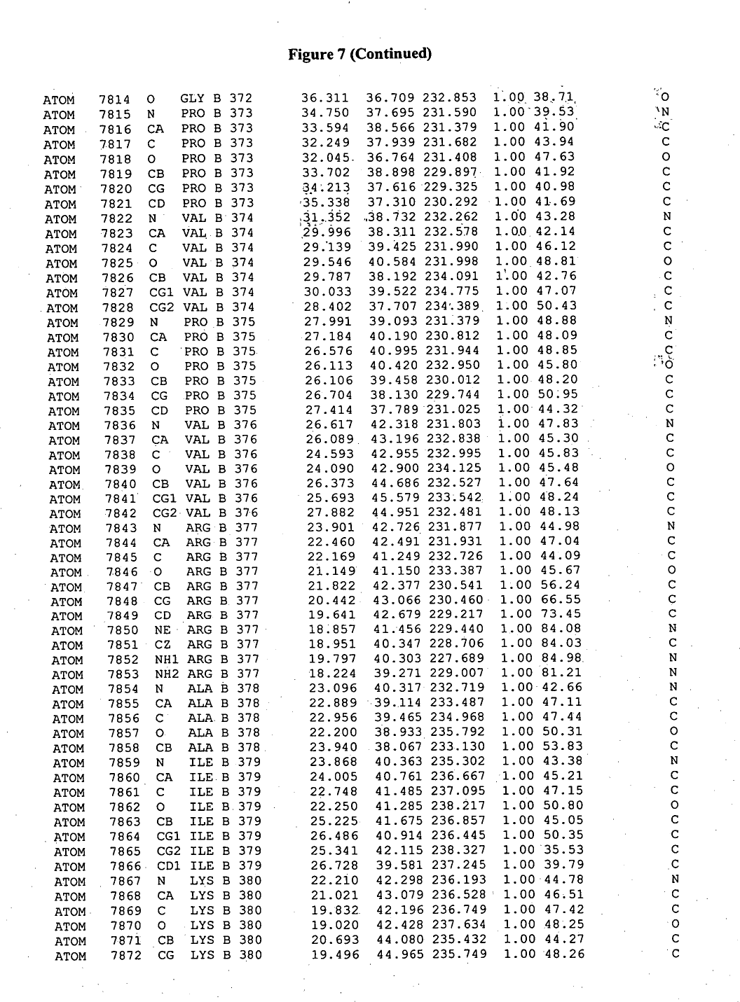

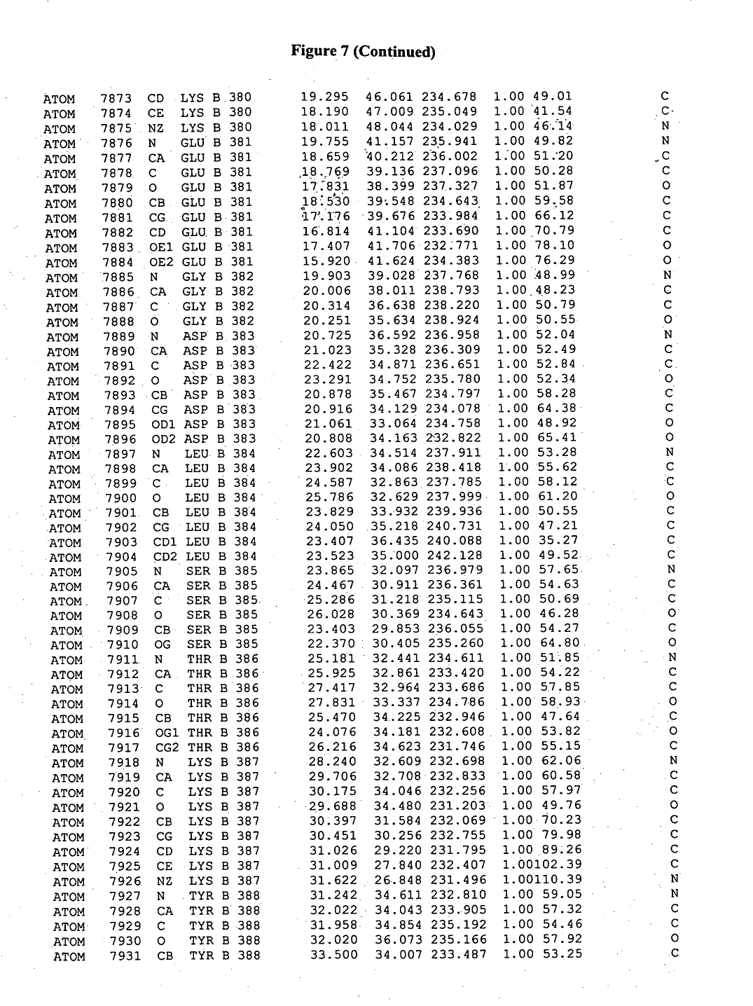

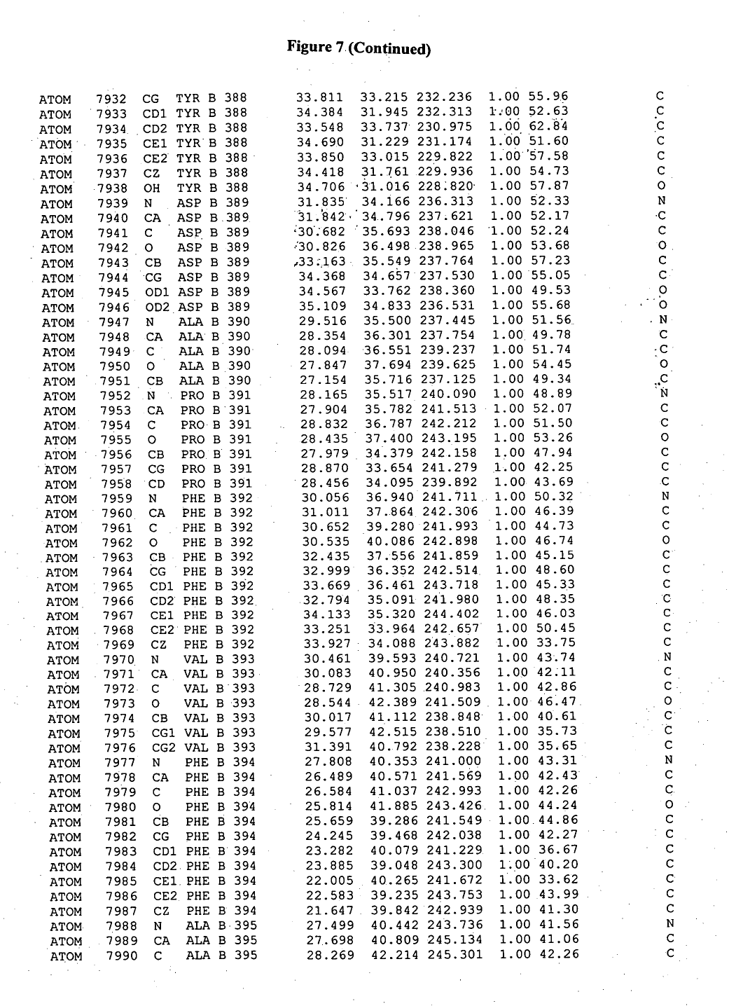

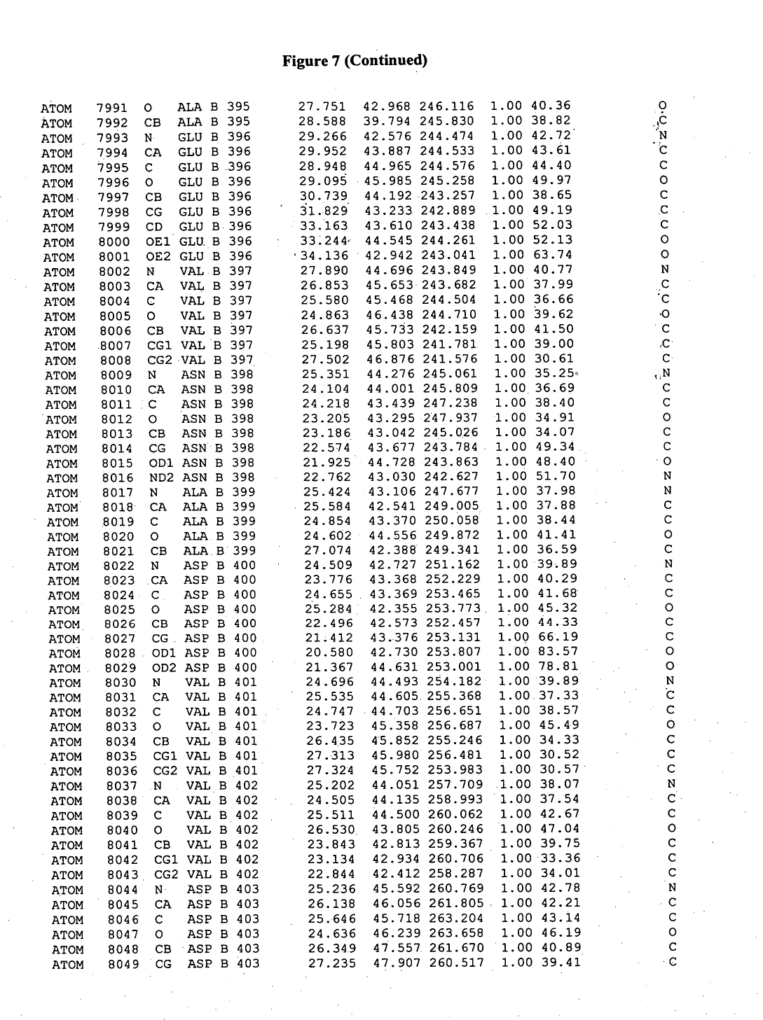

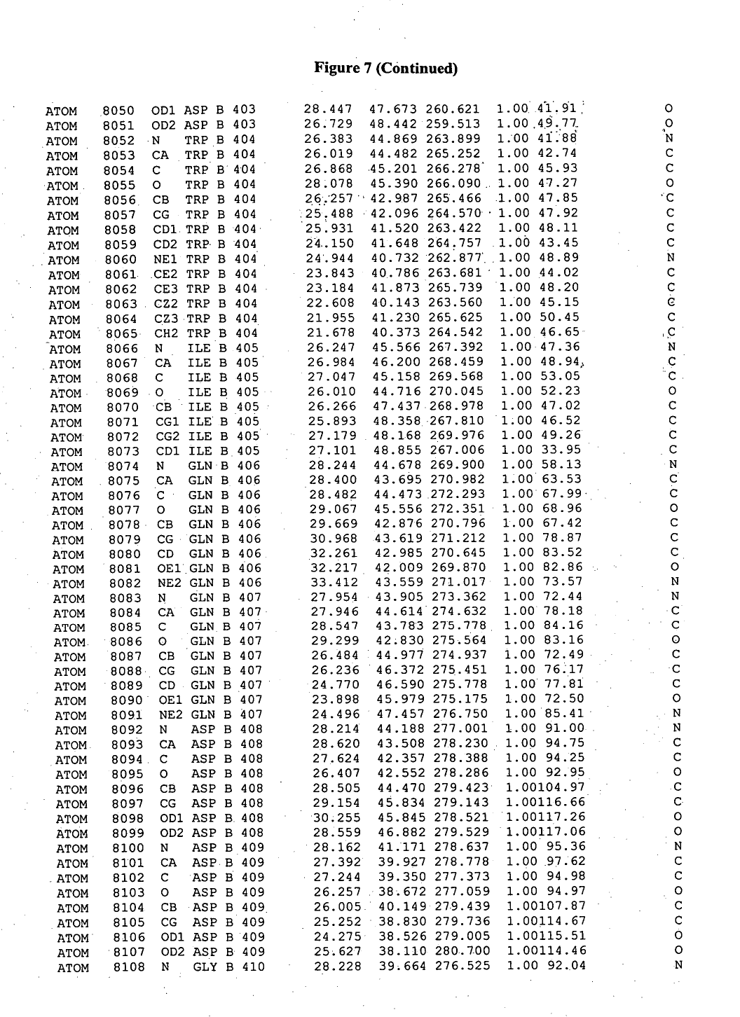

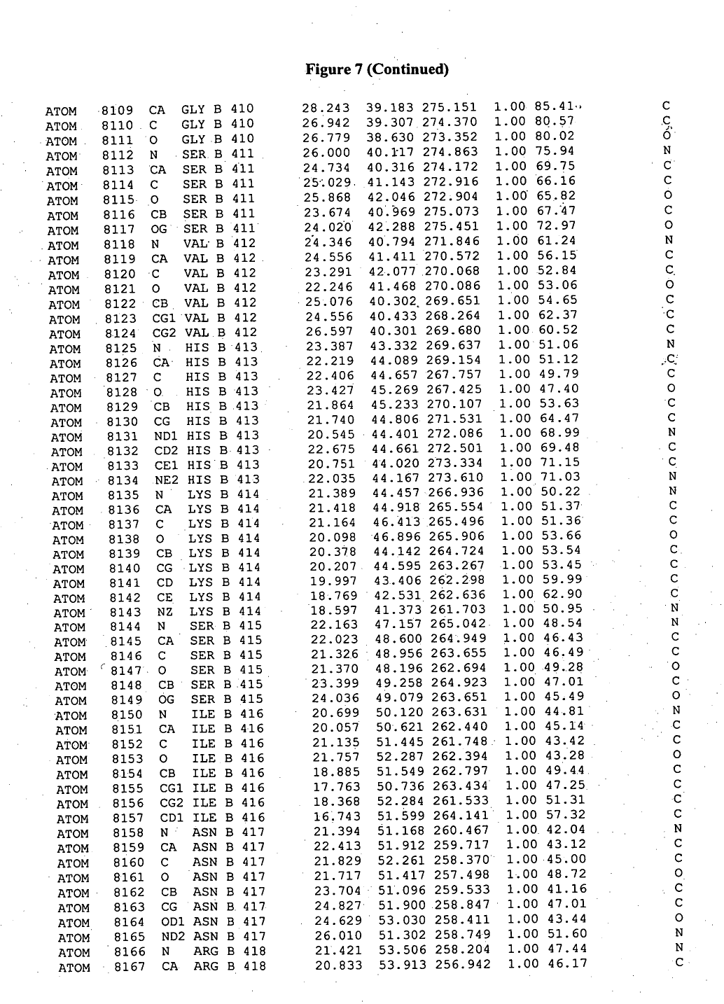

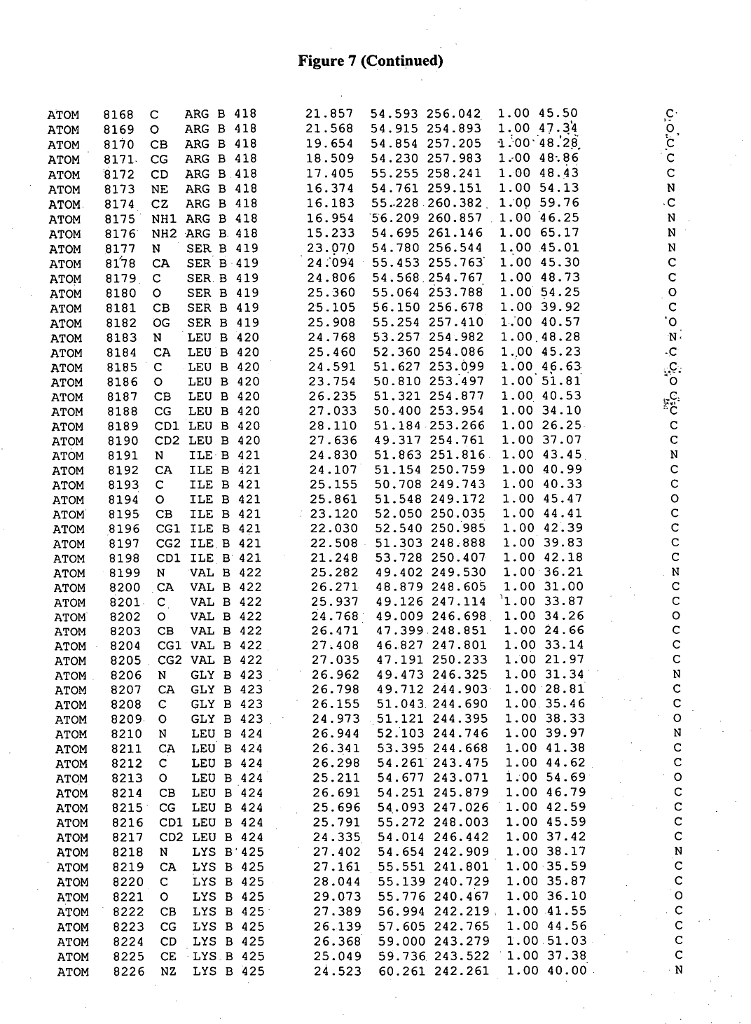

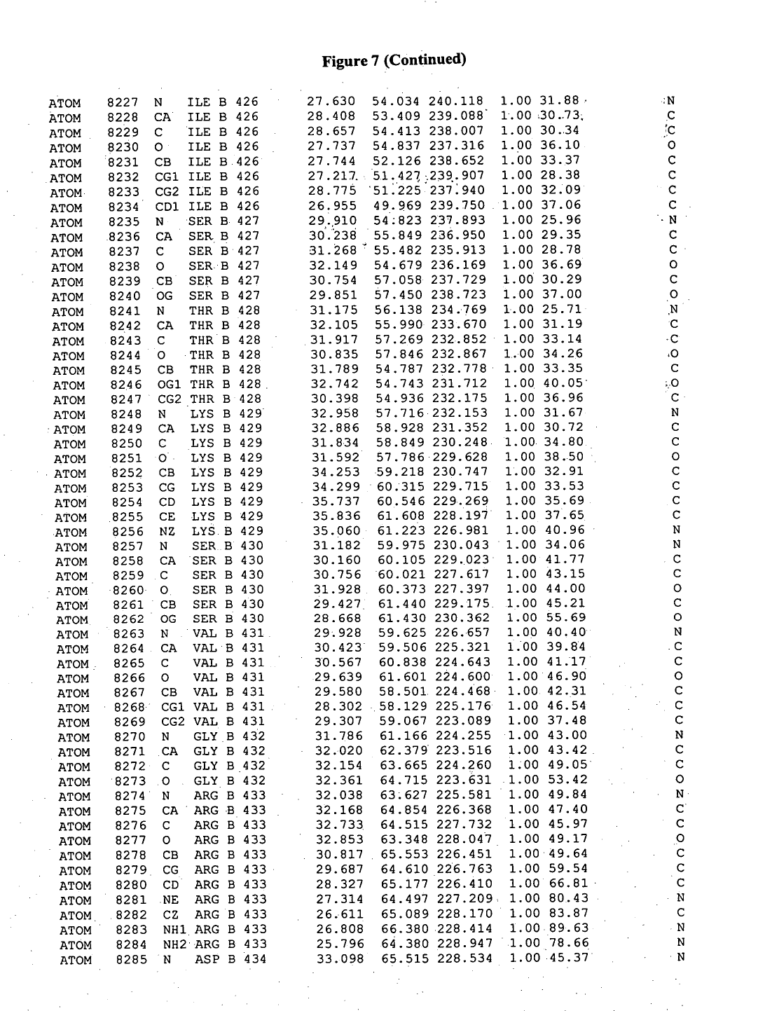

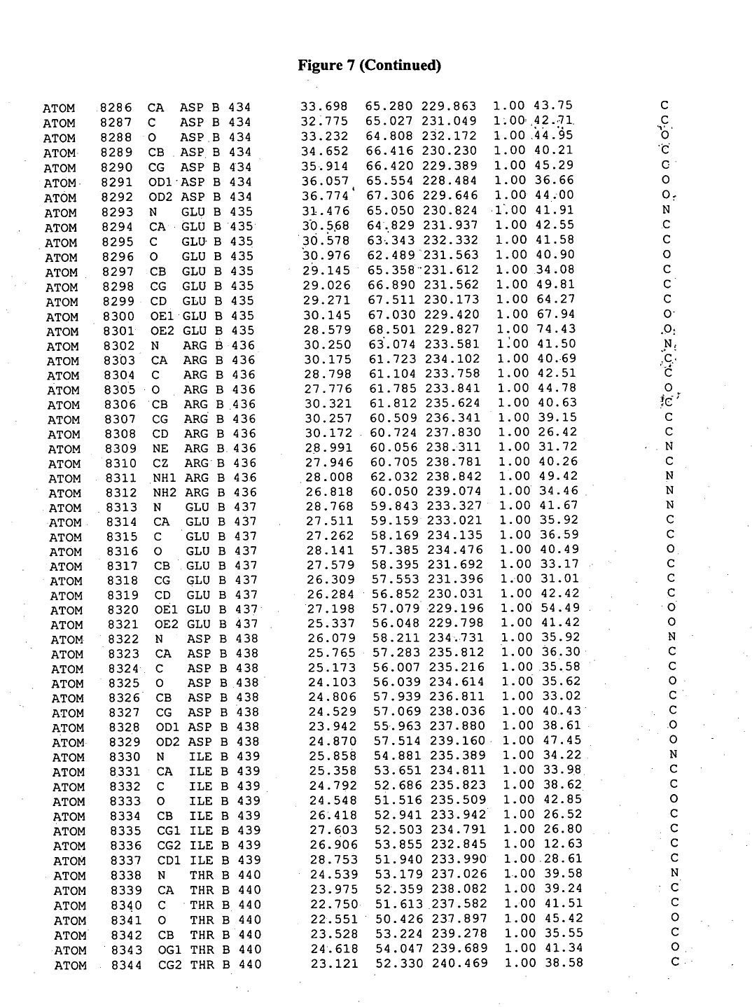

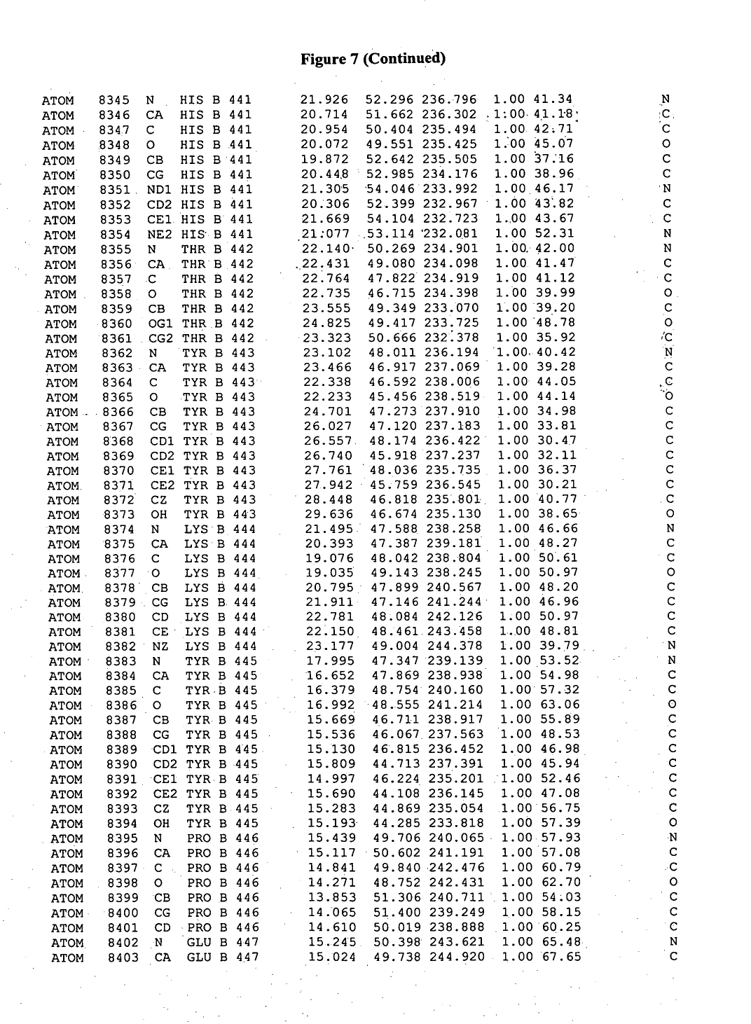

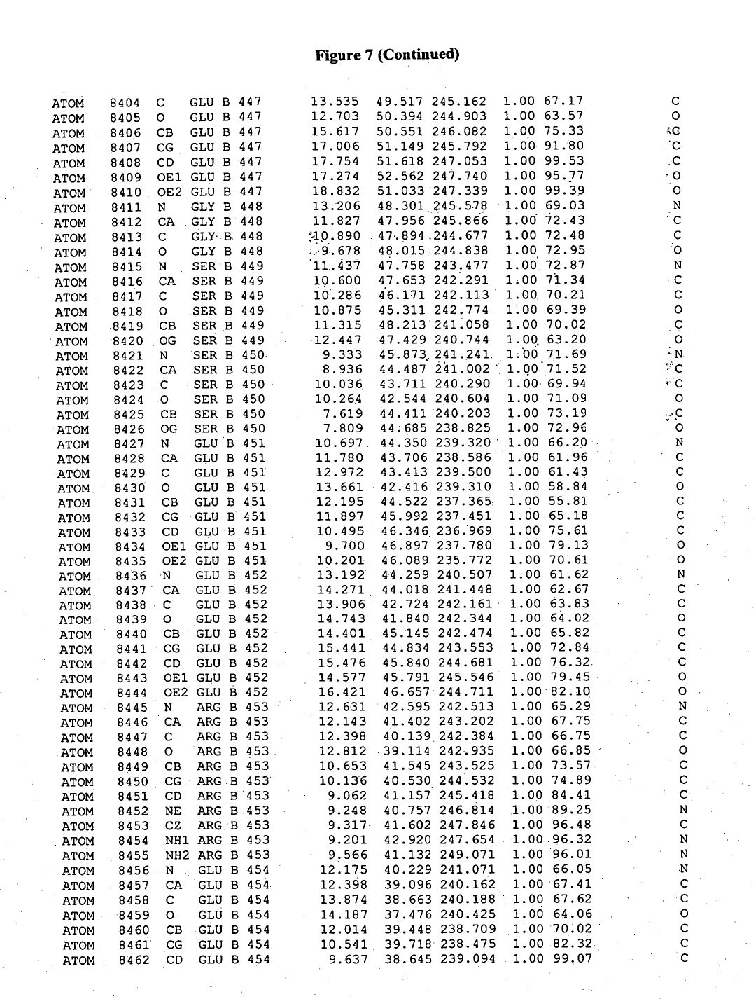

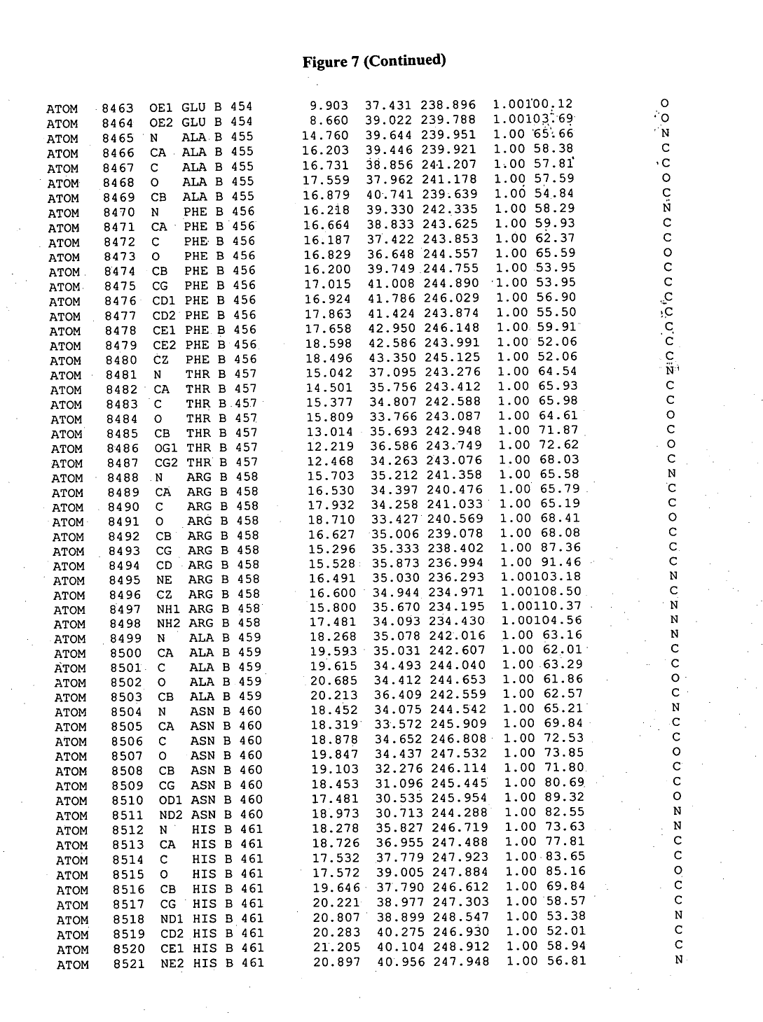

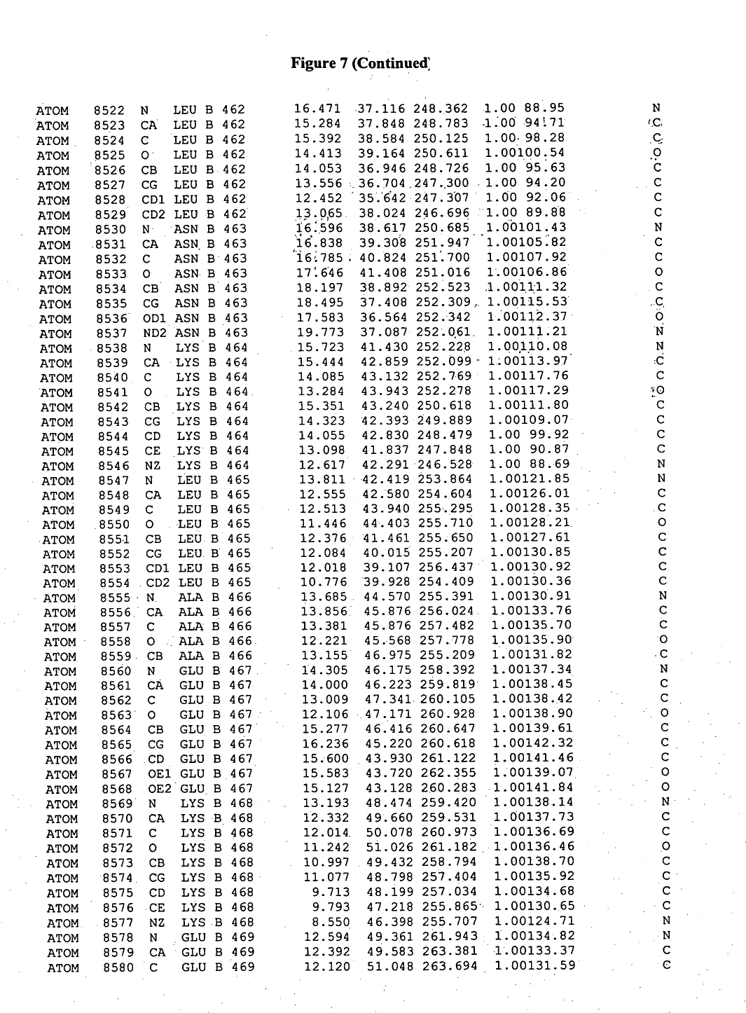

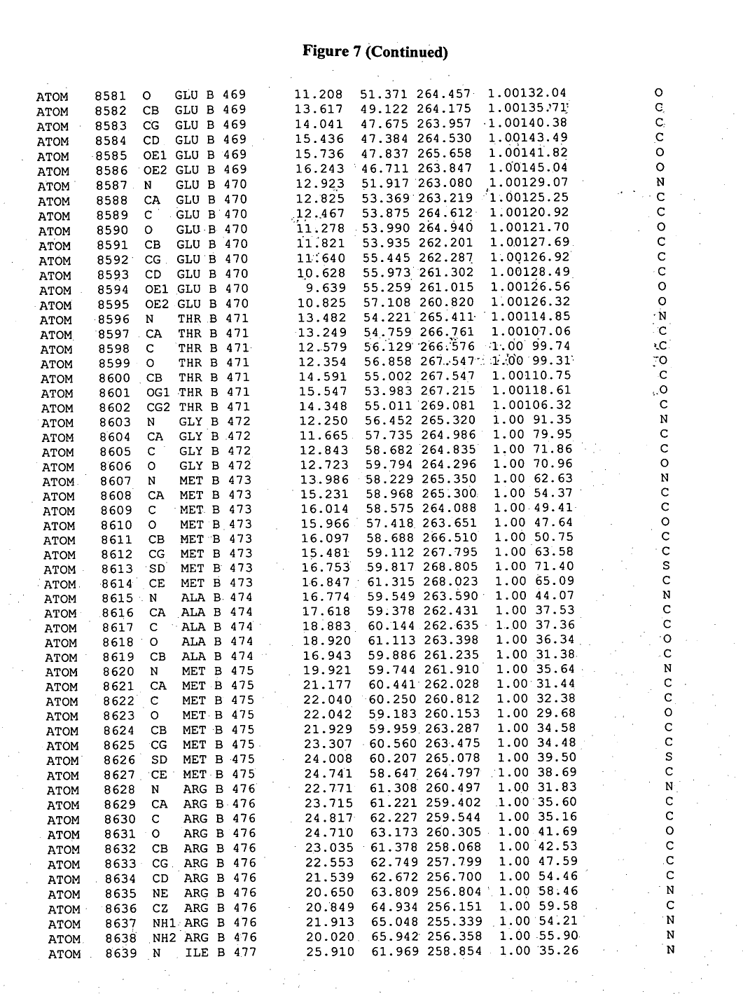

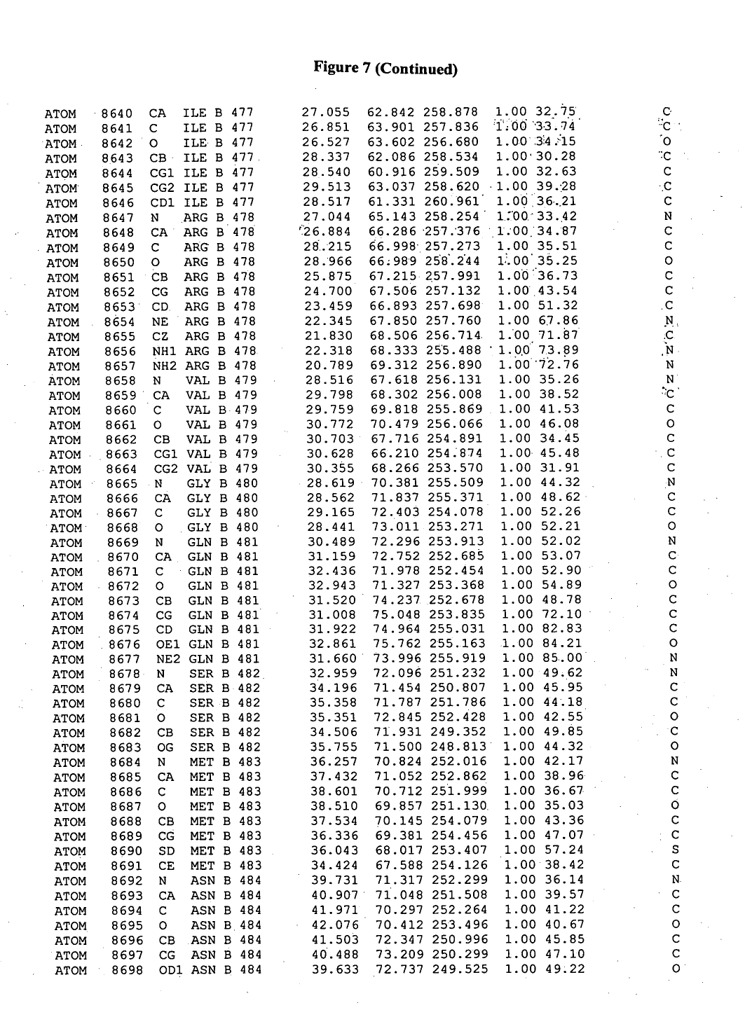

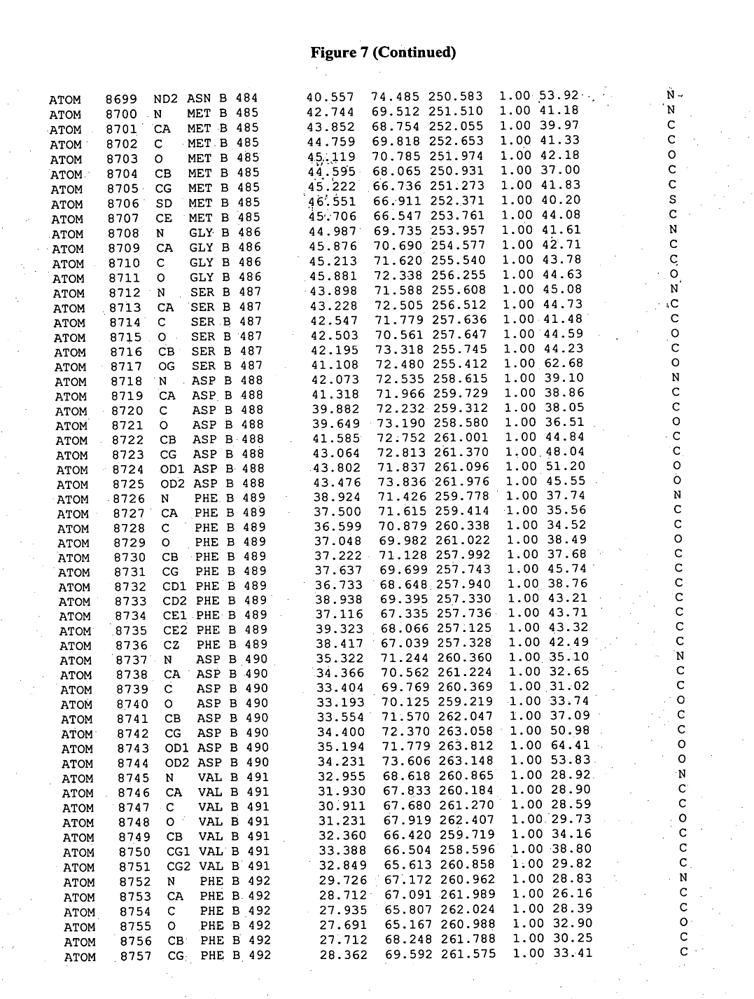

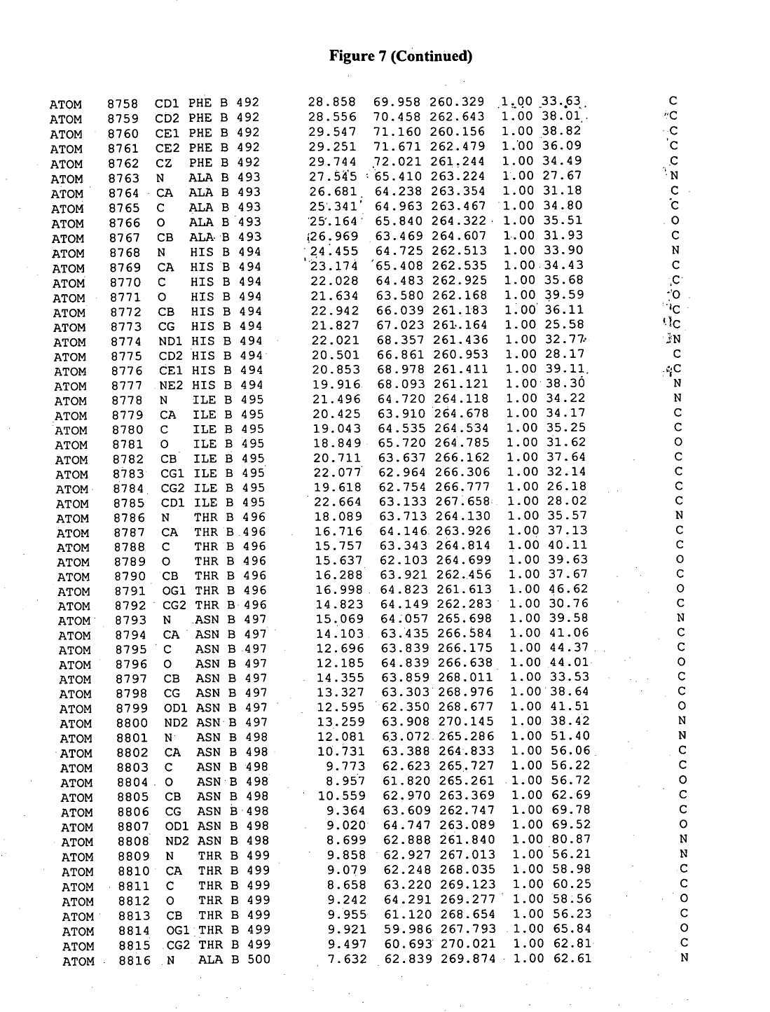

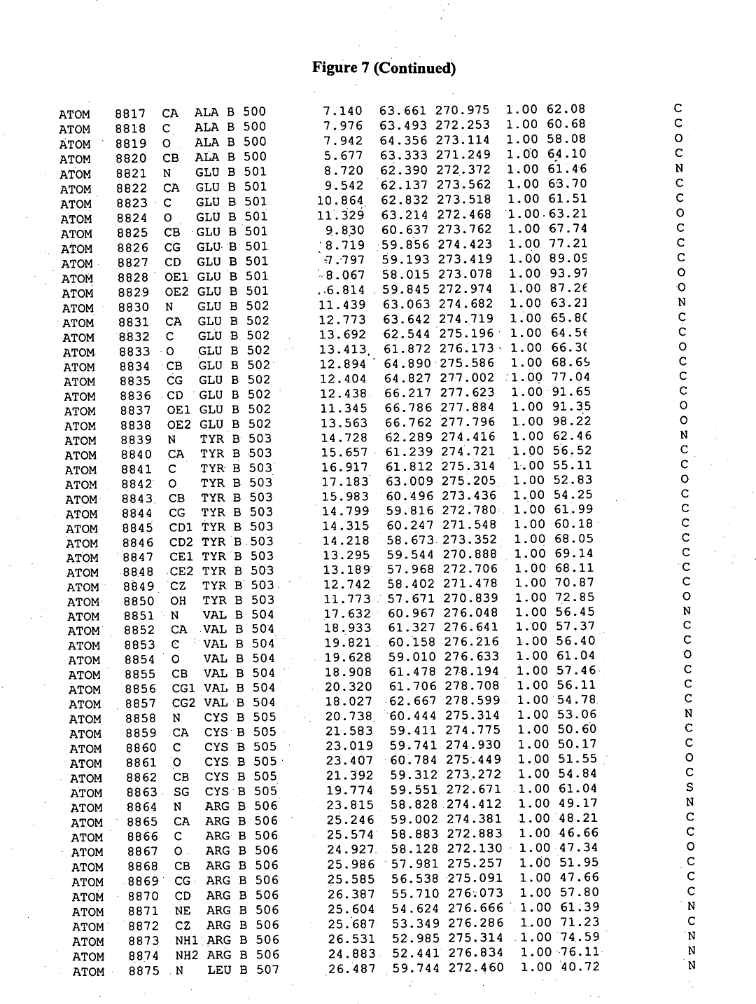

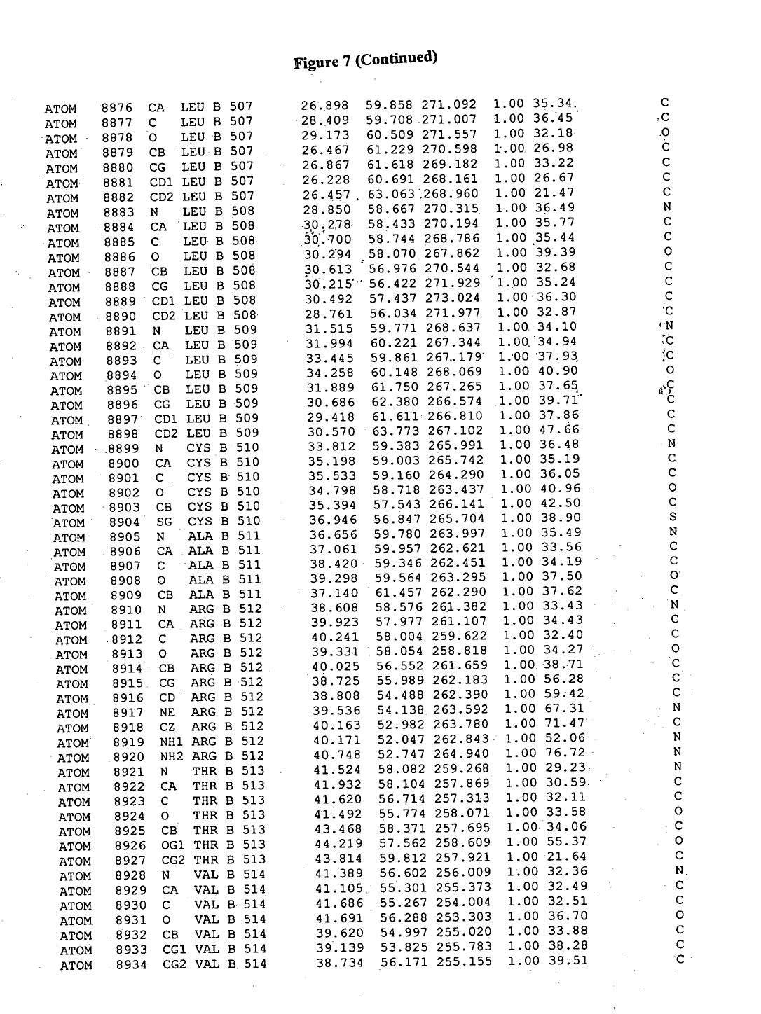

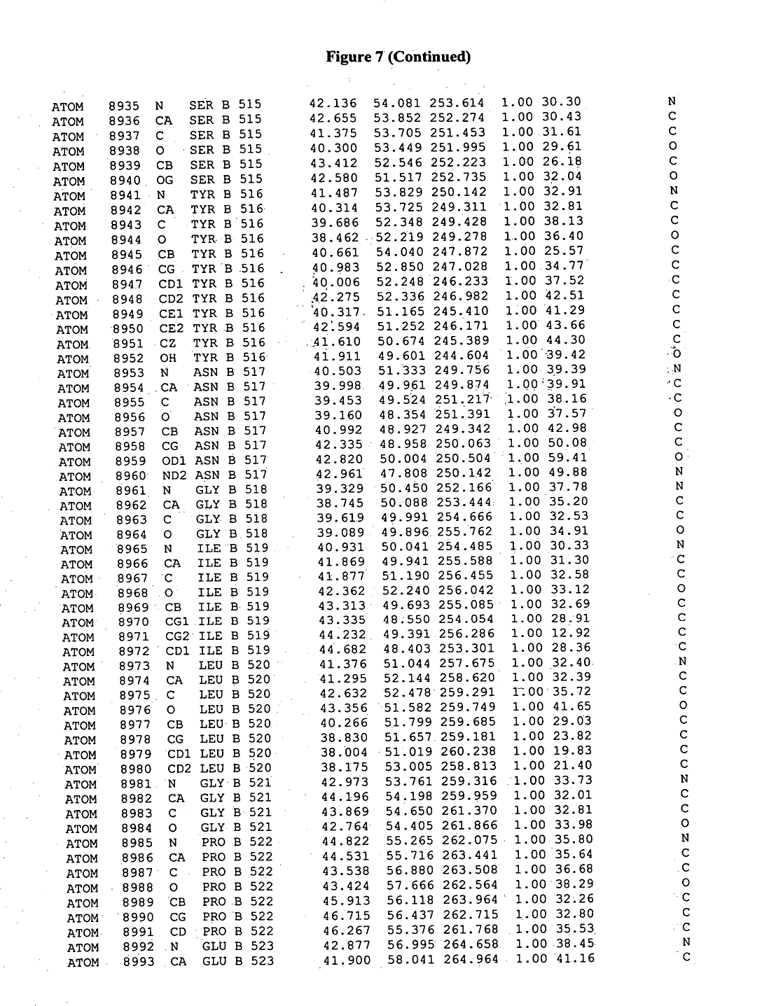

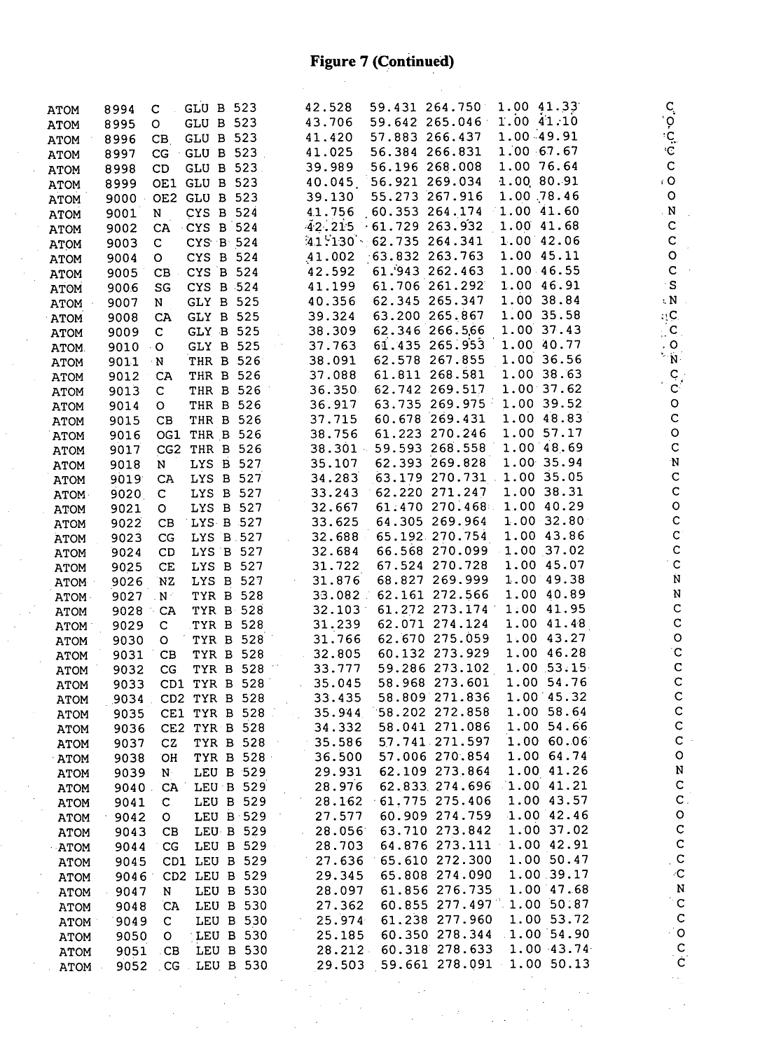

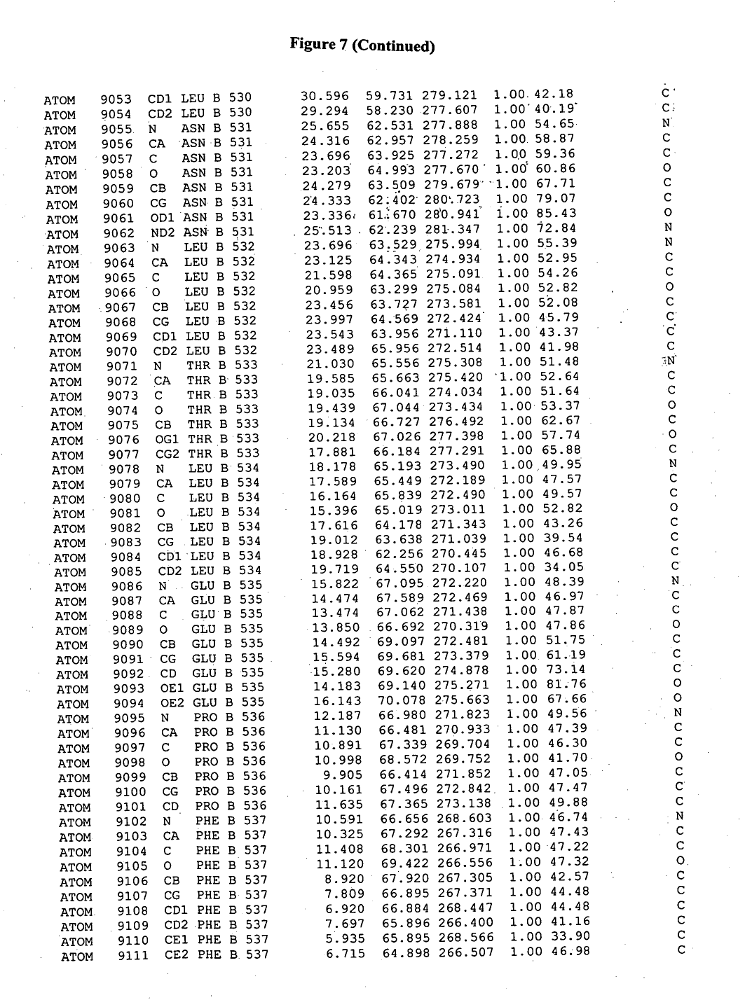

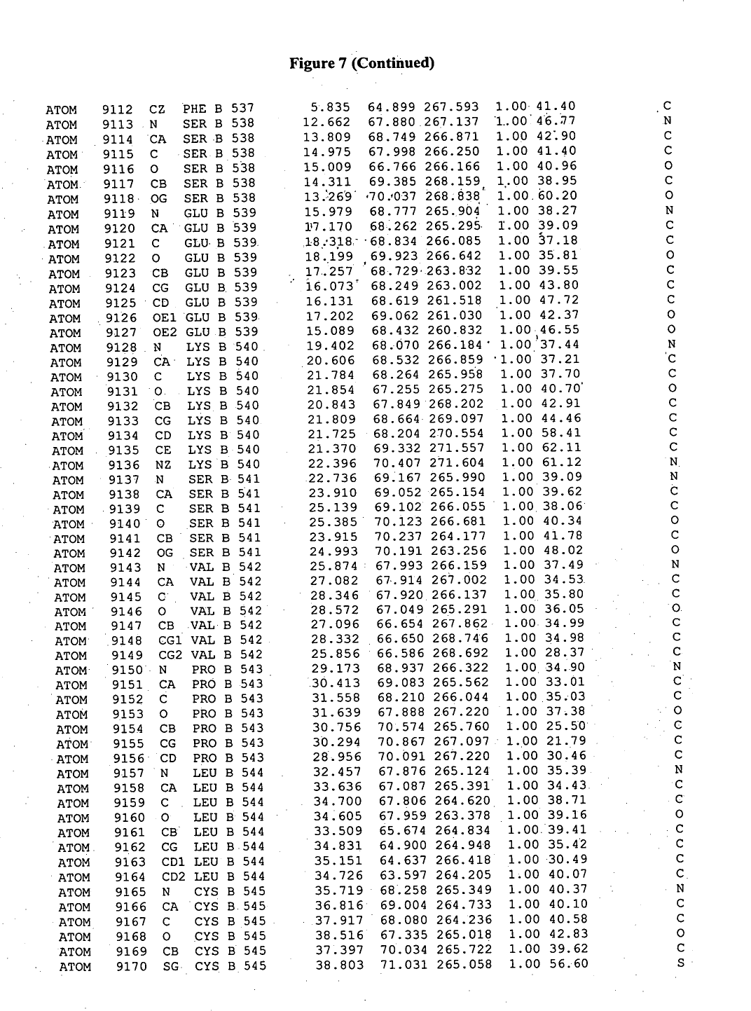

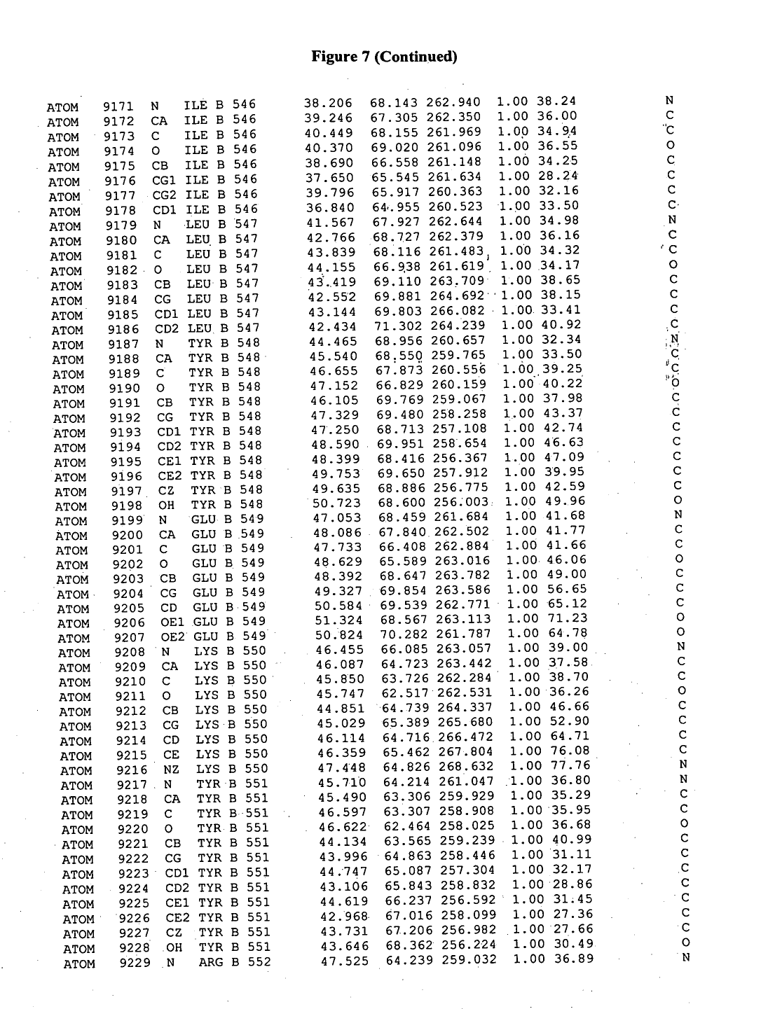

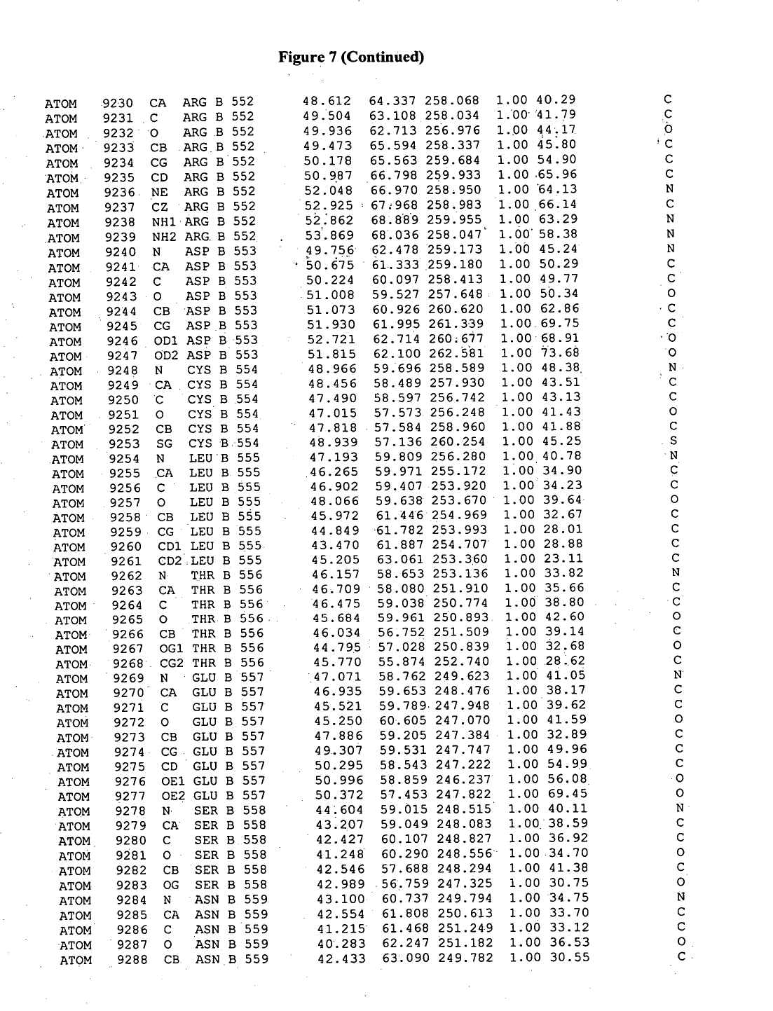

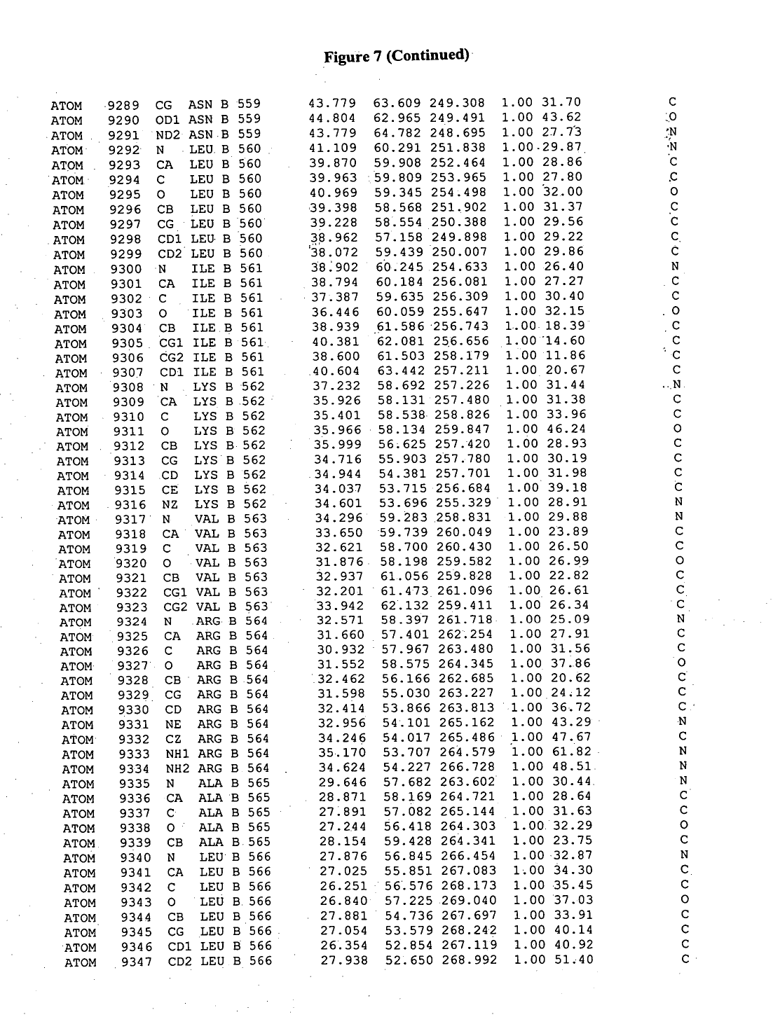

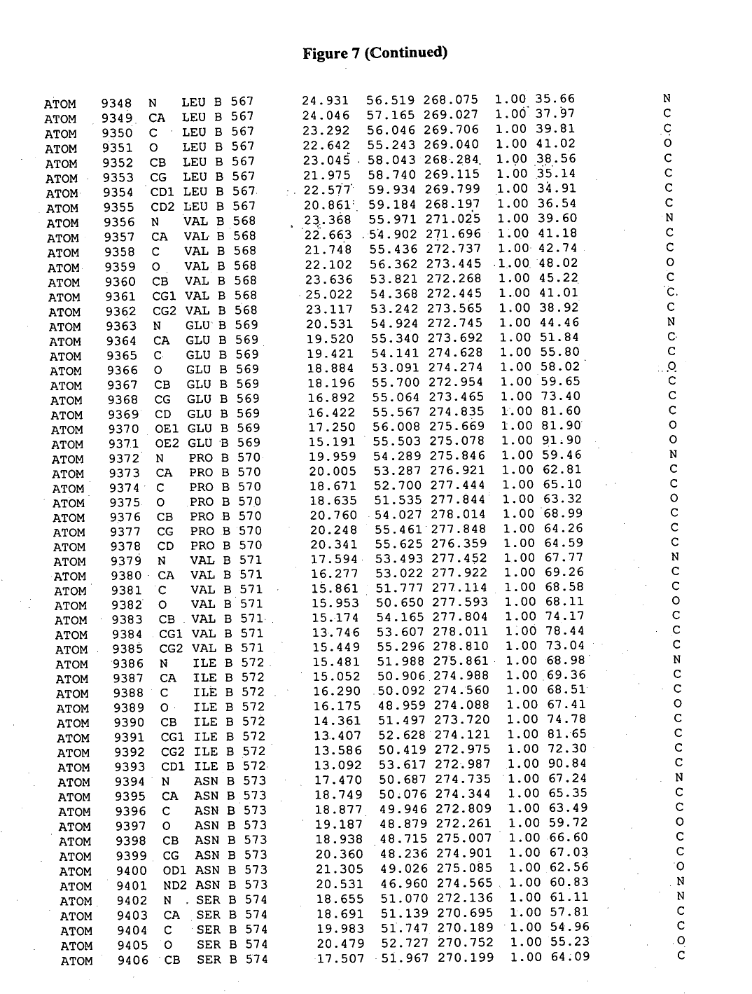

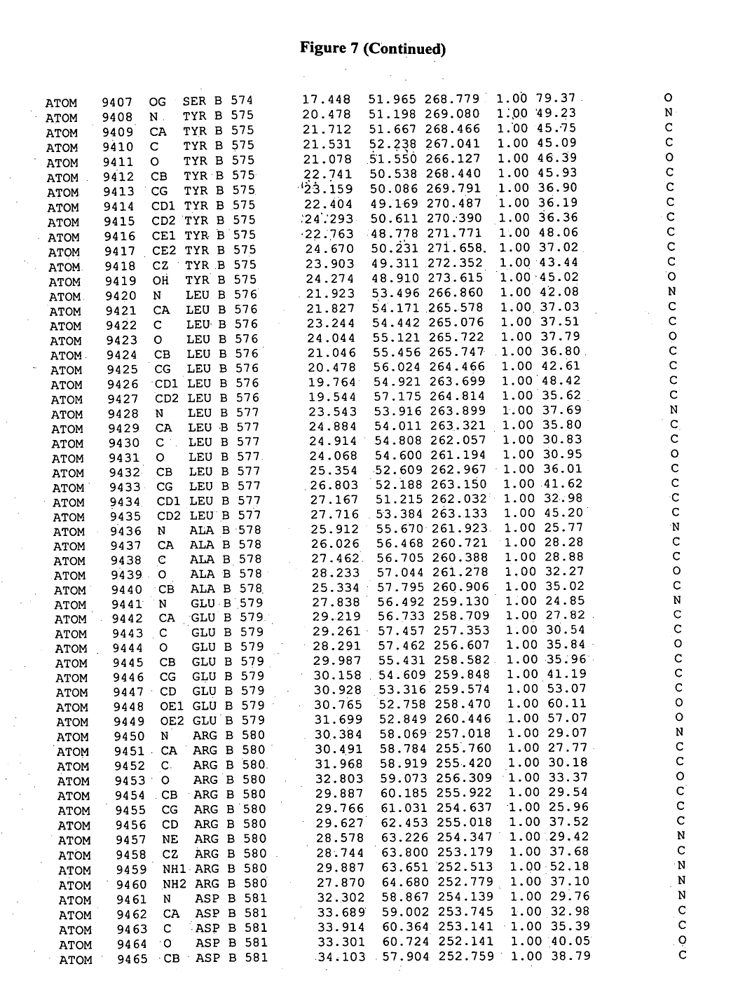

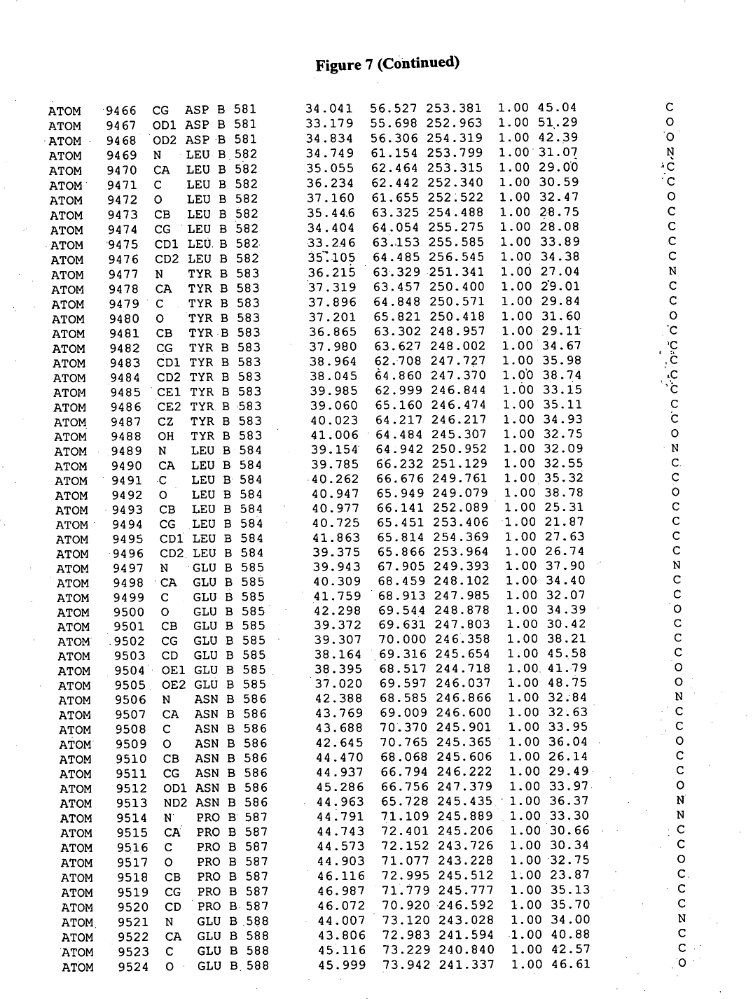

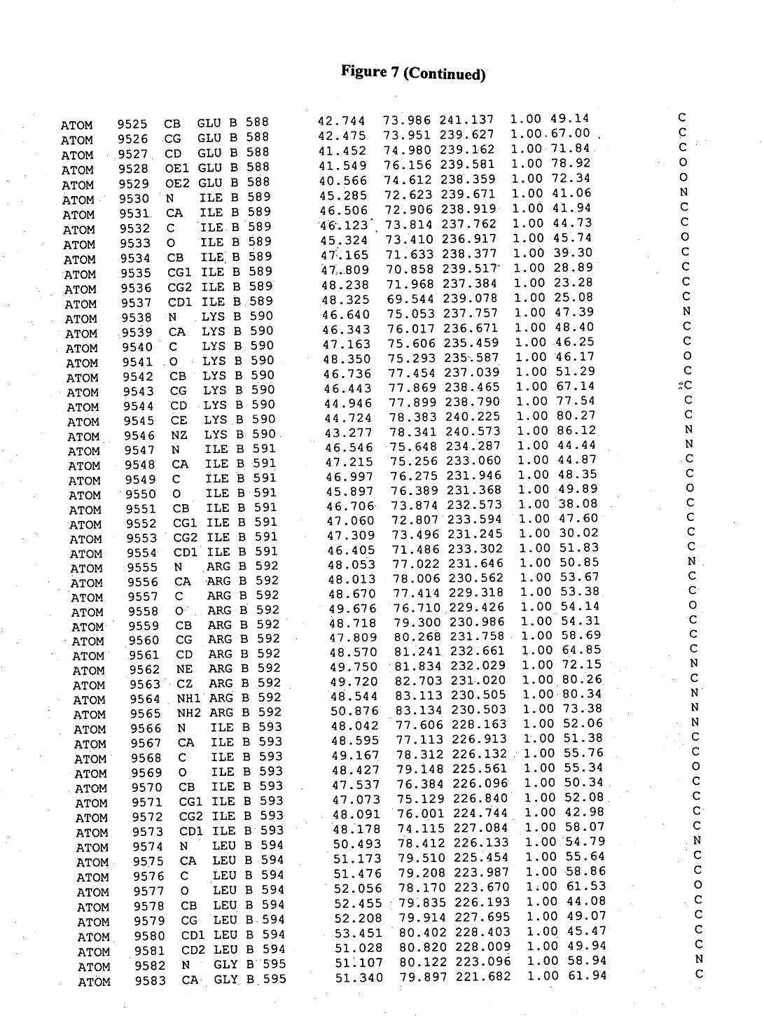

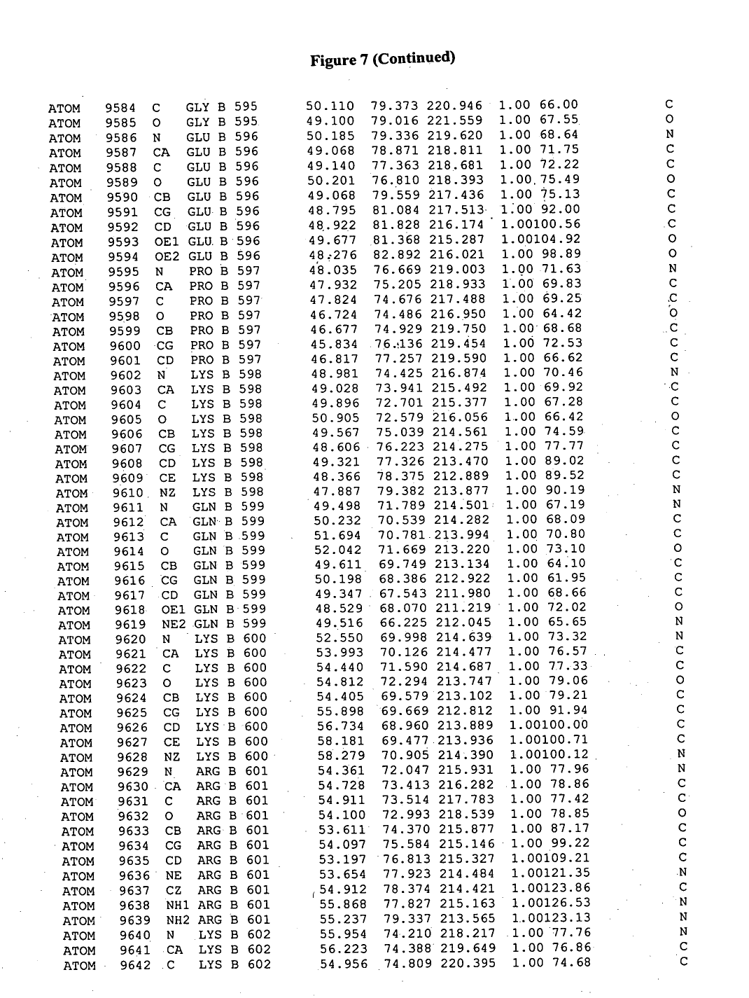

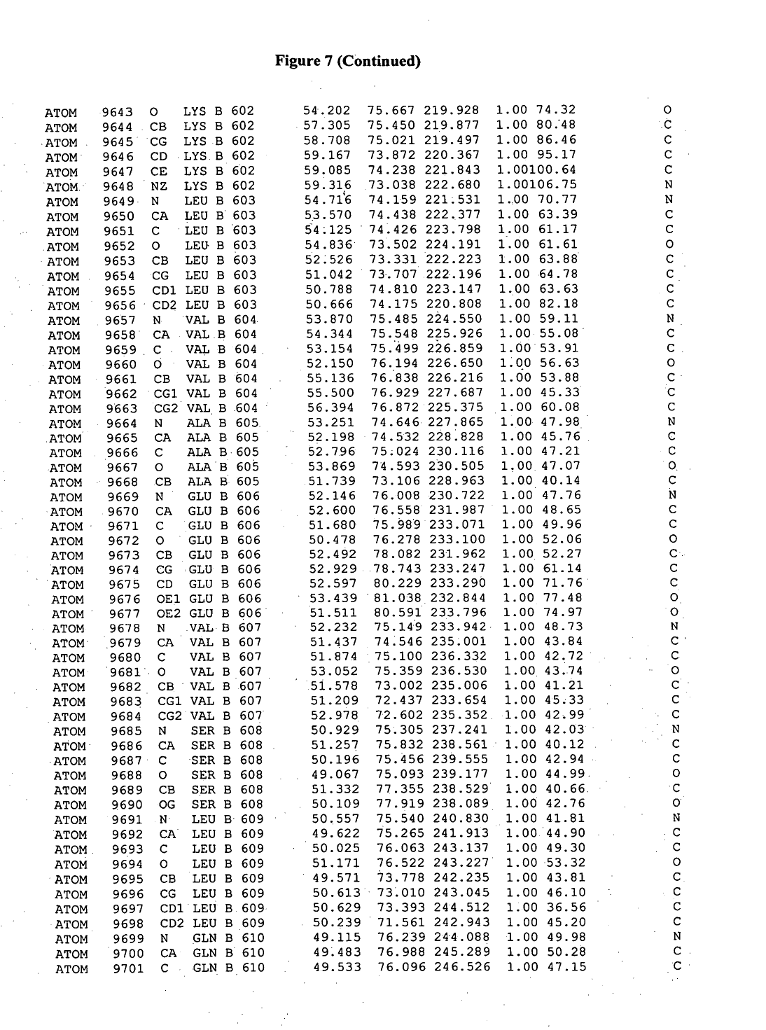

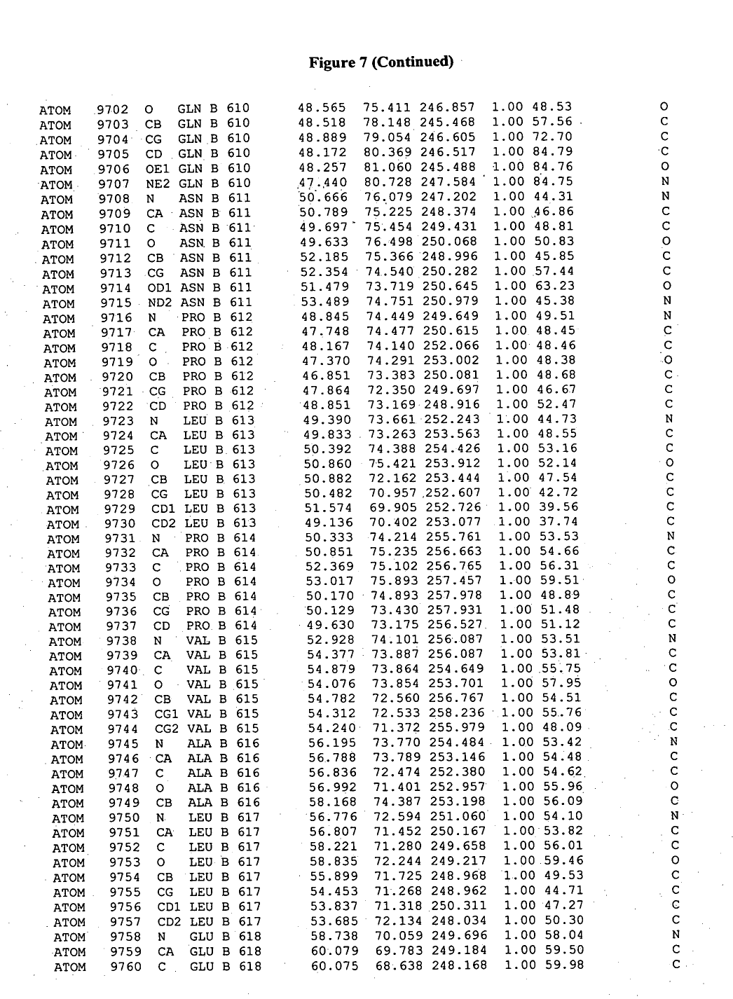

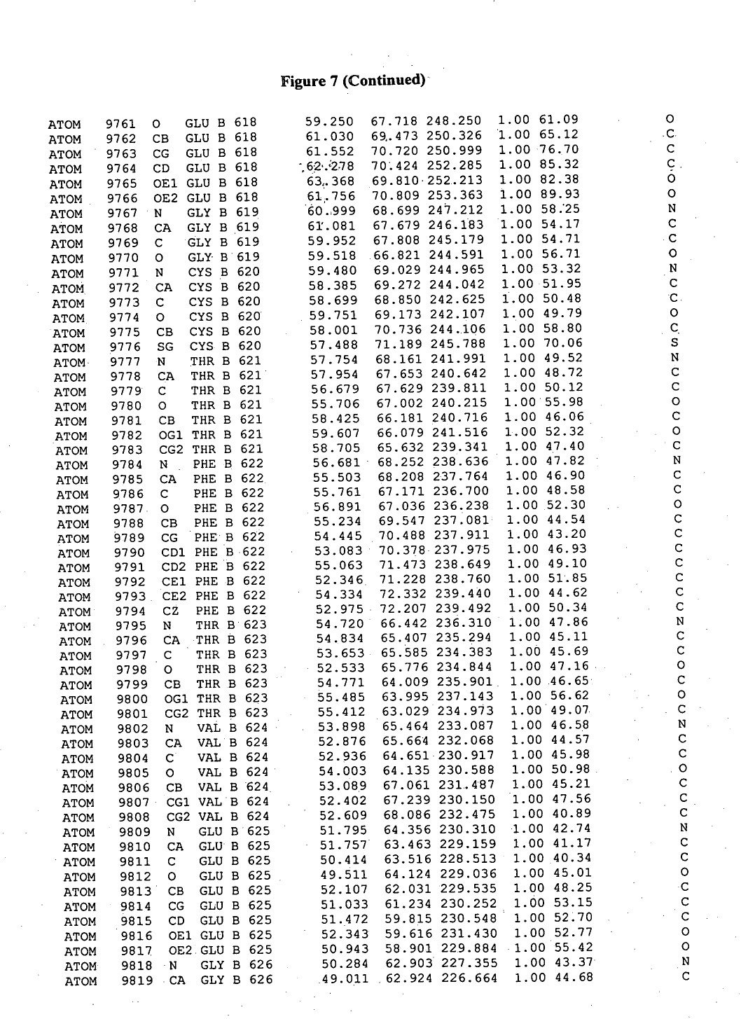

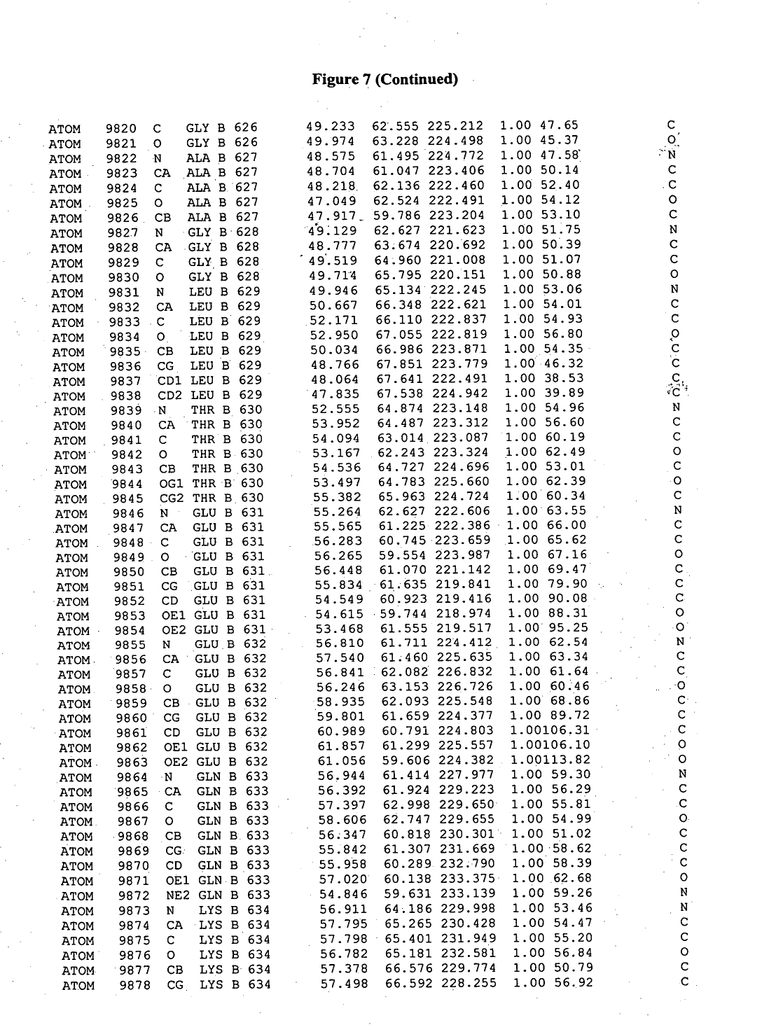

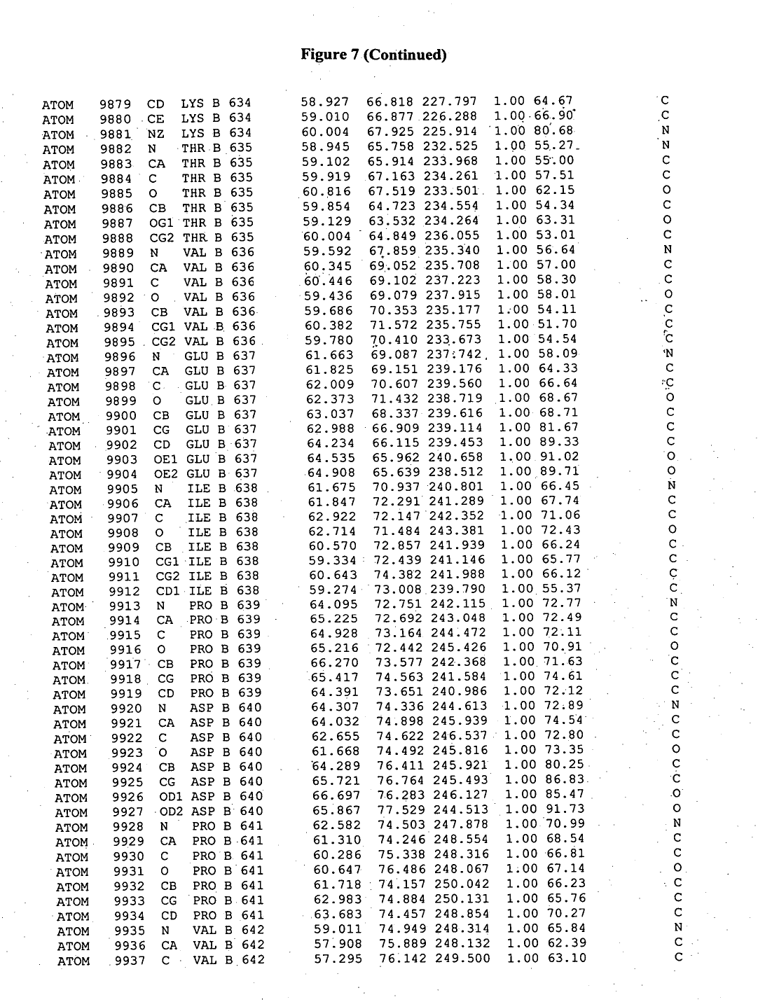

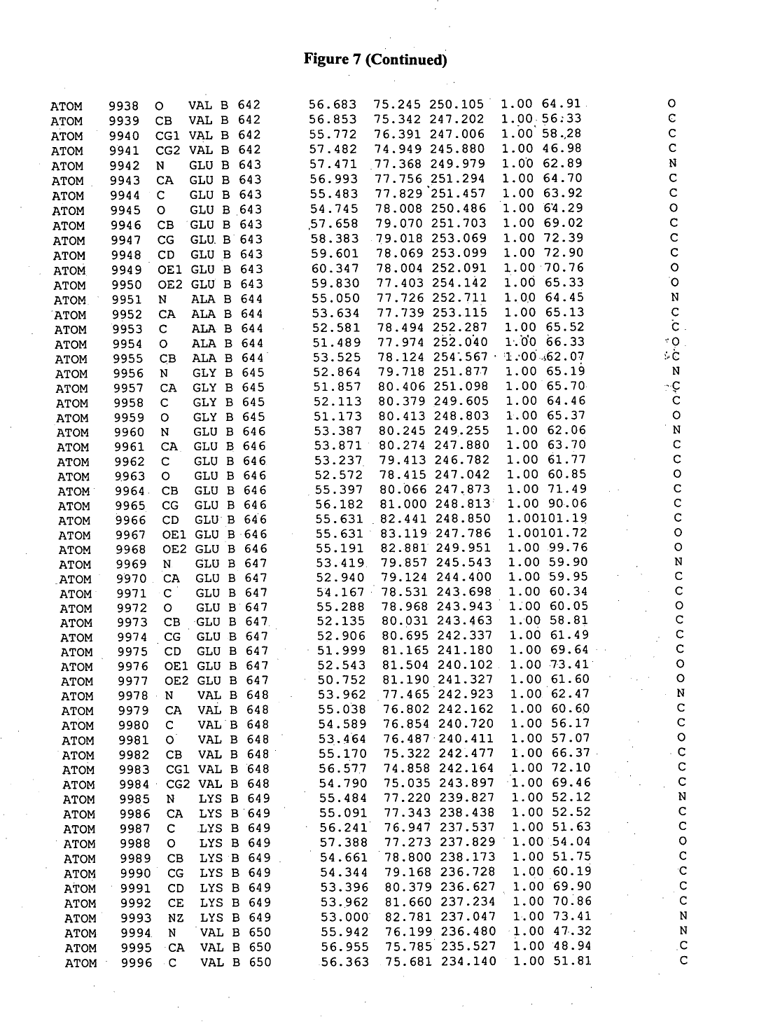

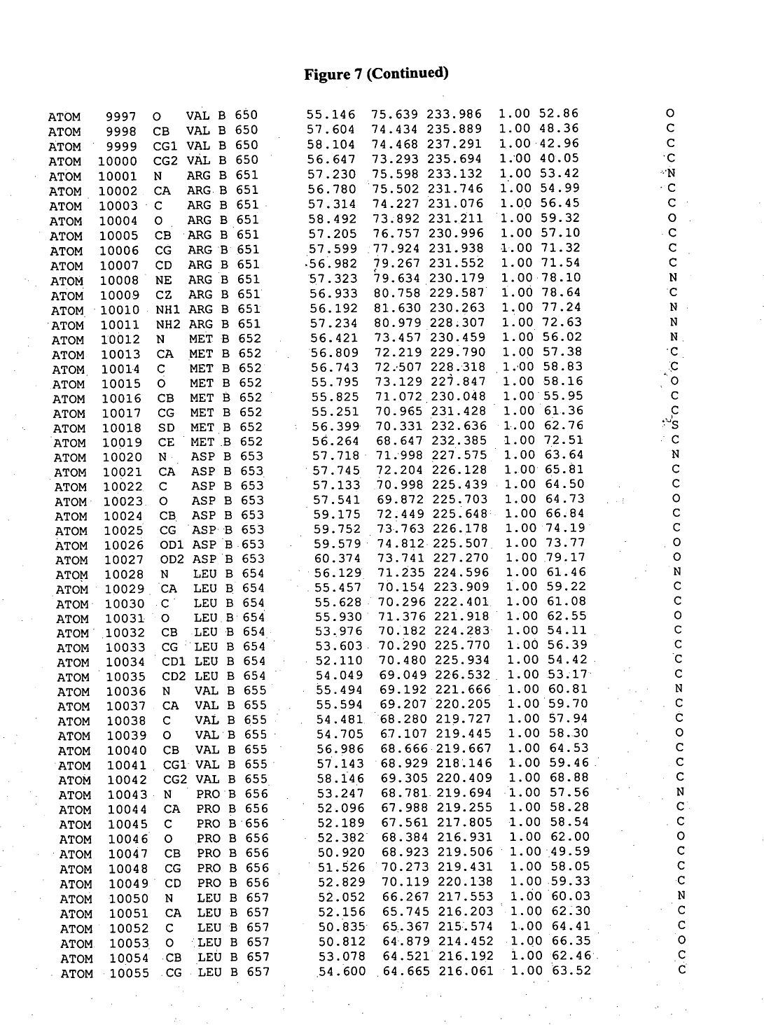

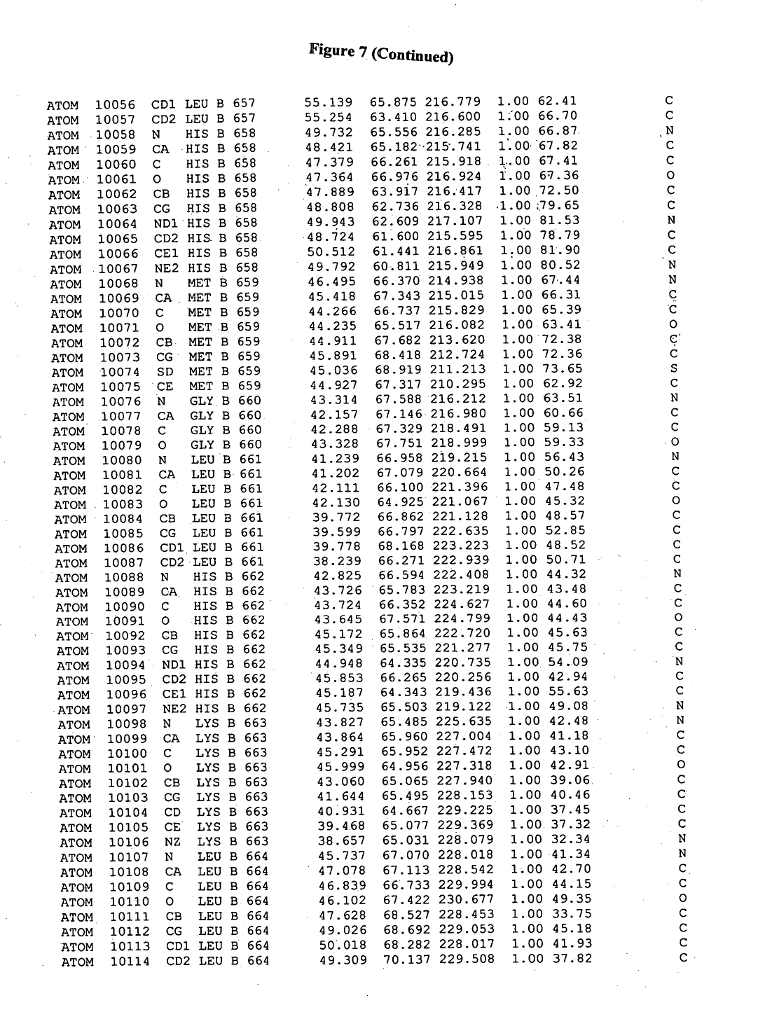

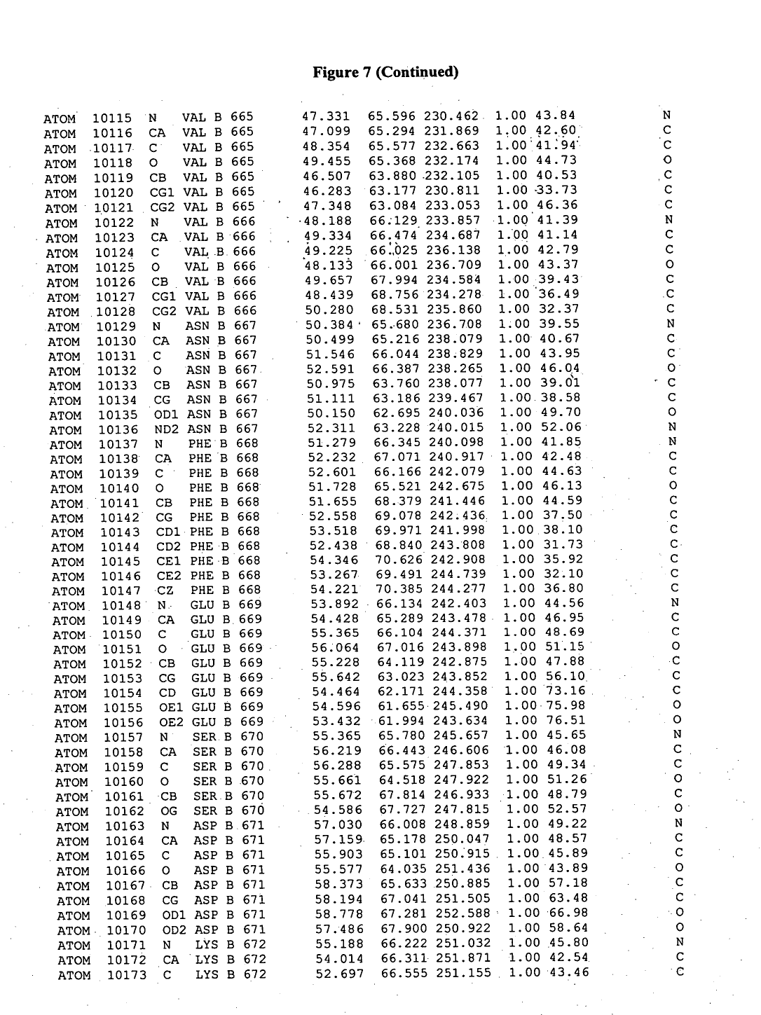

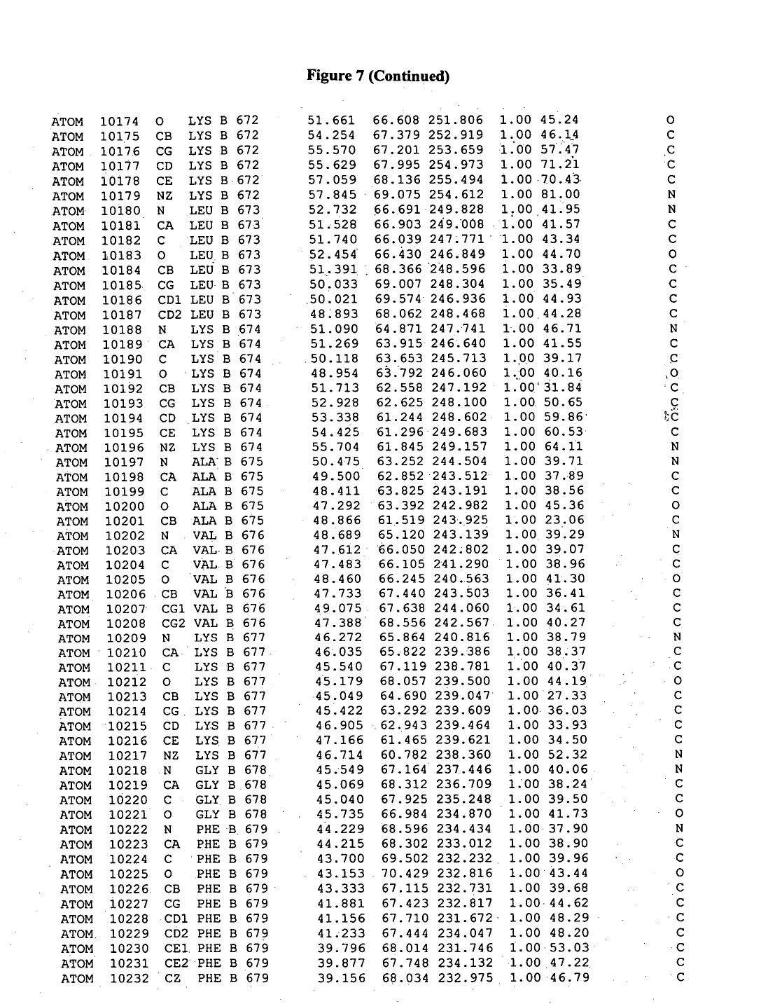

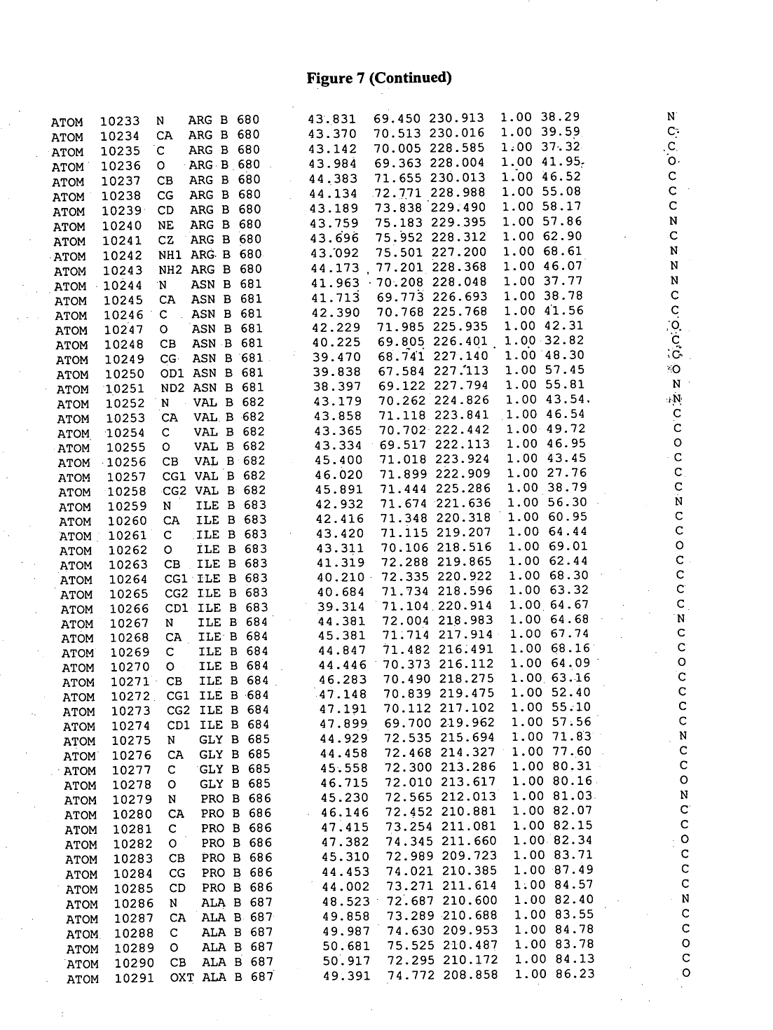

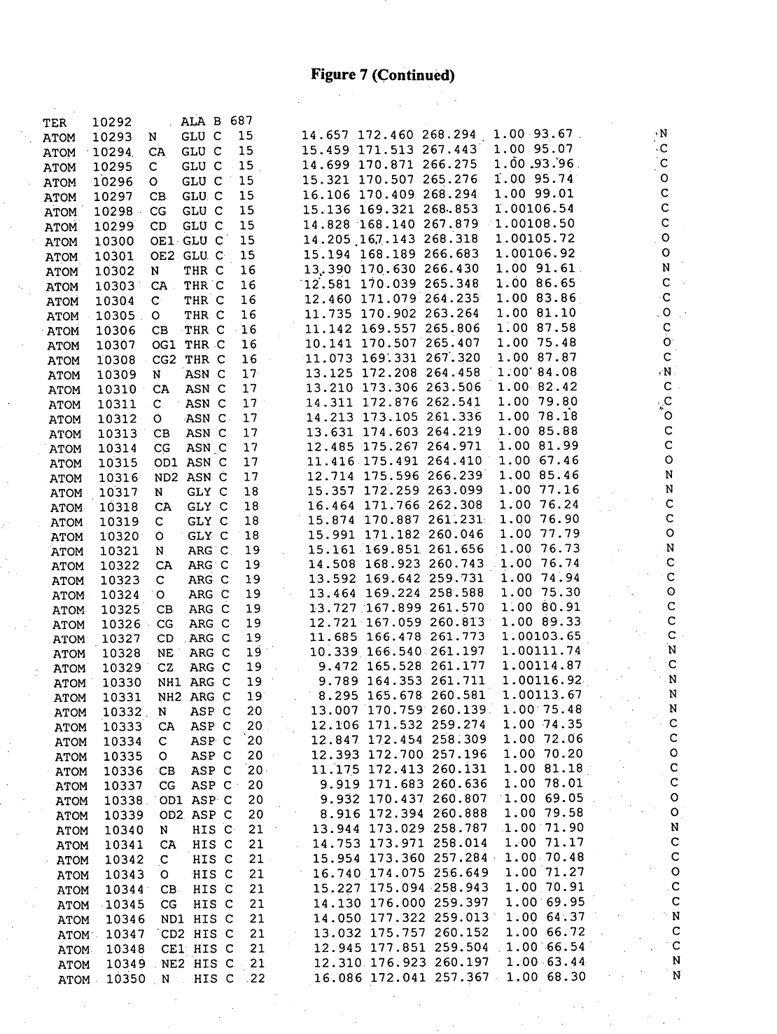

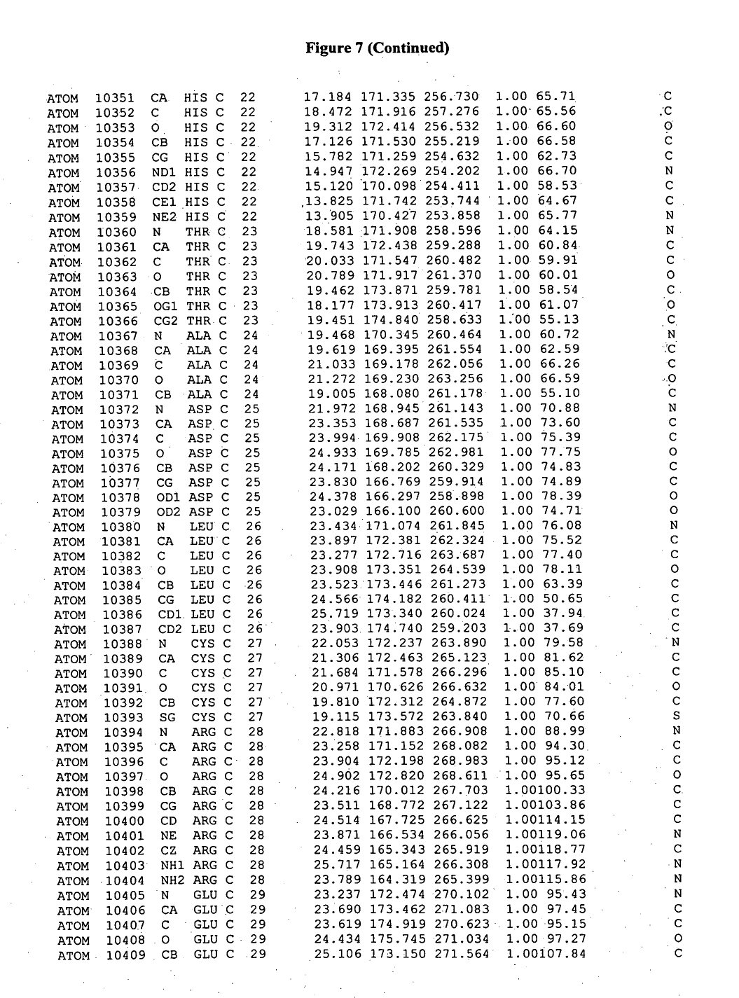

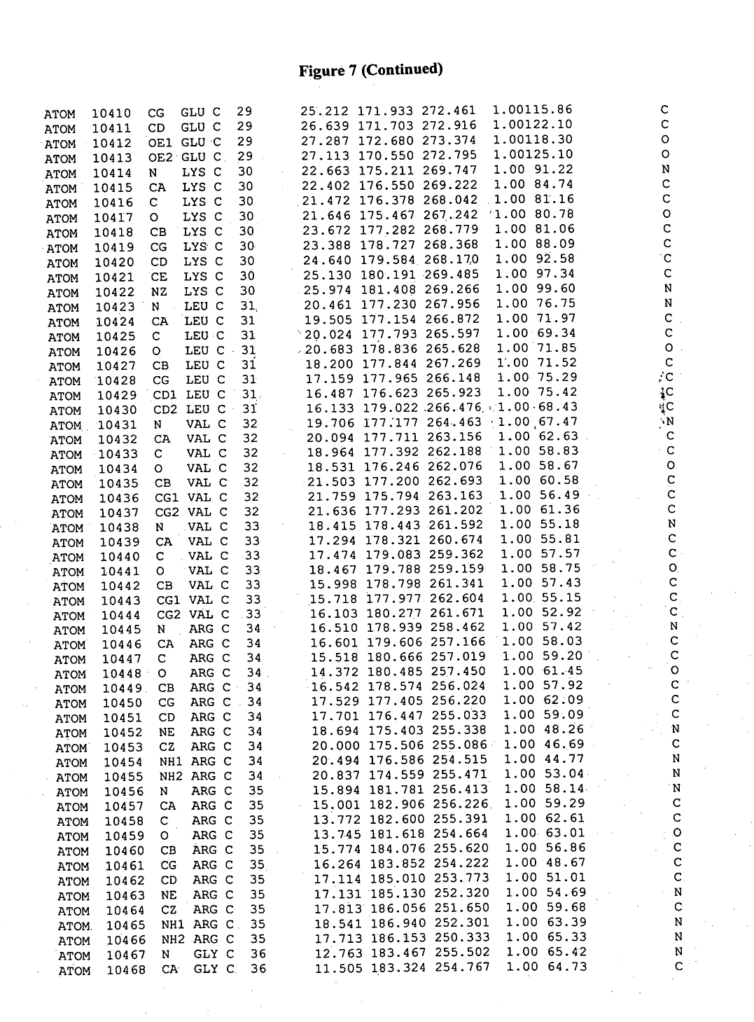

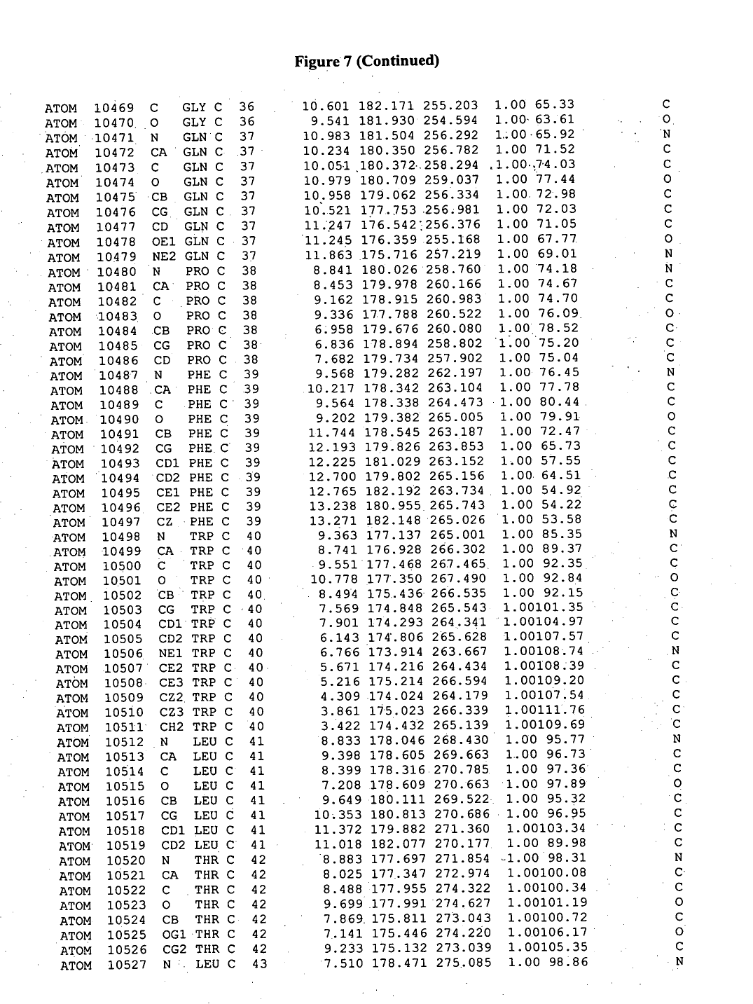

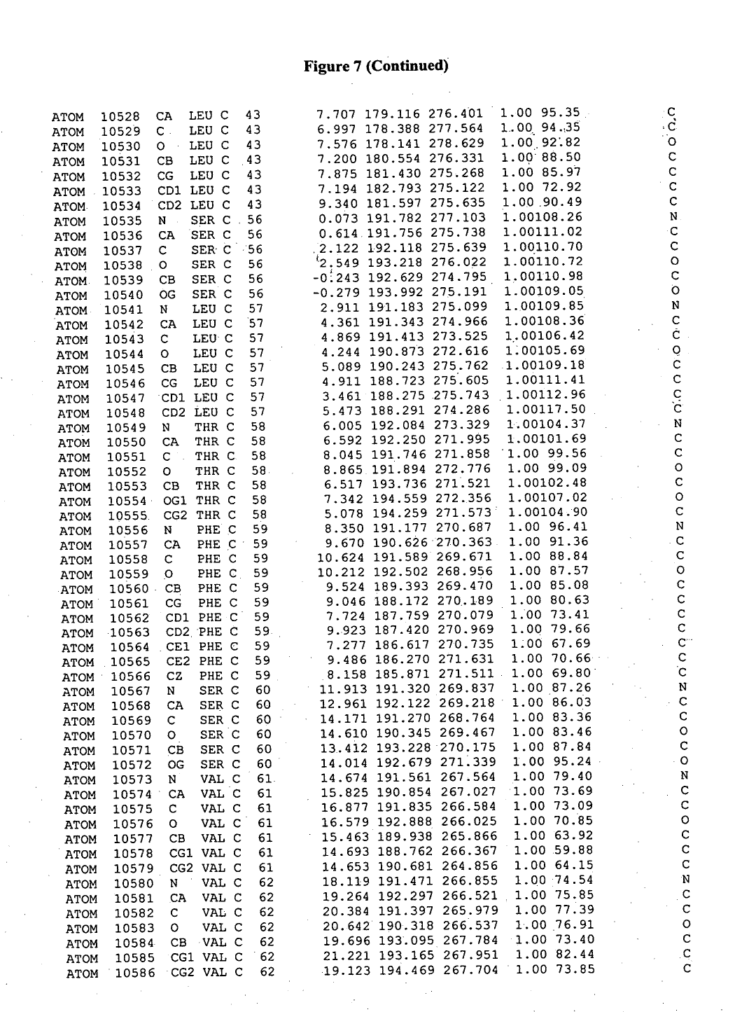

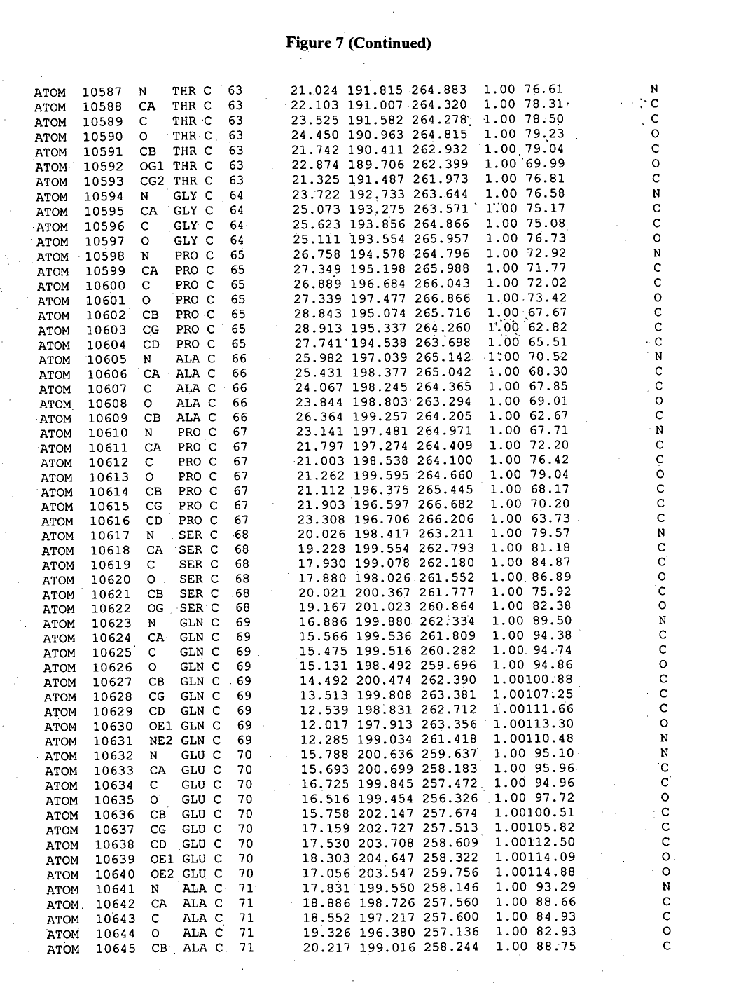

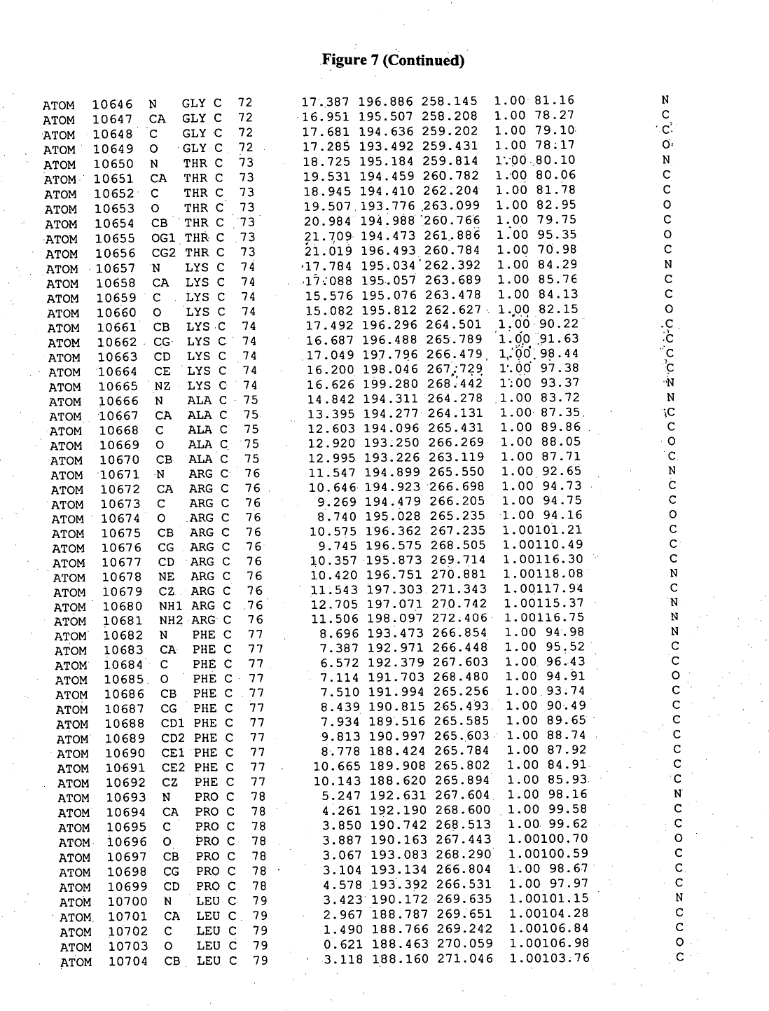

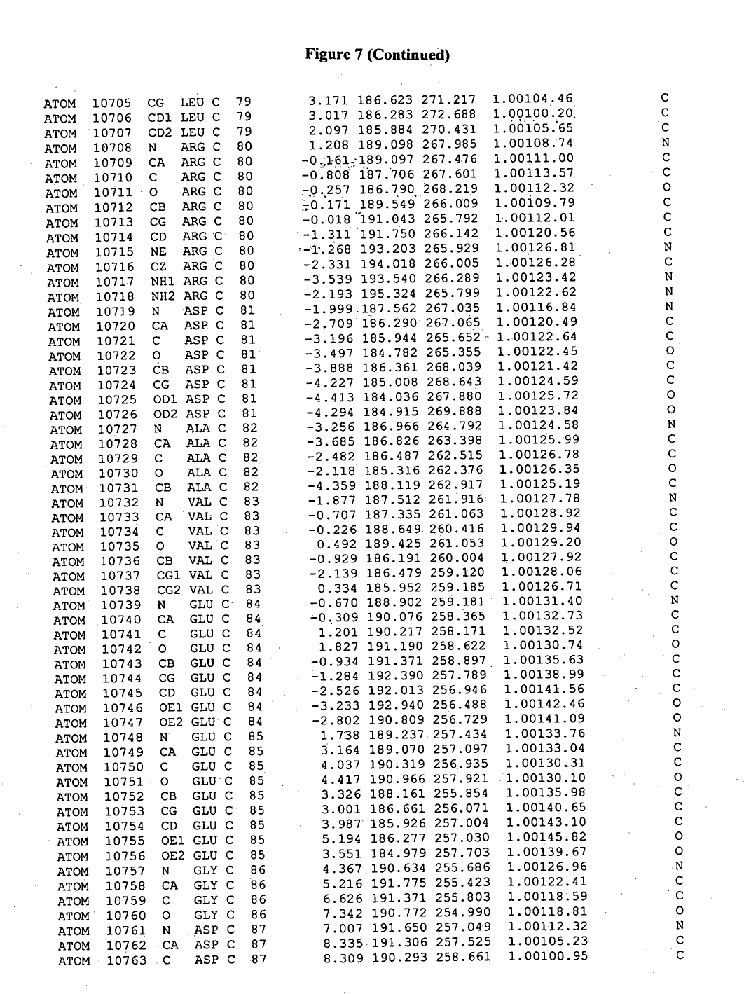

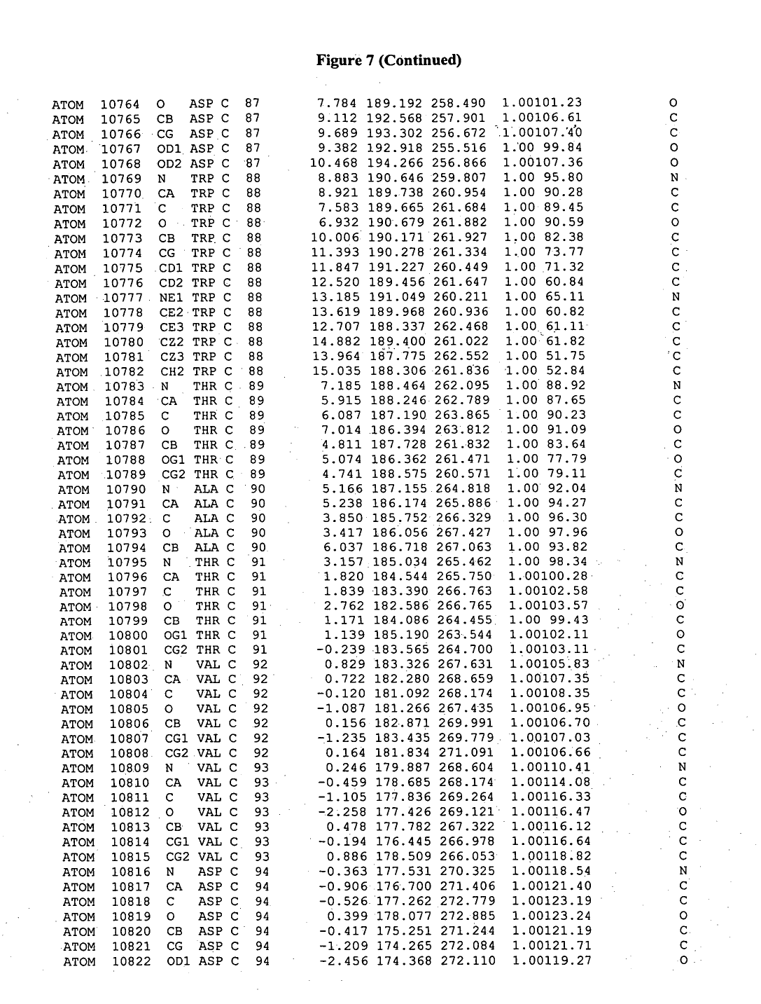

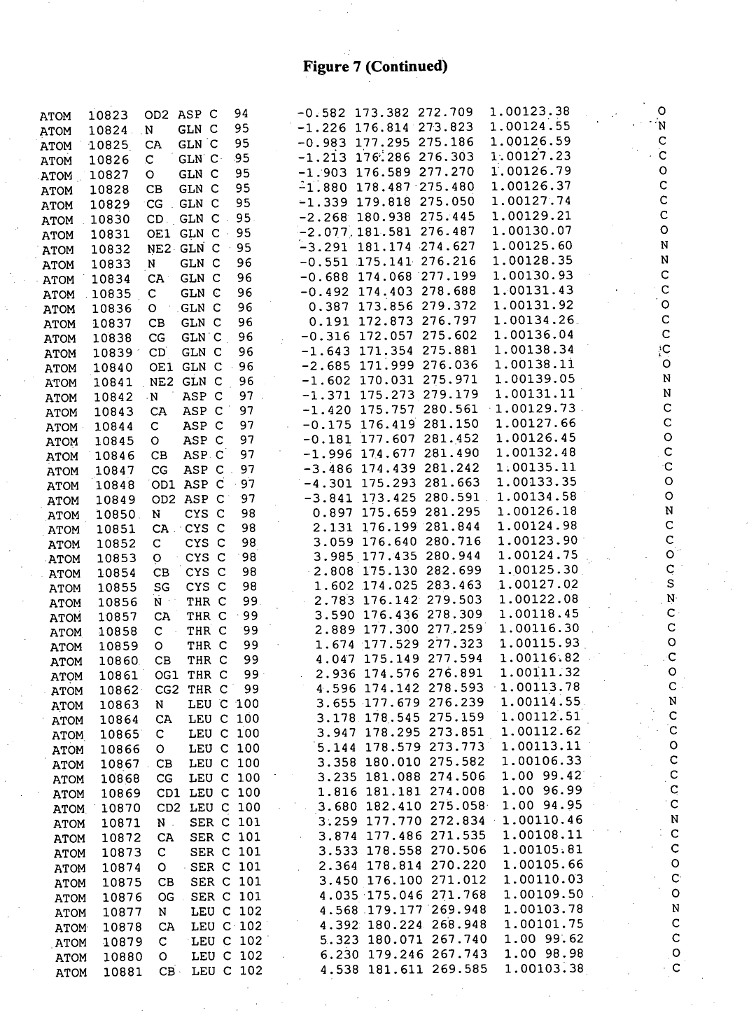

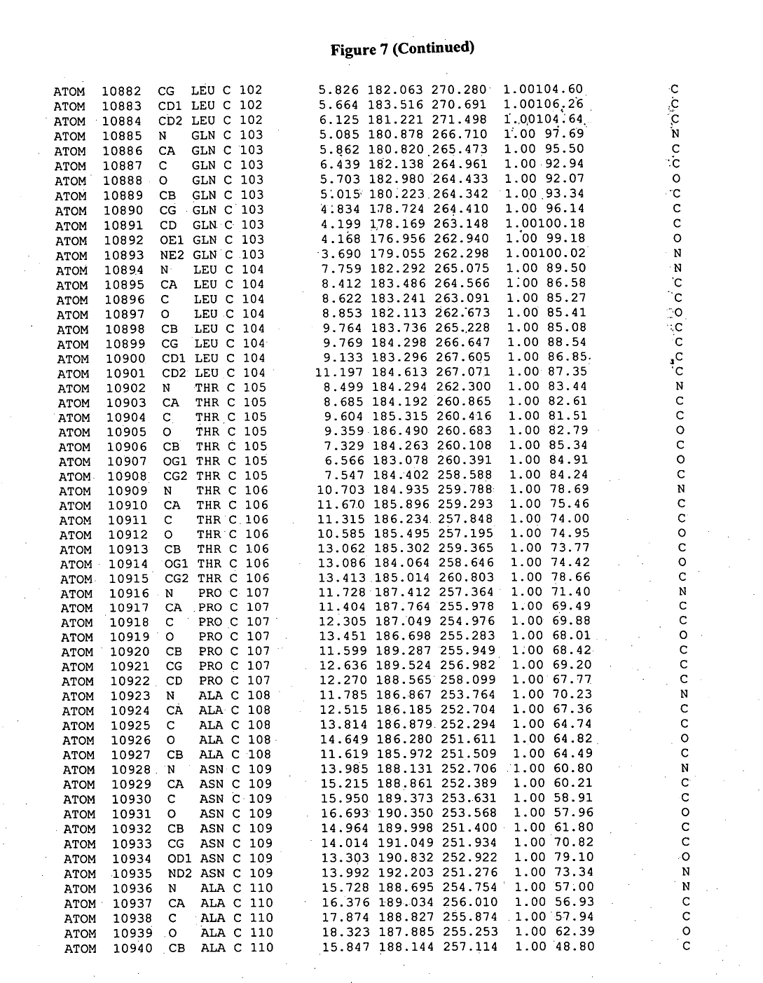

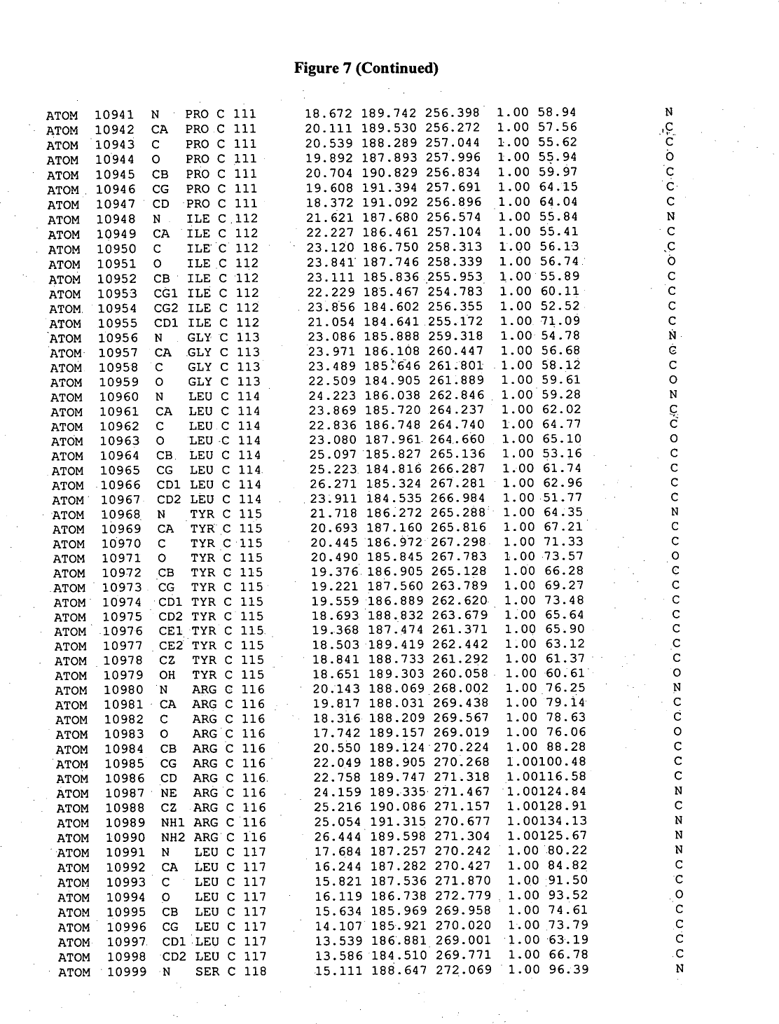

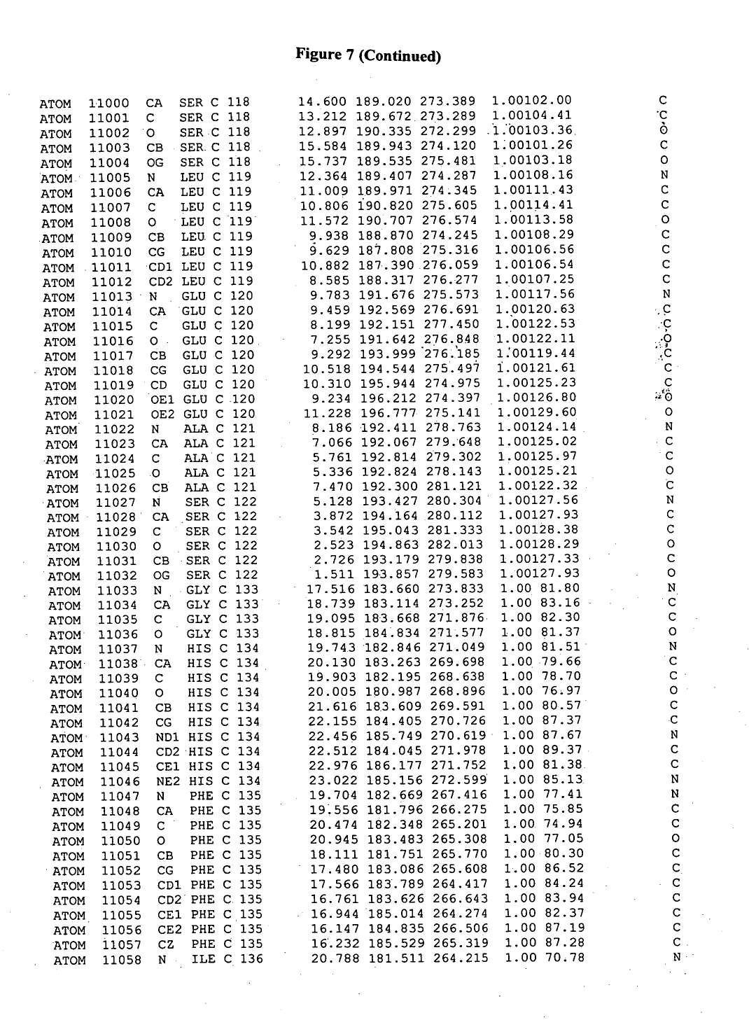

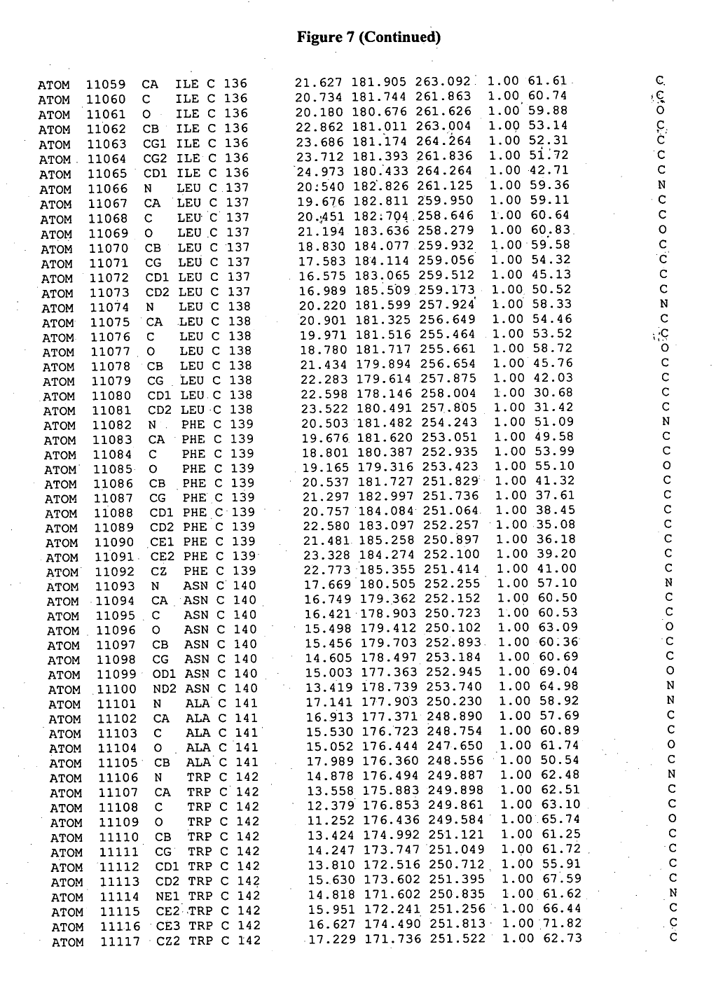

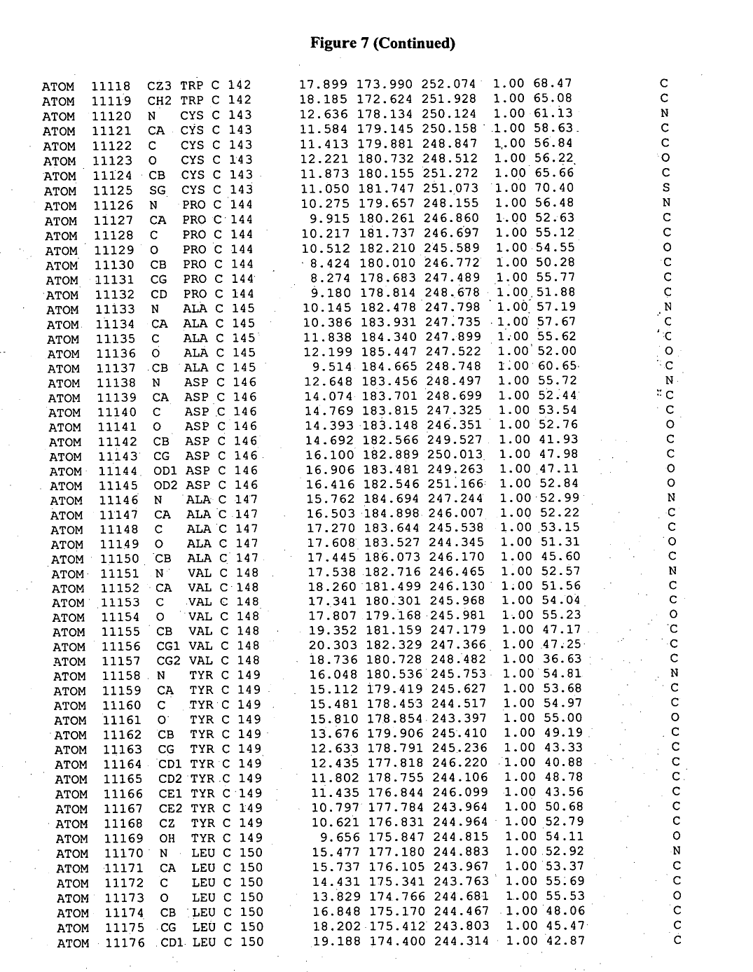

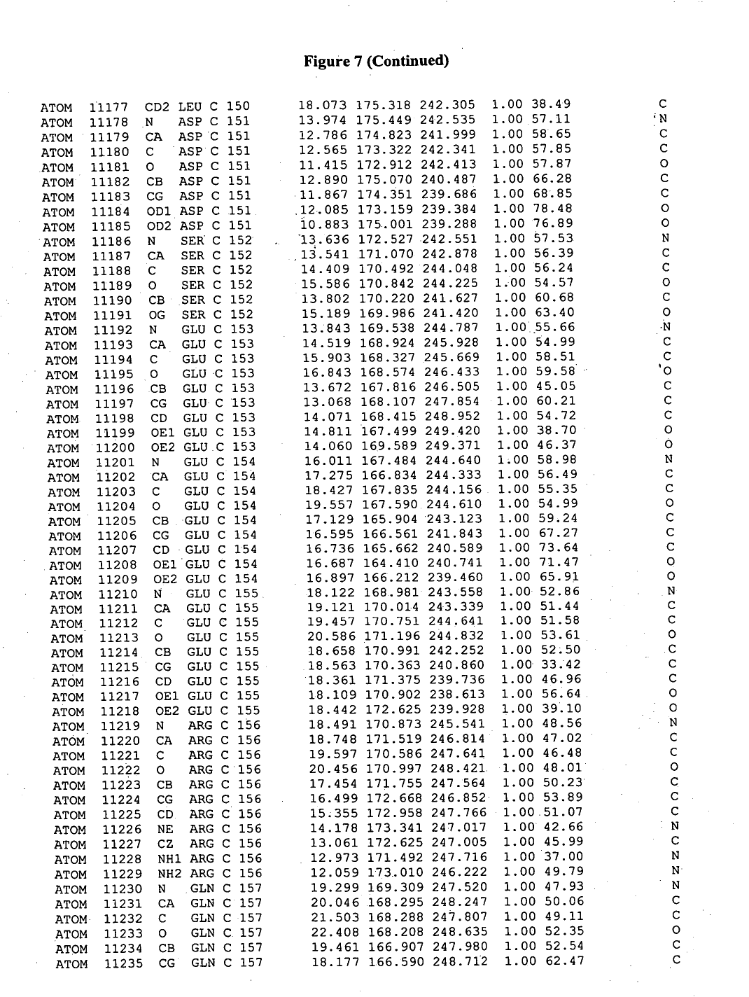

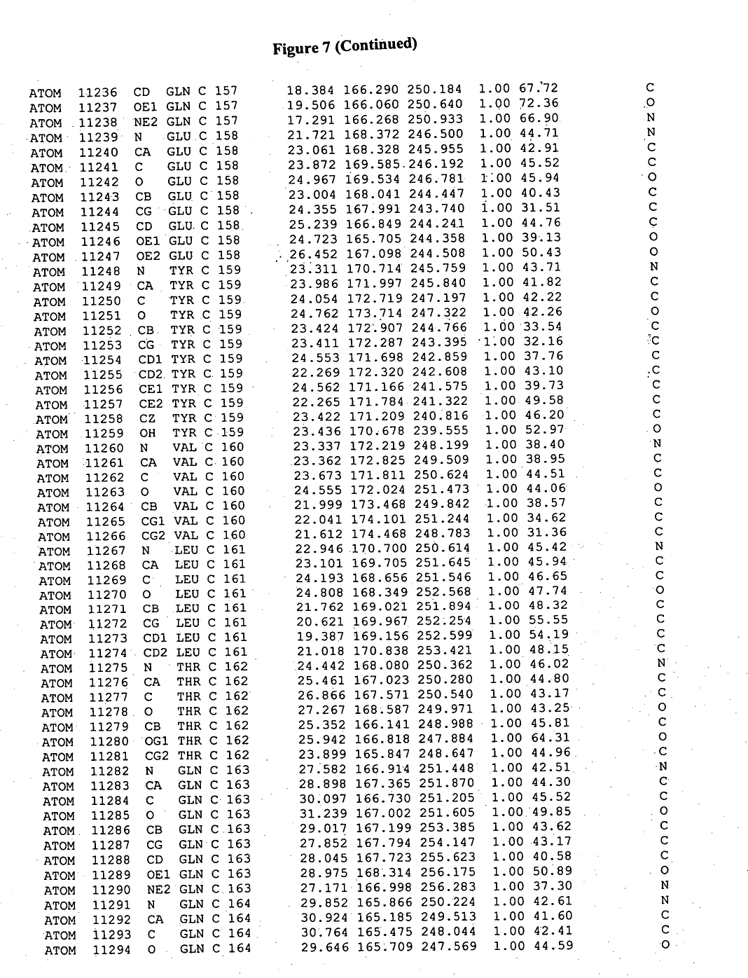

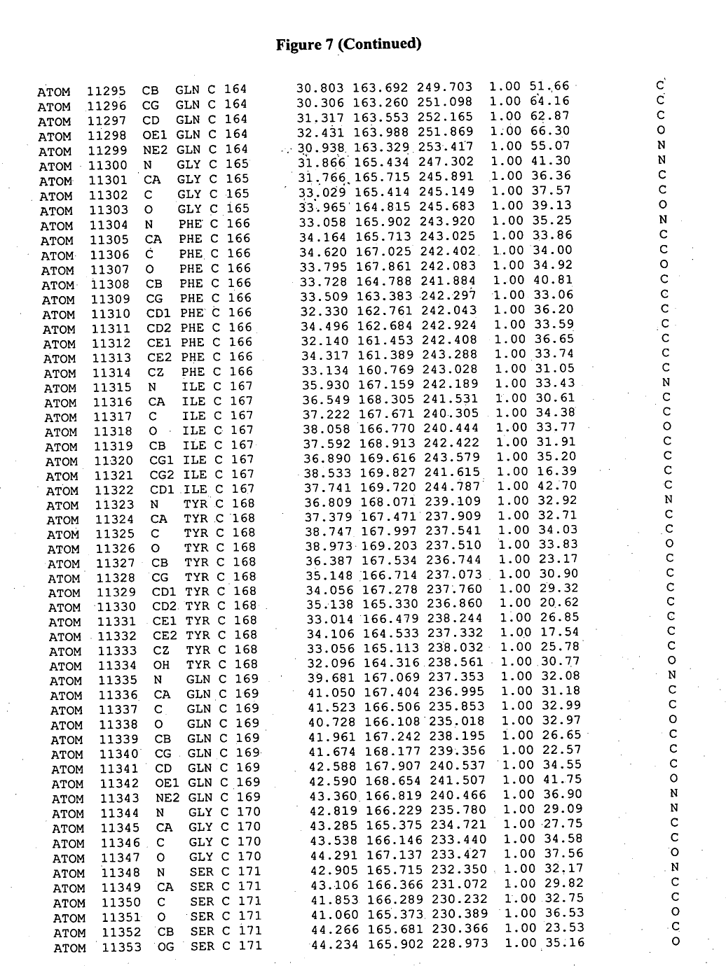

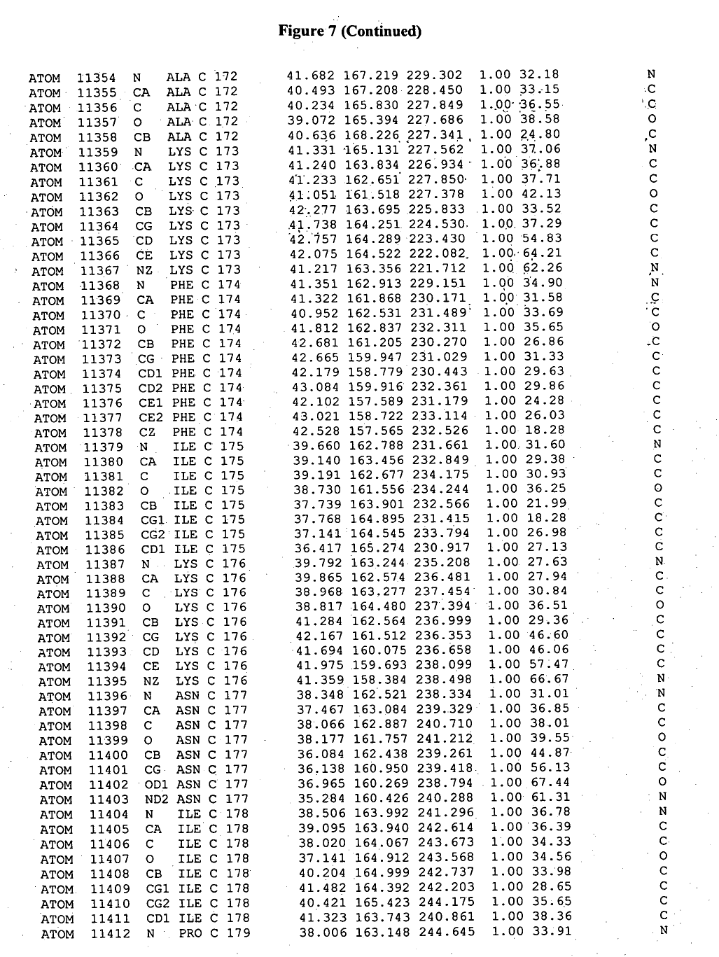

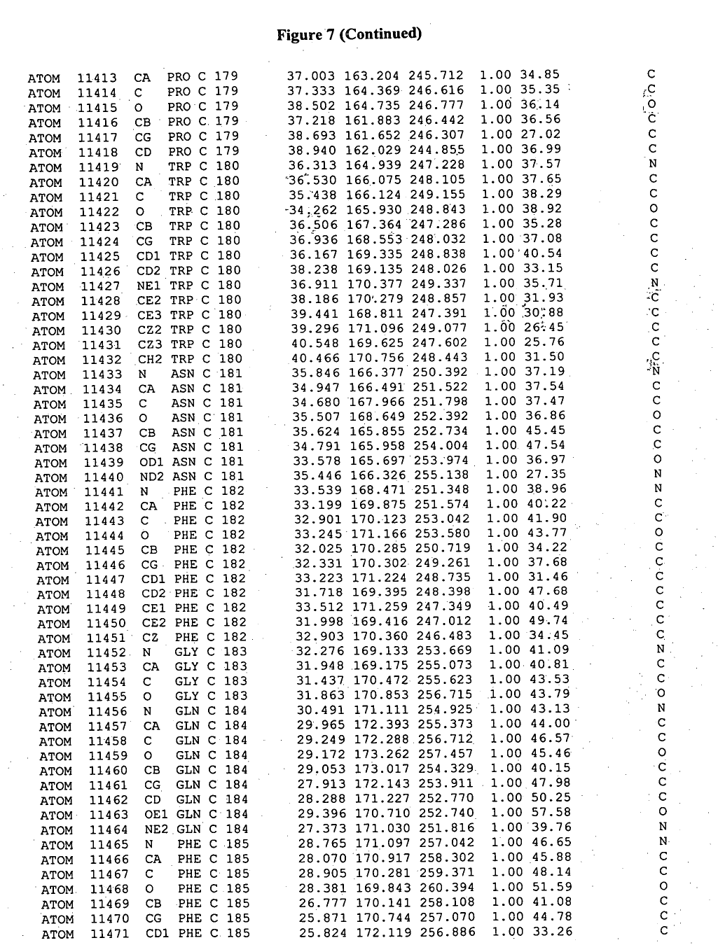

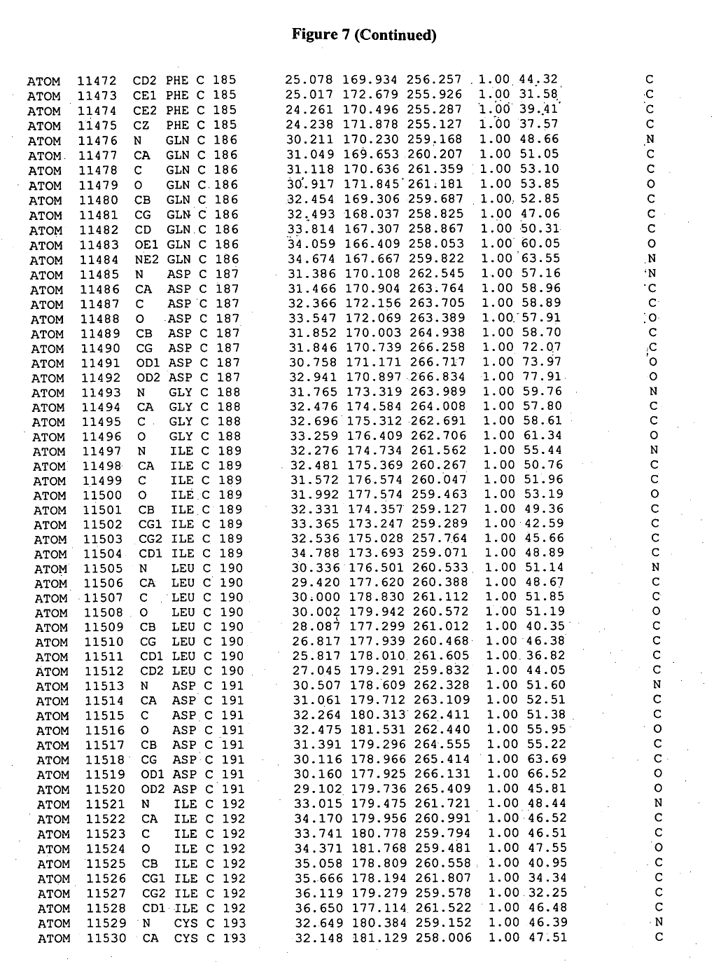

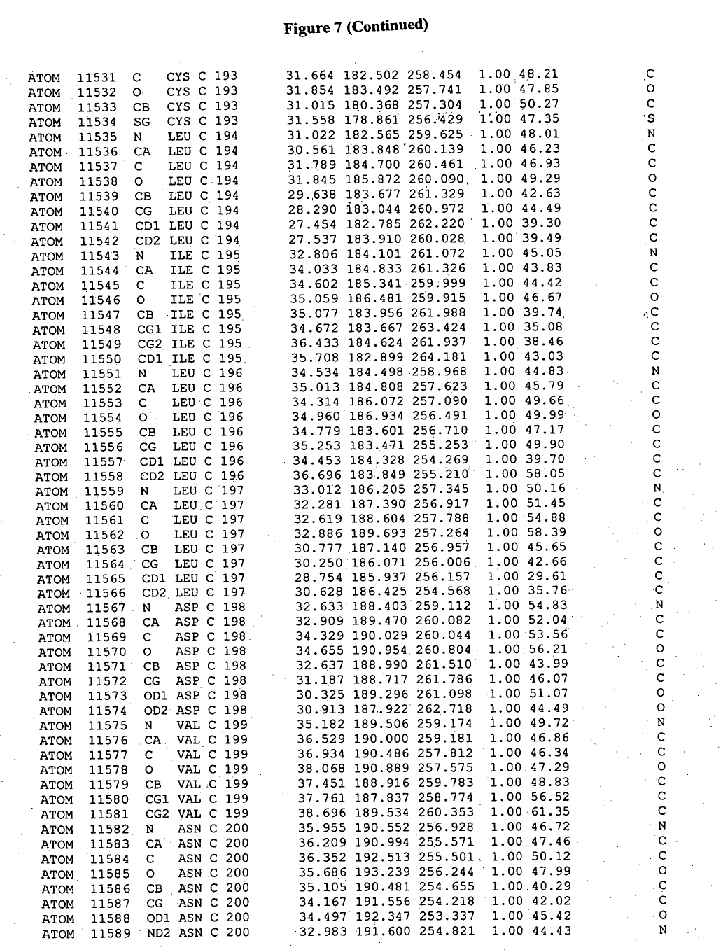

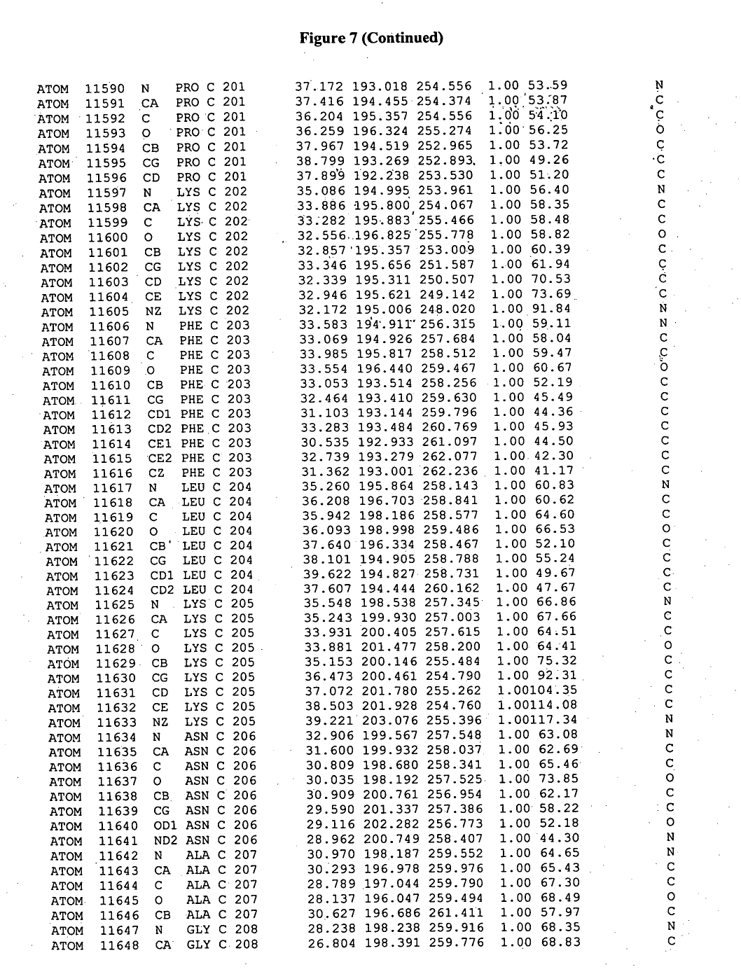

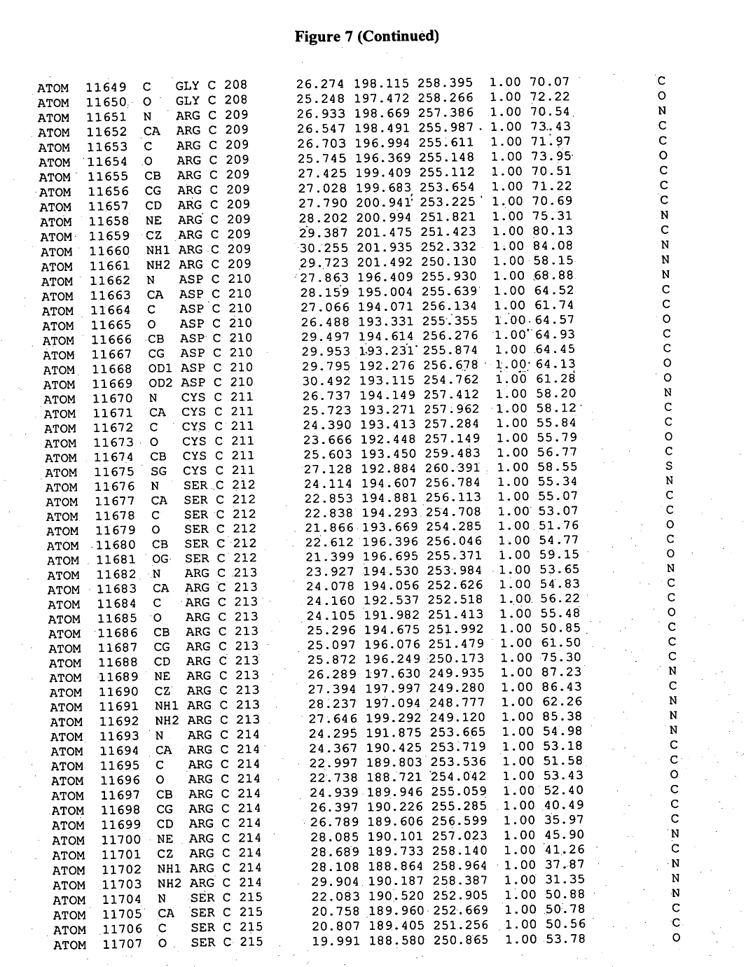

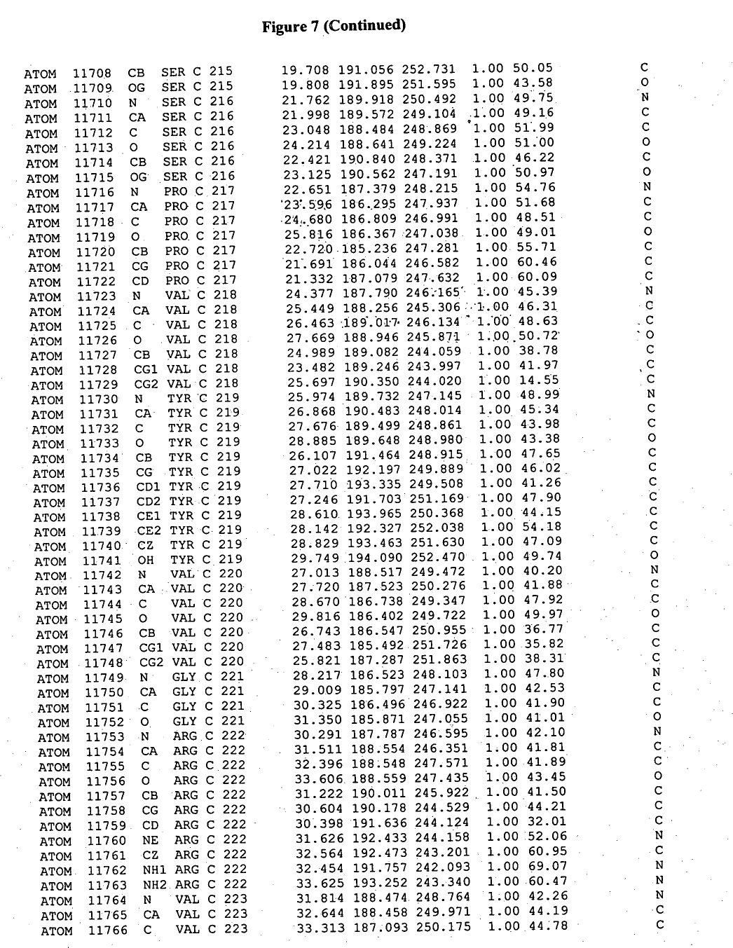

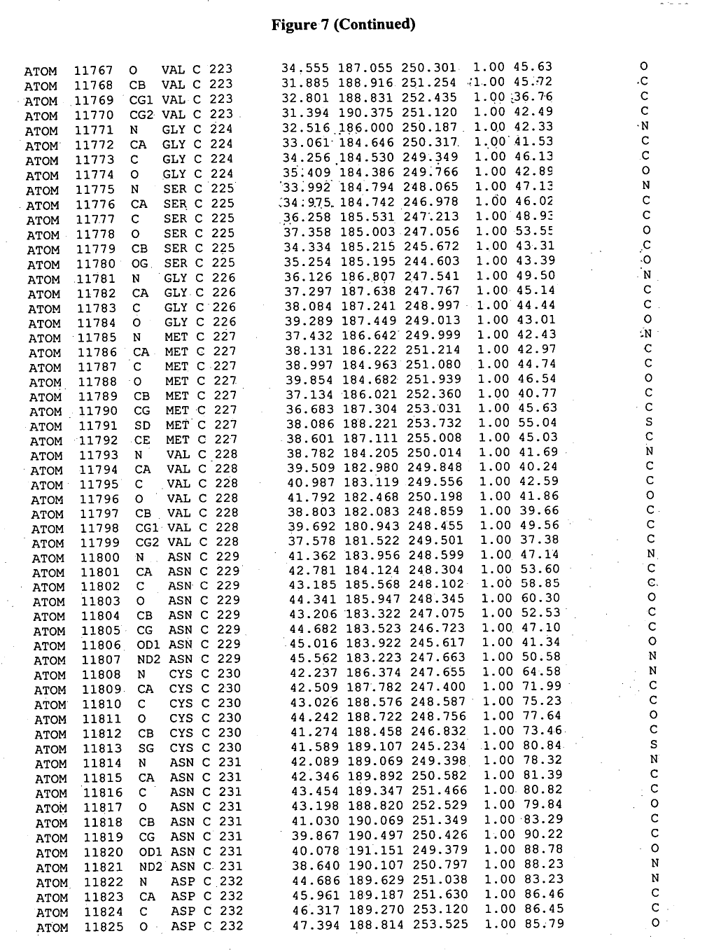

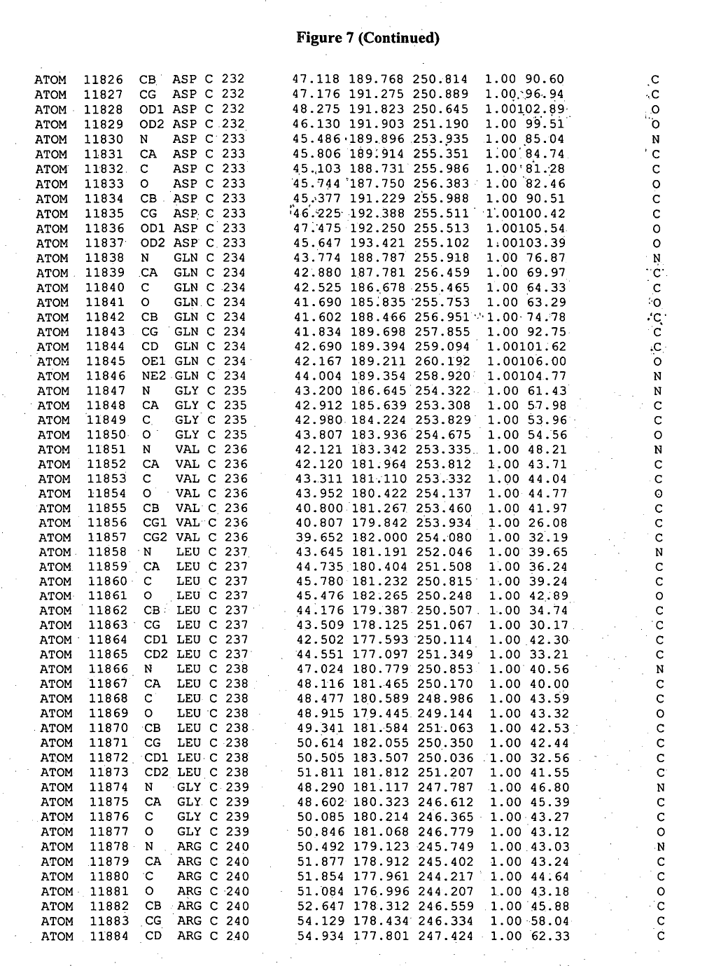

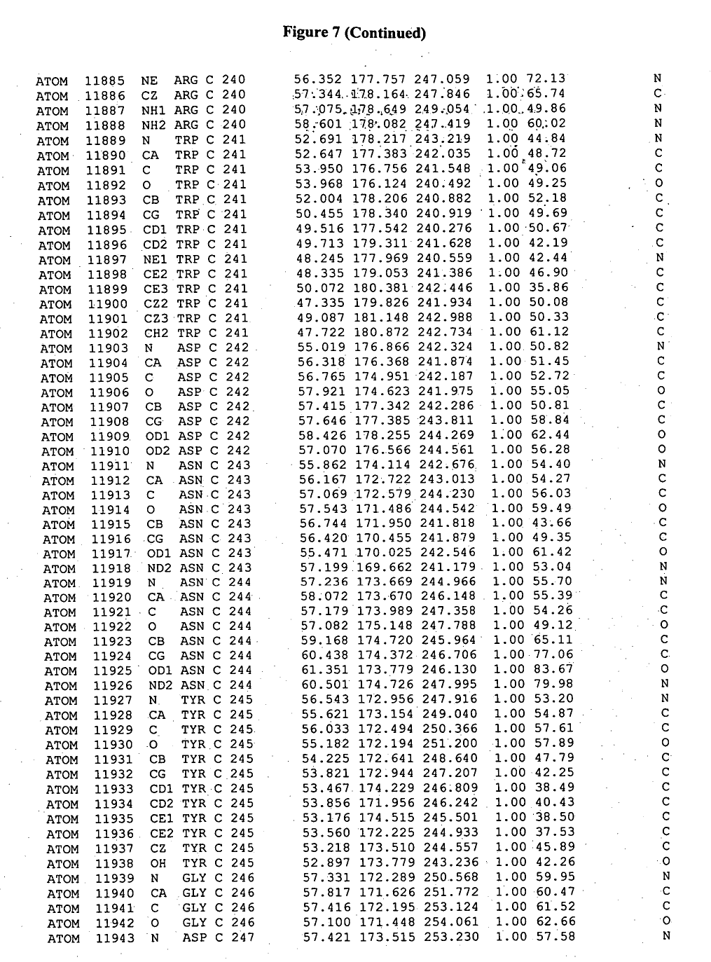

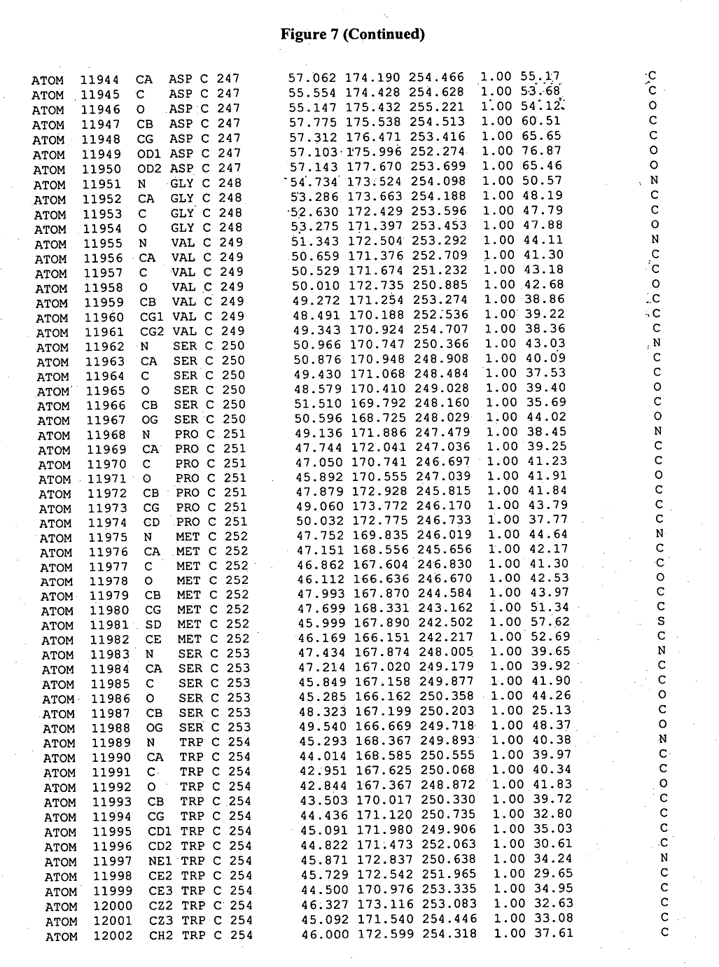

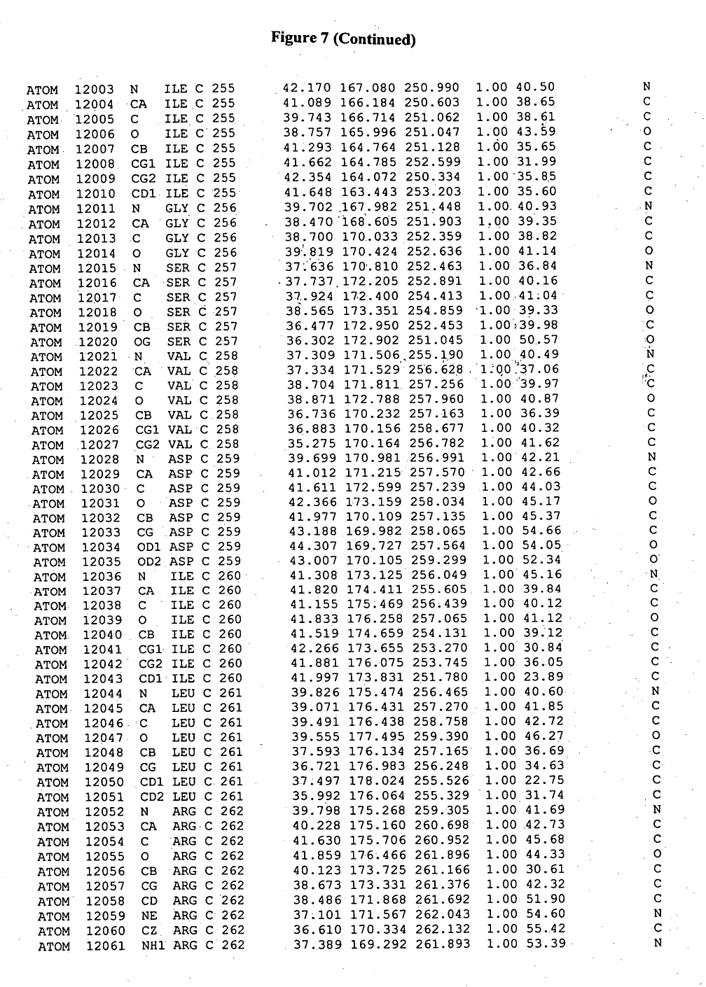

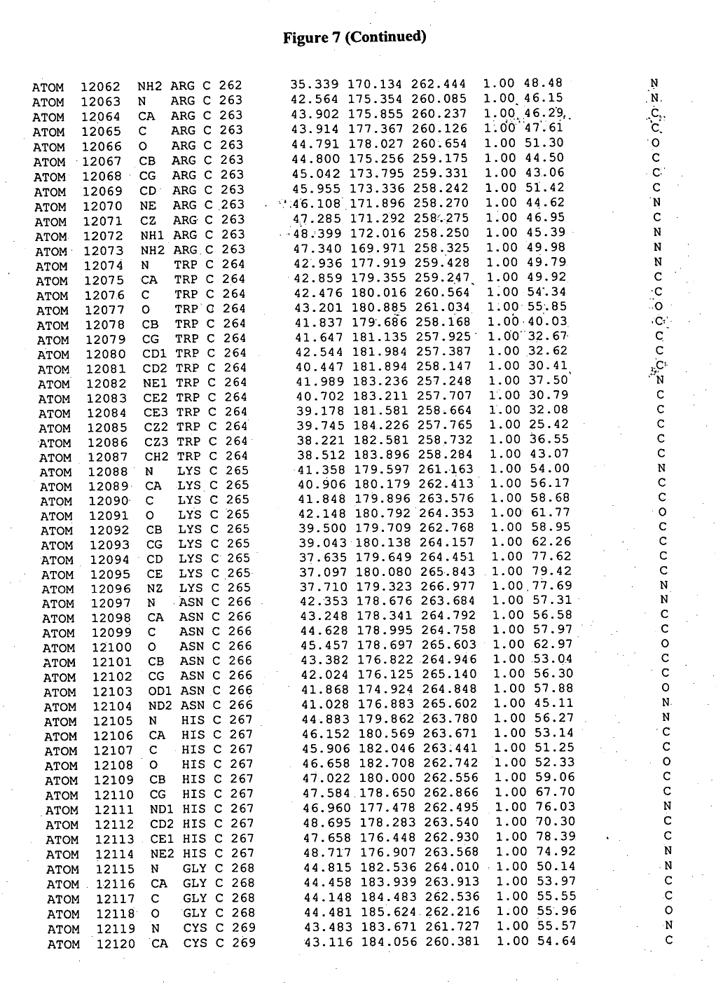

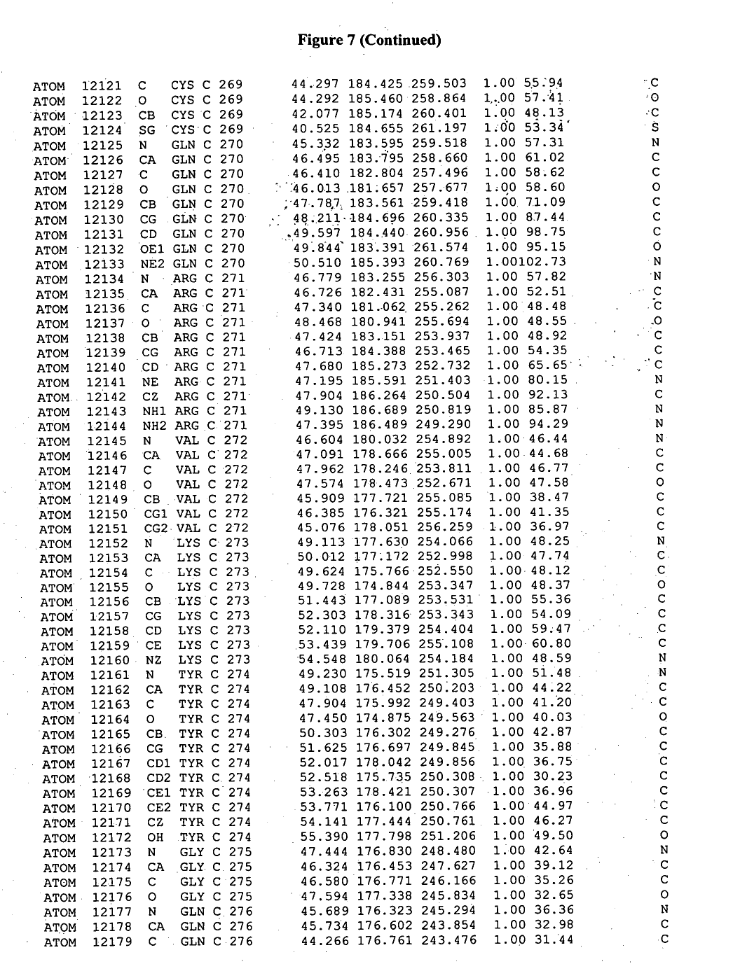

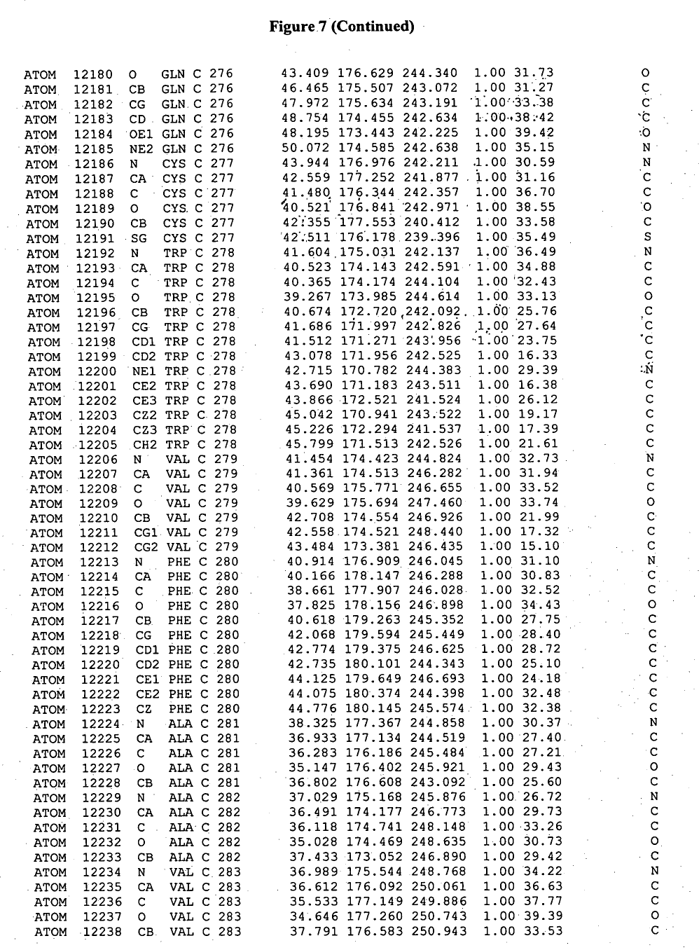

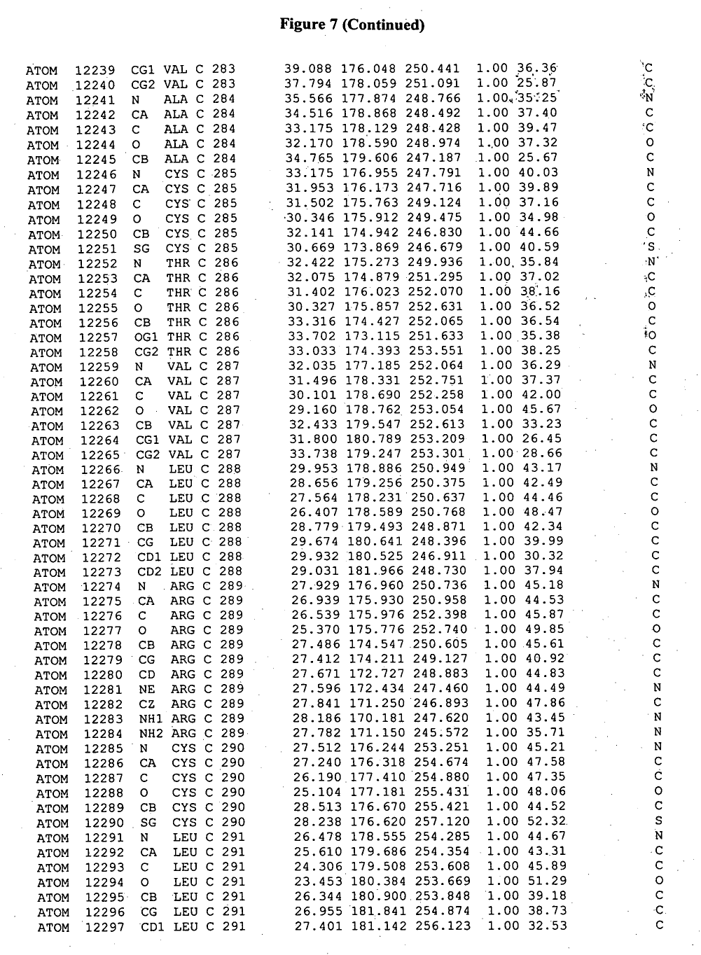

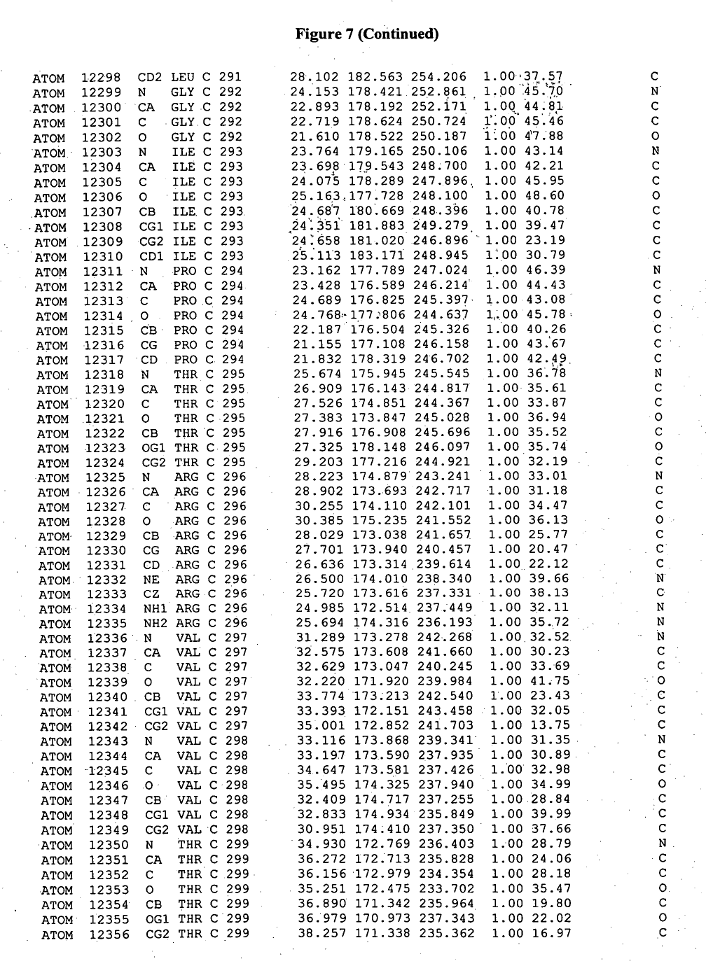

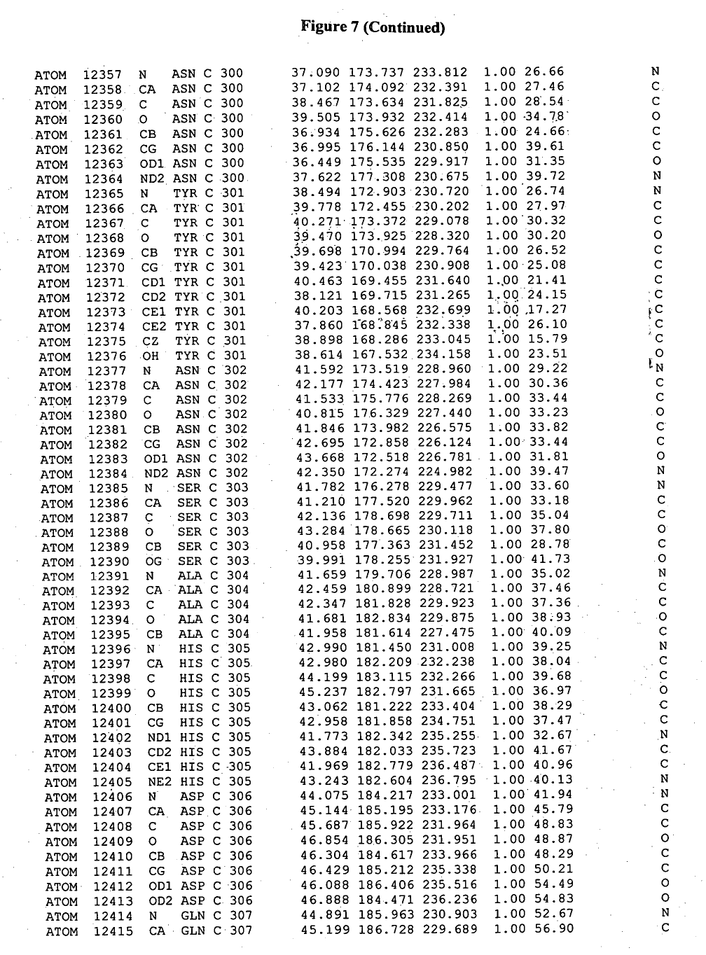

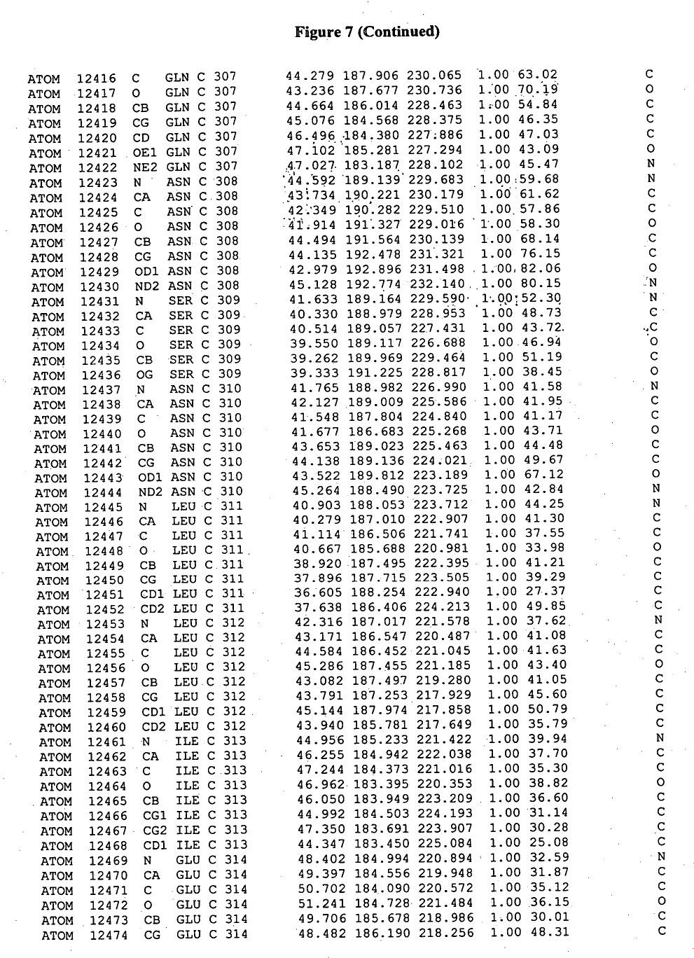

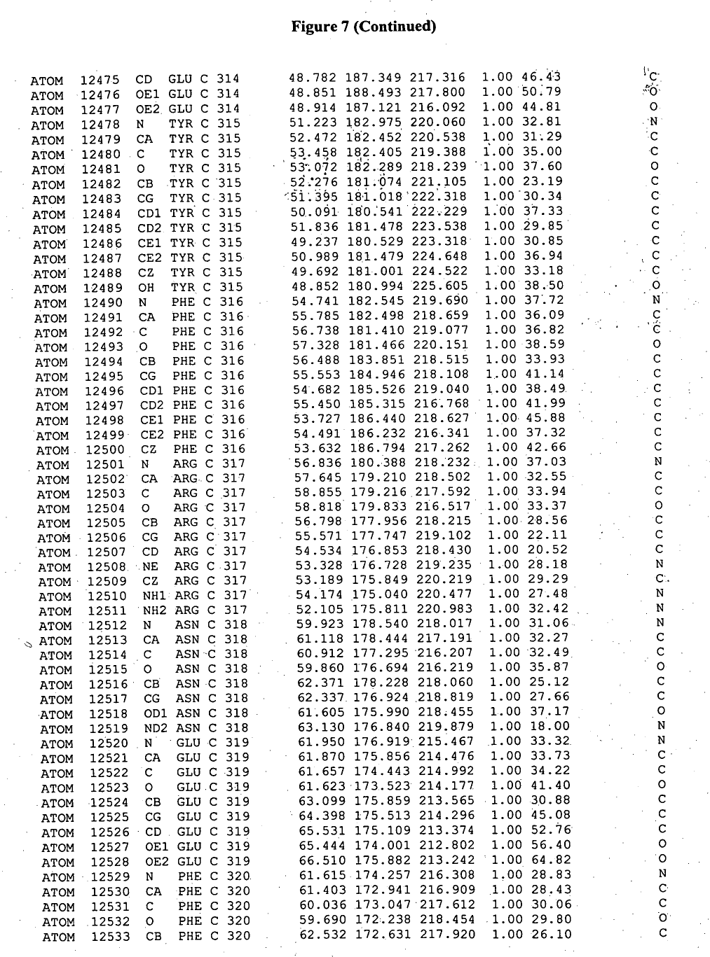

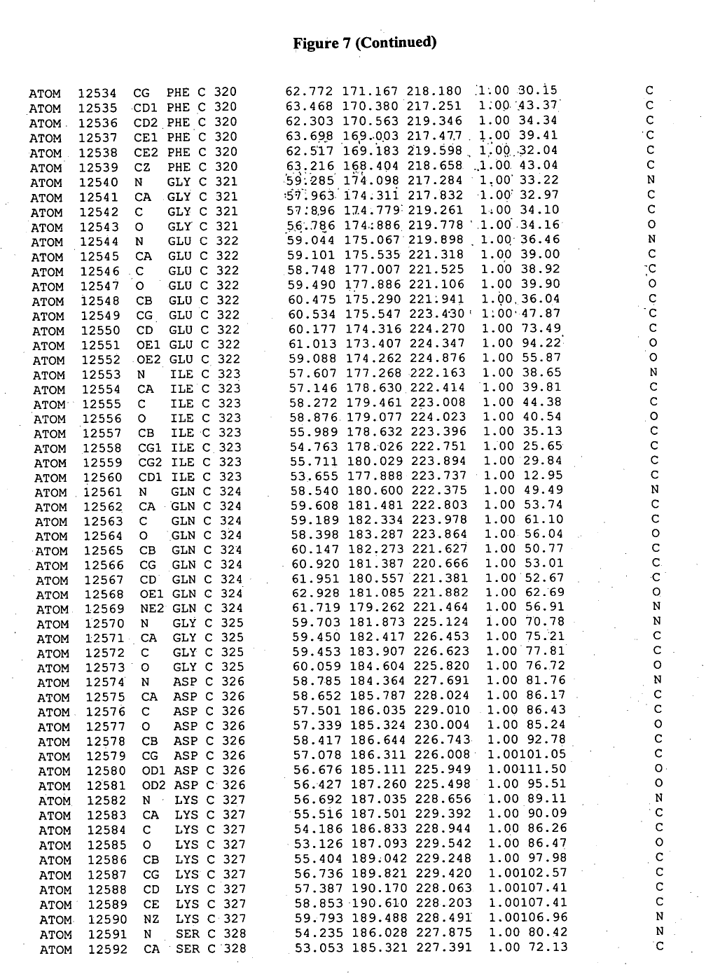

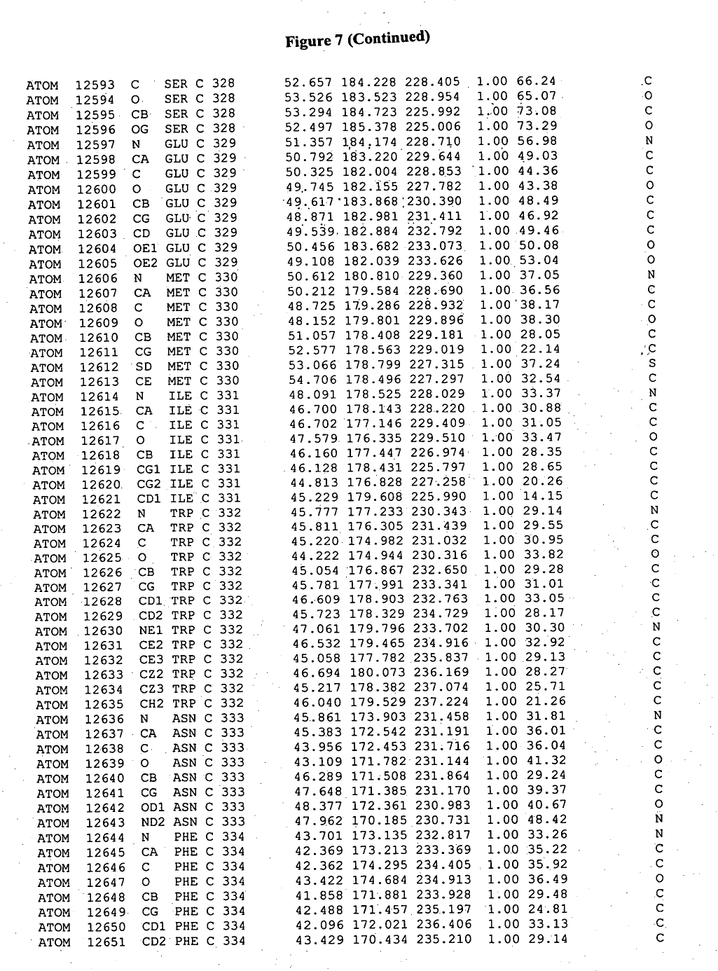

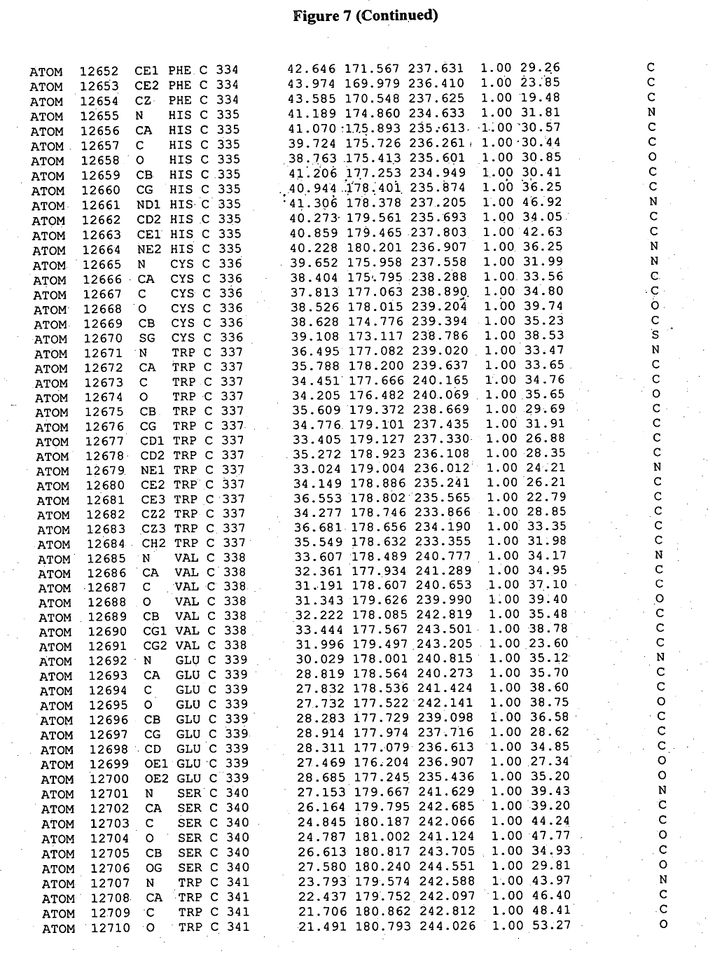

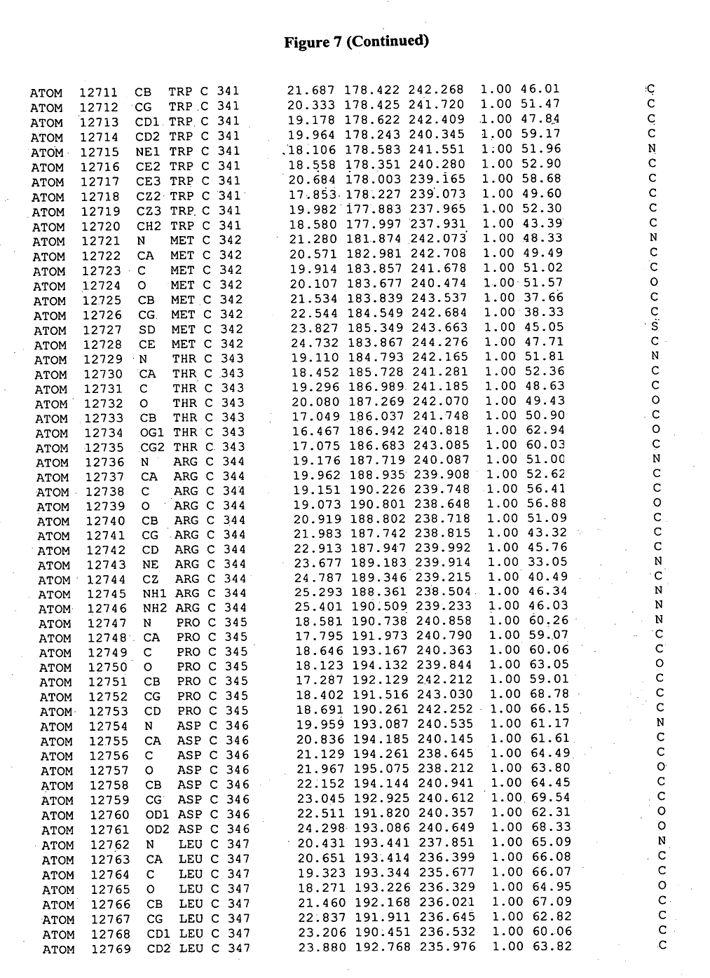

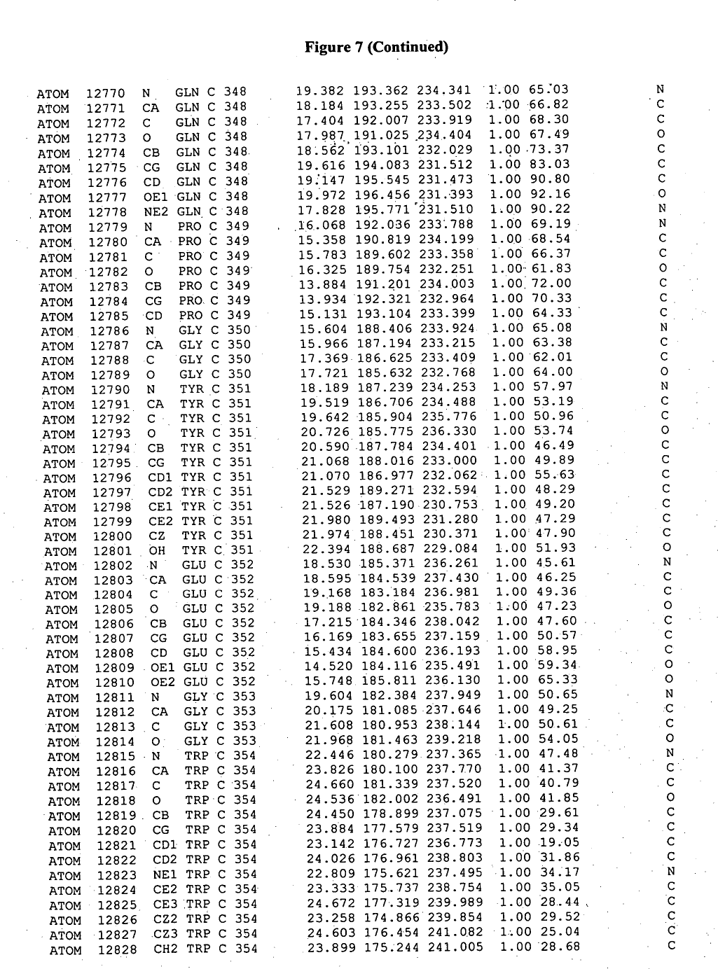

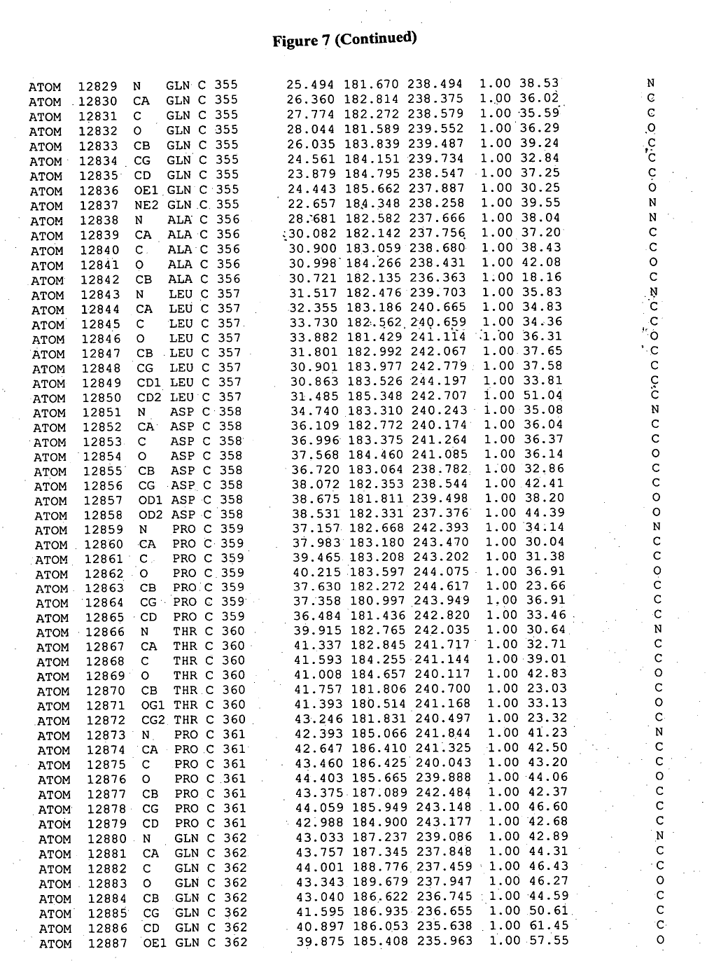

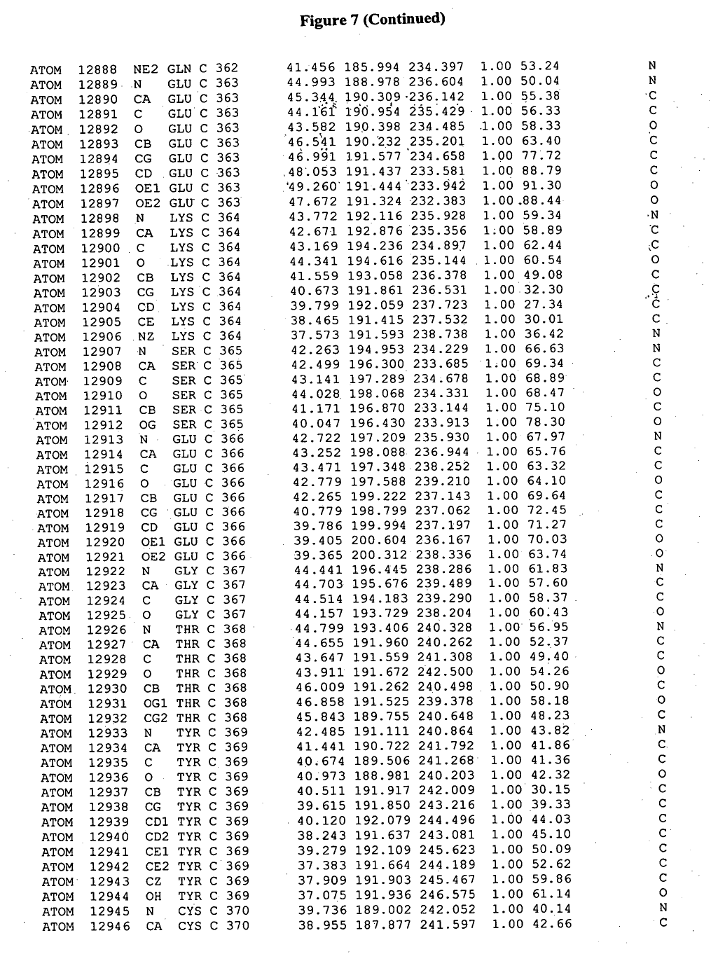

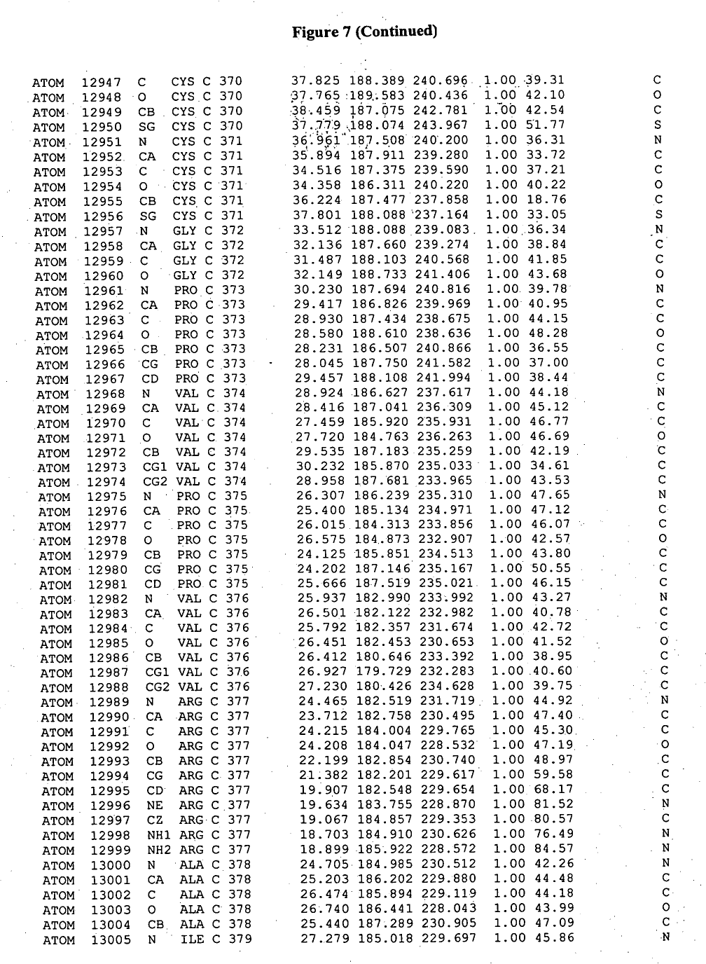

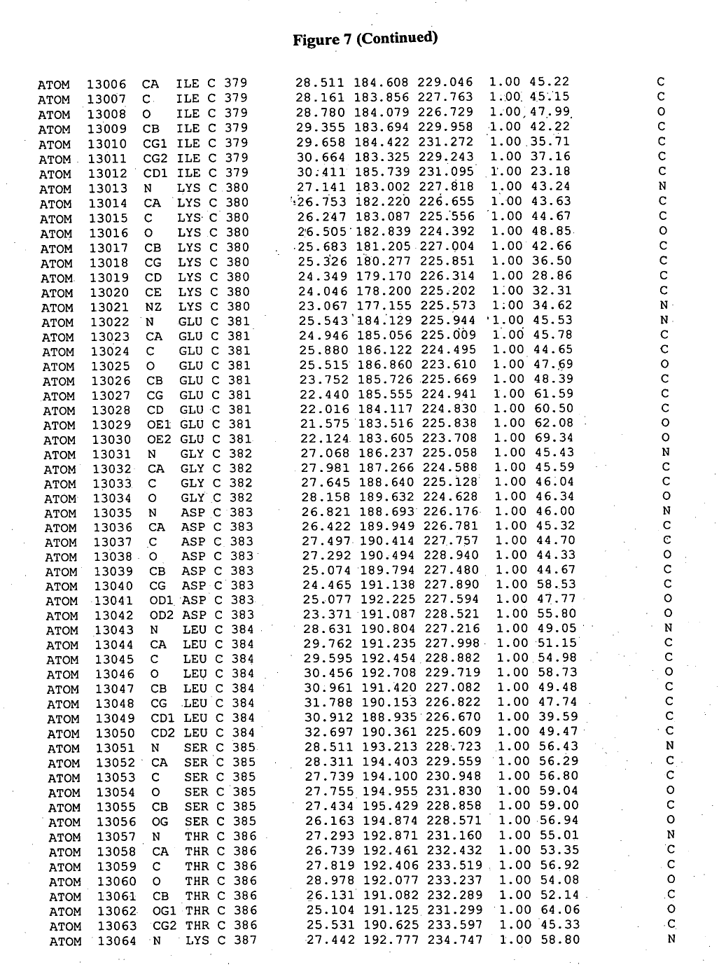

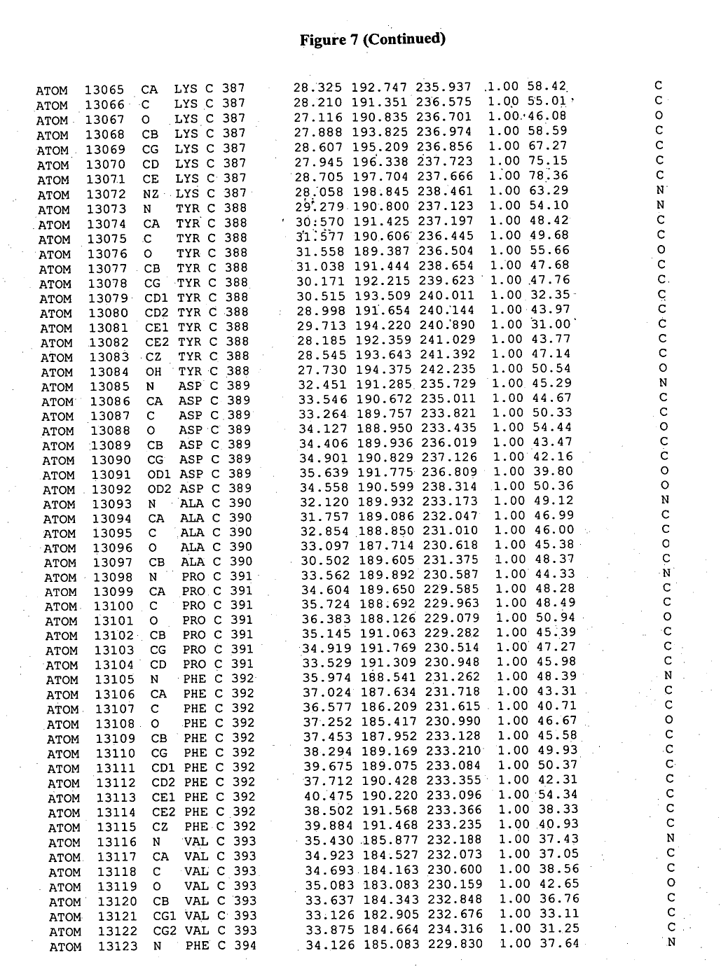

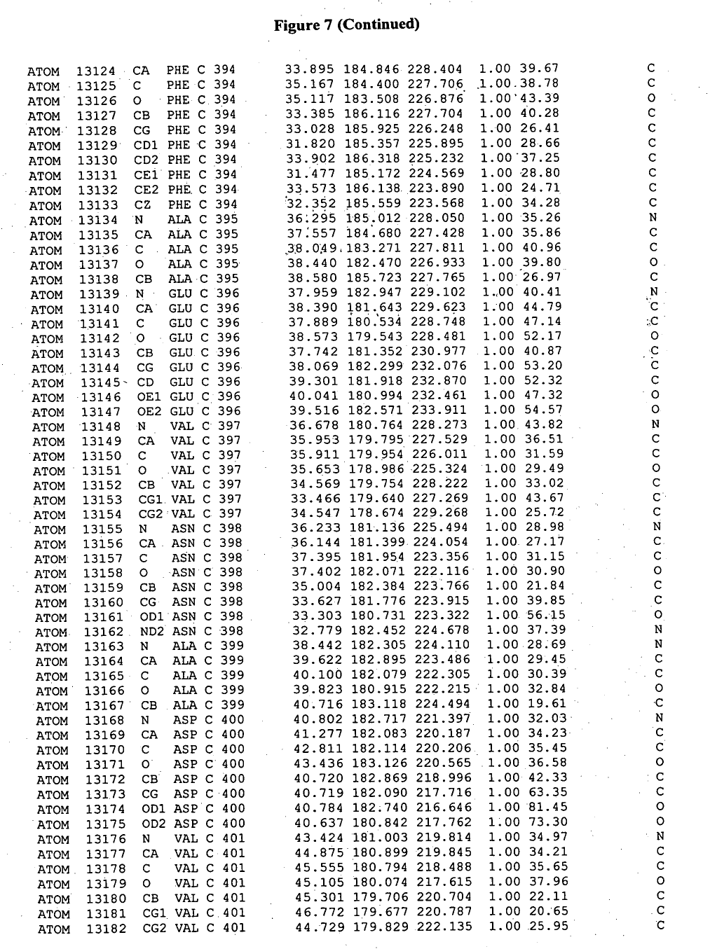

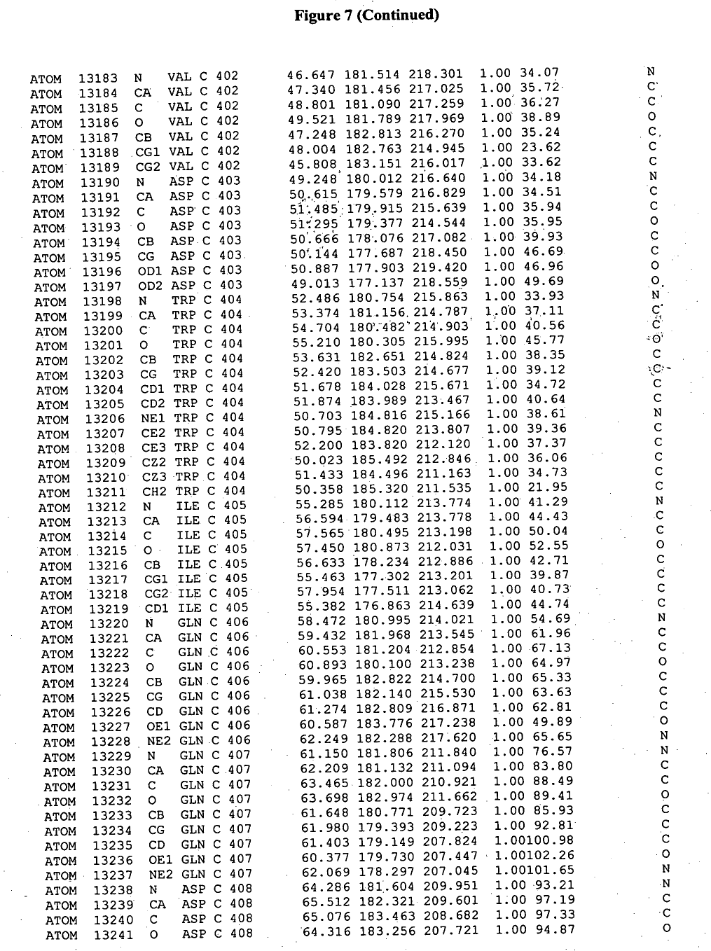

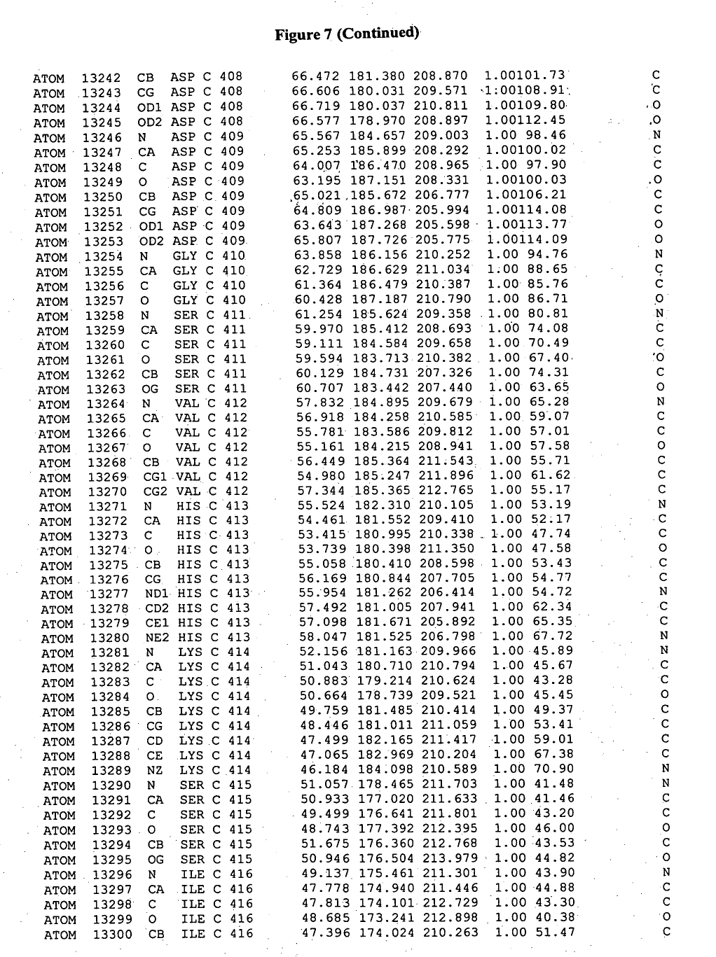

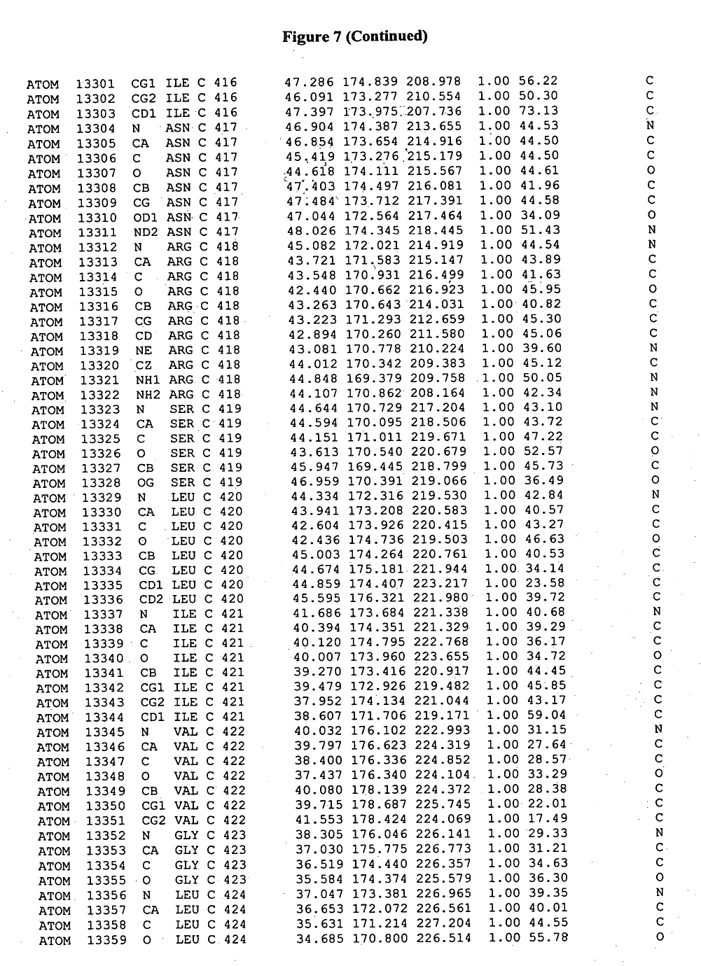

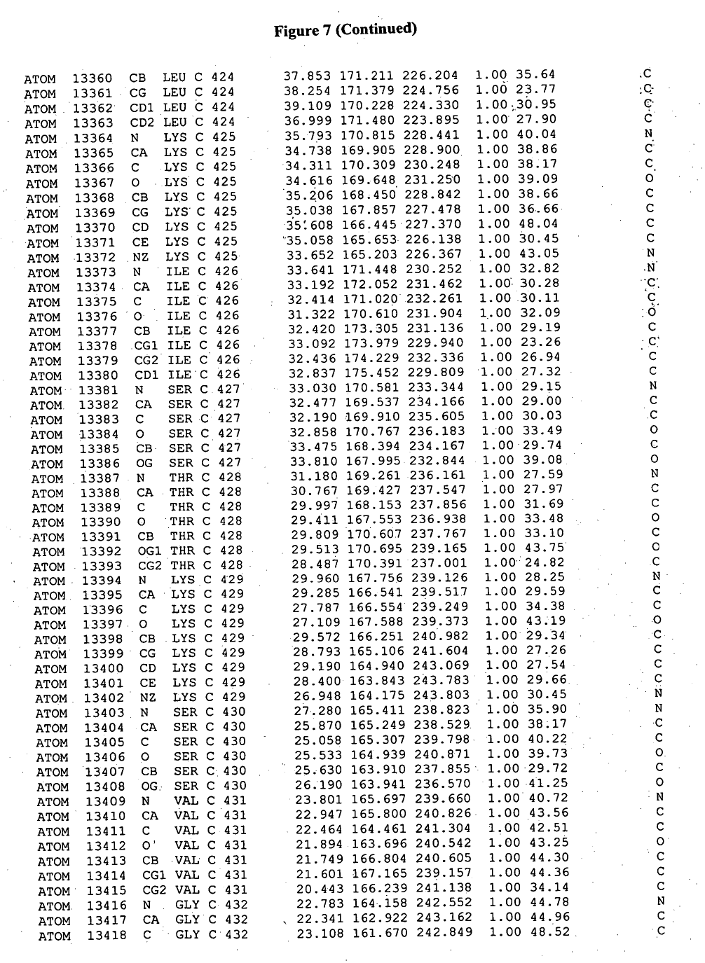

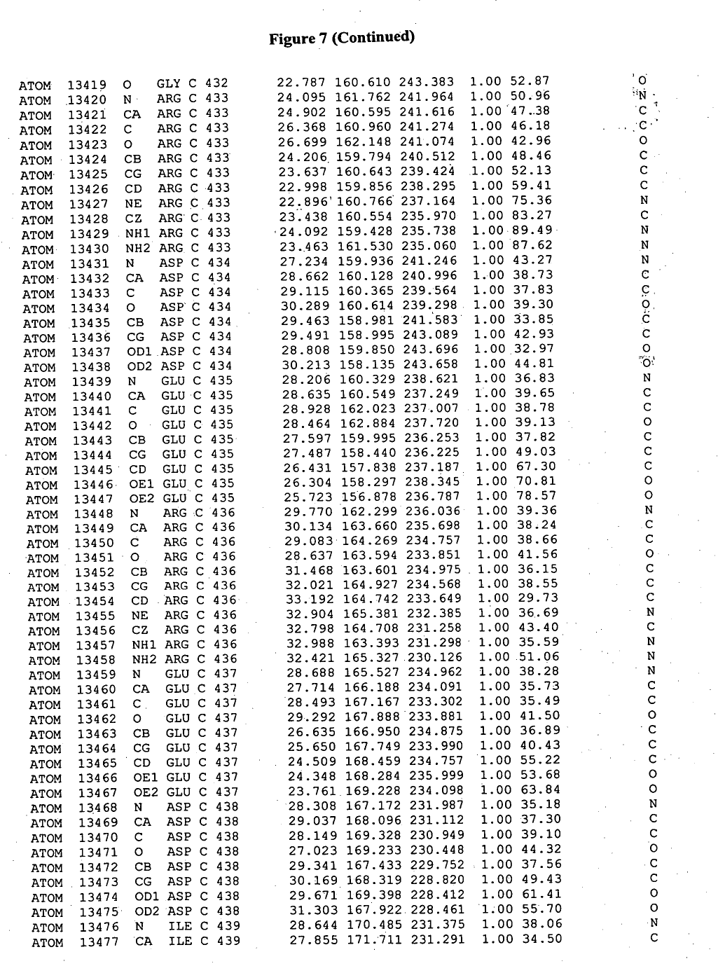

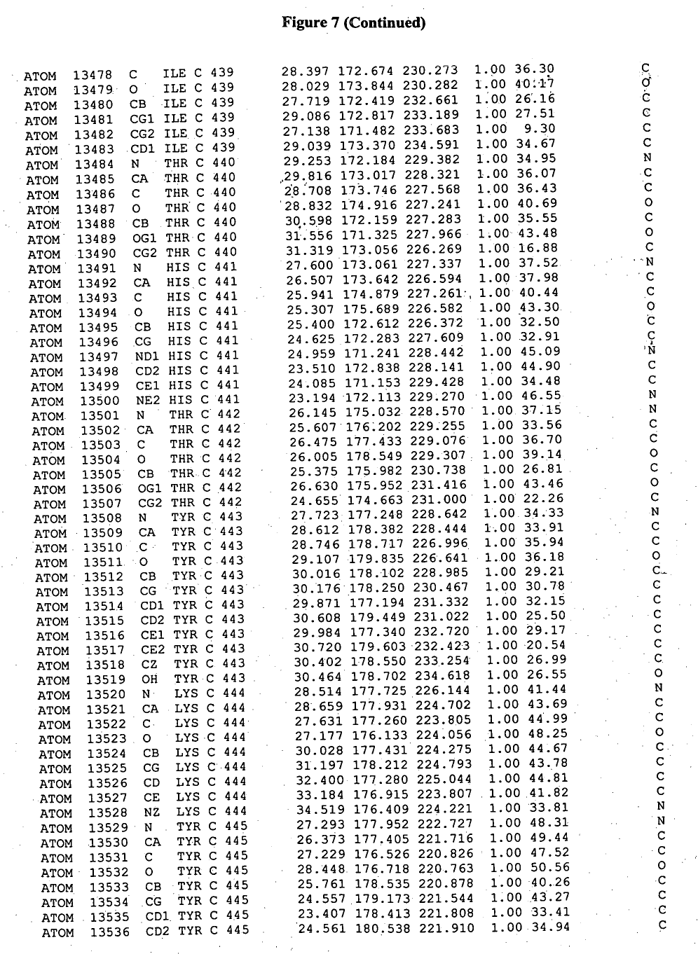

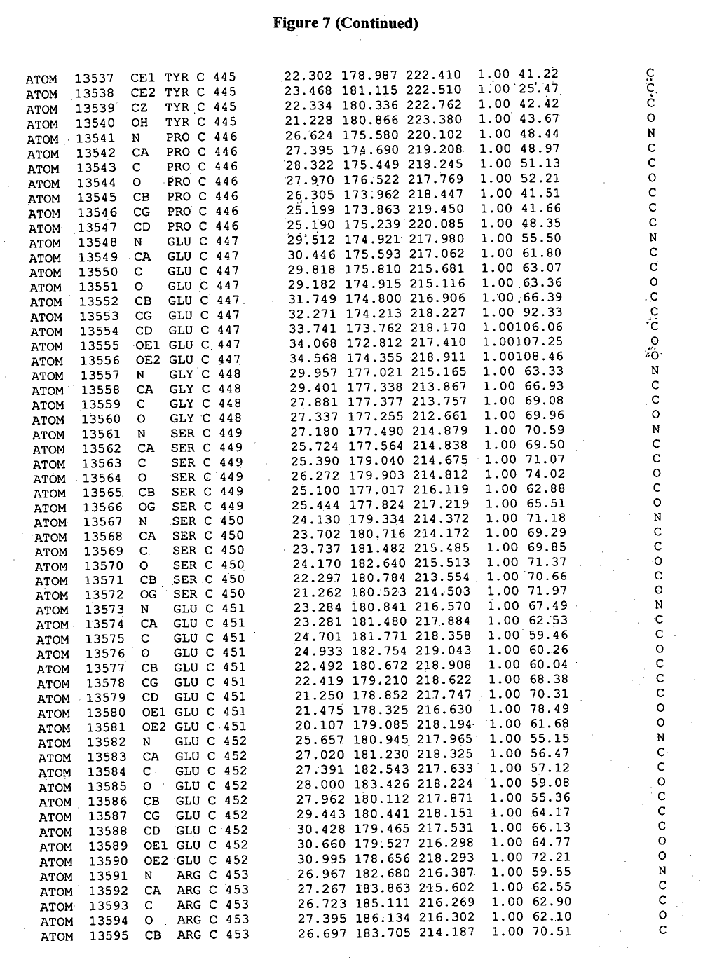

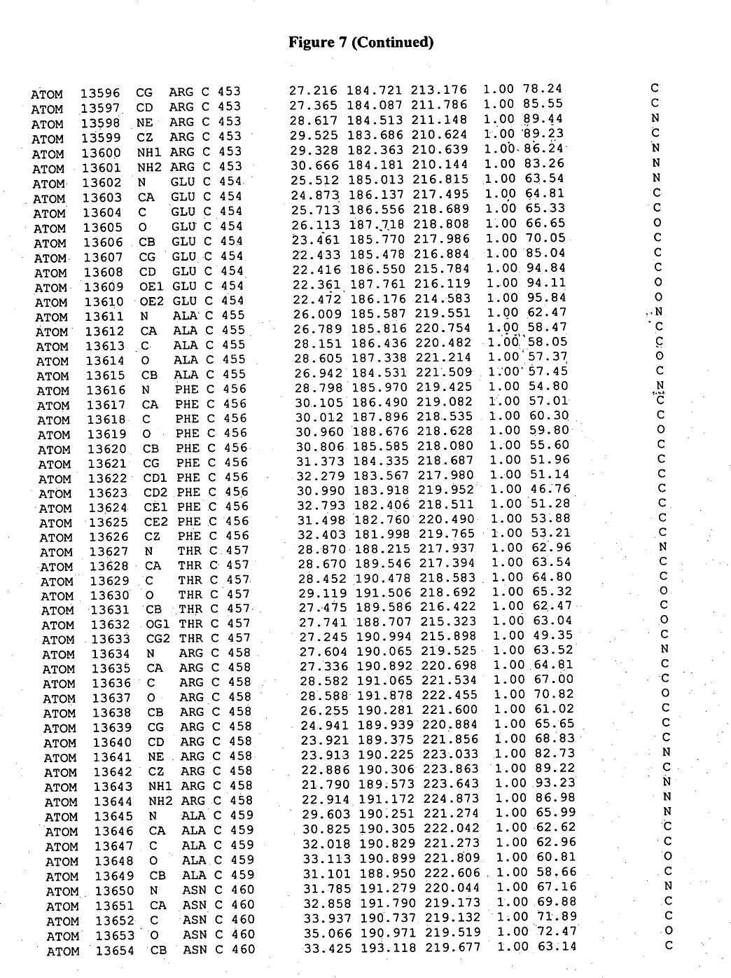

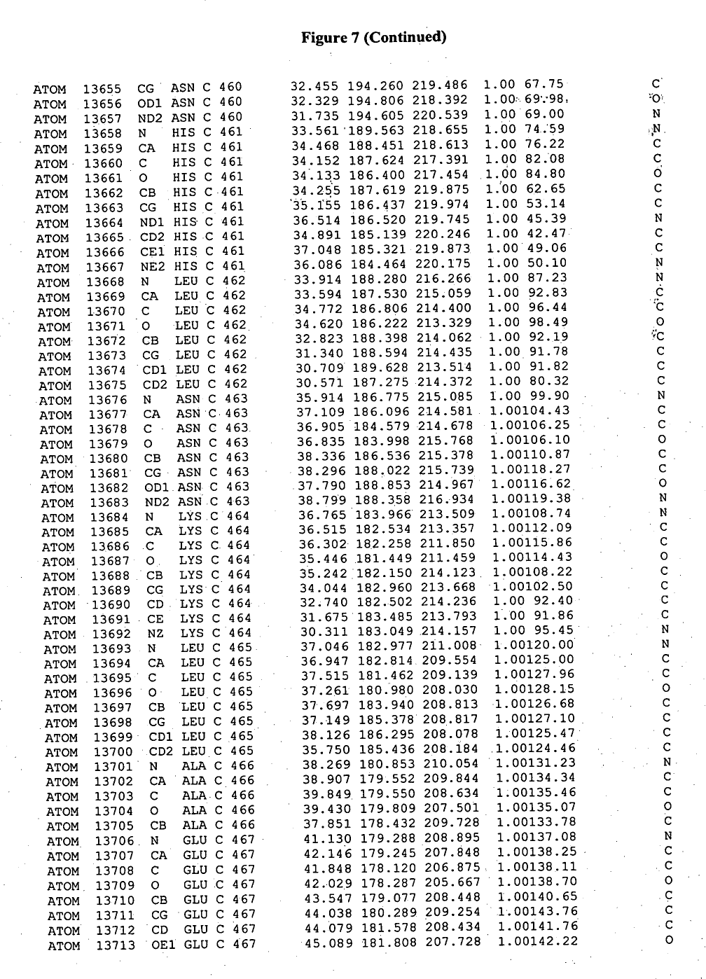

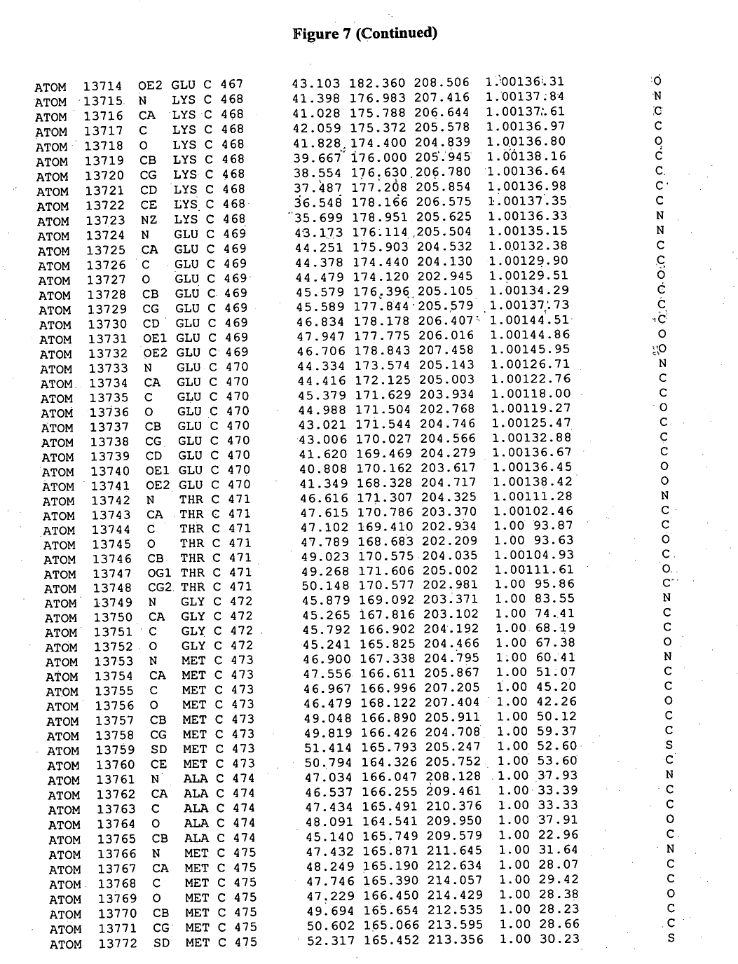

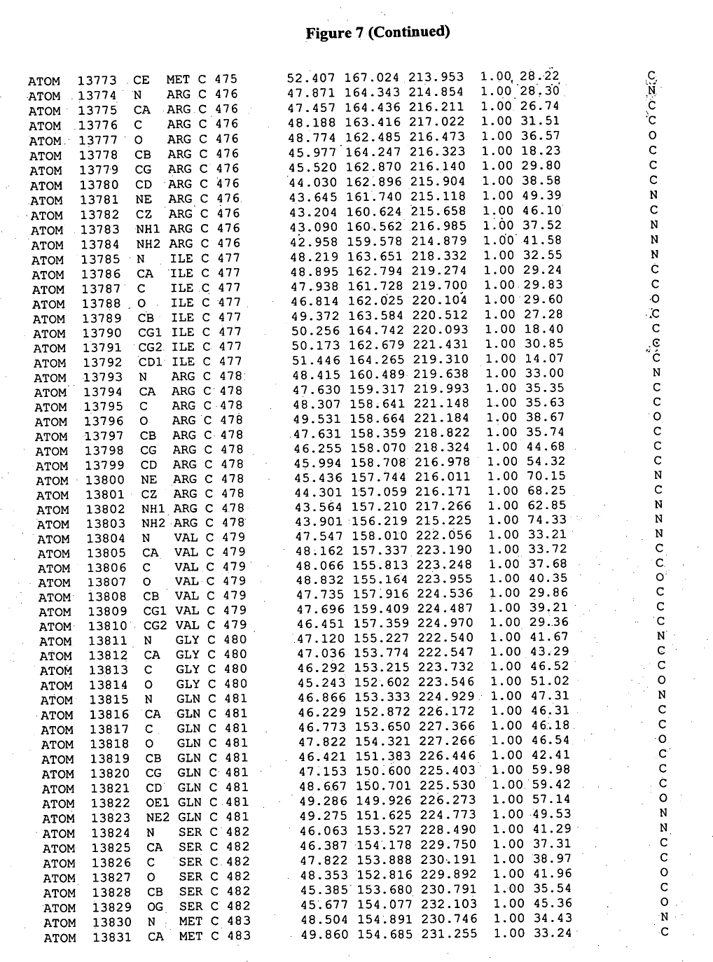

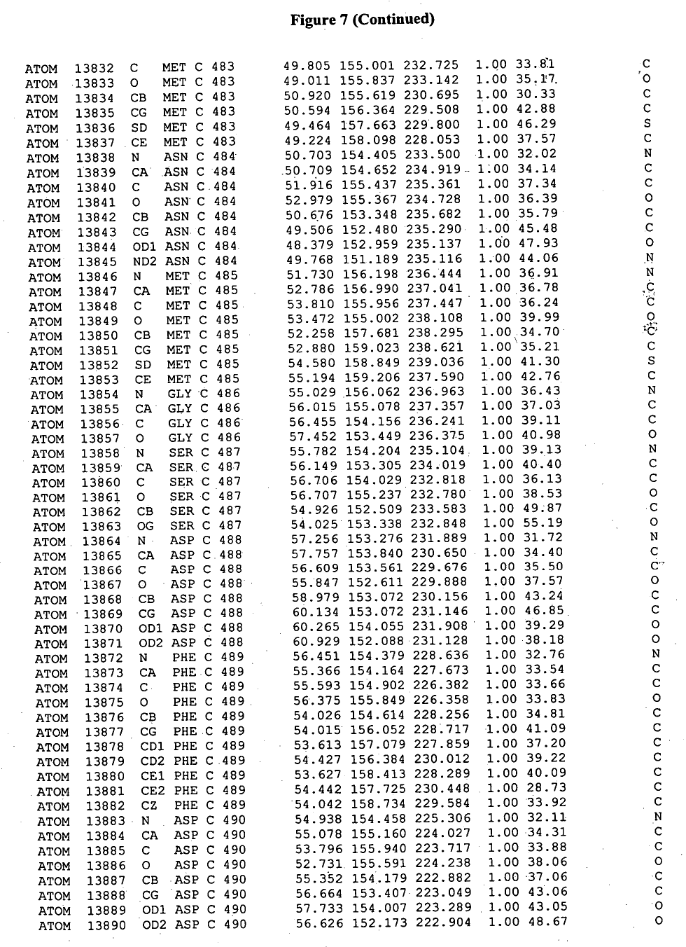

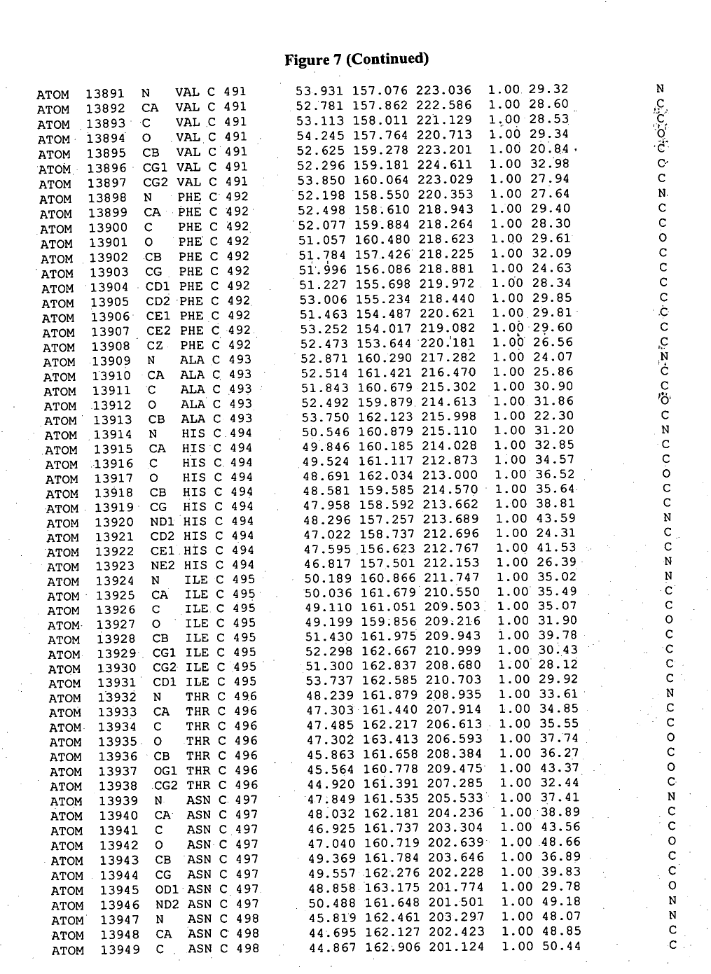

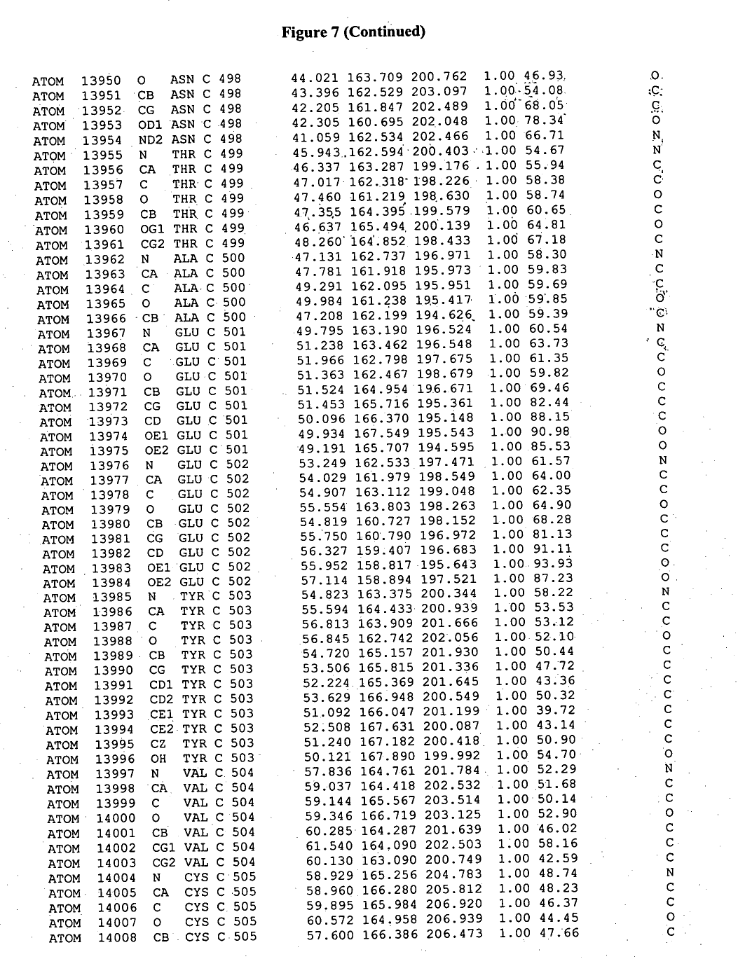

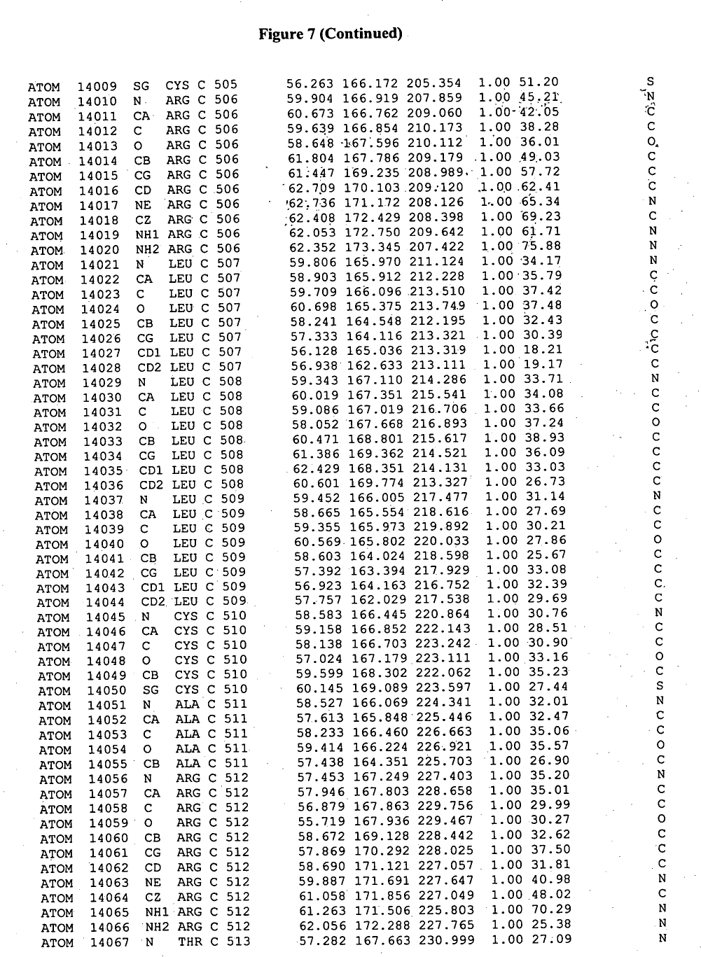

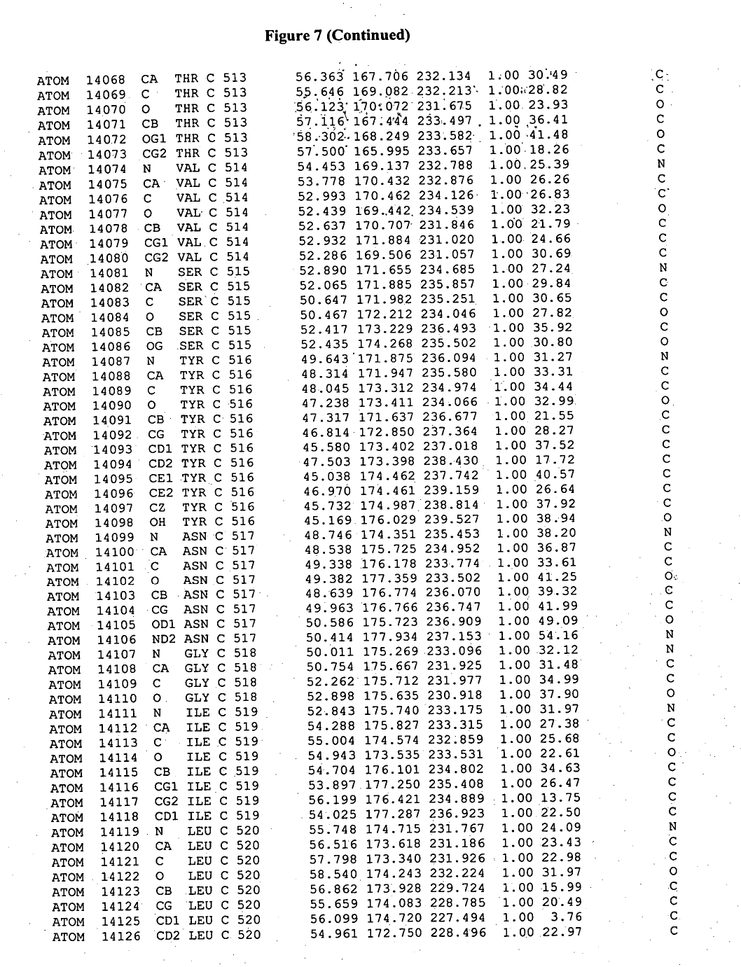

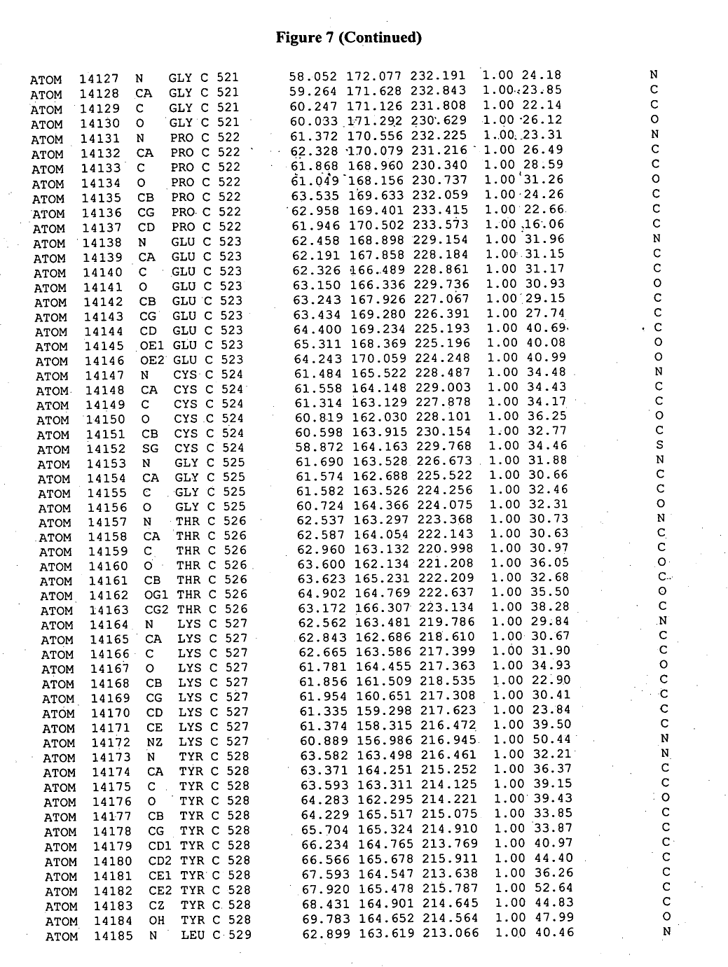

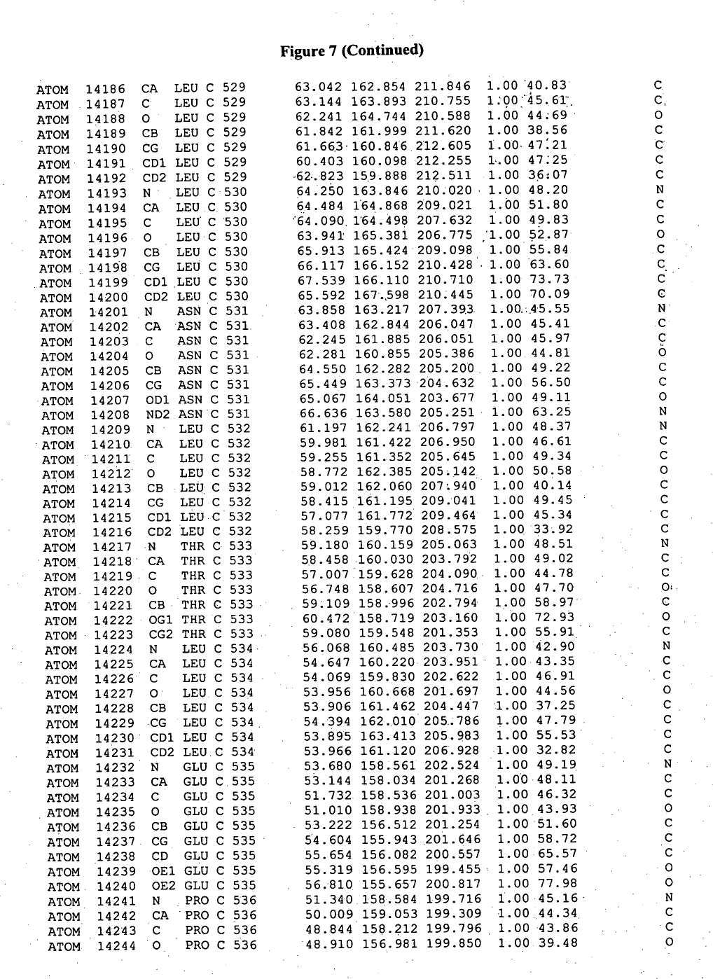

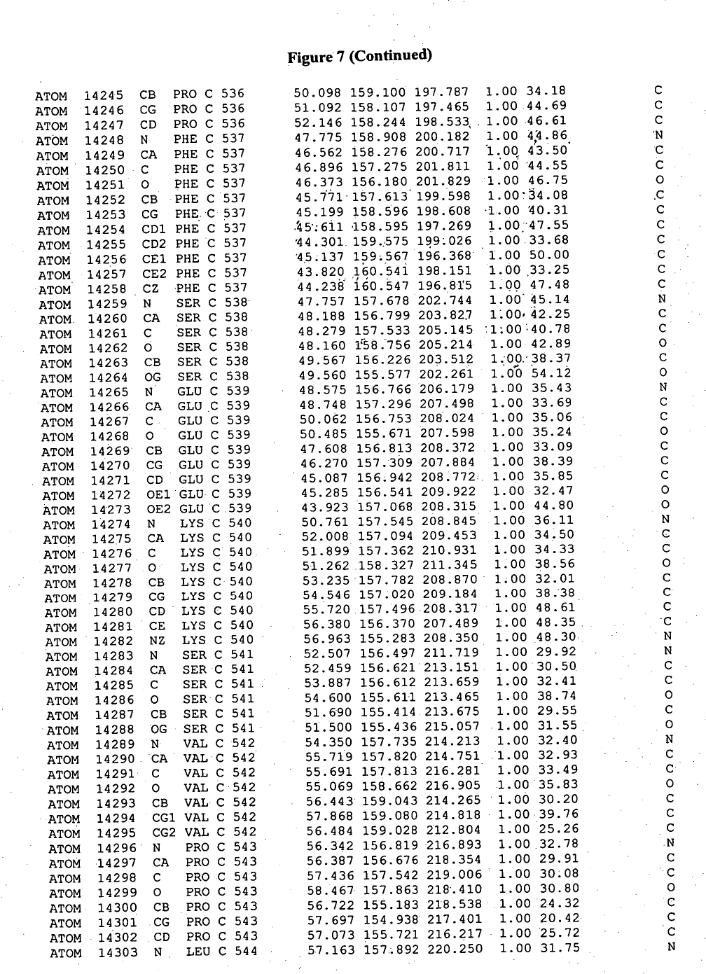

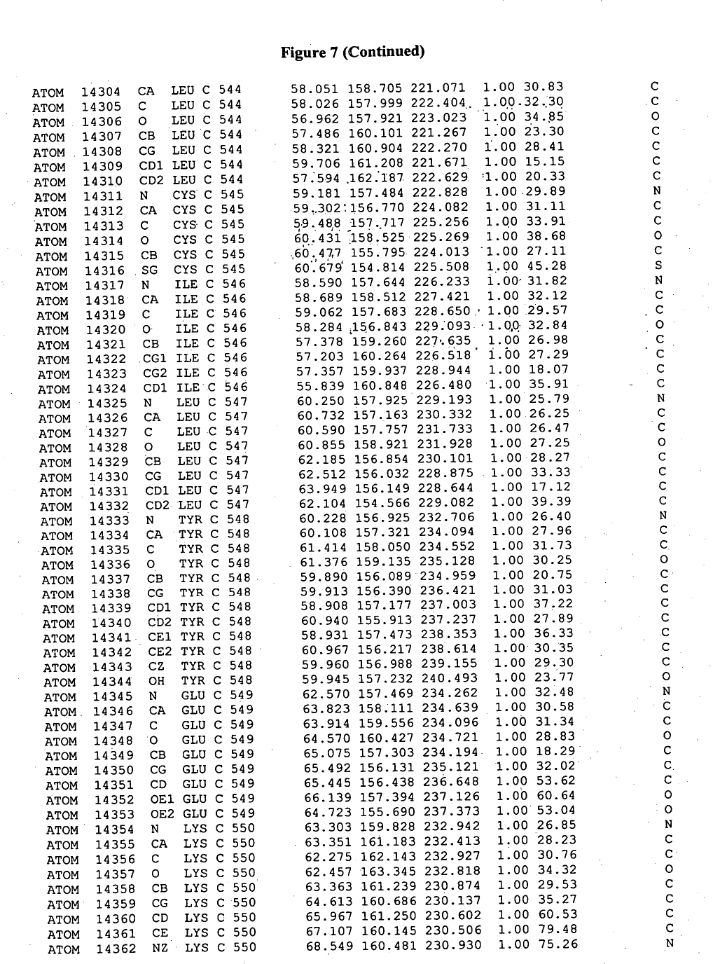

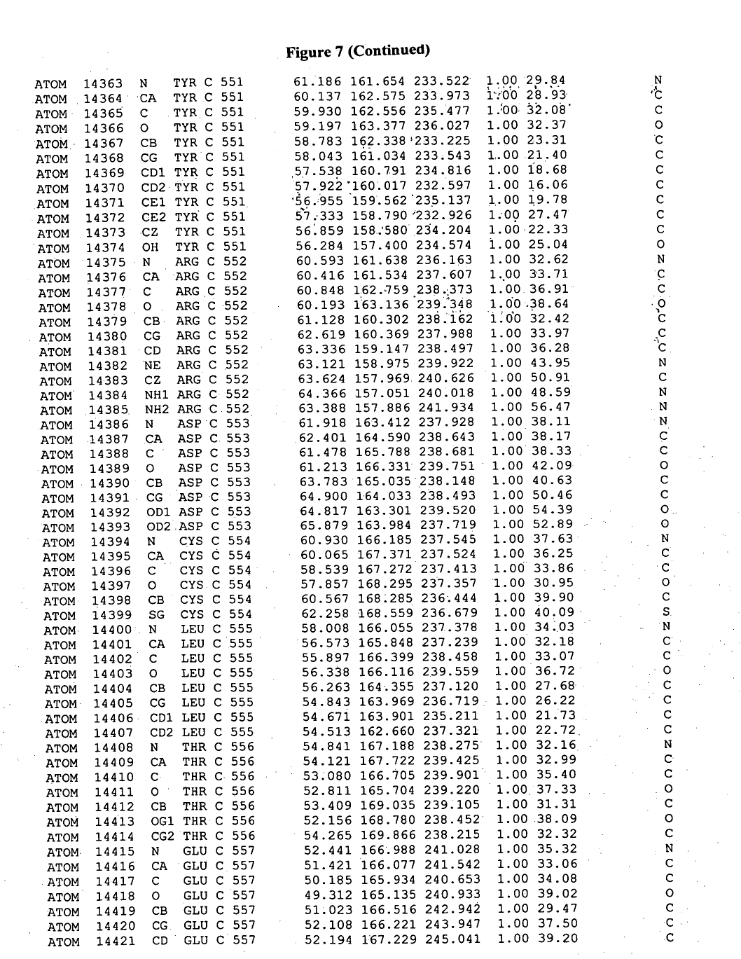

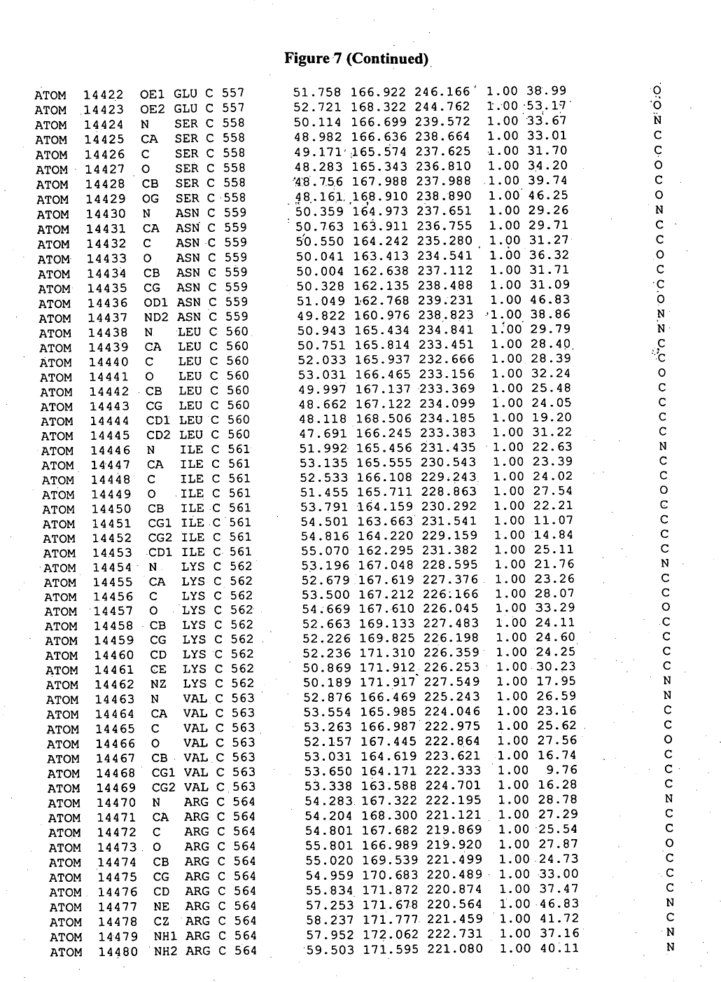

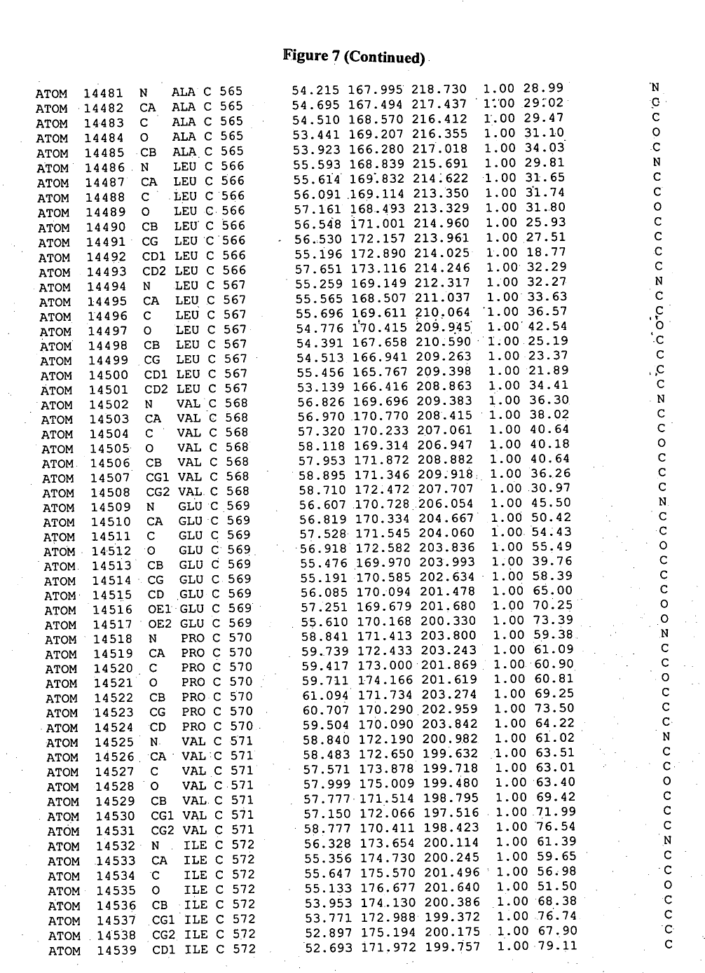

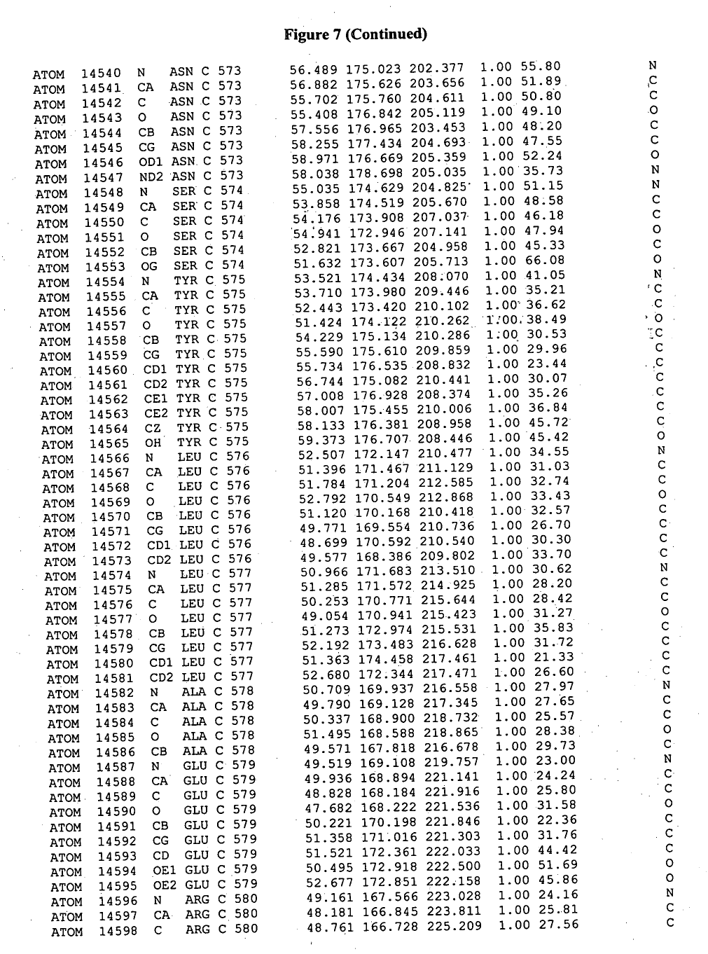

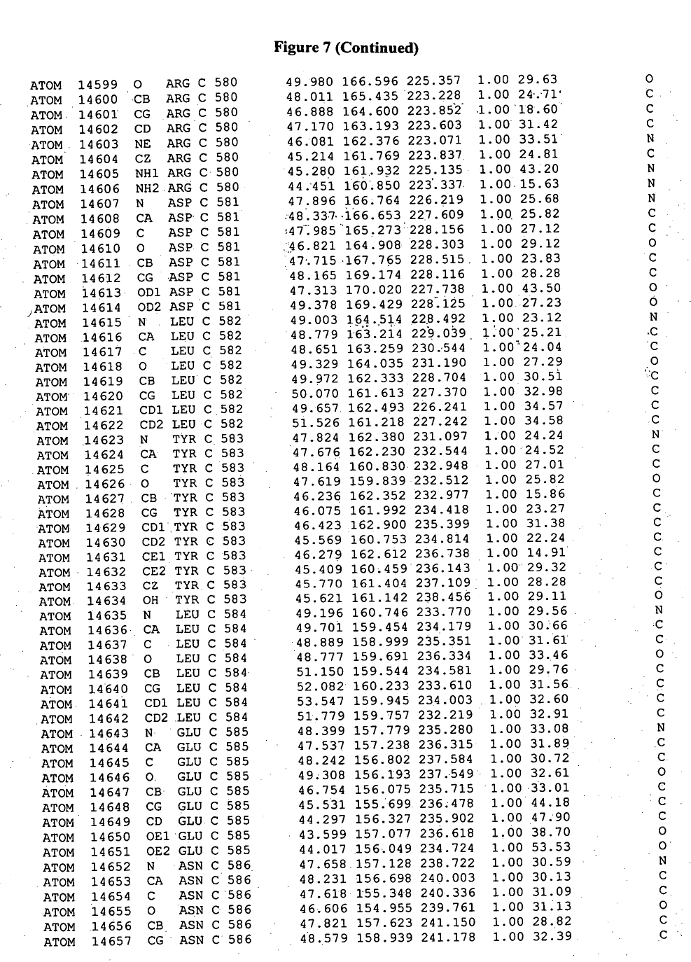

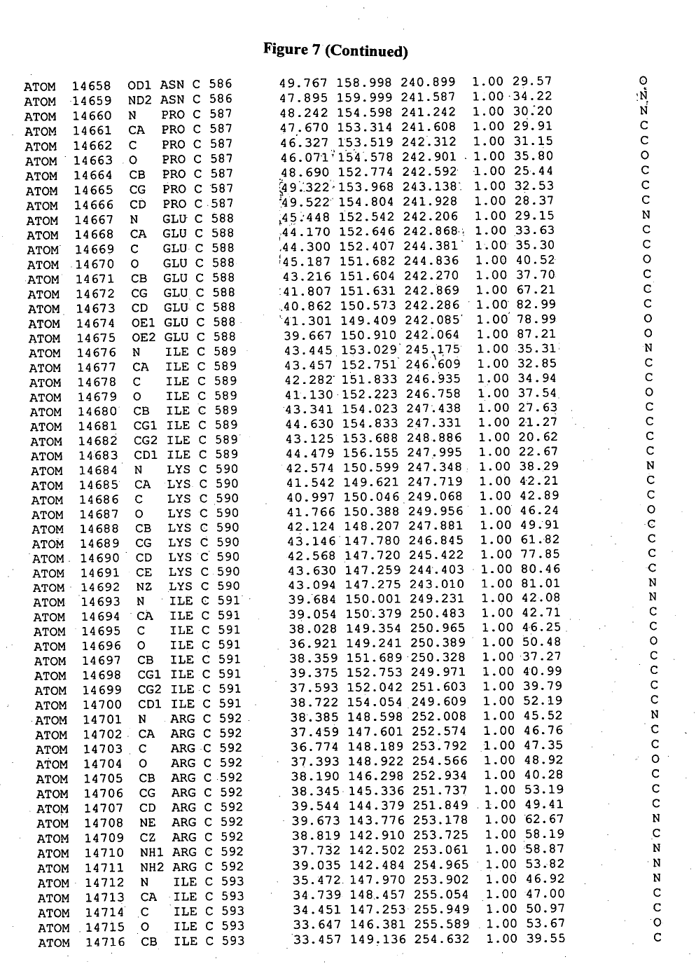

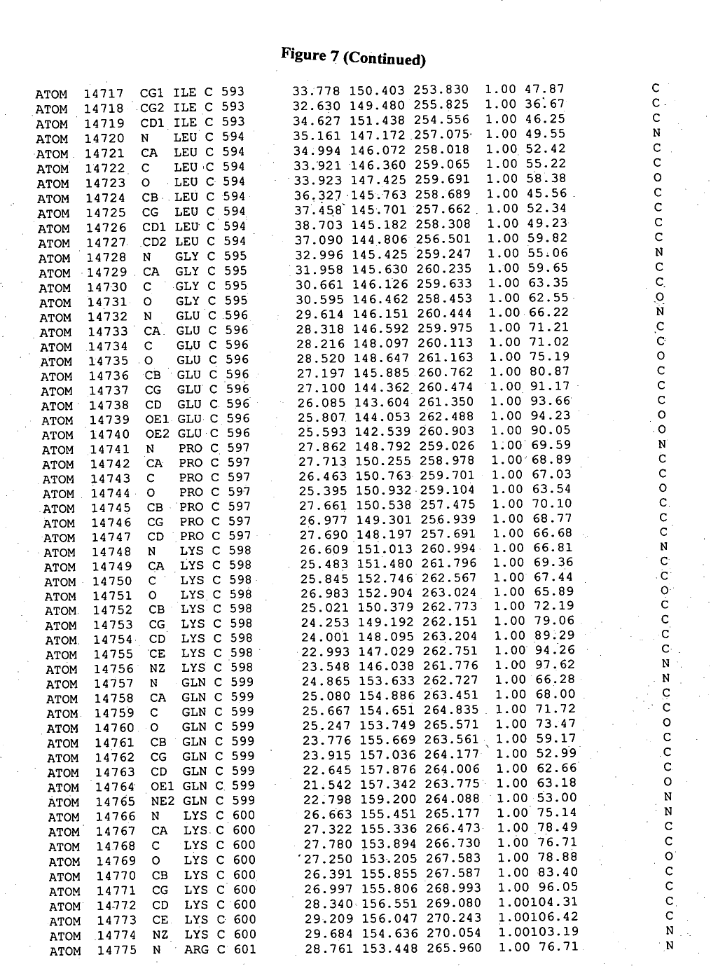

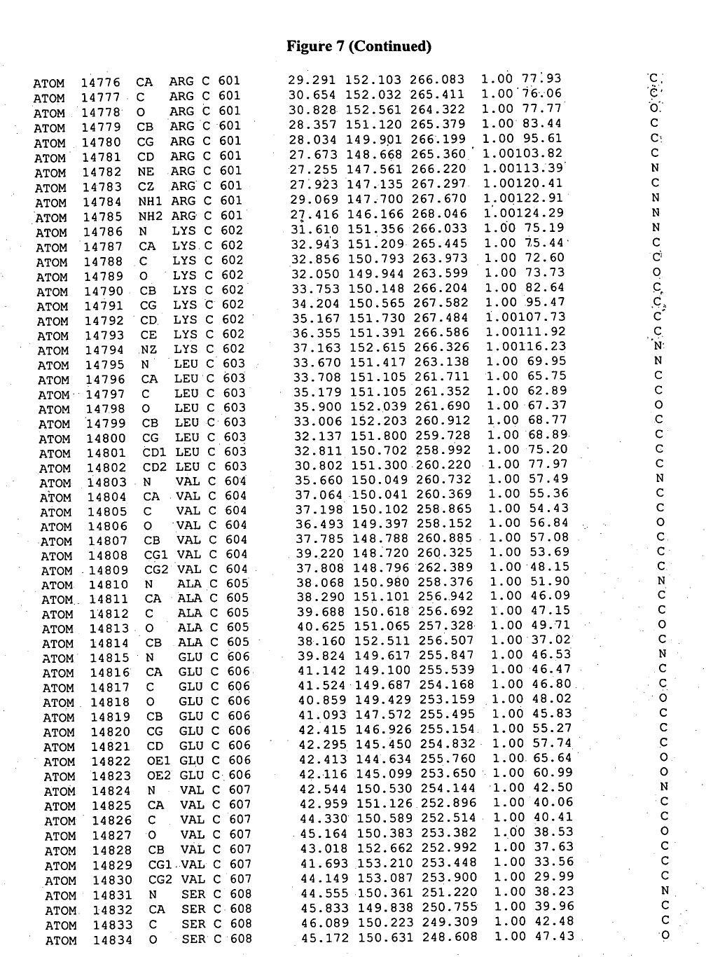

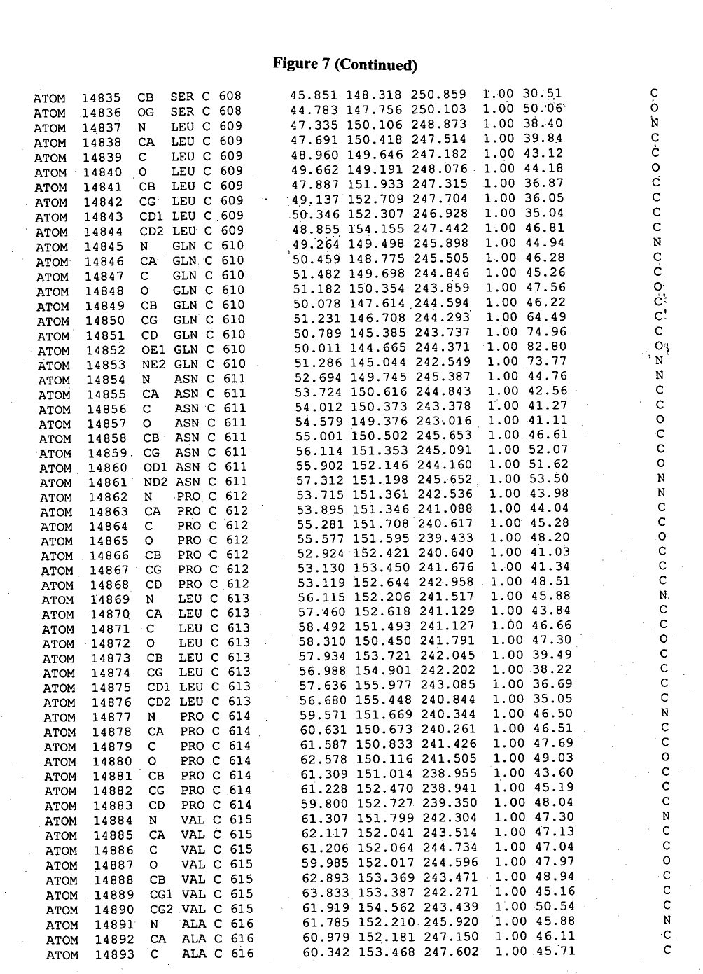

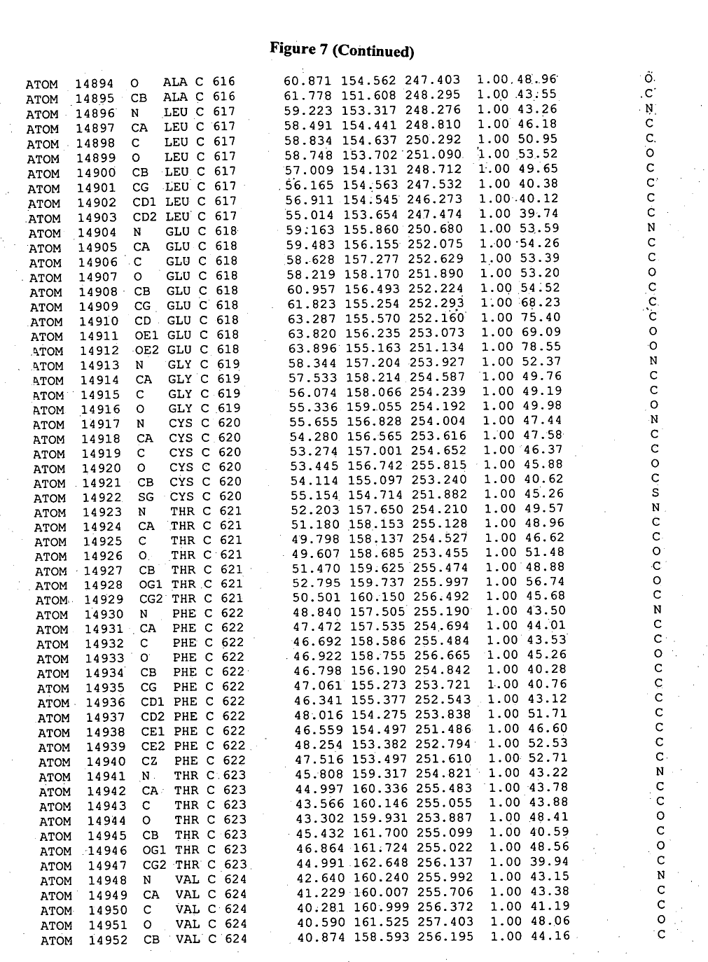

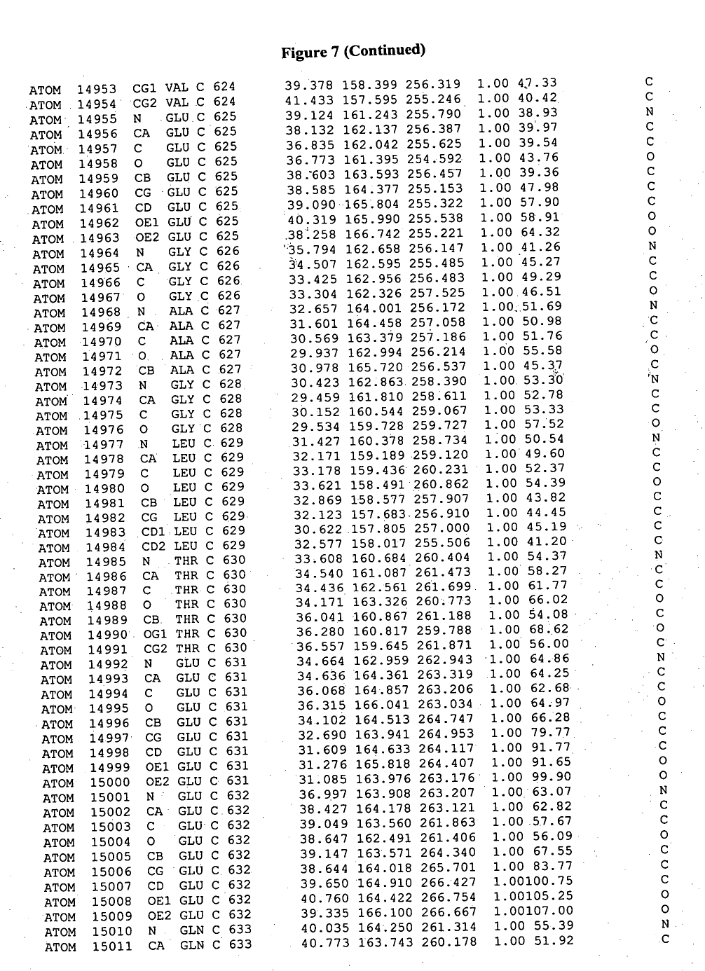

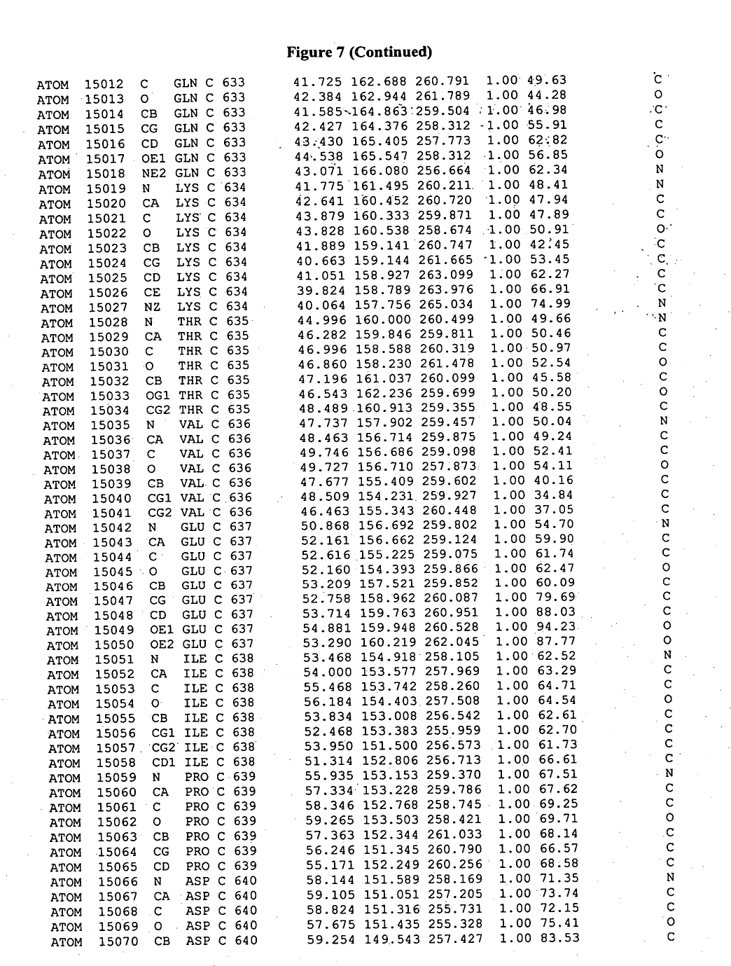

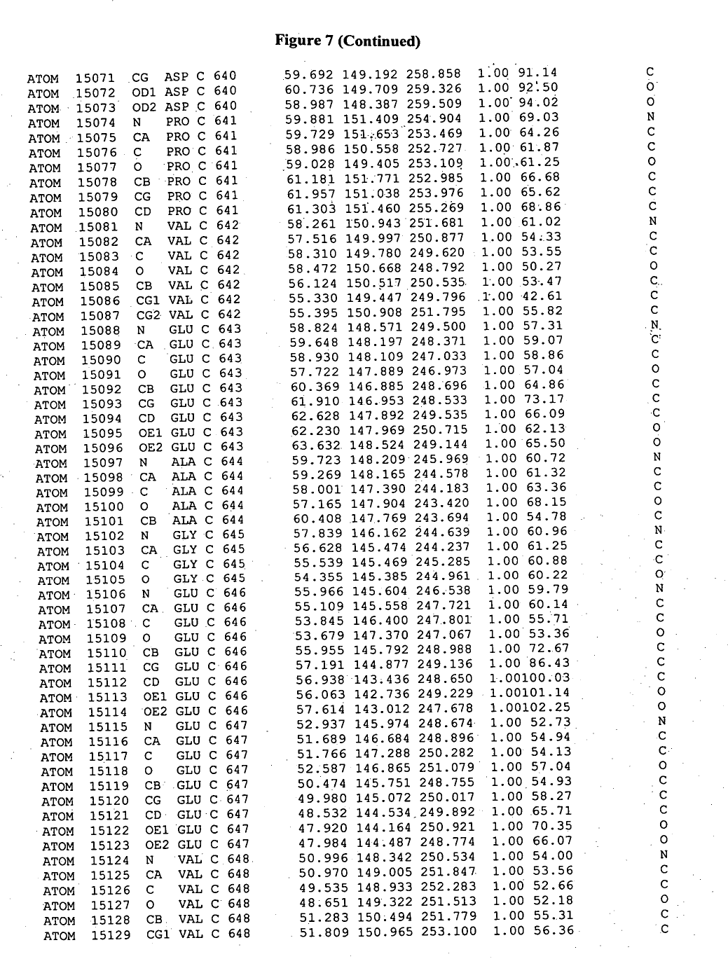

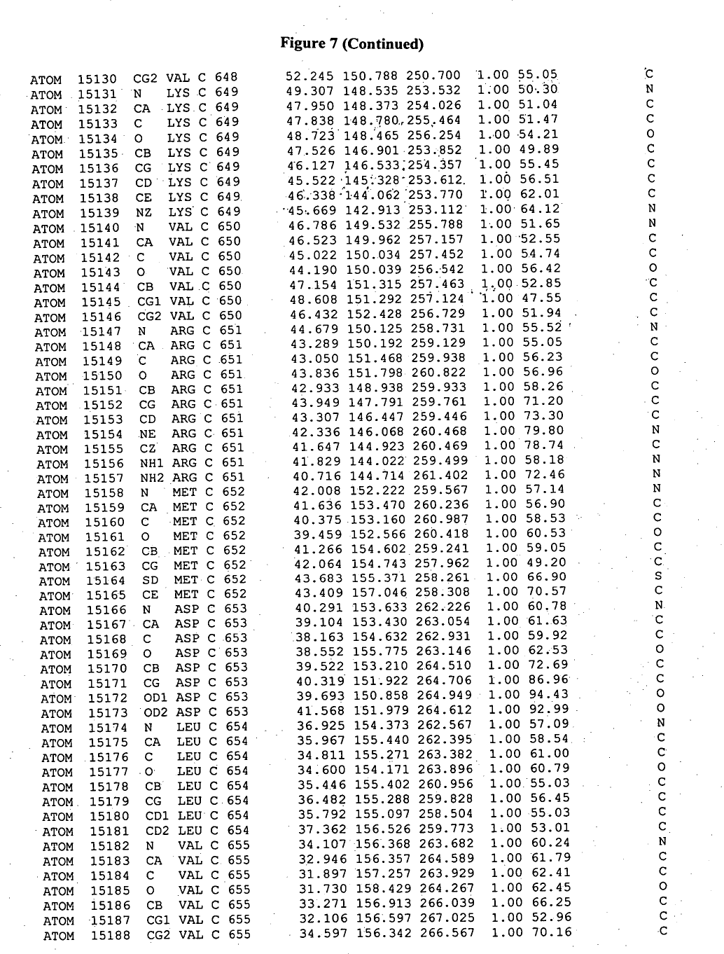

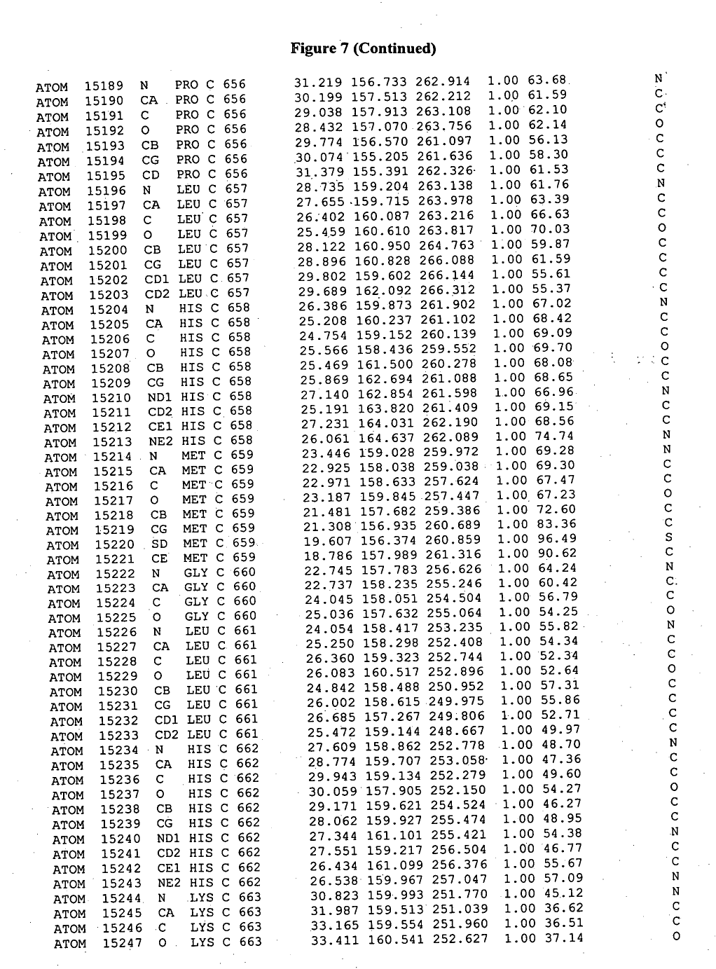

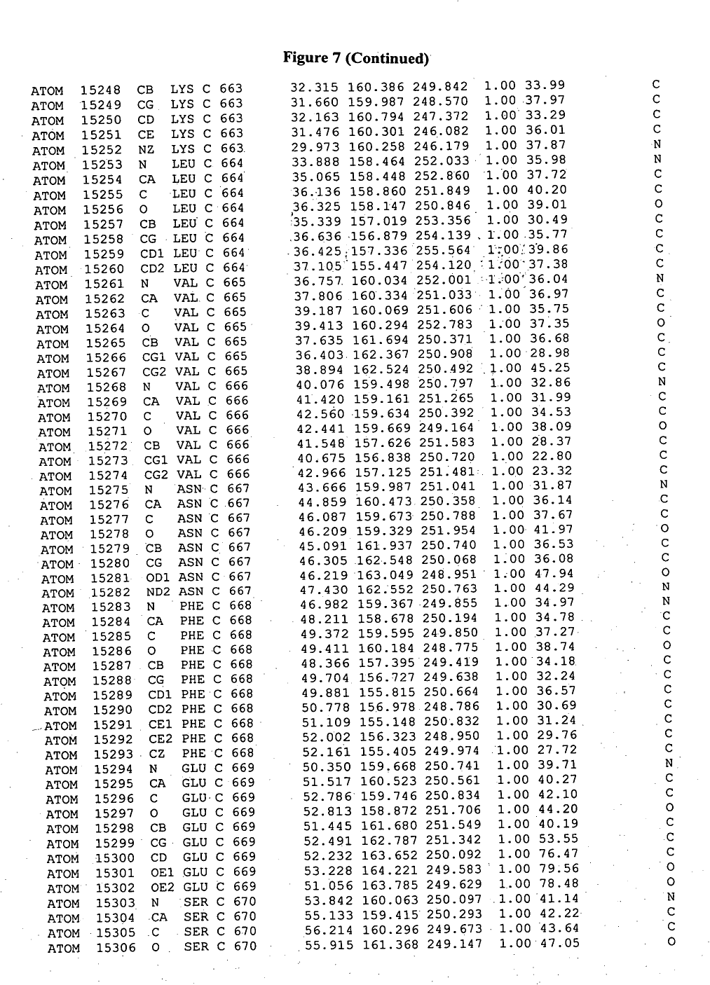

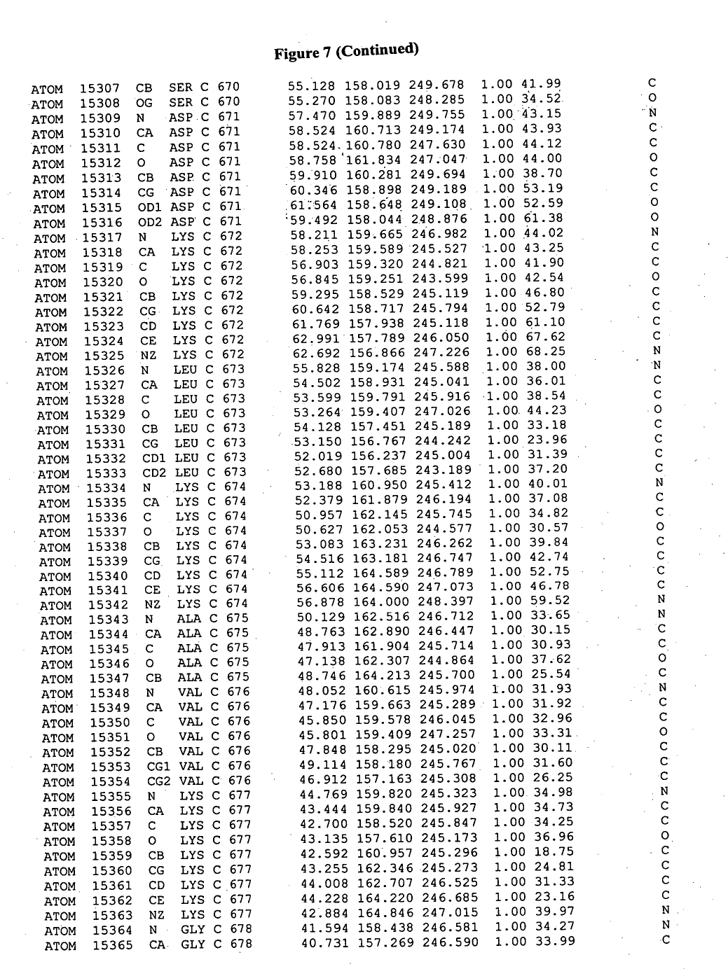

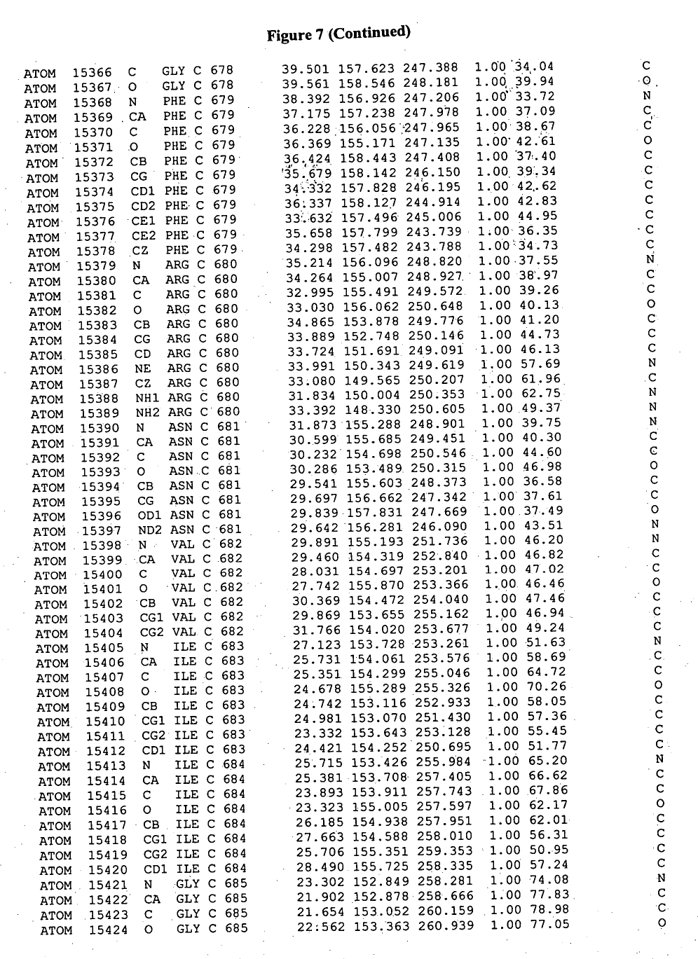

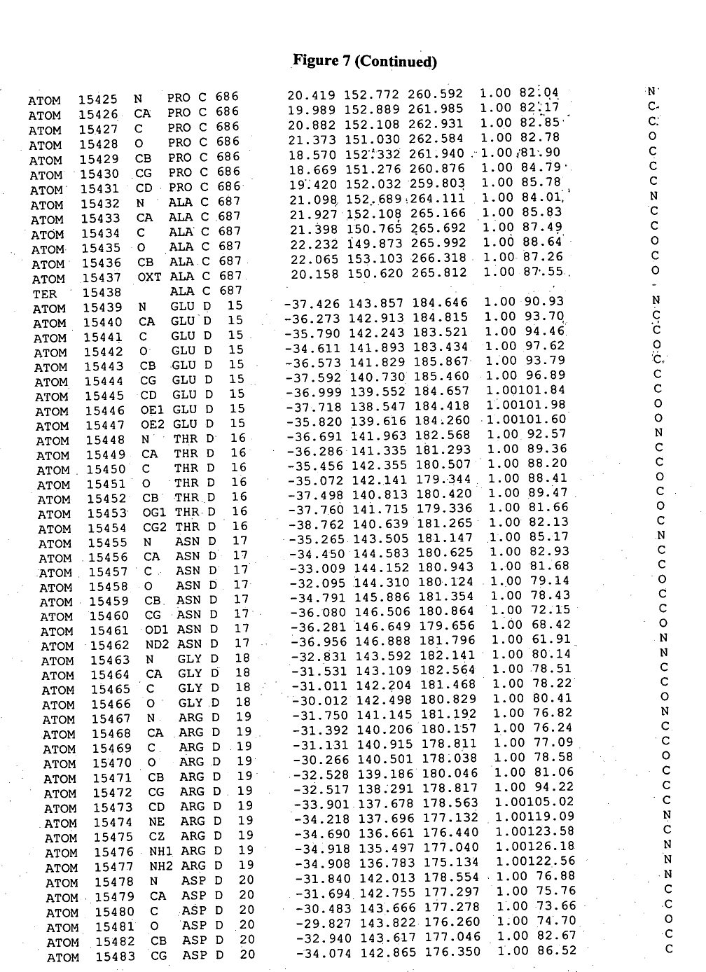

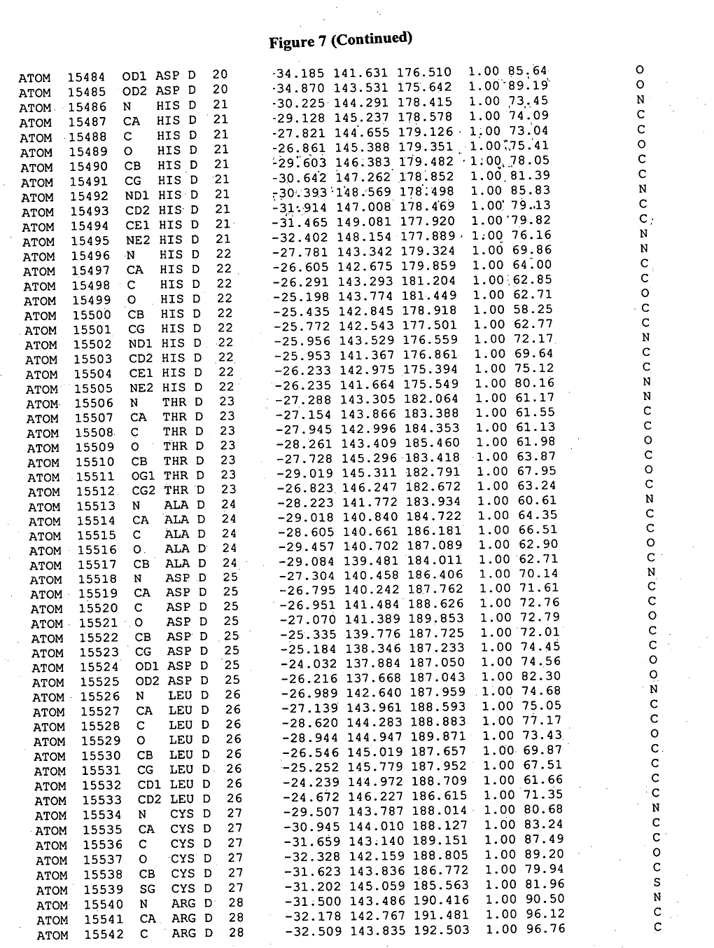

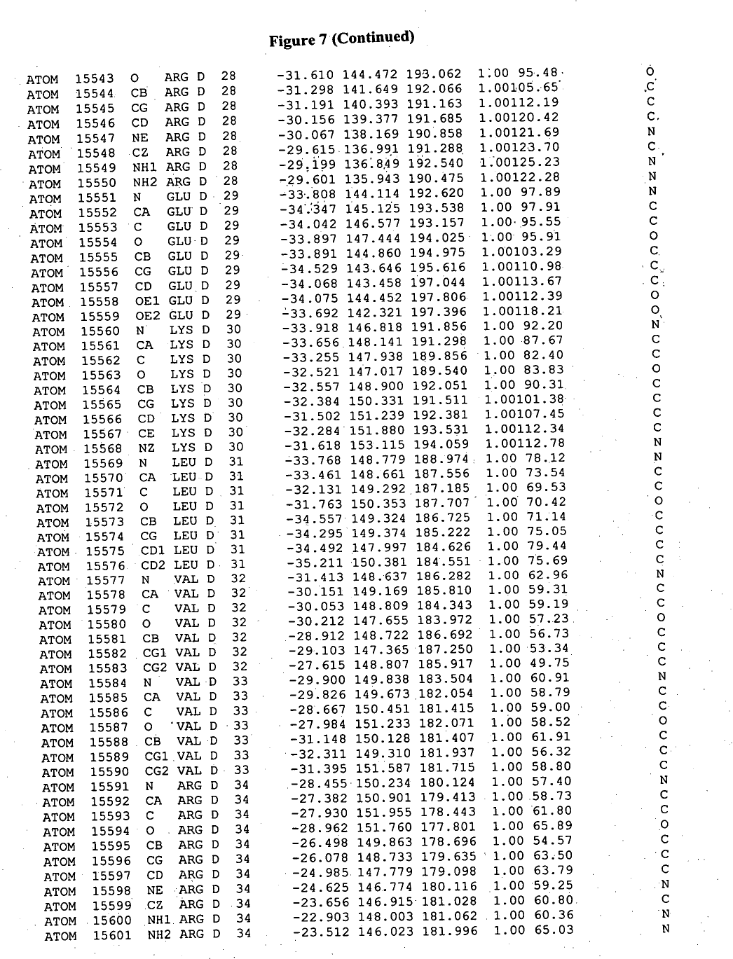

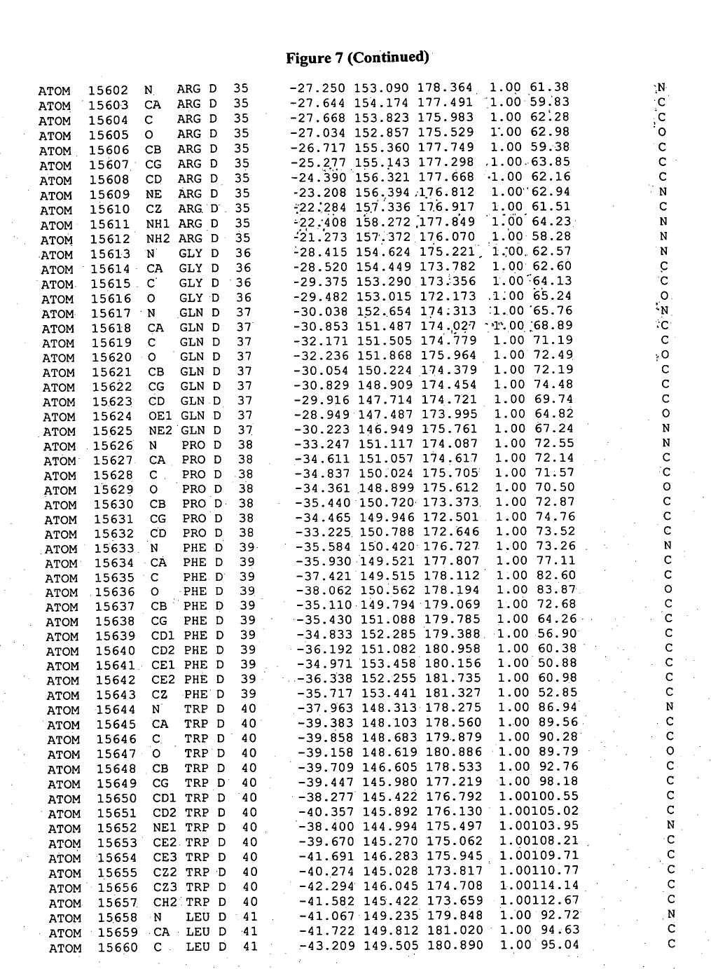

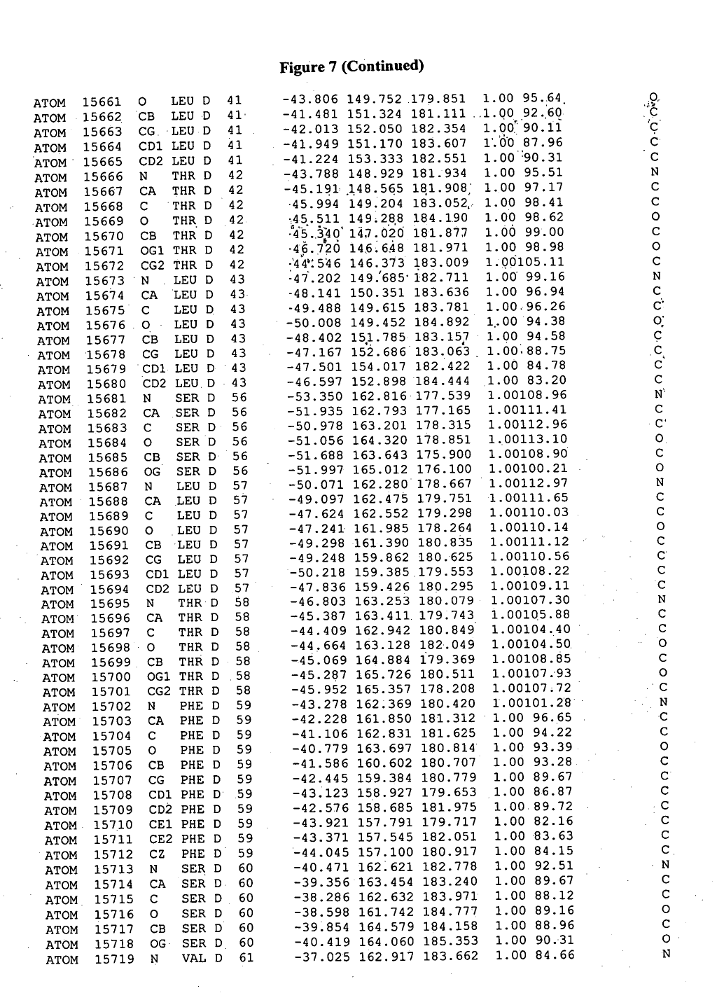

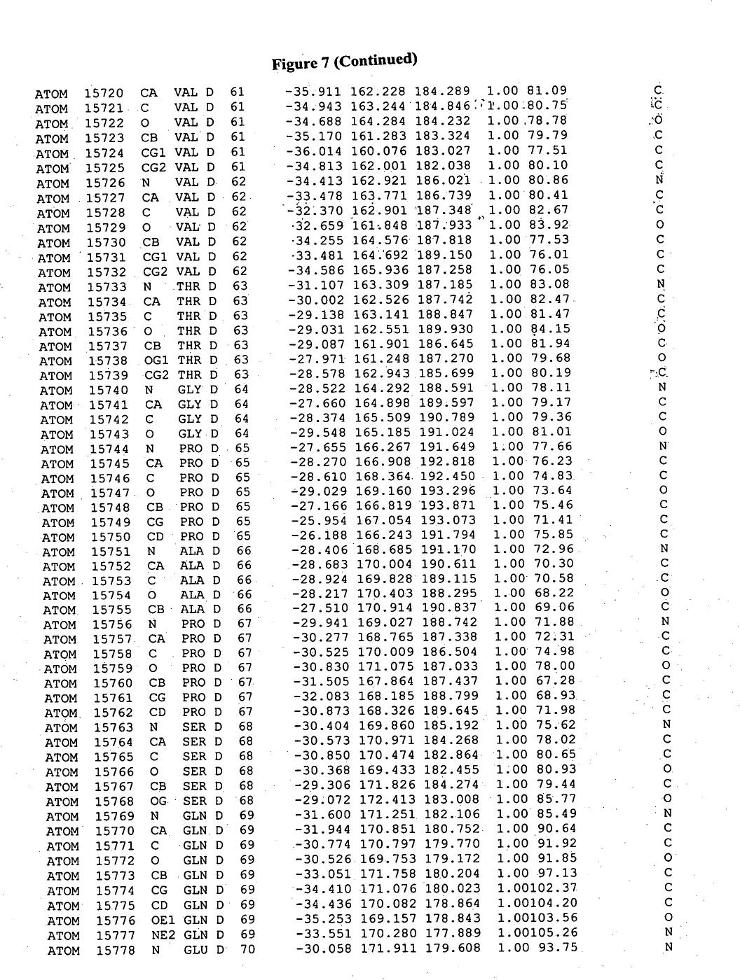

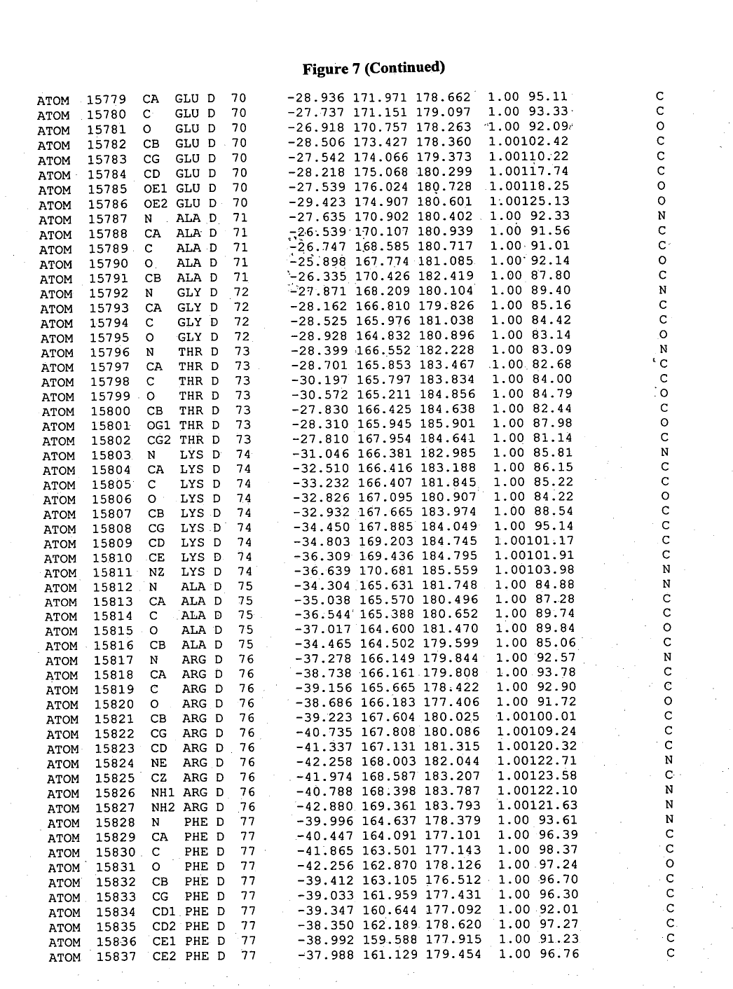

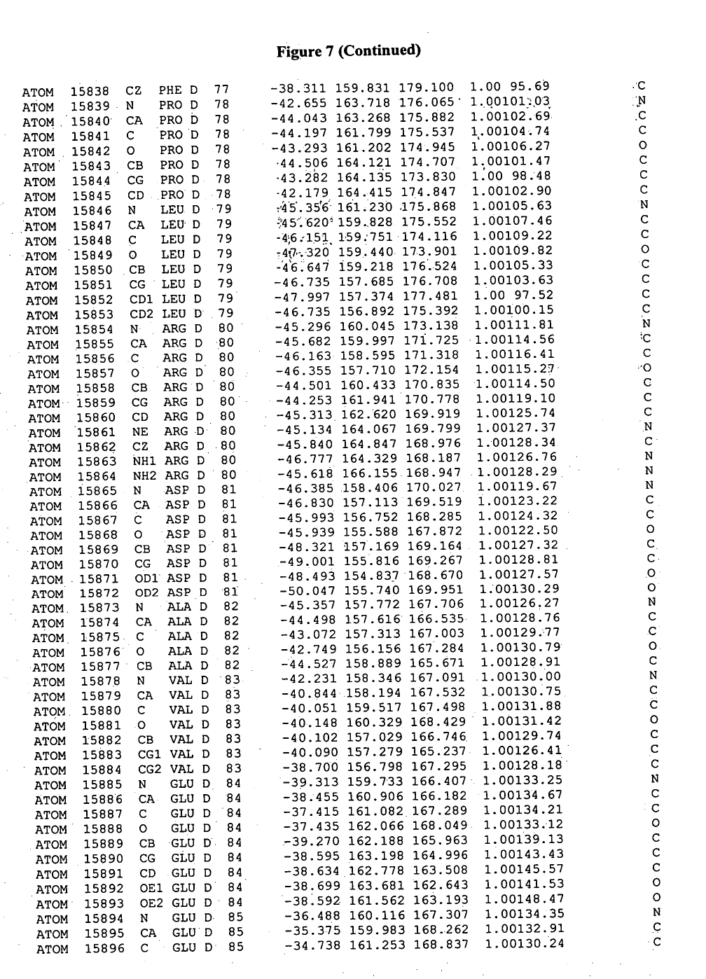

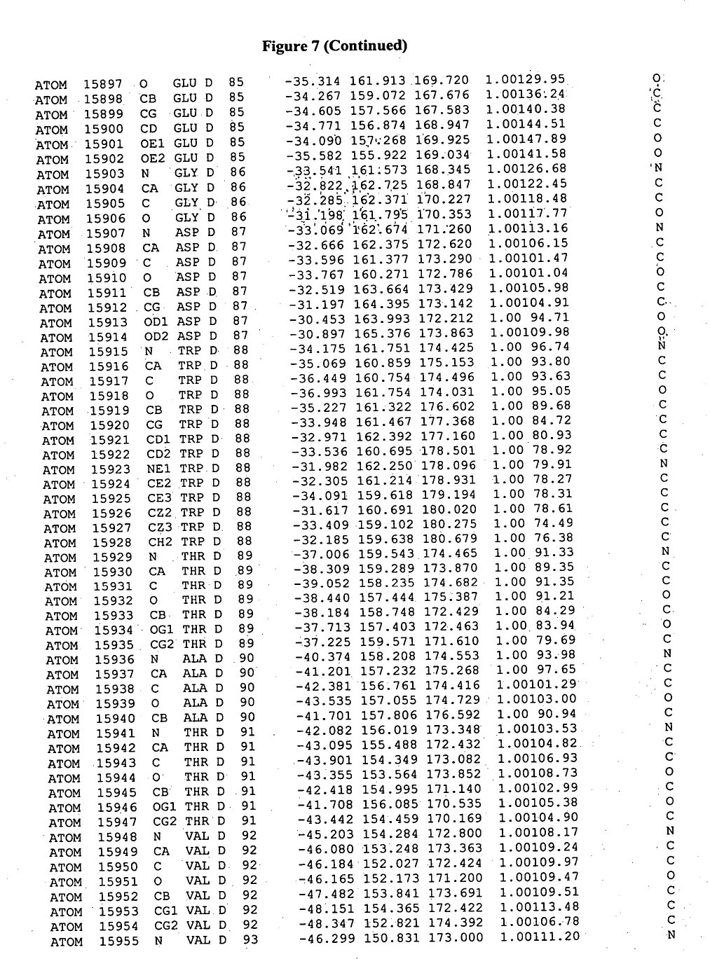

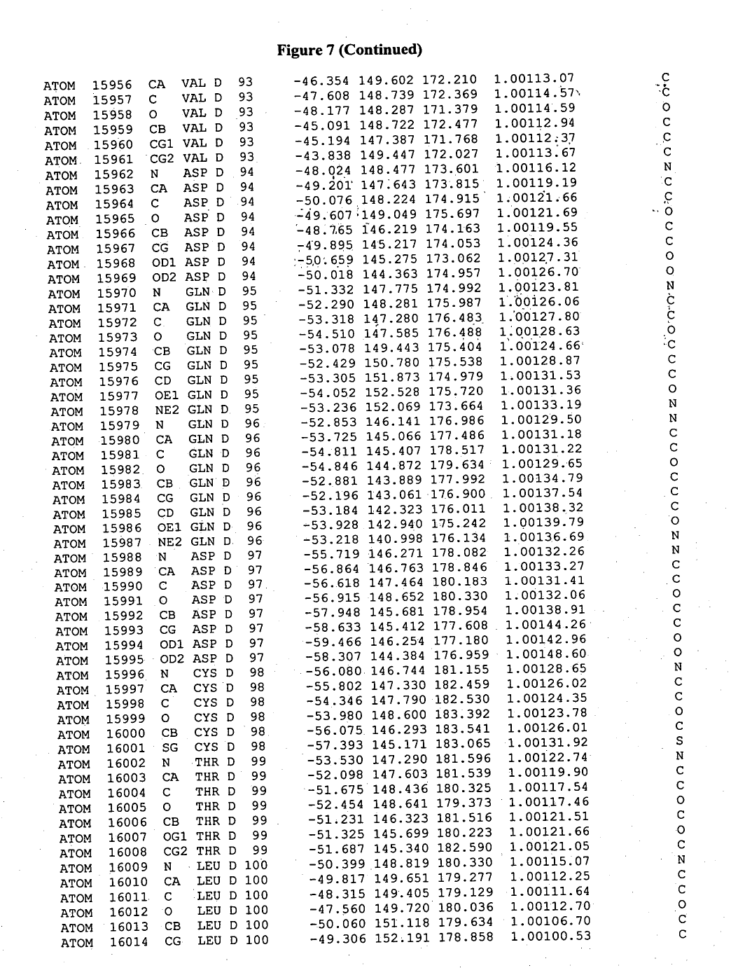

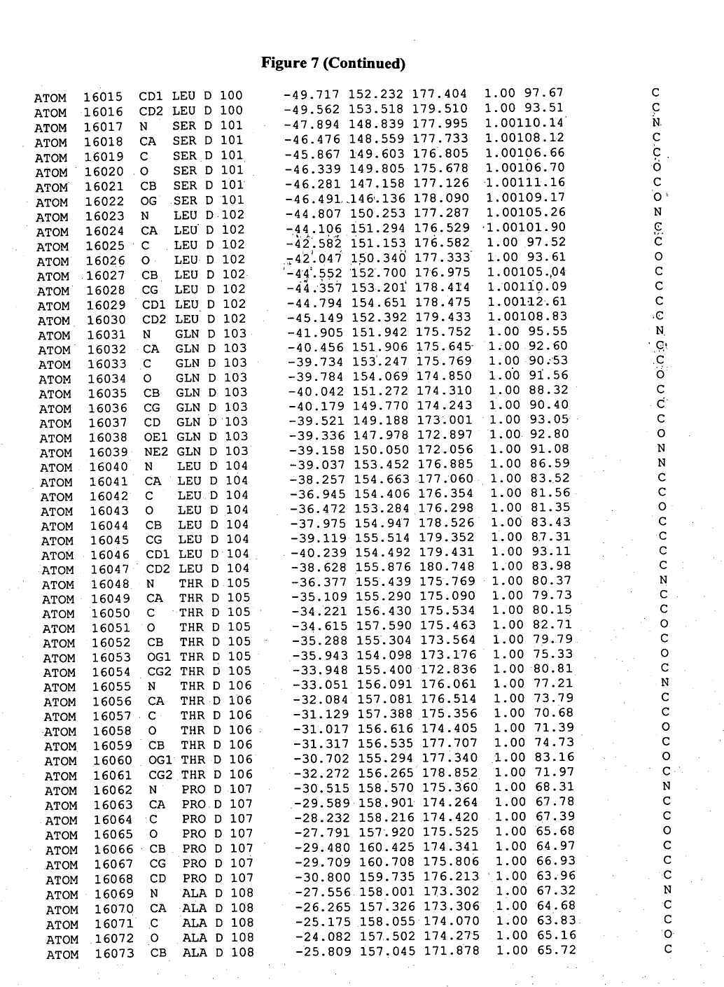

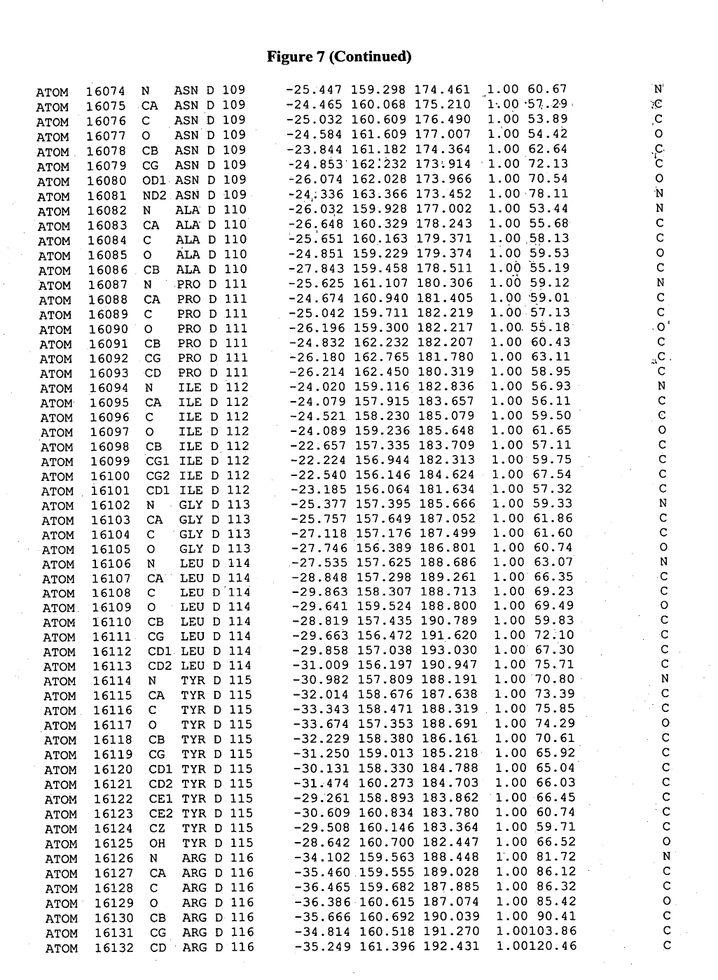

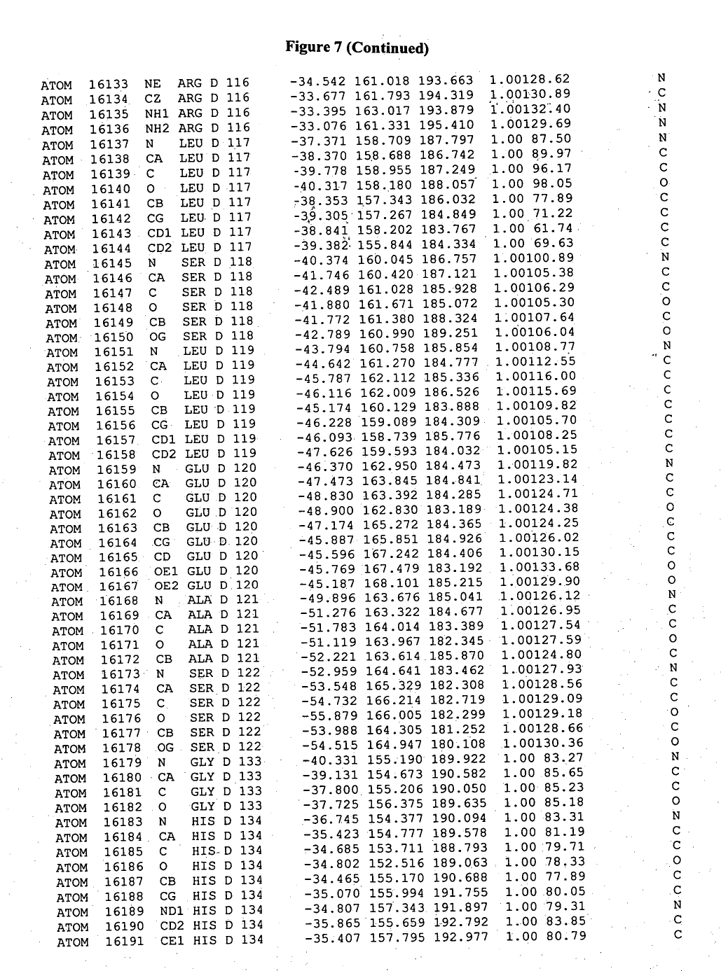

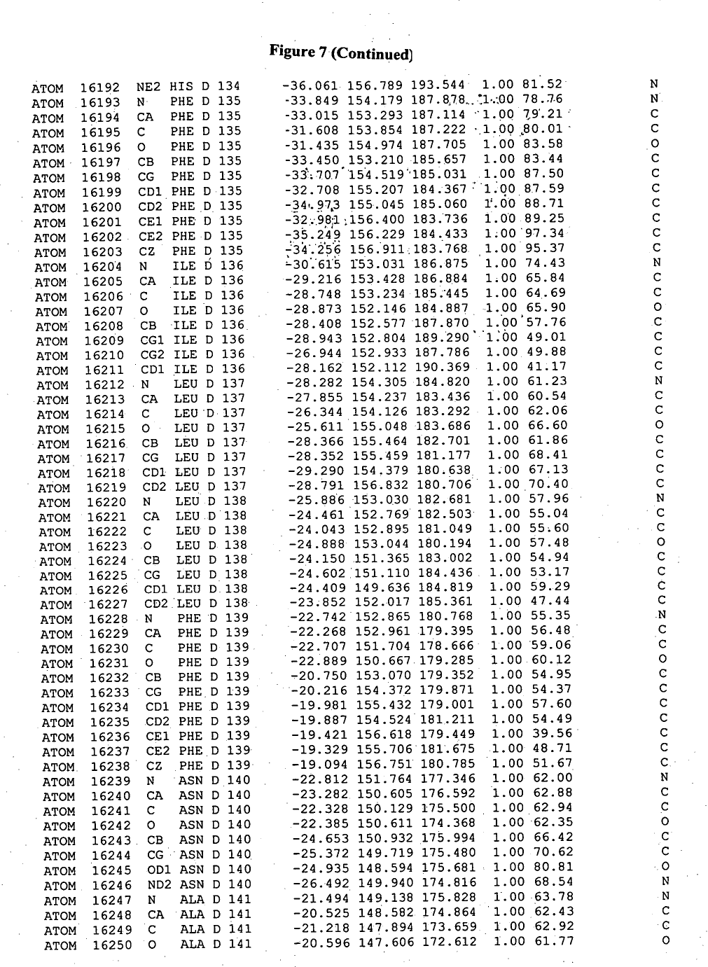

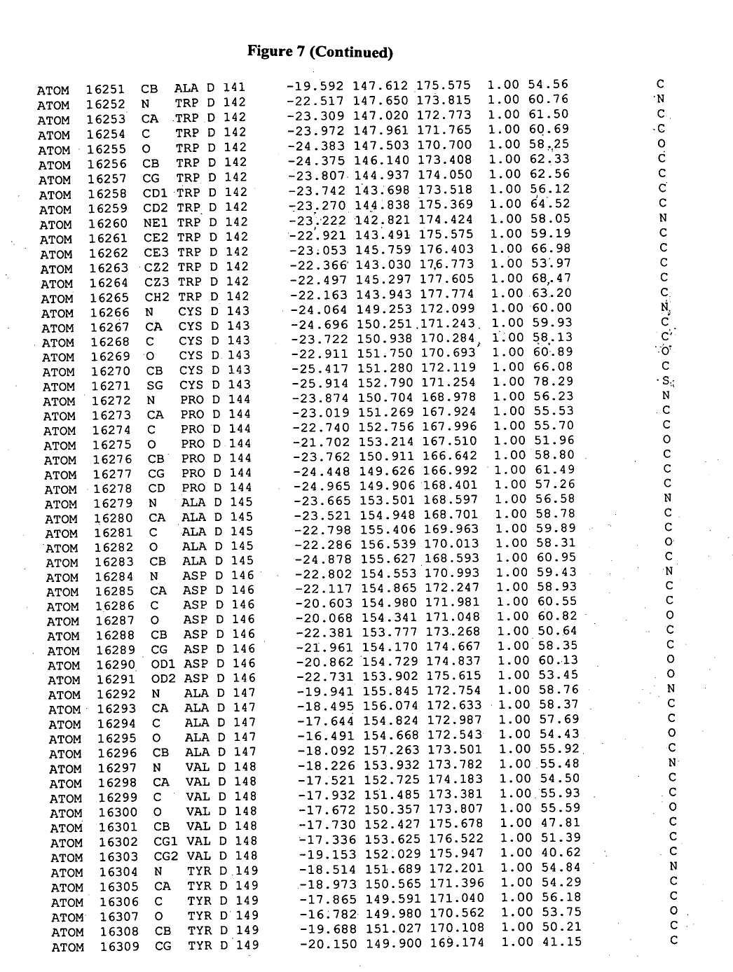

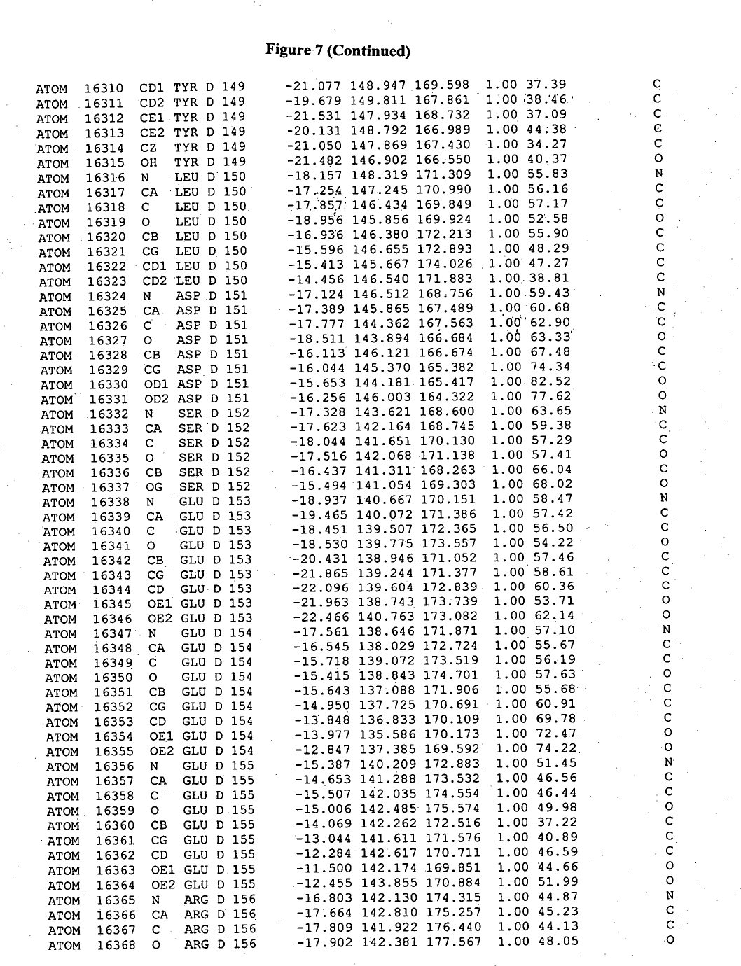

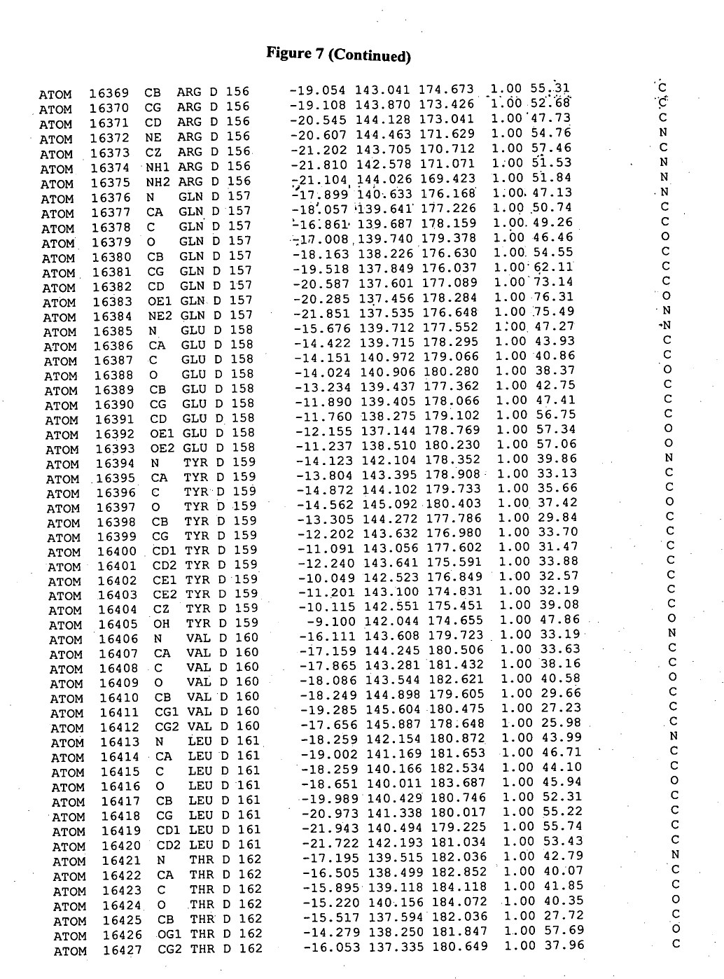

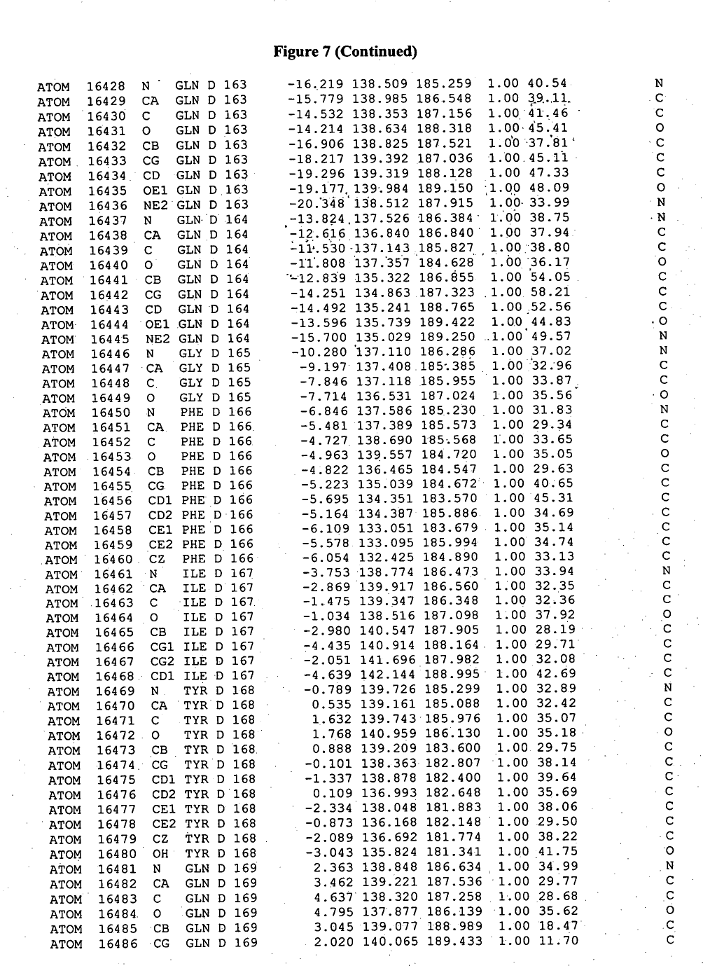

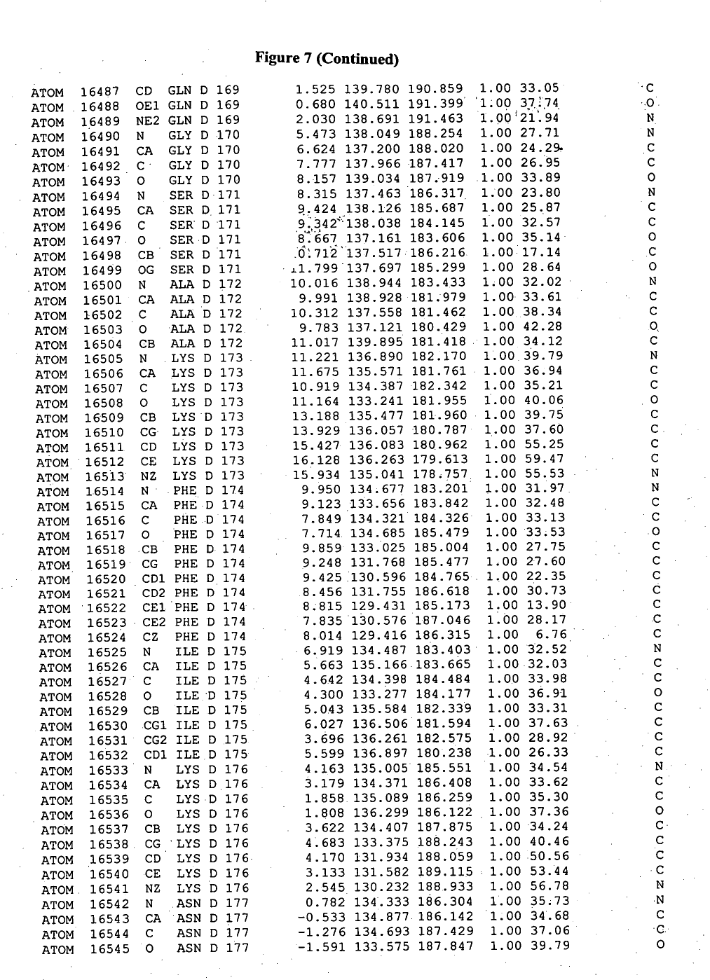

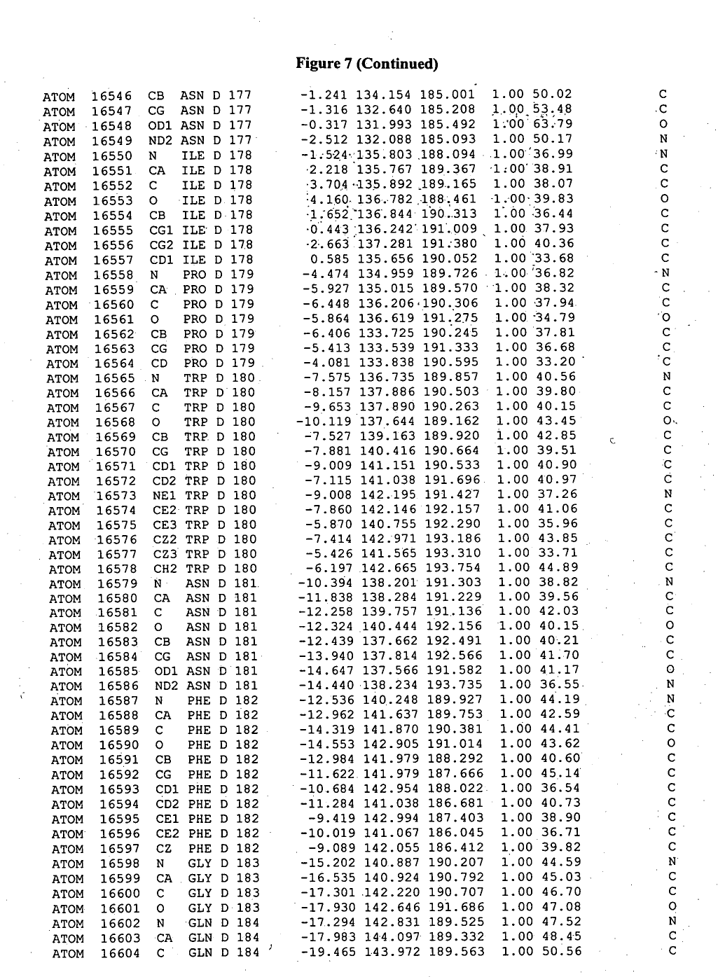

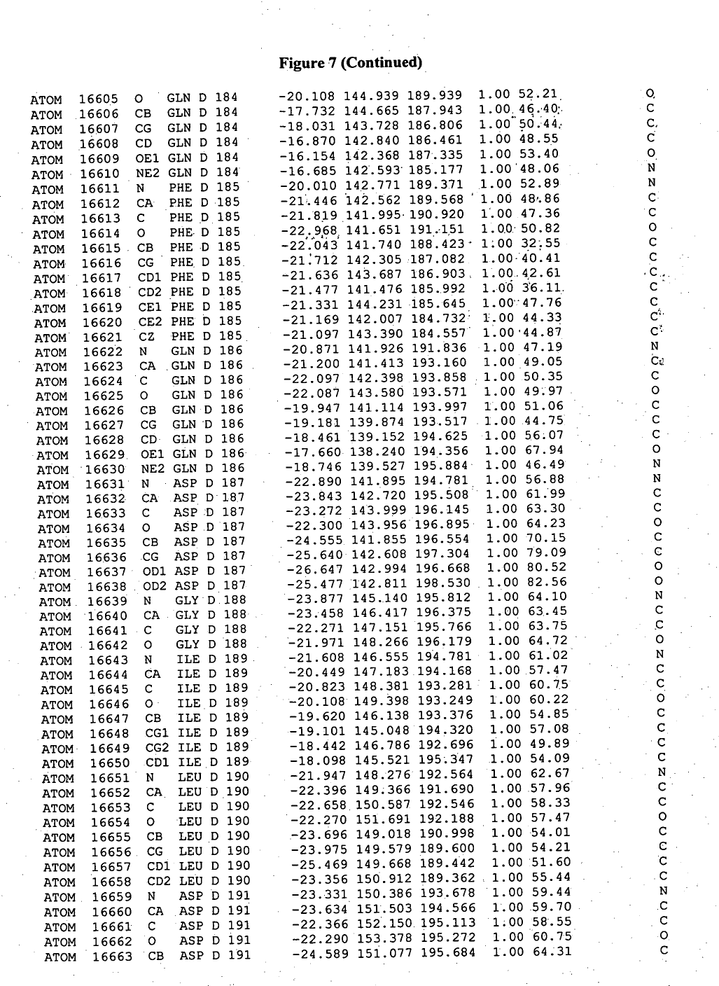

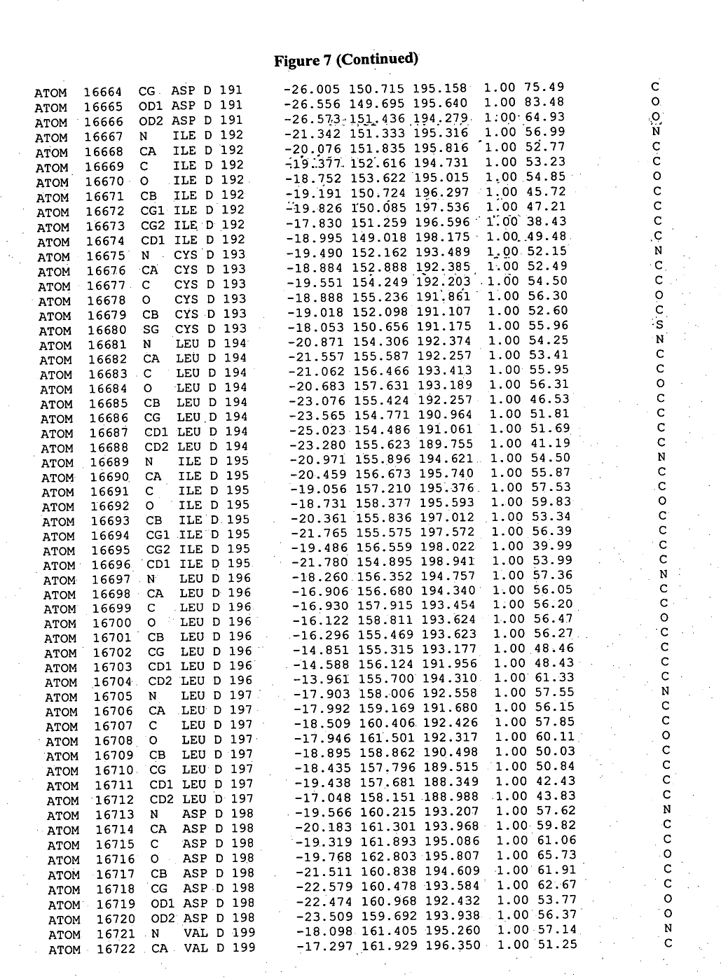

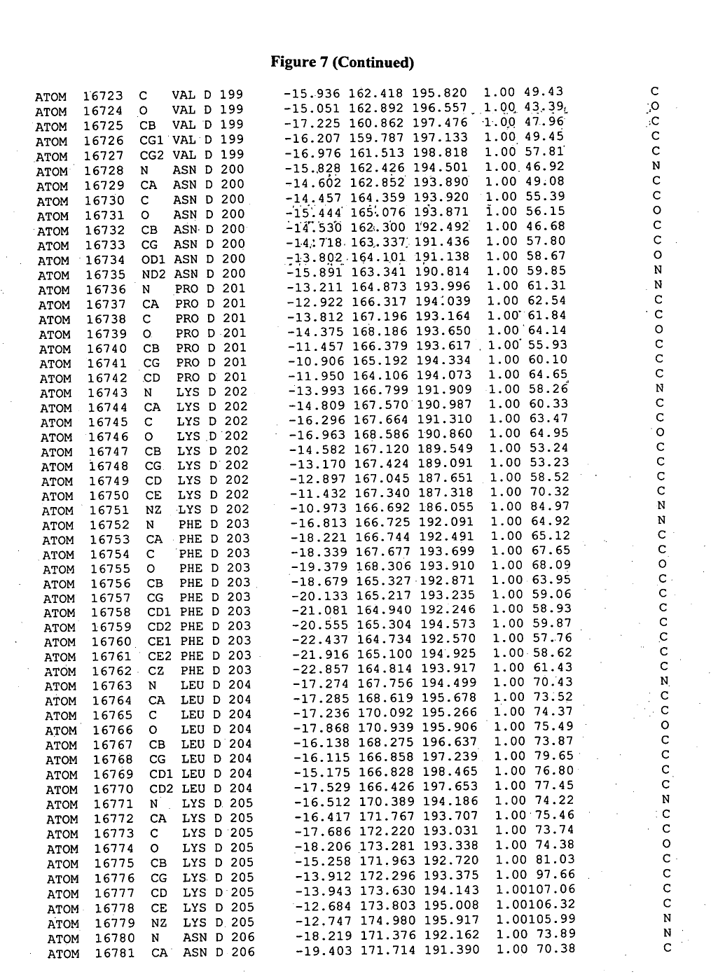

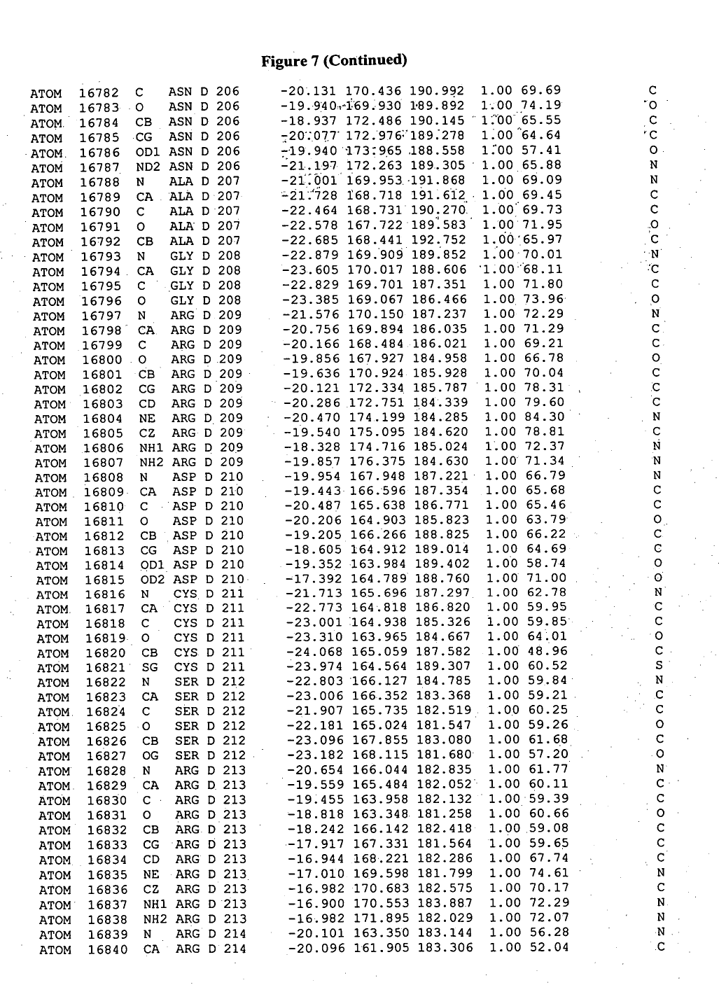

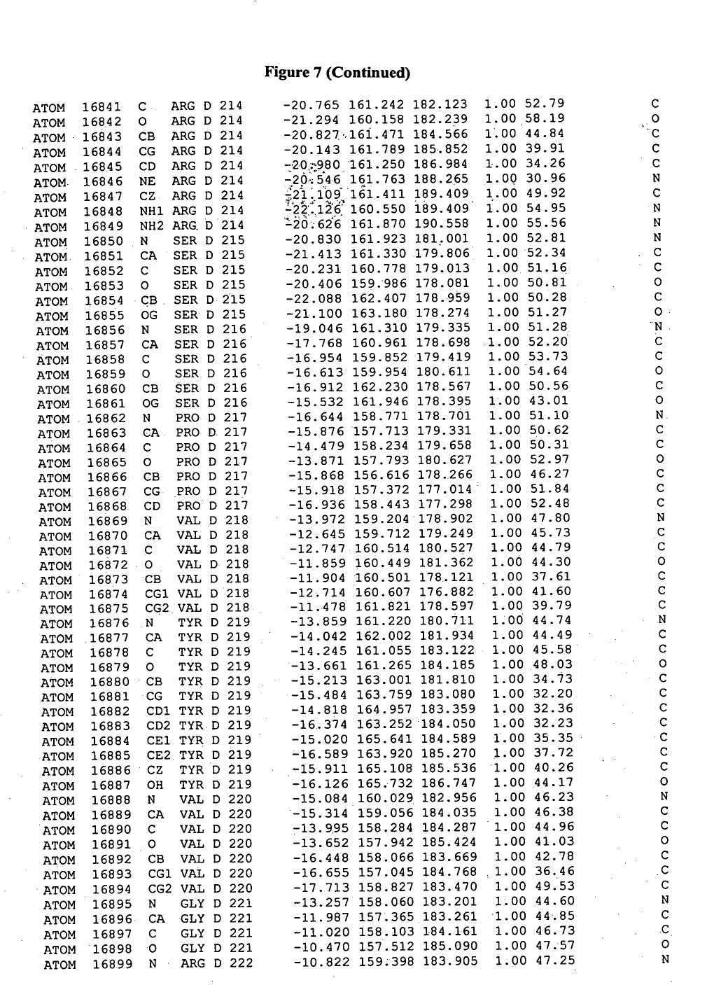

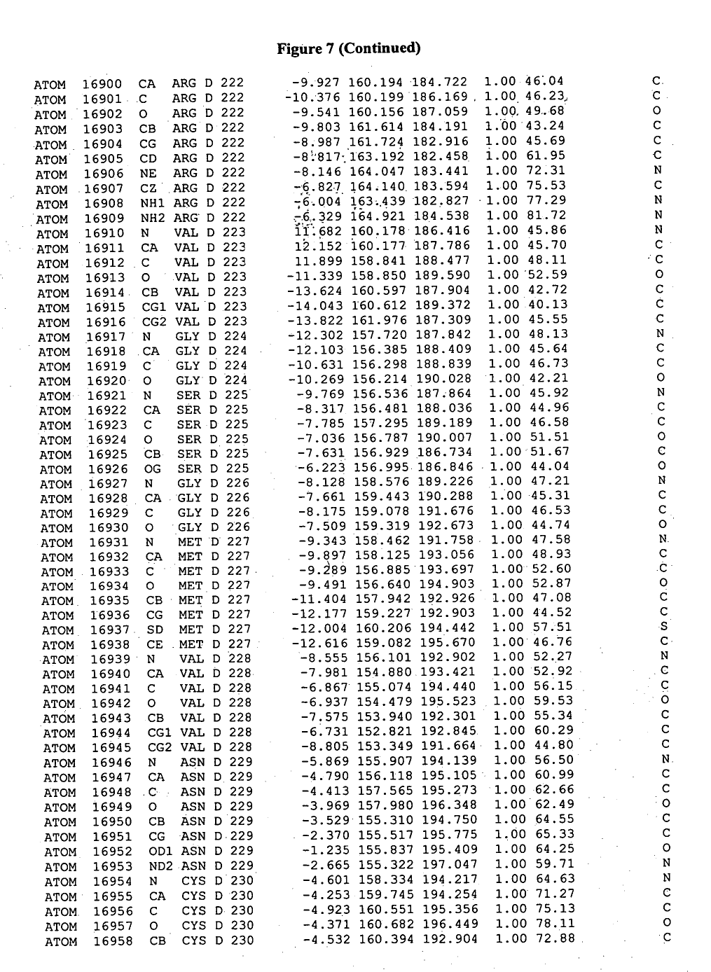

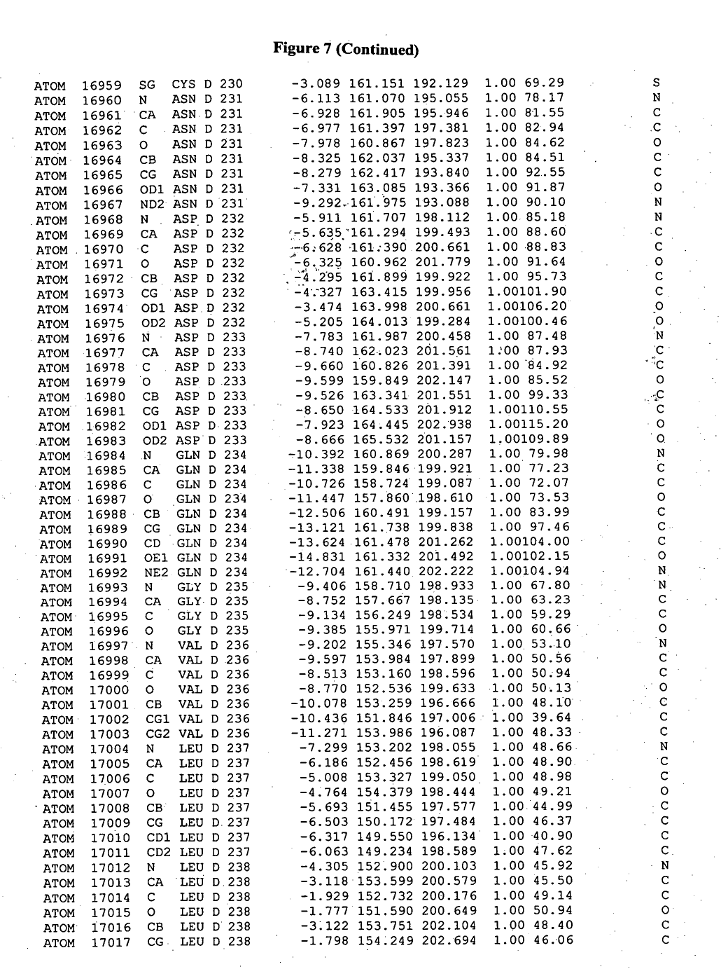

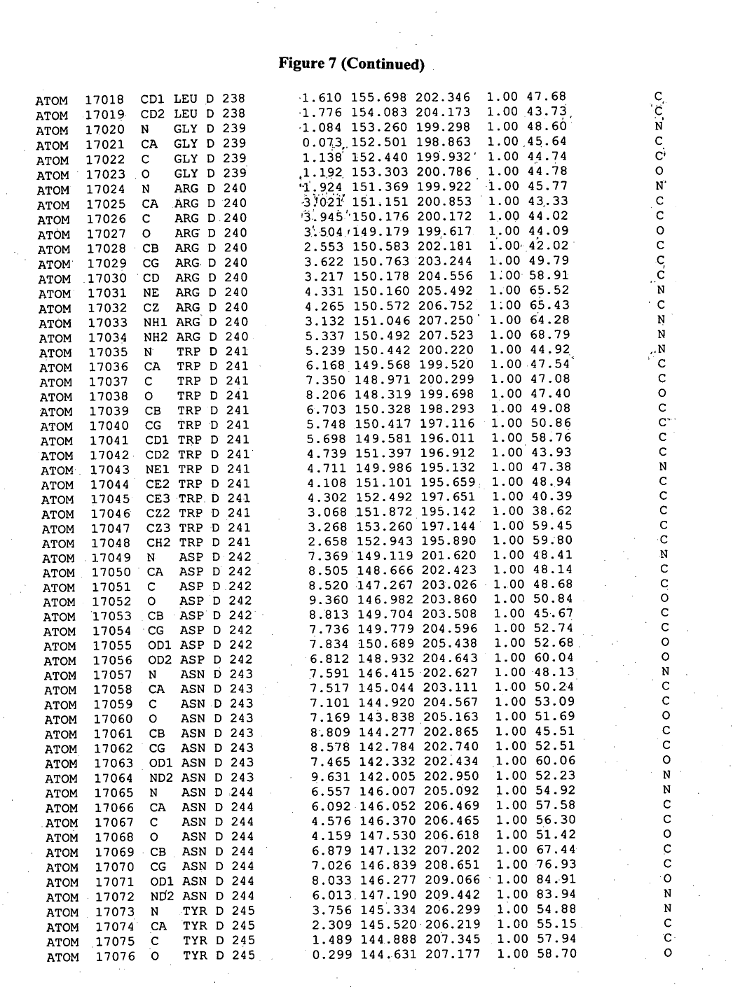

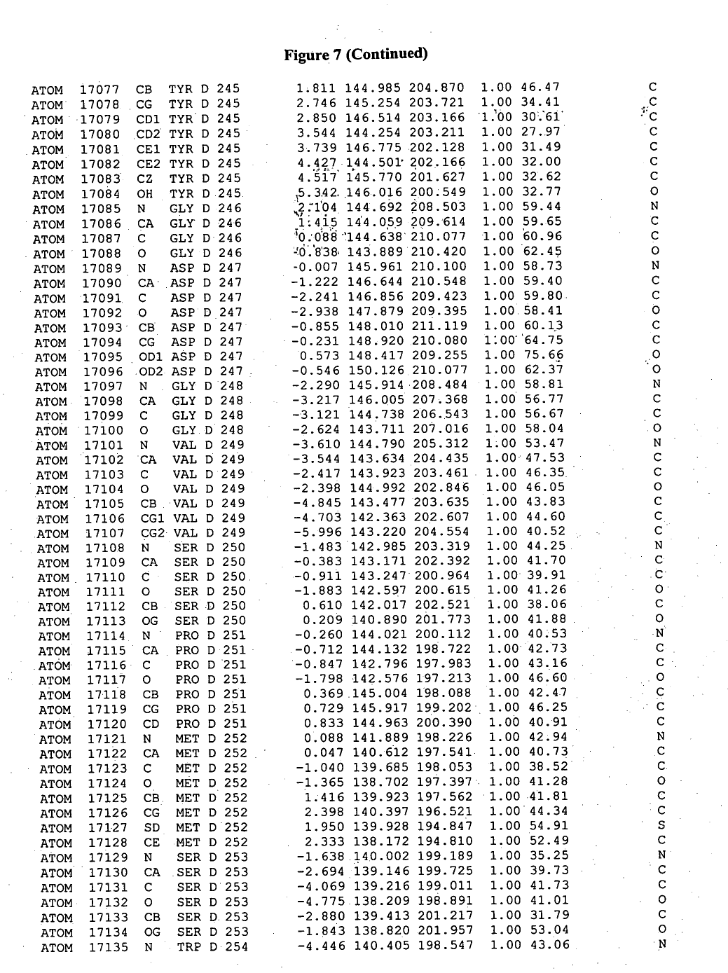

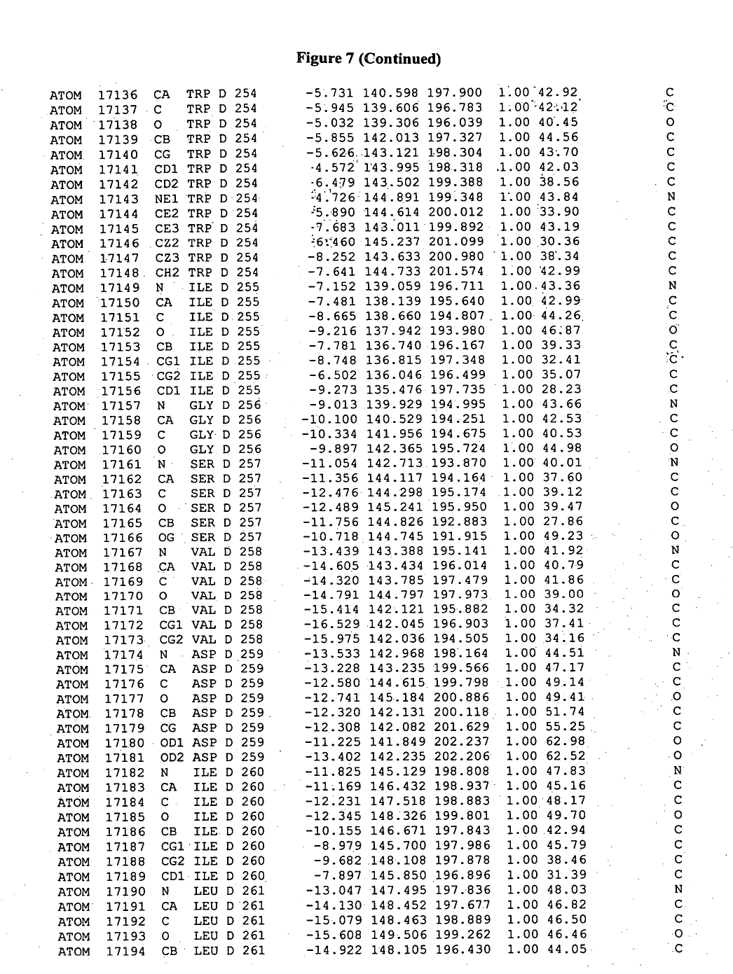

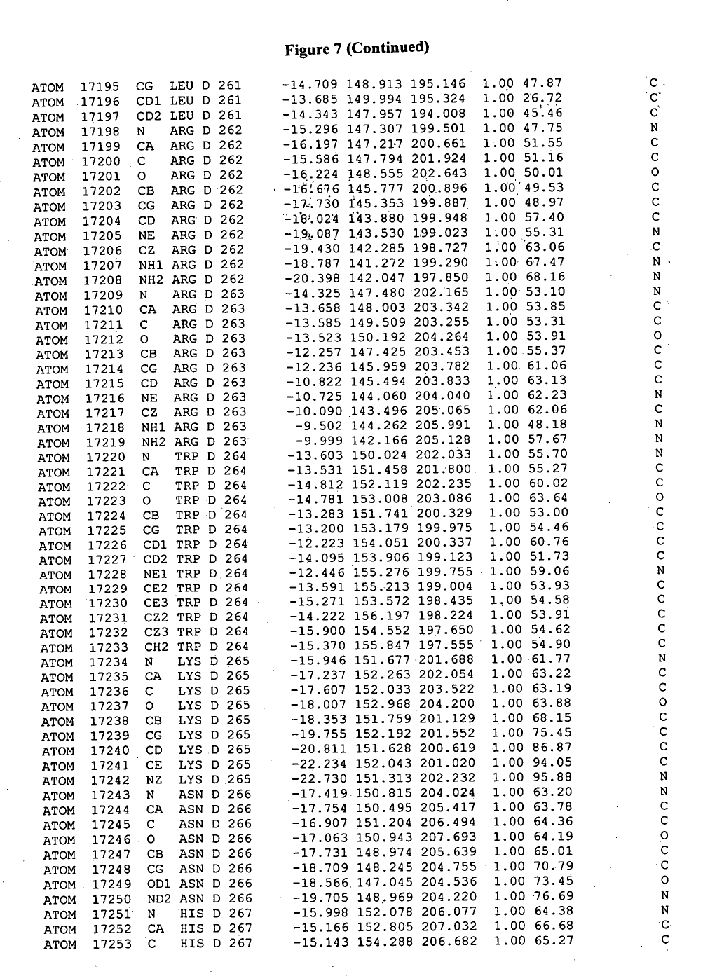

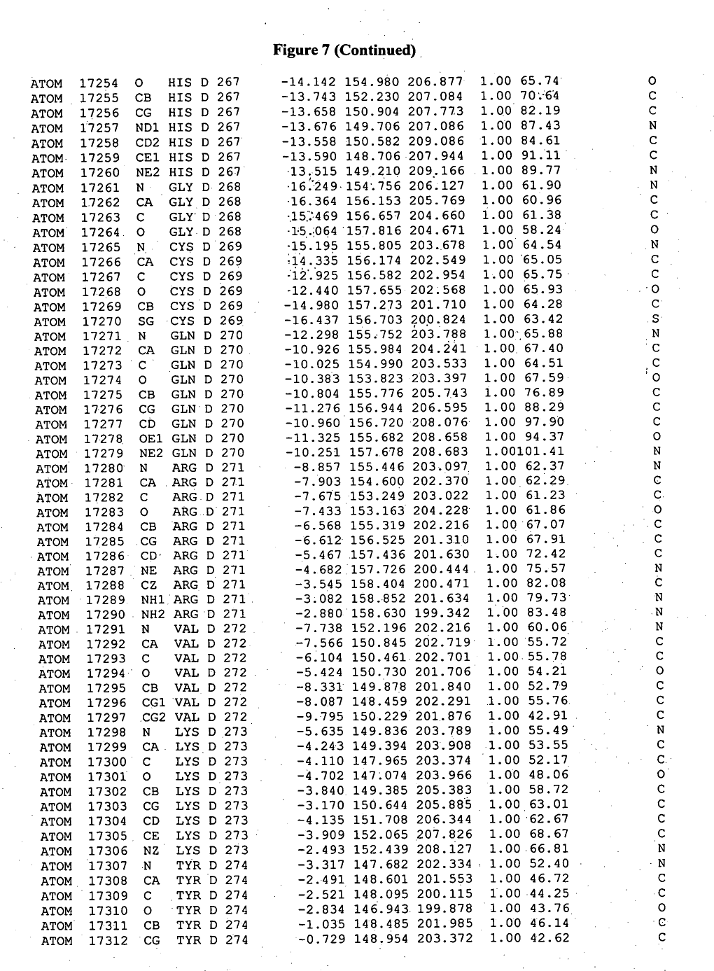

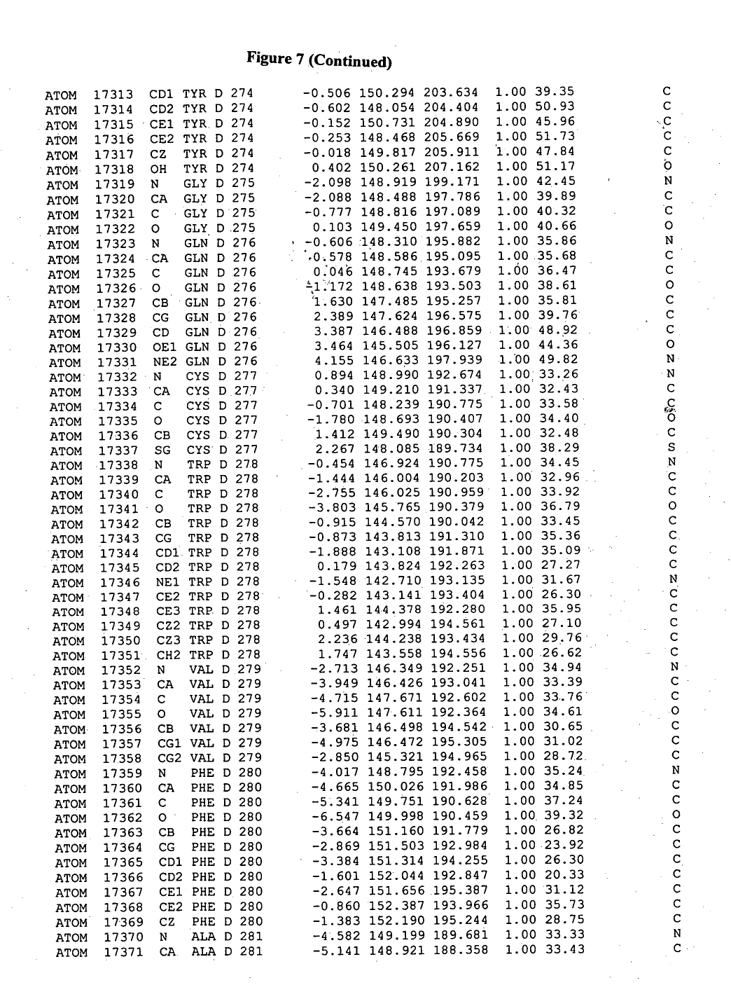

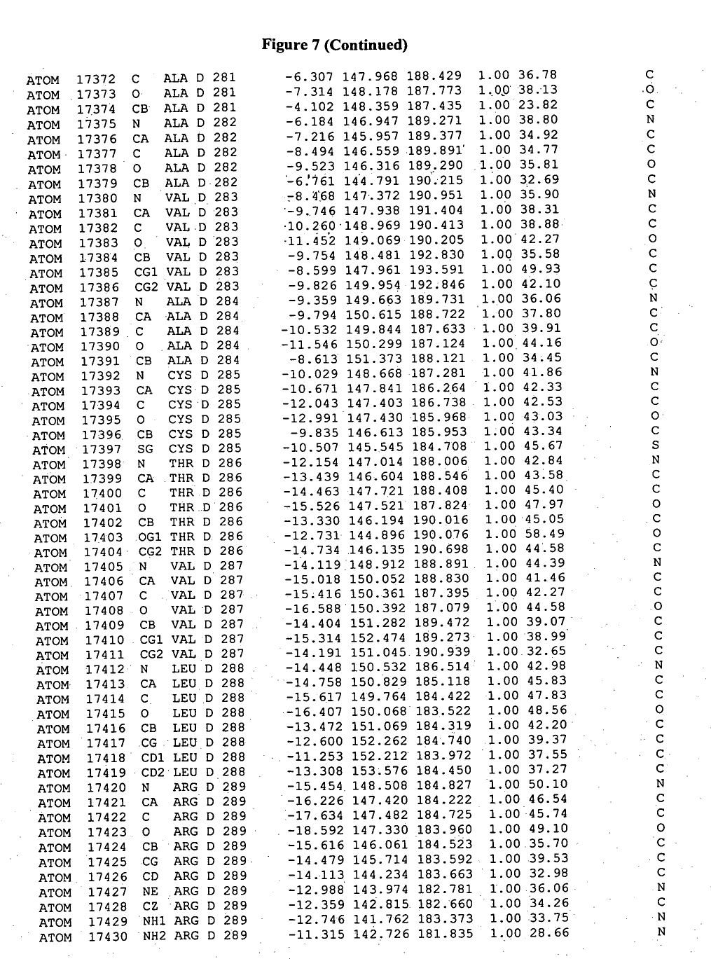

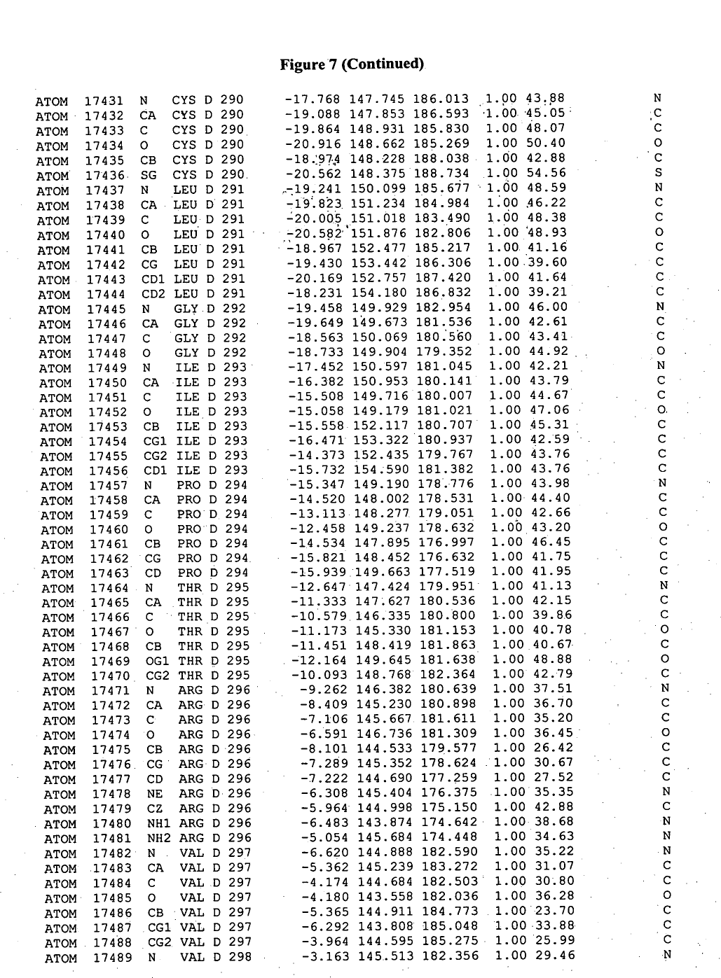

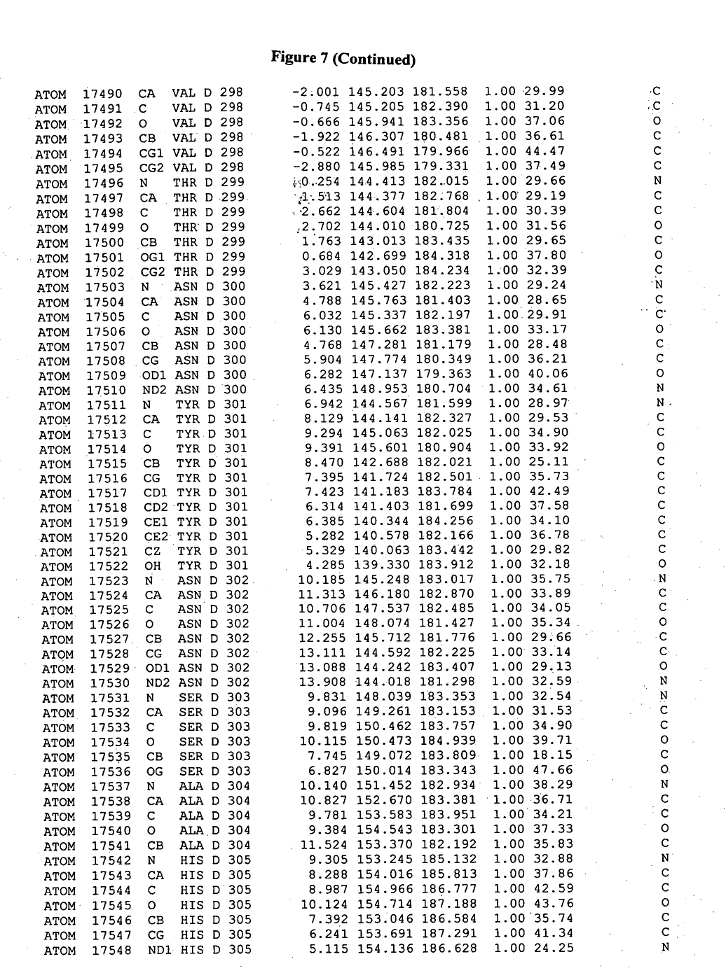

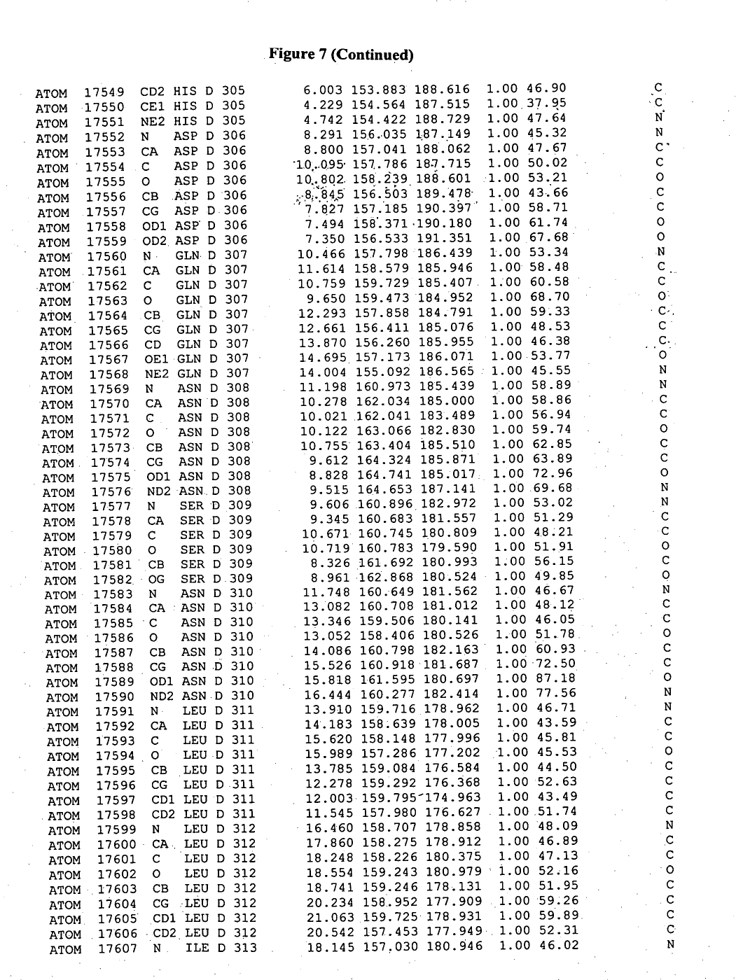

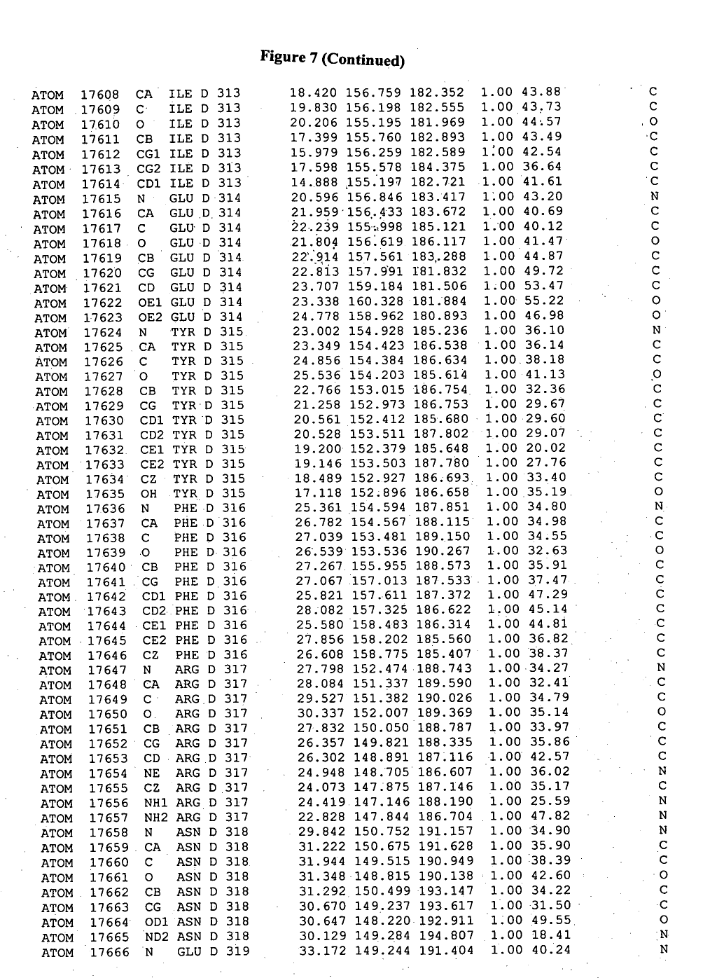

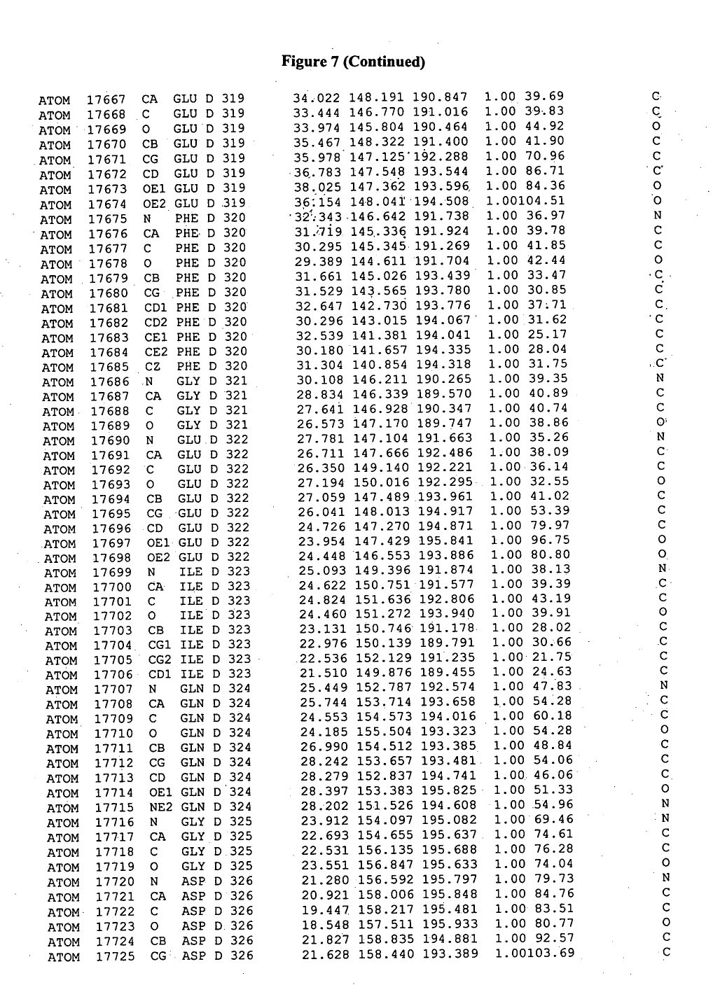

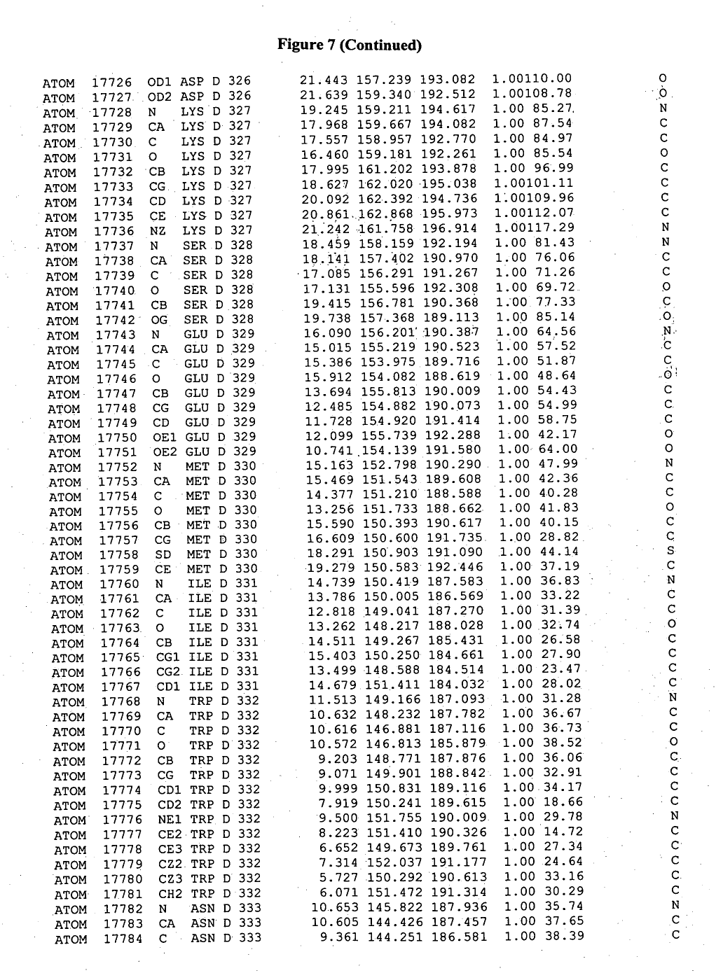

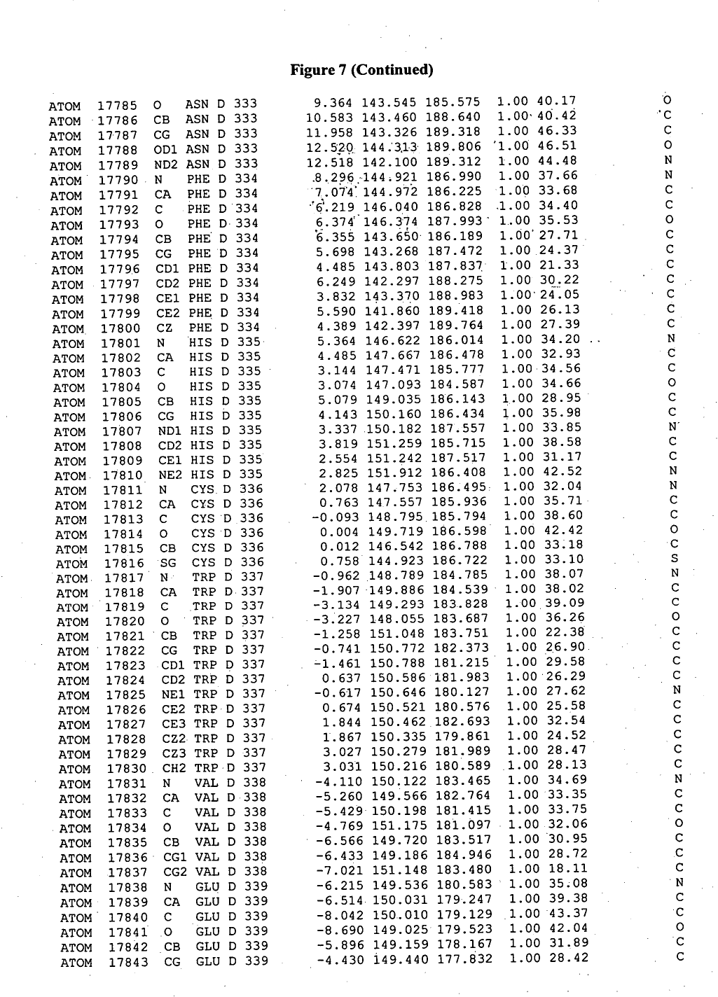

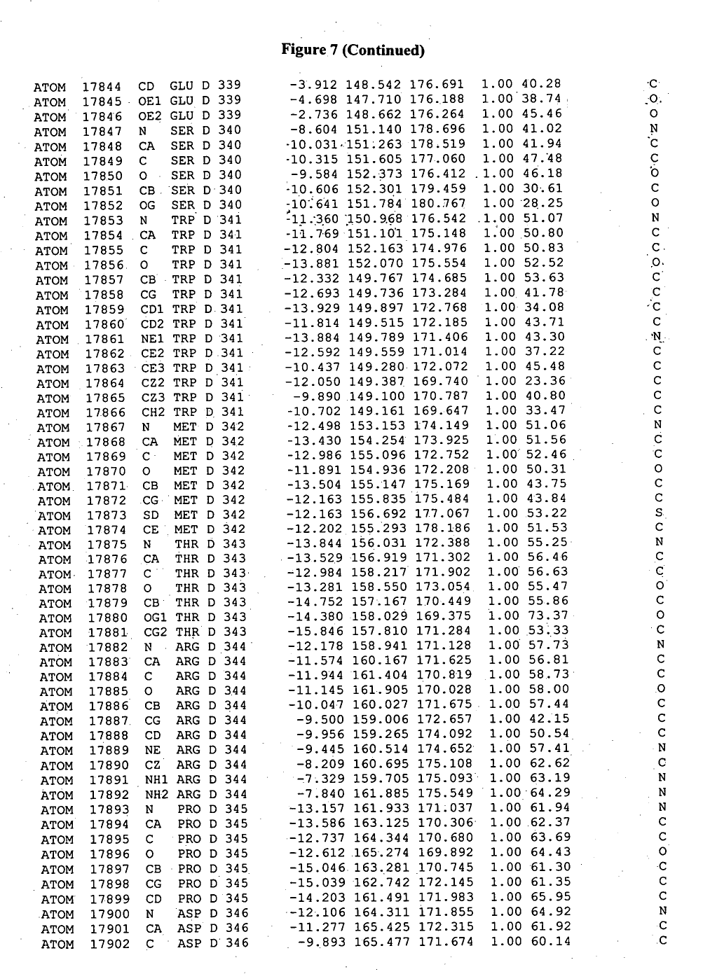

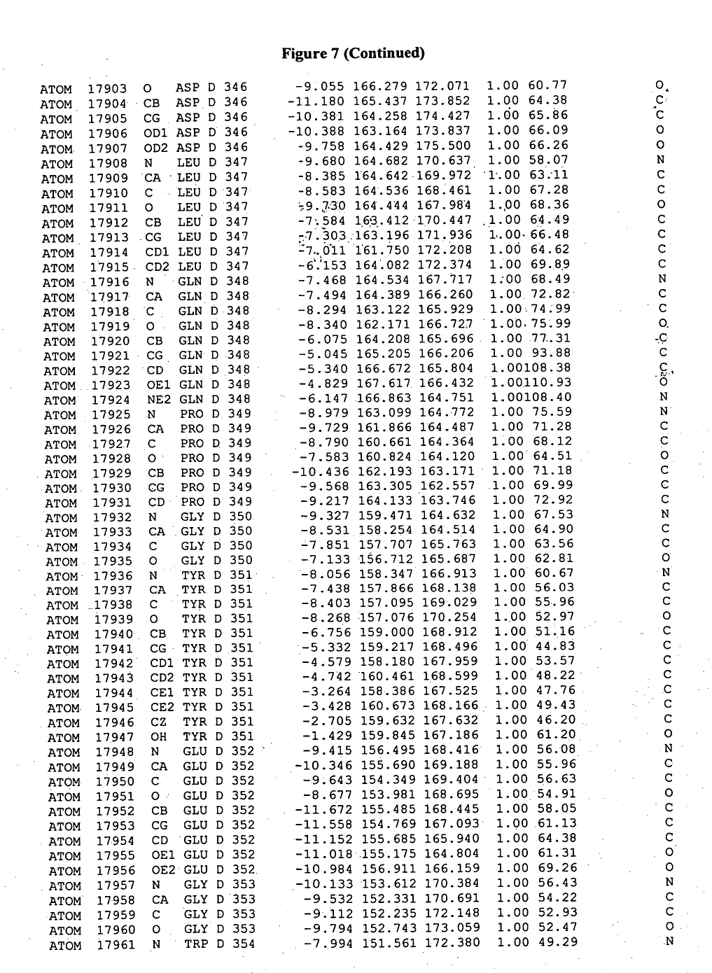

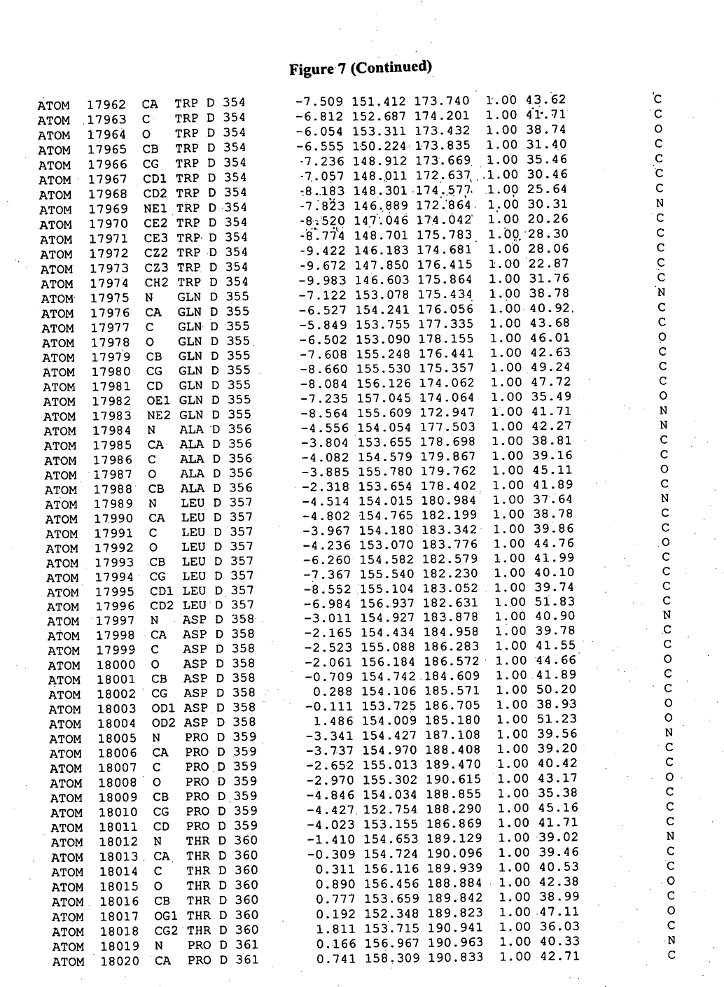

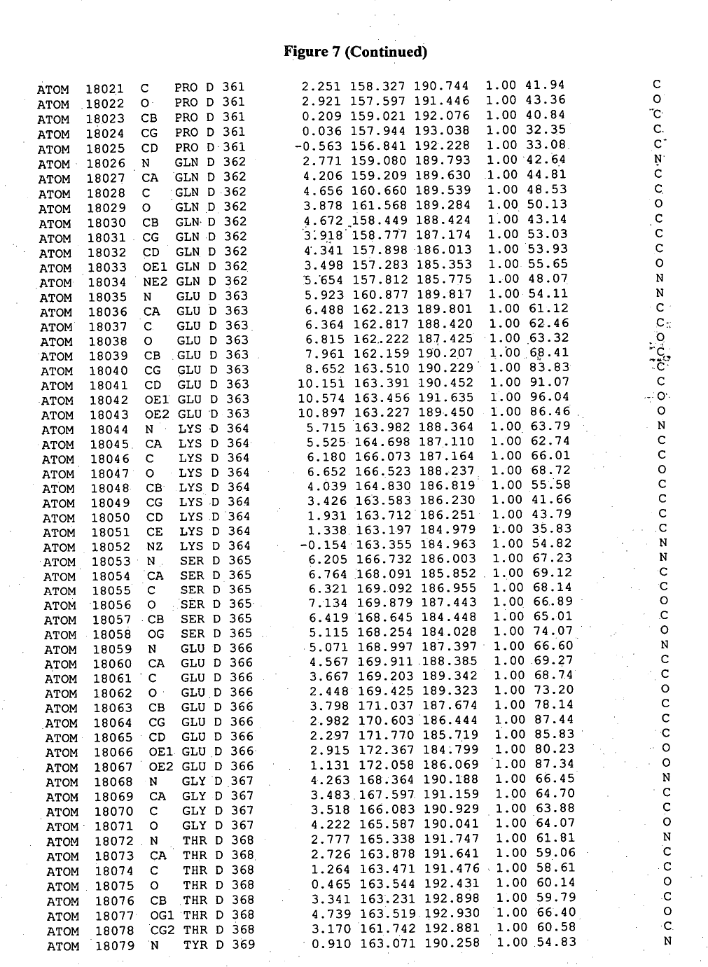

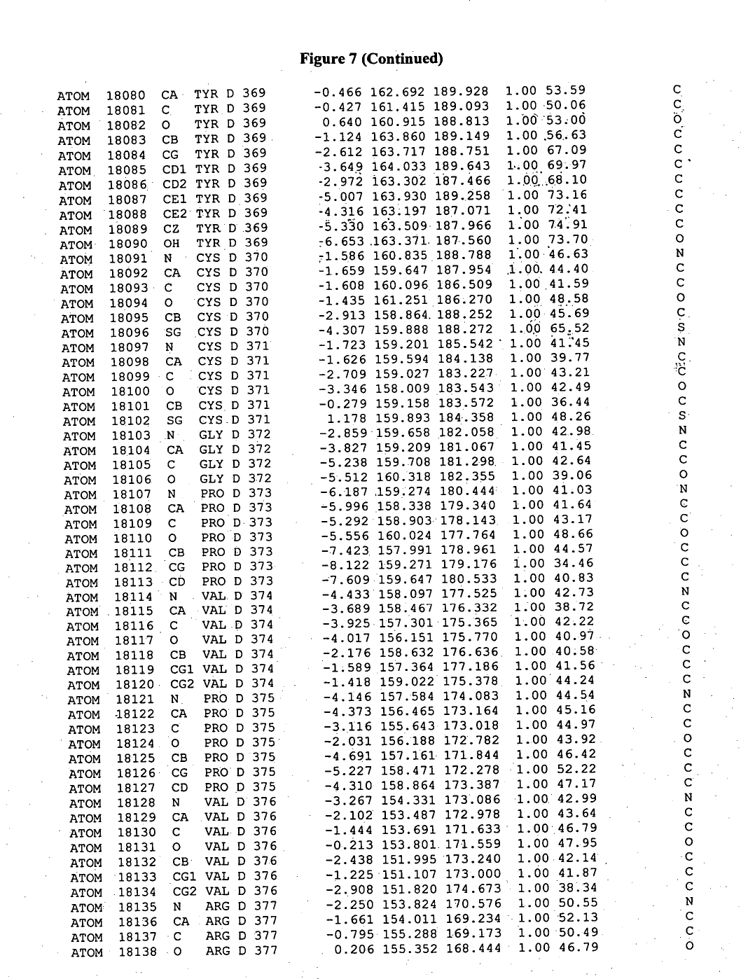

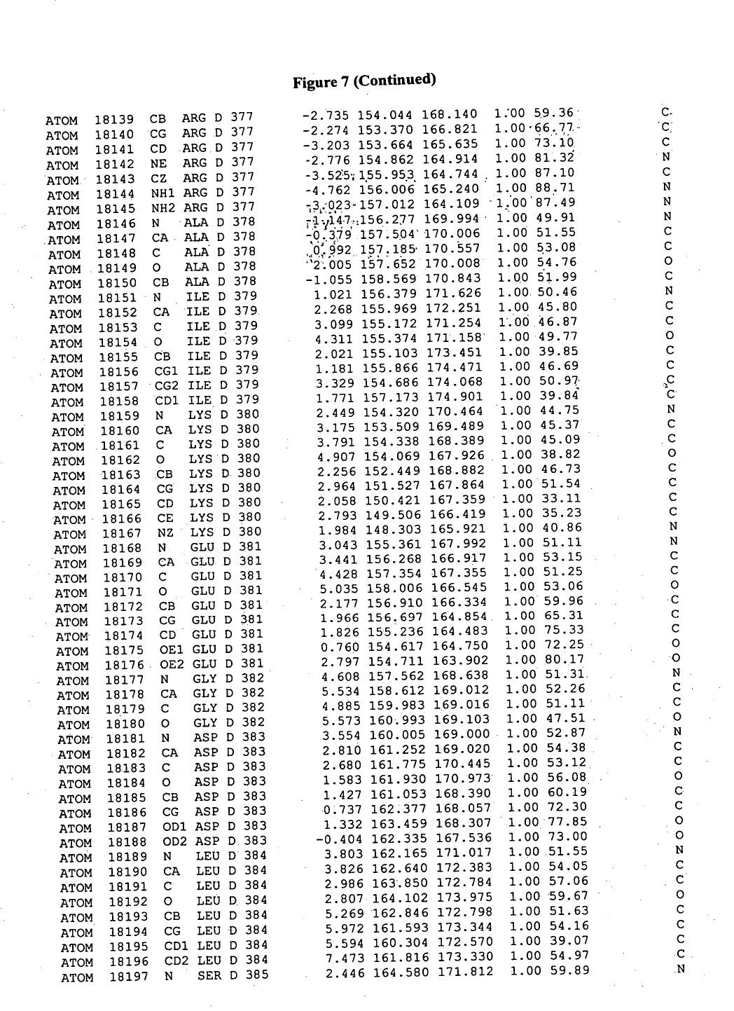

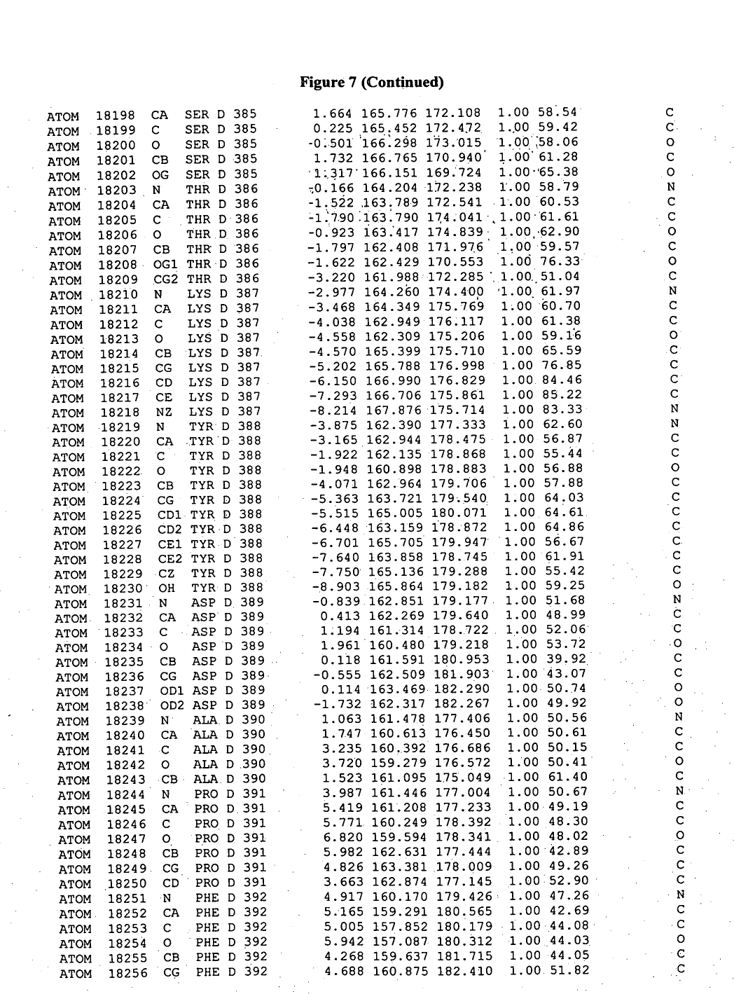

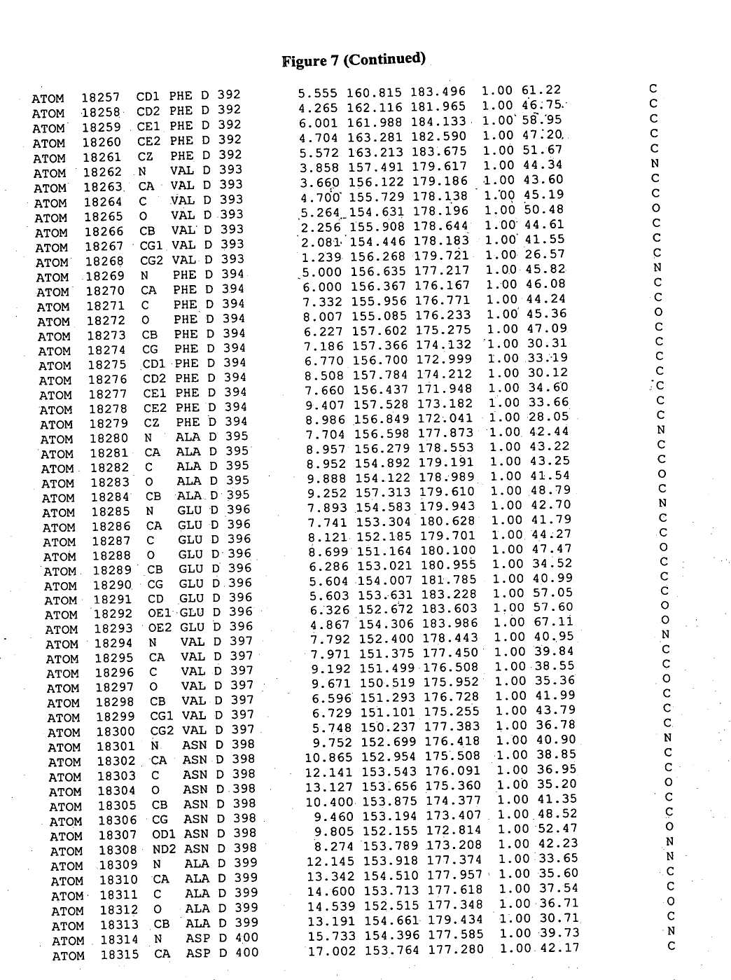

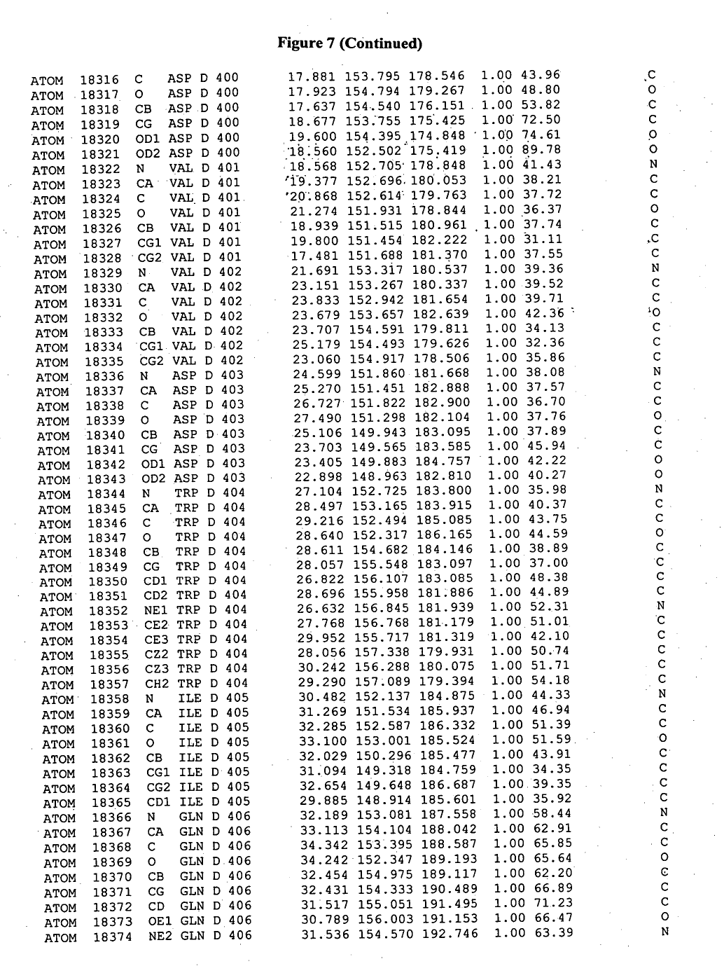

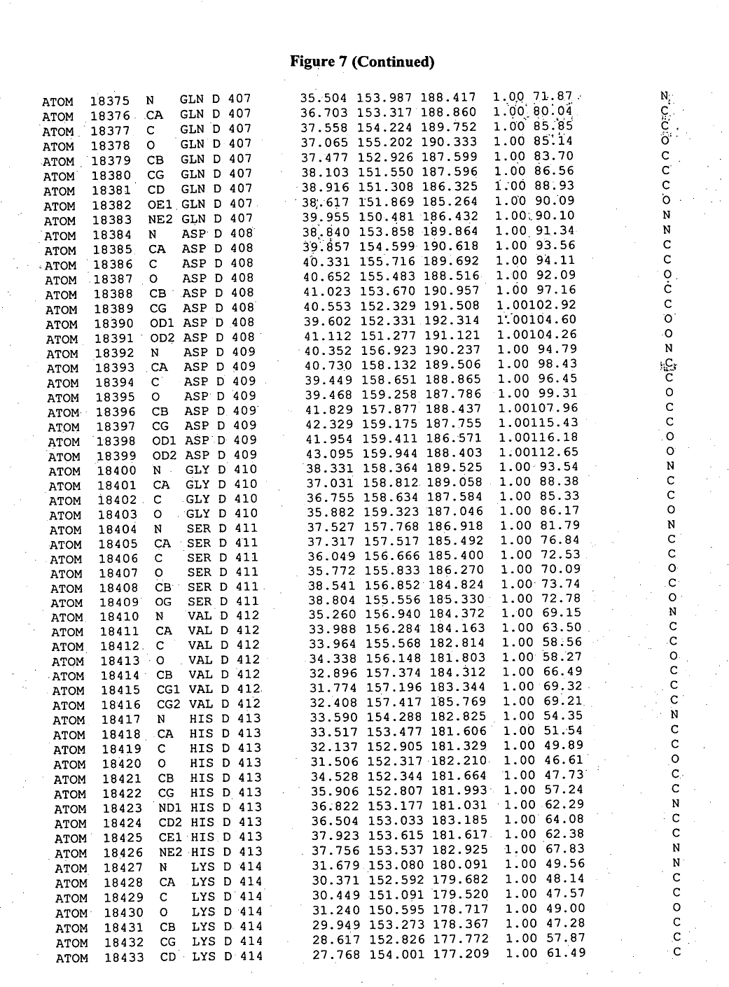

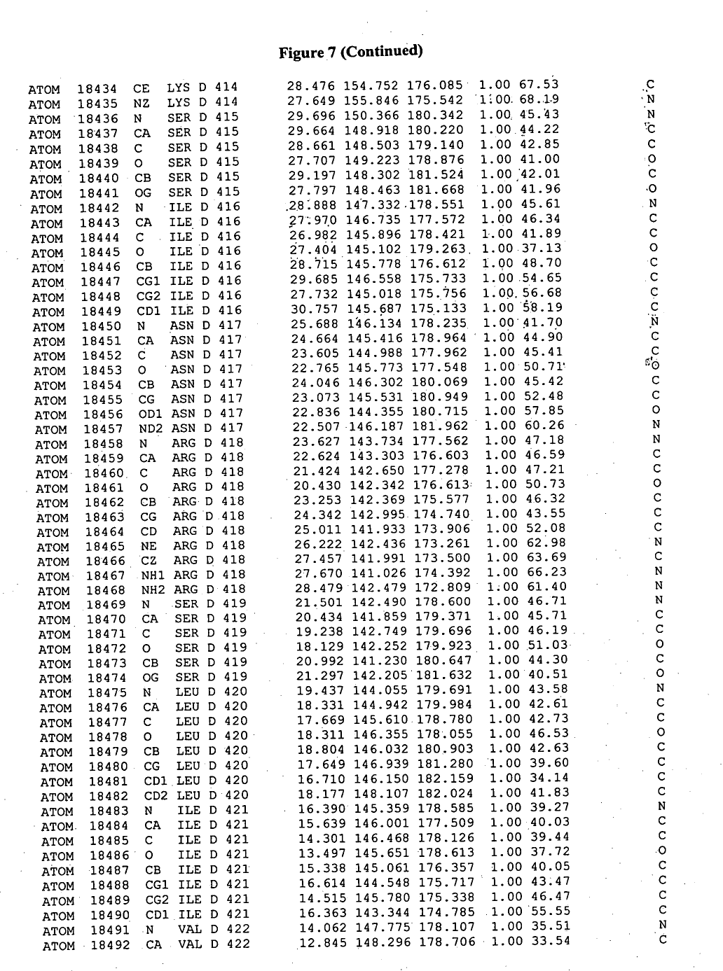

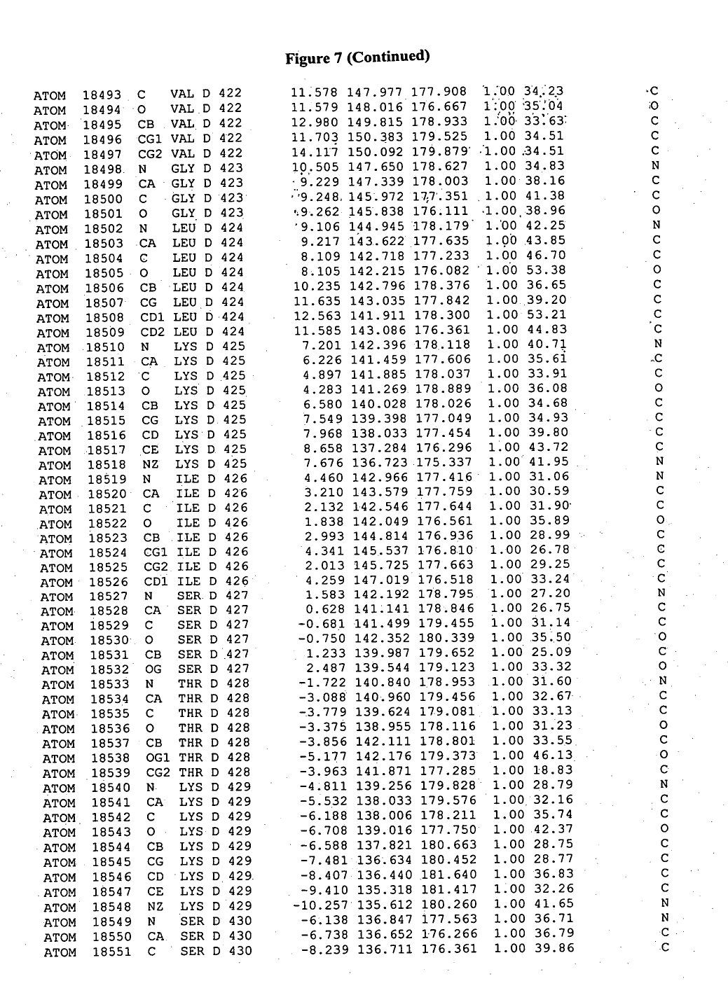

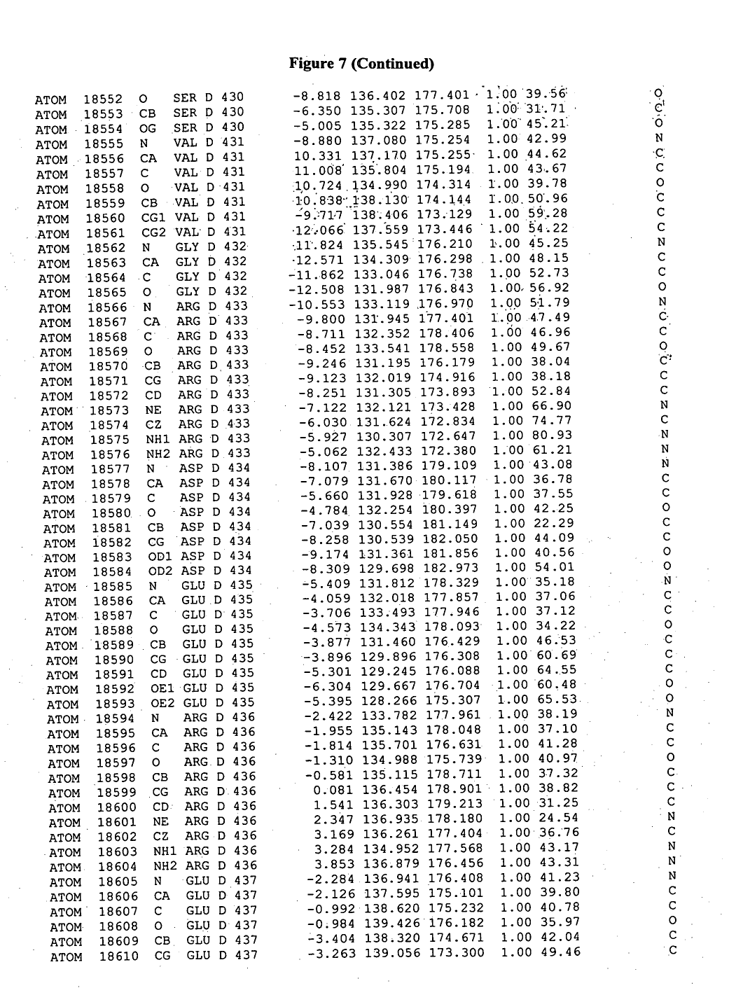

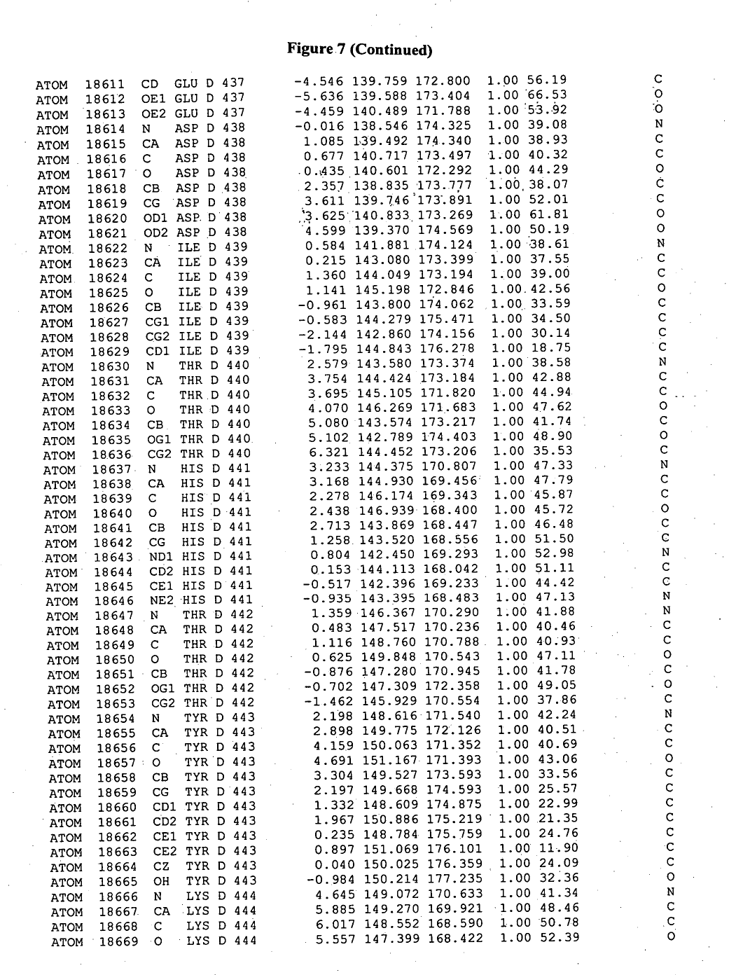

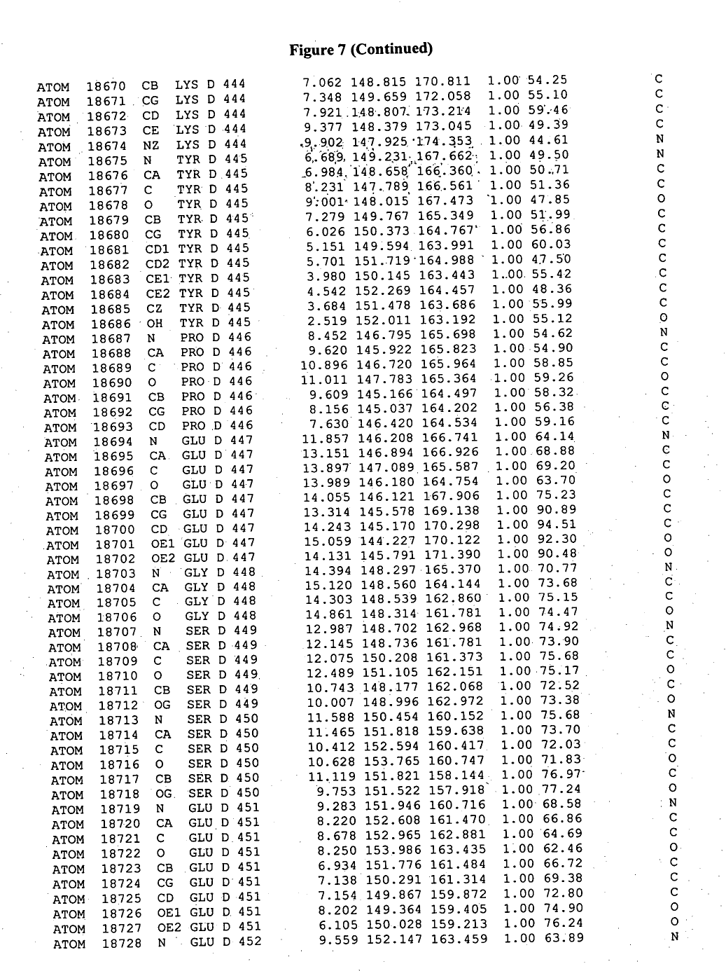

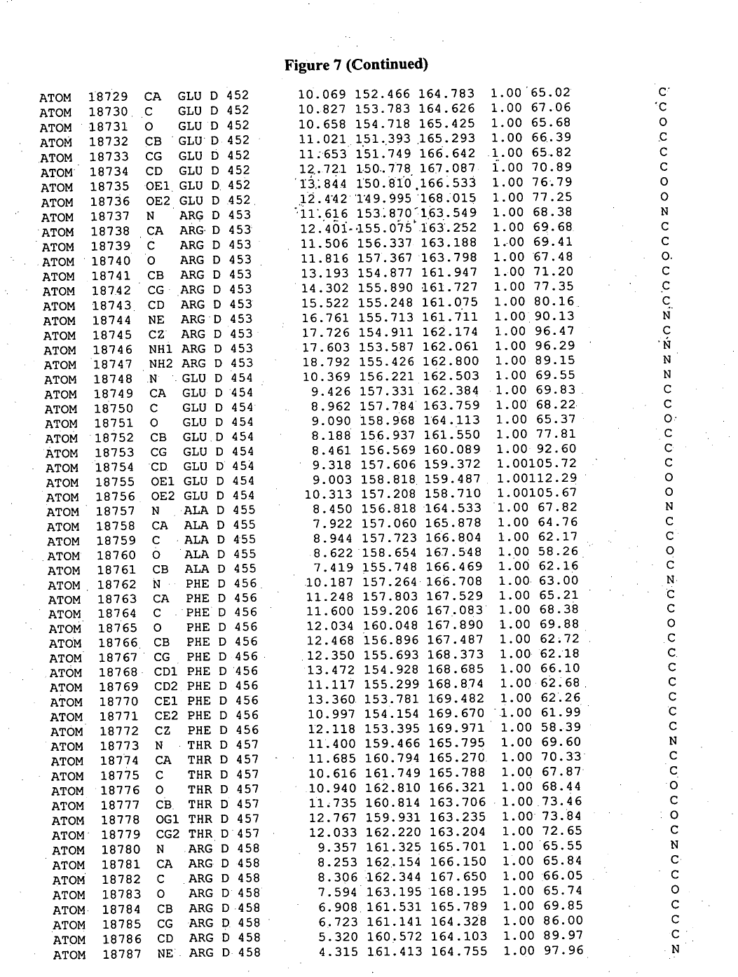

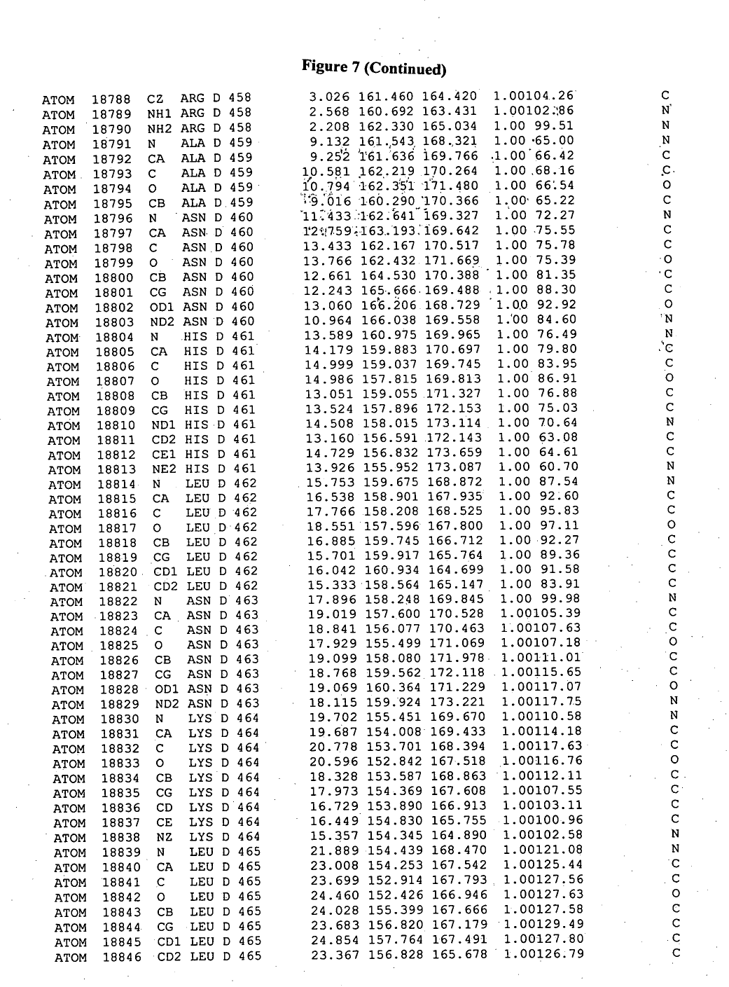

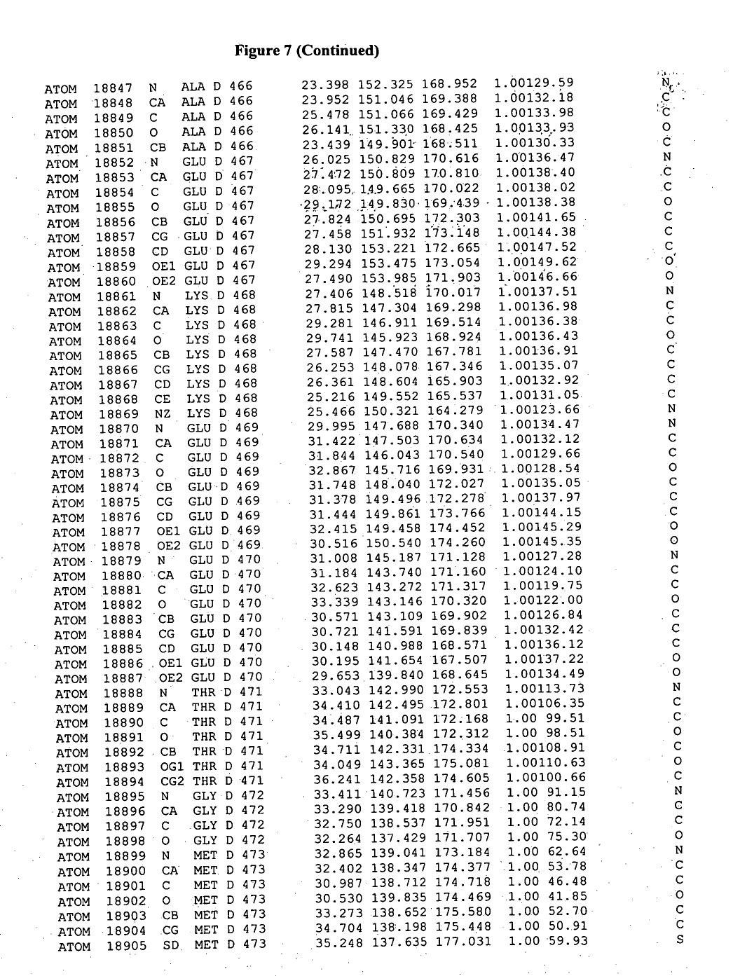

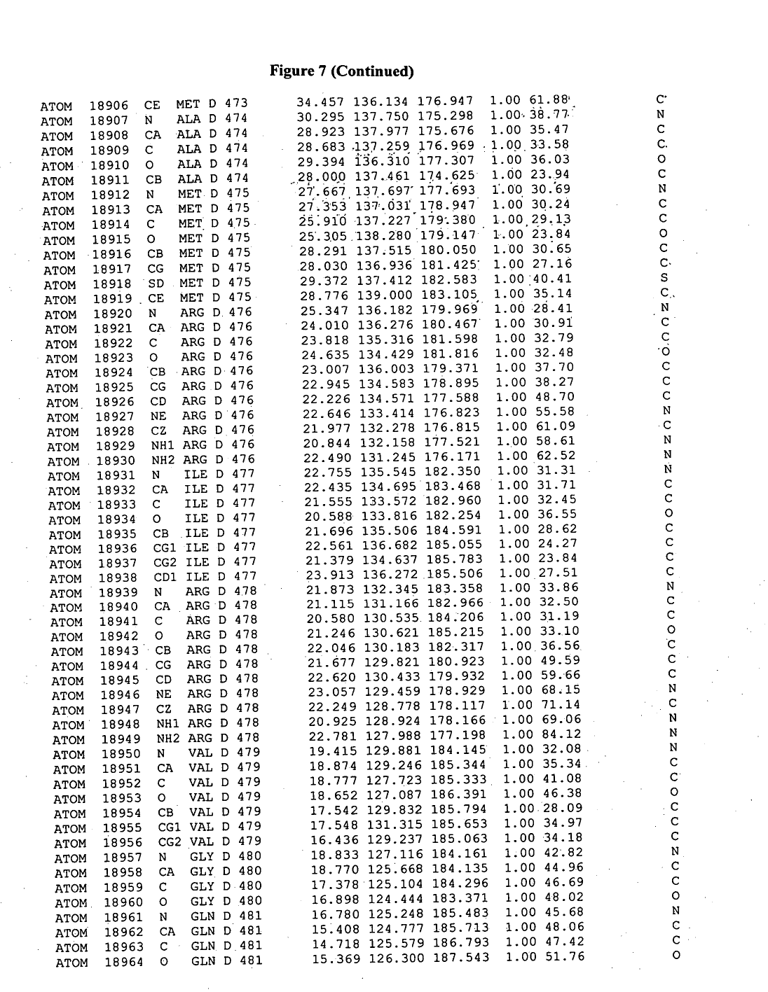

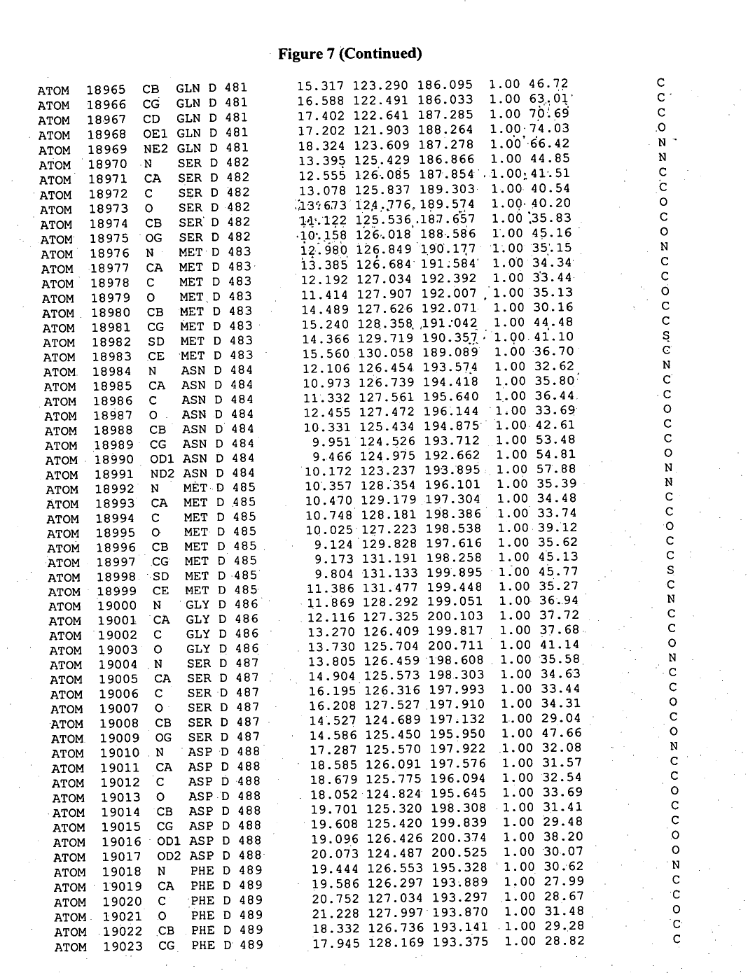

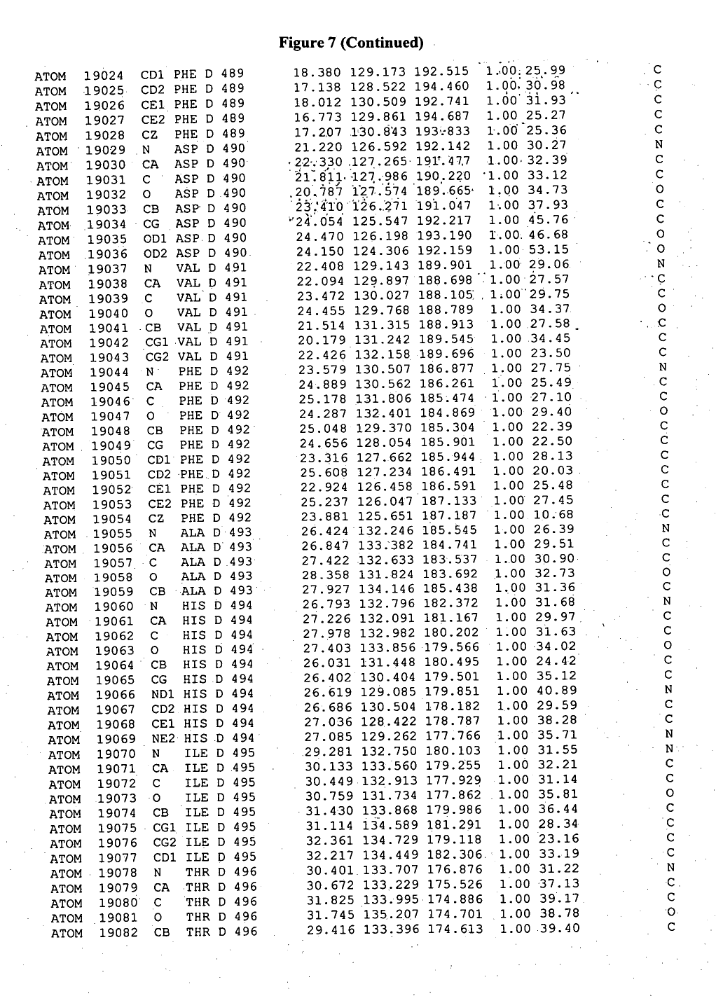

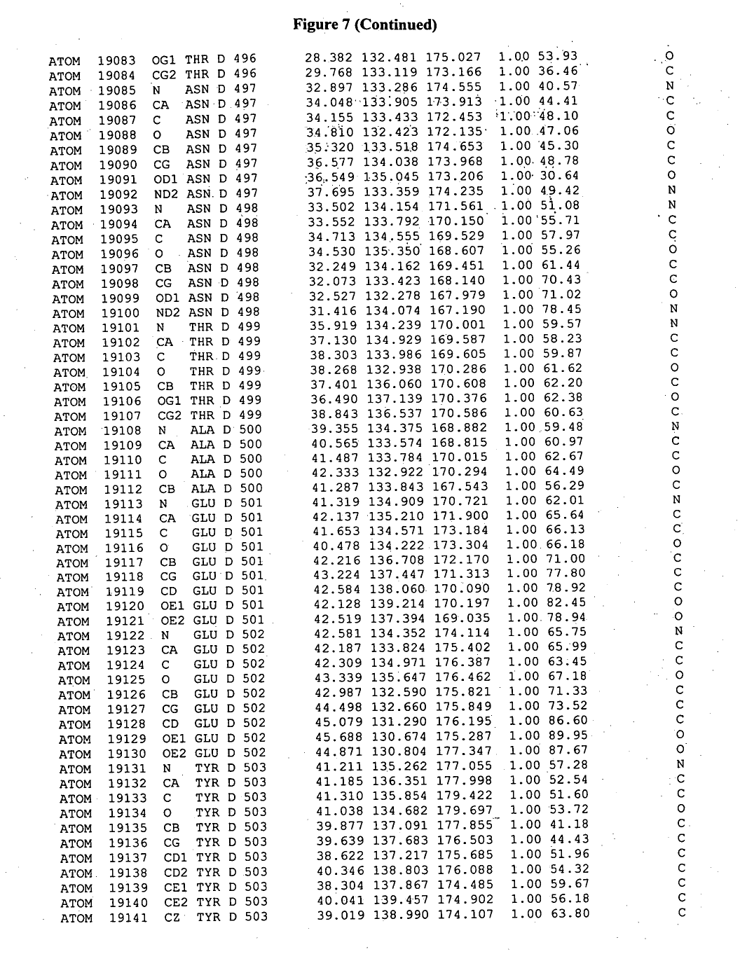

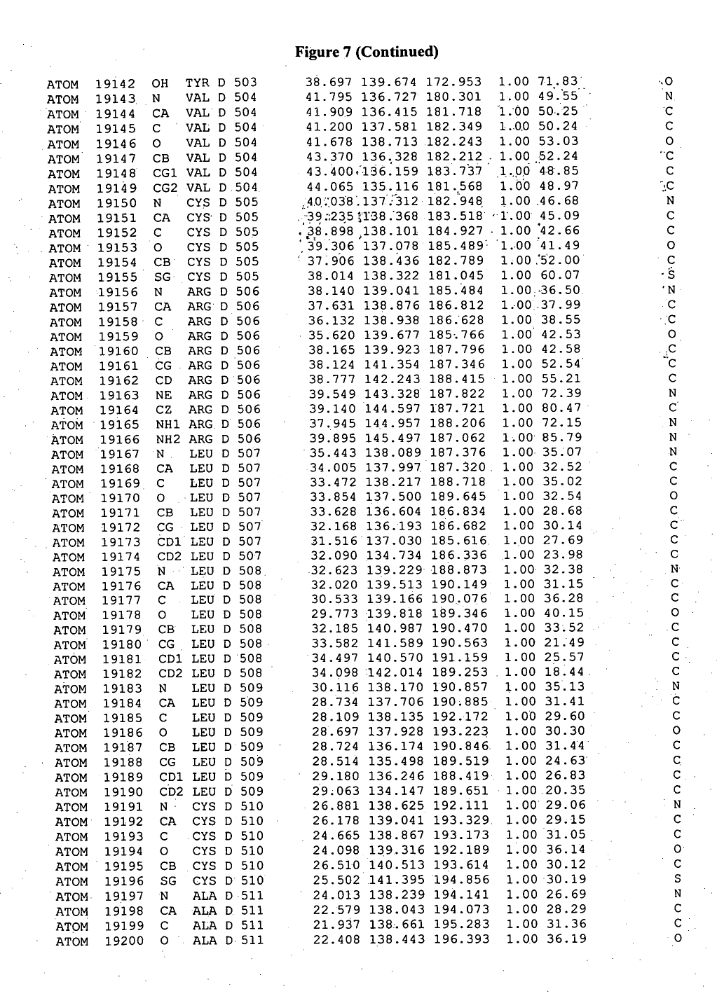

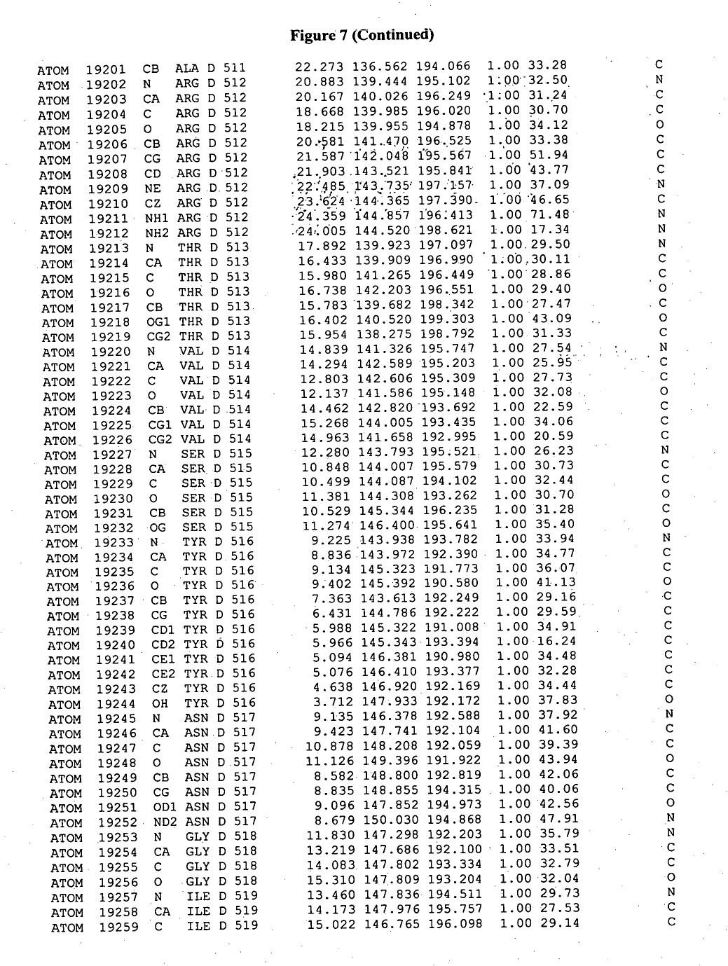

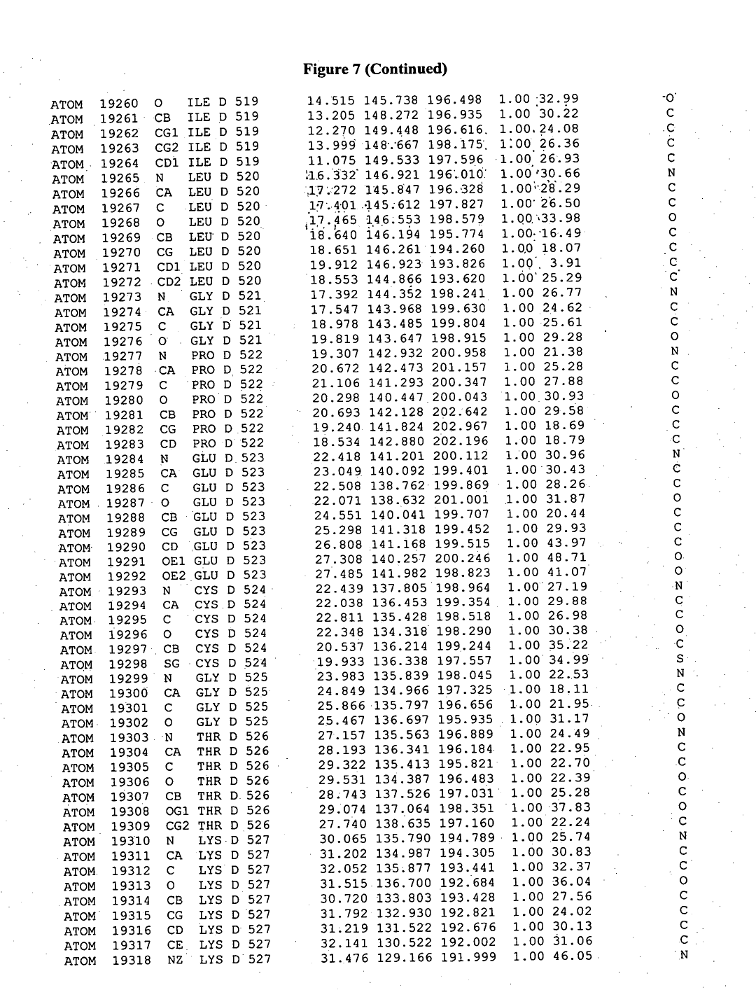

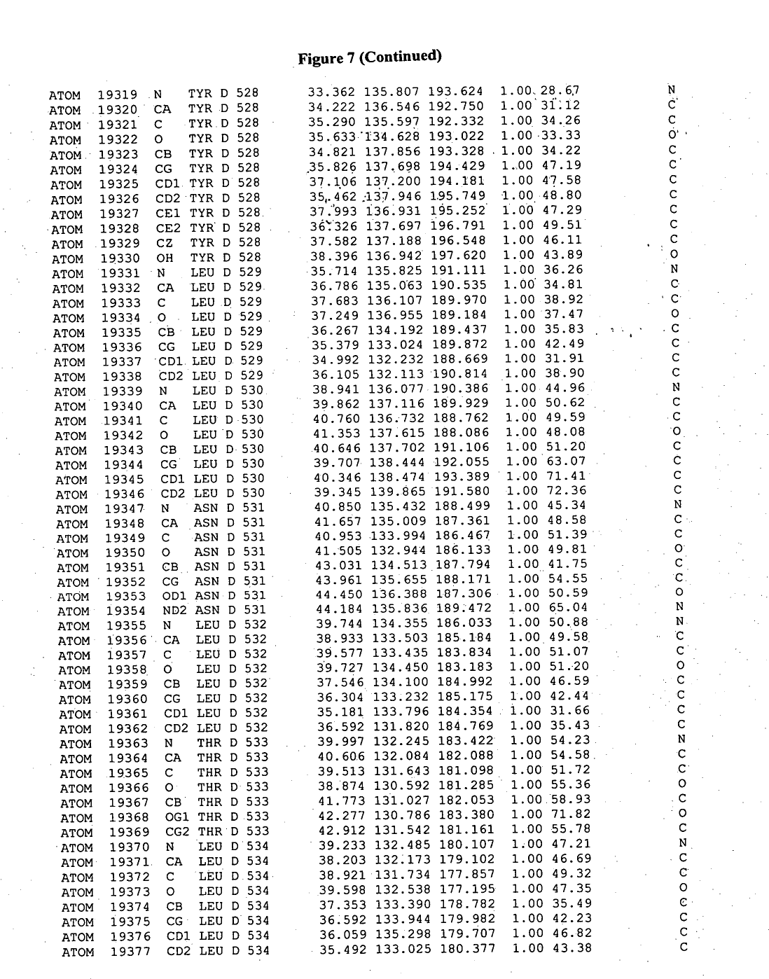

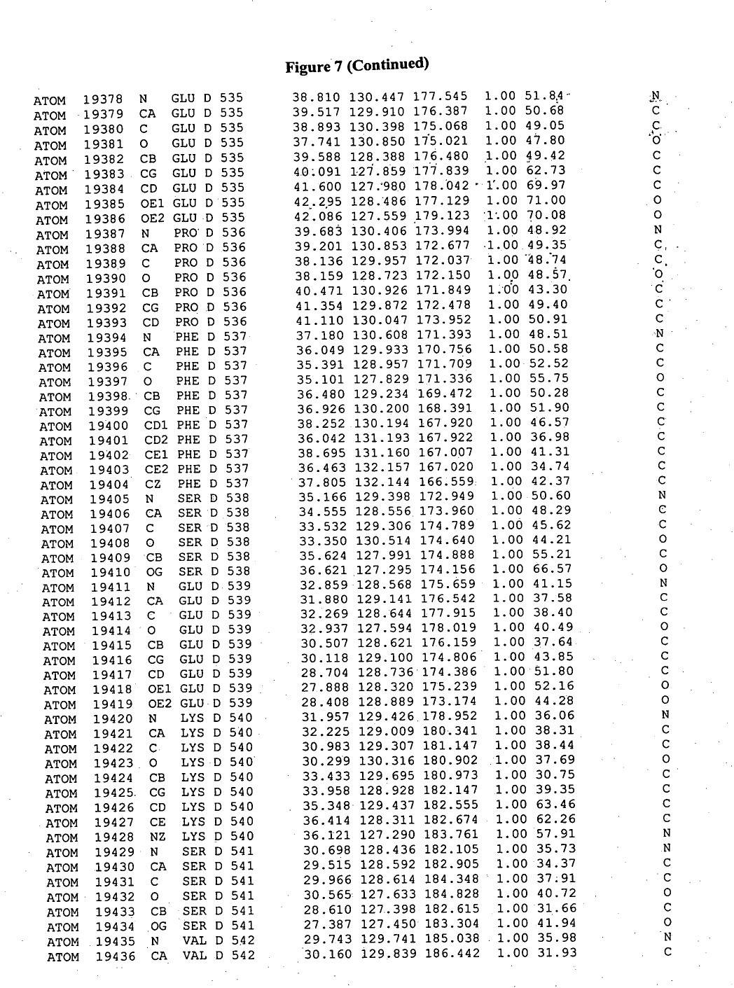

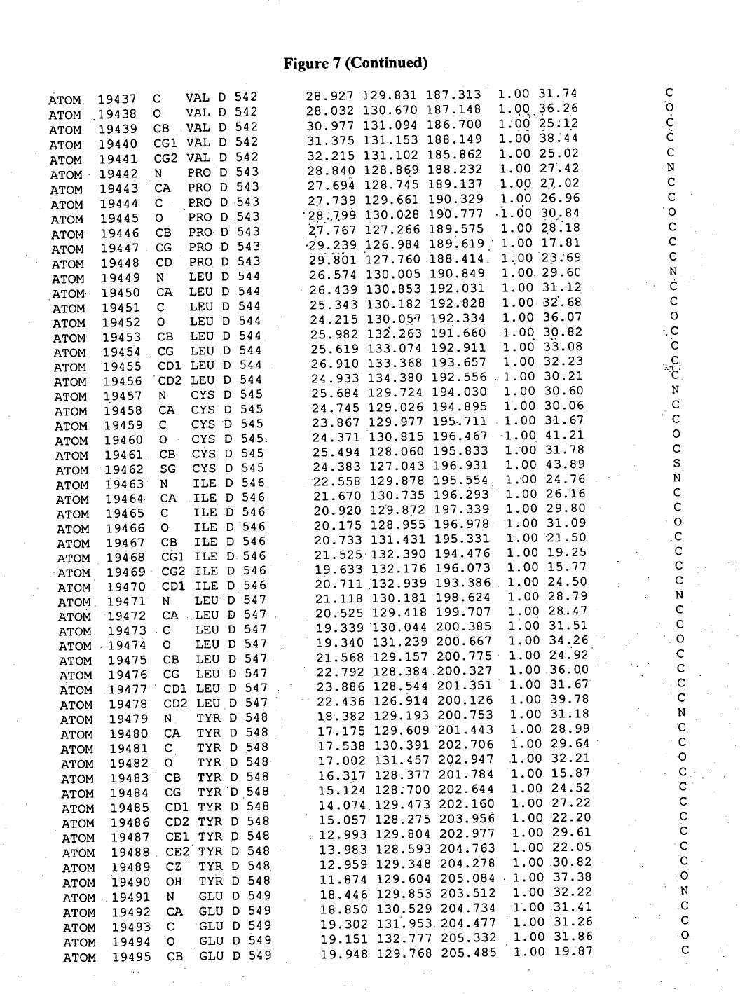

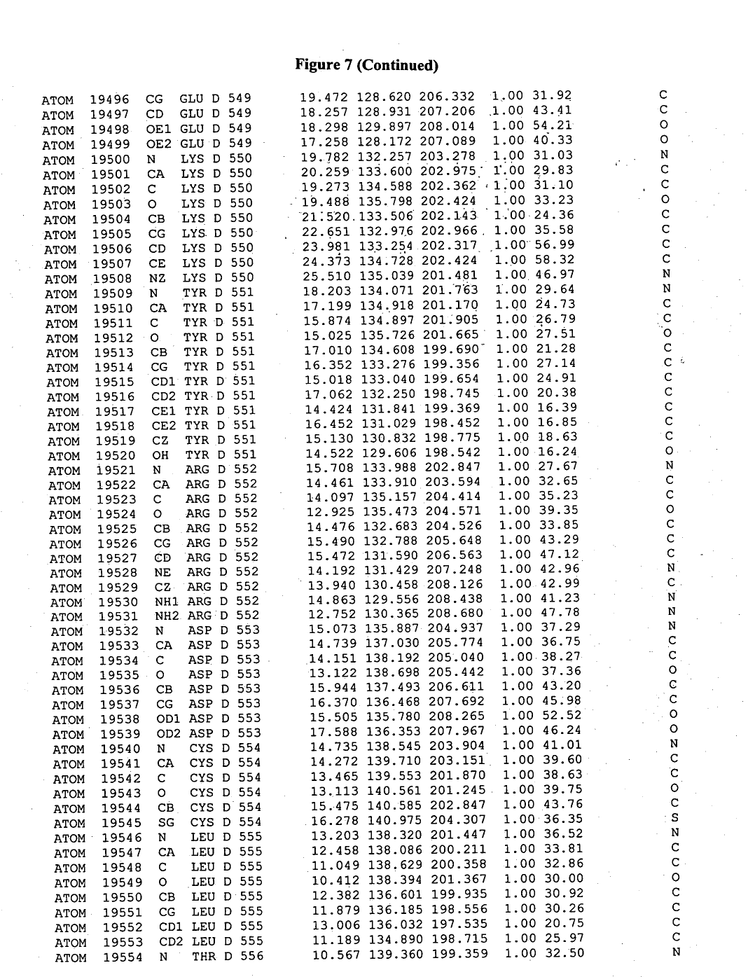

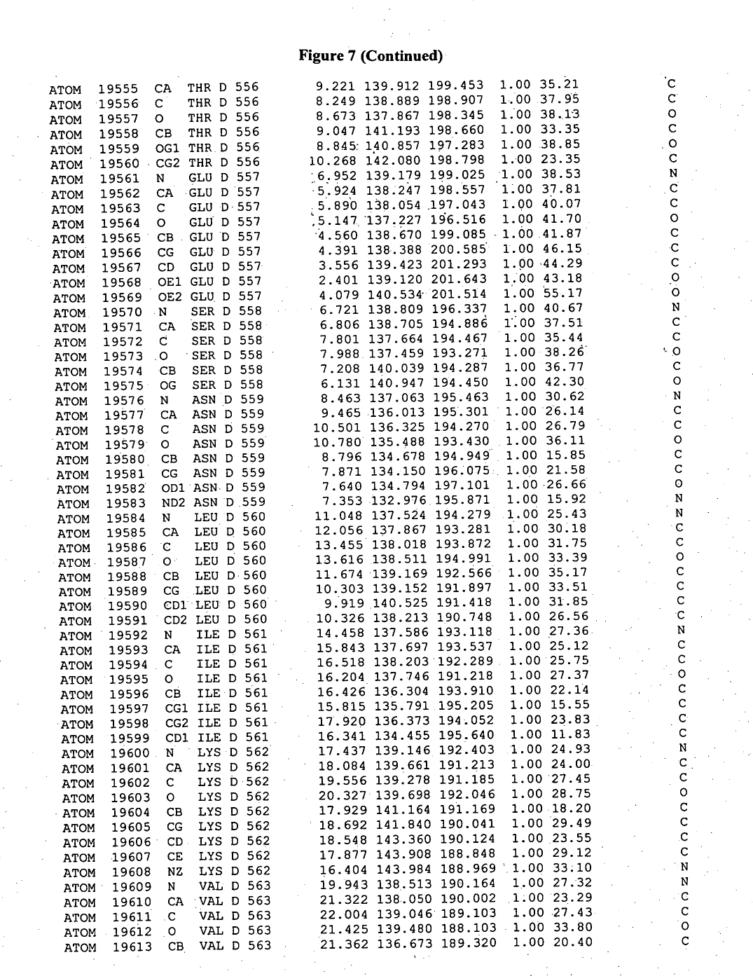

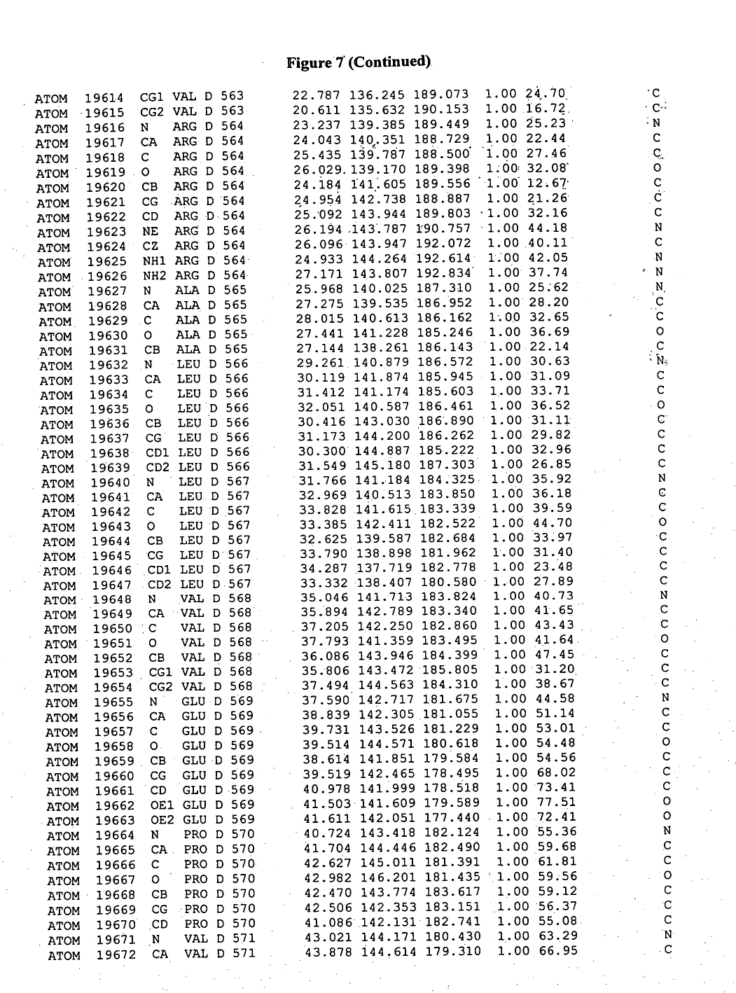

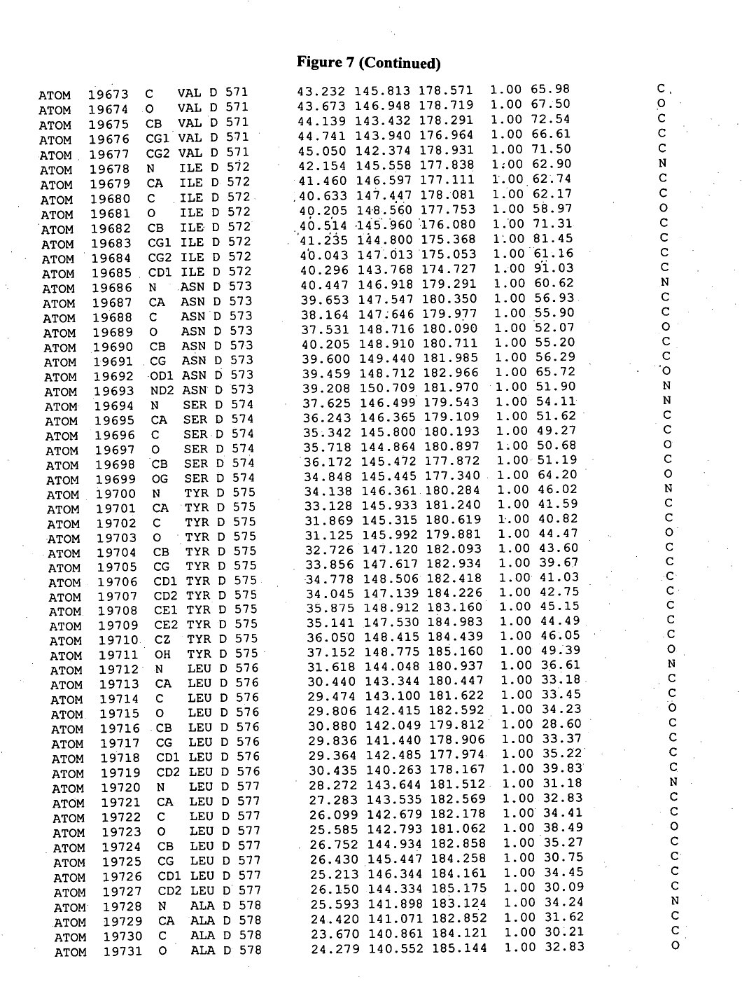

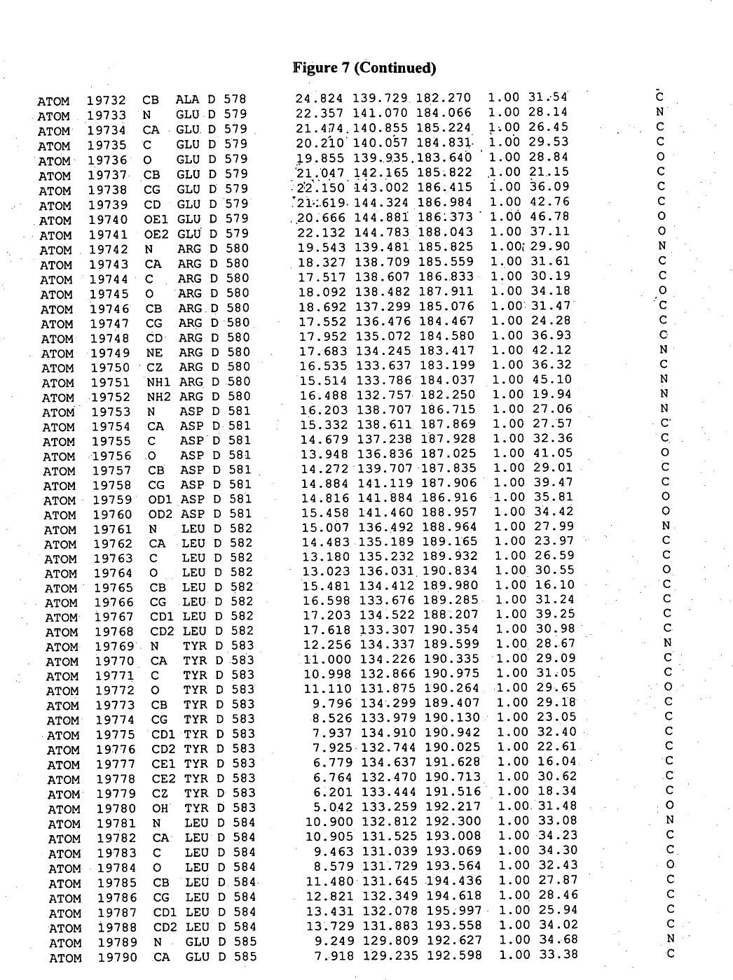

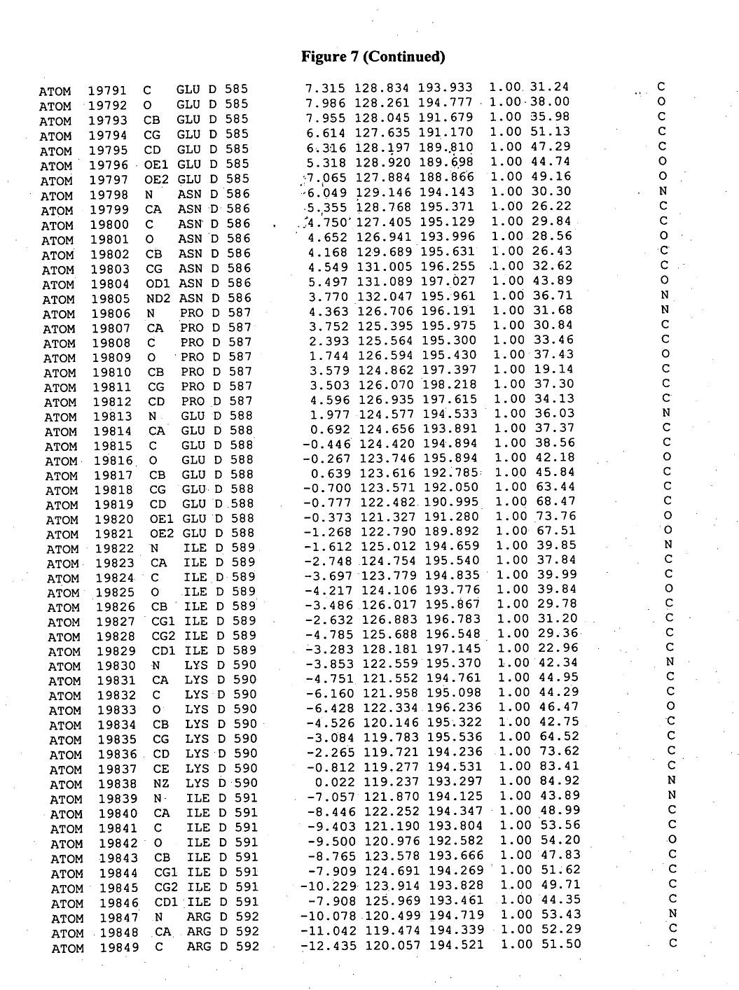

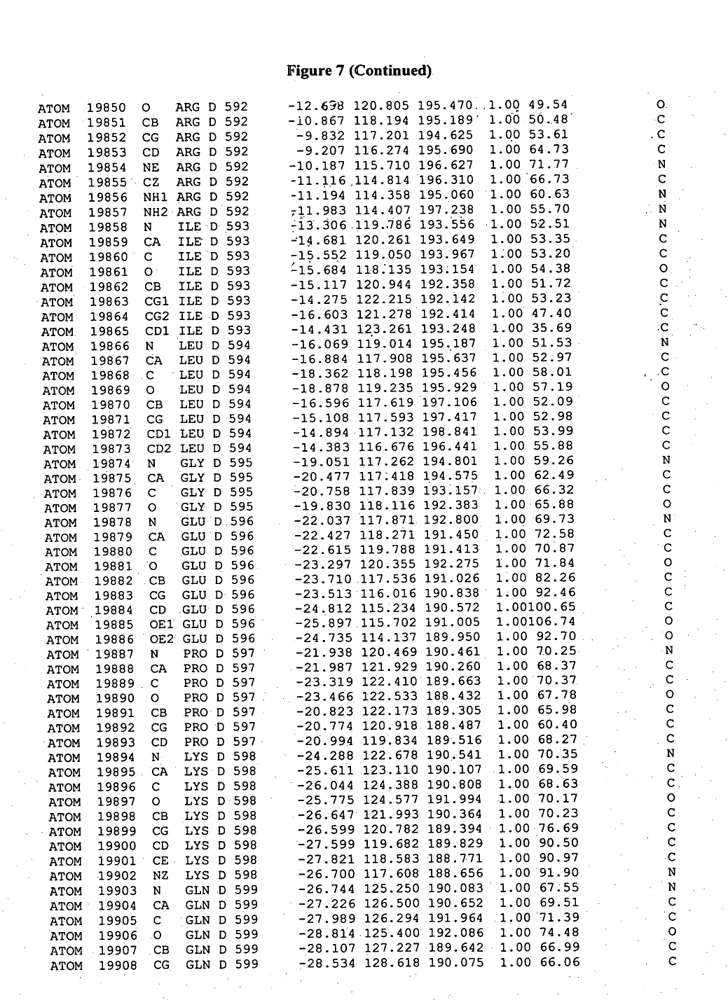

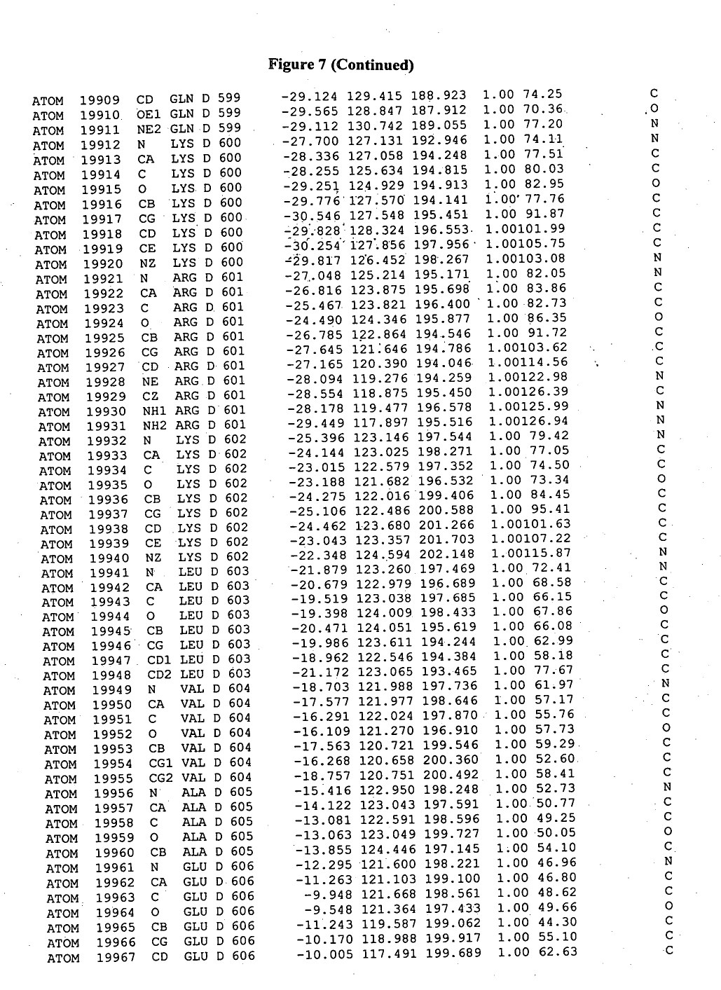

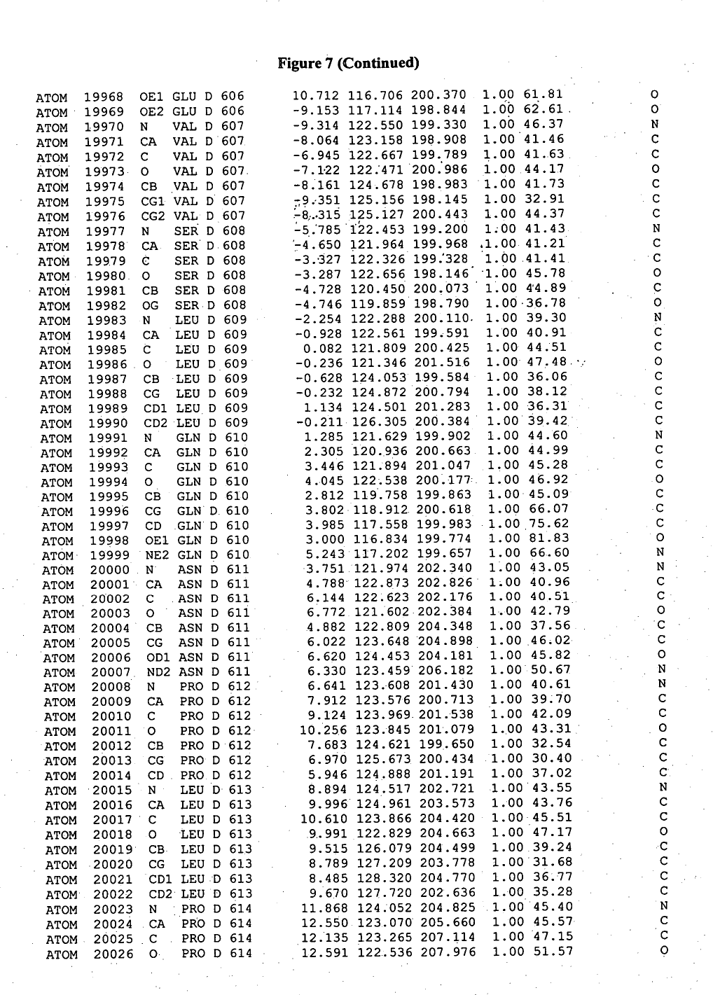

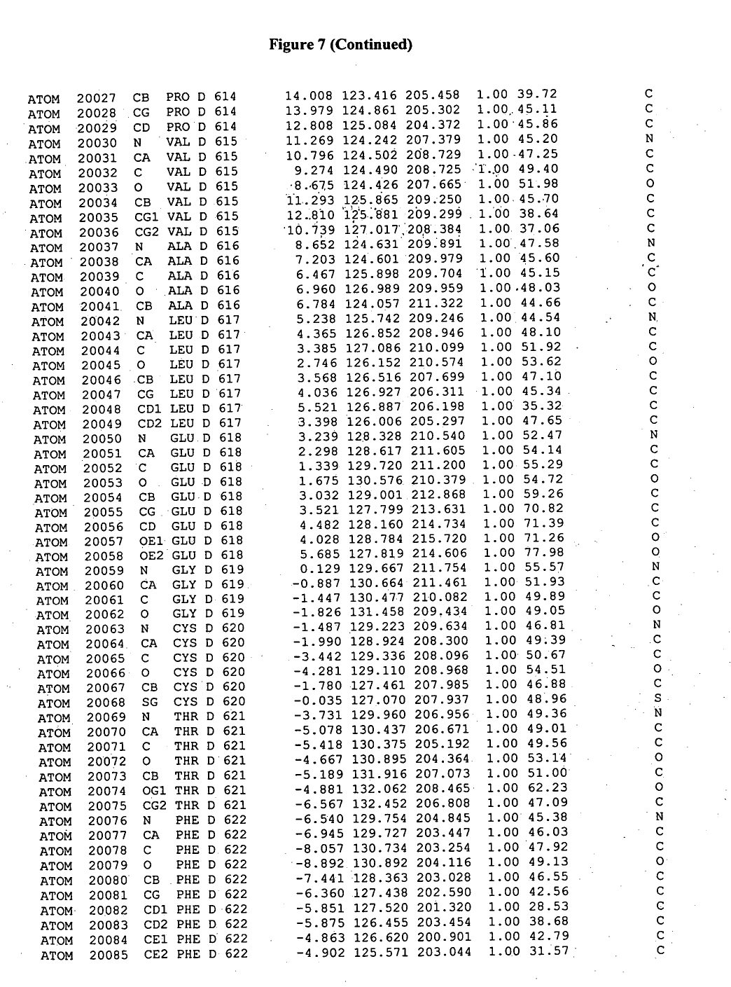

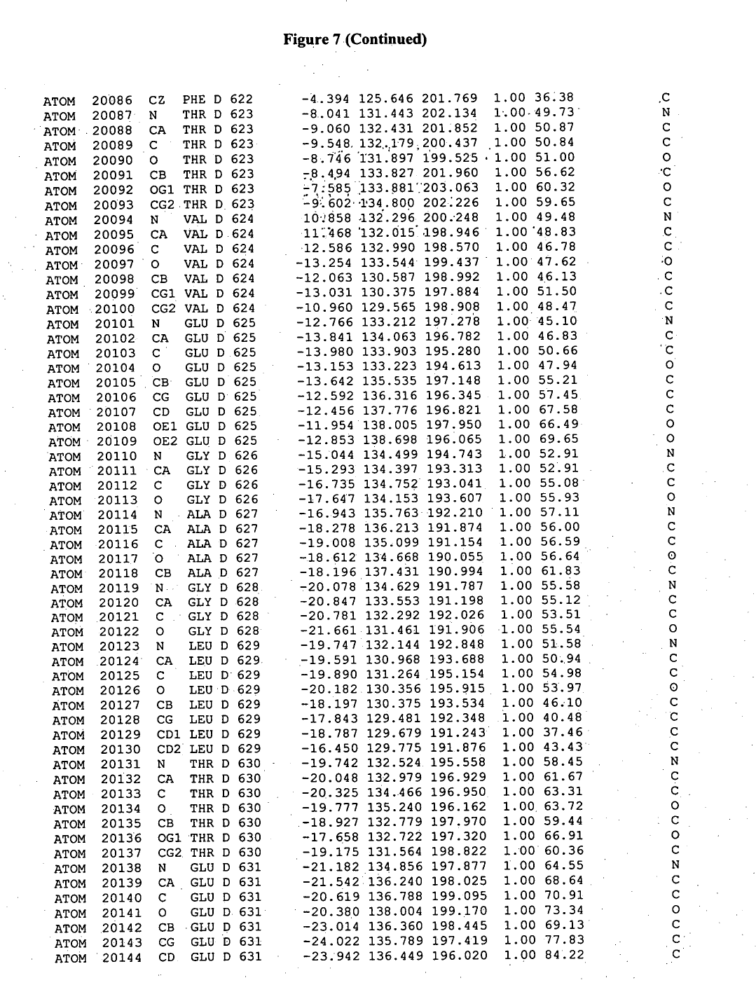

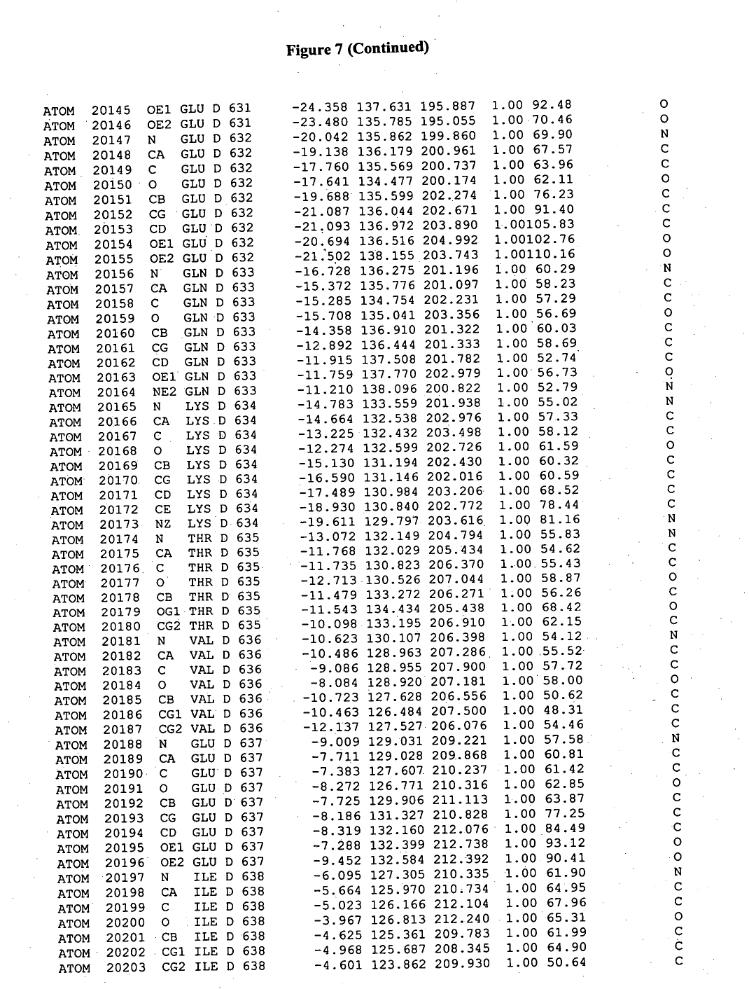

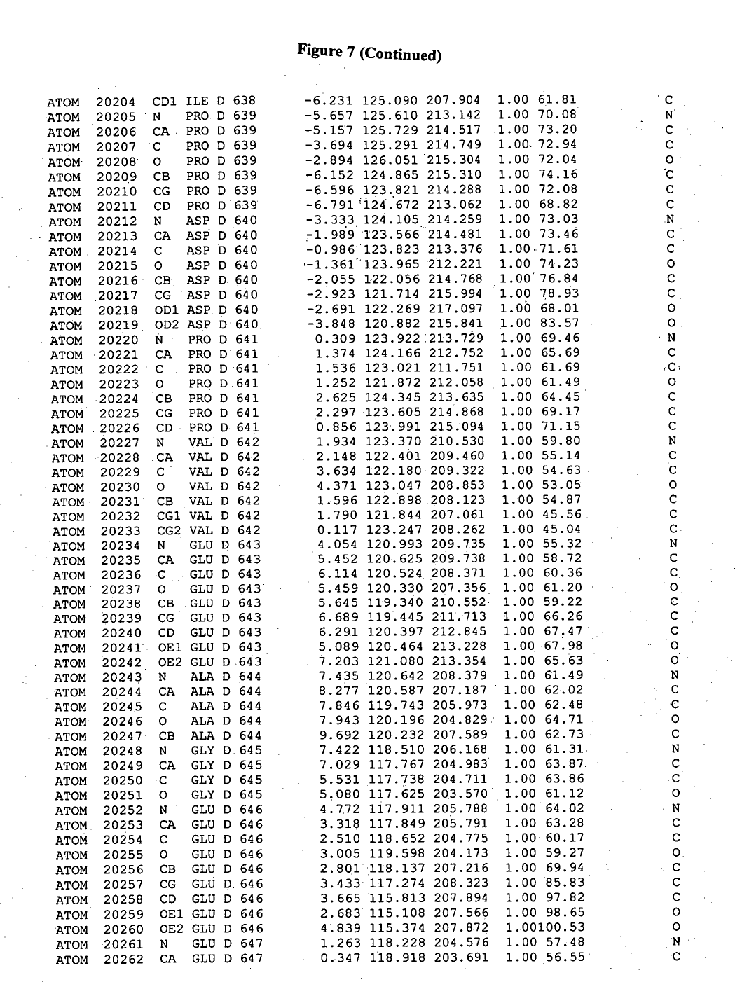

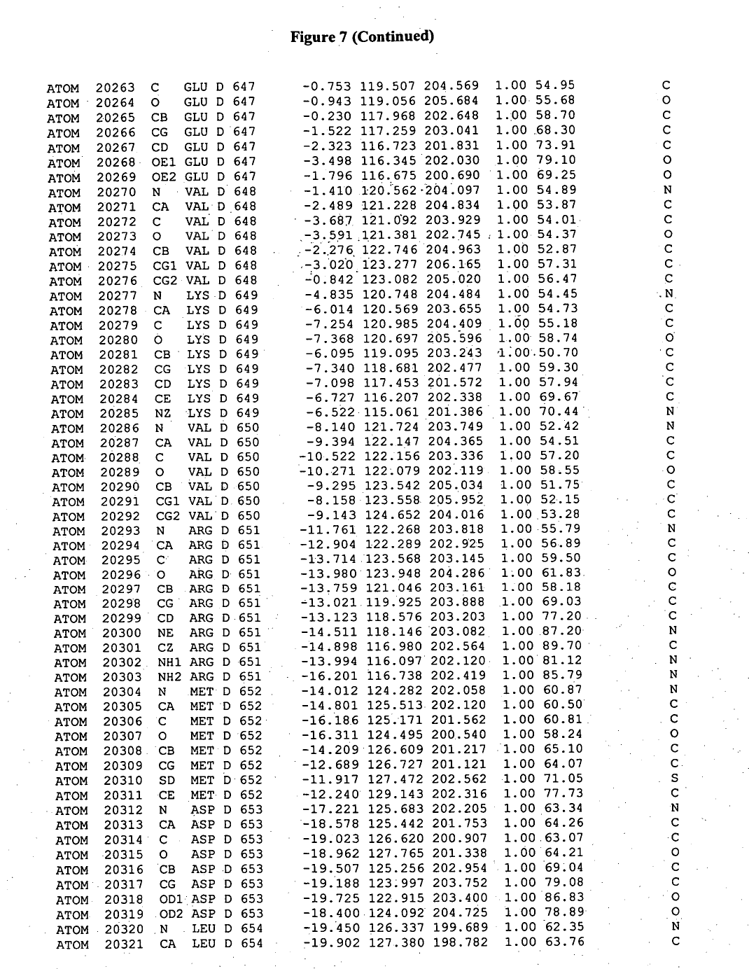

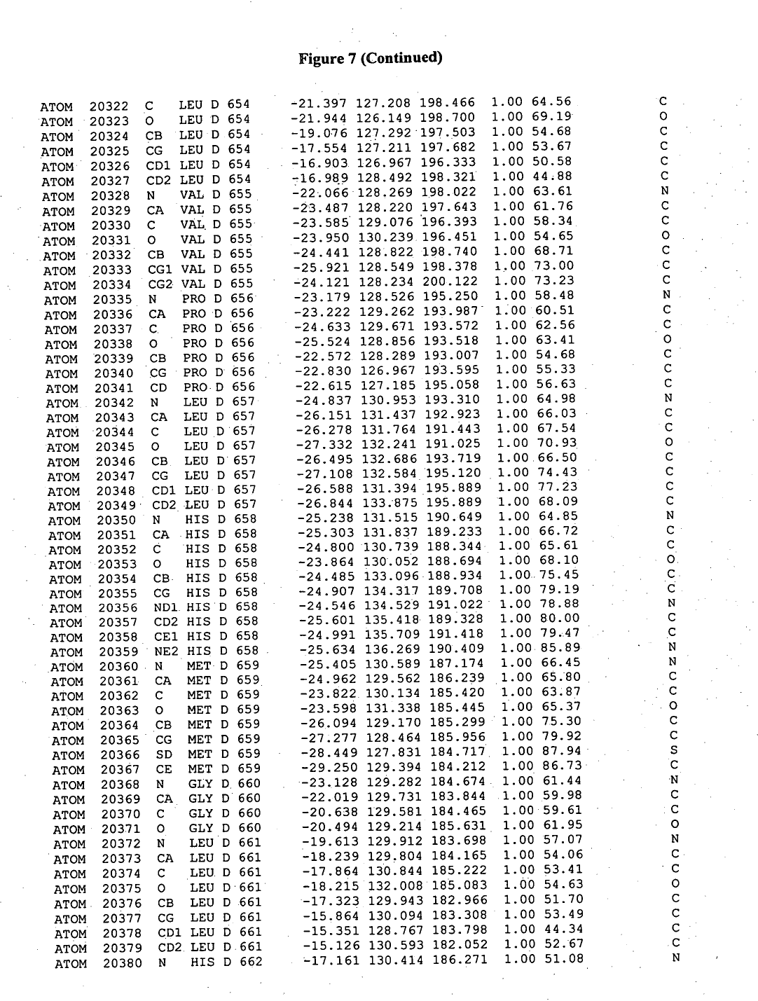

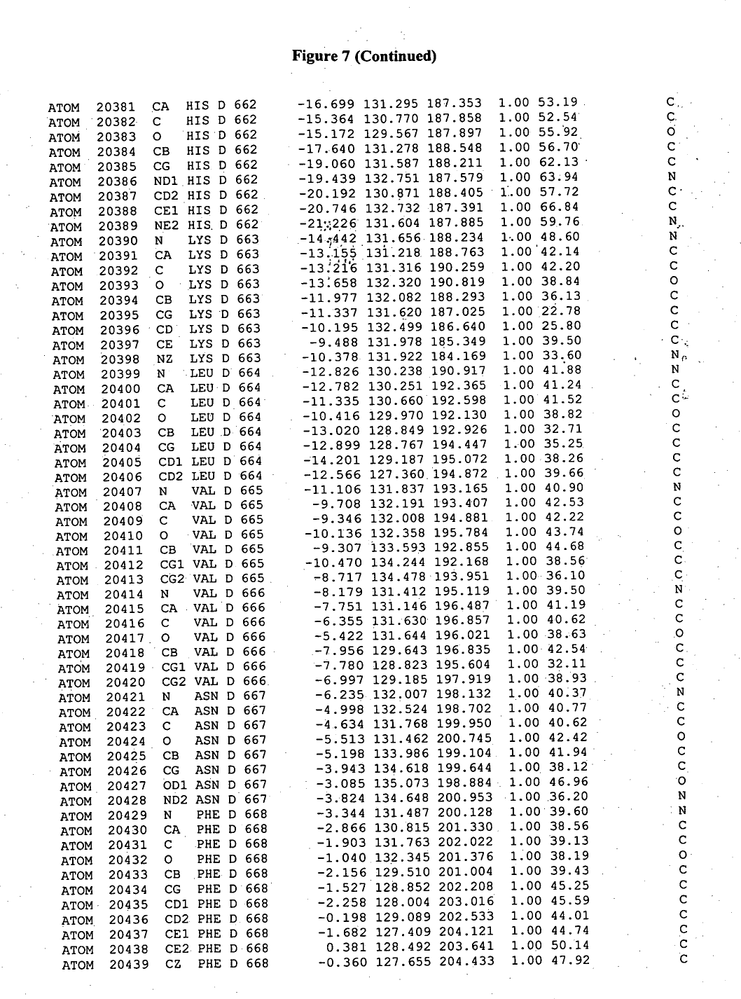

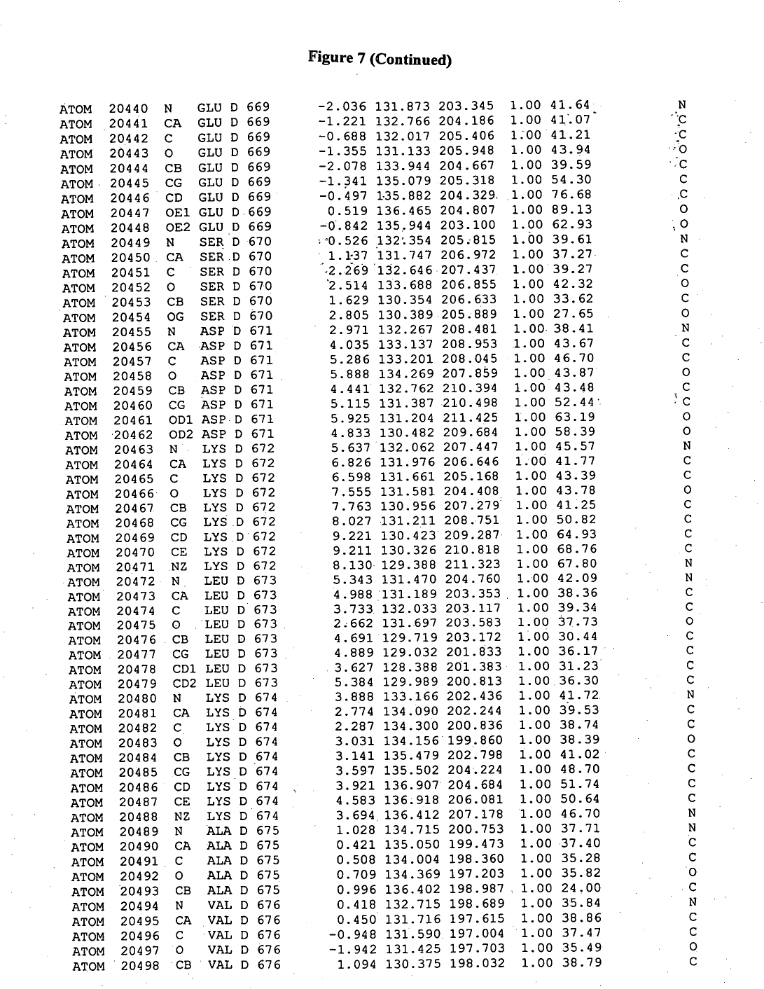

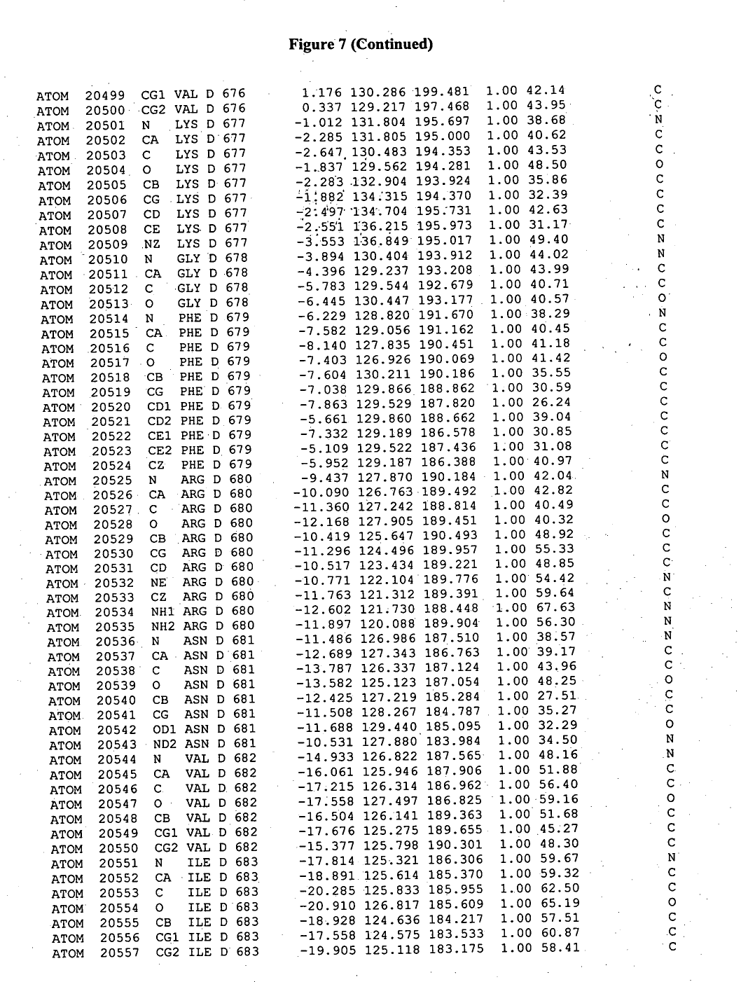

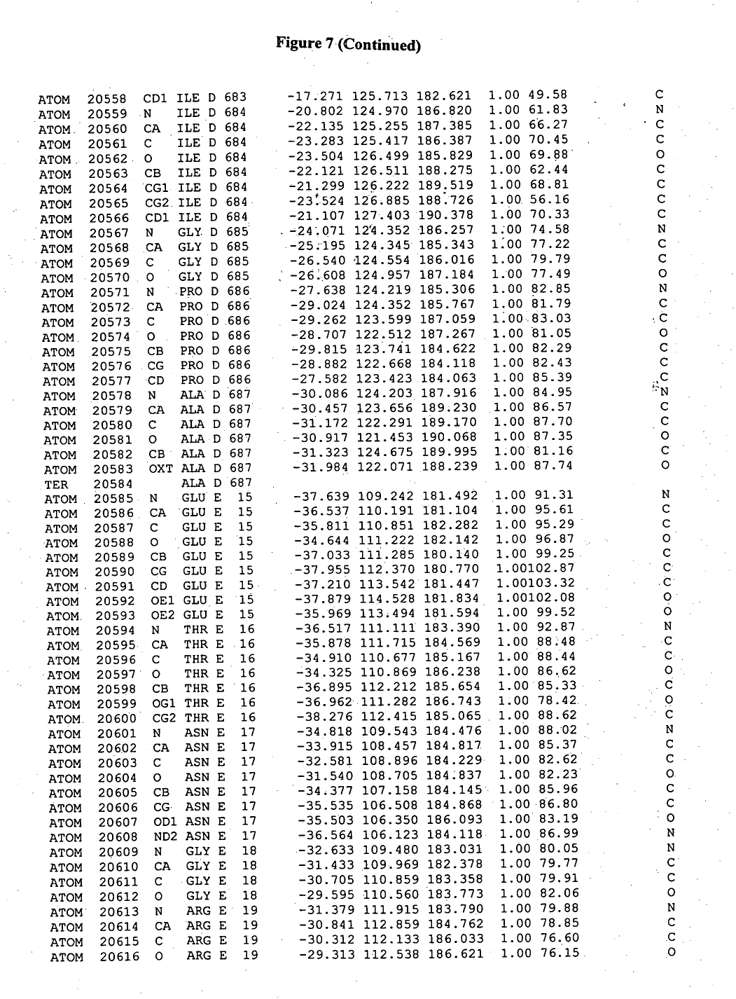

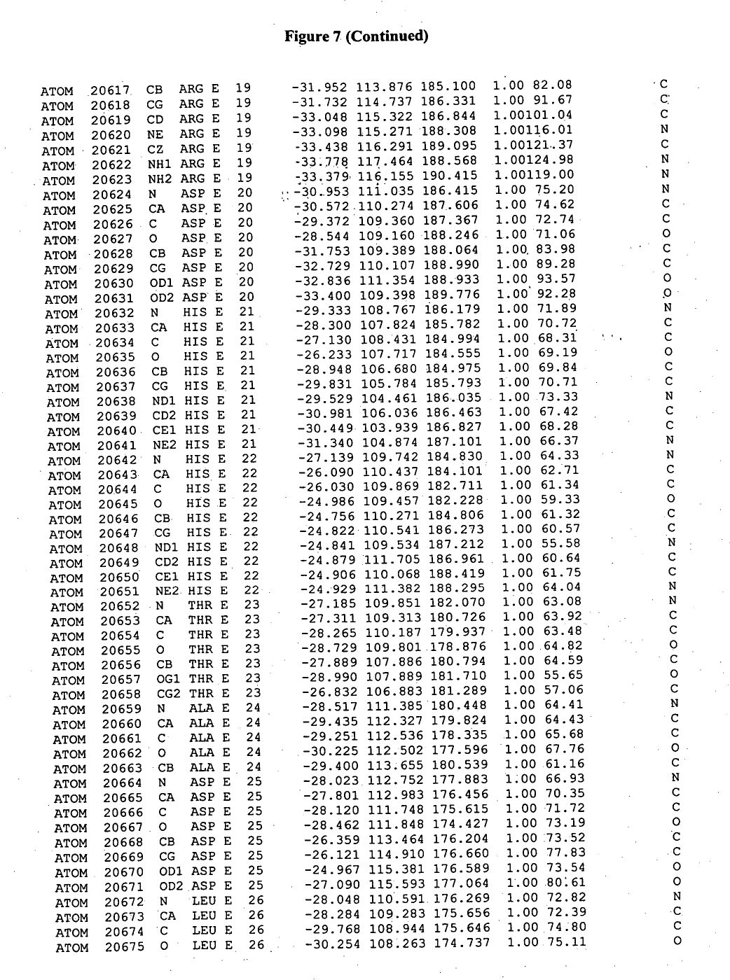

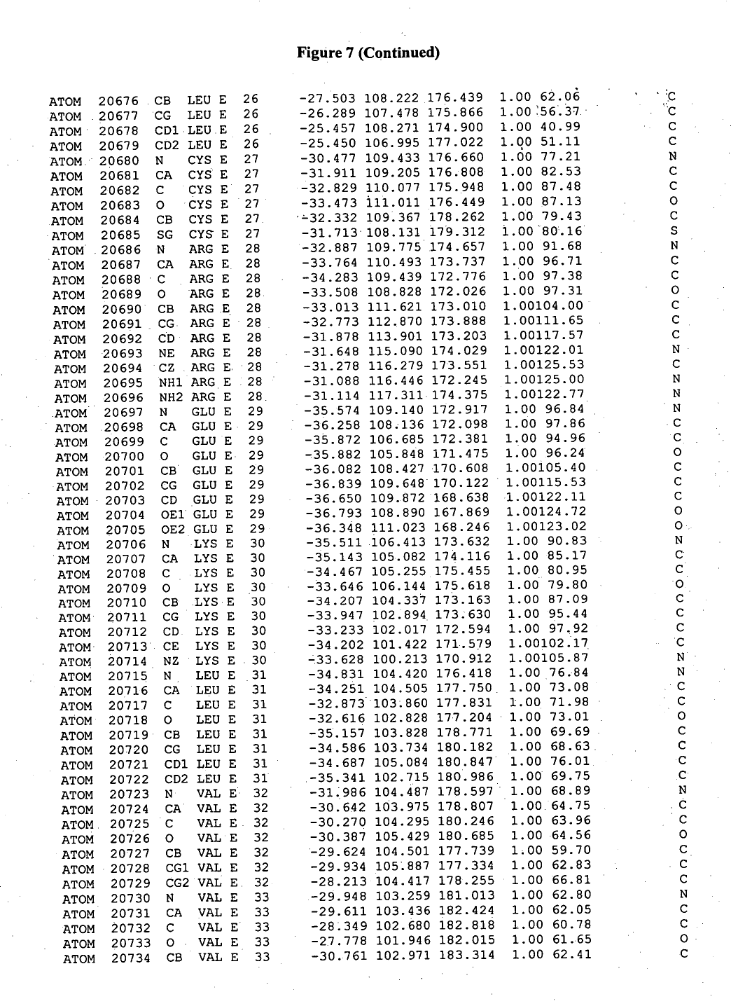

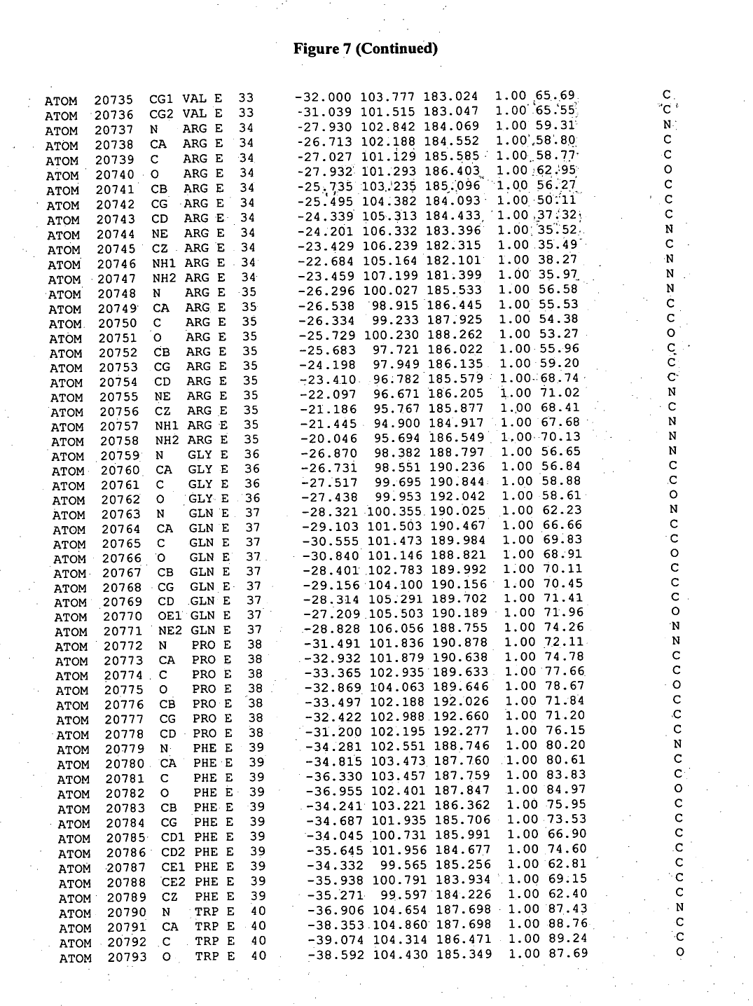

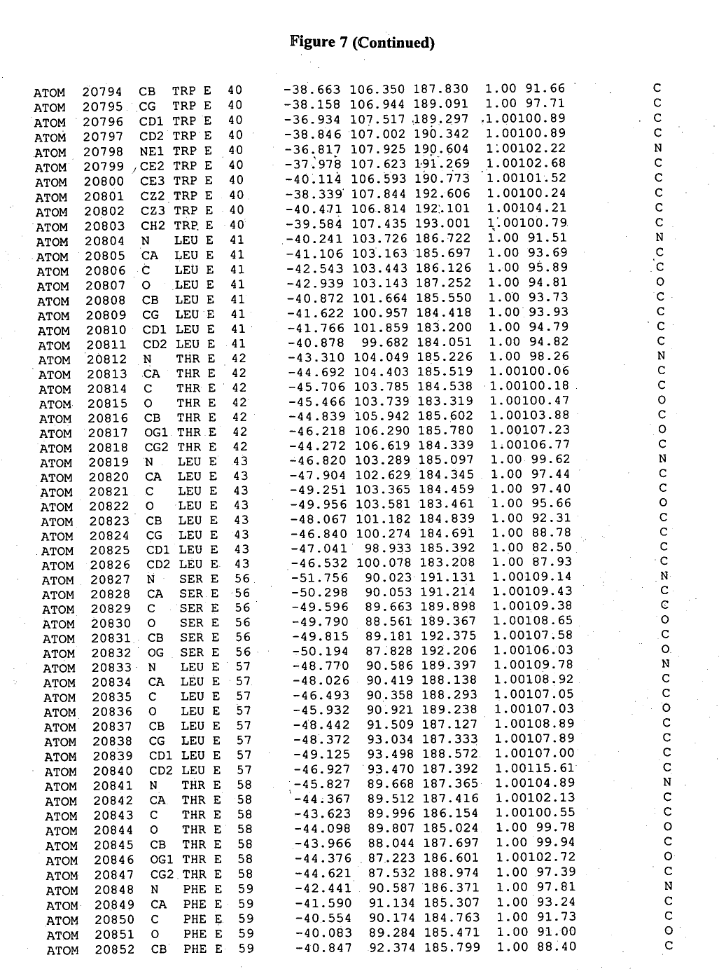

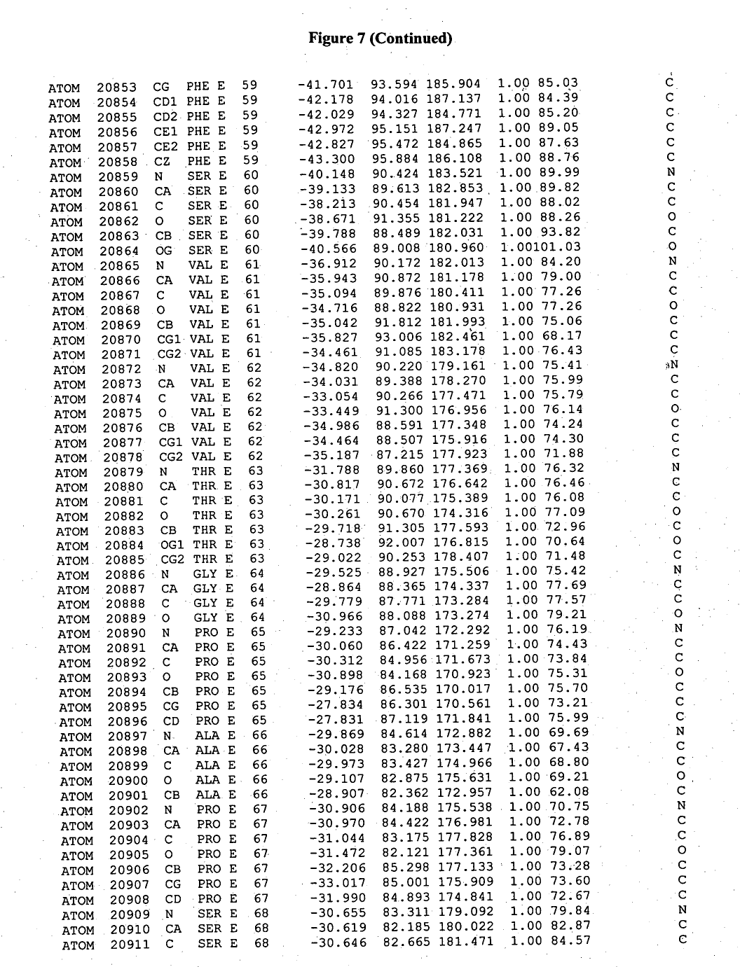

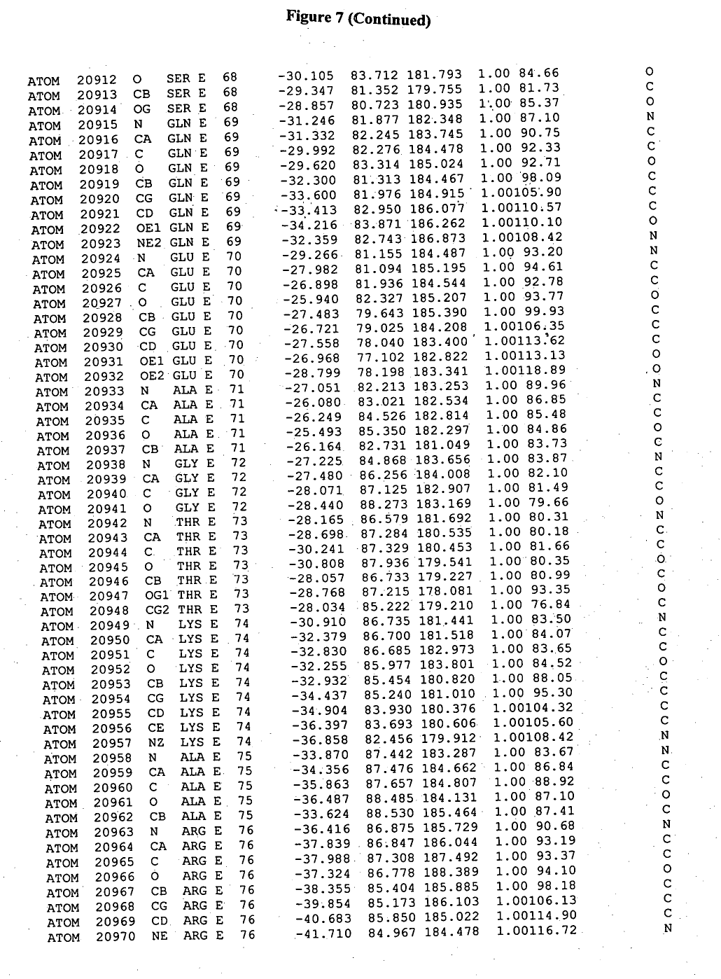

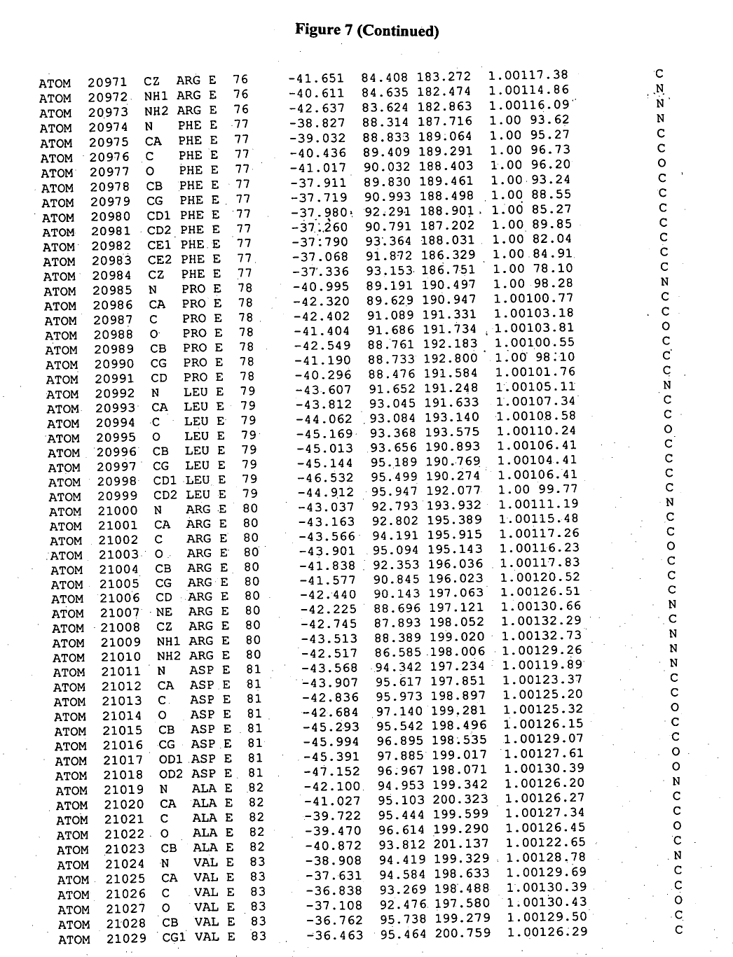

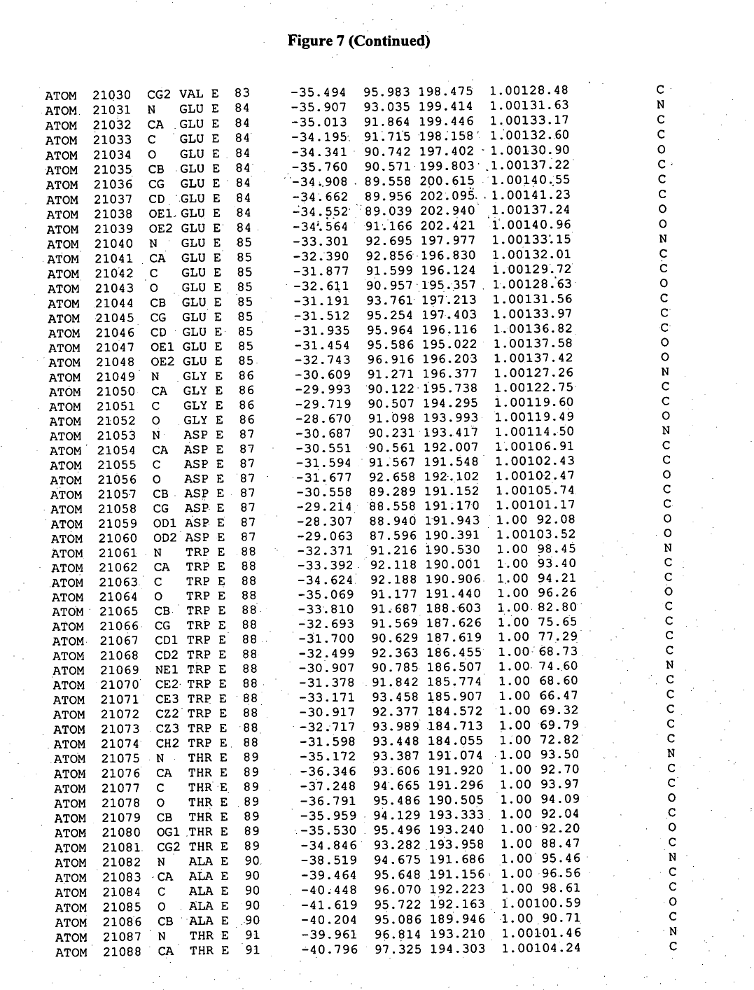

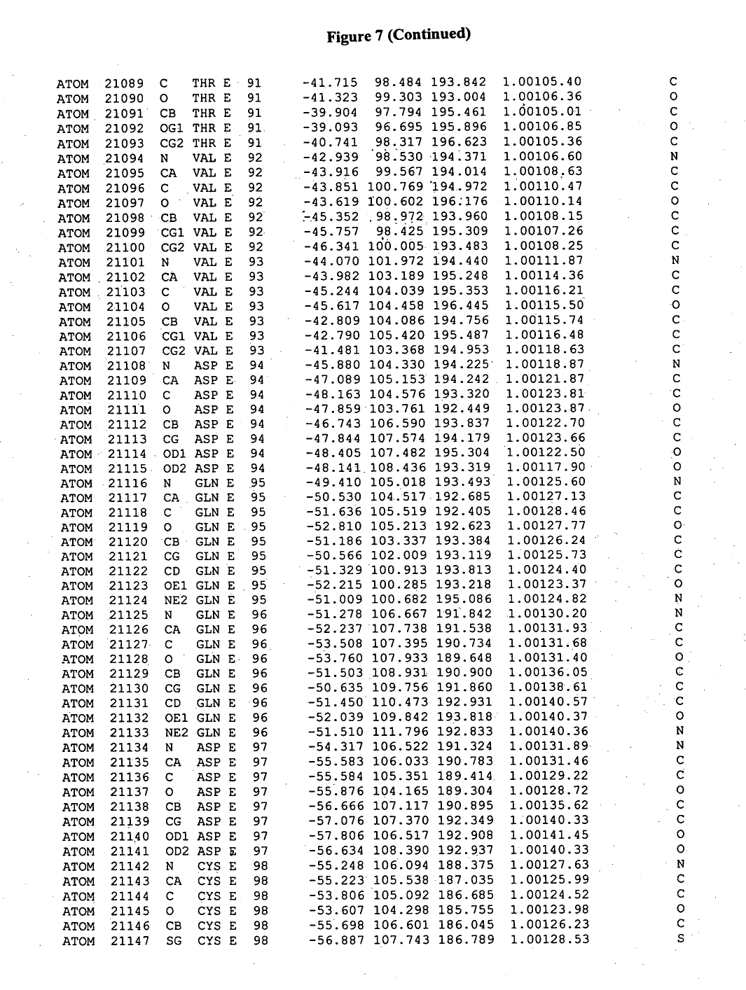

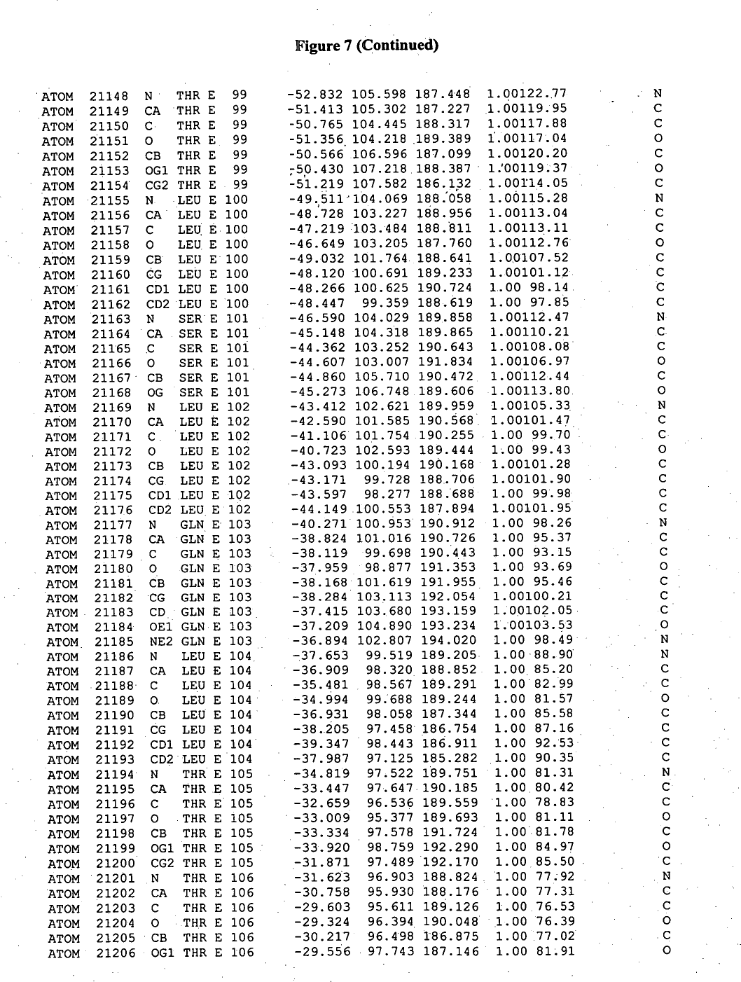

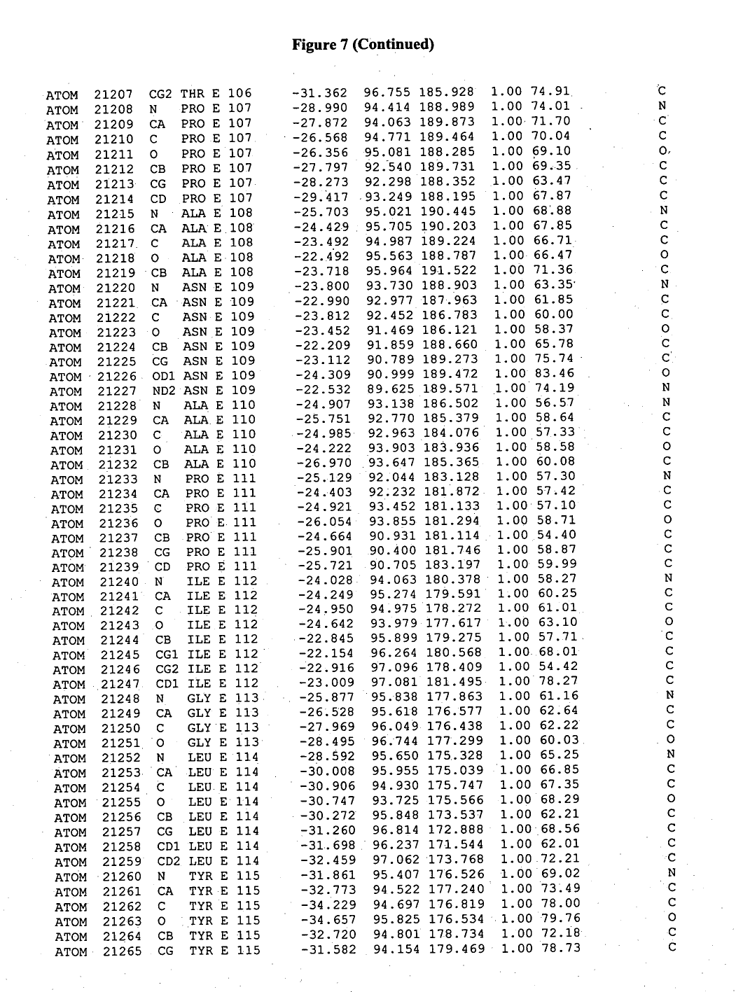

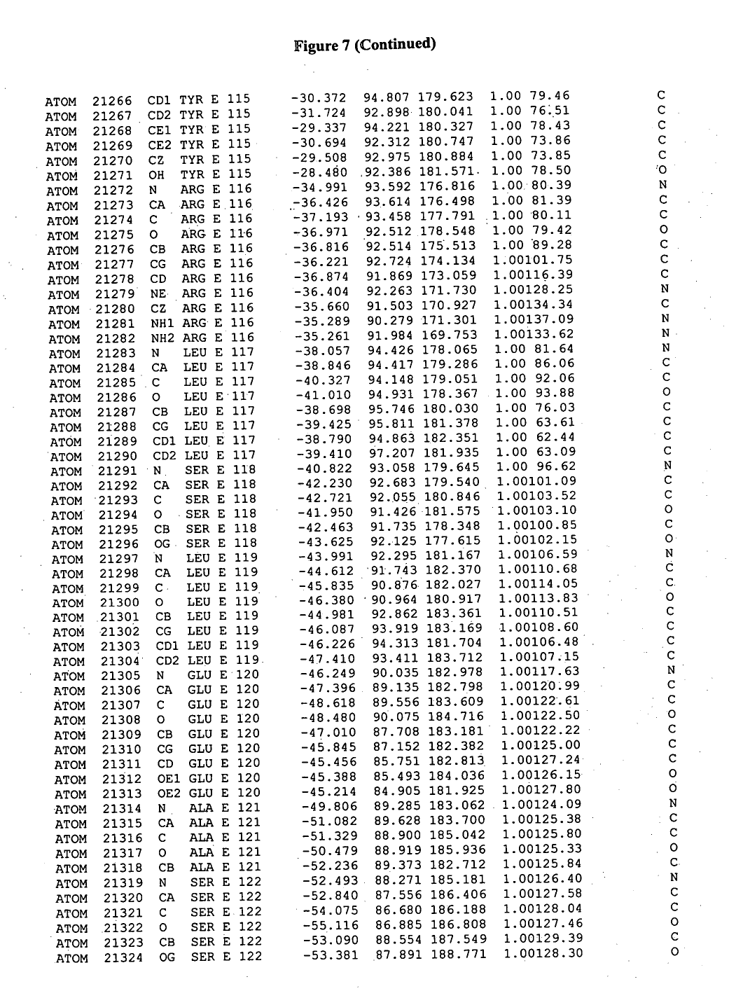

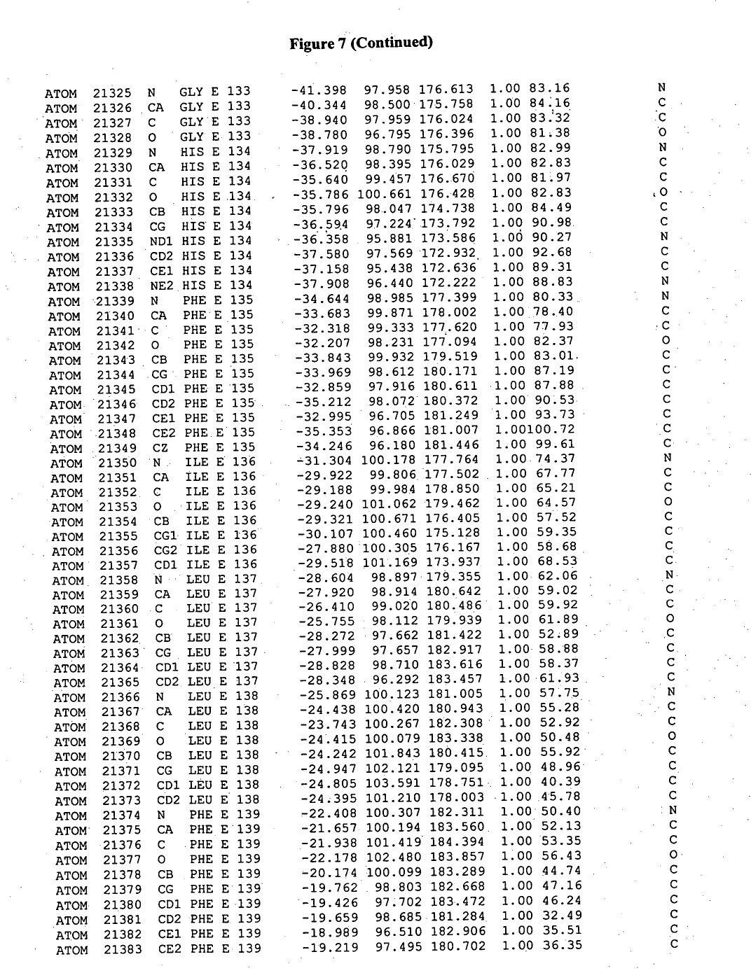

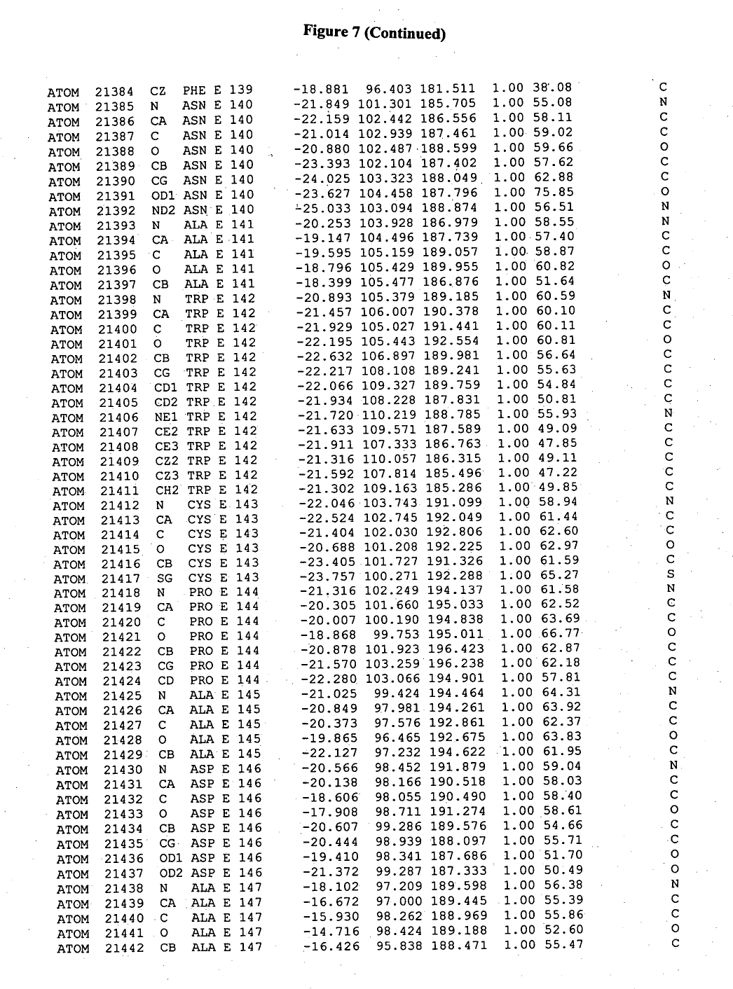

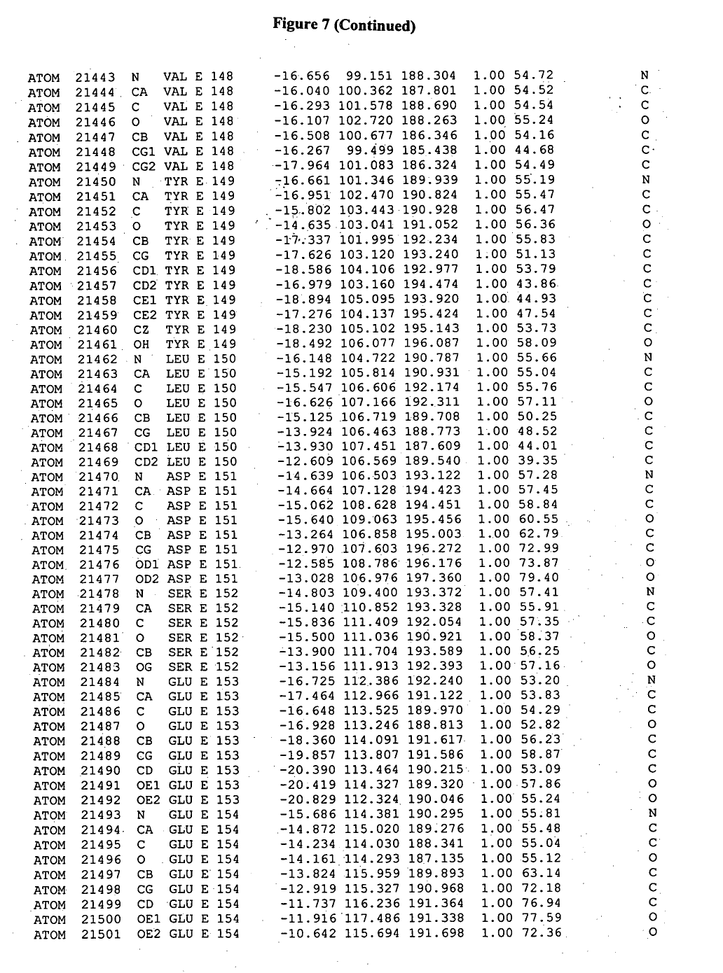

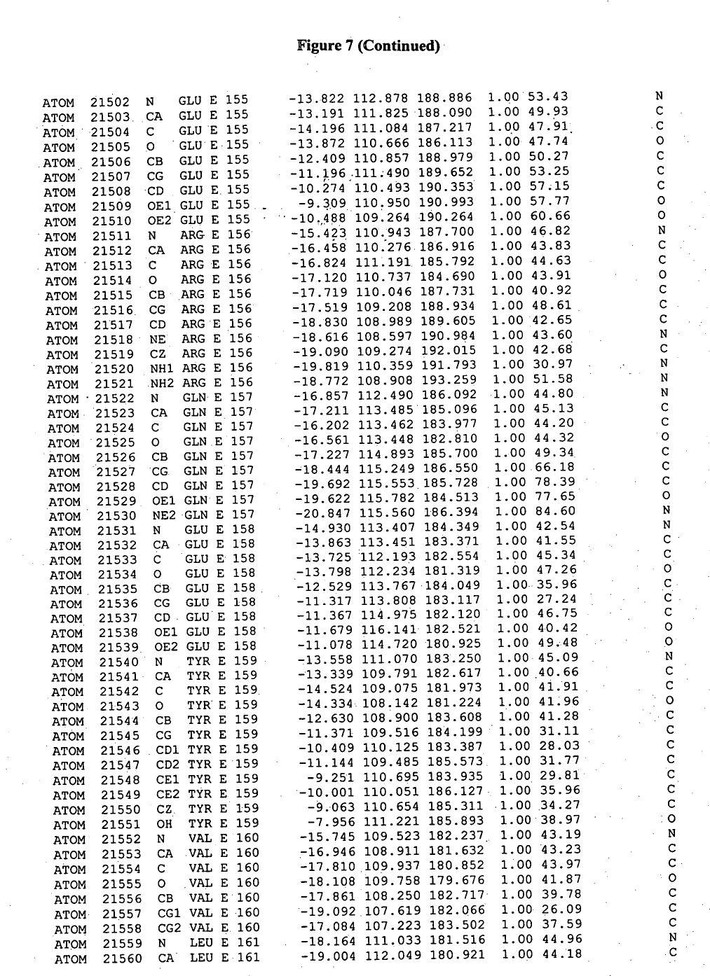

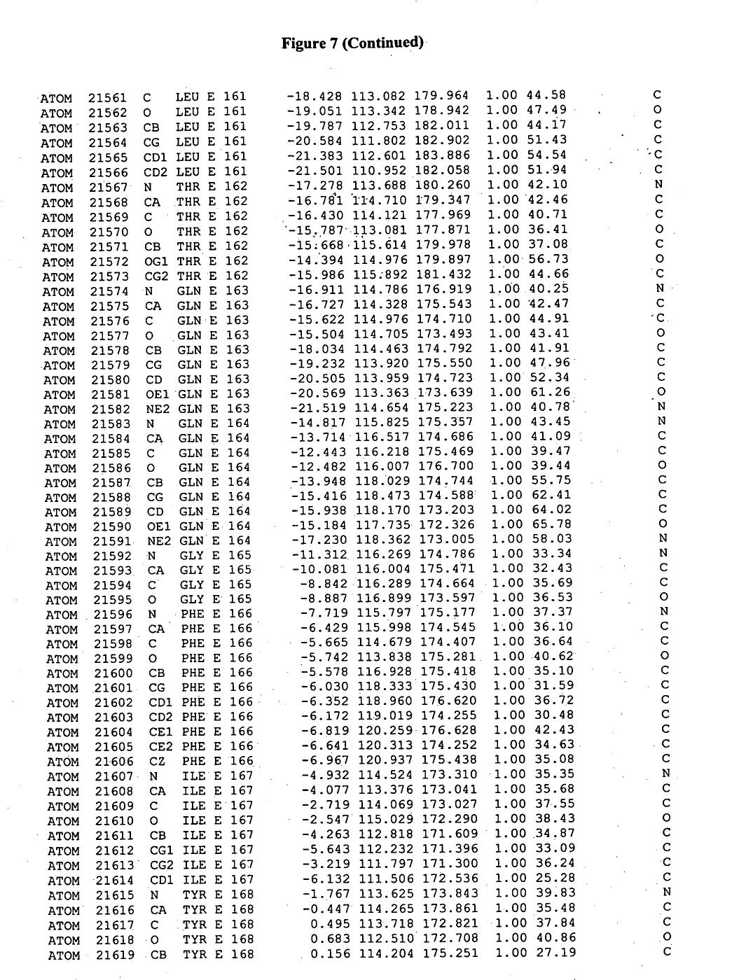

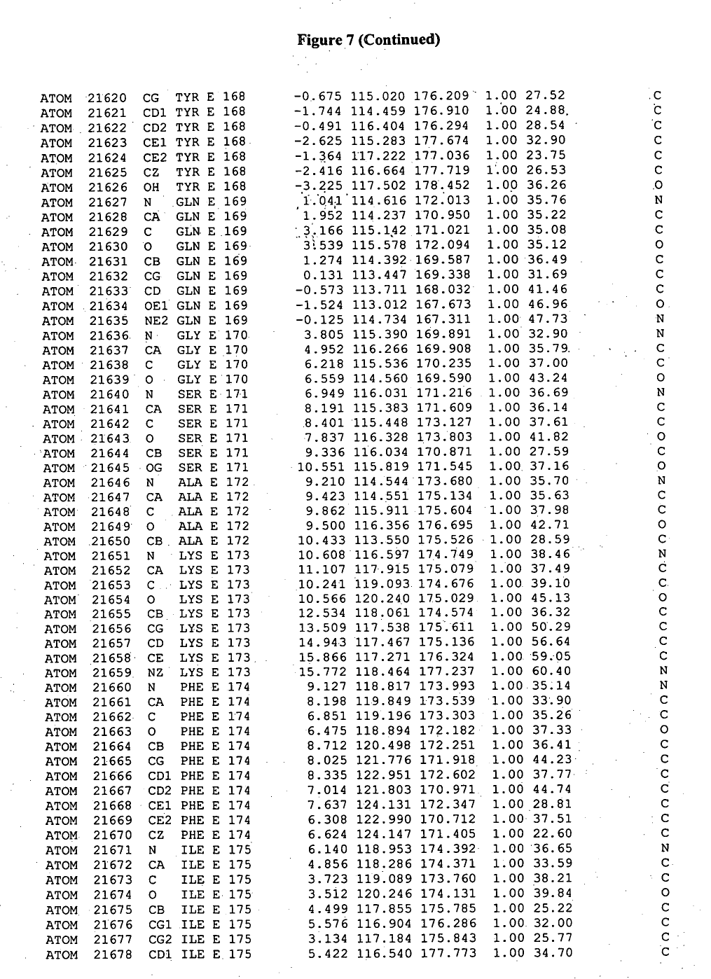

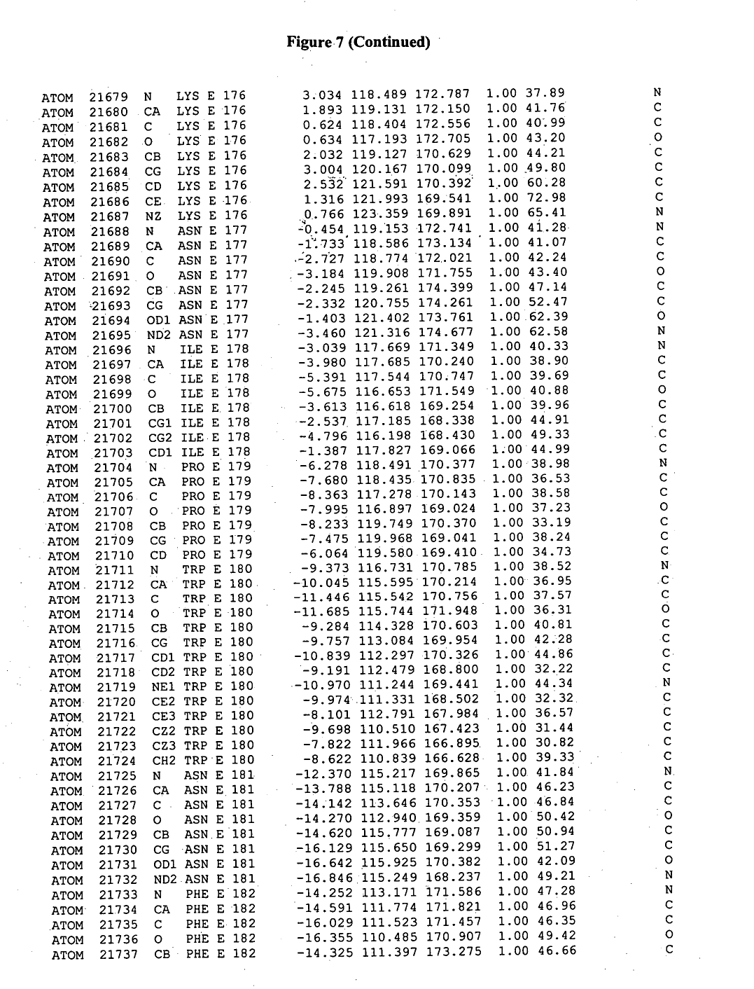

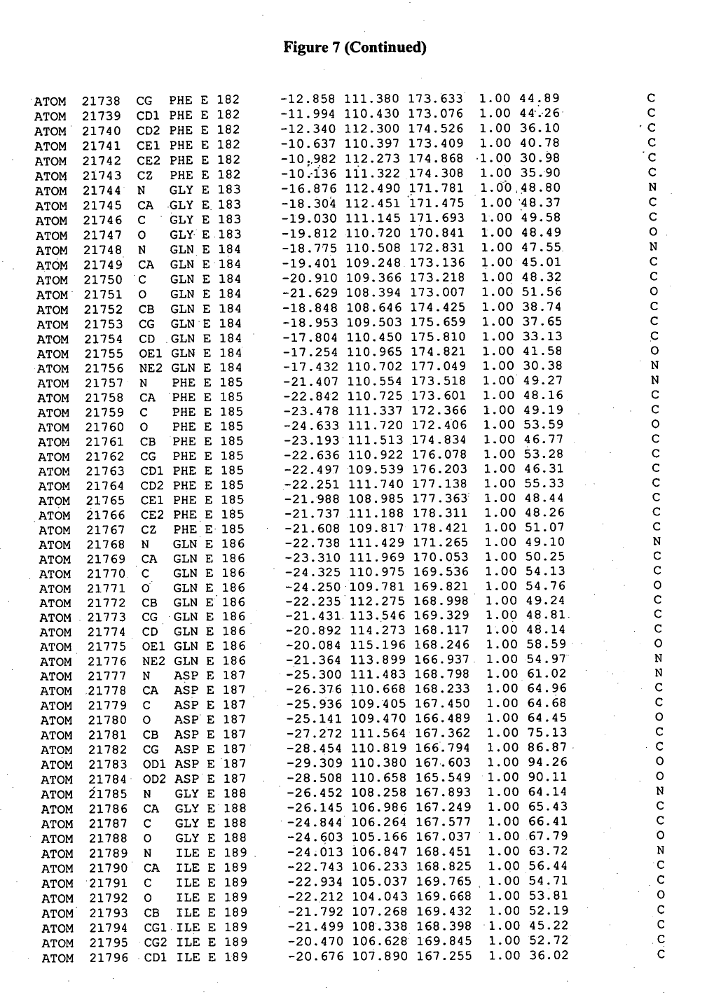

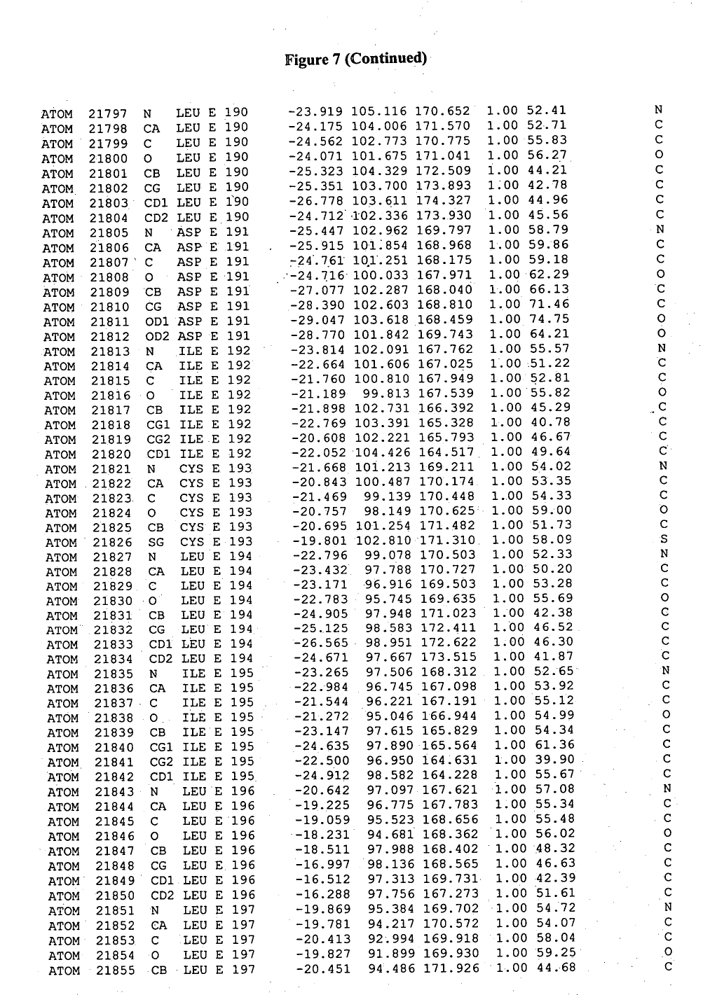

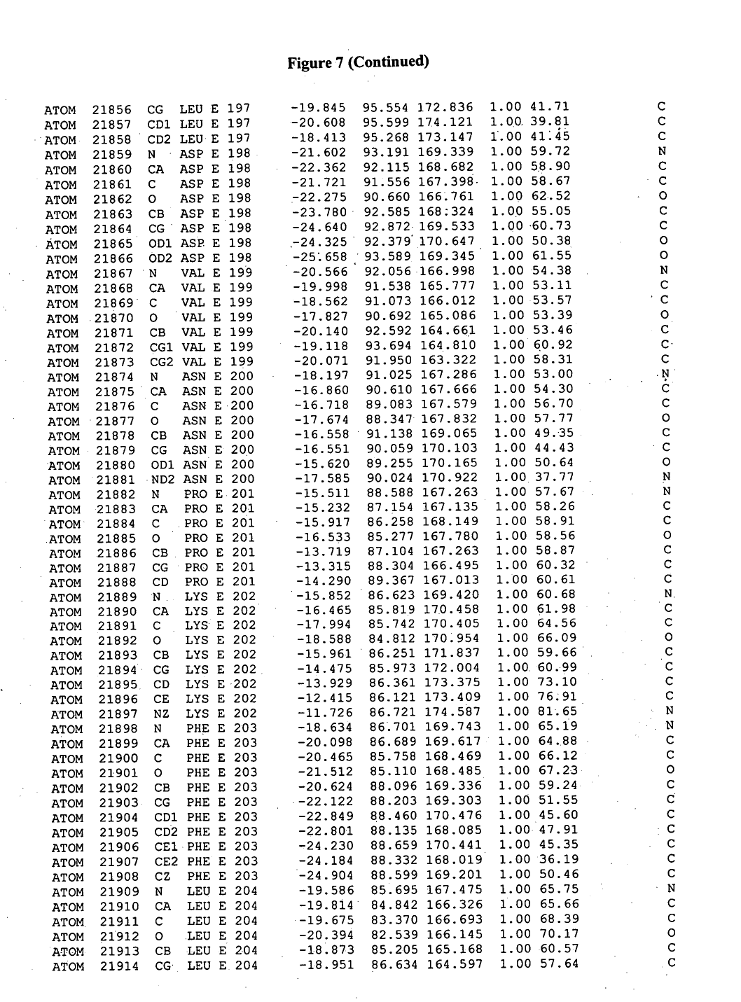

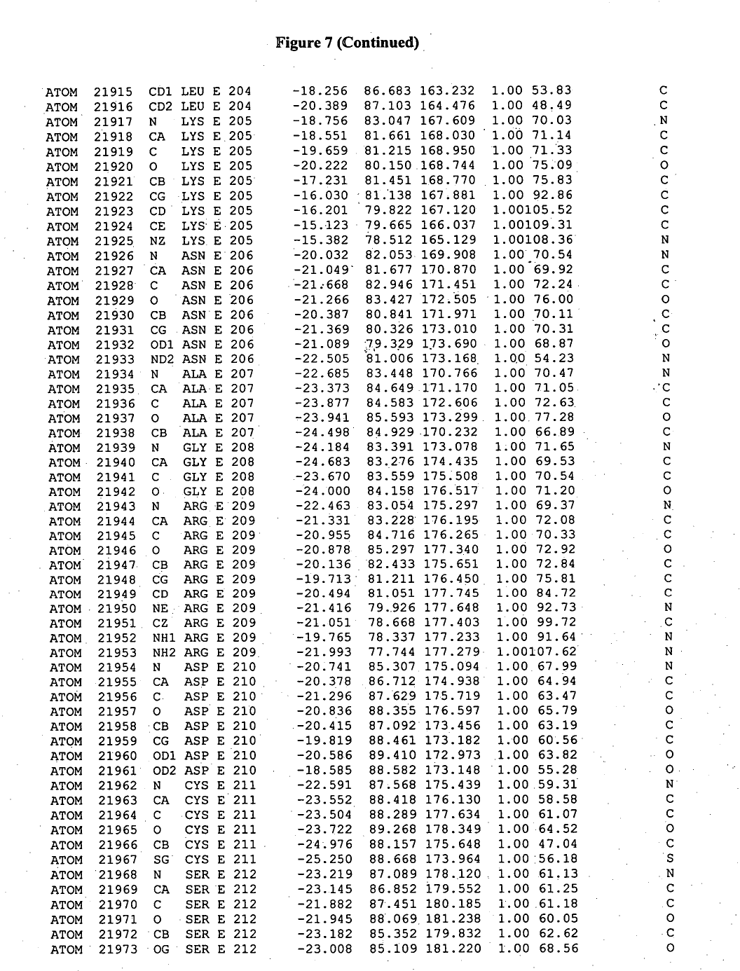

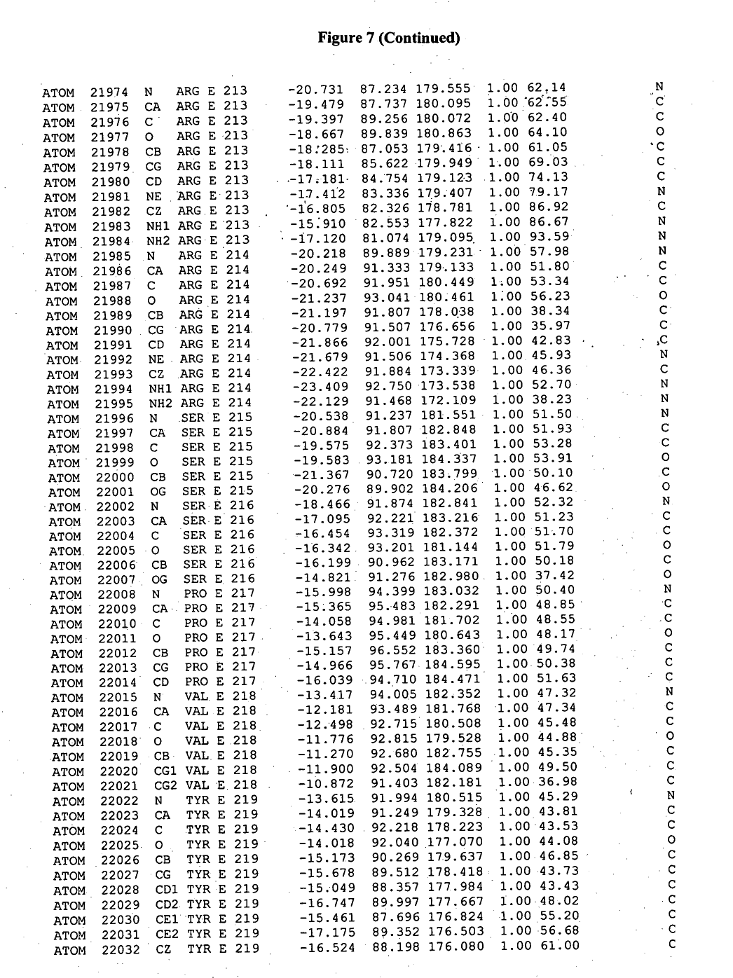

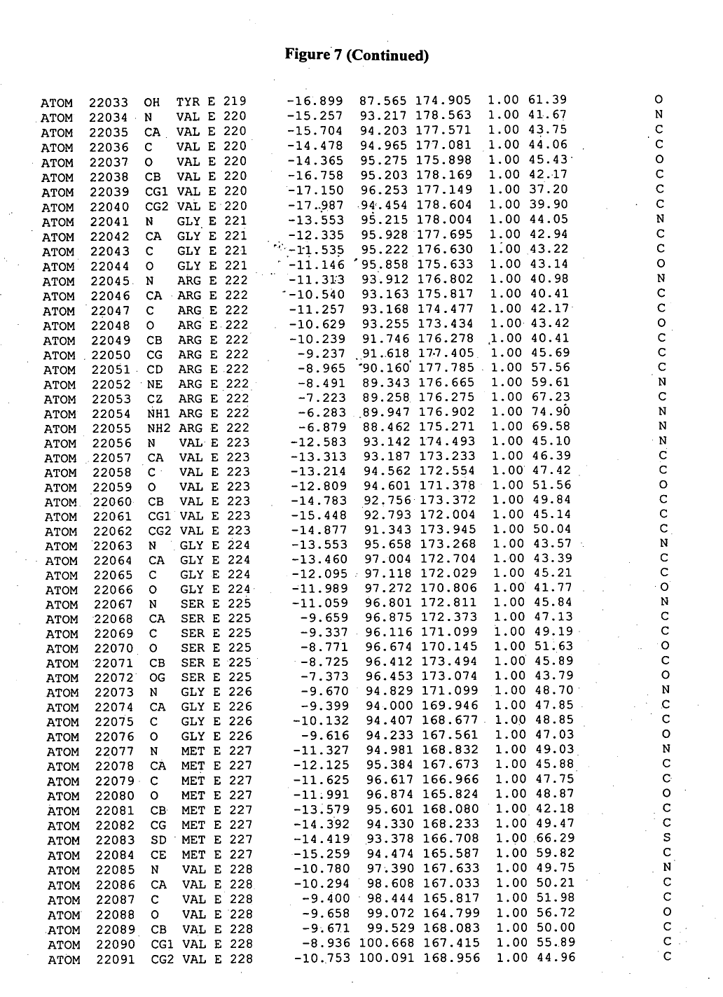

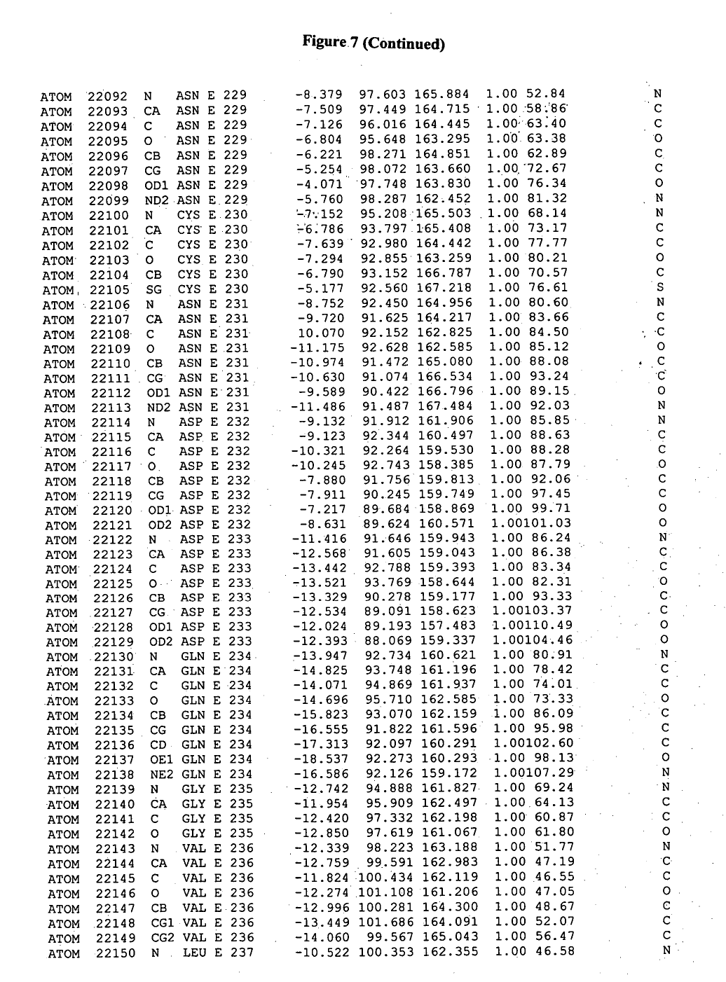

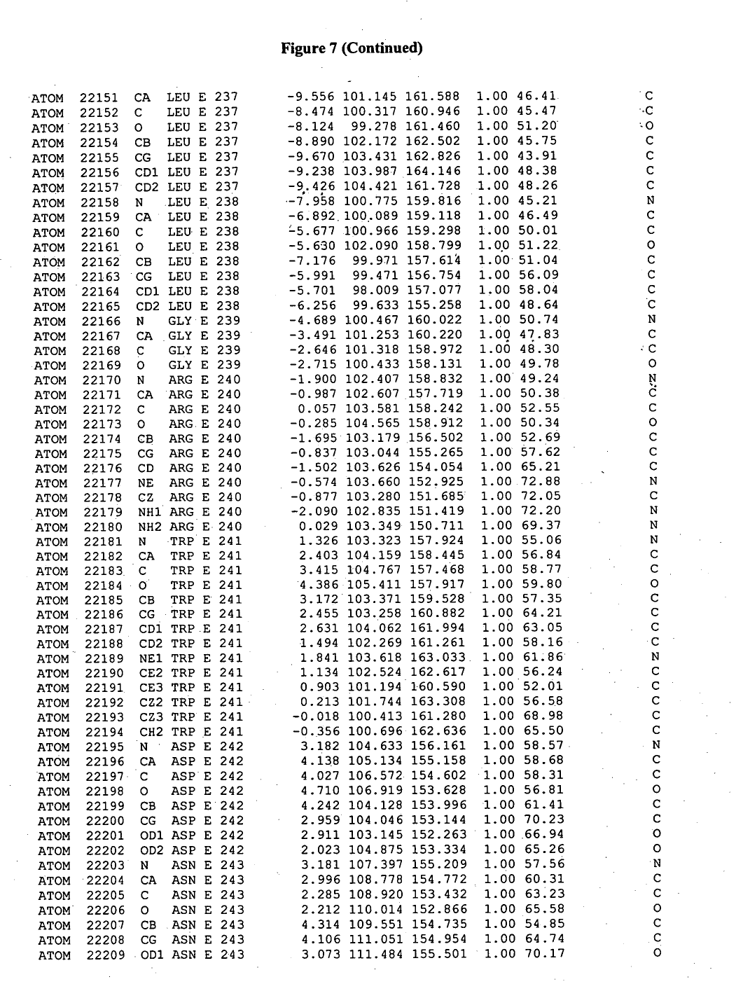

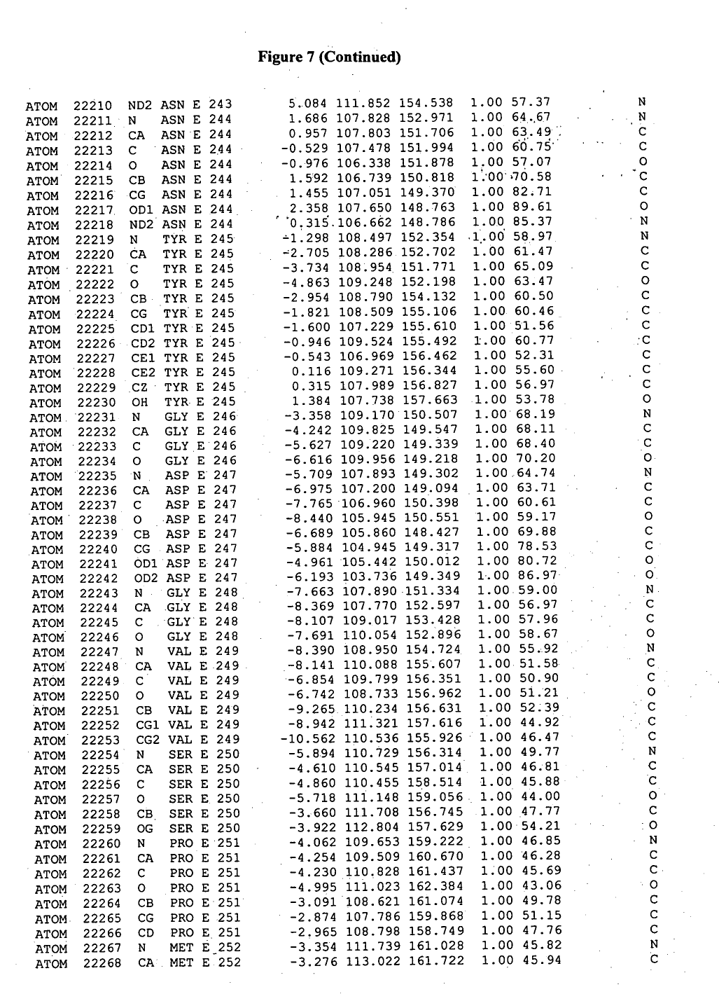

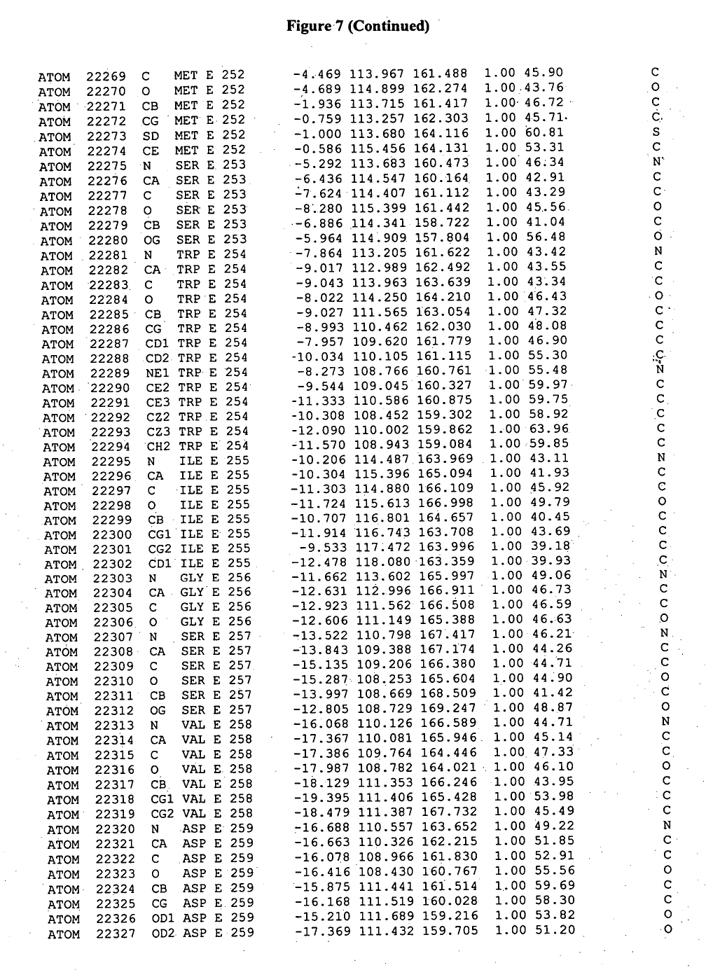

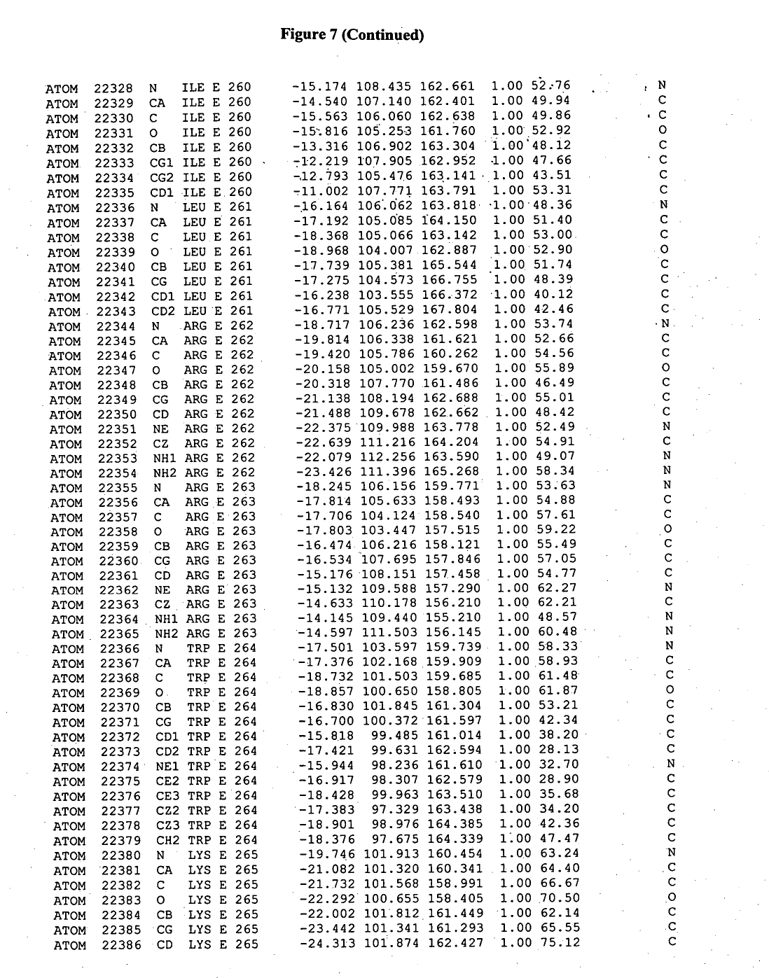

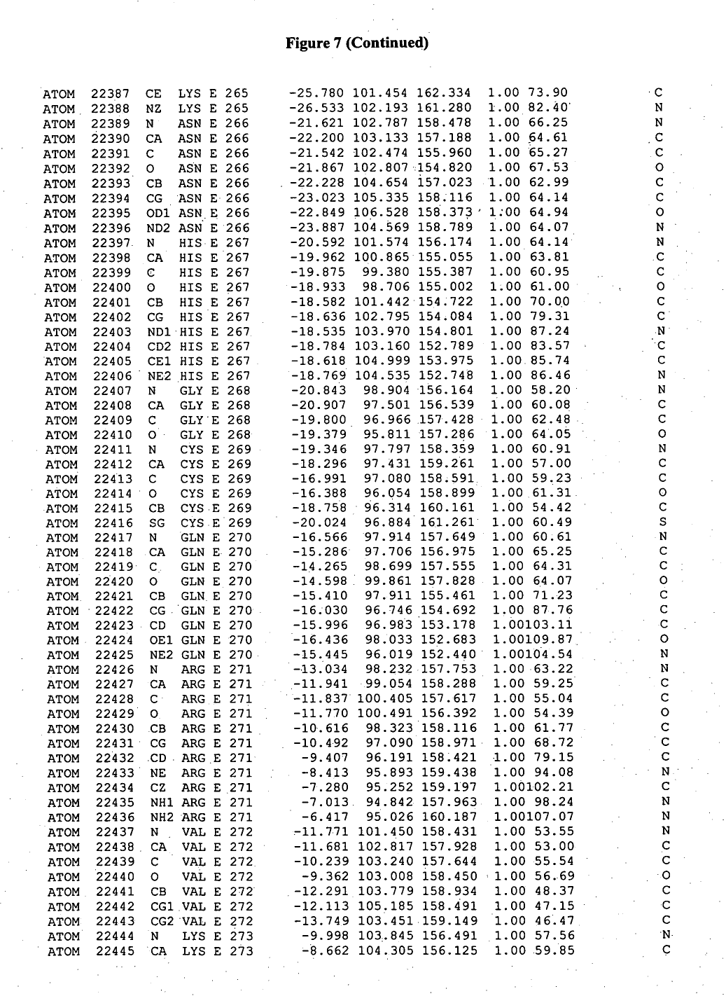

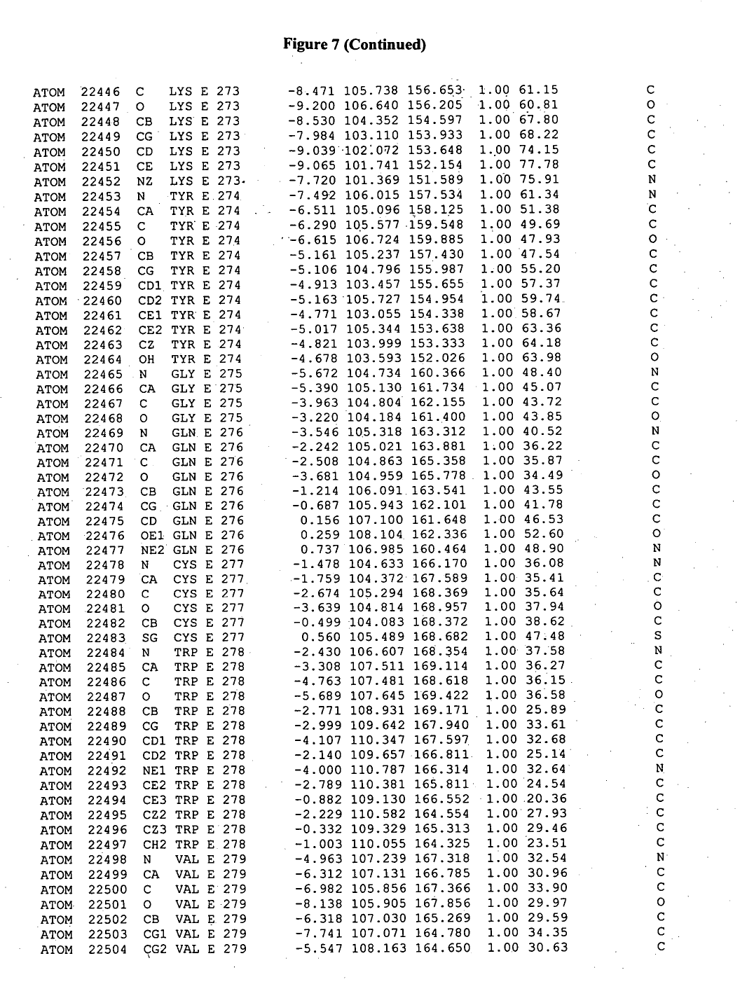

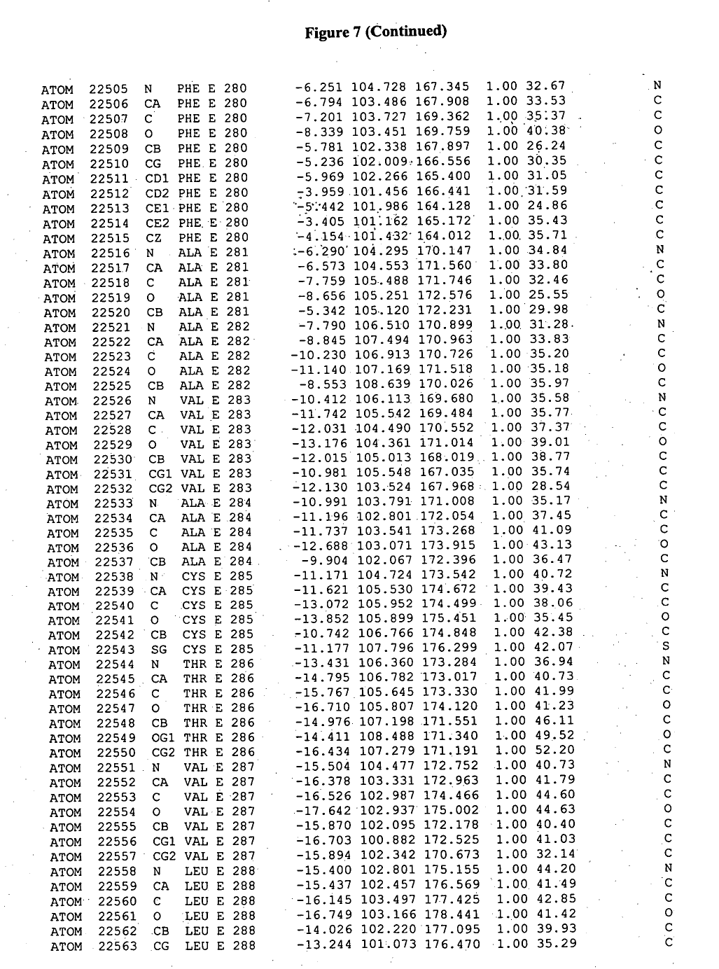

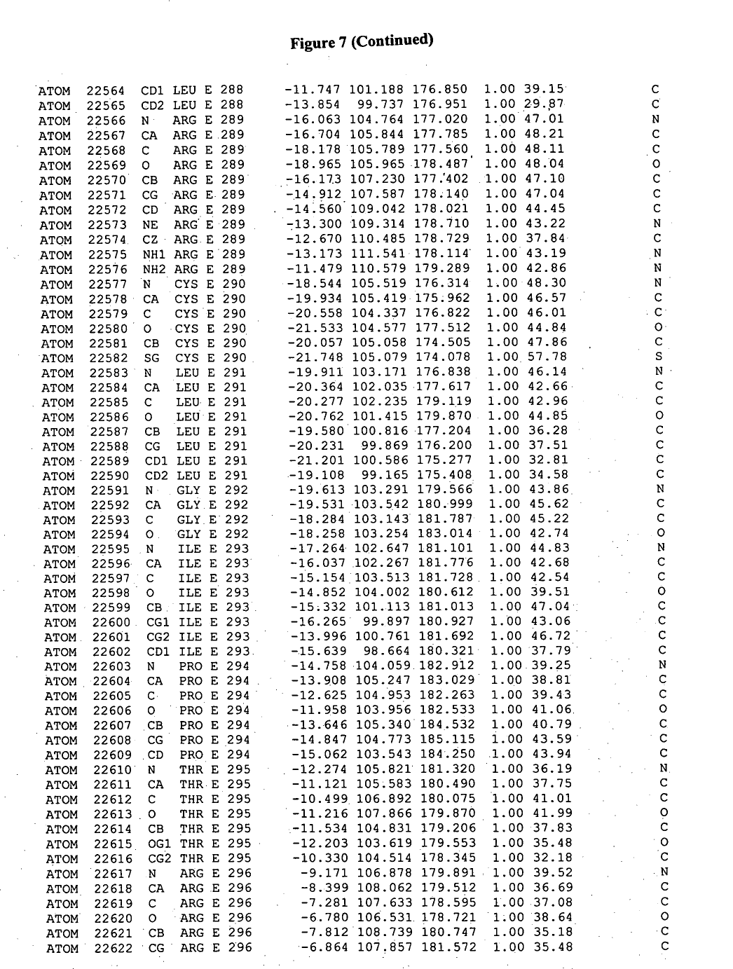

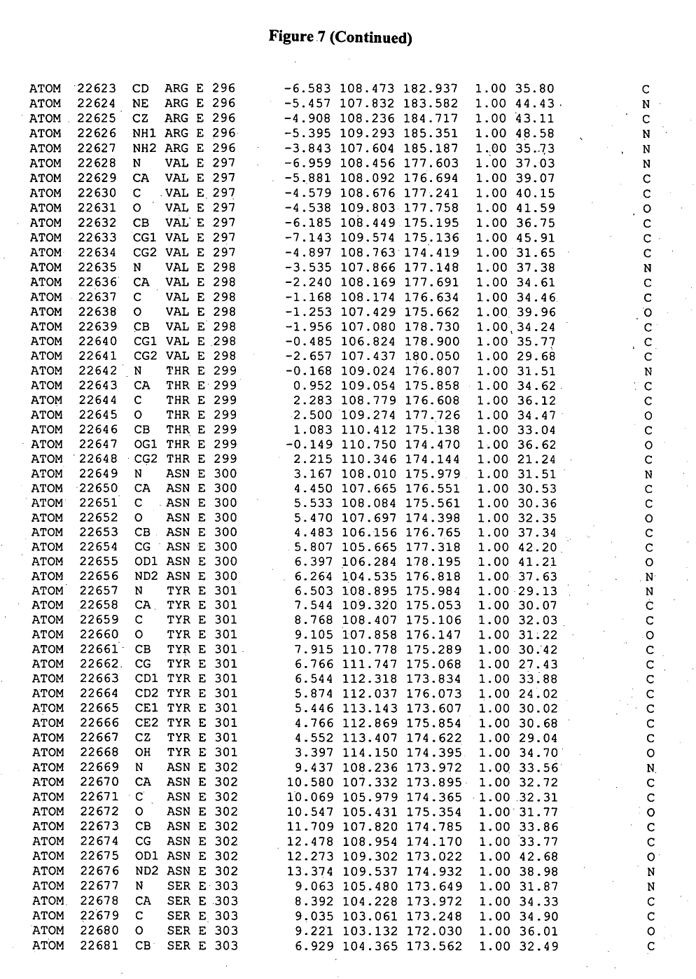

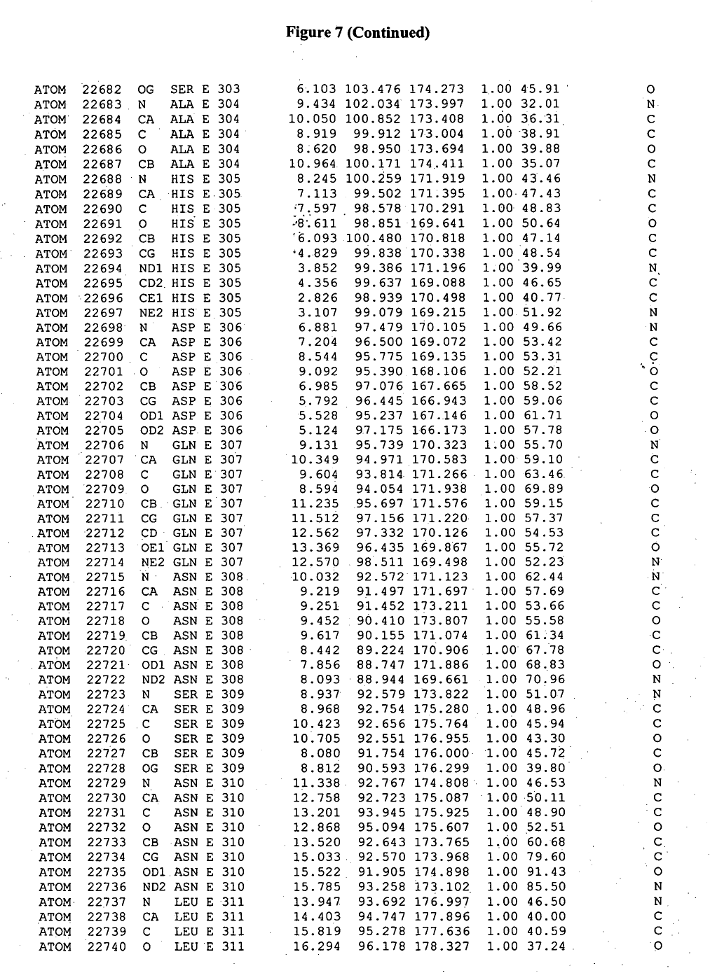

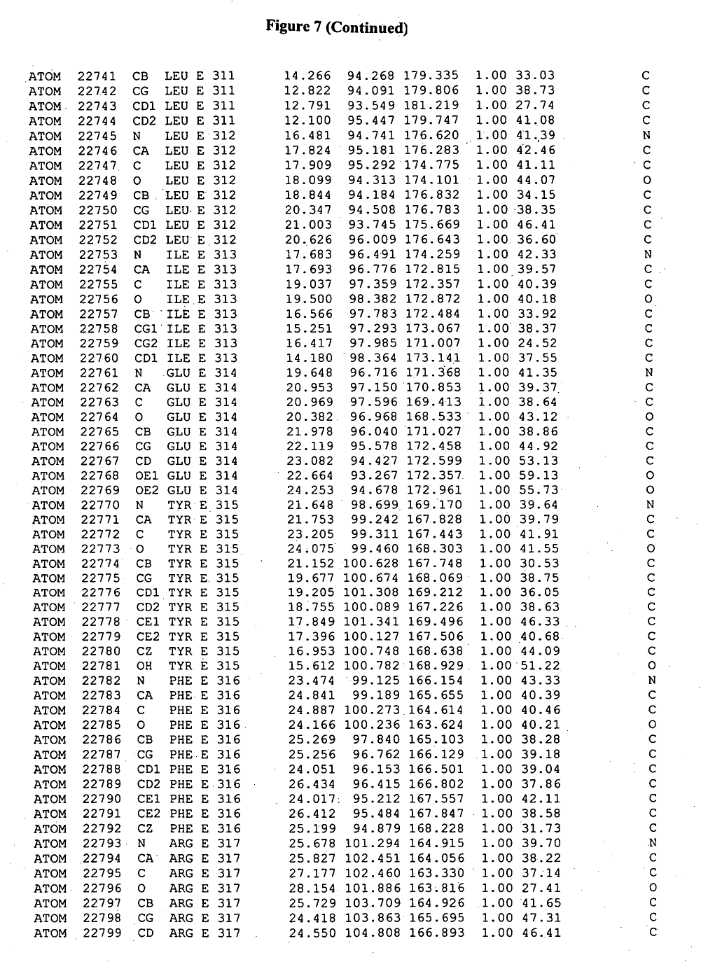

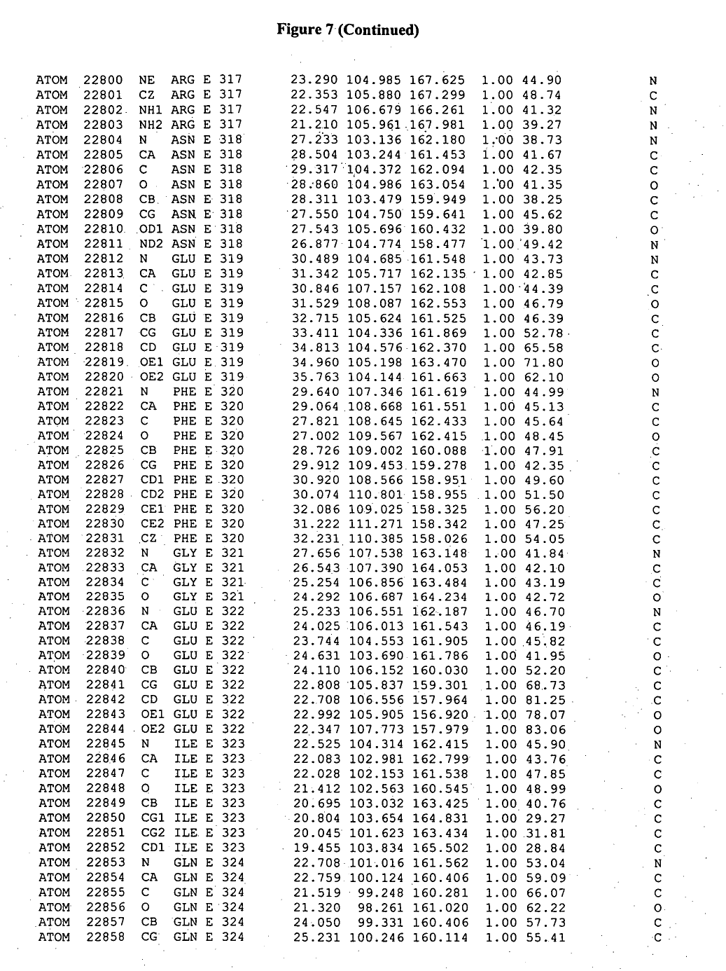

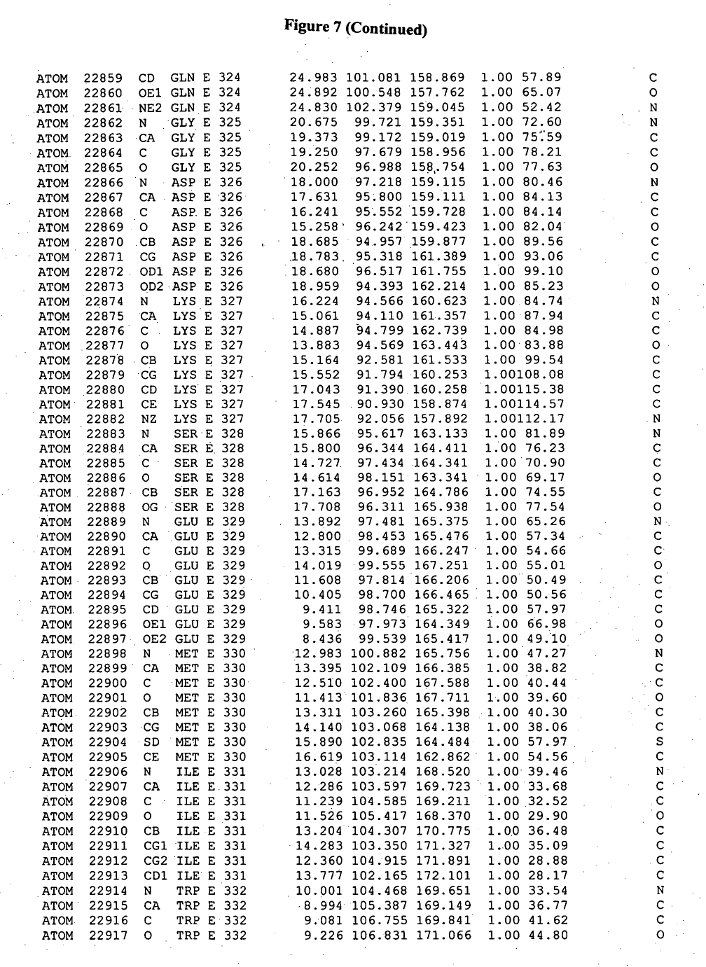

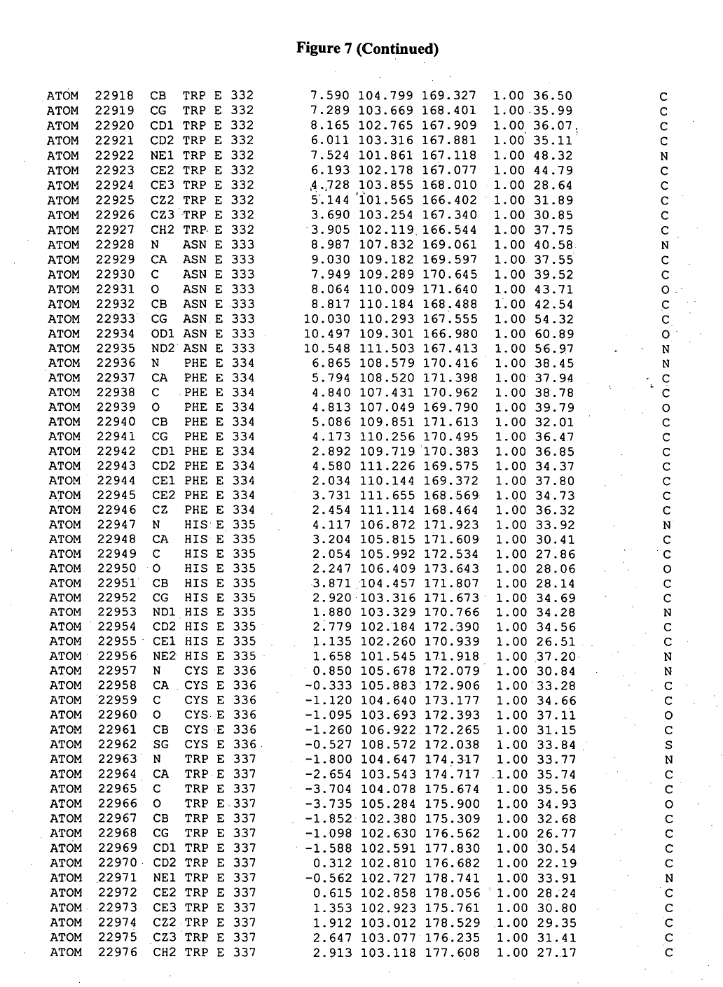

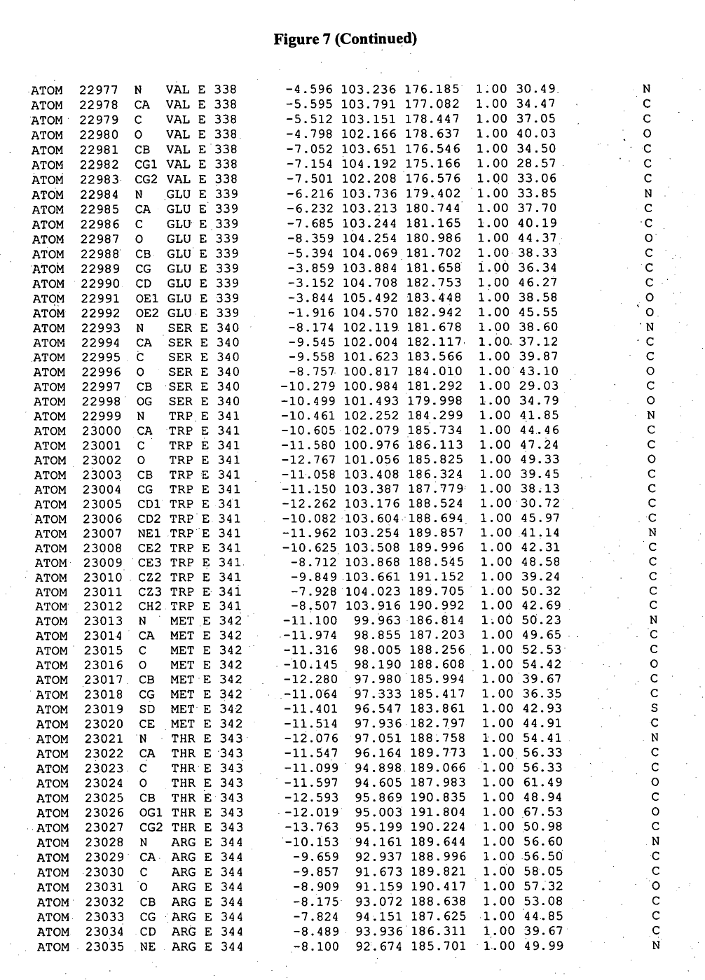

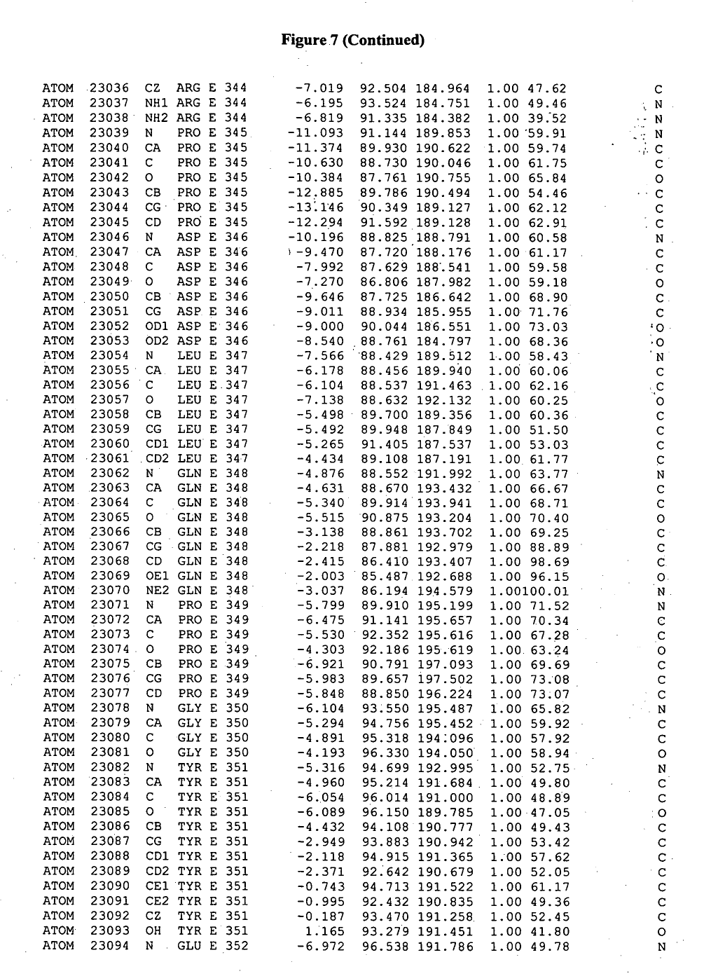

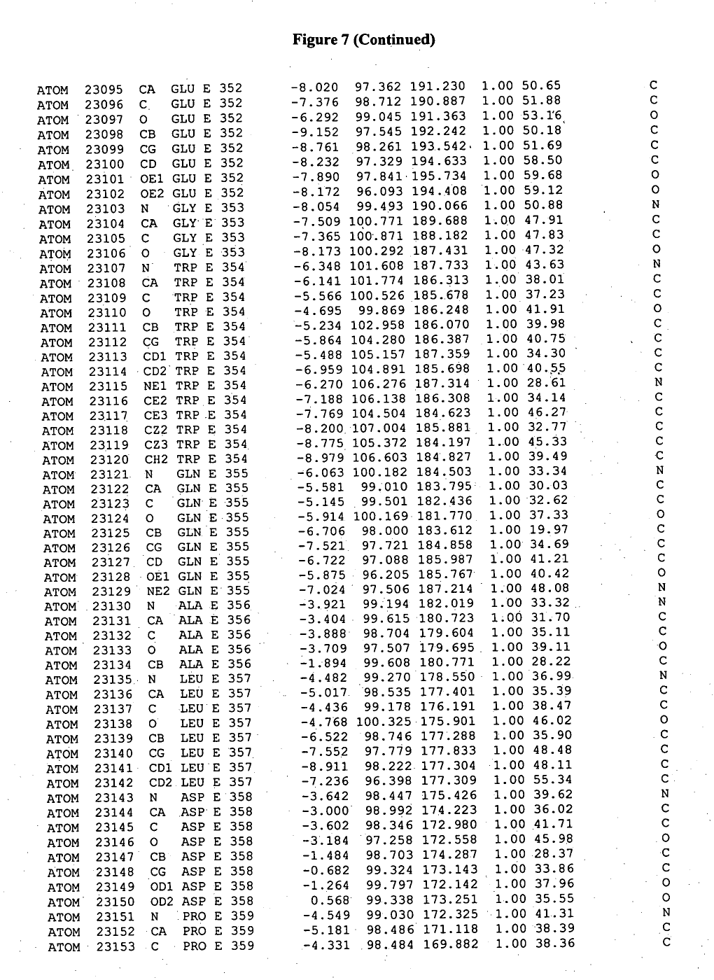

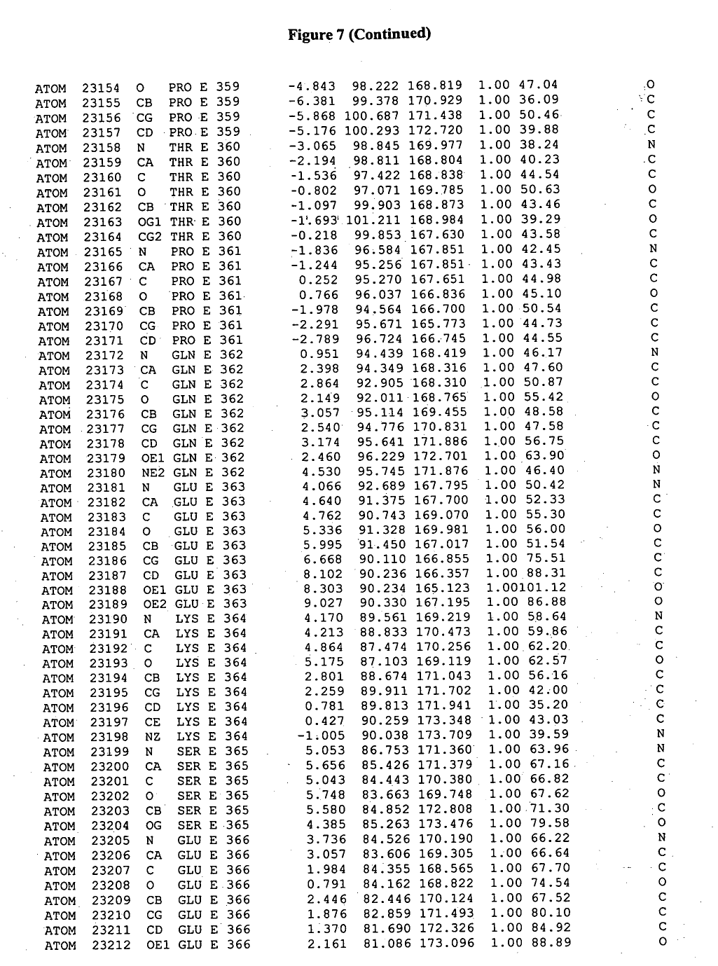

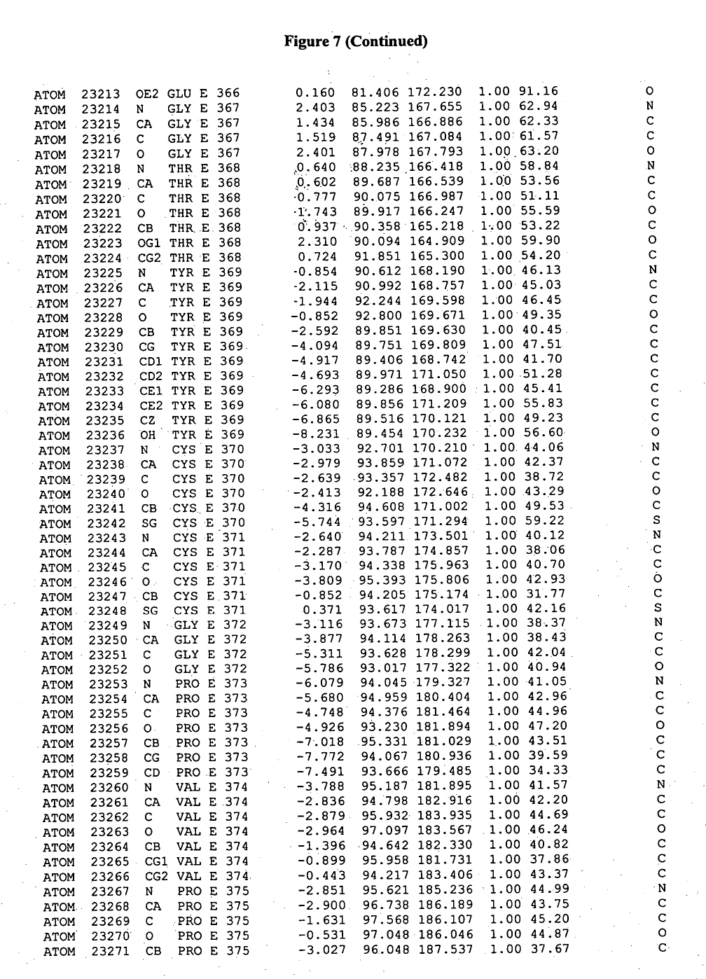

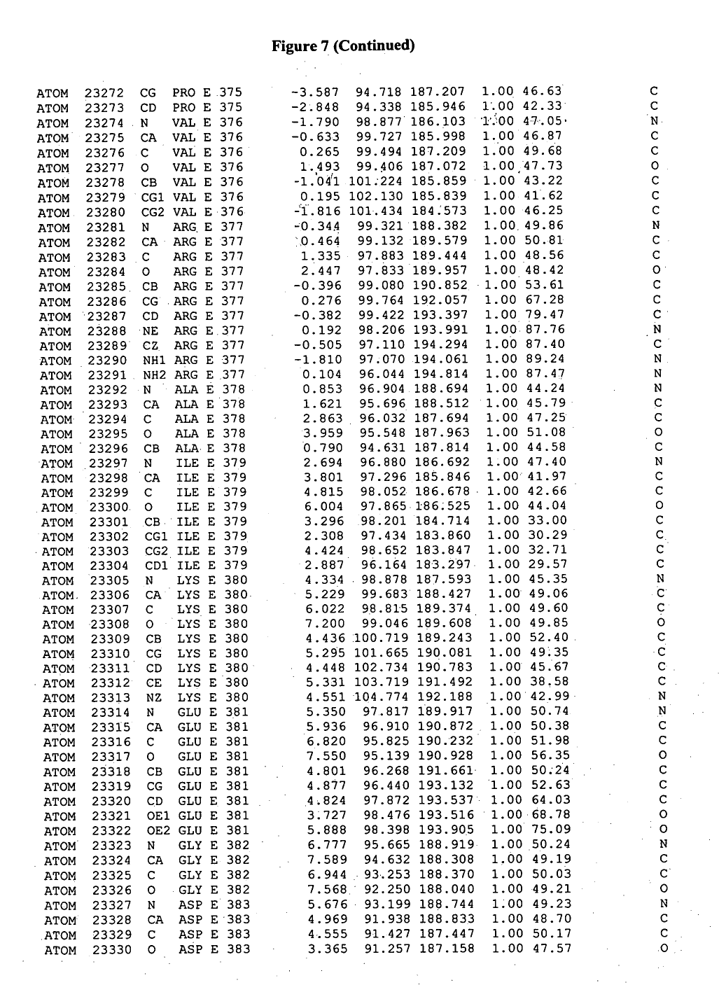

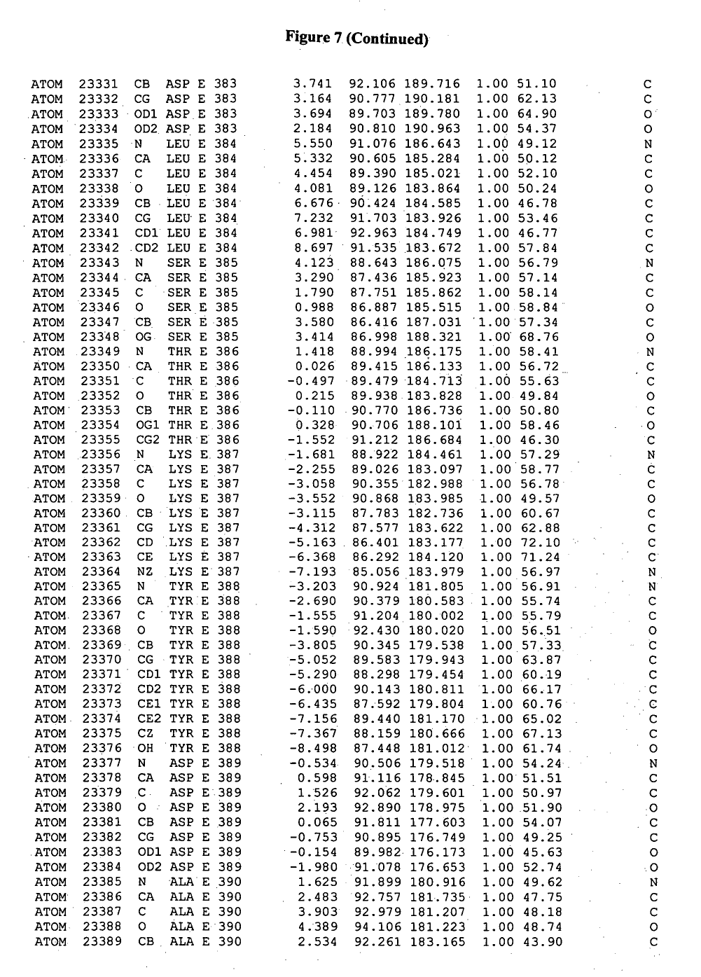

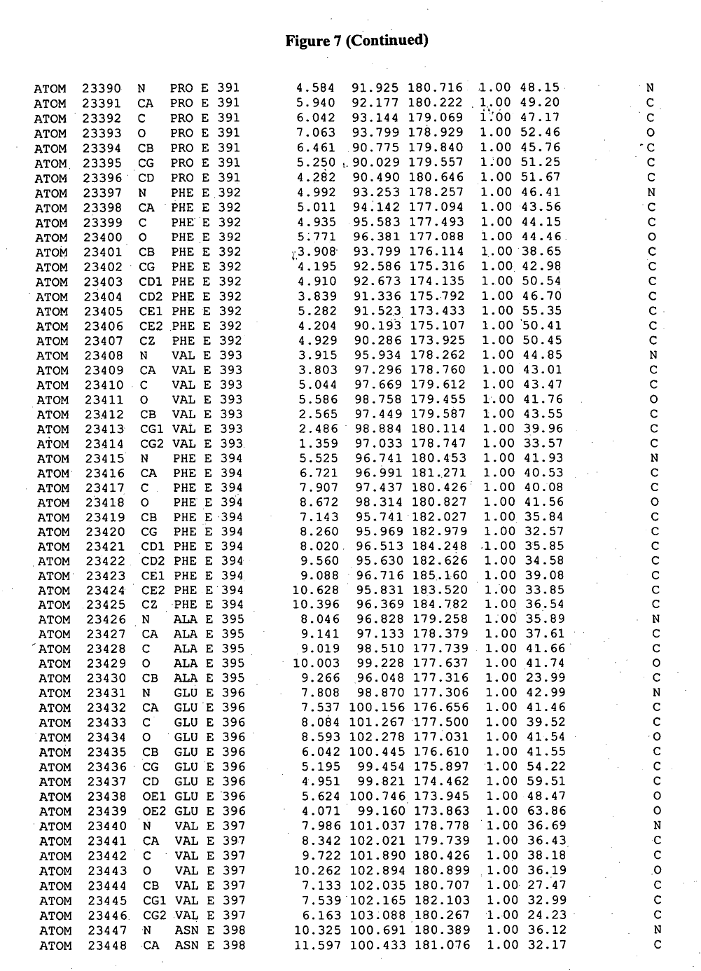

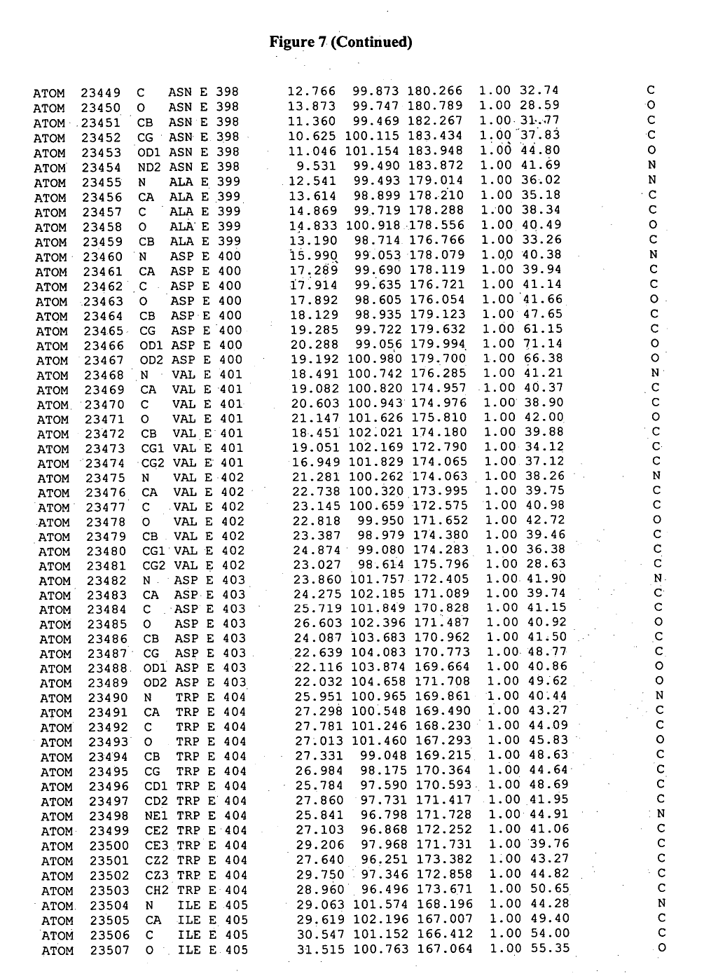

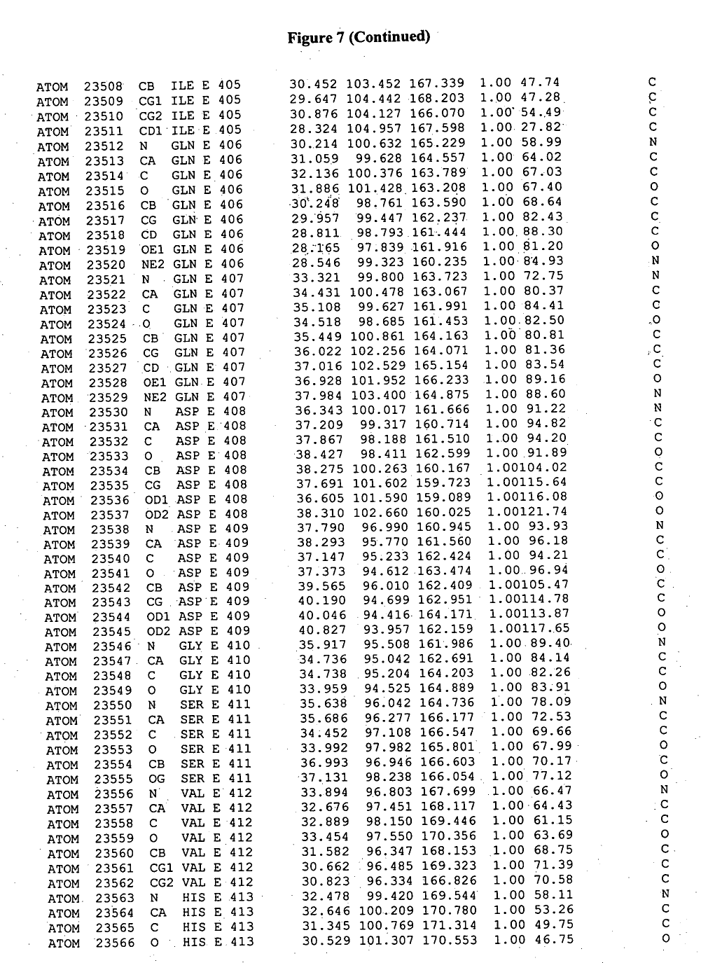

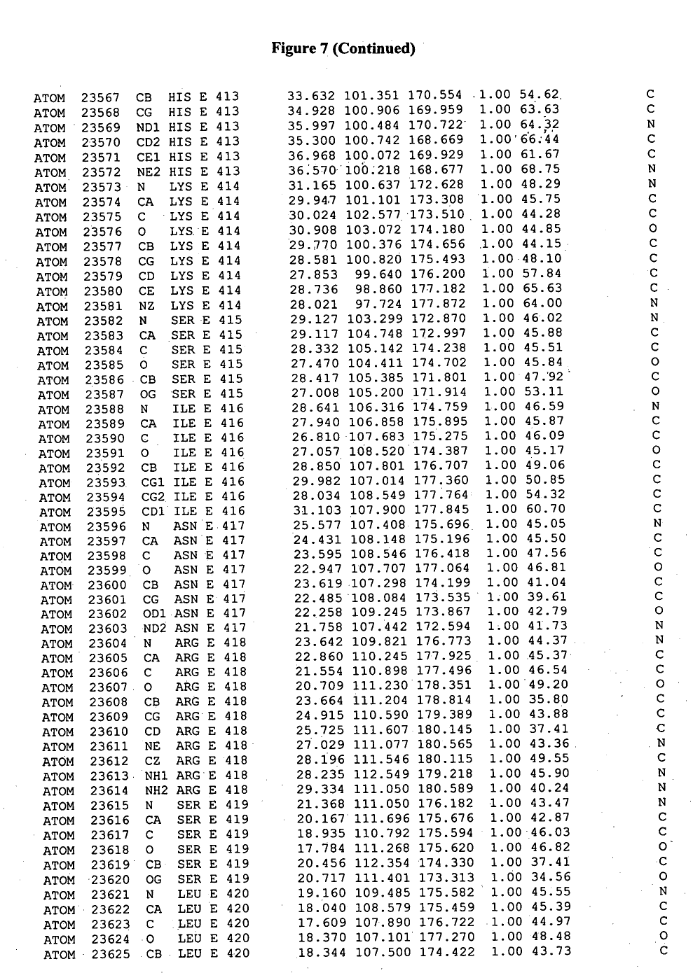

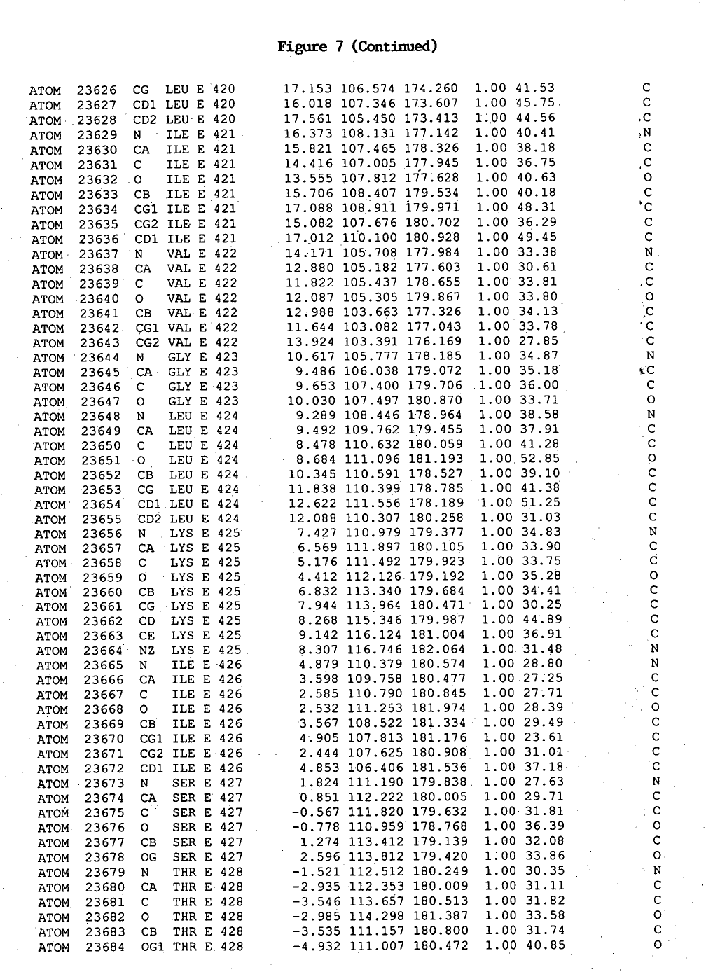

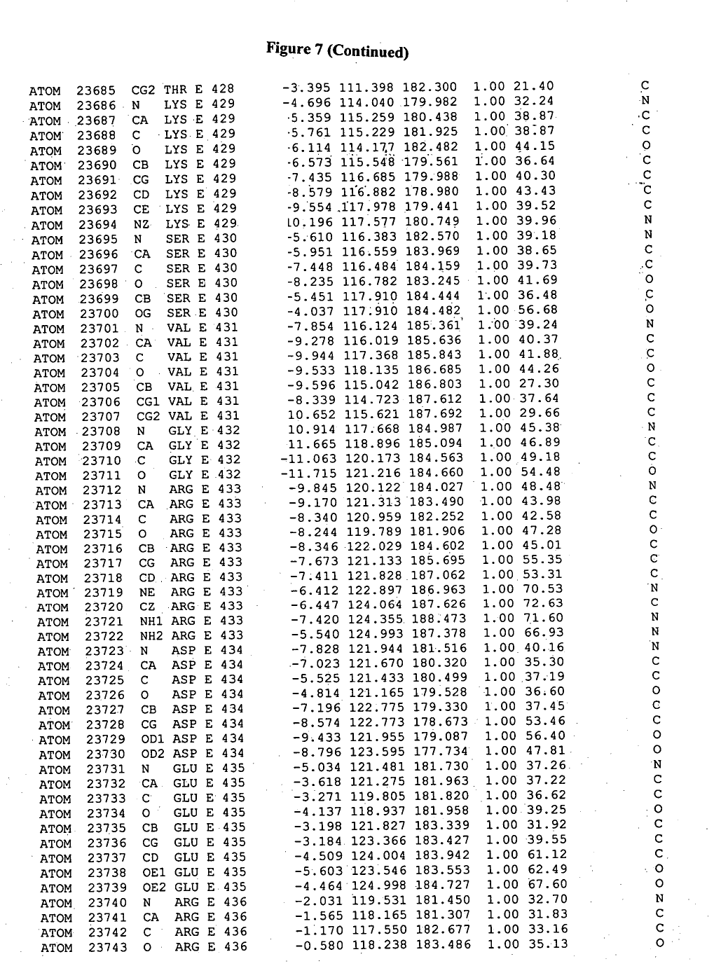

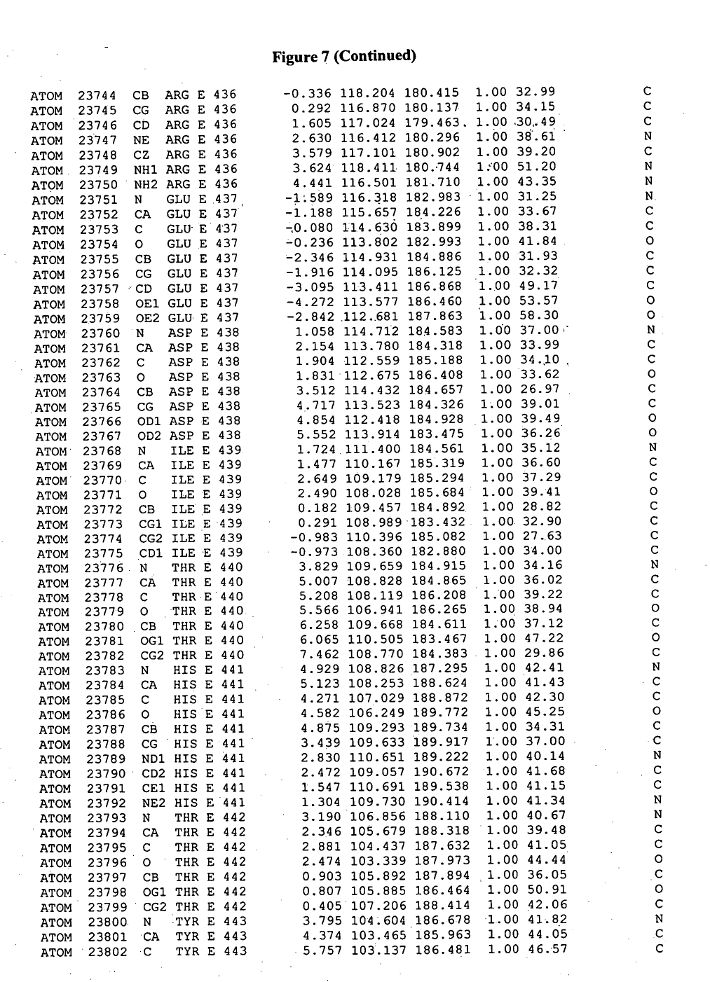

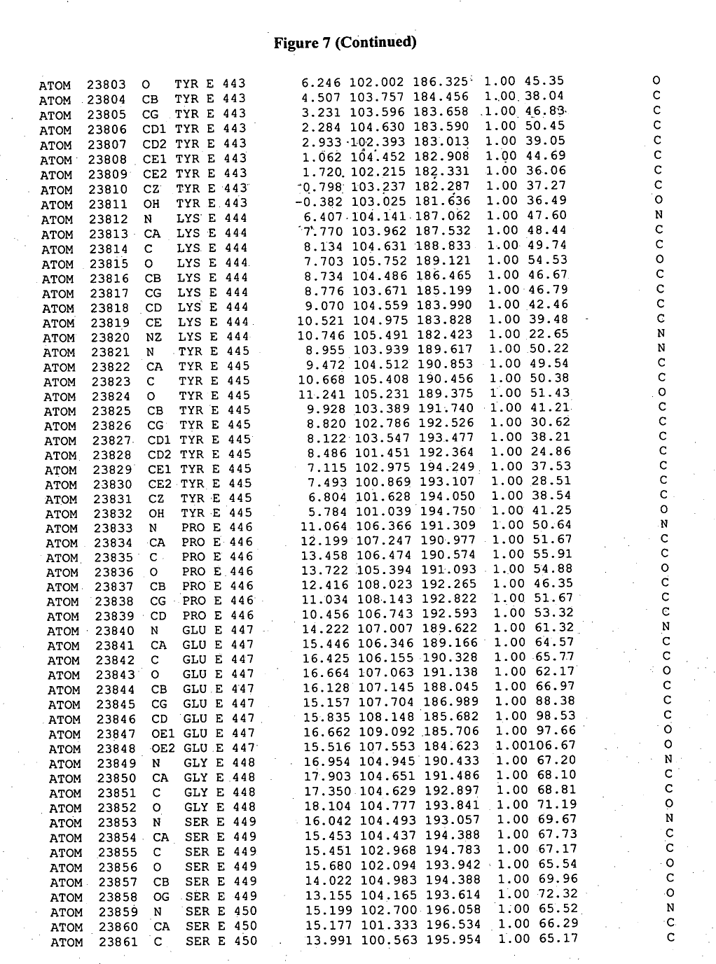

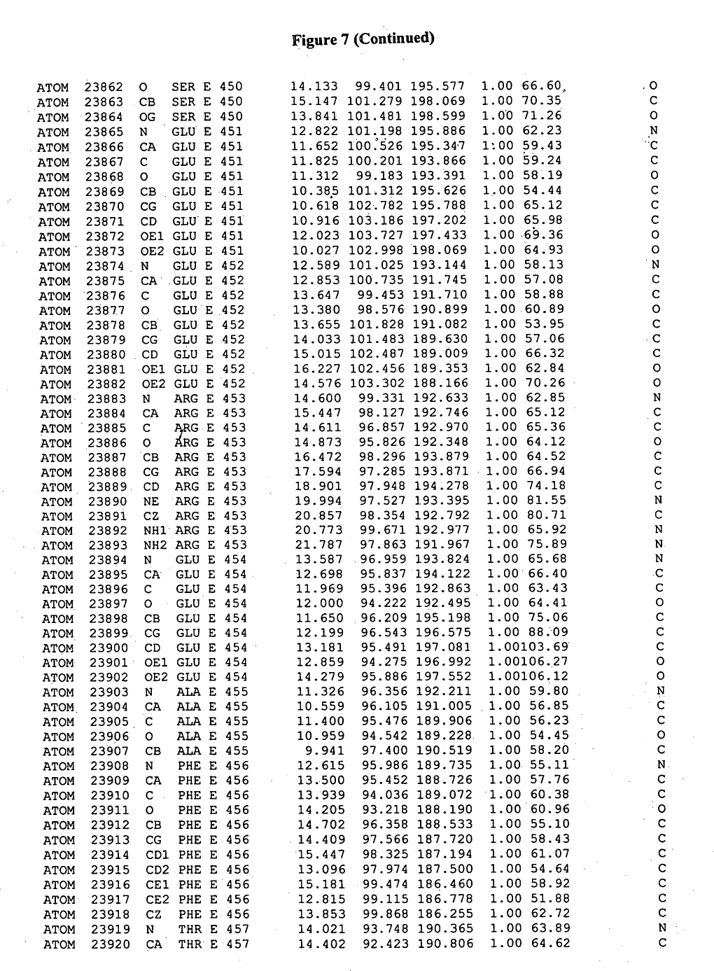

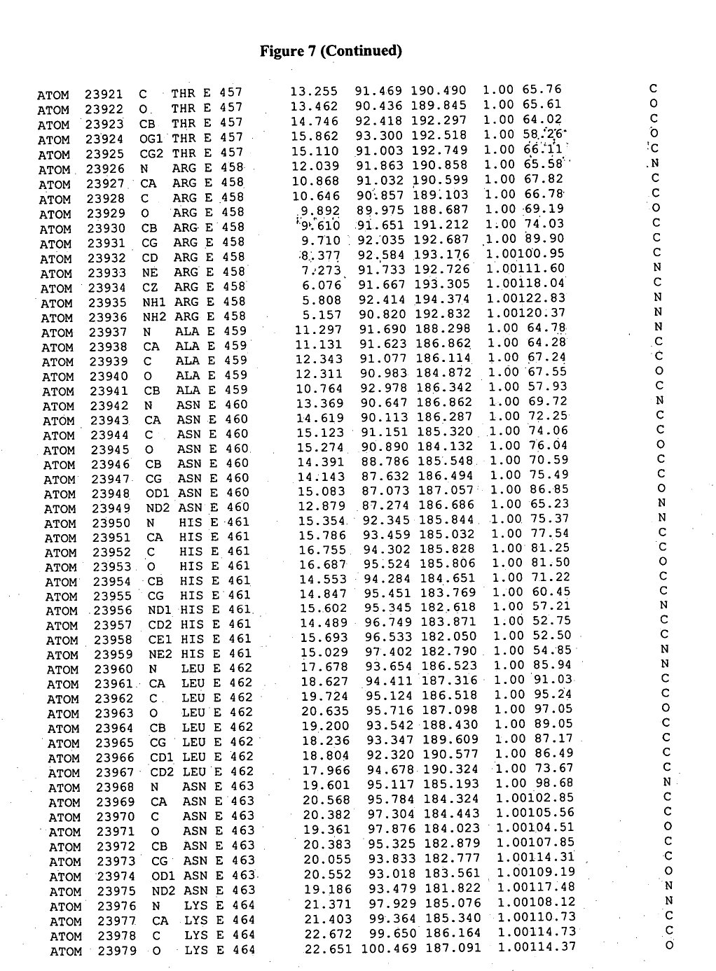

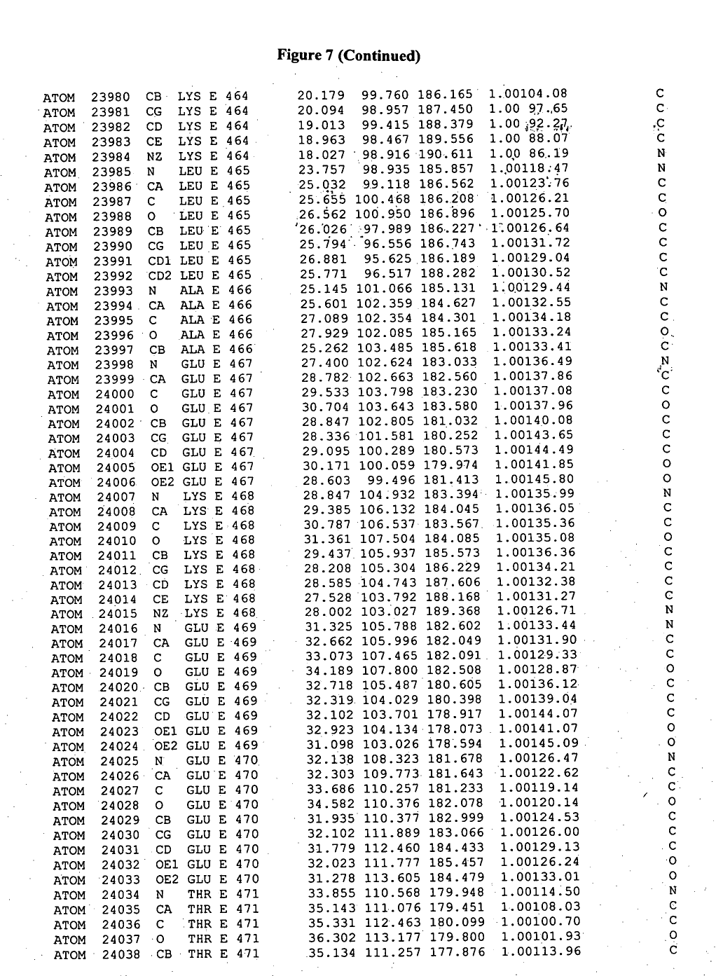

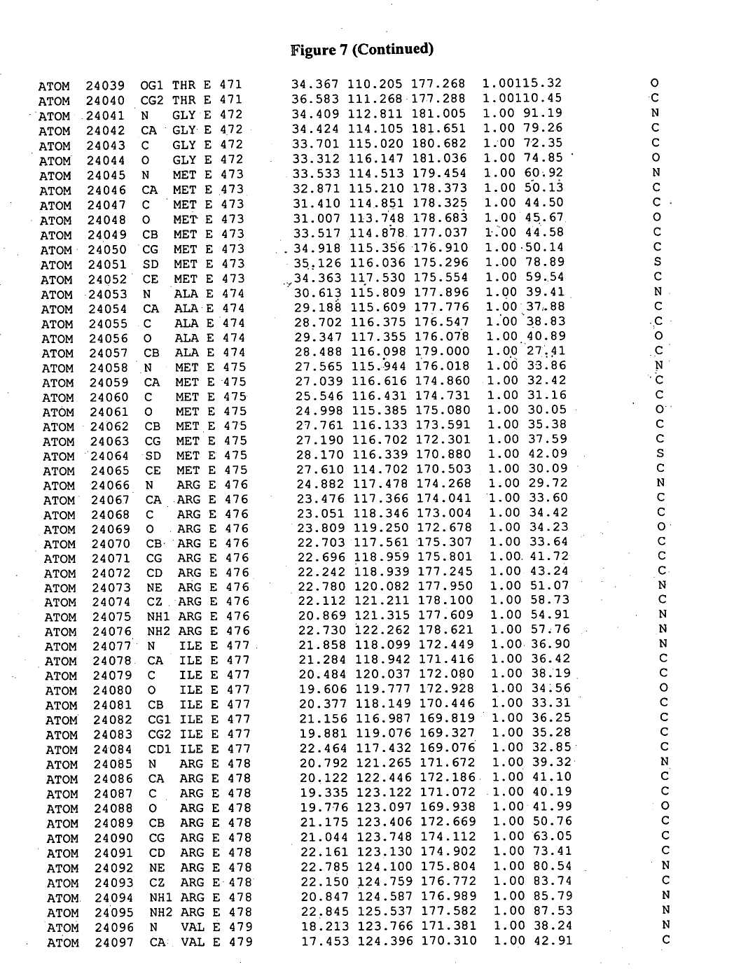

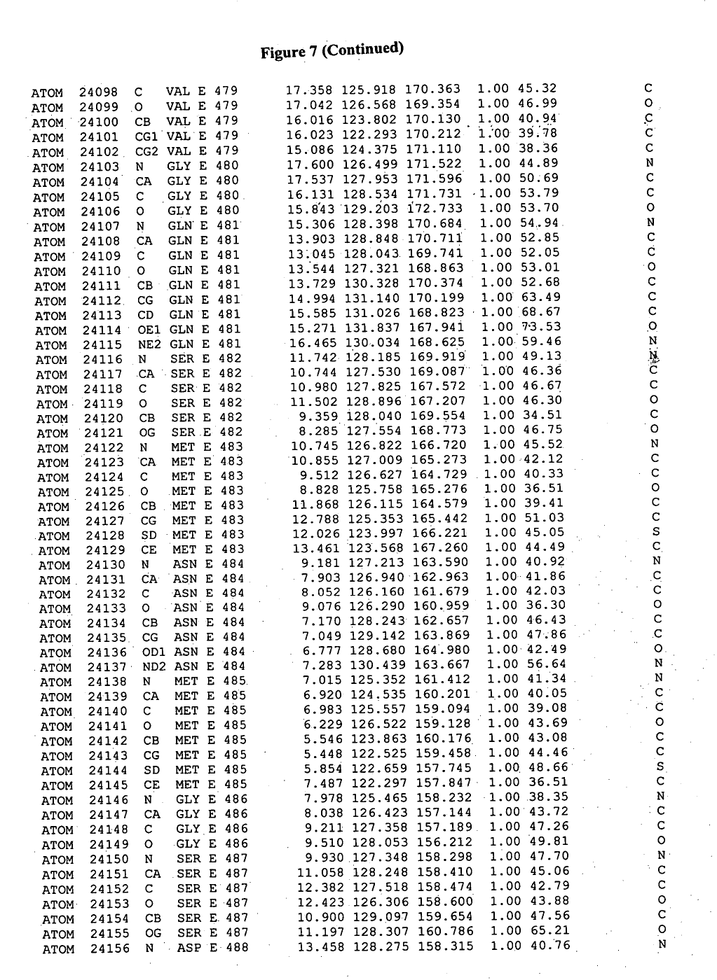

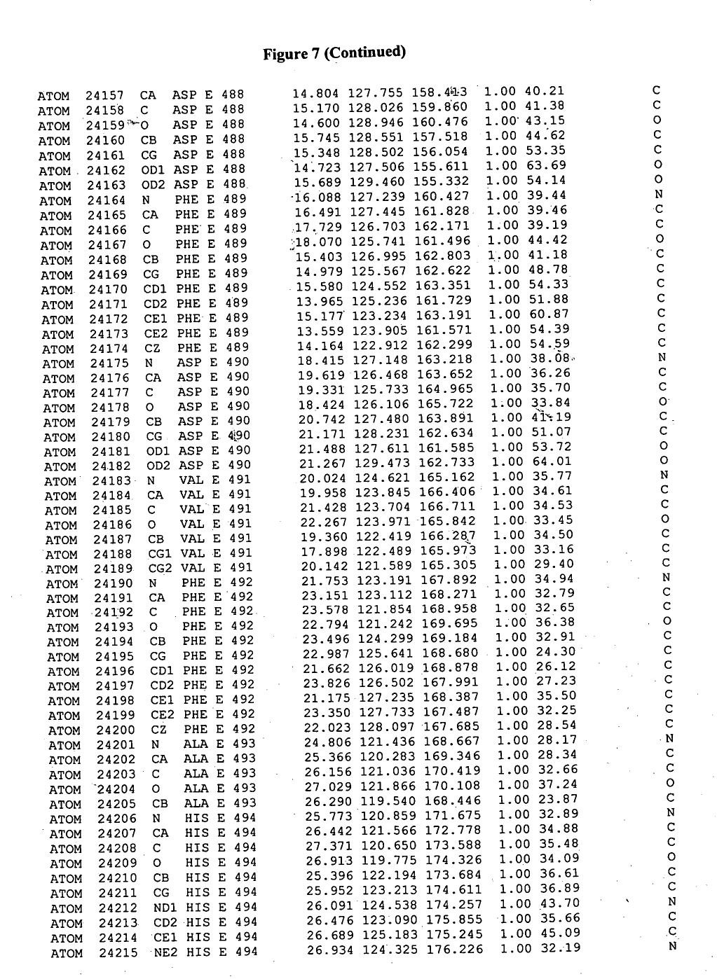

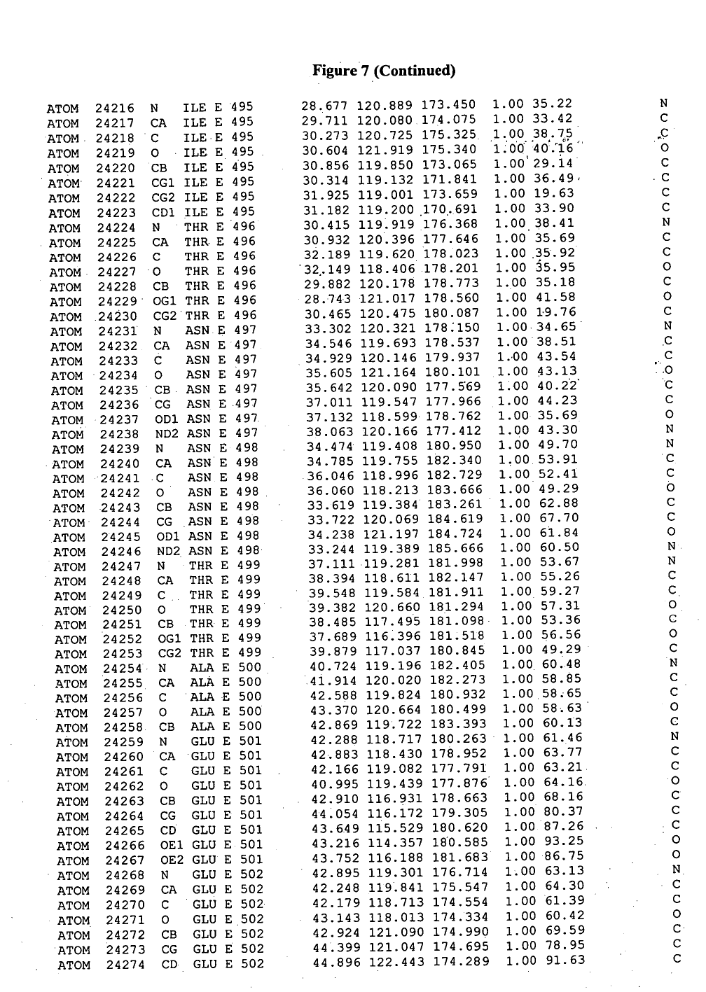

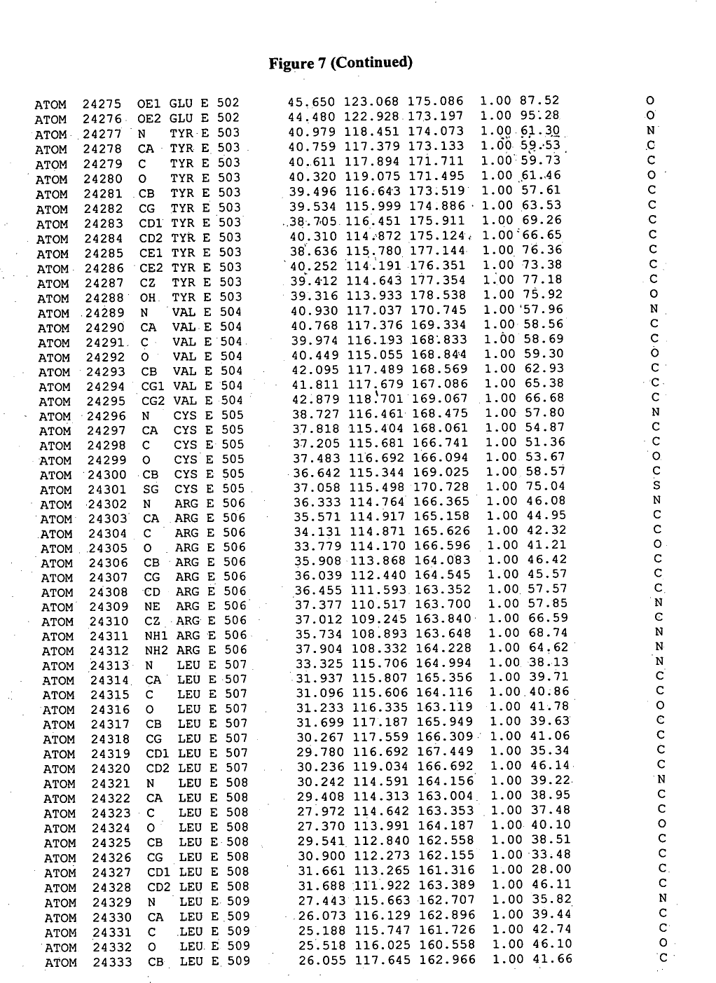

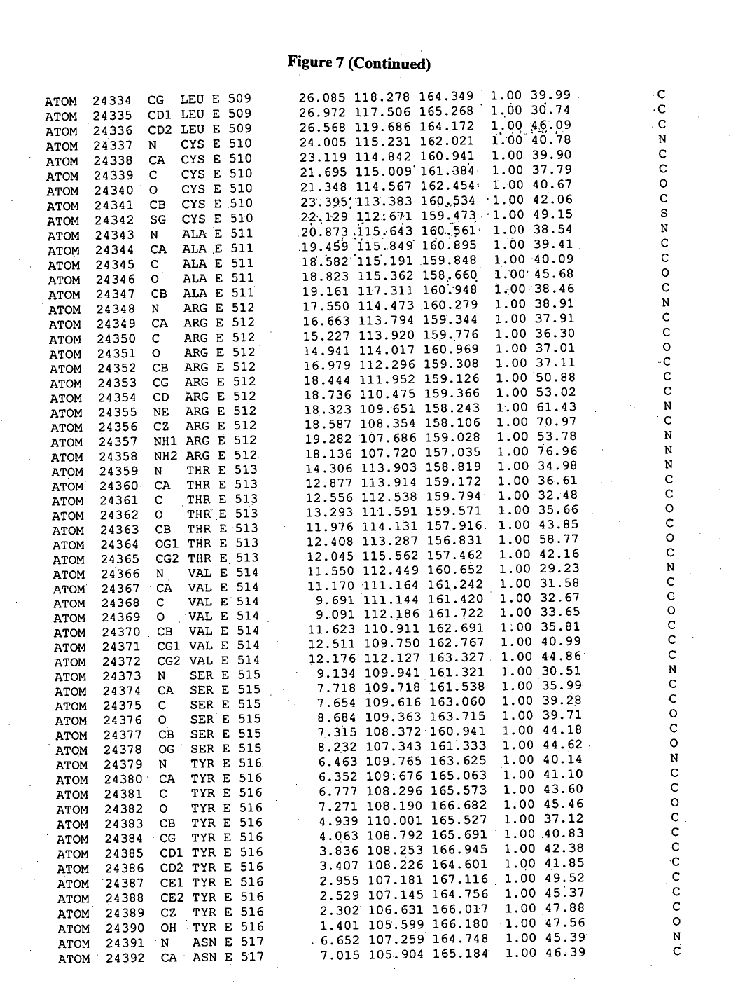

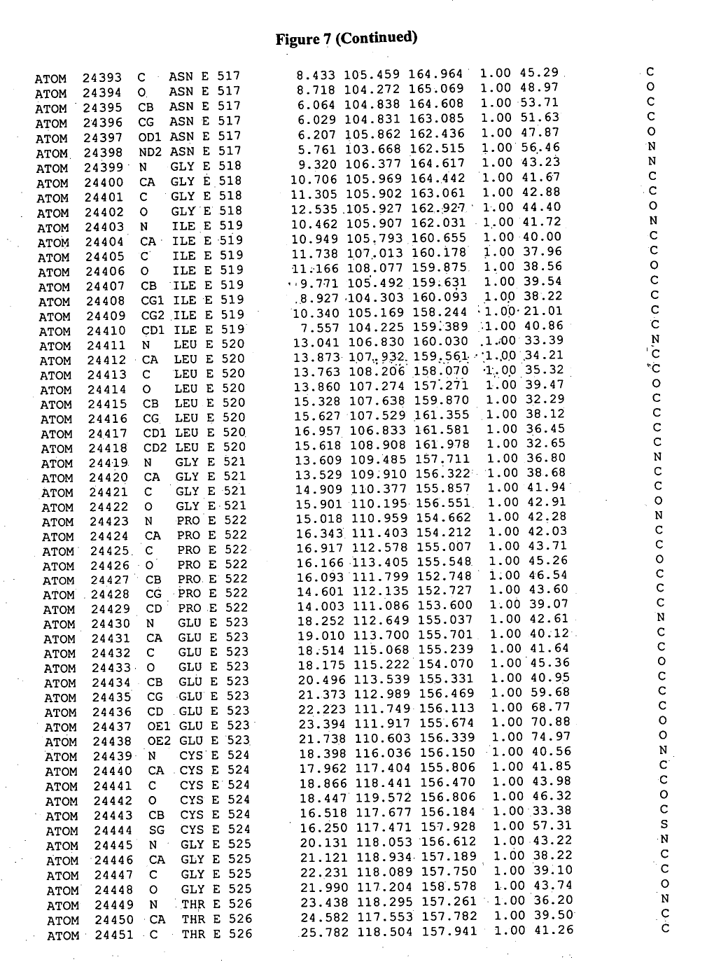

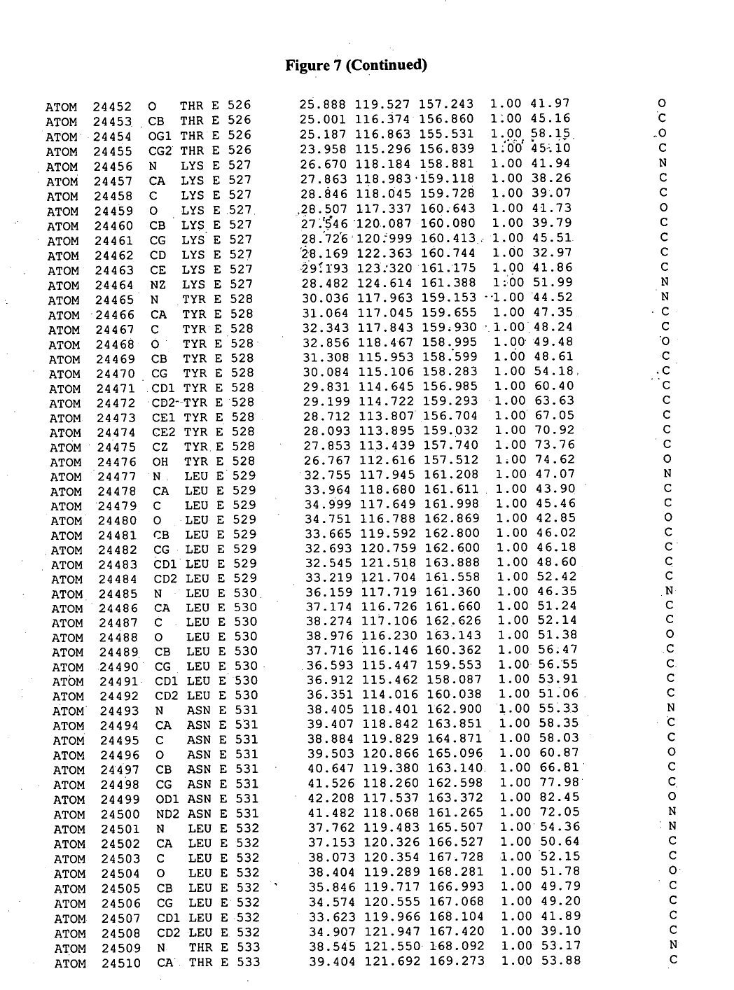

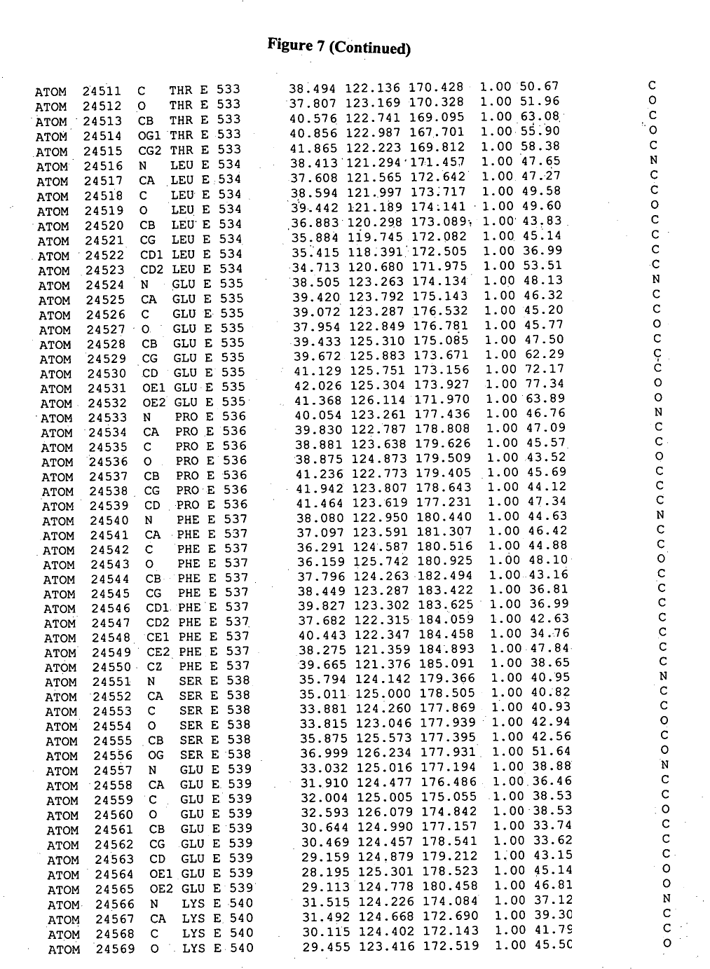

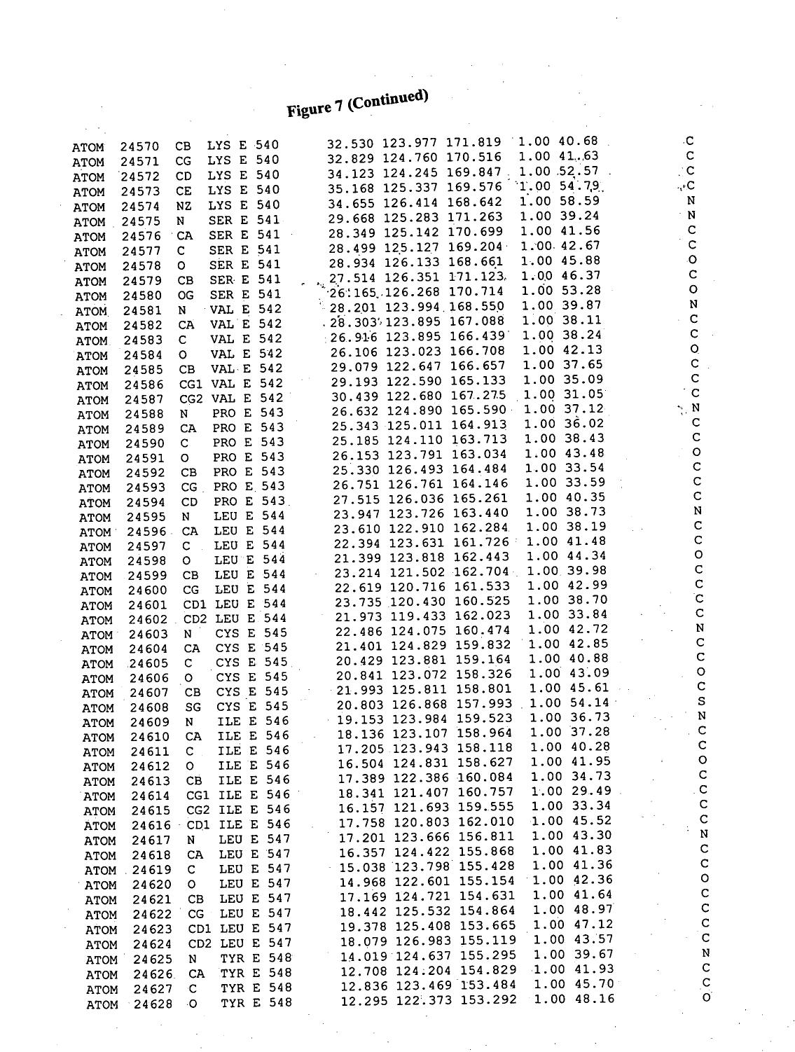

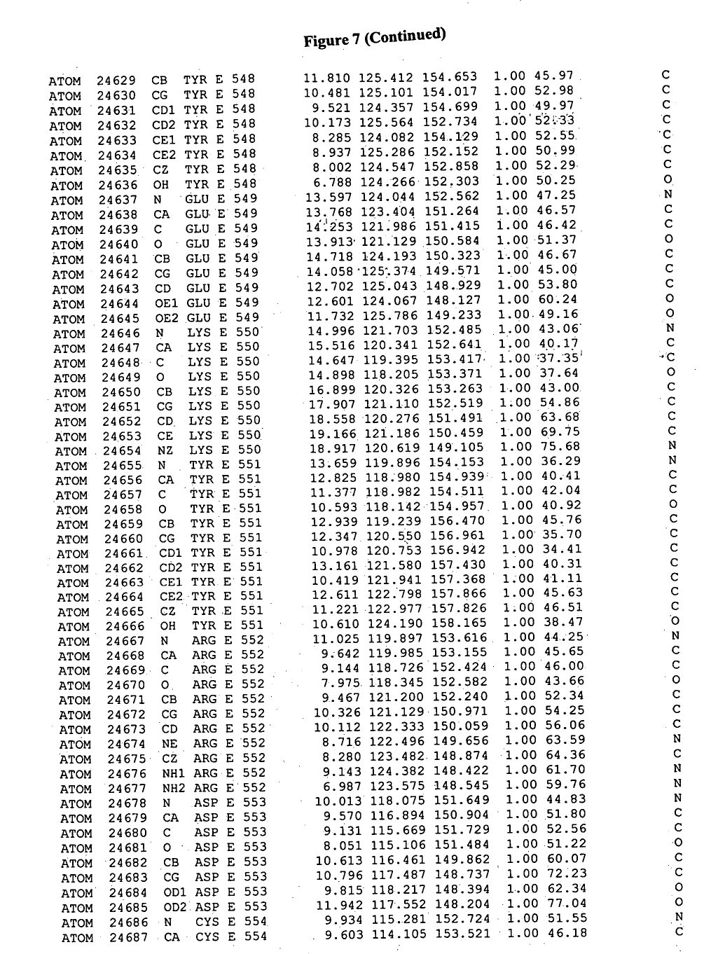

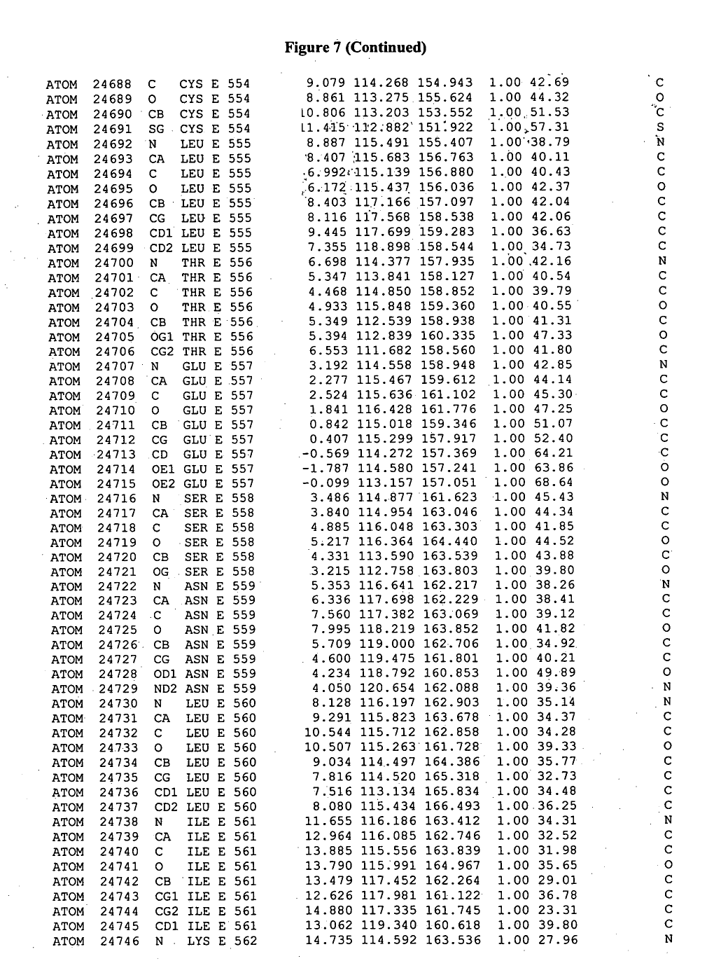

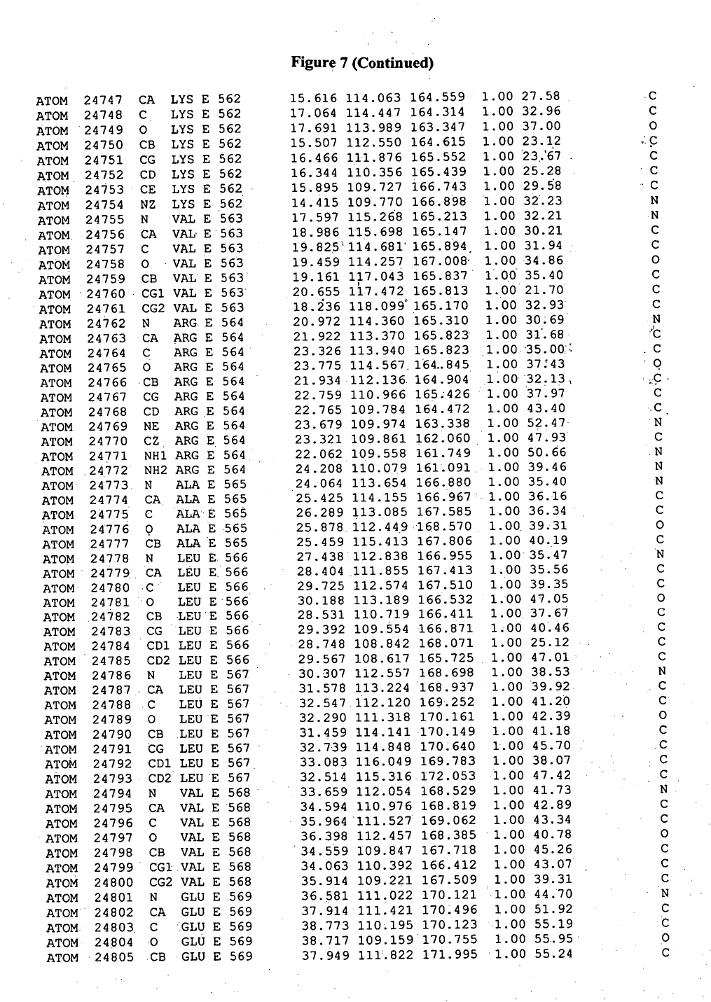

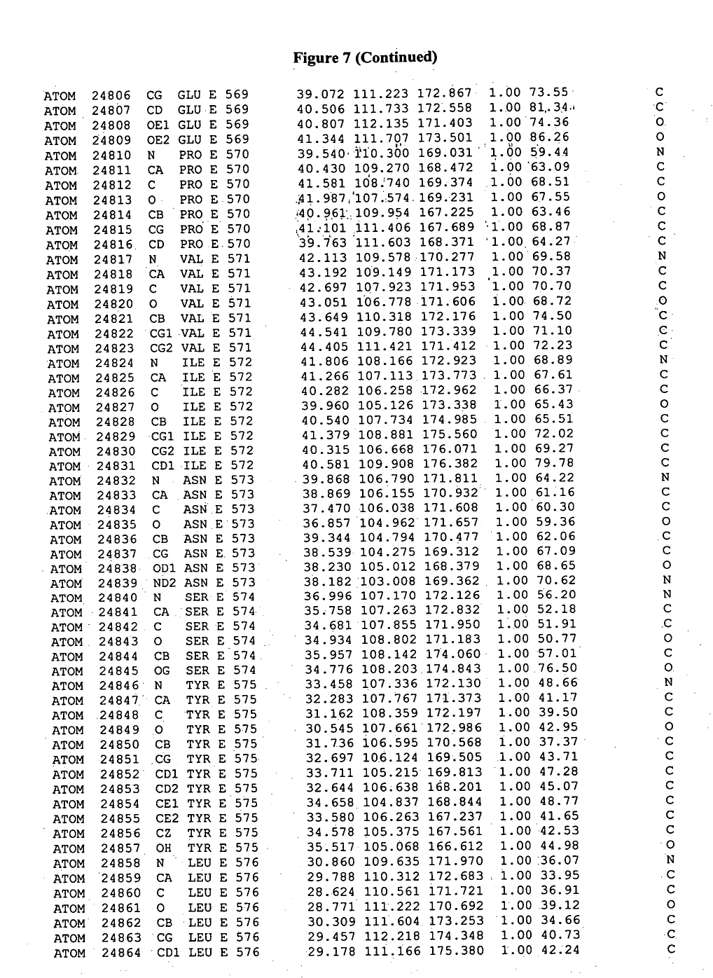

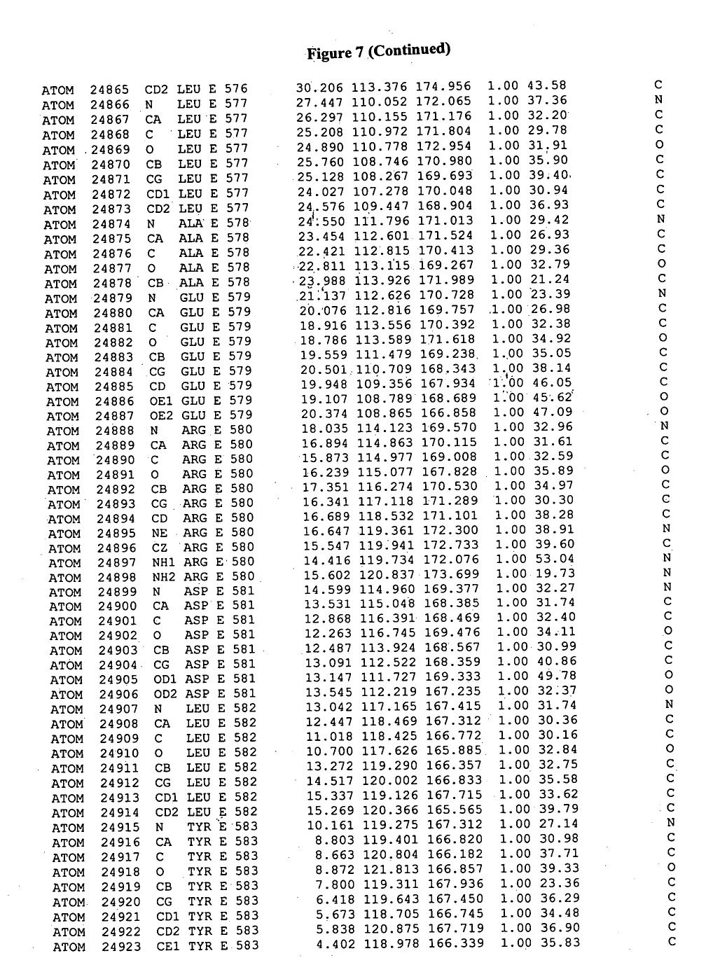

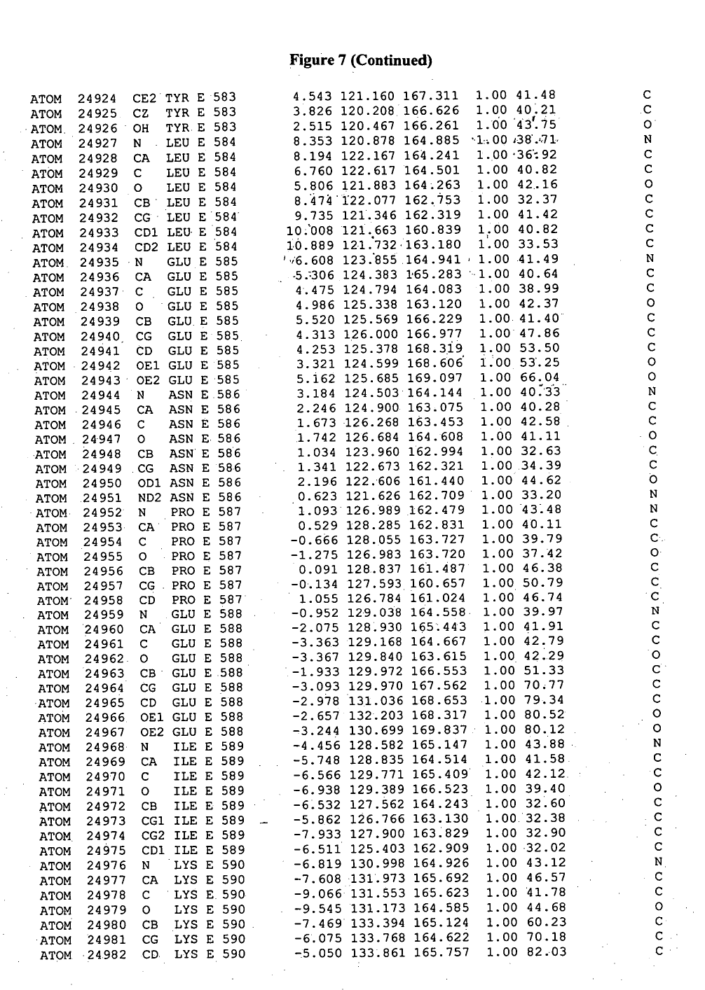

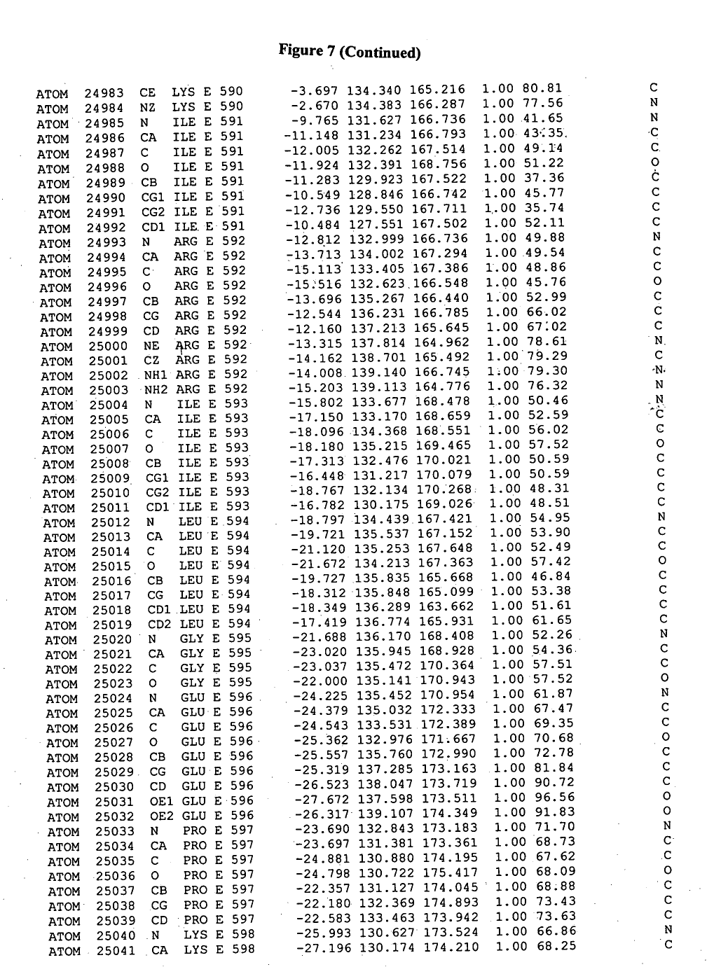

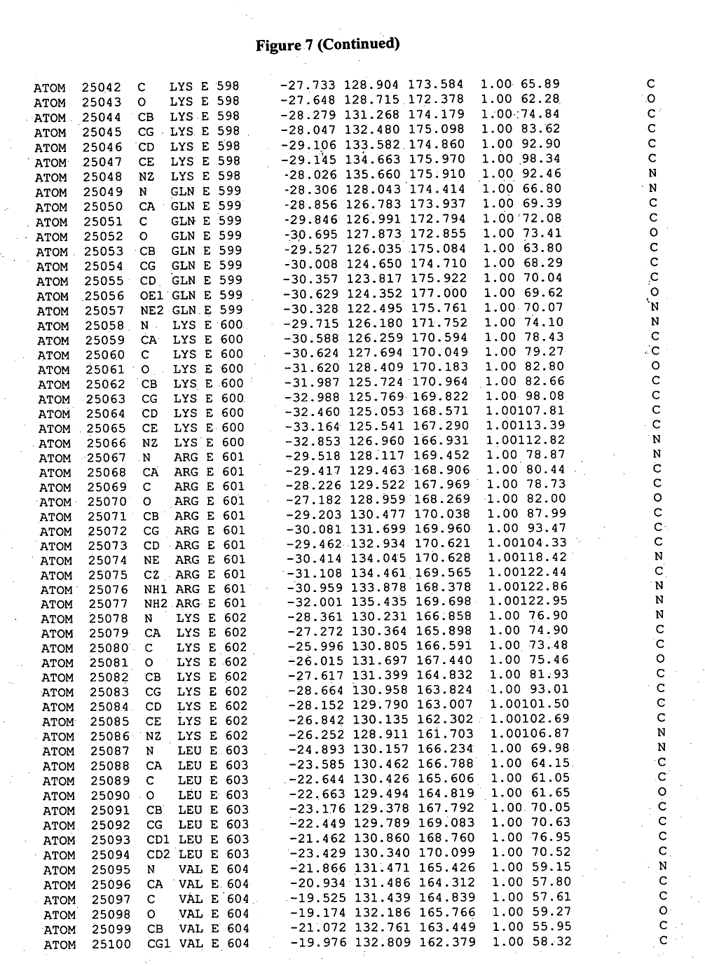

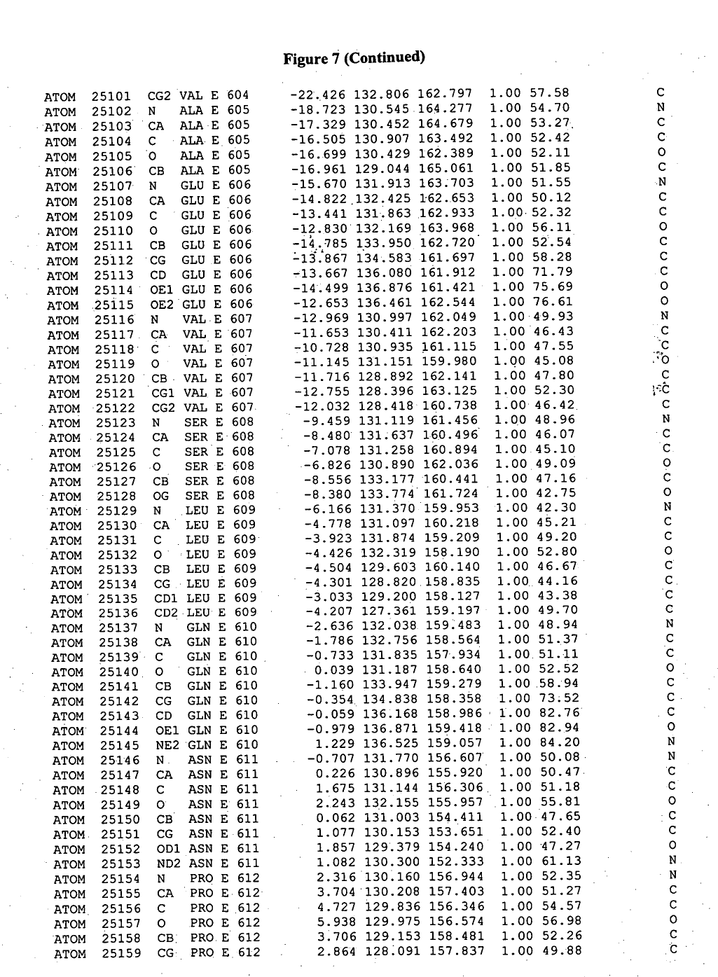

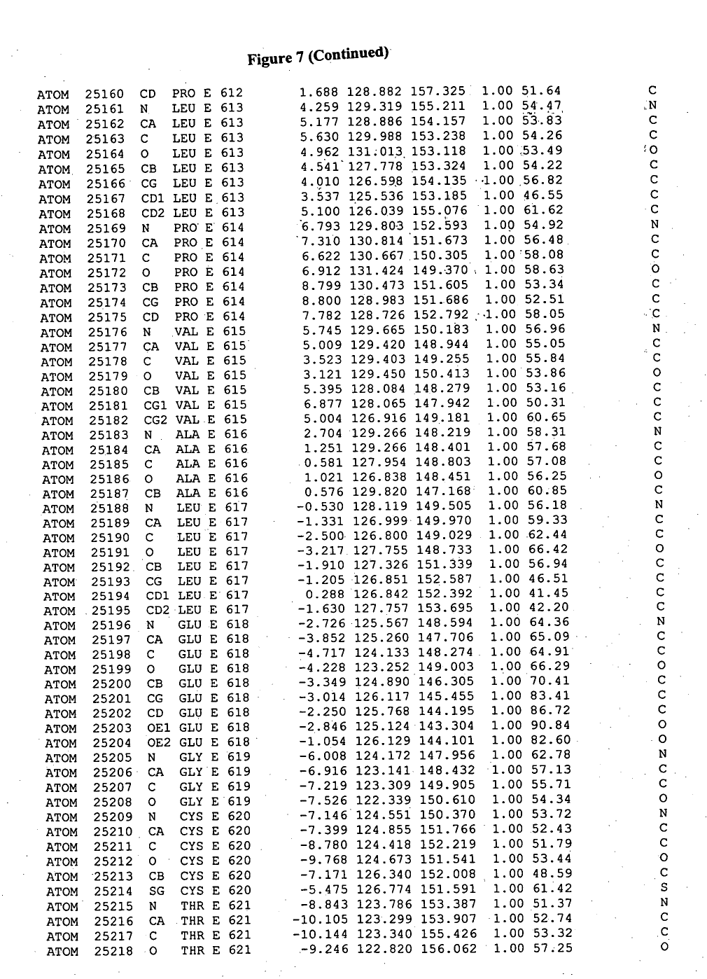

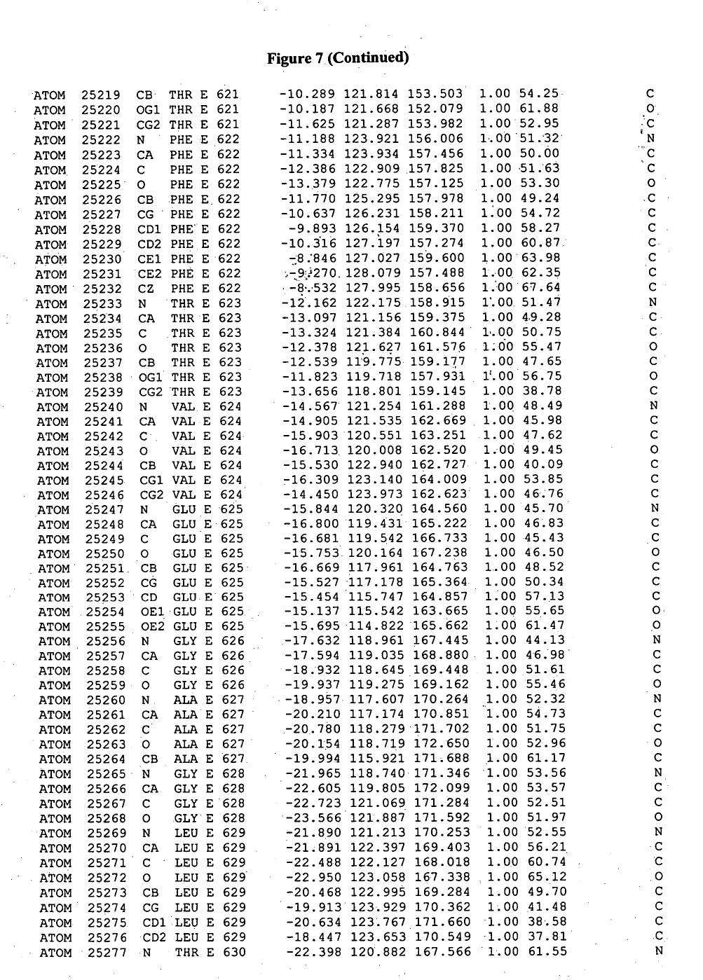

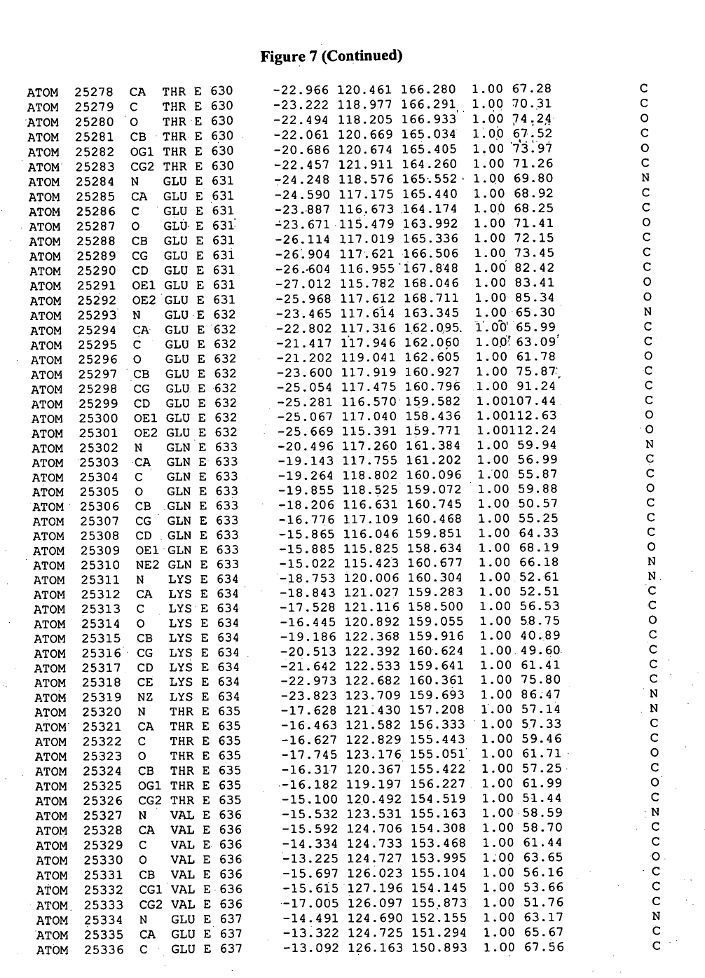

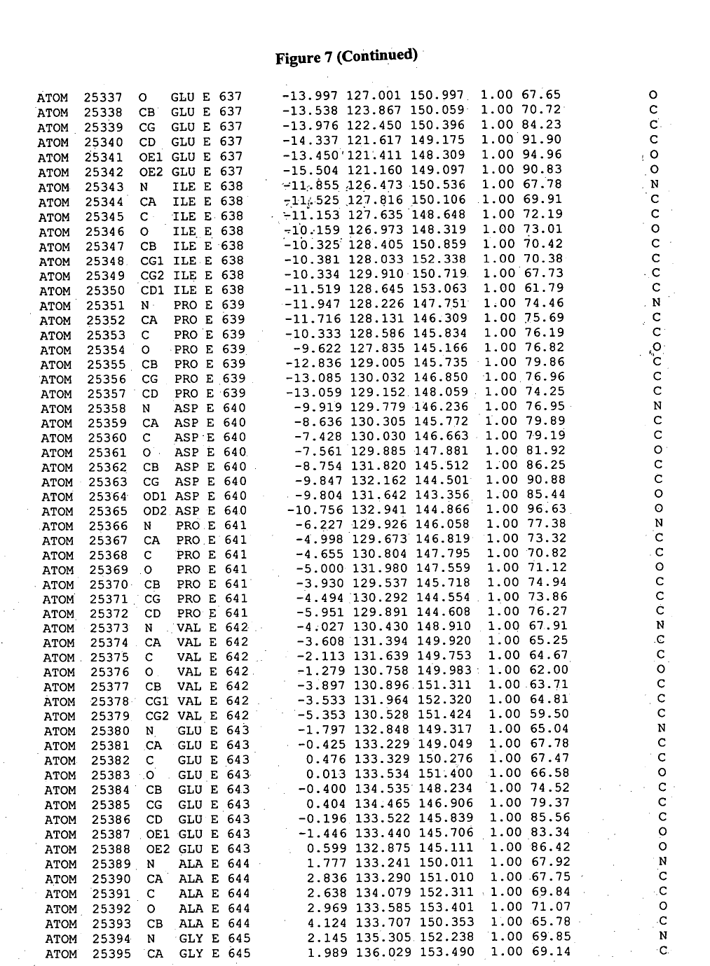

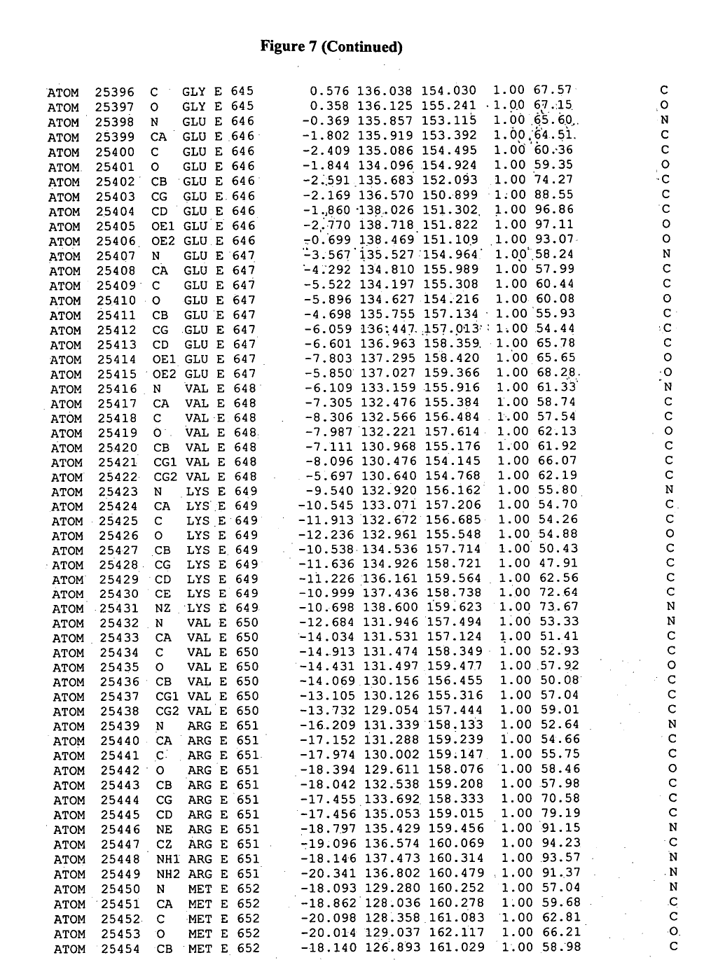

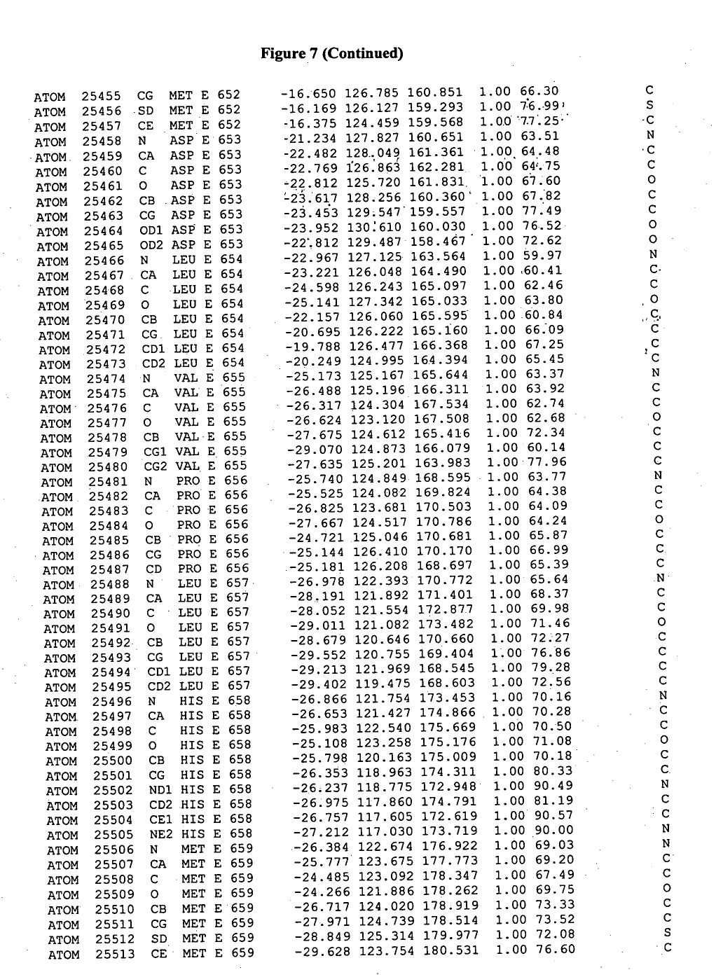

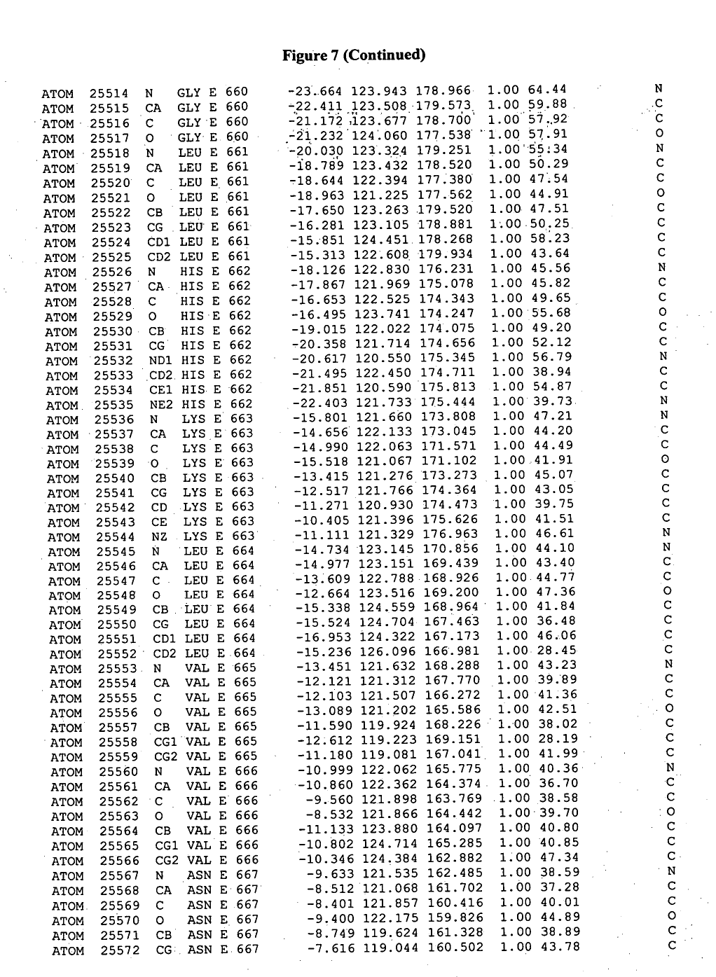

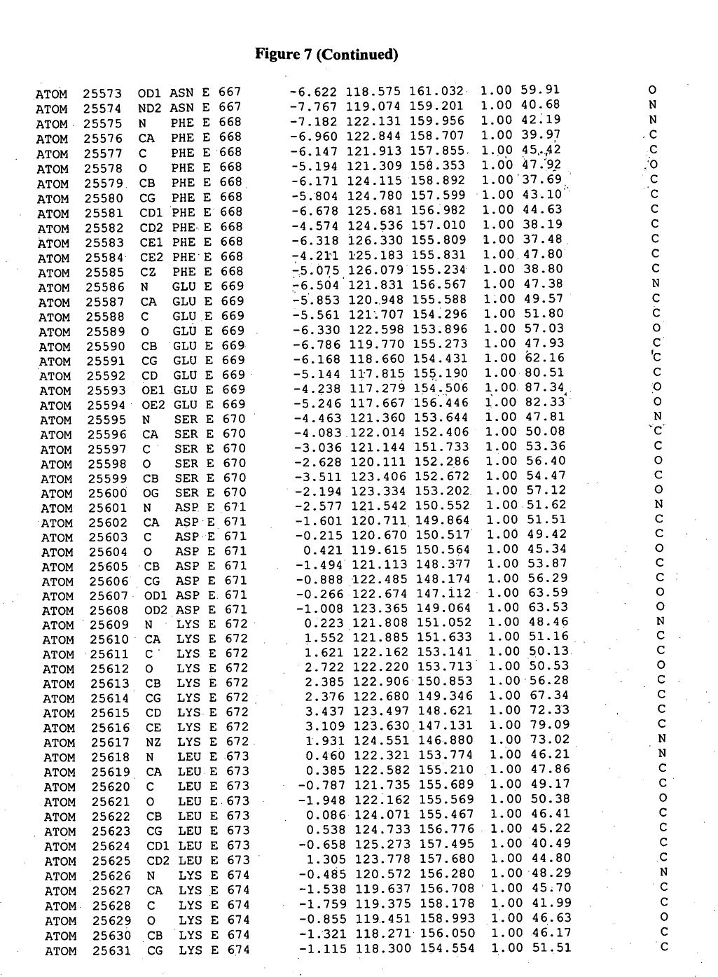

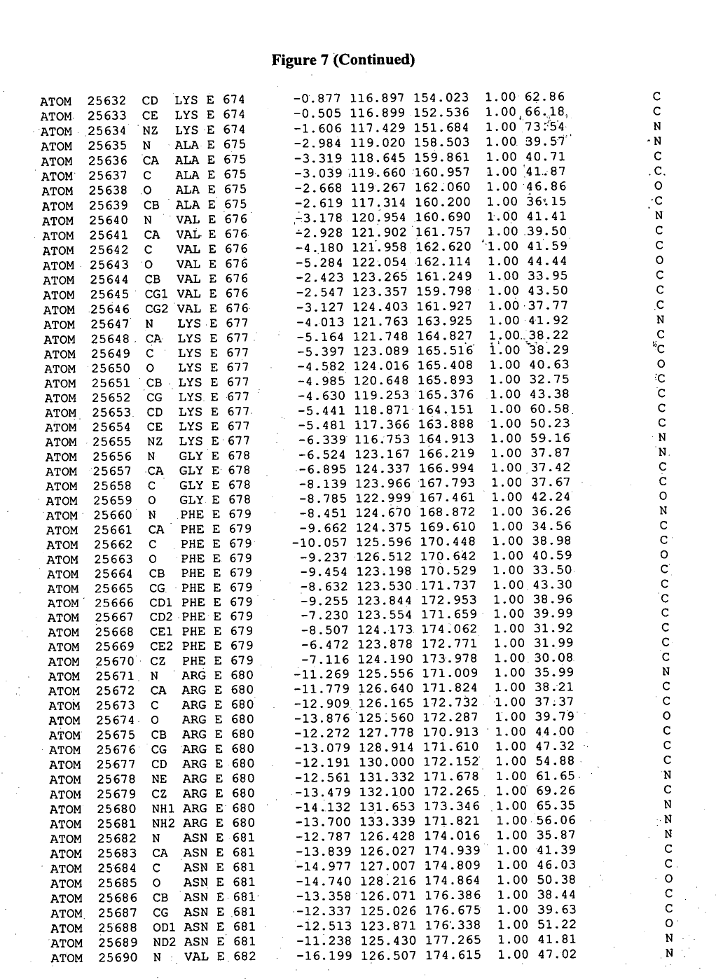

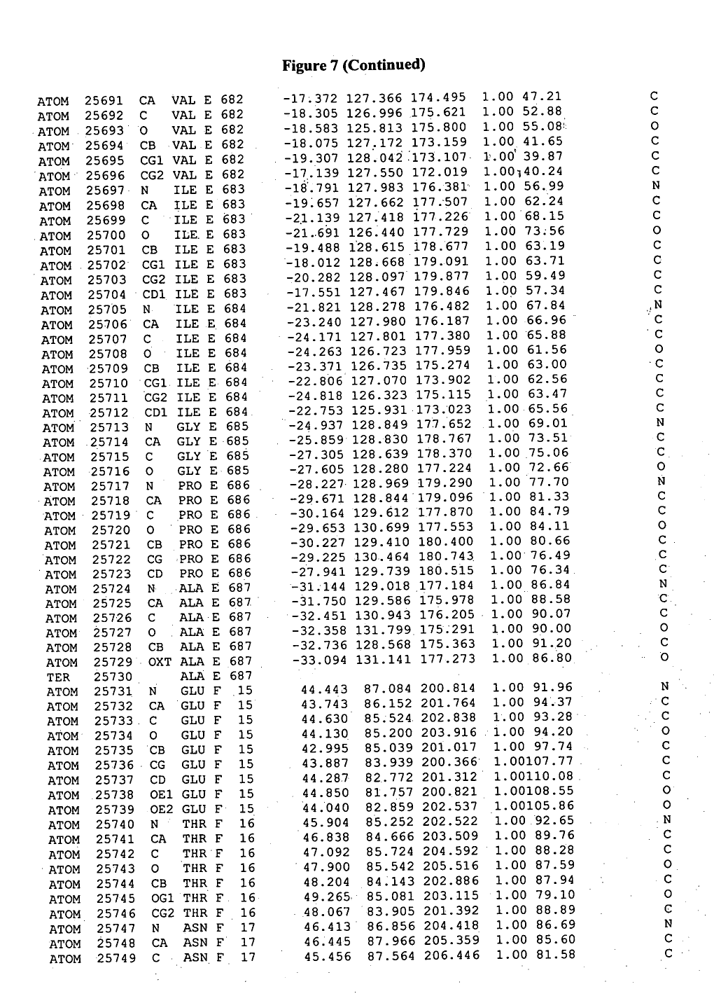

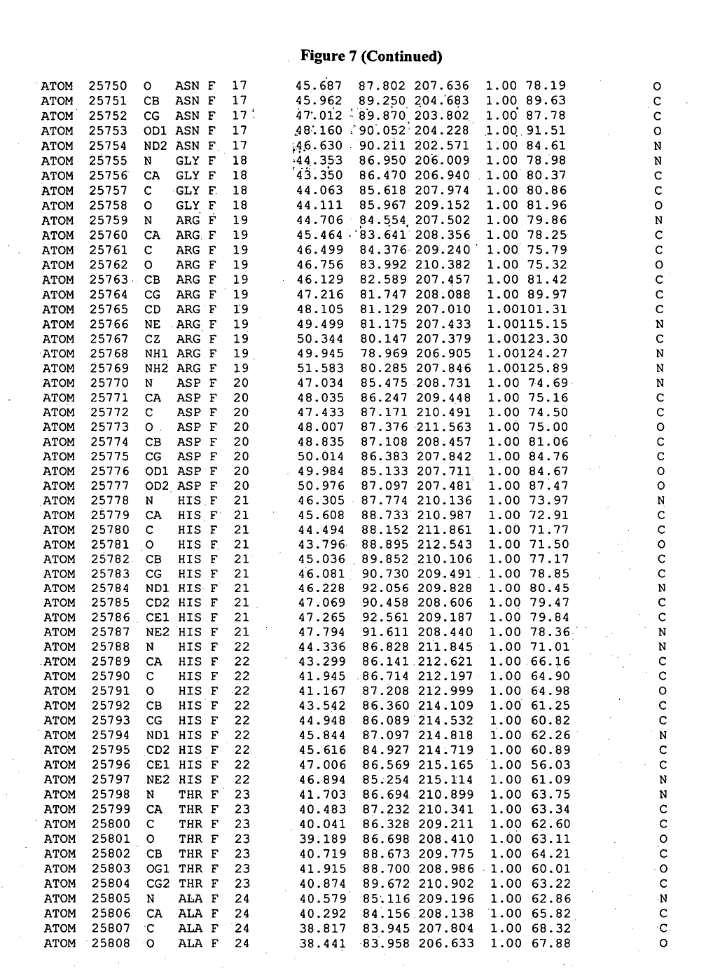

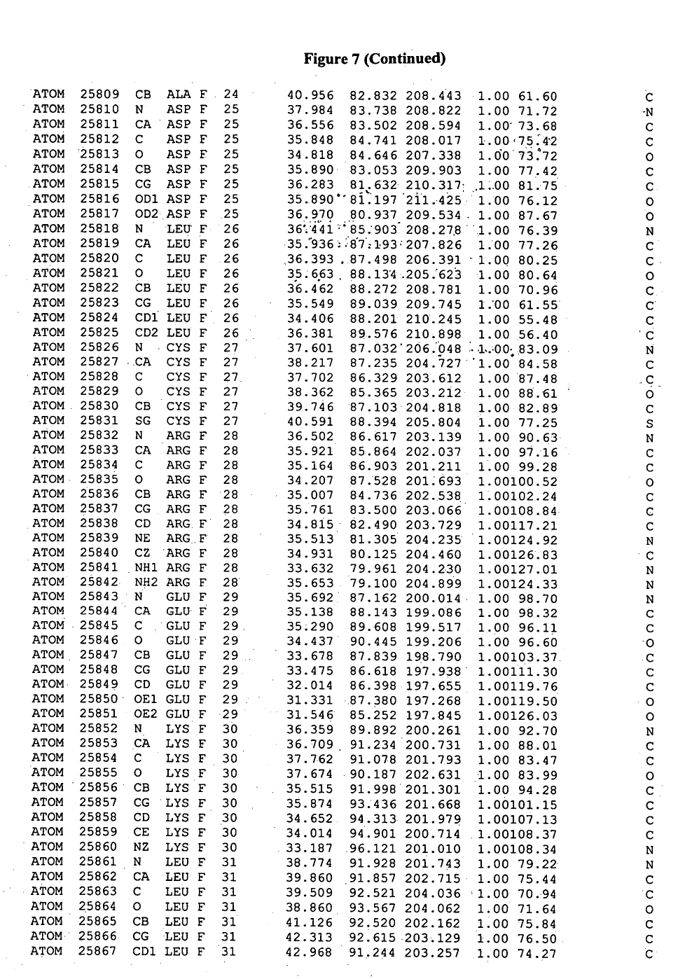

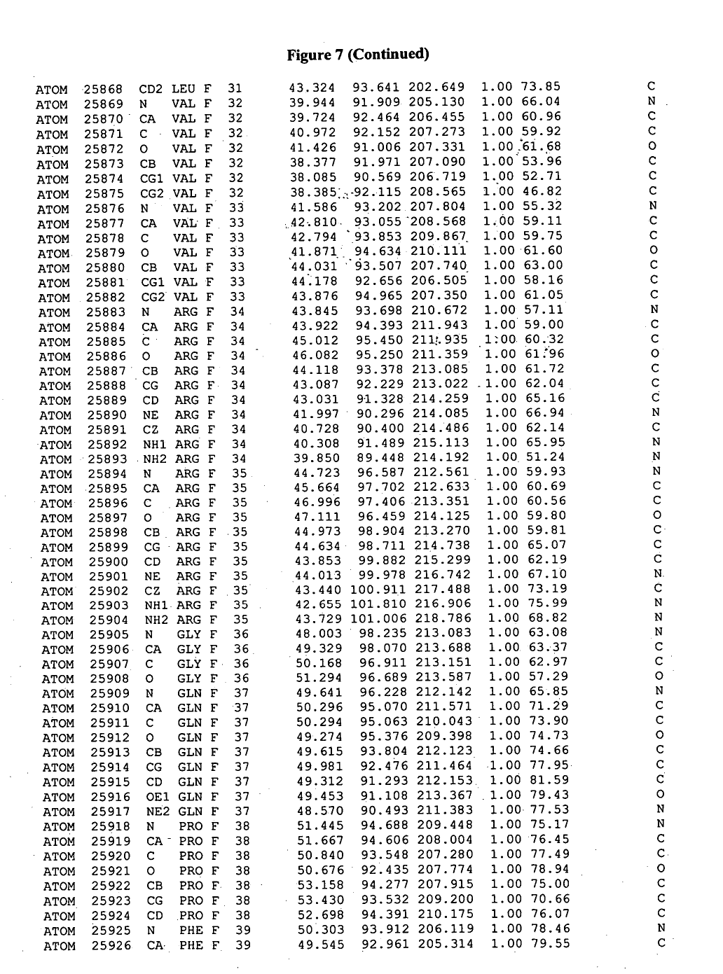

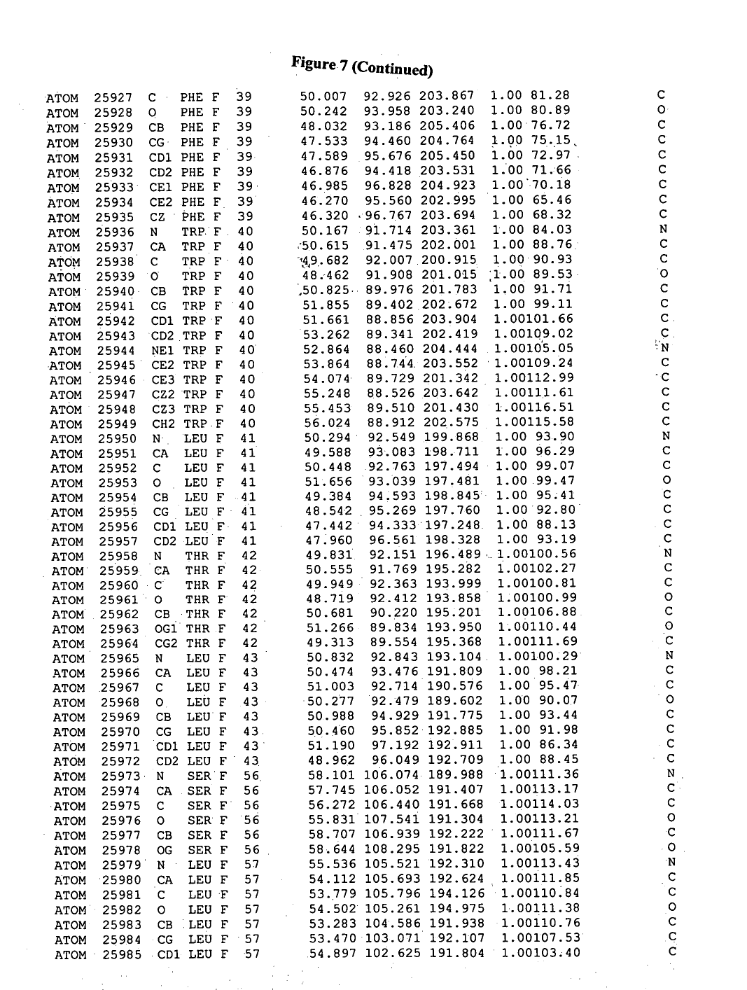

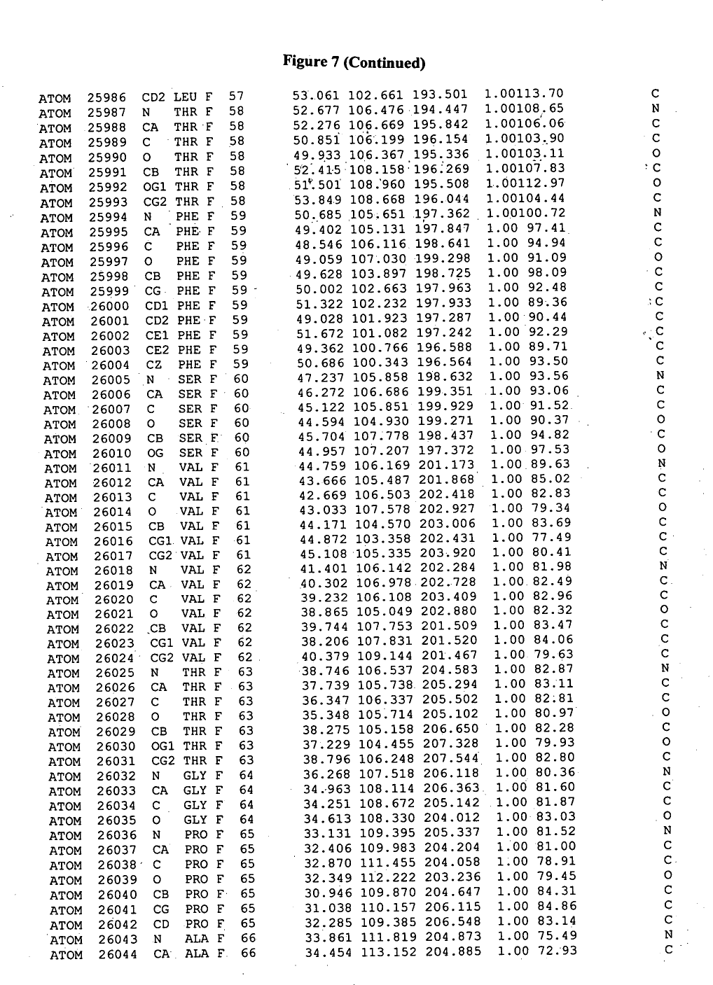

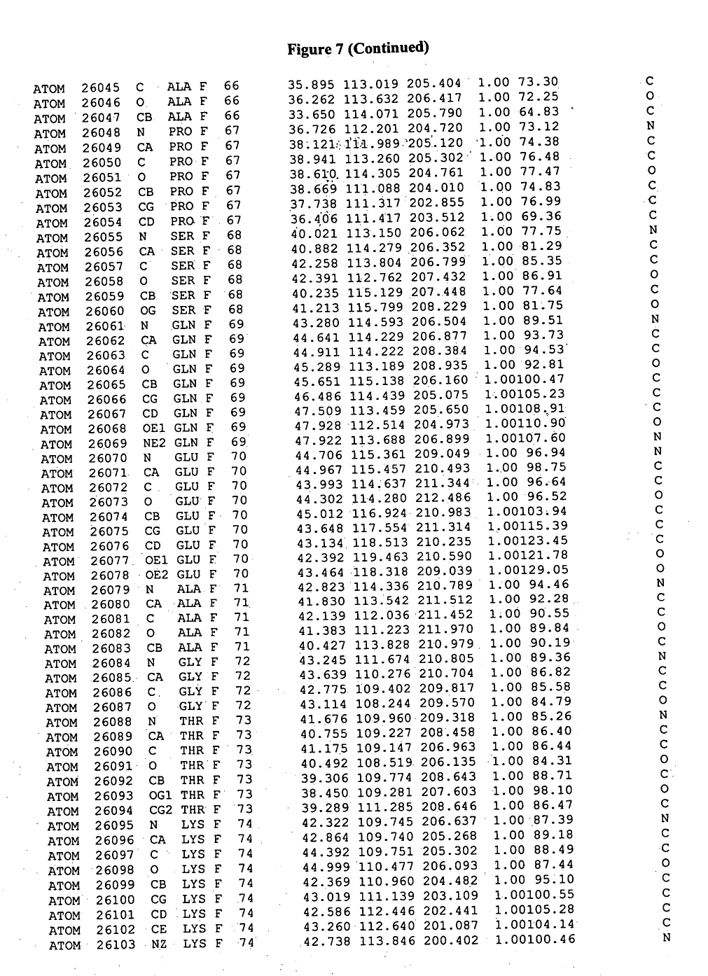

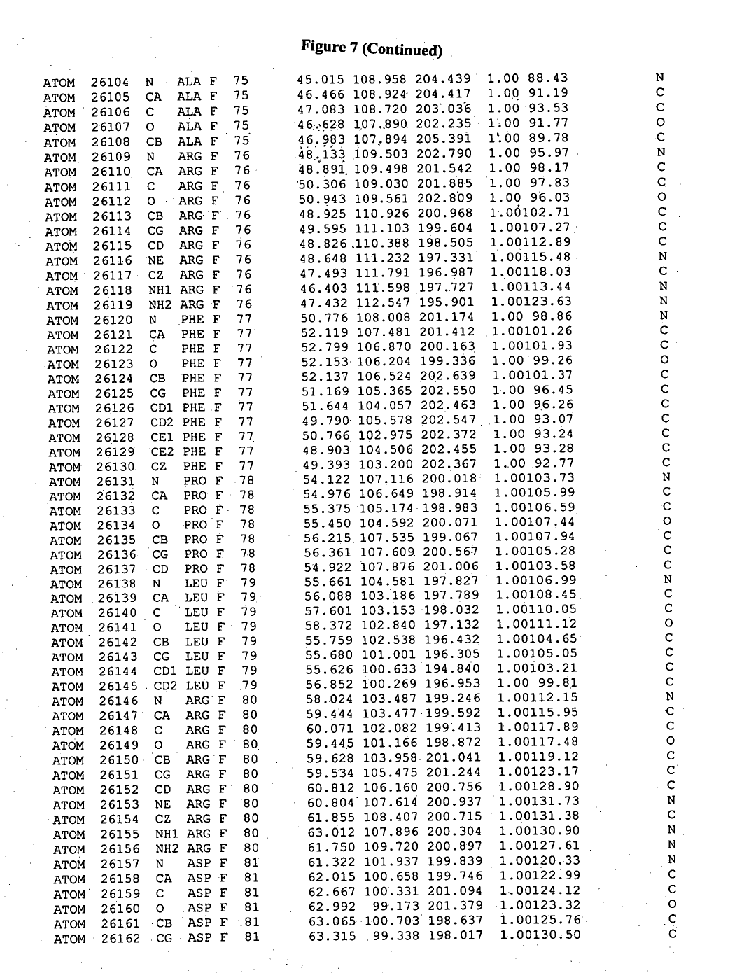

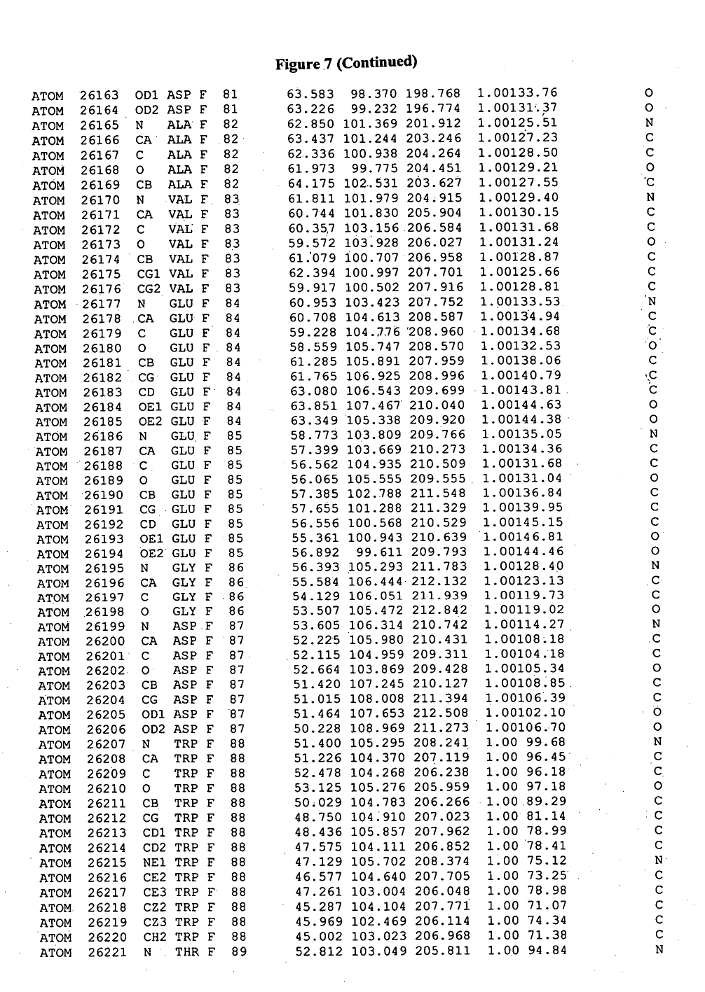

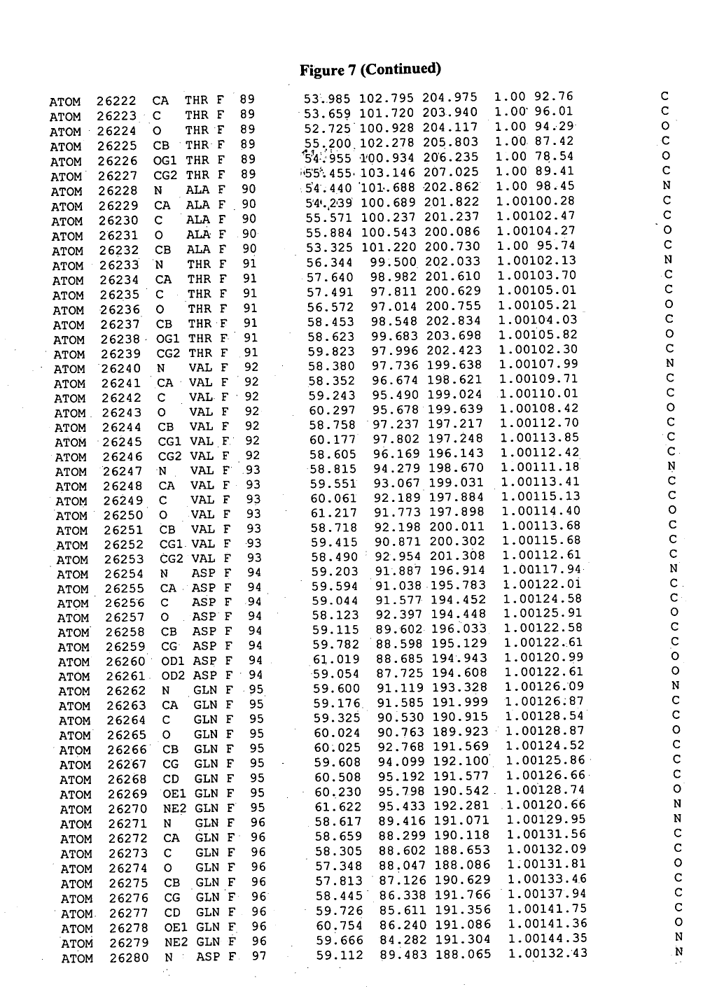

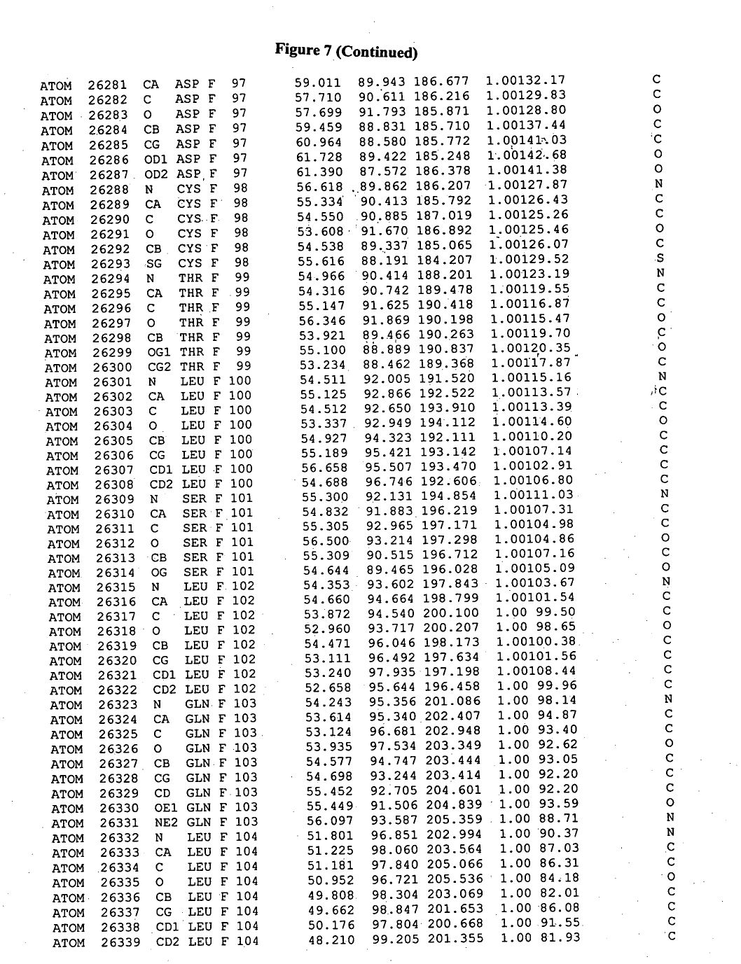

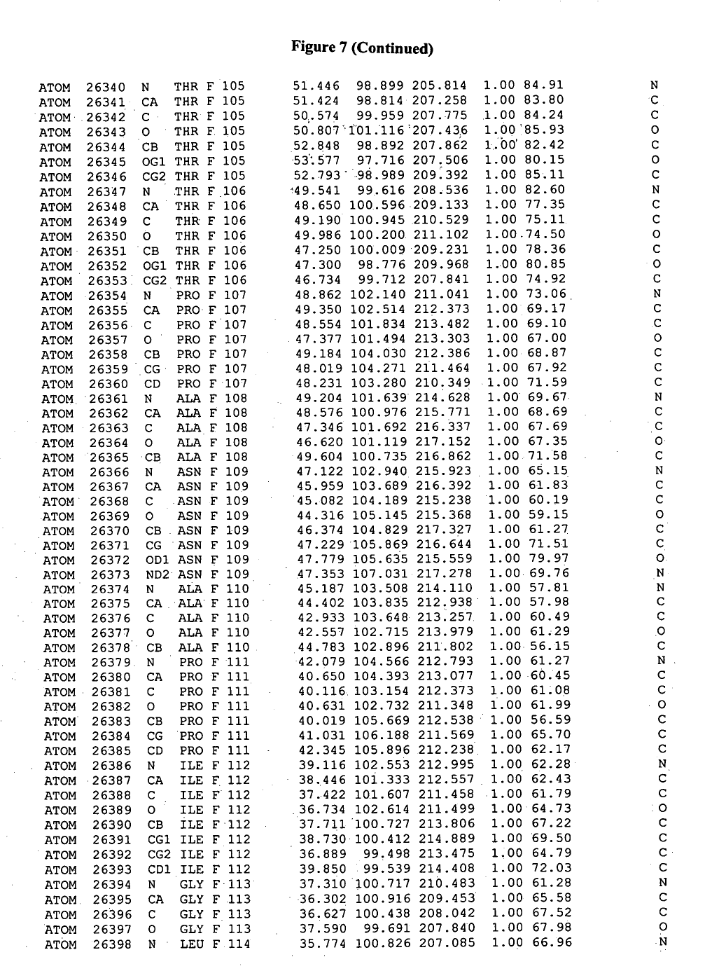

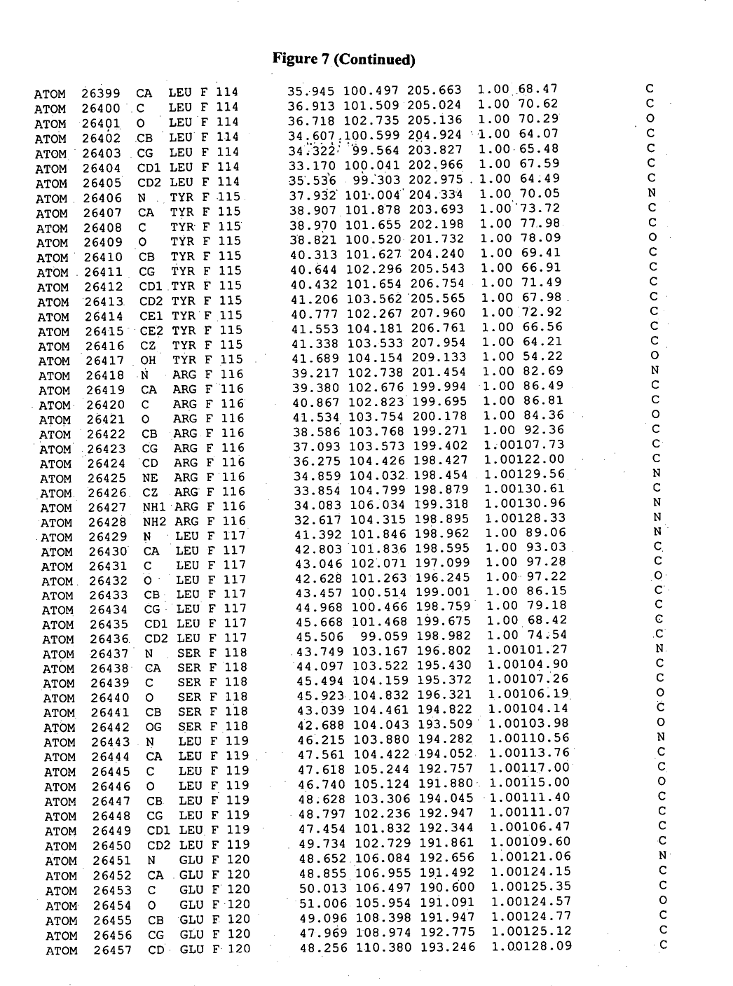

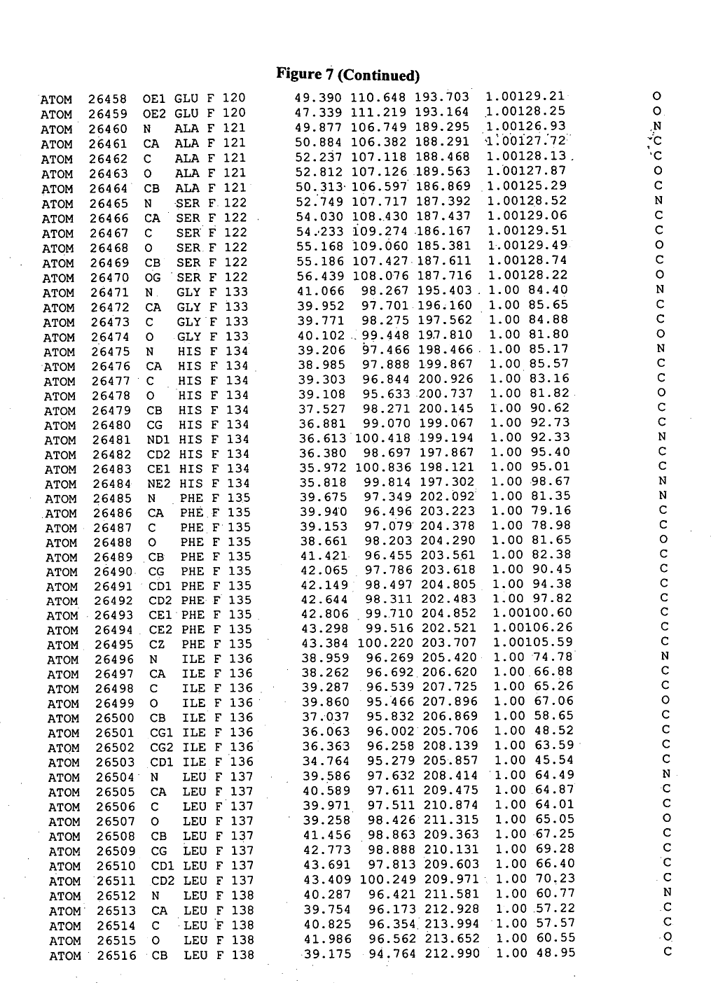

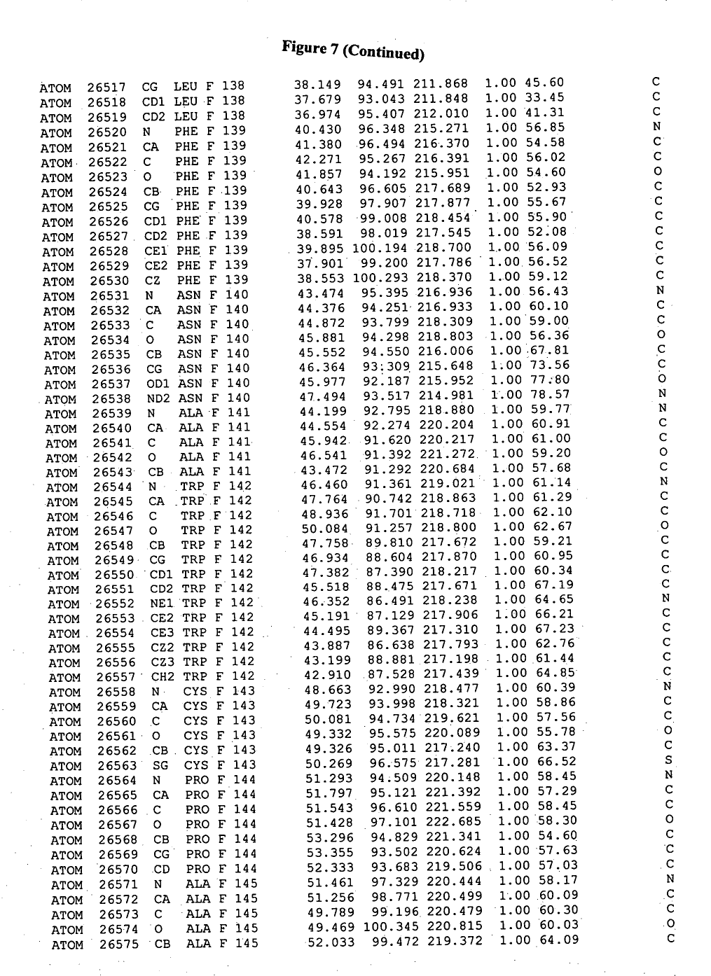

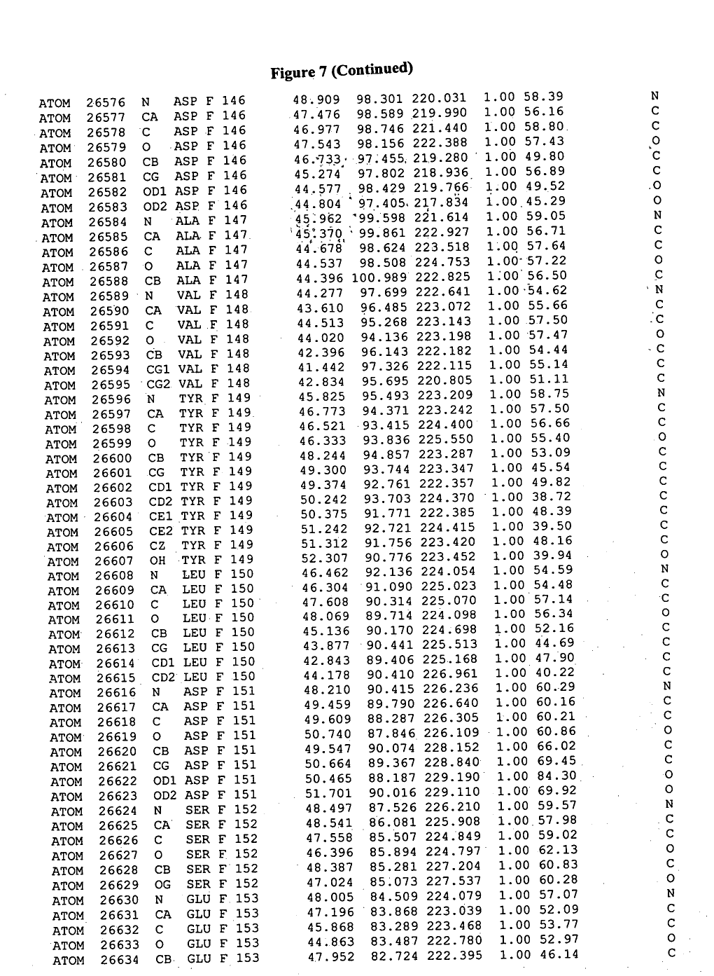

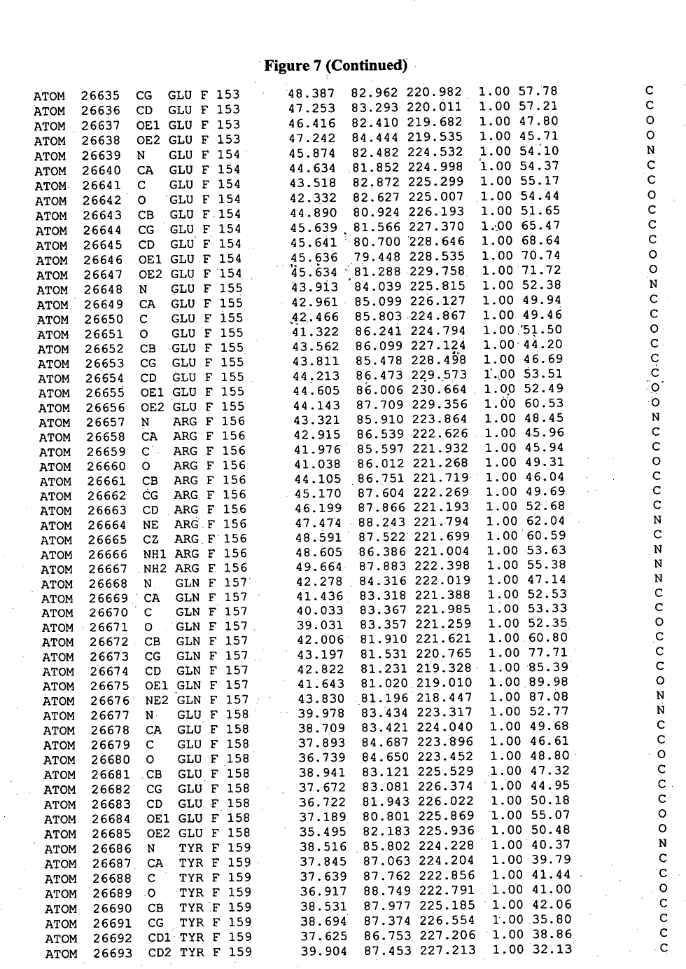

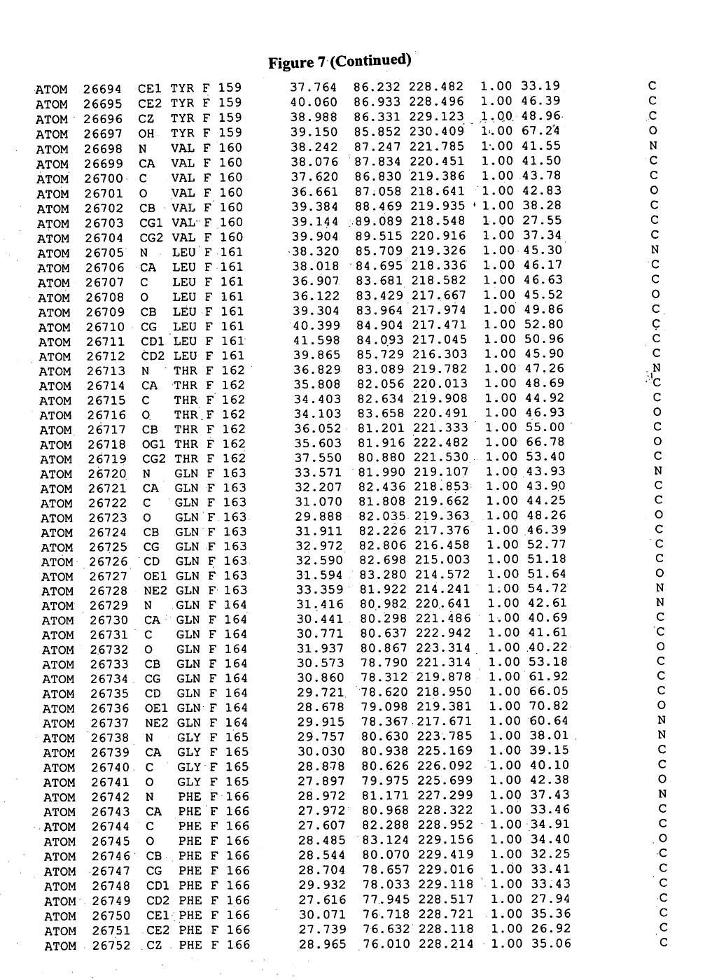

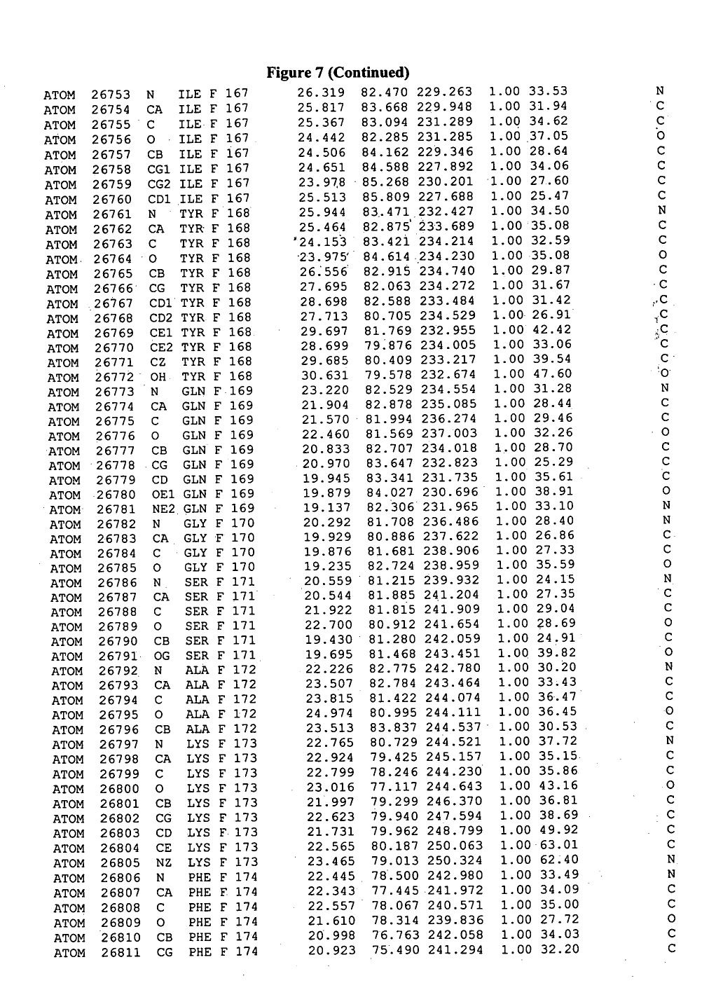

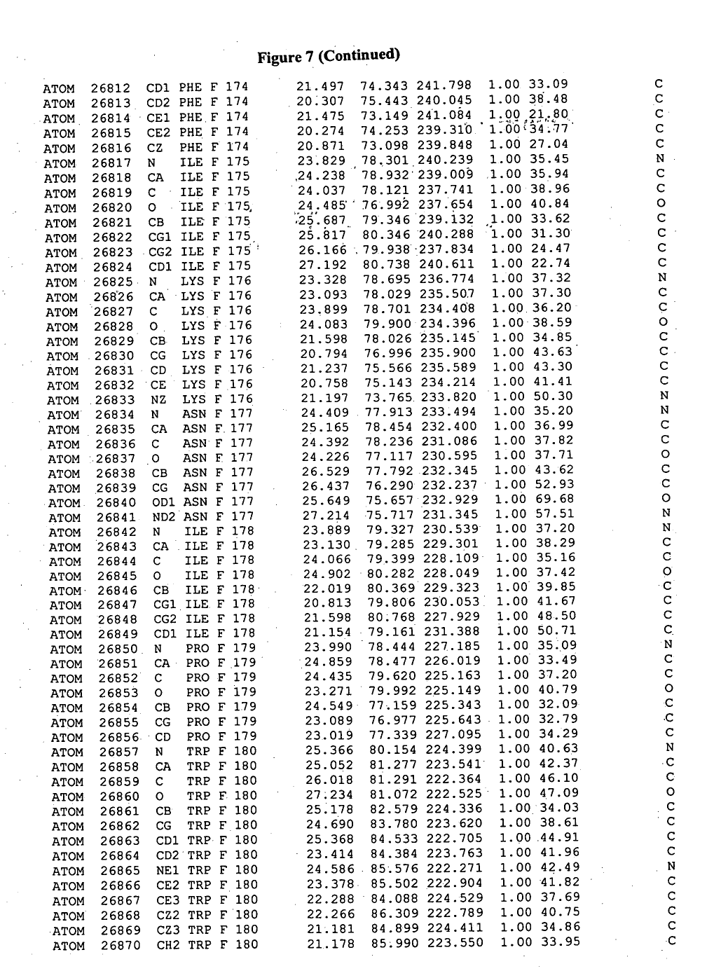

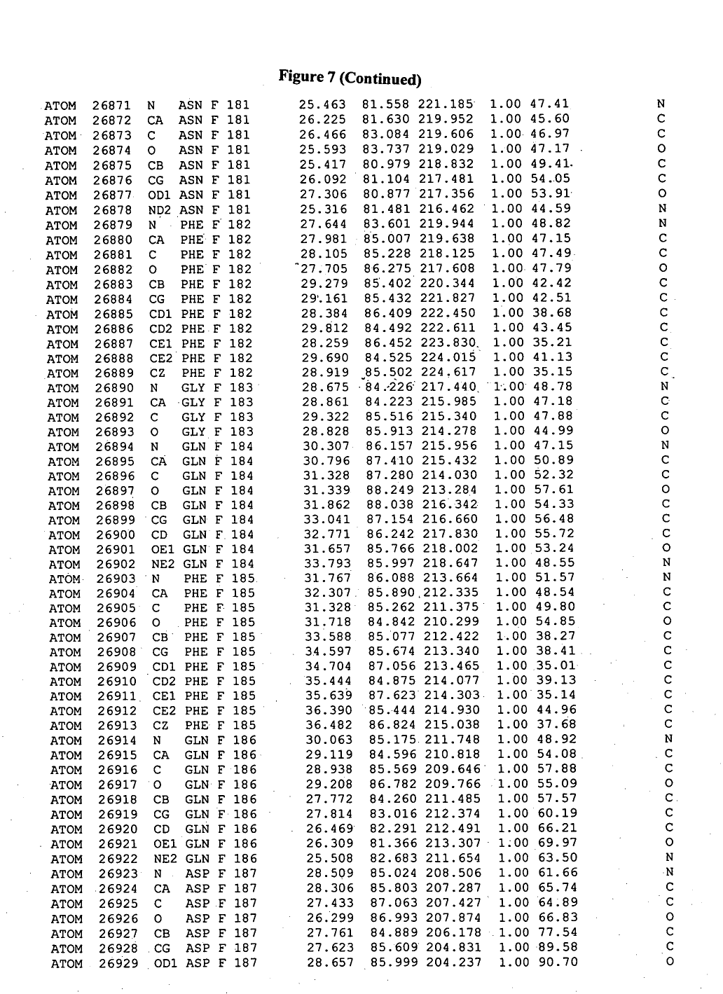

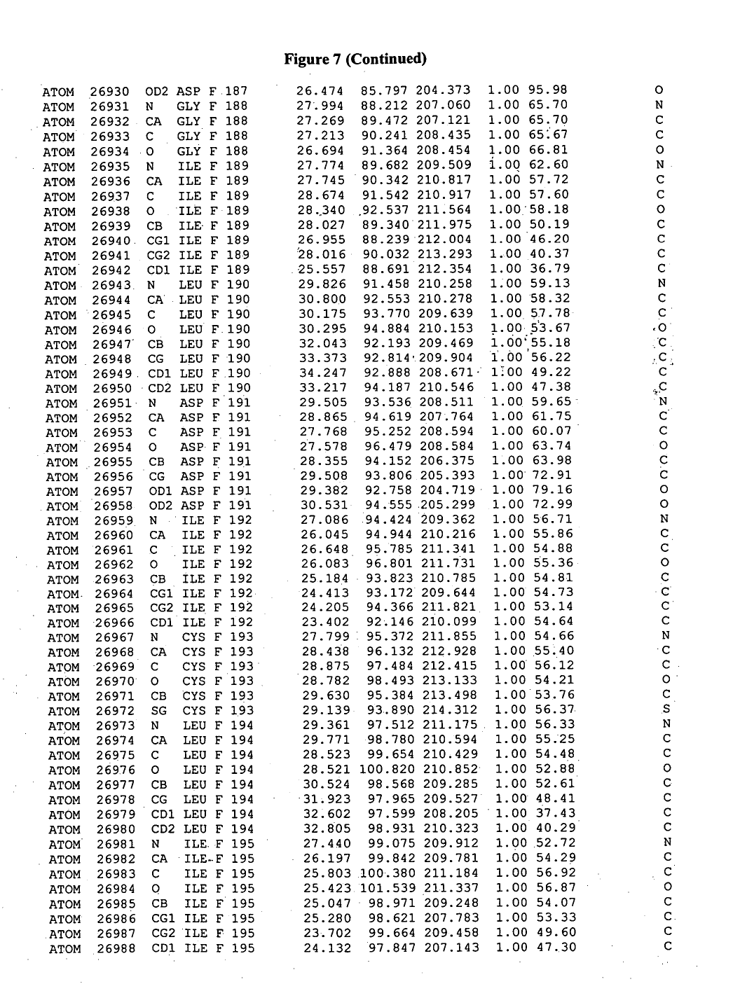

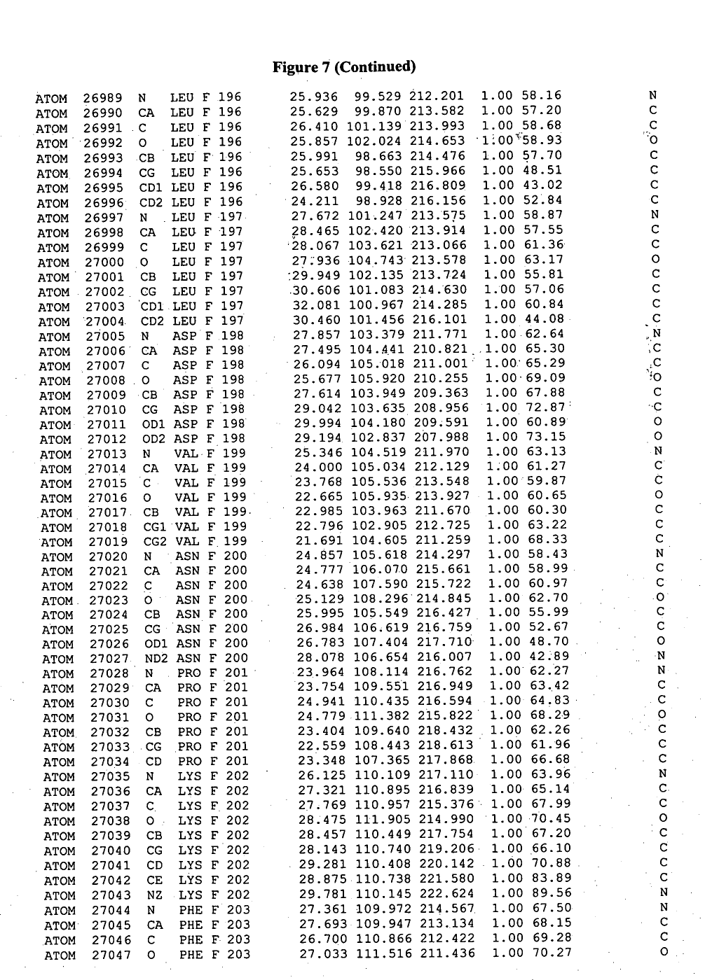

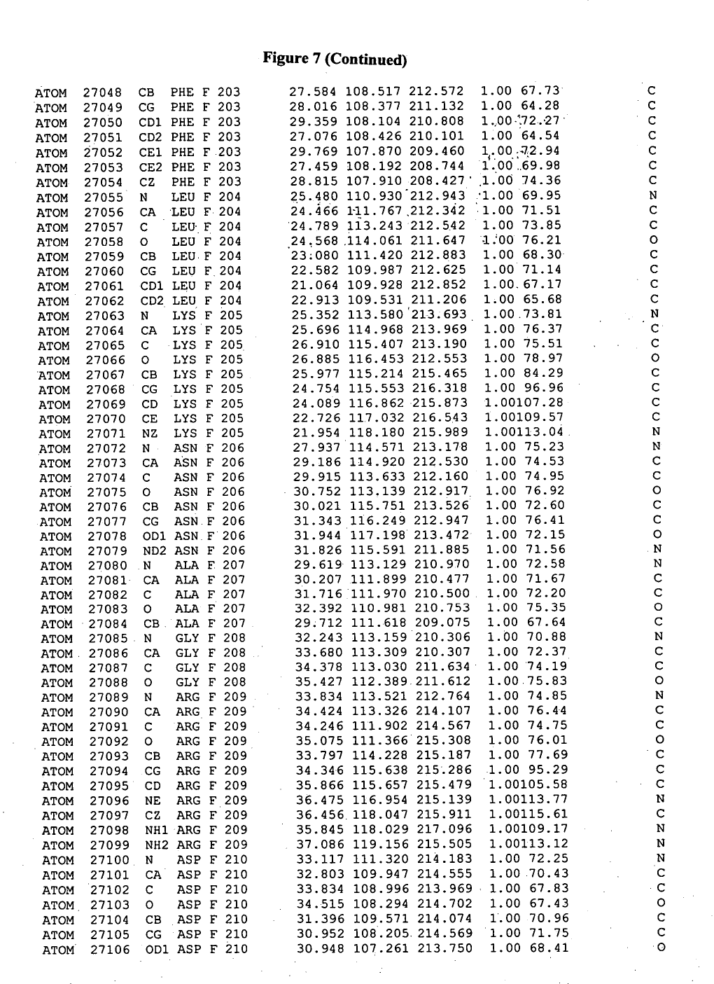

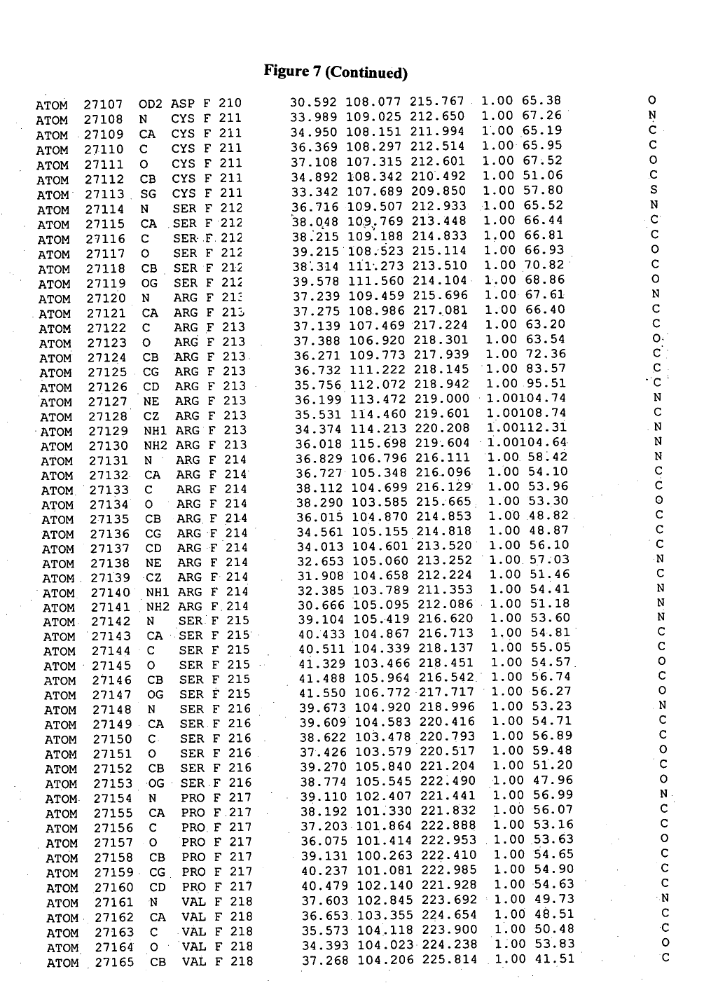

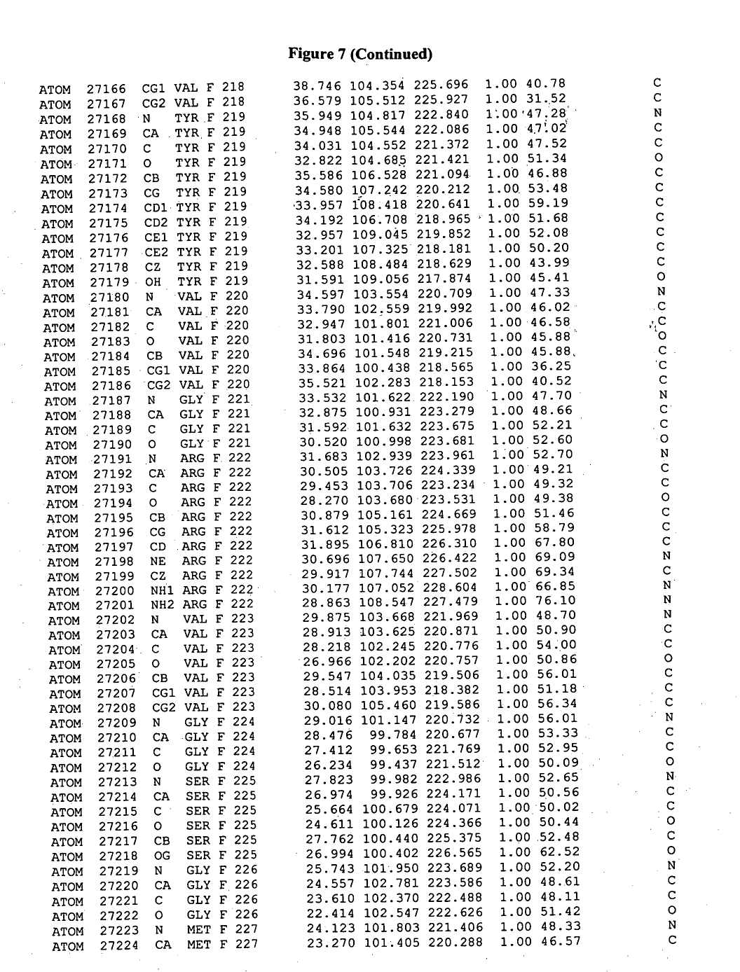

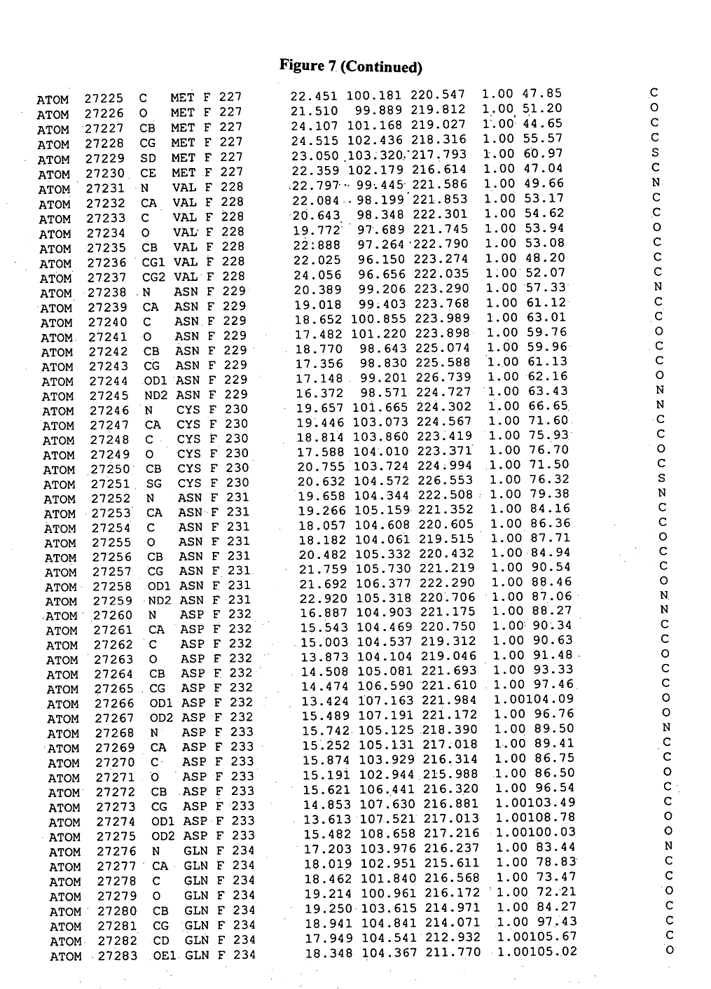

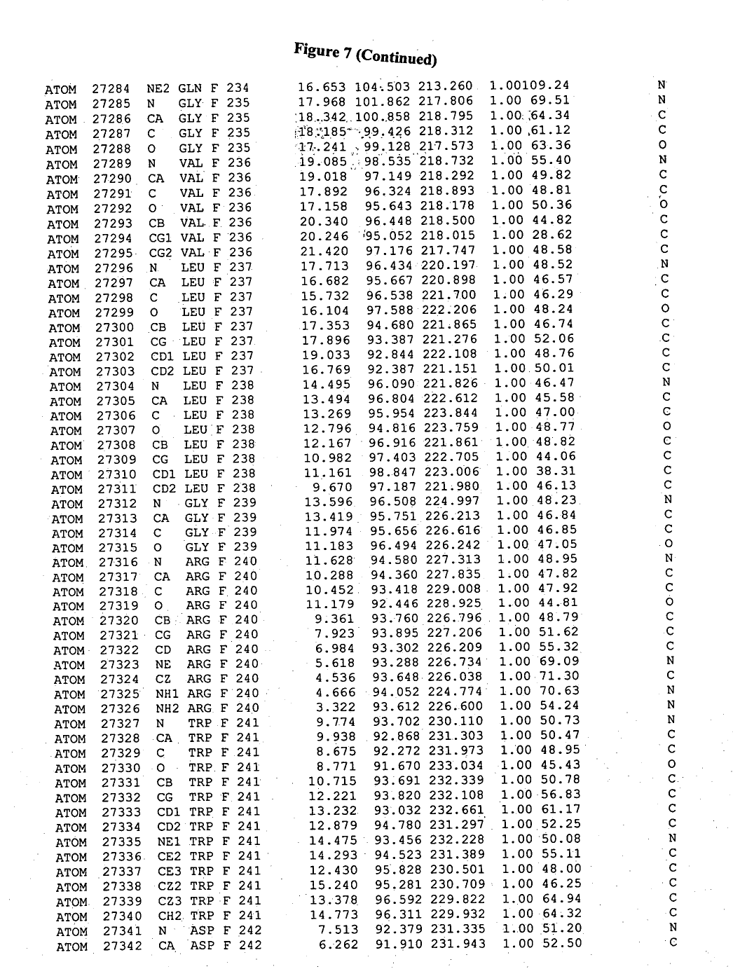

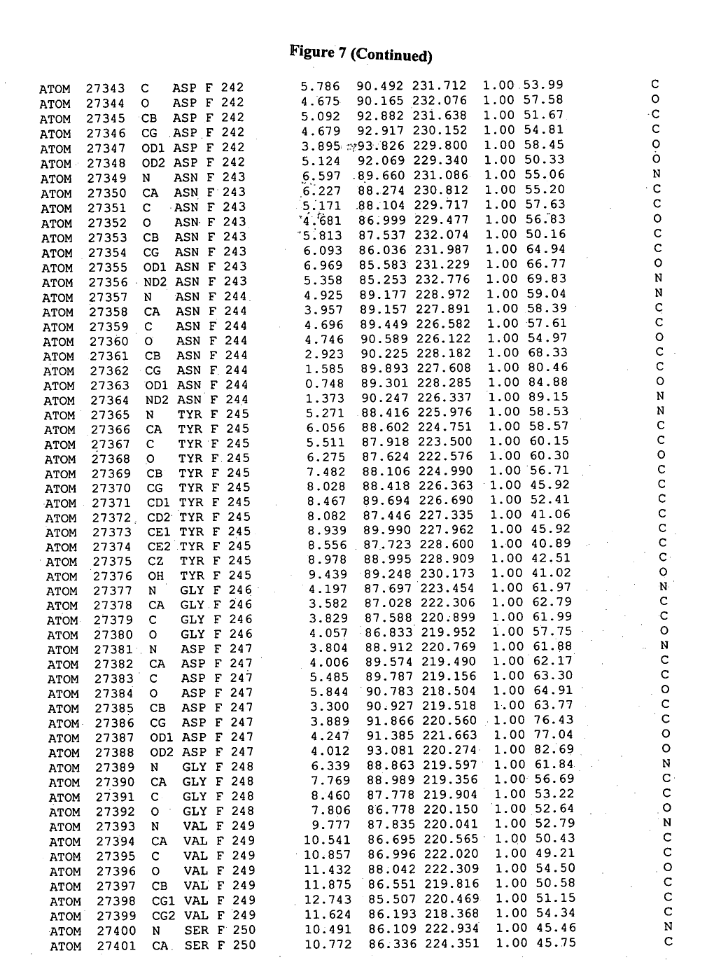

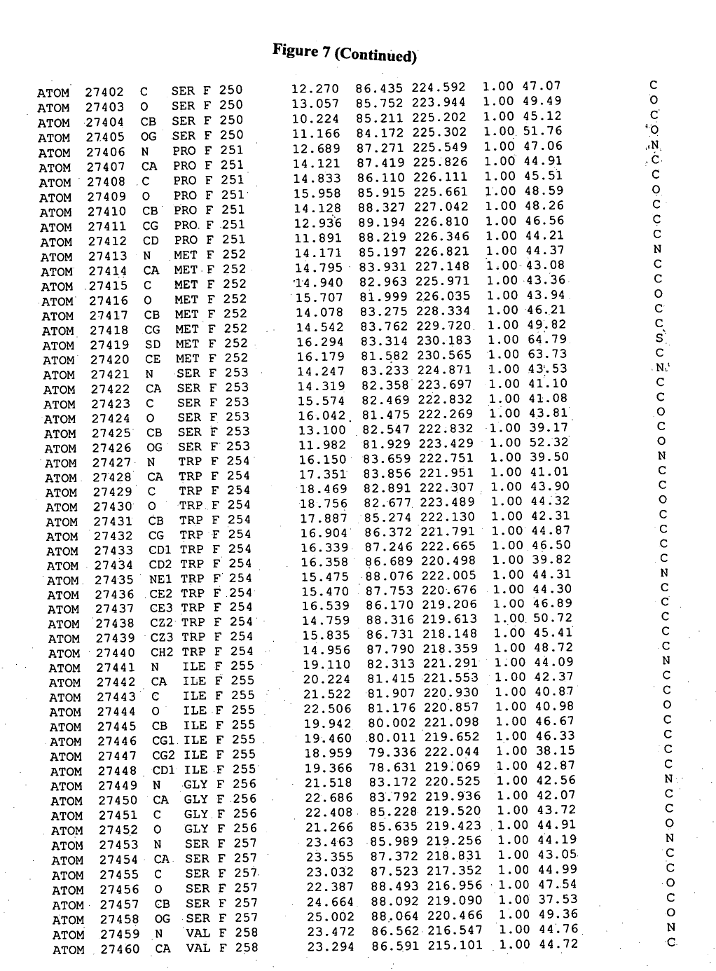

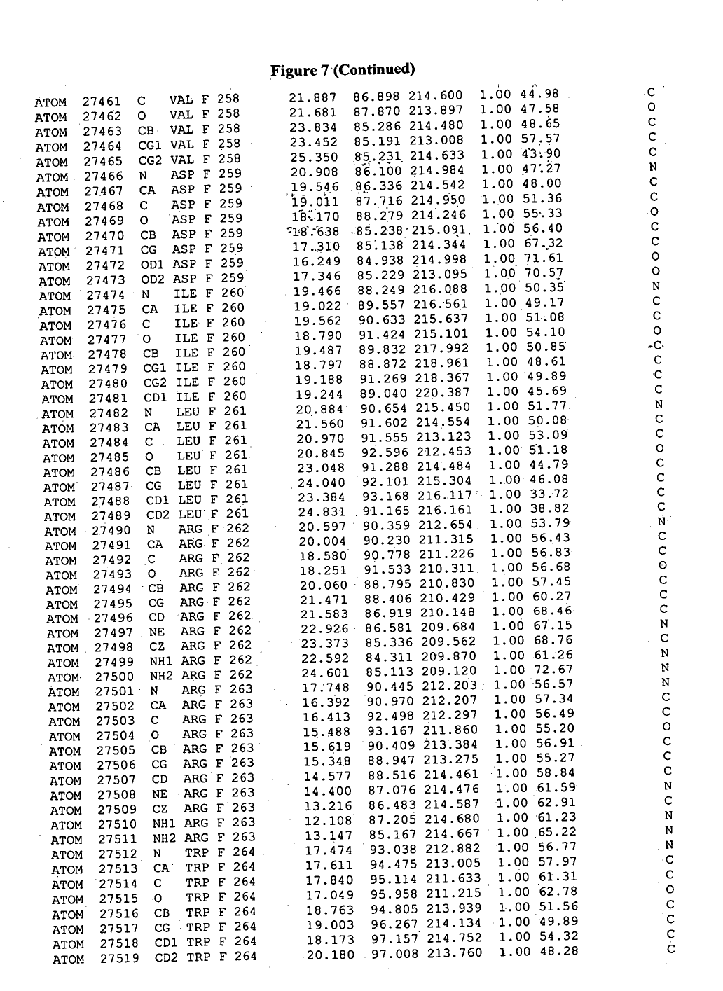

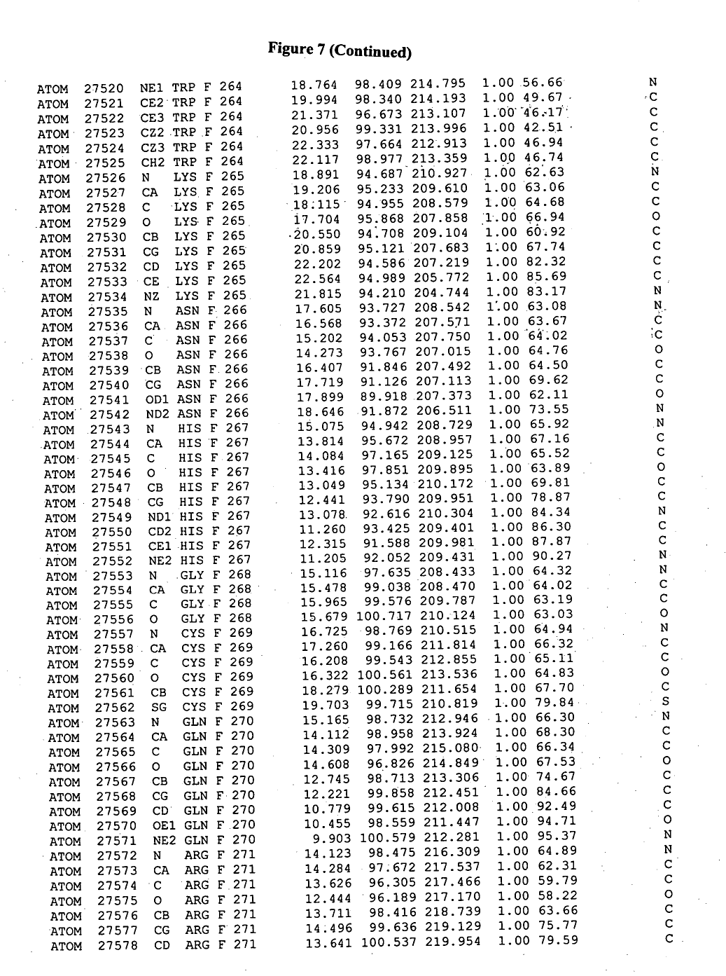

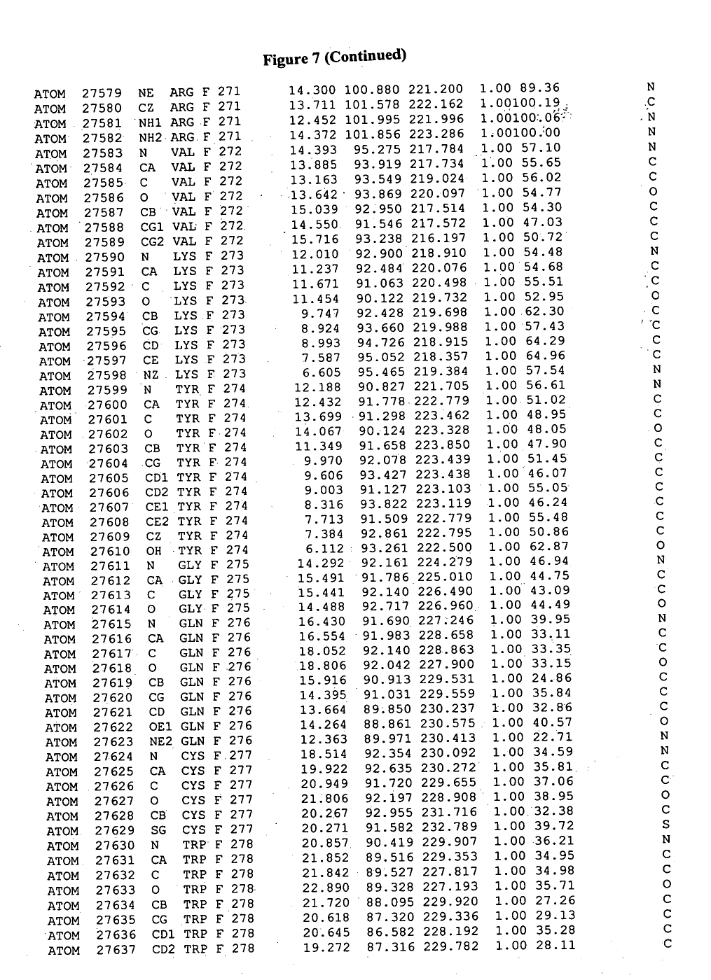

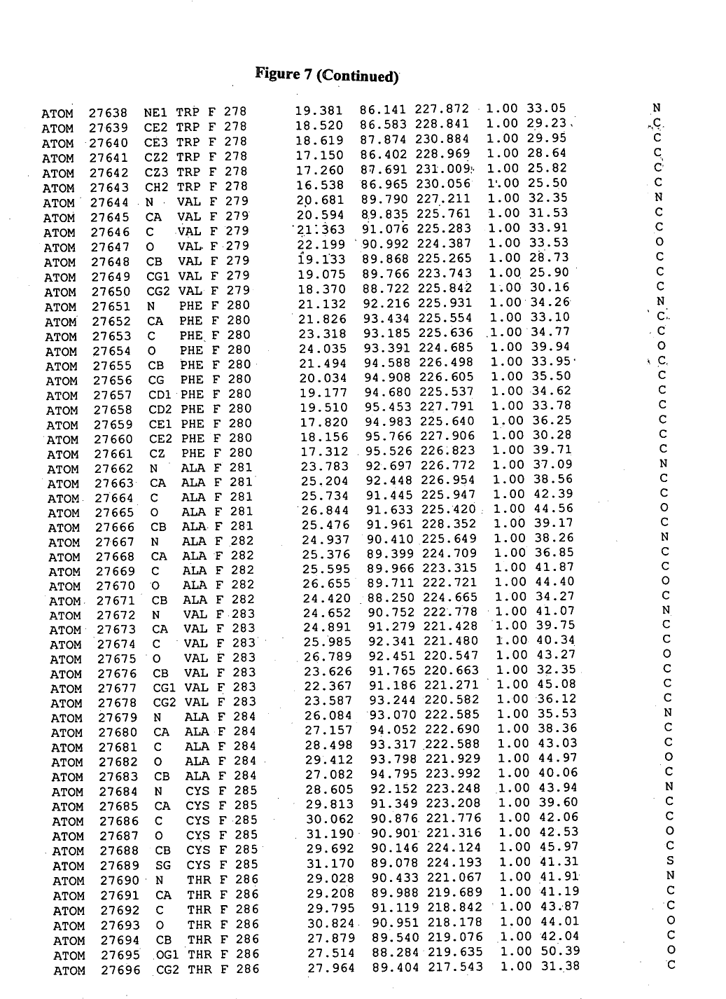

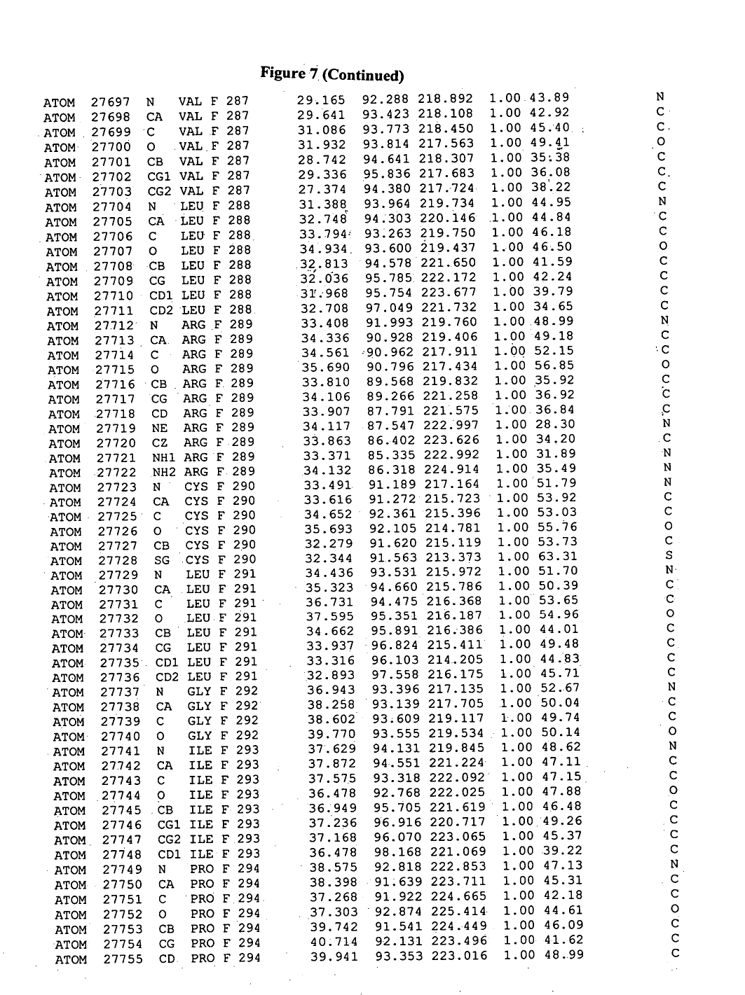

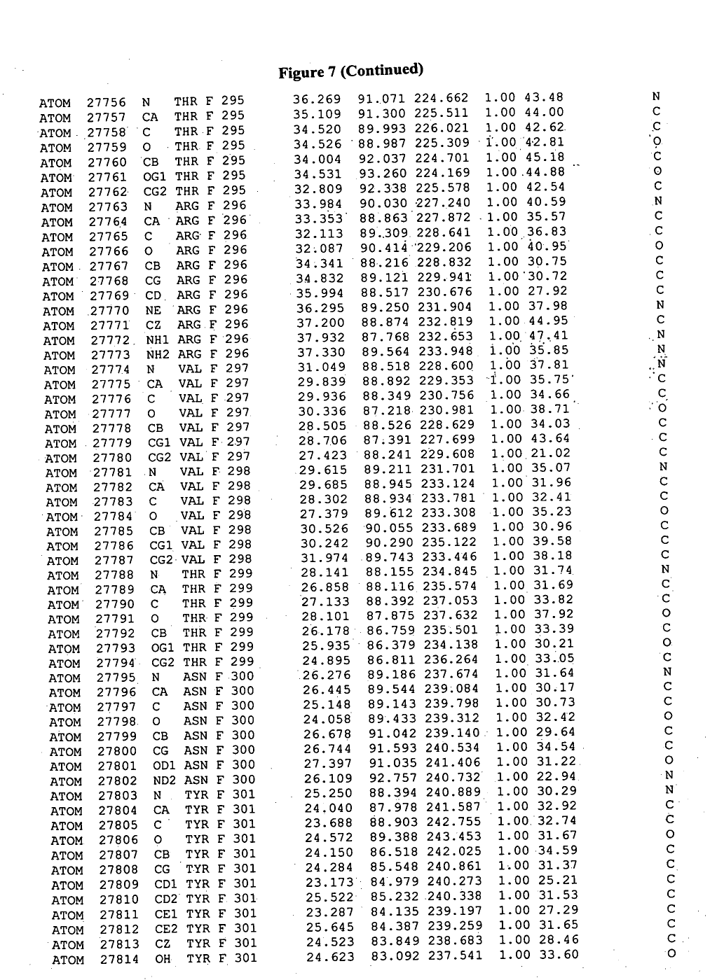

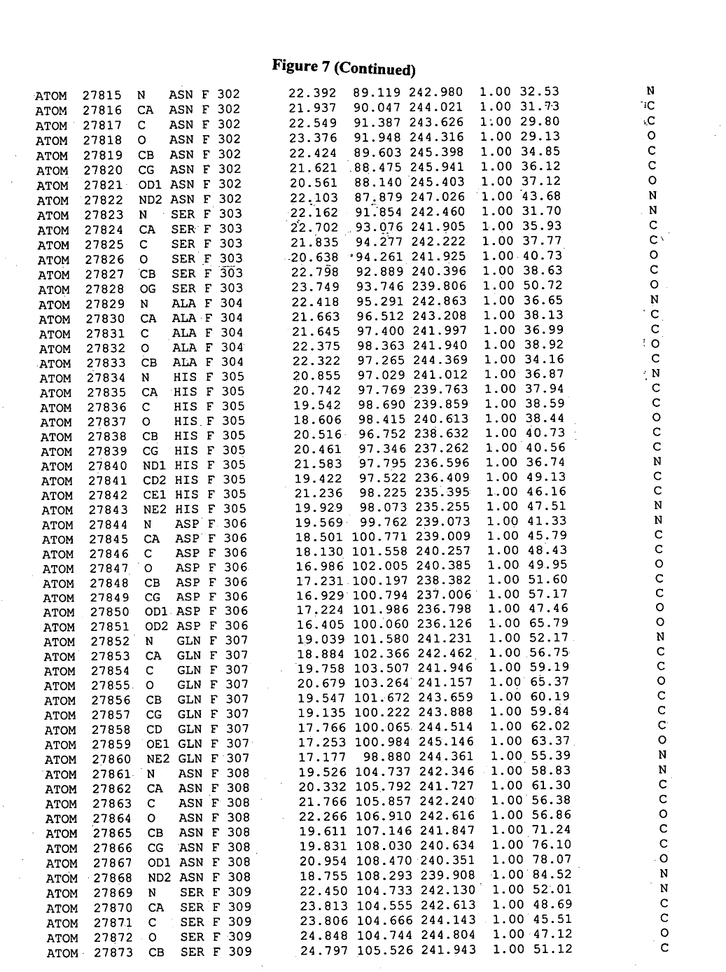

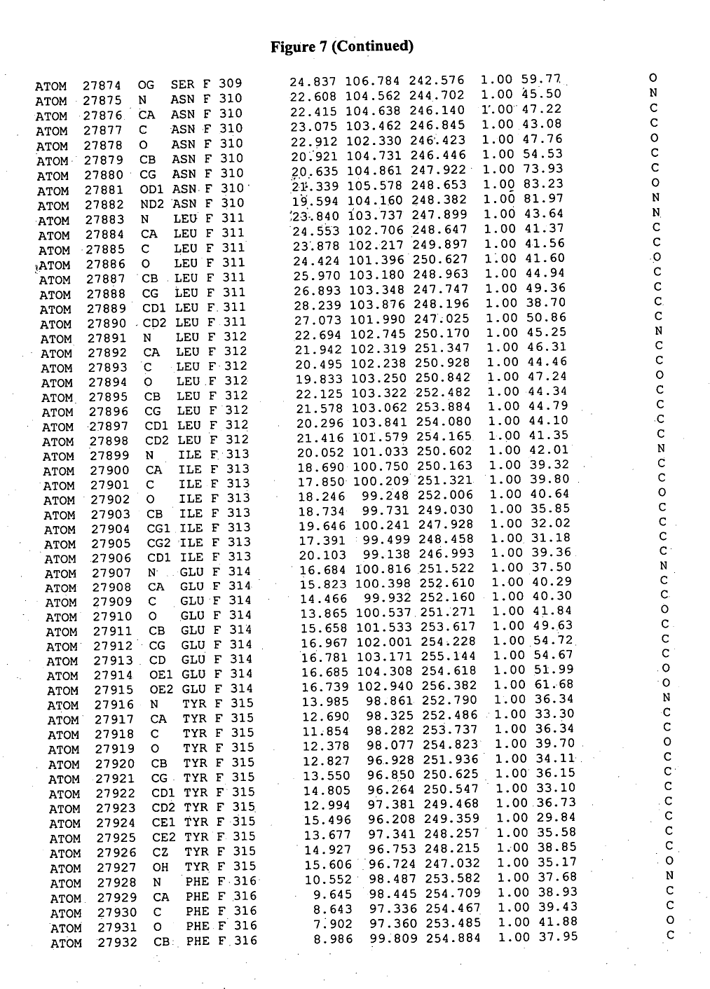

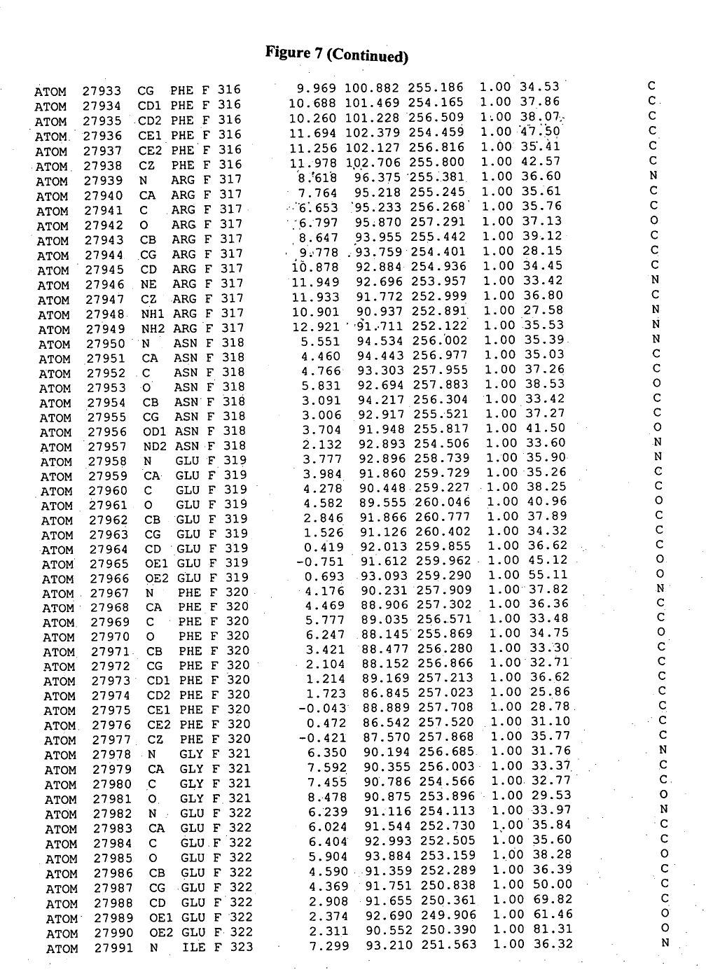

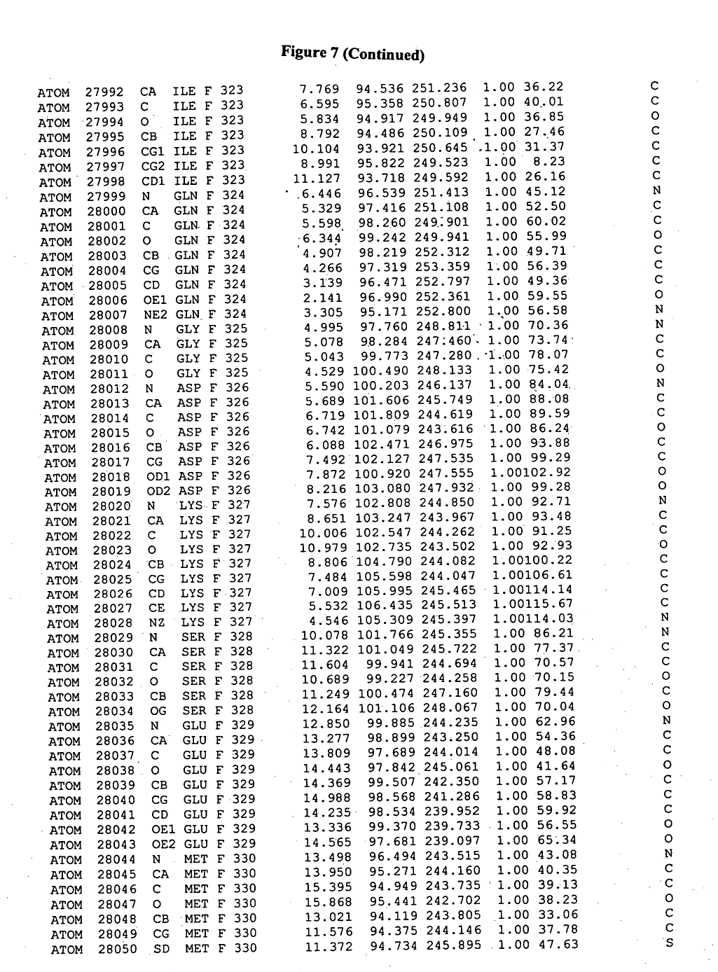

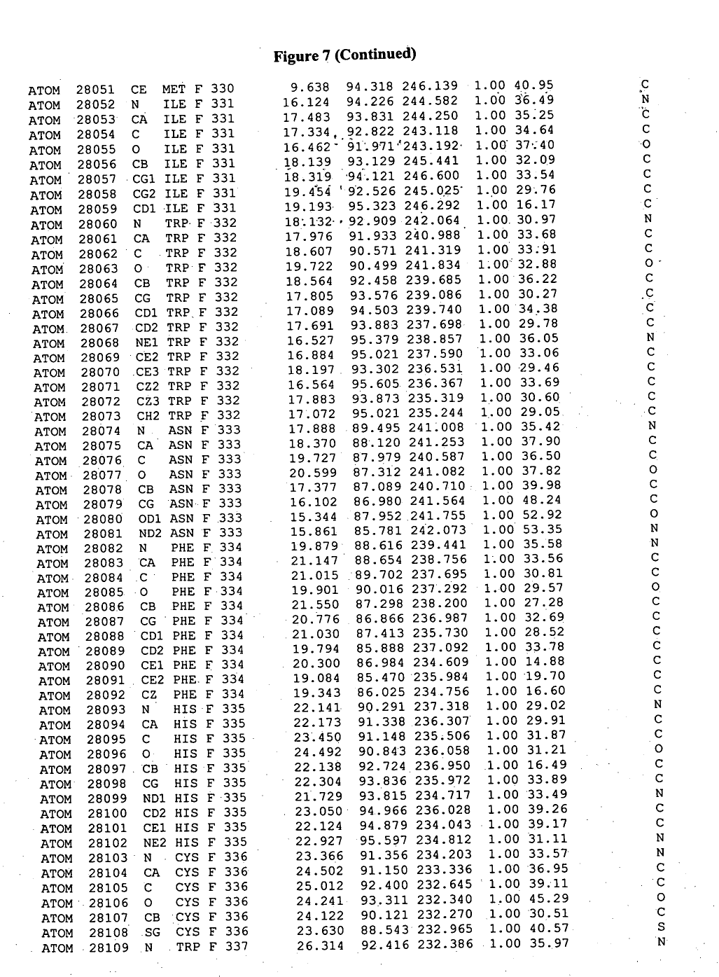

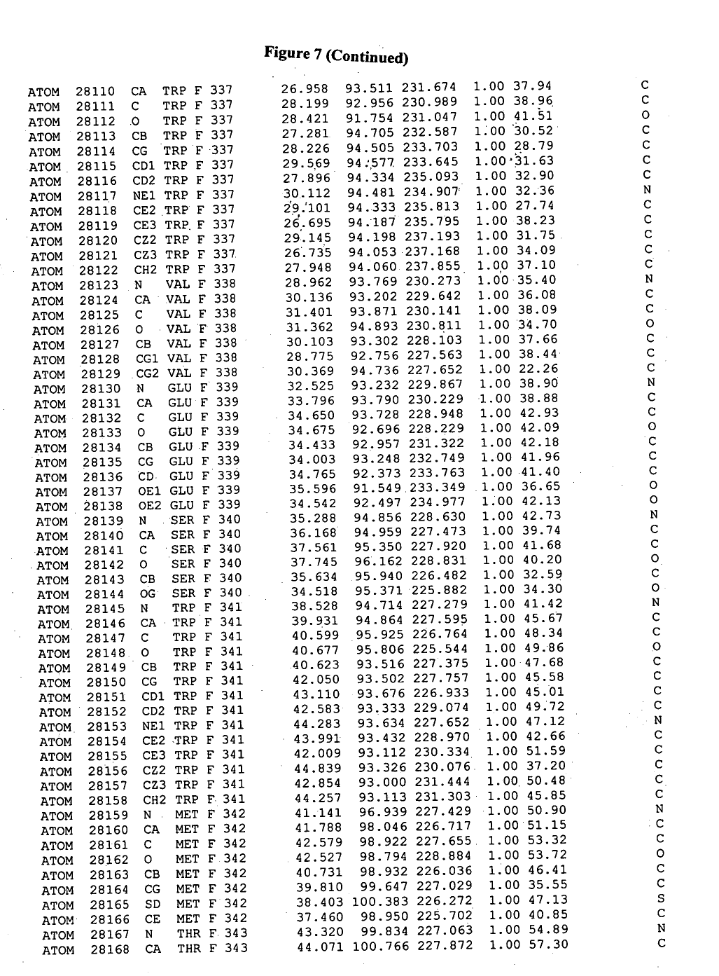

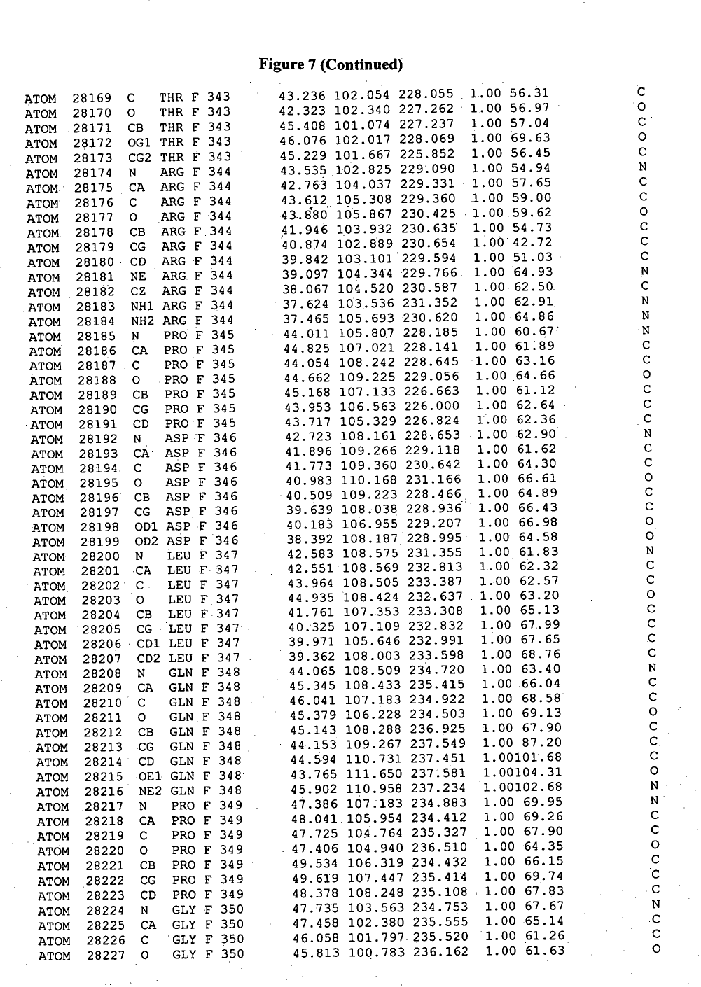

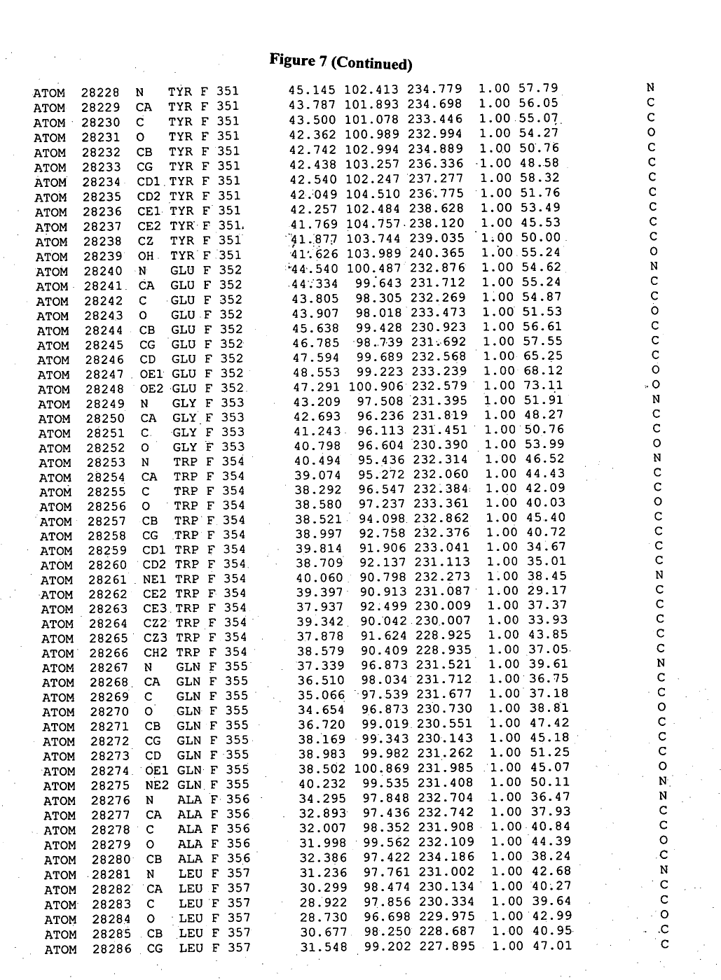

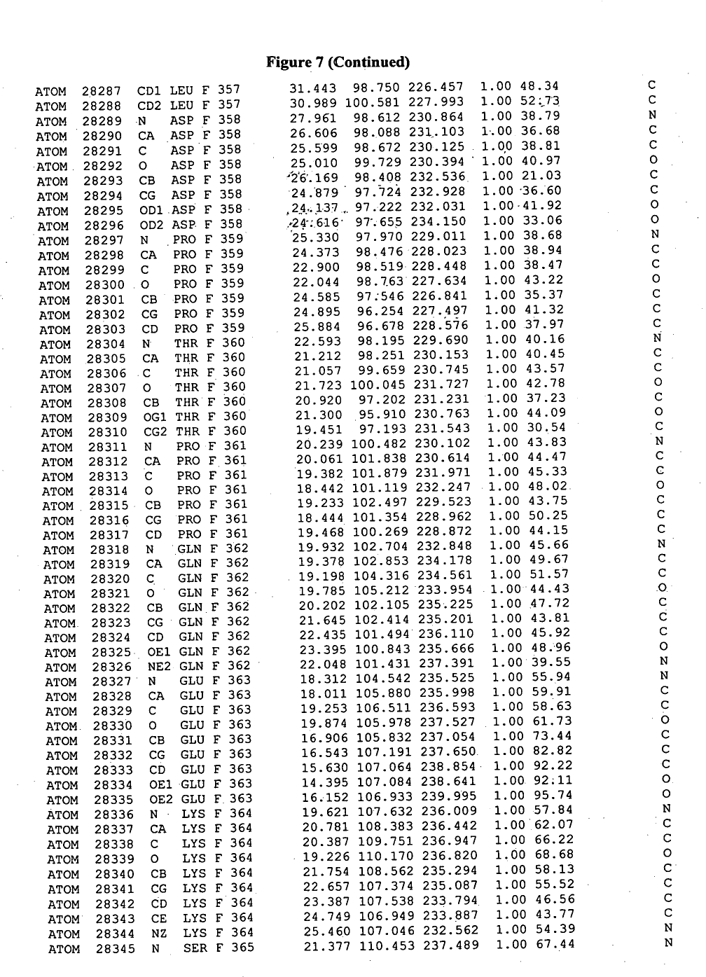

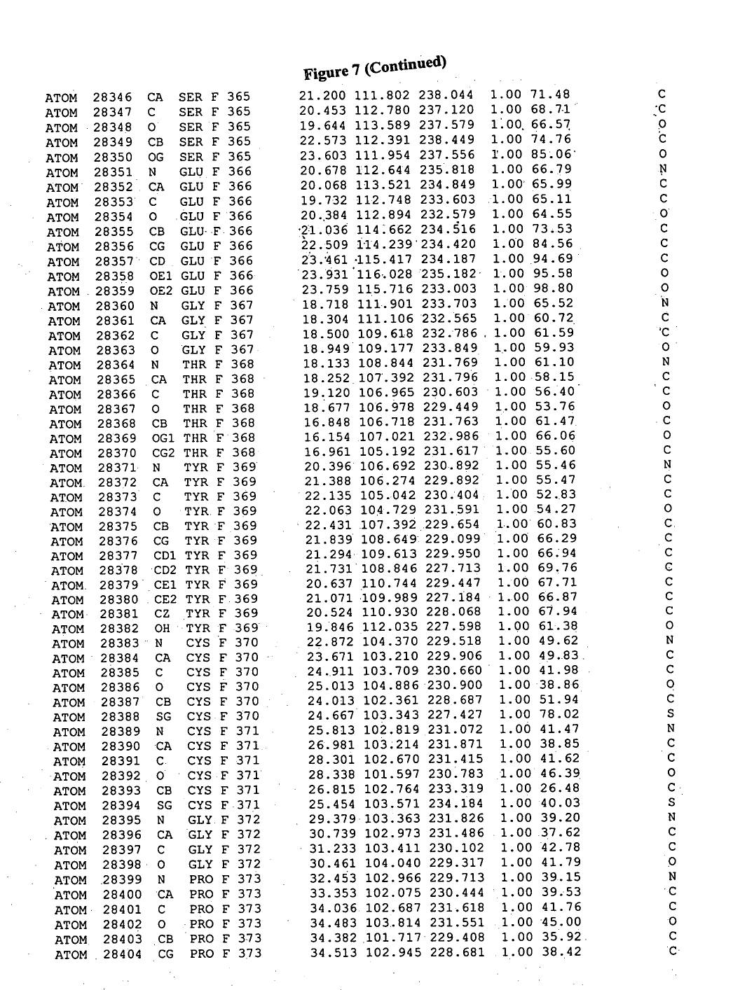

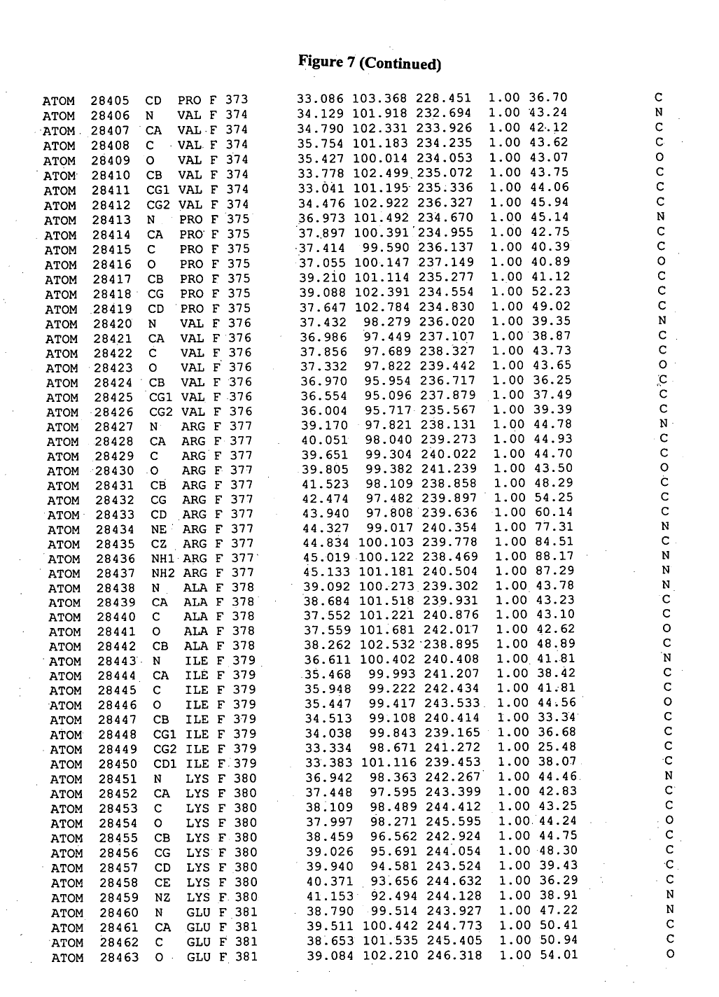

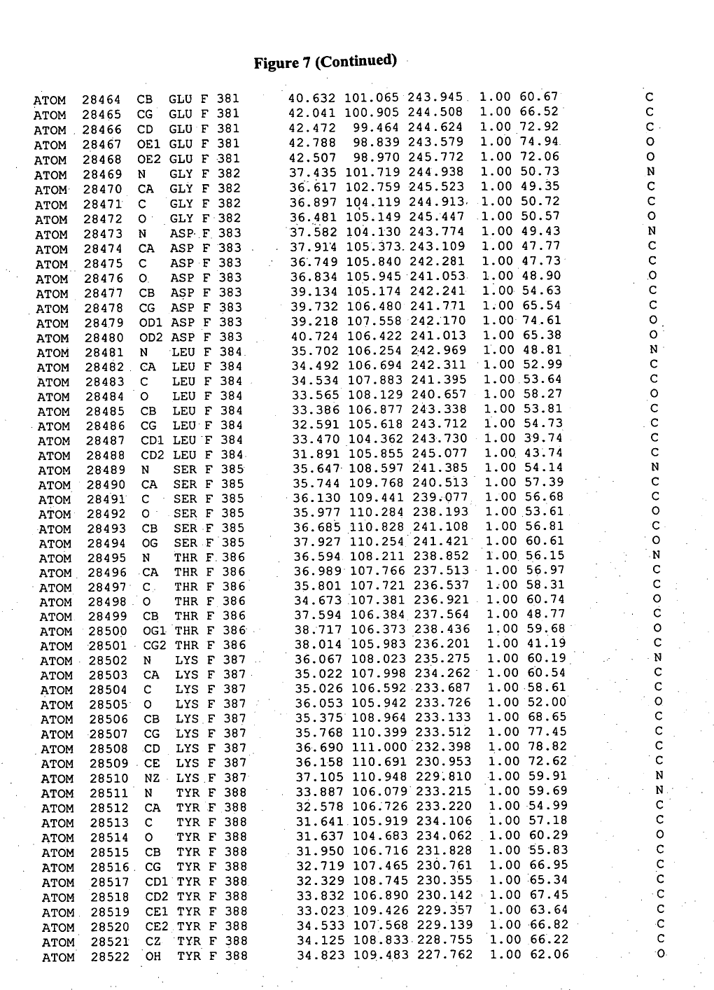

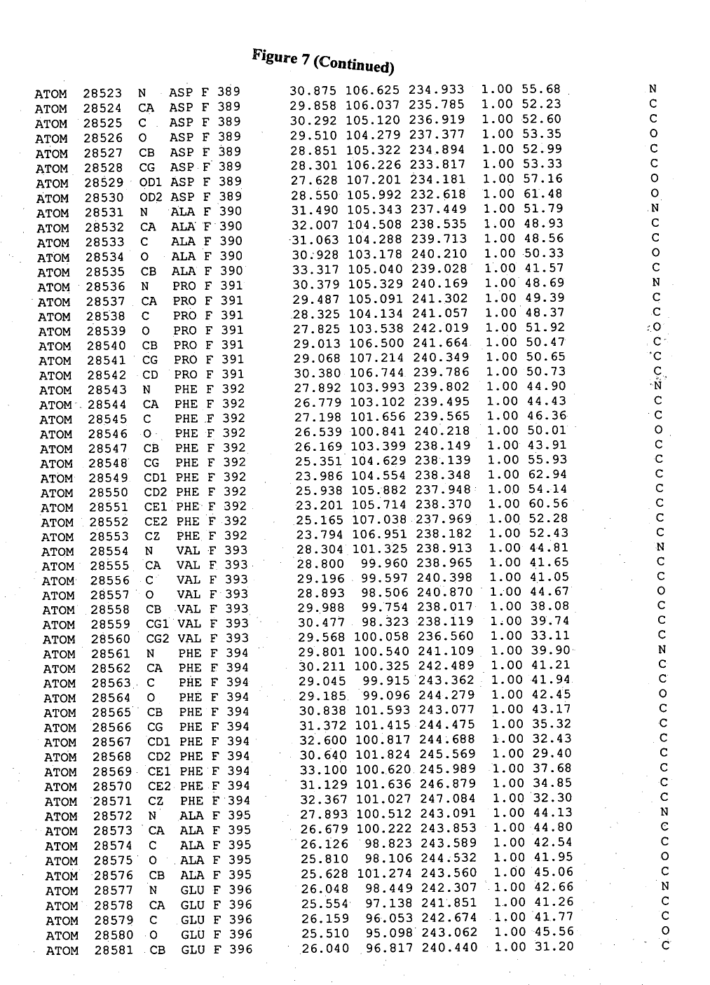

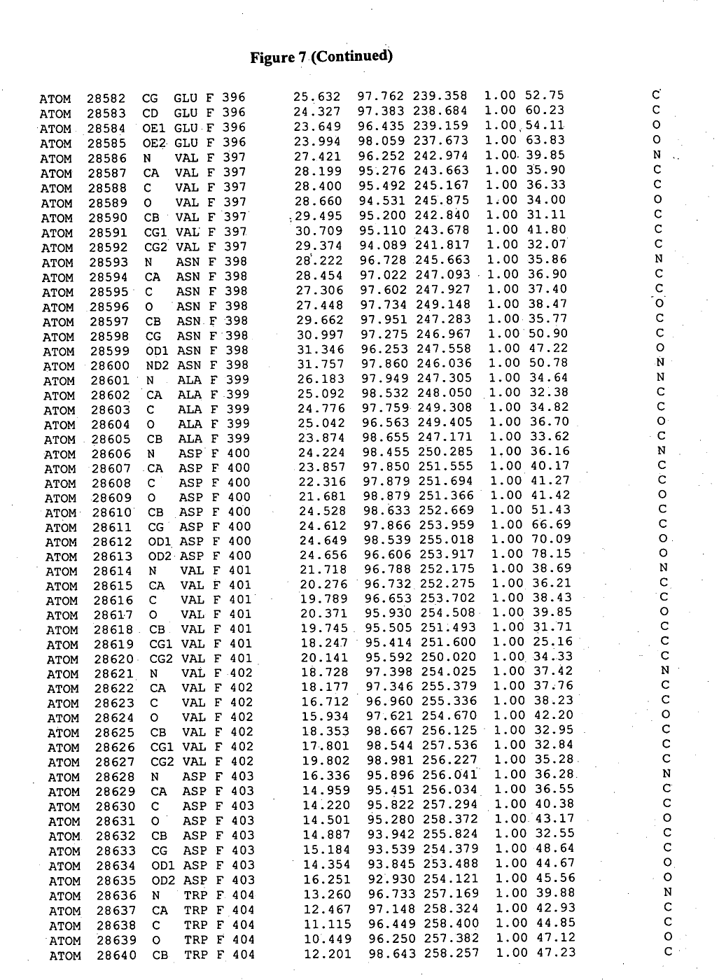

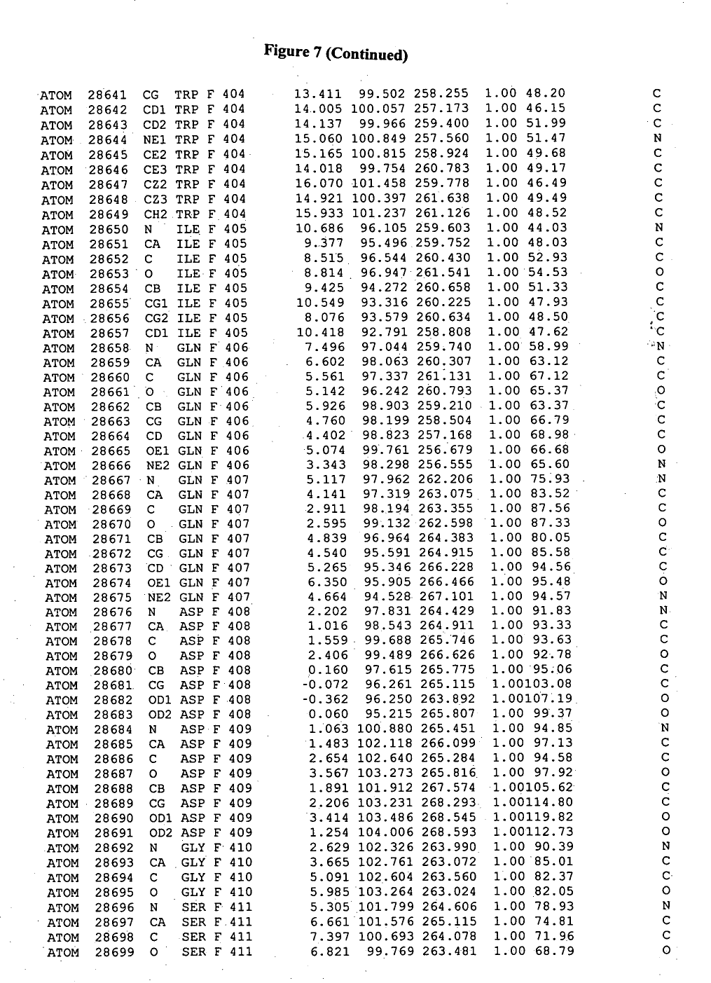

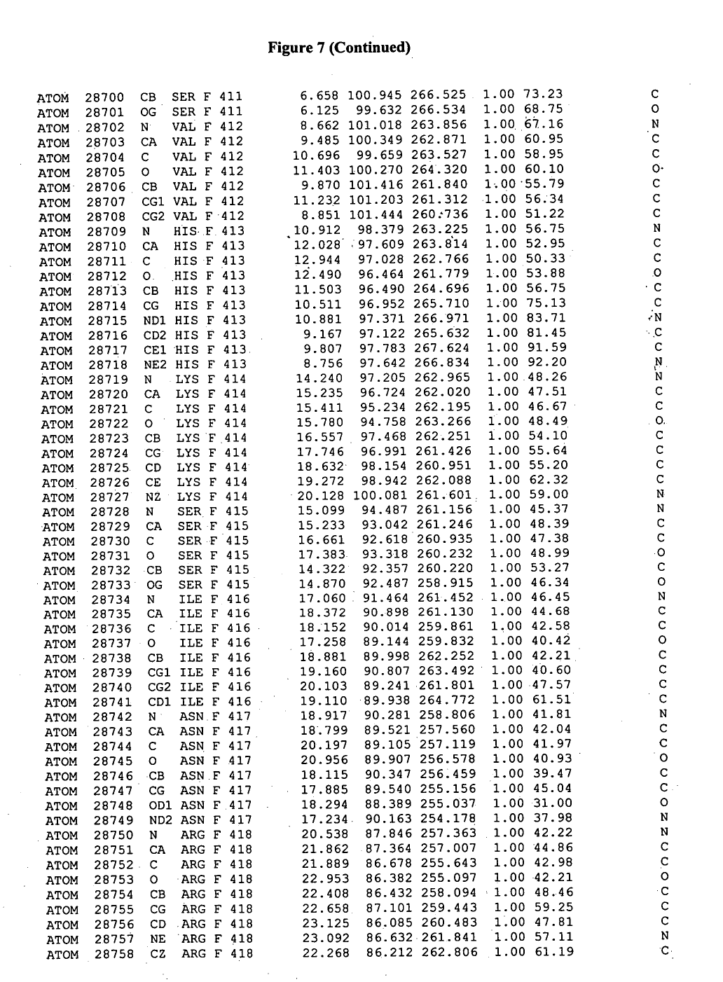

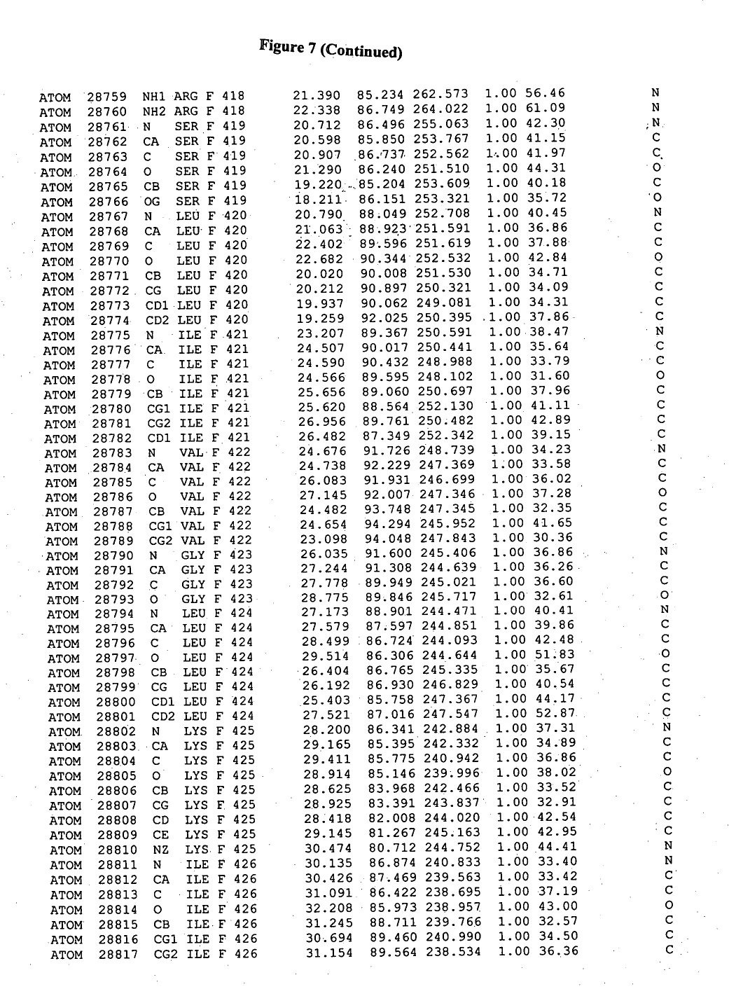

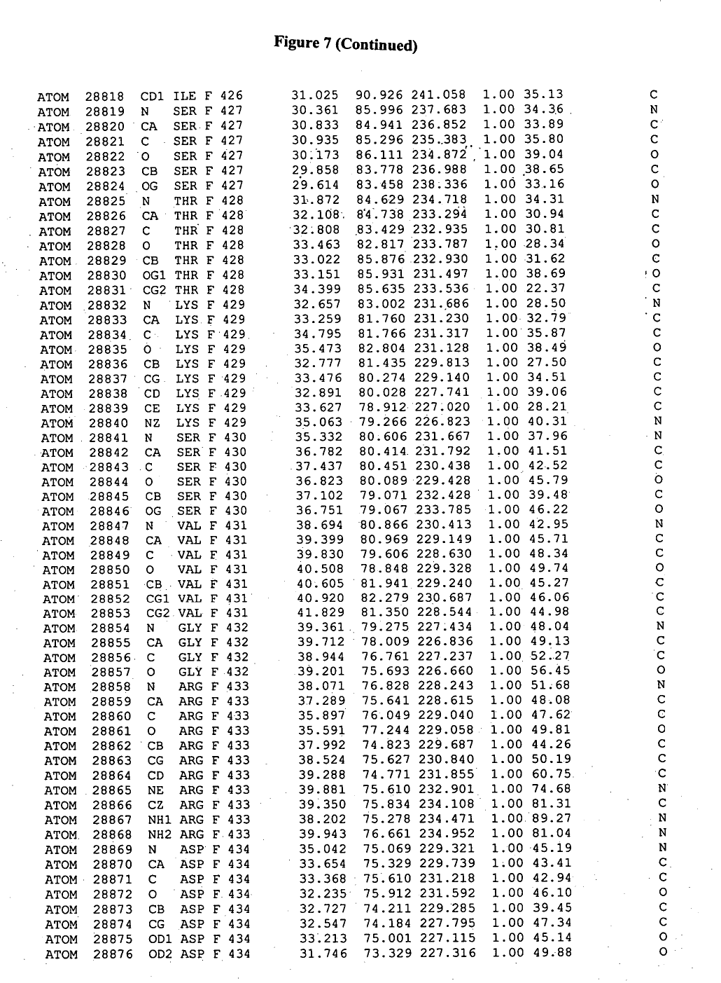

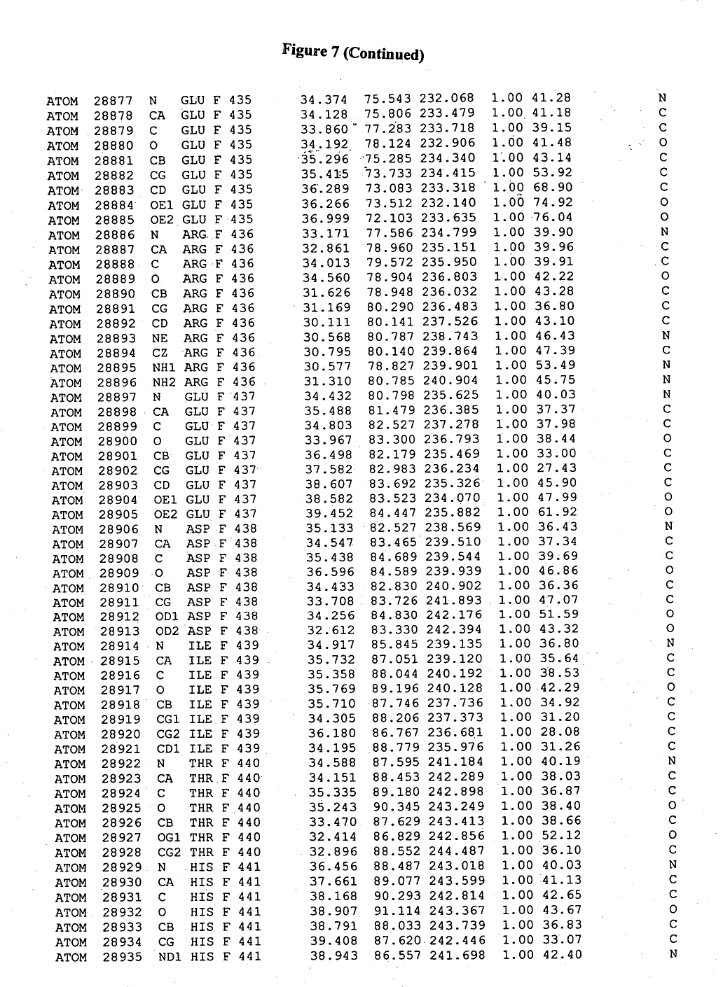

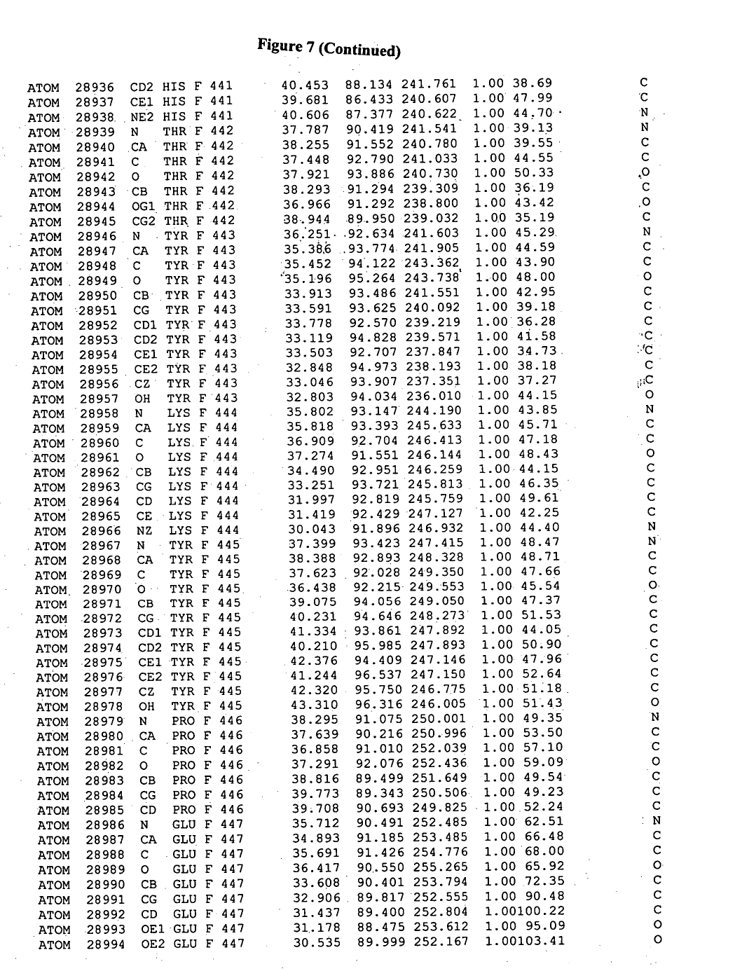

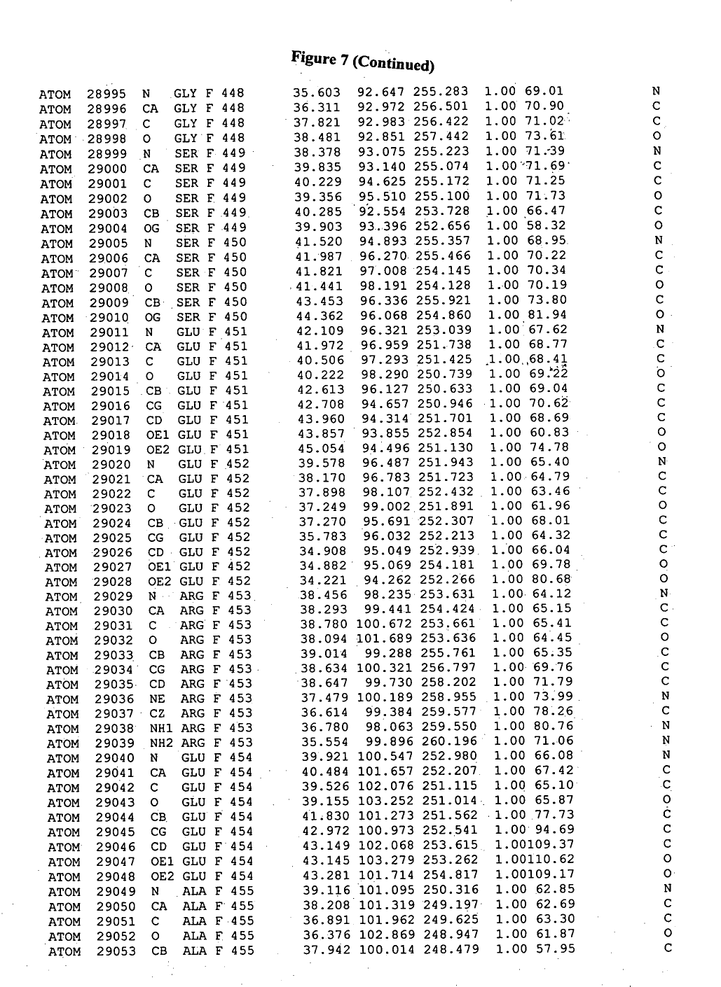

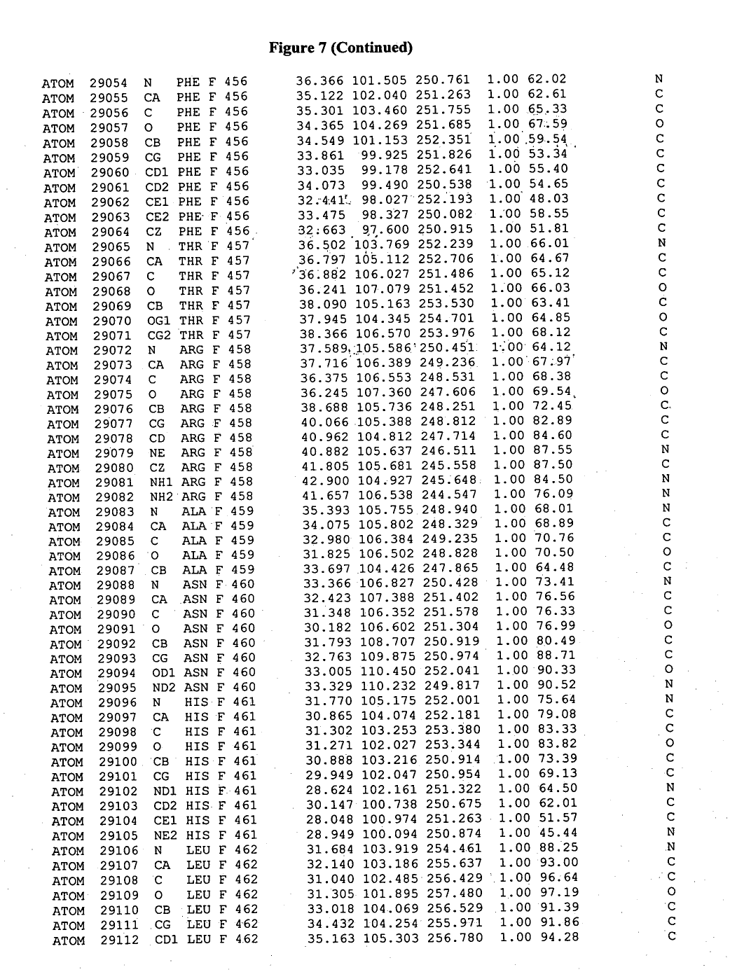

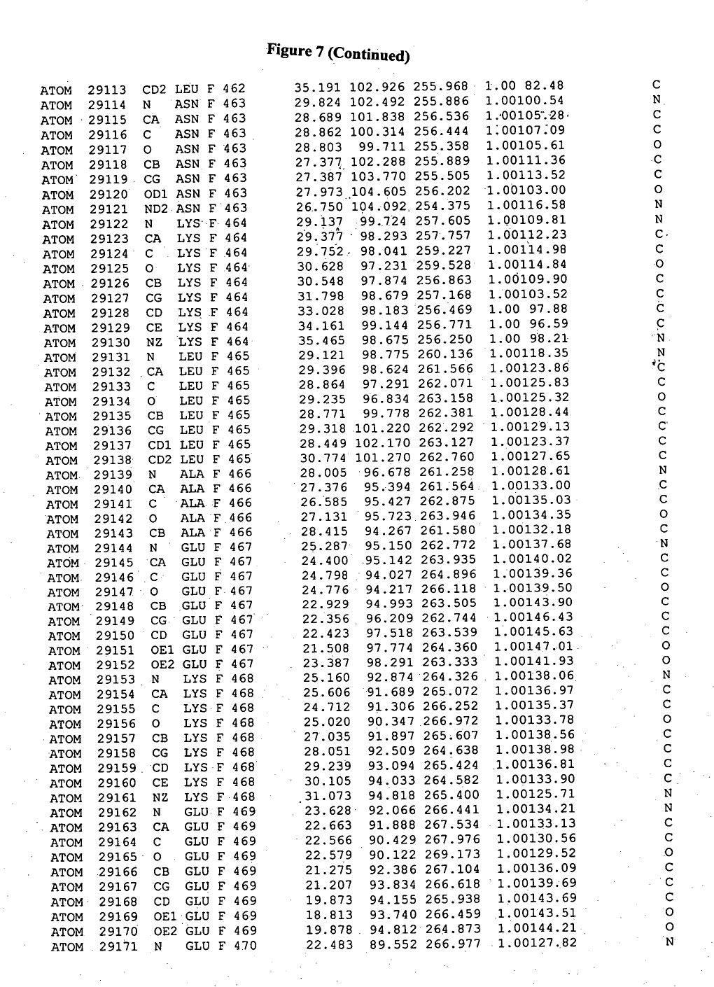

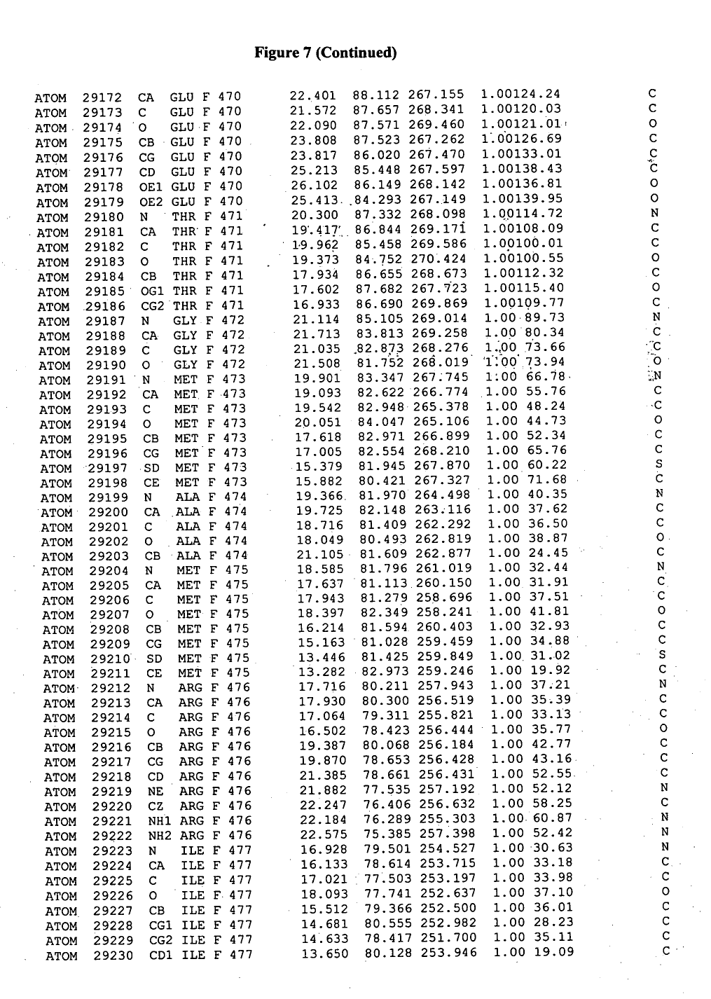

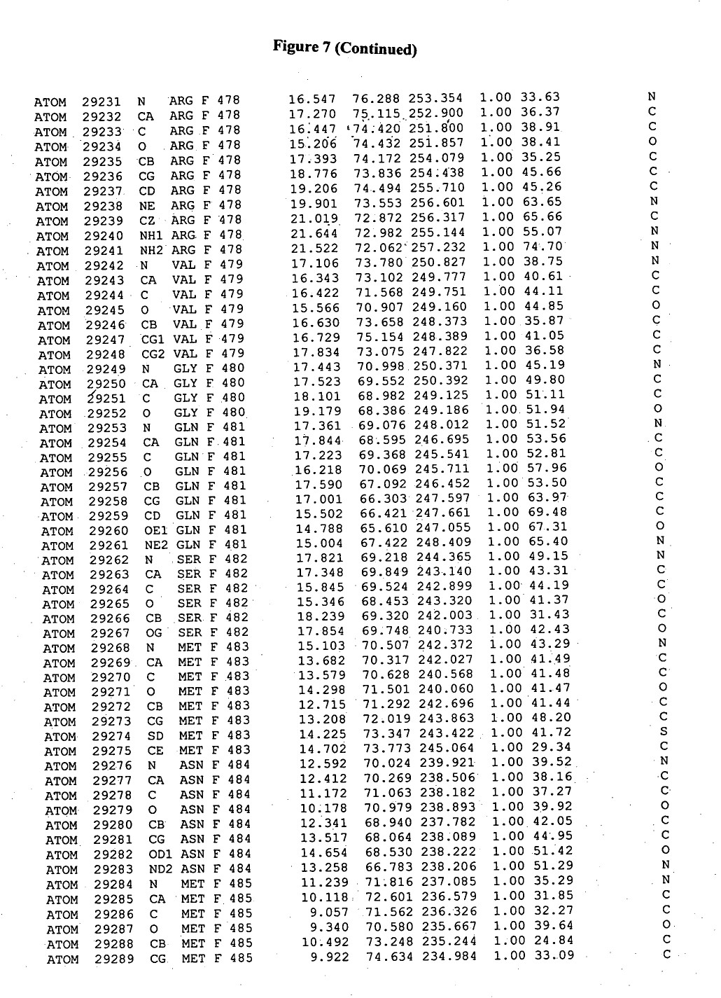

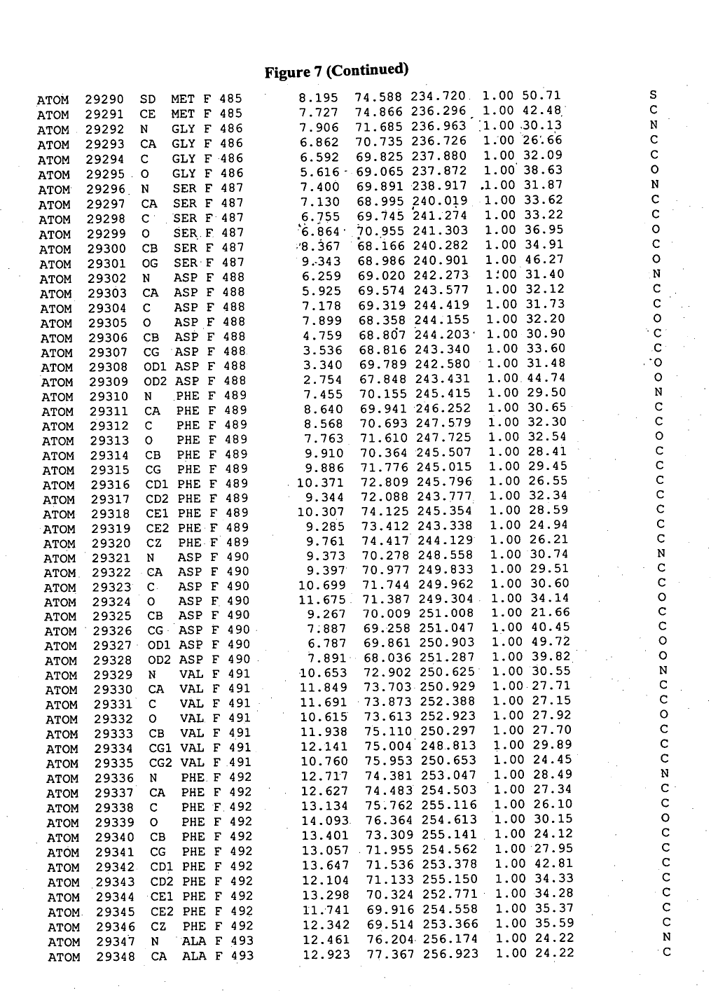

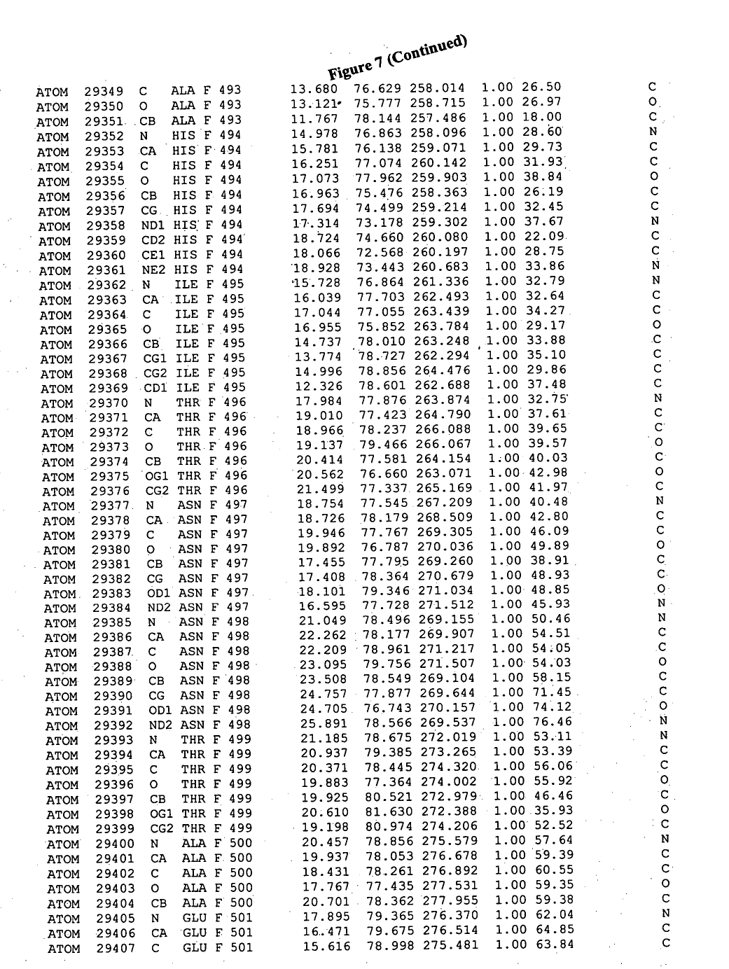

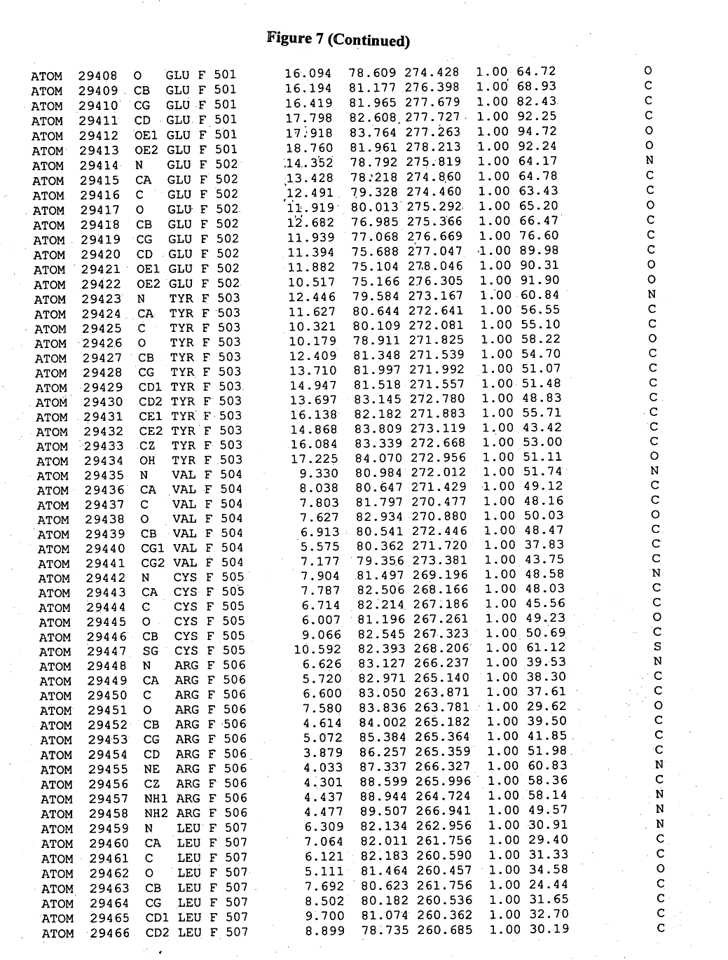

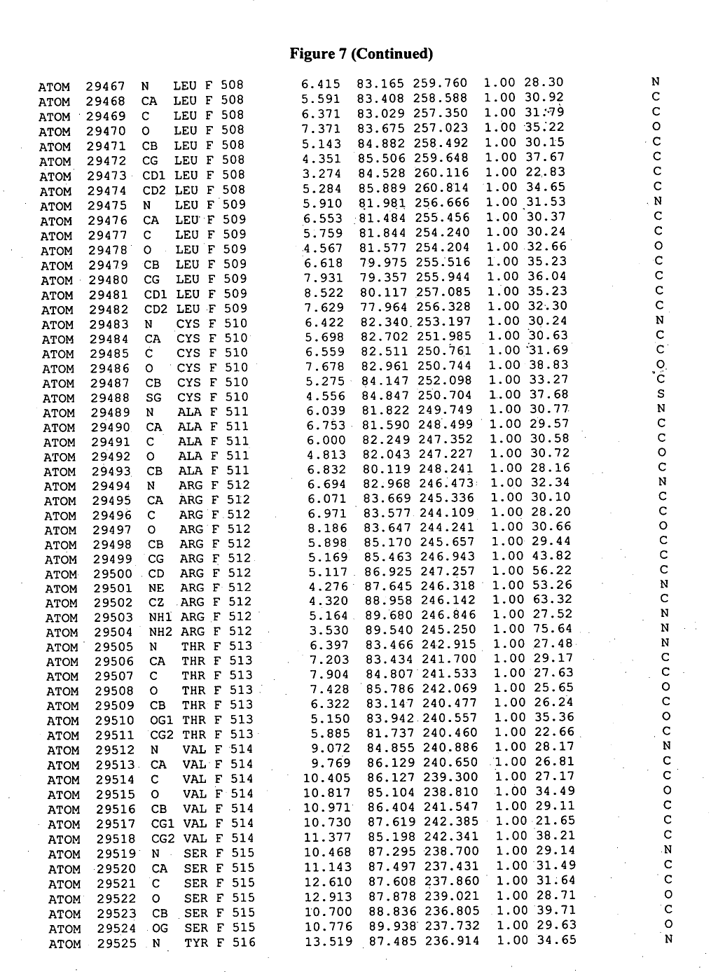

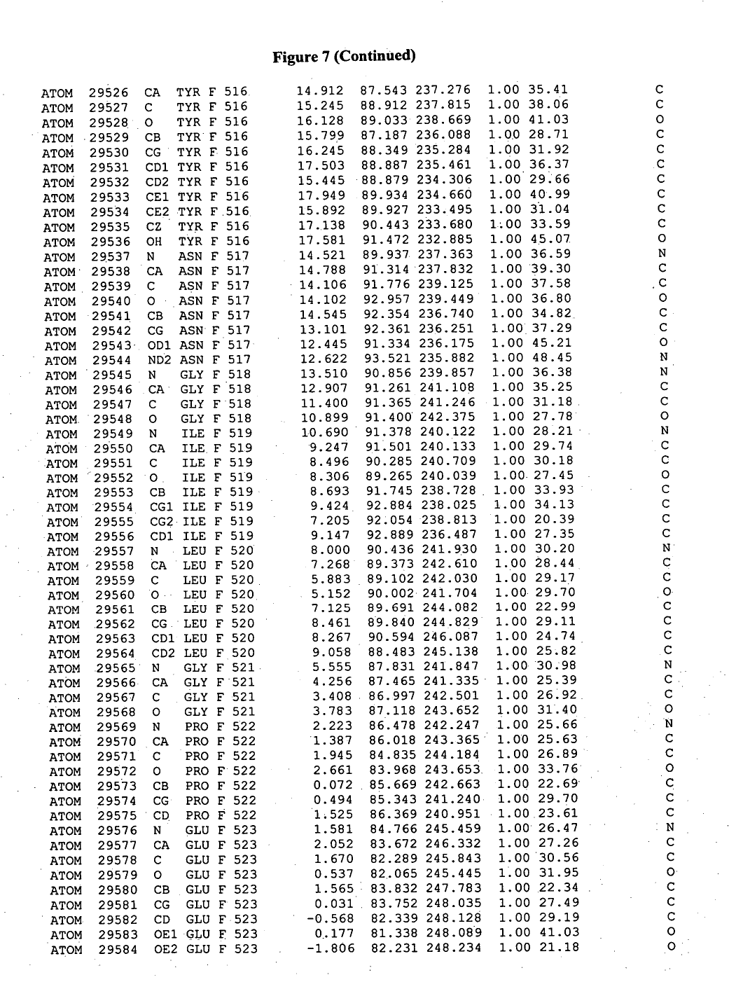

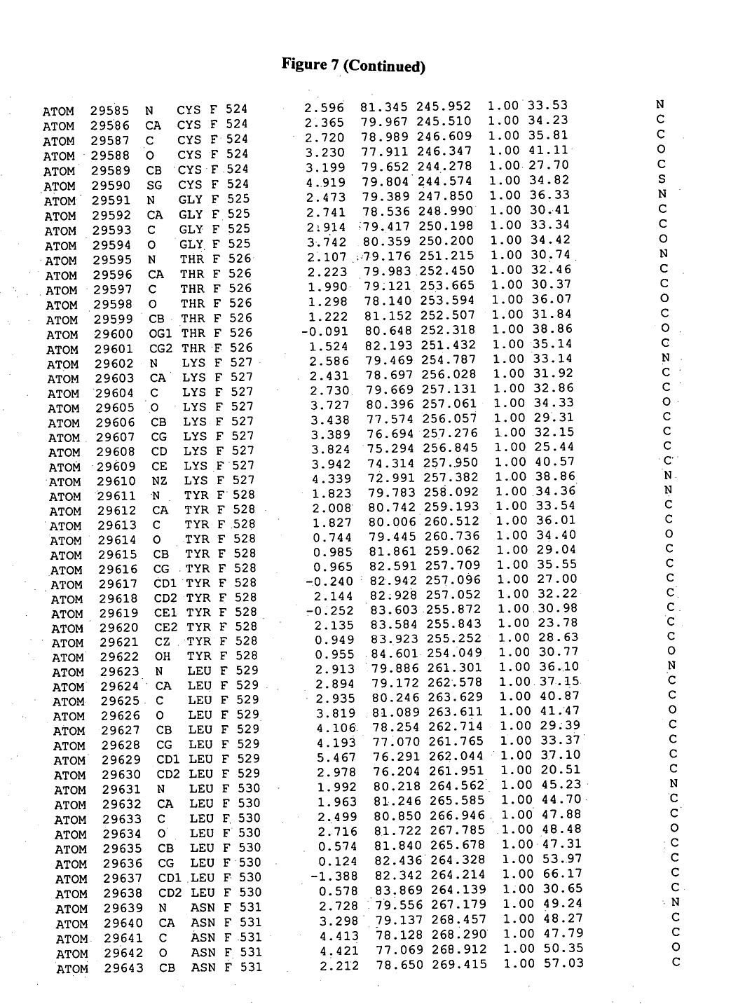

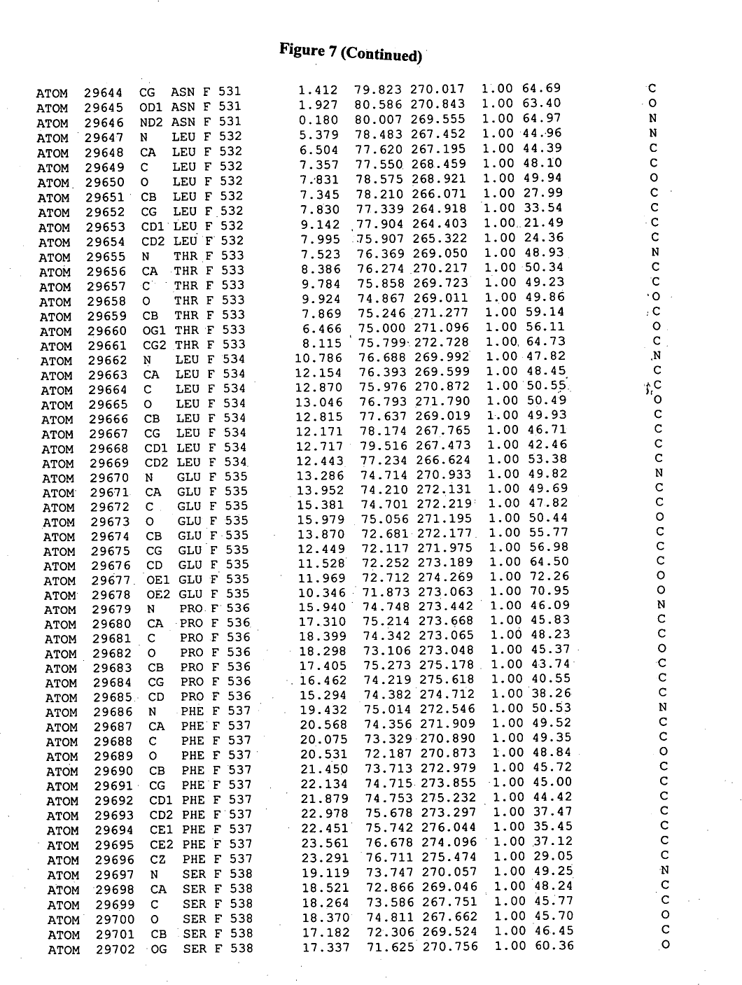

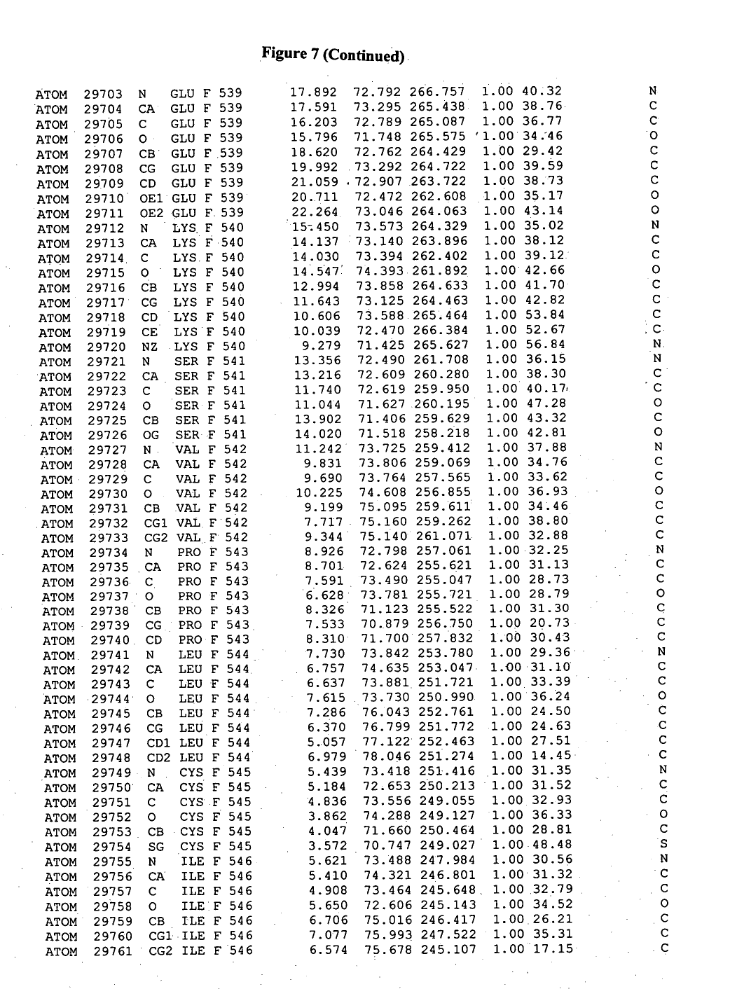

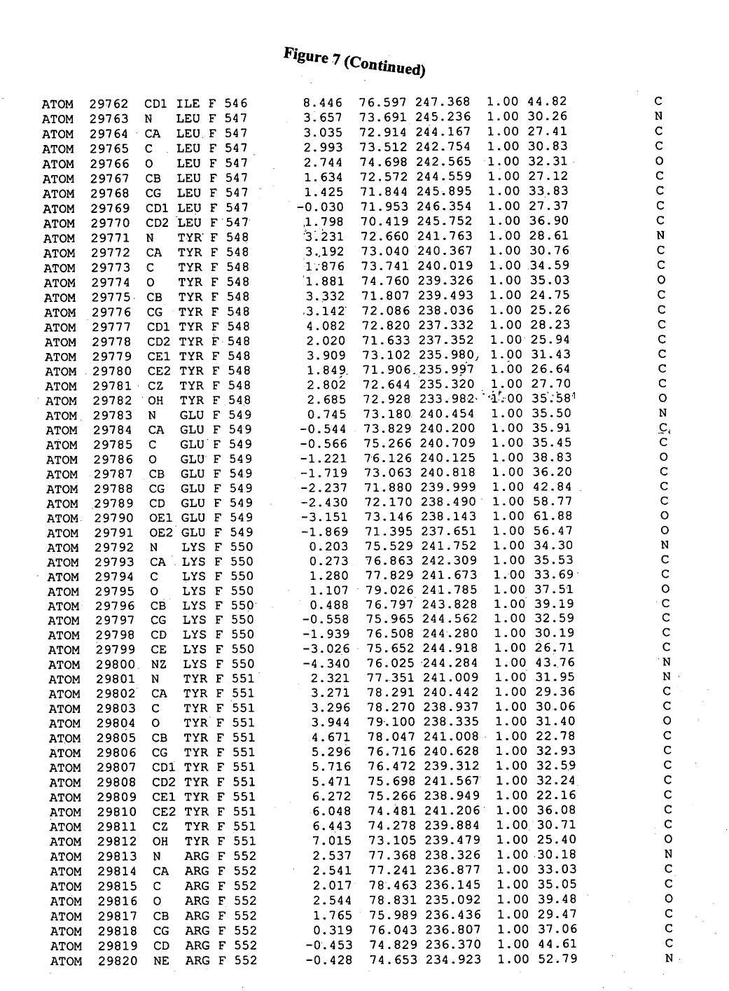

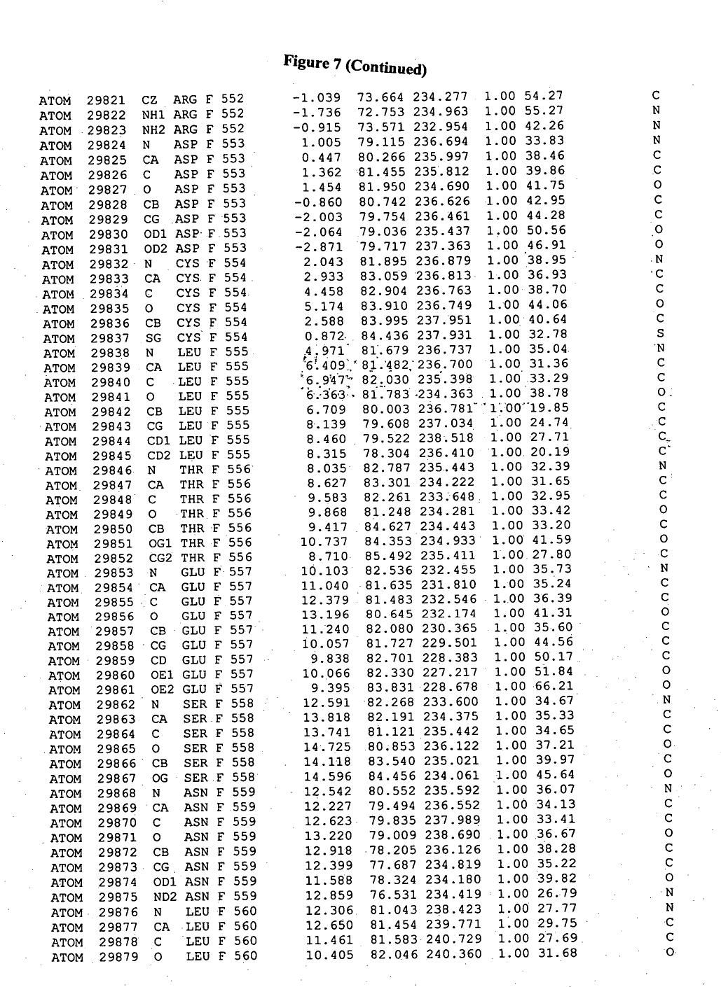

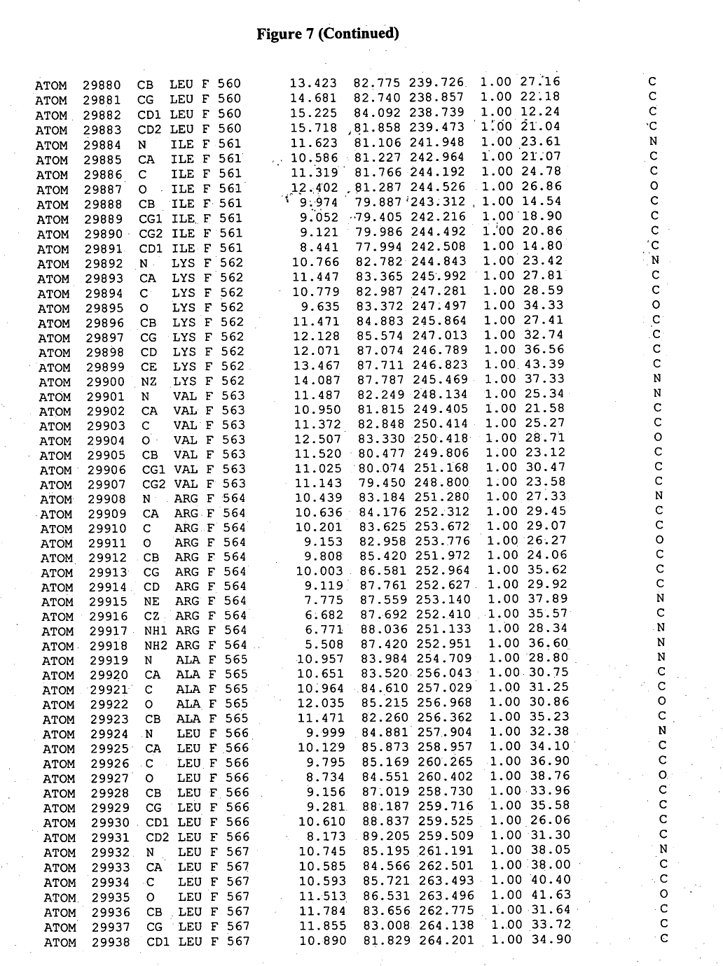

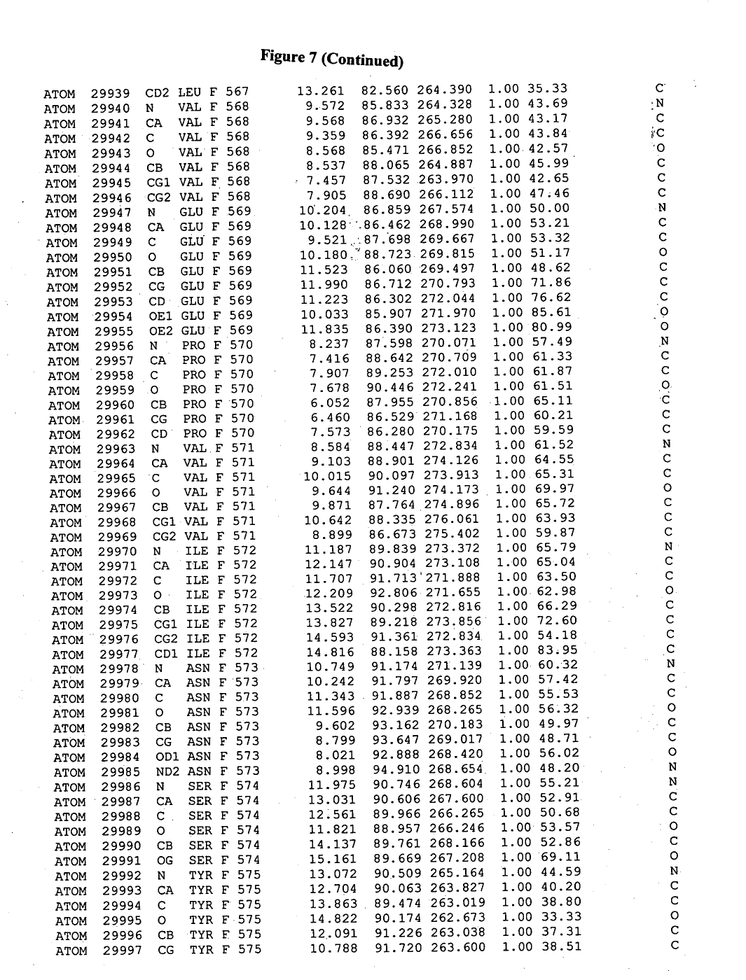

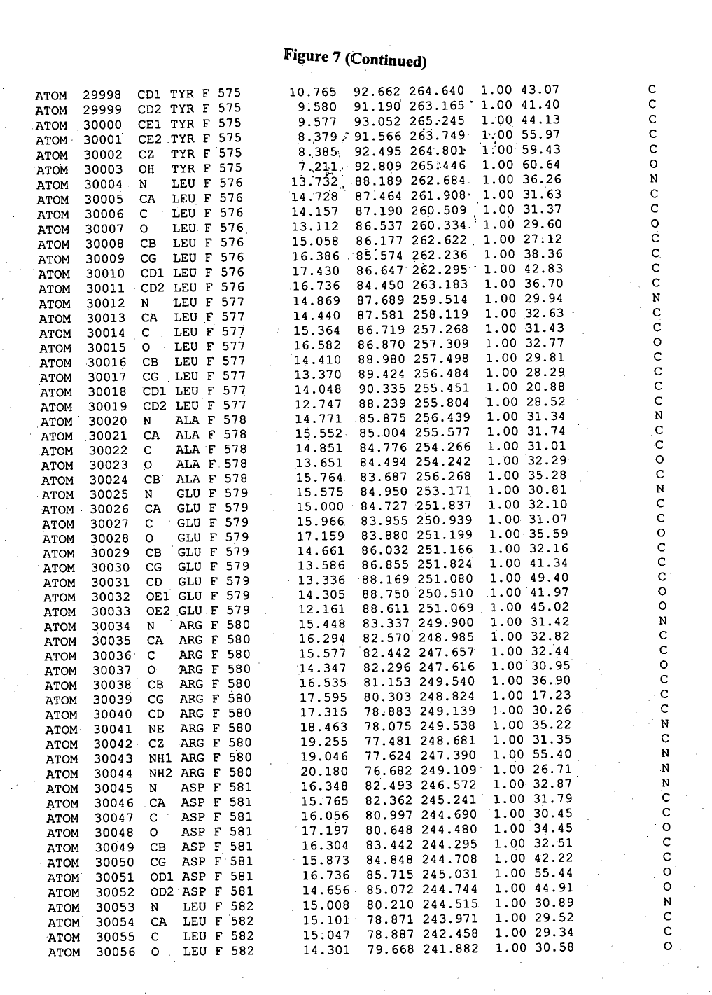

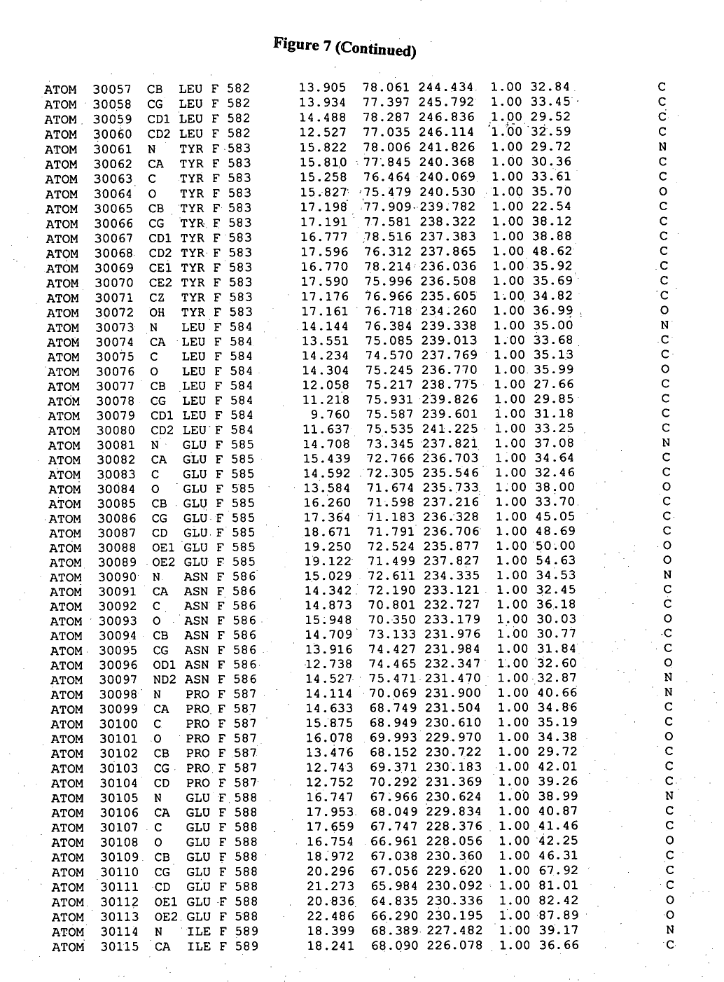

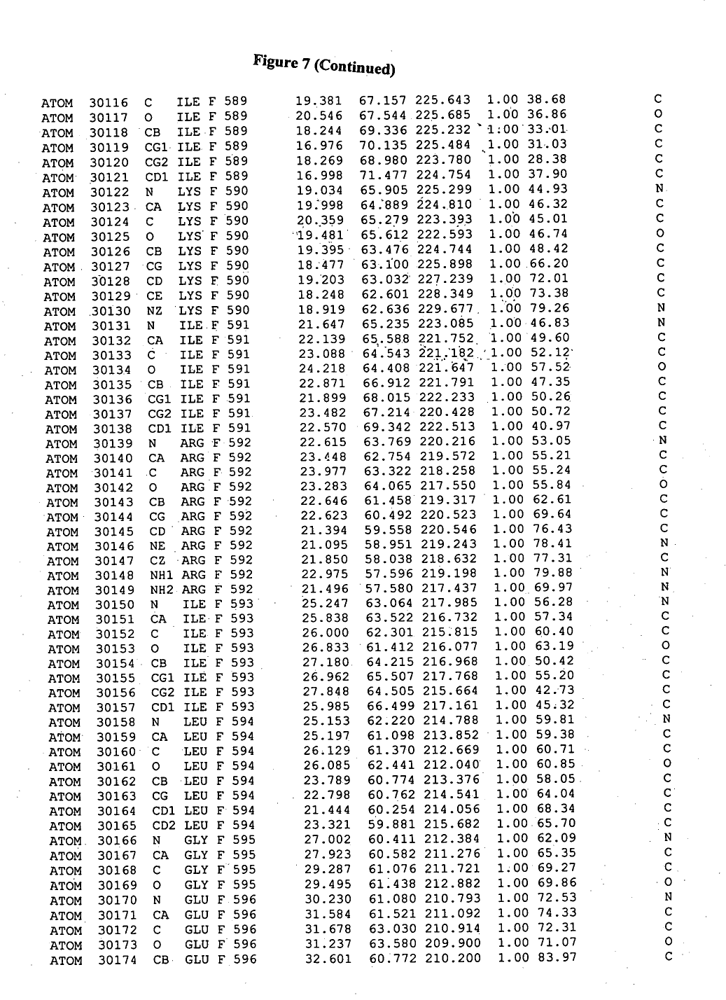

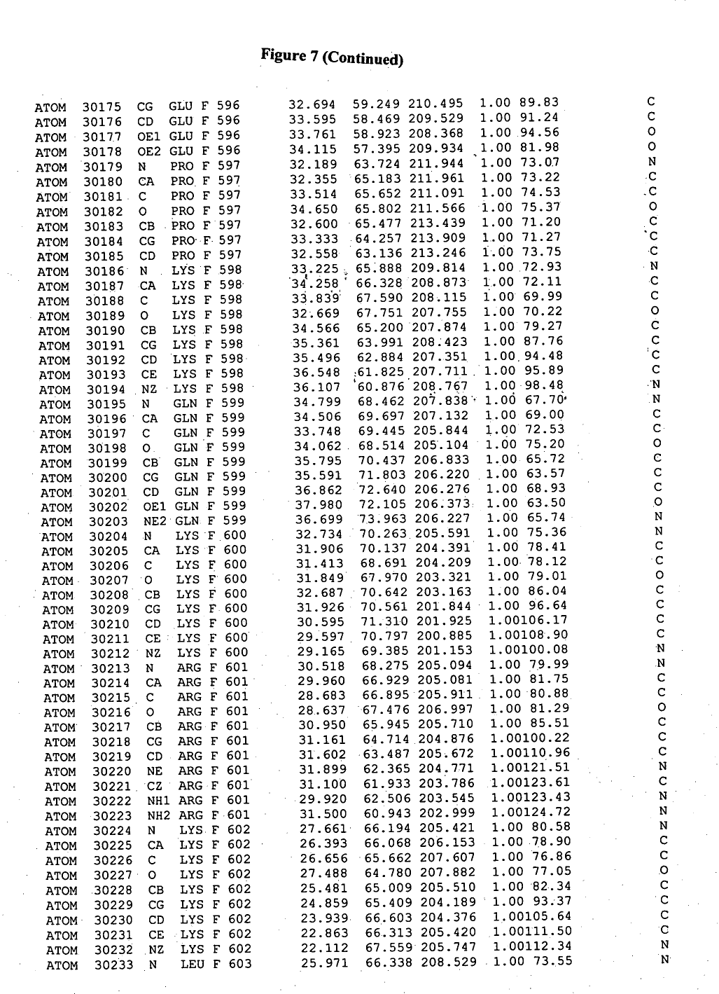

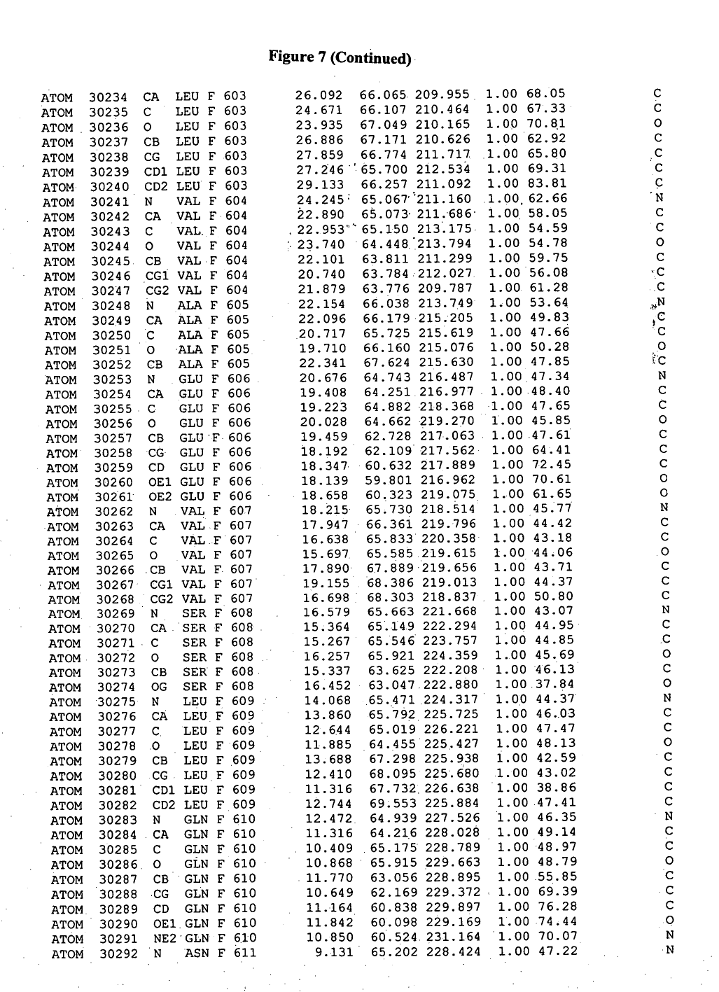

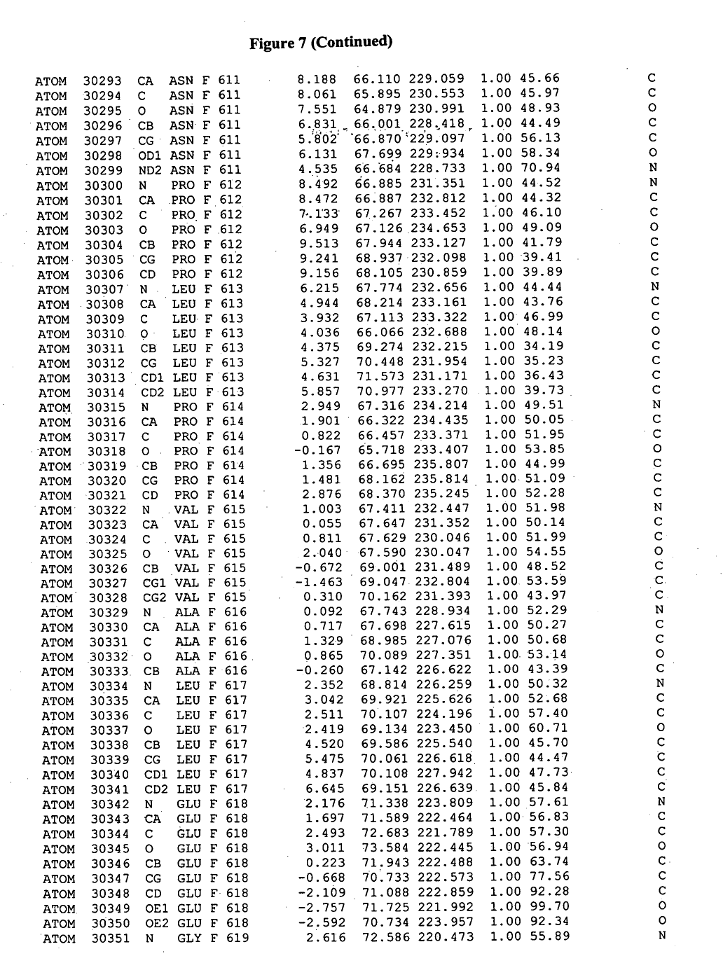

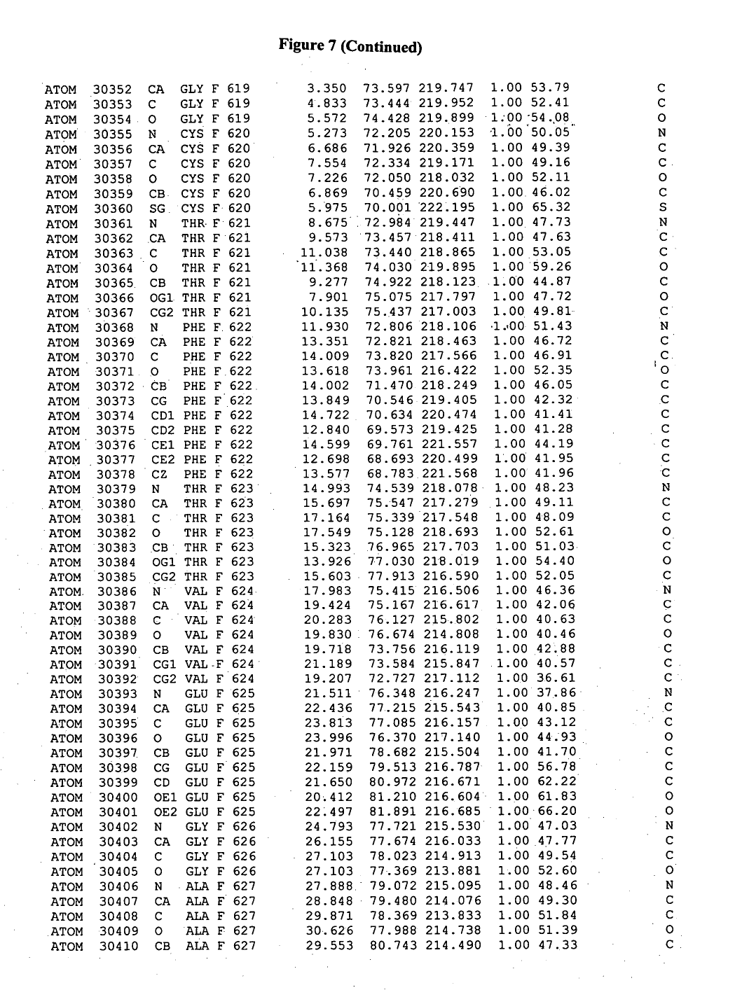

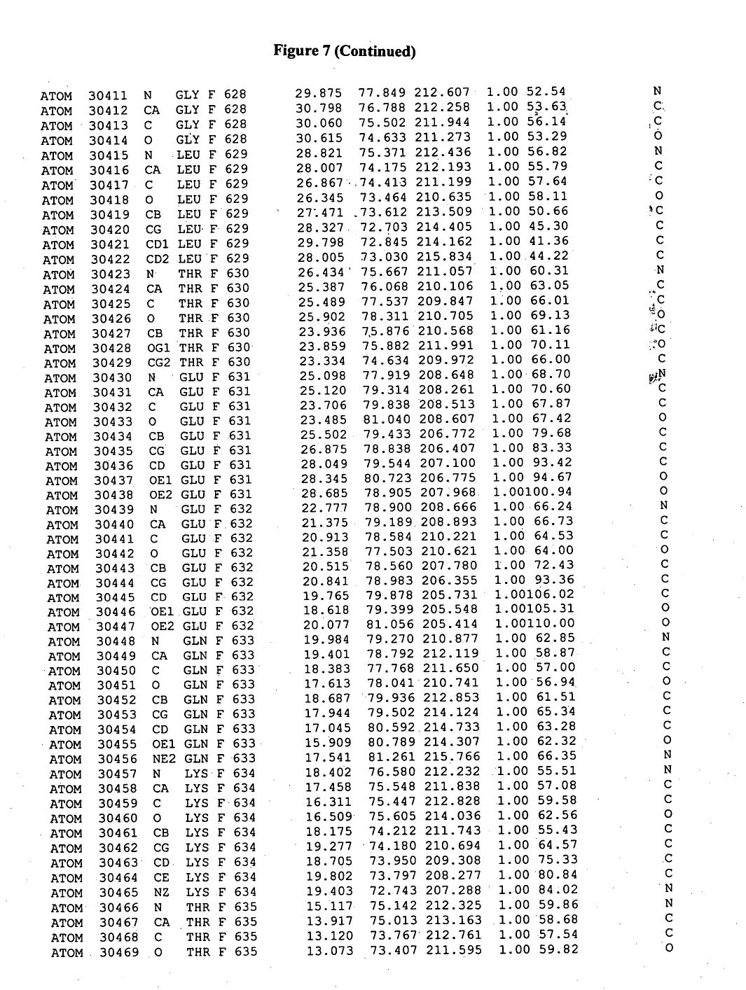

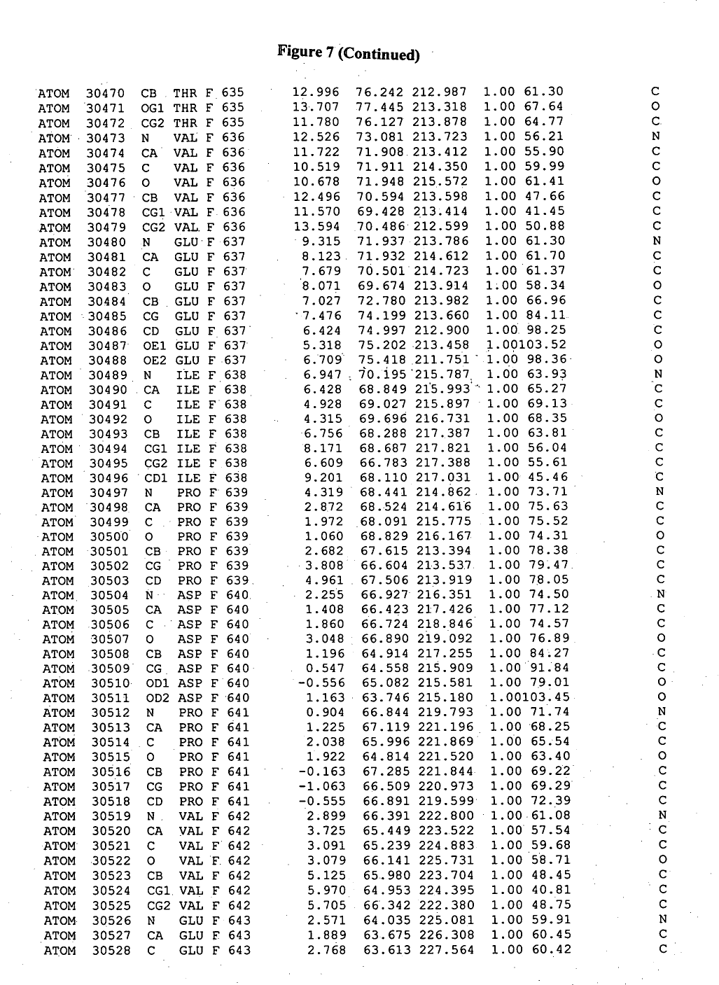

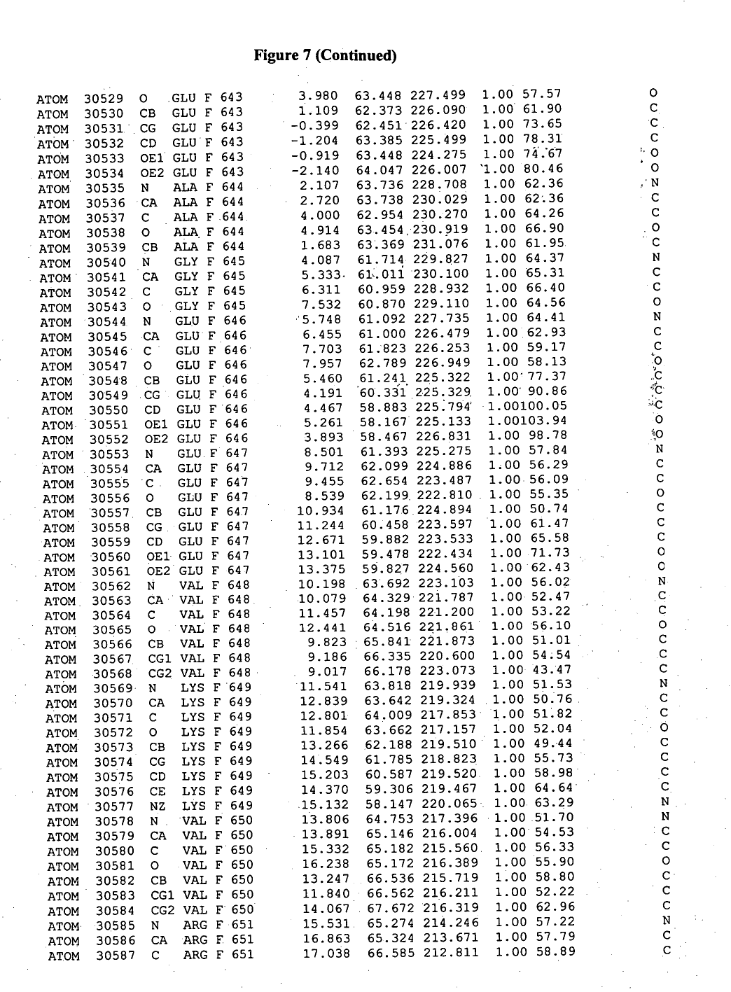

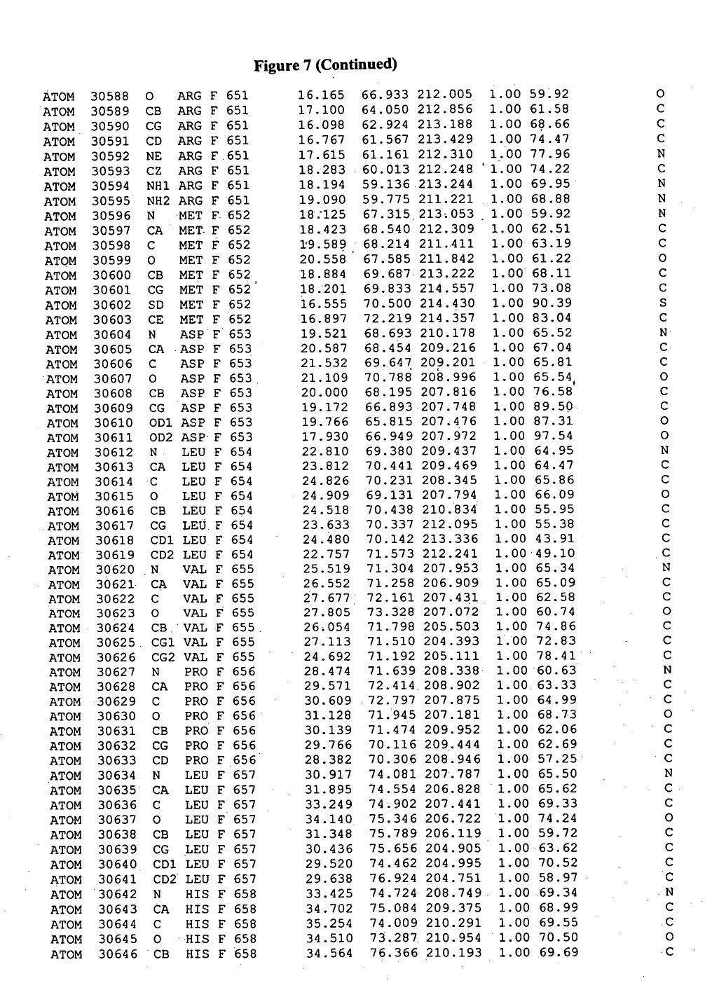

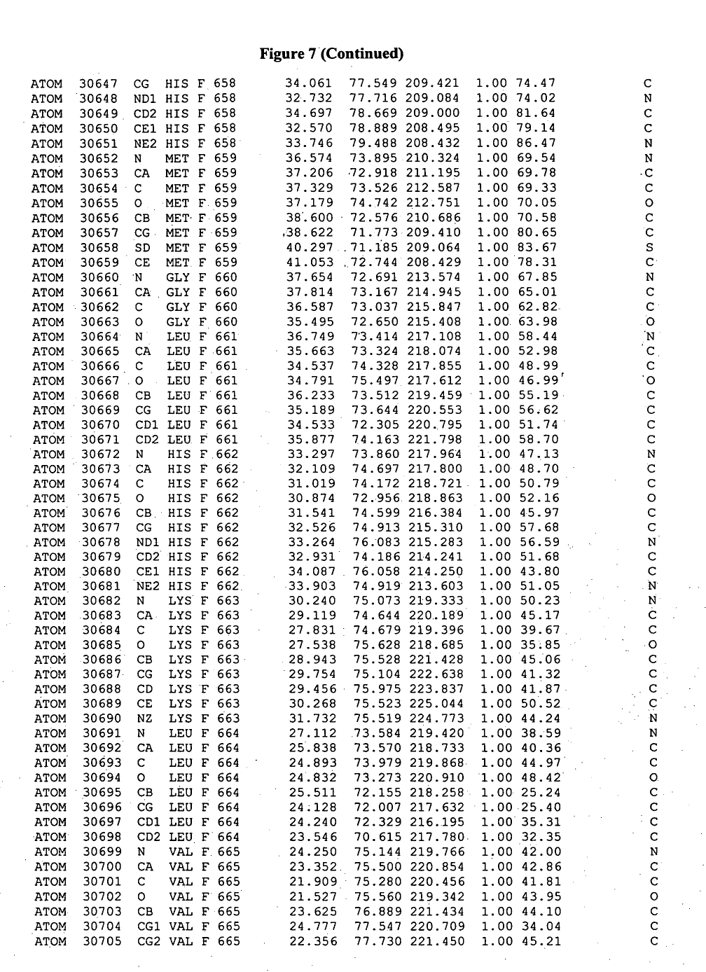

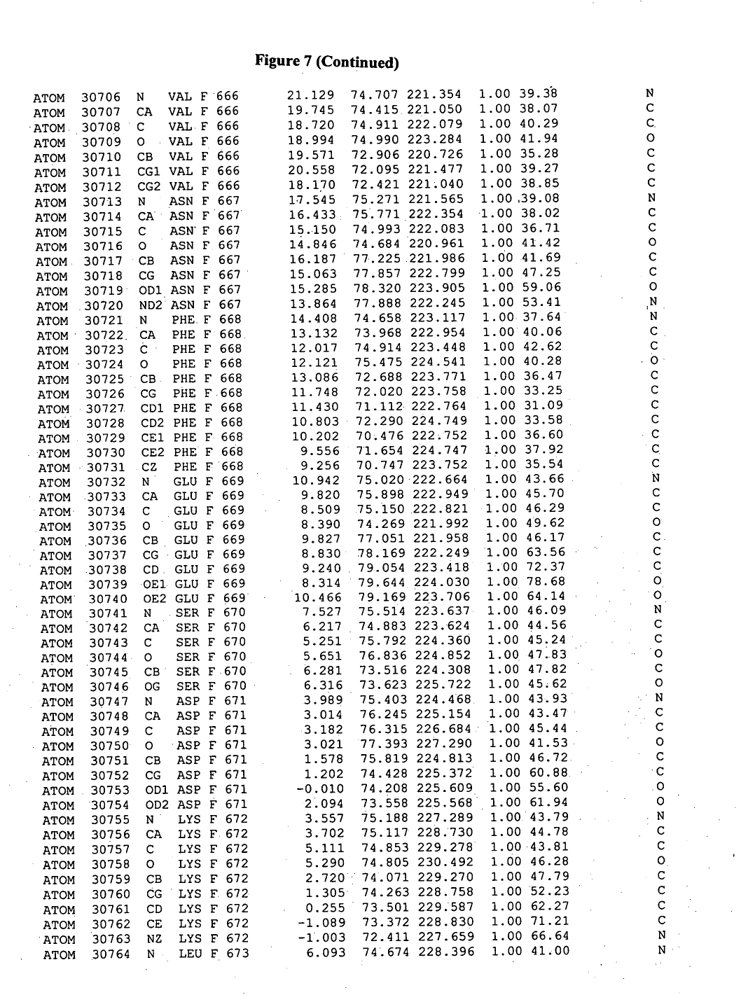

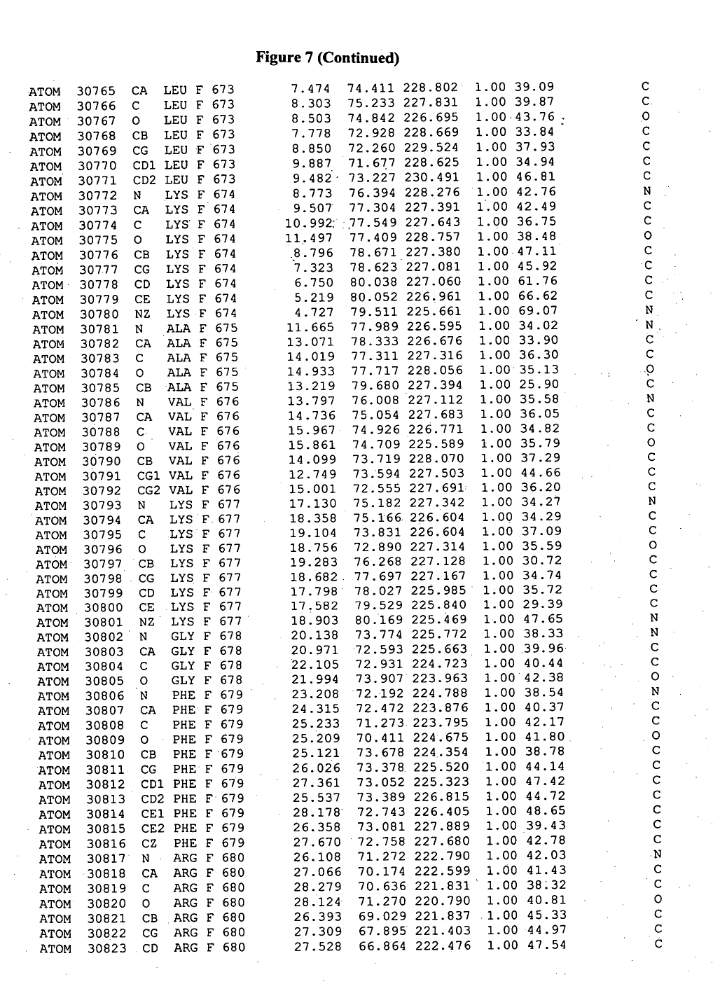

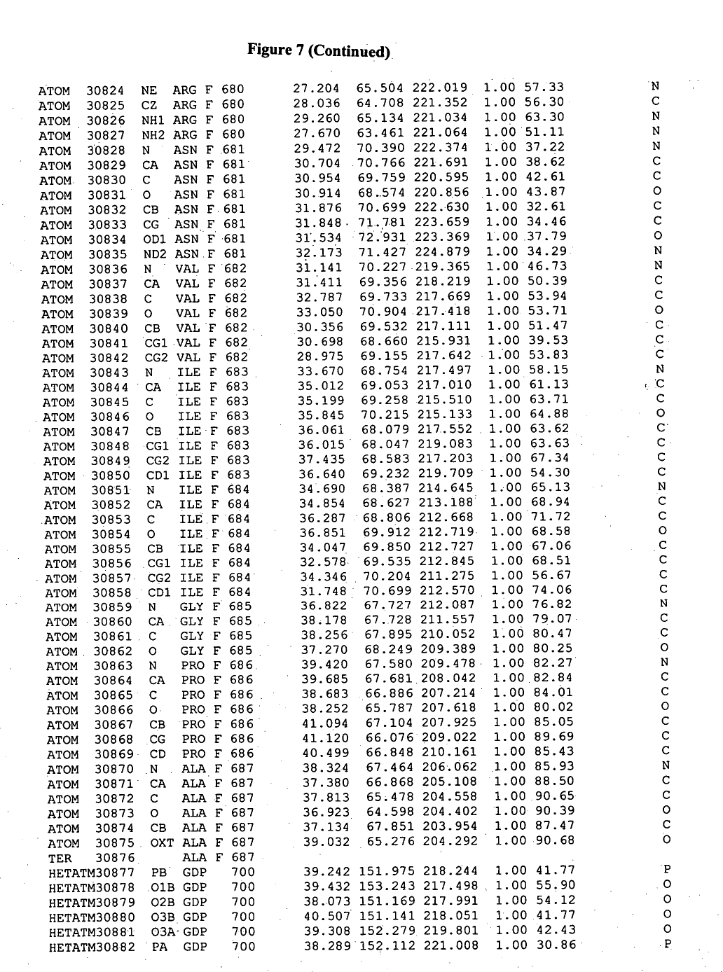

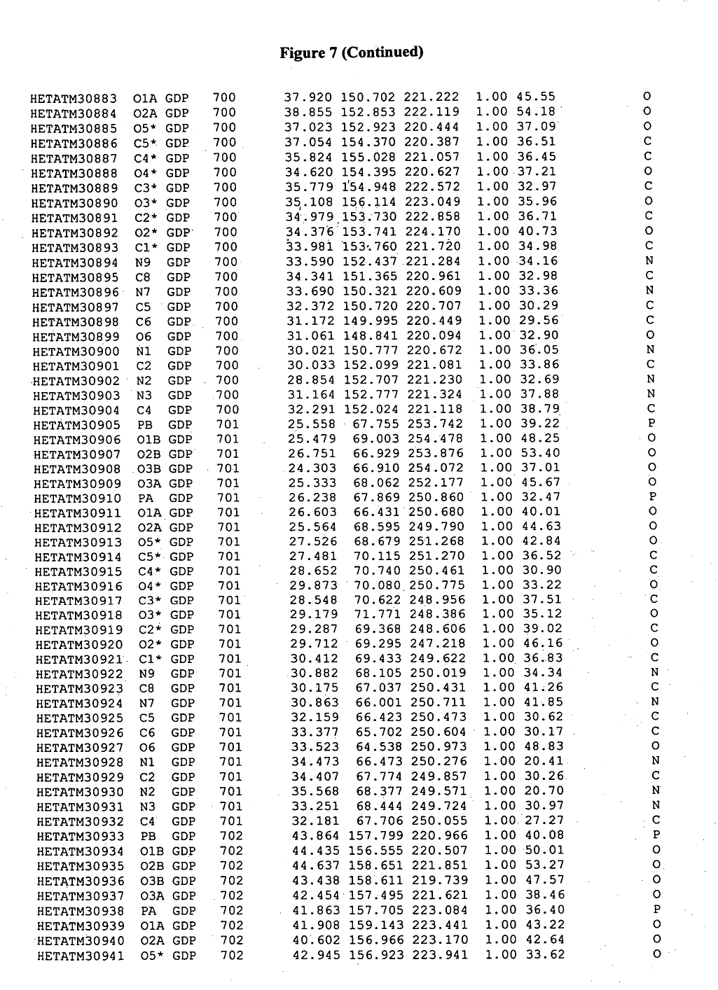

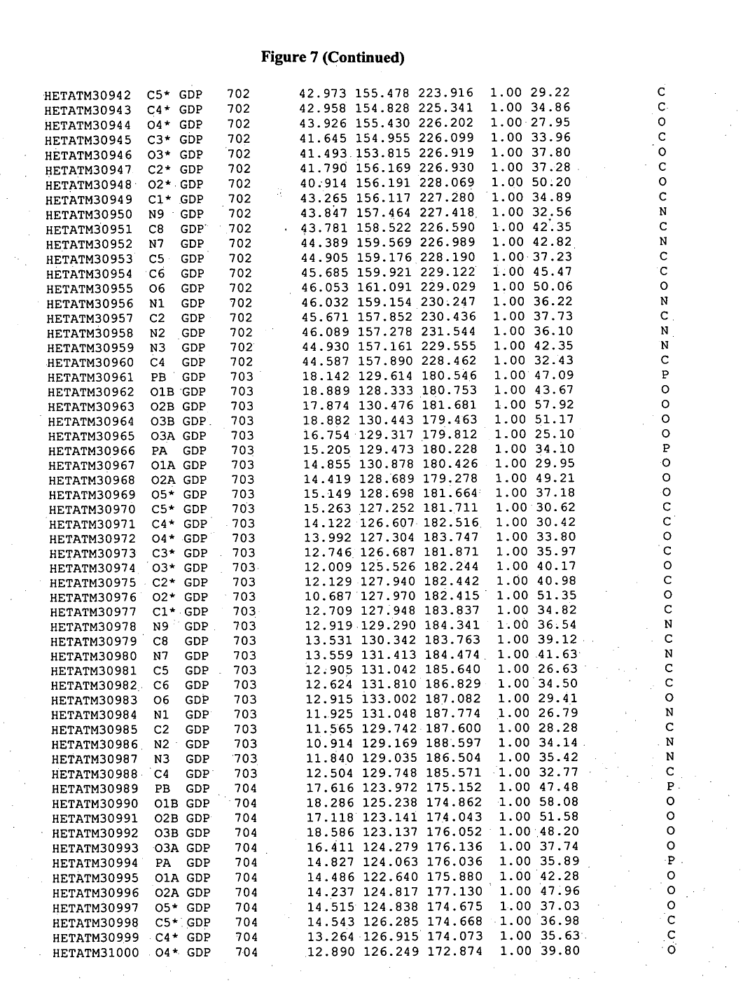

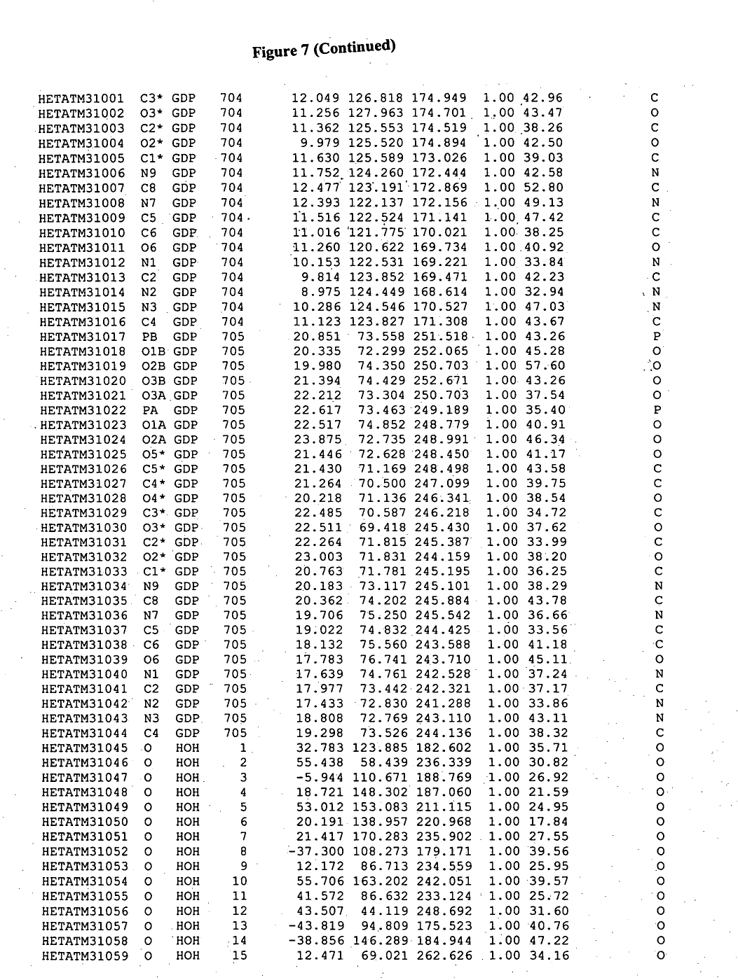

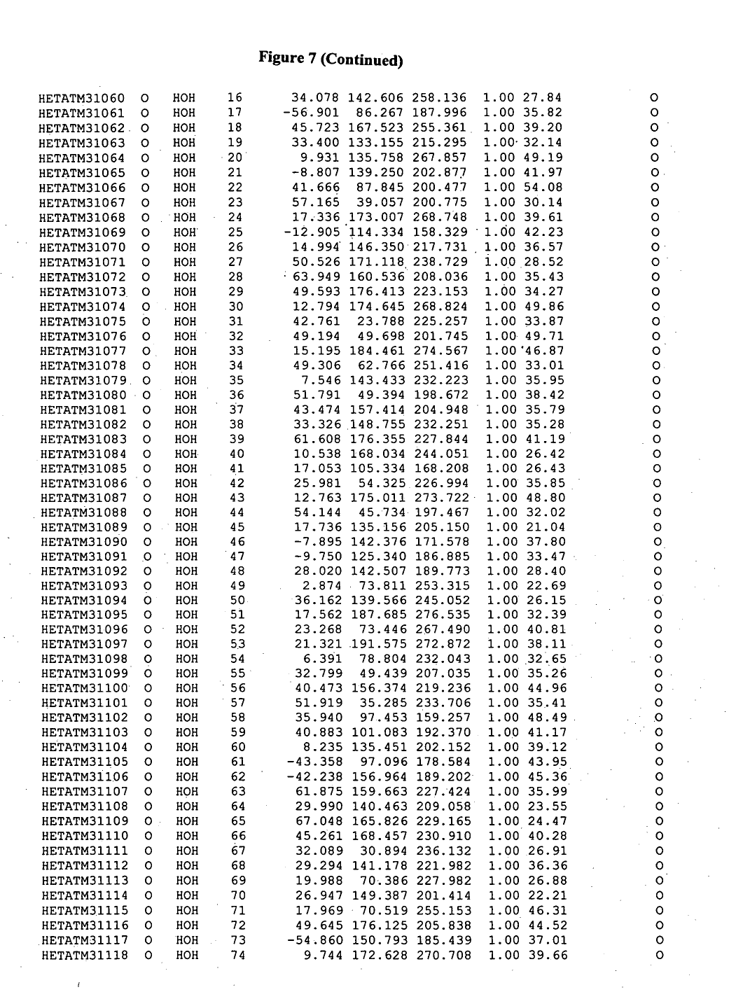

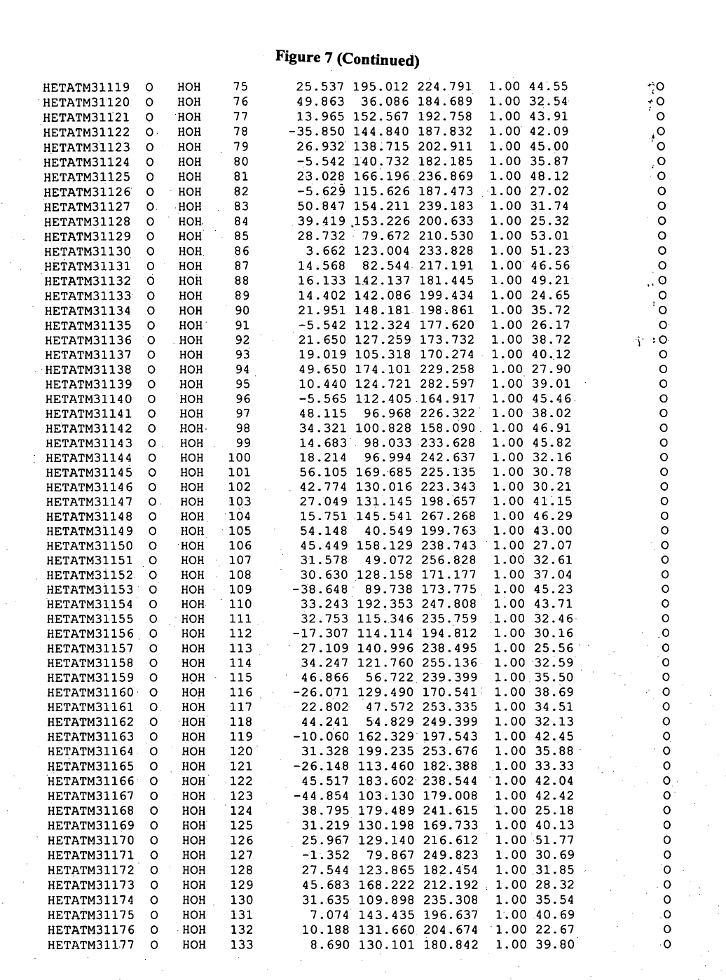

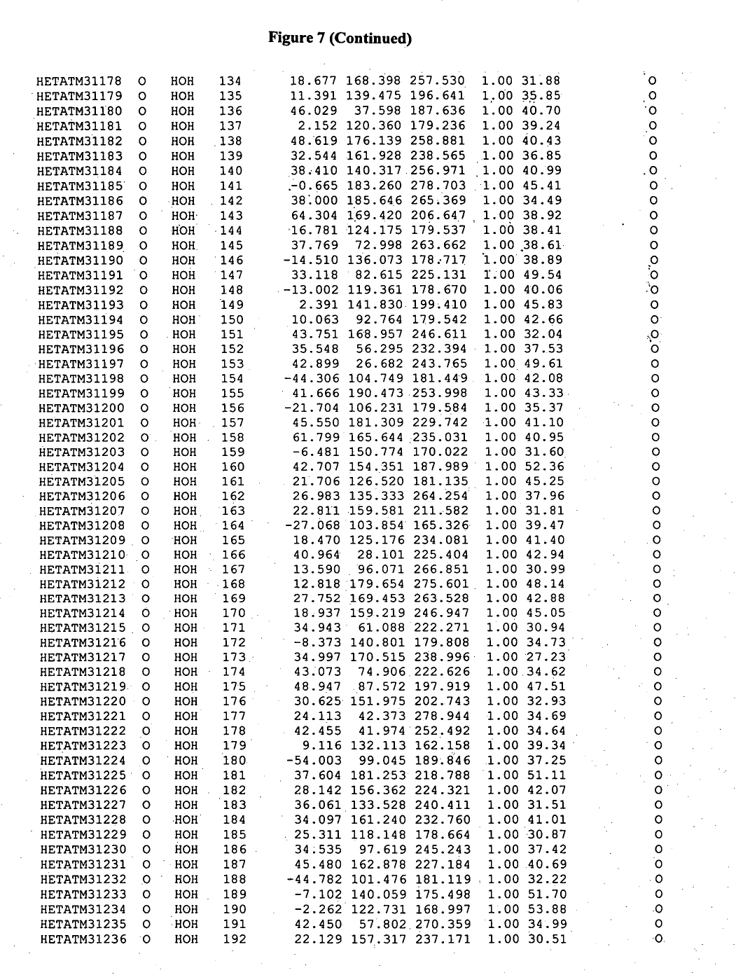

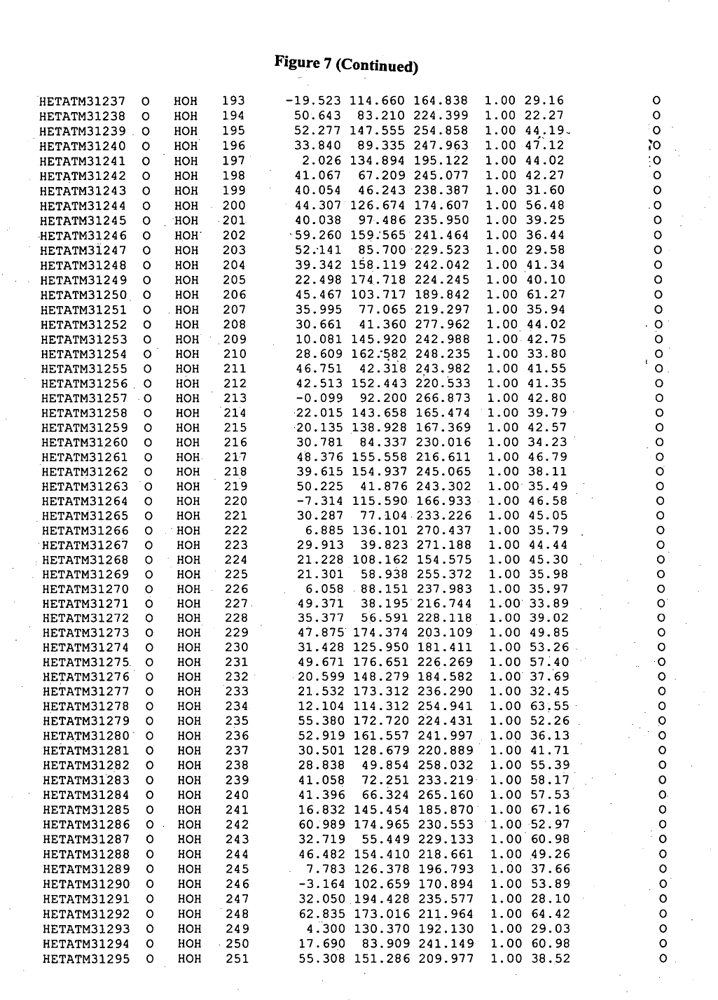

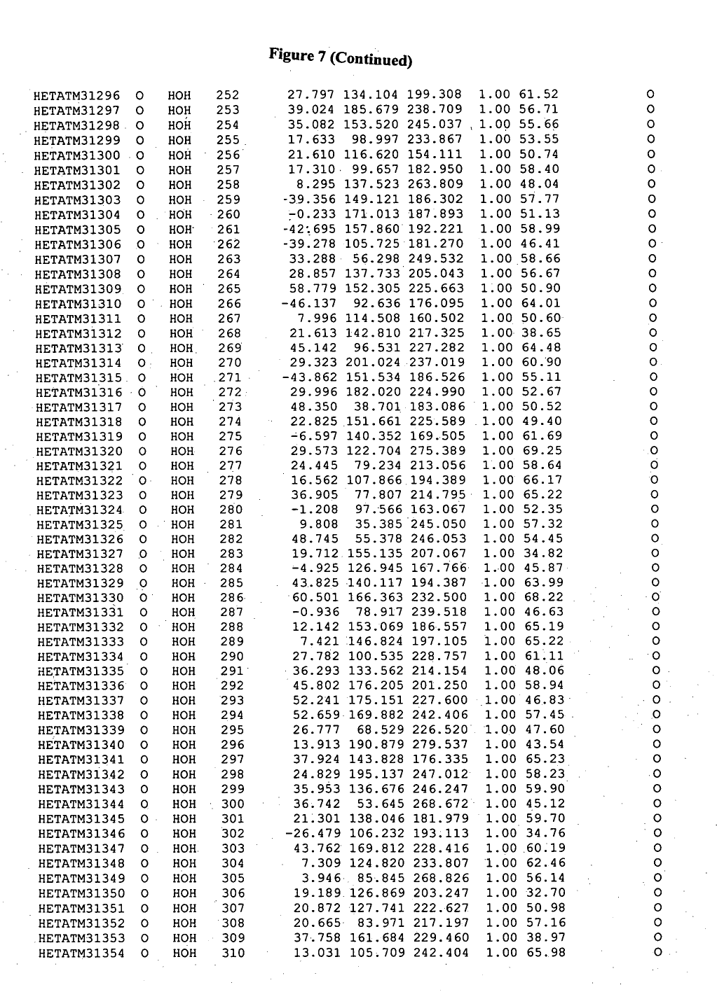

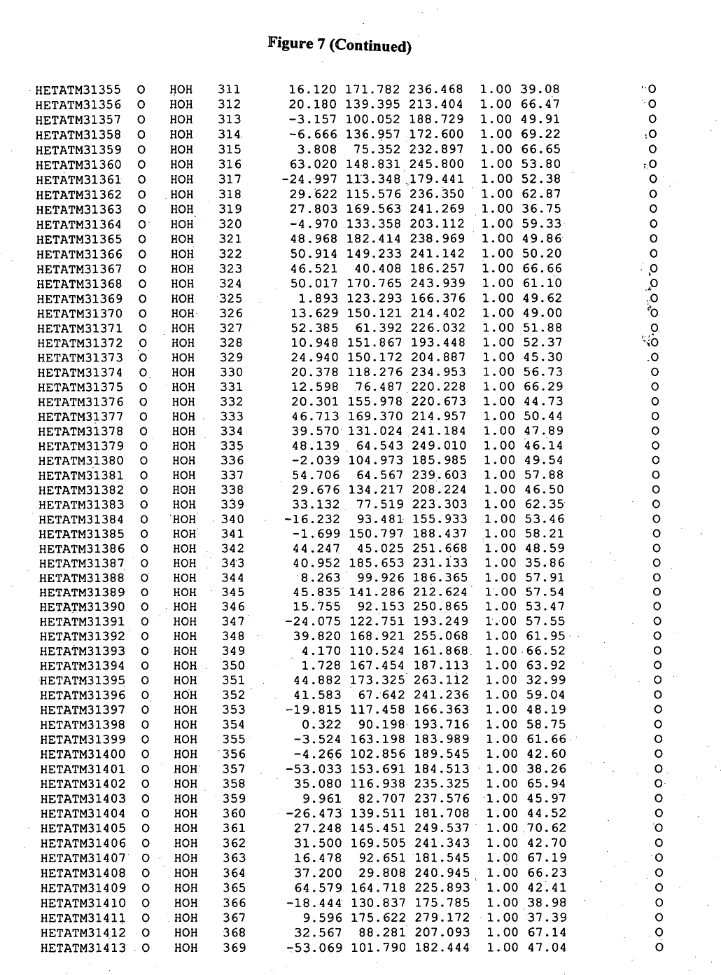

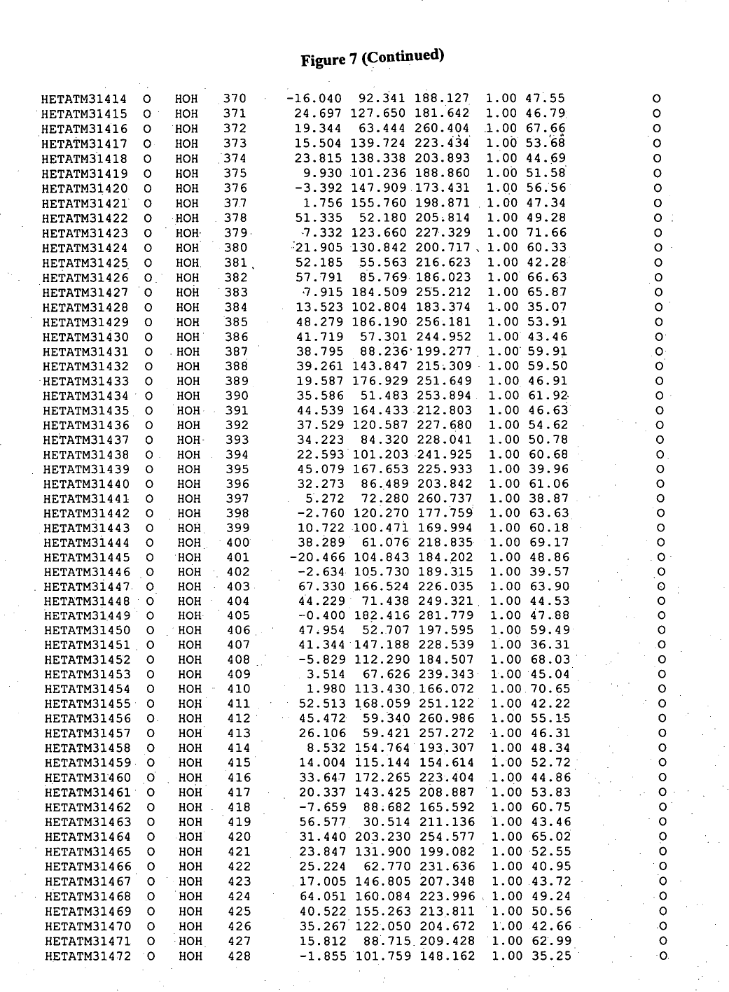

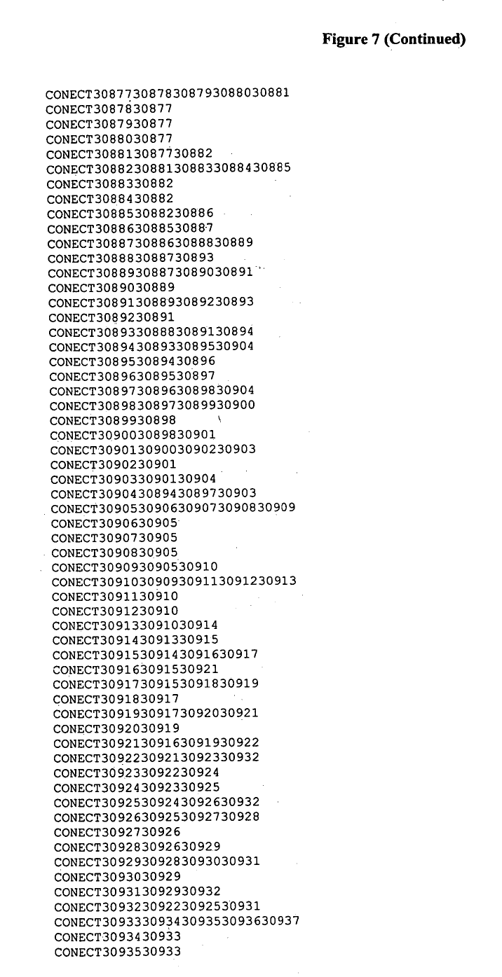

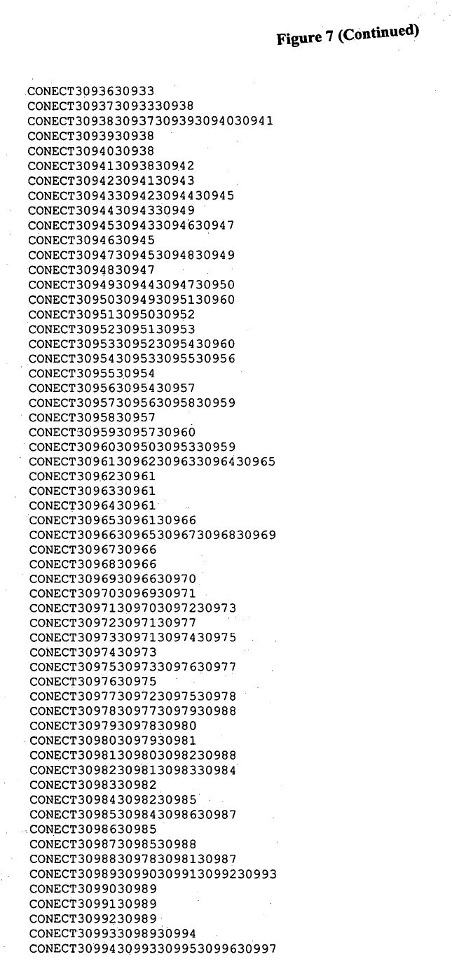

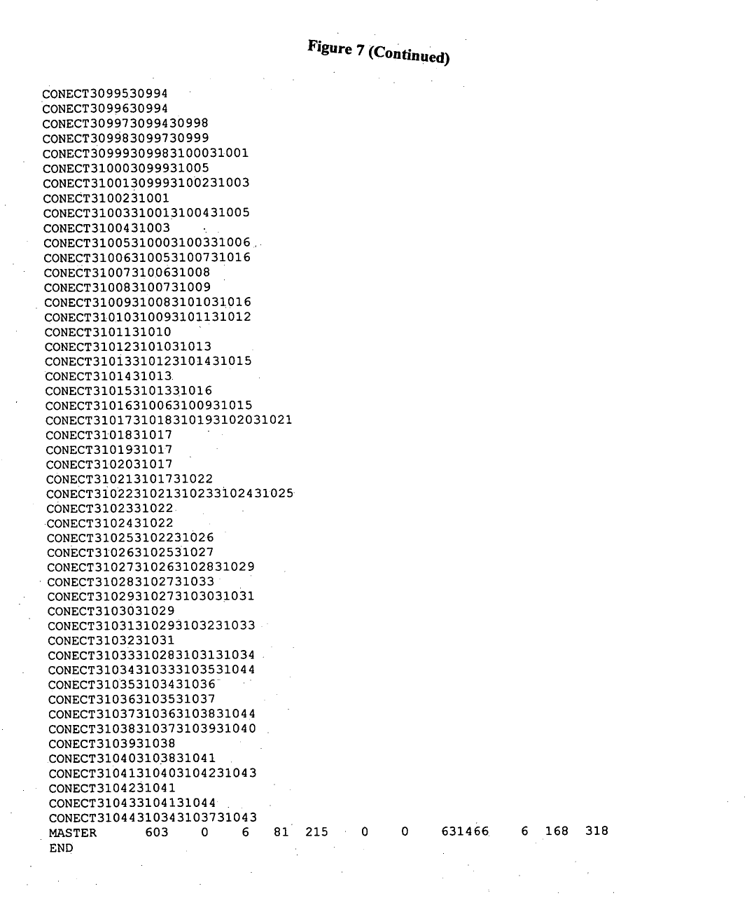

The inhibiting can be achieved with a compound which binds to one or more molecular surfaces of the tissue transglutaminase having a three dimensional crystal structure defined by the atomic coordinates set forth in FIG. 7.

In one embodiment of the present invention, the molecular surfaces of the tissue transglutaminase include atoms surrounding one or more of residues Lys-173, Phe-174, Arg-476, Arg-478, Val-479, Ser-482, Met-483, Arg-580, Leu-582, or Tyr-583.

The inhibiting of tissue transglutaminase can be carried out by administering an inhibitor of tissue transglutaminase orally, intradermally, intramuscularly, intraperitoneally, intravenously, subcutaneously, or intranasally. The inhibitor compounds of the present invention may be administered alone or with suitable pharmaceutical carriers, and can be in solid or liquid form, such as tablets, capsules, powders, solutions, suspensions, or emulsions.

The inhibitor compounds may be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these active compounds may be incorporated with excipients and used in the form of tablets, capsules, elixirs, suspensions, syrups, and the like. Such compositions and preparations should contain at least 0.1% of active compound. The percentage of the compound in these compositions may, of course, be varied and may conveniently be between about 2% to about 60% of the weight of the unit. The amount of active compound in such therapeutically useful compositions is such that a suitable dosage will be obtained.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar, or both. A syrup may contain, in addition to active ingredient, sucrose as a sweetening agent, methyl and propylparabens as preservatives, a dye, and flavoring such as cherry or orange flavor.

These active compounds may also be administered parenterally. Solutions or suspensions of these active compounds can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The inhibitor compounds may also be administered directly to the airways in the form of an aerosol. For use as aerosols, the compounds of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

Another aspect of the present invention relates to a method for identifying candidate compounds suitable for facilitating death of cancer cells in a subject. The method first involves contacting tissue transglutaminase with a compound. Those compounds which bind to the tissue transglutaminase are identified as candidate compounds suitable for facilitating death of cancer cells in a subject.

The present invention also relates to a method of producing a tissue transglutaminase crystal suitable for X-ray diffraction. The method first involves subjecting a solution of tissue transglutaminase under conditions effective to grow a crystal of tissue transglutaminase to a size suitable for X-ray diffraction. Then, a tissue transglutaminase crystal suitable for X-ray diffraction is obtained.

Current approaches to macromolecular crystallization are described in McPherson, Eur. J. Biochem., 189:1-23 (1990), which is hereby incorporated by reference in its entirety.

In one embodiment of the present invention, the tissue transglutaminase crystal has space group P2₁2₁2₁ and unit cell dimensions of approximately a=132.479 Å, b=168.797 Å, and c=238.568 Å such that the three dimensional structure of the crystallized tissue transglutaminase can be determined to a resolution of about 2.8 Å or better. Crystallization may be carried out by sitting drops using a vapor diffusion method.

In another embodiment, the present invention is a tissue transglutaminase crystal produced by the method of the present invention involving subjecting a solution of tissue transglutaminase under conditions effective to grow a crystal of tissue transglutaminase to a size suitable for X-ray diffraction, and obtaining a tissue transglutaminase crystal suitable for X-ray diffraction.

Another aspect of the present invention relates to a method for designing a compound suitable for facilitating death of cancer cells in a subject. The method first involves providing a three-dimensional structure of a crystallized tissue transglutaminase. Then, a compound having a three-dimensional structure which will bind to one or more molecular surfaces of the tissue transglutaminase is designed. The three dimensional structure of a crystallized tissue transglutaminase may be defined by the atomic coordinates set forth in FIG. 7. The molecular surfaces of the tissue transglutaminase can include atoms surrounding one or more of residues Lys-173, Phe-174, Arg-476, Arg-478, Val-479, Ser-482, Met-483, Arg-580, Leu-582, or Tyr-583. In facilitating cell death of cancer cells, the compounds designed by this method or pharmaceutical compositions containing such compounds (as well as a pharmaceutical carrier) are dosed and administered by the modes described above.

### EXAMPLES

The following examples are provided to illustrate embodiments of the present invention but are by no means intended to limit its scope.

### Example 1

### Expression and Purification of Human TG

TG was amplified with primers that introduced a XhoI site before the initial ATG codon and an EcORI site after the stop codon for the ORF of TG, by using a pGEX-MCS-HTG plasmid as a template (Lai et al., “C-Terminal Deletion of Human Tissue Transglutaminase Enhances Magnesium-Dependent GTP/ATPase Activity,” J. Biol. Chem., 271:31191-31195 (1996), which is hereby incorporated by reference in its entirety), and then subcloned into pET-28a vector (Novagen) to create TG with an N-terminal His₆ tag. Overnight cultures from colonies of Escherichia coli B121 (DE3) cells (Novagen) transformed with the expression vector were grown at 37° C. These were used to inoculate 1 liter of TP medium [2% bacto-tryptone/1.5% yeast extract/0.2% Na₂HPO₄/0.1% KH₂PO₄/0.8% NaCl/0.2% glucose (all wt/vol)]. The bacterial cells were then grown at 25° C. until the cell density reached an OD₆₀₀ reading of 0.6, at which point the temperature was reduced from 25° C. to 18° C. before induction with 1 μM isopropyl β-D-thiogalactoside (IPTG). The cultures were grown overnight at 18° C. and then the cells were harvested by centrifugation at 4° C.

All protein purification steps were performed on ice. Cell pellets from 4 liters of culture were lysed by sonication in 150 ml of lysis buffer (50 mM Na₂HPO₄, pH 7.5/400 mM NaCl/5 mM benzamidine/5 mM 2-mercaptoethanol) with 50 μM GTP, 50 μM ATP, and 50 μg/ml PMSF. Both GTP and ATP were included in the lysis buffer as possible stabilizing agents, as ATP as well as GTP has been suggested to bind to TG (Lai et al., “C-Terminal Deletion of Human Tissue Transglufaminase Enhances Magnesium-Dependent GTP/ATPase Activity,” J. Biol. Chem., 271:31191-31195 (1996), which is hereby incorporated by reference in its entirety). After sonication, Triton X-100 was added to a final concentration of 0.5% (vol/vol). Cell debris was removed by high-speed centrifugation and the supernatant was loaded onto a column containing 5 ml of Talon metal-affinity resins (CLONTECH). The column was washed with 150 ml of lysis buffer containing 20 μM GDP, and then further washed with 150 ml of 50 mM Hepes (pH 7.0)/150 mM NaCl/5 mM 2-mercaptoethanol/20 μM GDP/5 mM imidazole. The TG fusion protein was eluted with 50 mM Hepes (pH 7.0)/50 mM NaCl/5 mM 2-mercaptoethanol/20 μM GDP/160 mM imidazole. The eluted protein was loaded onto a MonoQ anion exchange column (Pharmacia Biotech) equilibrated with 50 mM Mes (pH 6.5)/50 mM NaCl/10% (vol/vol) glycerol/1 mM EDTA/5 mM DTT. After washing with the equilibration buffer, human TG was eluted by using a gradient of 150 mM to 450 mM NaCl in the same buffer. The fractions containing TG were pooled and concentrated to 2 ml by using UltraPrep filtration (Millipore, molecular weight cutoff=30,000), and then loaded onto a HiLoad 26/60 Superdex S-200 gel filtration column (Pharmacia Biotech) and eluted with 50 mM Hepes (pH 7.0)/100 mM NaCl/10% (vol/vol) glycerol/1 mM EDTA/5 mM DTT at 0.5 ml/min. Fractions containing TG were pooled and concentrated. Purity of TG was confirmed by SDS/PAGE, and its transamidation activity was assayed by the hydroxylamine method (Gross et al., “The Extended Active Site of Guinea Pig Liver Transglutaminase,” J. Biol. Chem., 250:4648-4655 (1975), which is hereby incorporated by reference in its entirety). The specific activity of TG is 1.0 μmol/min per mg, comparable to the value reported for guinea pig liver tissue TG (Singh et al., “Biochemical Effects of Retinoic Acid on GTP-Binding Protein/Transglutaminases in HeLa Cells,” J. Biol. Chem., 271:27292-27298 (1996), which is hereby incorporated by reference in its entirety). The guanine nucleotide-binding activity of purified TG was confirmed by photoaffinity labeling with [α-³²P]GTP (Singh et al., “Identification and Biochemical Characterization of an 80 Kilodalton GTP-Binding Transglutaminase,” Biochemistry, 34:15863-15871 (1995), which is hereby incorporated by reference in its entirety) and by monitoring guanine nucleotide-mediated inhibition of transamidation.

### Example 2

### Crystallization of Human TG

Crystals of TG were obtained by the sitting-drop vapor diffusion method, by mixing 20 mg/ml TG in 20 mM Hepes (pH 7.0)/1 mM EDTA/EGTA/5 mM DTT/20% (vol/vol) glycerol with equivalent amounts of precipitation solution containing 50 mM Mes (pH 6.6), 200 mM NaCl, 50 mM MgCl₂, 6-8% PEG 3350, and 5 mM DTT. Drops were set against 1 ml of precipitation solution plus 20% (vol/vol) glycerol at 4° C. Crystals usually appeared within a day and reached the full size of 0.4 mm×0.2 mm×0.1 mm in 2-3 days. The crystals belong to space group P2₁2₁2₁, with unit cell constants of a=136.478 Å, b=168.797 Å, and c=236.568 Å. After soaking in 50 mM Hepes (pH 7.0)/200 mM NaCl/5 mM MgCl₂/30% (vol/vol) glycerol/20% (wt/vol) PEG 3350 for about 2 weeks, a 2.8-Å data set was collected at the Advanced Photon Source (Chicago, Ill.) Beamline BioCAT 12C at 100 K. Reflection data were processed by using the program suite DENZO/XDISPLAY/SCALEPACK (Otwinowski et al., “Processing of X-Ray Diffraction Data Collected in Oscillation Mode,” Methods Enzymol., 276:307-326 (1997), which is hereby incorporated by reference in its entirety).

TABLE 1
Data Collection and Refinement Statistics

Space group: P2₁2₁2₁
Unit cell constants: 132.479 Å, 168.797 Å, 238.568 Å
Resolution: 51.8-2.8 Å
No. of reflections: Measured 1,141,553; Unique 124,870
Completeness (%): 94.6 (88.8) R_{merge} (%): 8.5 (47.0)
Refinement
R_{factor}, % 23.3; R_{free}, % 27.2
No. nonhydrogen atoms: protein, 25,932; GDP, 168; water, 428.
rms deviation from ideal bond length: 0.013 Å and bond angle: 1.7°

### Example 3

### Structural Analysis

The TG structure was solved by the molecular replacement method using the program MOLREP (Vagin et al., “MOLREP: An Automated Program for Molecular Replacement,” J. Appl. Cryst., 30:1022-1025 (1977), which is hereby incorporated by reference in its entirety) with the crystal structure of the human Factor XIIIa as a search model (PDB ID code: 1GGU; Yee et al., “Three-Dimensional Structure of a Transglutaminase: Human Blood Coagulation Factor XIII,” Proc. Natl. Acad. Sci. USA, 91:7296-7300 (1994), which is hereby incorporated by reference in its entirety). Six independent molecules were found in the asymmetric unit, with orientations consistent with the observed pseudo-622 symmetry seen in the self-rotation function search and locations consistent with the pseudo-B centering observed in the low resolution Patterson map. The amino acid residues of Factor XIIIa were replaced with the corresponding residues of TG. After rigid body refinement with the program CNS (Brunger et al., “Crystallography & NMR System: A New Software Suite for Macromolecular Structure Determination,” Acta Crystallogr. D, 54:905-921 (1998), which is hereby incorporated by reference in its entirety) the model was subjected to successive cycles of simulated annealing refinement using CNS and manual model building by using the program O (Jones et al., “Improved Methods For Building Protein Models In Electron-Density Maps And The Location Of Errors In These Models”, Acta Crystallogr. A, 47:110-119 (1991), which is hereby incorporated by reference in its entirety). Noncrystallographic symmetry (NCS) between the six independent molecules was used in electron density averaging and during early refinement. In the last stages of refinement, side-chain atoms of several residues involved in different crystal packing environments were released from NCS restraints. The final model was refined to a final R factor of 0.232 and a final R_{free} of 0.272 (Table 1) and has been deposited in the Protein Data Bank (PDB ID code: 1KV3).

### Example 4

### Structure of TG

The x-ray crystallographic model had six independent TG molecules in the asymmetric unit of a P2₁2₁2₁ unit cell, which were organized as three dimers to give an approximate P6₁22 space group. The overall structure of a TG dimer is shown in FIG. 1. Each monomer had four distinct domains: the amino-terminal β-sandwich domain (shown in green) consisting of residues Met-1 to Phe-139, the transamidation catalytic core domain (red) consisting of Ala-147 to Asn-460 (marked by the essential Cys-277 in ball-and-stick), and two carboxy-terminal β-barrel domains (the first in blue and the second in yellow), which include Gly-472 to Tyr-583, and Ile-591 to Ala-687, respectively. The general domain structure for TG was similar to that for Factor XIIIa (Yee et al., “Three-Dimensional Structure of a Transglutaminase: Human Blood Coagulation Factor XIII,” Proc. Natl. Acad. Sci. USA, 91:7296-7300 (1994), which is hereby incorporated by reference in its entirety). Dimerization buried 2,783 Å² of surface area (i.e., the sum of the surface buried by each monomer), with each monomer contributing the tip of the first β-barrel domain to the interface. The remainder of the dimerization interface consisted of the second β-barrel domain from one monomer, and the β-sandwich domain, the catalytic domain, and the second β-barrel domain from the other monomer.

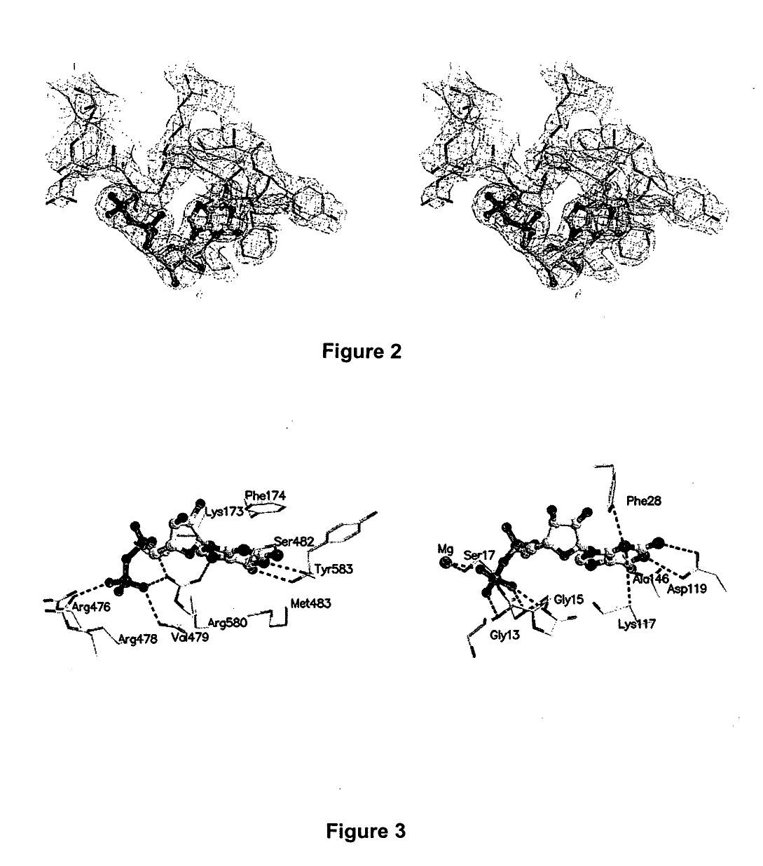

The guanine nucleotide-binding site was located in a cleft between the catalytic core and the first β-barrel domain (FIG. 1), close to the dimerization interface. The electron density unambiguously showed one GDP molecule bound to each of the six TG monomers within the asymmetric unit (FIG. 2). Finding bound GDP was unexpected because no GDP was present in the final purification or crystallization steps, and its presence testifies to both its tight binding and slow exchange. The majority of the residues contacting GDP came from the end of the first β-strand of the first β-barrel domain and the loop that connects it to the second β-strand, as well as from the last β-strand of the β-barrel domain (FIG. 1). The catalytic domain contained two residues interacting with the guanine base.

### Example 5

### Guanine Nucleotide-Binding Site of TG

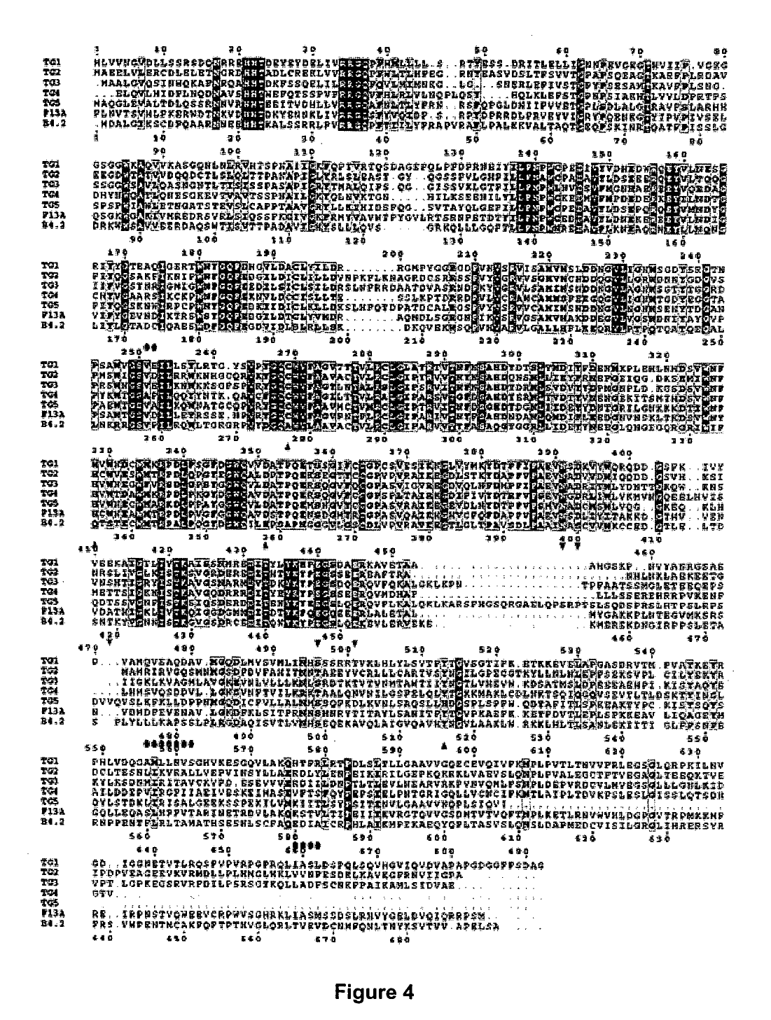

Overall, the architecture for the guanine nucleotide-binding site on TG differed markedly from the nucleotide-binding domain conserved among the α subunits of large heterotrimeric G proteins and small Ras-related G proteins, which have five helices surrounding a six-stranded β-sheet (Lambright et al., “Structural Determinants For Activation of the Alpha-Subunit of a Heterotrimeric G Protein”, Nature, 369:621-628 (1994), which is hereby incorporated by reference in its entirety). In heterotrimeric G proteins, the Gα subunits also contain a helical domain adjacent to the nucleotide-binding site. This helical domain probably enables the Gα subunits to bind guanine nucleotides with high affinity in the absence of Mg²⁺. Mg²⁺ is essential for the high-affinity binding of guanine nucleotides to Ras-related small G proteins, and guanine nucleotide exchange factors work by weakening the binding of Mg²⁺ (Sprang et al., “Invasion of the Nucleotide Snatchers: Structural Insights Into the Mechanism of G Protein GEFs”, Cell, 95:155-158 (1998), which is hereby incorporated by reference in its entirety). Like Ga, TG can bind GDP with high affinity in the absence of Mg²⁺ (Iismaa et al., “GTP Binding and Signaling by Gh/Transglutaminase II Involves Distinct Residues in a Unique GTP-binding Pocket,” J. Biol. Chem., 275:18259-18265 (2000), which is hereby incorporated by reference in its entirety), and the TG structure does not reveal any bound Mg²⁺. Although both large and small G proteins have serine and threonine residues that bind to the β- and γ-phosphates of the guanine nucleotide and participate in Mg²⁺ ion coordination (Lambright et al., “Structural Determinants For Activation of the Alpha-Subunit of a Heterotrimeric G Protein”, Nature, 369:621-628 (1994); Pai et al., “Structure of the Guanine-Nucleotide-Binding Domain of the Ha-ras Oncogene Product p21 in the Triphosphate Conformation”, Nature, 341:209-214 (1989), which are hereby incorporated by reference in their entirety), TG lacks amino acids with either hydroxyl or carboxyl side-chain moieties in the vicinity of the nucleotide phosphate groups. Several positively charged side chains surround the phosphate moieties of the bound GDP on TG (FIG. 3, Left). Arg-580 forms two ion pairs with the α- and β-phosphates, with the β-phosphate being positioned near the main chains of Arg-478 and Val-479 and forming a hydrogen bond with the nitrogen of Val-479 (FIG. 3, Left). Arg-478, Val-479, and Arg-580 are all conserved in tissue transglutaminases but not in other transglutaminases (FIG. 4).

There are a number of other interesting points of comparison between the guanine nucleotide-binding pocket of TG and the pocket for the traditional large and small G proteins. Of particular interest is the binding site for the guanine ring moiety. In both heterotrimeric large G proteins and small G proteins, the highly conserved NKXD motif plays an essential role in binding the guanine ring. The x-ray crystallographic structures of the Ga subunits of retinal transducin and the Gil protein (Lambright et al., “Structural Determinants For Activation of the Alpha-Subunit of a Heterotrimeric G-Protein,” Nature, 369:621-628 (1994); Noel et al., “The 2.2 Å Crystal-Structure of Transducin-α Complexed With GTP_{γ}S,” Nature, 366:654-663 (1993); Coleman et al., “Structures of Active Conformations of Gi alpha 1 and the Mechanism of GTP Hydrolysis,” Science, 265:1405-1412 (1994), which are hereby incorporated by reference in their entirety), as well as Ras (Pai et al., “Structure of the Guanine-Nucleotide-Binding Domain of the Ha-Ras Oncogene Product p21 in the Triphosphate Conformation,” Nature, 341:209-214 (1989), which is hereby incorporated by reference in its entirety), show that the asparagine residue of the NKXD sequence forms a hydrogen bond with the N7 atom of the guanine moiety, whereas the aspartic acid (Asp-19 of Ras in FIG. 3, Right) forms hydrogen bonds with the N1 and N2 atoms. The NKXD motif is not present in TG. Rather, a main-chain oxygen from Tyr-583 forms hydrogen bonds with the N1 and N2 atoms of the guanine base, and Ser-482 Oγ forms an additional hydrogen bond with N2 (FIG. 3, Left). In addition, O6 of the base forms a hydrogen bond with the main chain nitrogen of Tyr-583, a conserved residue in tissue transglutaminases.

In the TG structure, the guanine base sits in a hydrophobic pocket formed by the side chains from Phe-174, Val-479, Met-483, Leu-582, and Tyr-583 (FIG. 3, Left). The conserved phenylalanine residue might stabilize one side of the guanine ring through aromatic stacking interactions. There is no such corresponding phenylalanine in heterotrimeric G proteins. Both the Gα subunits of retinal transducin and the Gil protein use the methylene carbons of a second lysine residue within the signature NKXD motif (where X is the second lysine) to fulfill a similar function by making van der Waals contacts with one side of the guanine ring. However, it is worth noting that in Ras and other related small G proteins, a conserved phenylalanine (Phe-28 of Ras in FIG. 3, Right) approaches one side of the guanine ring at an approximately 90° angle (Pai et al., “Structure of the Guanine-Nucleotide-Binding Domain of the Ha-Ras Oncogene Product p21 in the Triphosphate Conformation,” Nature, 341:209-214 (1989), which is hereby incorporated by reference in its entirety), and has been suggested to participate in π-π stacking interactions. Mutation of this phenylalanine to leucine in the small G proteins Ras and Cdc42 yields constitutive GTP-GDP exchange activity, and in both cases gives rise to malignant transformation (Reinstein et al., “p21 With a Phenylalanine 28—Leucine Mutation Reacts Normally With the GTPase Activating Protein GAP but Nevertheless Has Transforming Properties,” J. Biol. Chem., 266:17700-17706 (1991); Lin et al., “Novel Cdc42Hs Mutant Induces Cellular Transformation,” Curr. Biol., 7:794-797 (1997), which are hereby incorporated by reference in their entirety). In TG, the opposite side of the guanine ring is in contact with the conserved residues Val-479 and Met-483, such that these residues together with Phe-174 sandwich the guanine moiety (FIG. 3, Left). This arrangement is not observed in Ras or other small G proteins, whereas in the Ga subunits of transducin and Gil, a conserved threonine residue within the carboxyl-terminal domain of the Gα subunits serves a function similar to that of Val-479 and Met-483 in TG (Lambright et al., “Structural Determinants For Activation of the Alpha-Subunit of a Heterotrimeric G-Protein,” Nature, 369:621-628 (1994), which is hereby incorporated by reference in its entirety).

In both Gα subunits and Ras-related small G proteins, a conserved glutamine is essential for GTP hydrolysis. Although TG has no such glutamine, it is capable of hydrolyzing GTP with a turnover number (≈1 mol of ³²Pᵢ released per min per mol of TG) similar to the intrinsic rates of GTP hydrolysis measured for Gα subunits, and the GTPase-activating protein (GAP)-catalyzed hydrolytic rates of small G proteins (Wittinghofer, “The Structure of Transducin G alpha T: More to View Than Just Ras,” Cell, 76:201-204 (1994), which is hereby incorporated by reference in its entirety). Given that the β-phosphate of the guanine nucleotide is pointed toward the Arg-478-Val-479 dipeptide (FIG. 3, Left), the γ-phosphate would need to rotate around the β-phosphate-O3′ bond to avoid clashing with the side chains of these amino acids. This rotation would bring the γ-phosphate into the vicinity of the positively charged side chains of Lys-173 and Arg-476. A plausible mechanism for TG-catalyzed GTP hydrolysis may involve a water hydrogen bonded to either the side chain of Lys-173 or Arg-476 as the nucleophilic attacking group. The positive charges of Lys-173, Arg-476, and Arg-478 would likely help orient the γ-phosphate group as well as stabilize the negative charges that develop on the y-phosphate group during hydrolysis. Mutations of Lys-173 significantly impair GTP hydrolysis, which is consistent with this proposal (Iismaa et al., “GTP Binding and Signaling by Gₕ/Transglutaminase II Involves Distinct Residues in a Unique GTP-Binding Pocket,” J. Biol. Chem., 275:18259-18265 (2000), which is hereby incorporated by reference in its entirety). In this mechanistic formulation, either Arg-476 or Arg-478 could serve as the “arginine finger,” which has been shown to be essential for stabilizing the transition states for GTP hydrolysis by both large and small G proteins (Scheffzek et al., “The Ras-RasGAP Complex: Structural Basis for GTPase Activation and its Loss in Oncogenic Ras Mutants,” Science, 277:333-338 (1997), which is hereby incorporated by reference in its entirety). Lys-173, Arg-476, and Arg-478 are conserved or conservatively substituted (Lys-Arg) in tissue transglutaminases.

Studies have shown that among the transglutaminase family only TG (TG2 in FIG. 4) can bind and use guanine nucleotides to regulate transamidation. Indeed, multiple sequence alignments of different human transglutaminases show that the amino acid residues involved in GDP binding in TG are not remotely conserved (see the blue dots in FIG. 4, which are placed below the residues essential for GTP-binding to TG2). For example, Phe-174 is replaced by aspartic acid in the Factor XIIIa sequence. On the other hand, the sequences of all TGs known to bind guanine nucleotides are highly conserved, including those residues that form the nucleotide-binding site.

### Example 6

### Regulation of Transamidation Activity

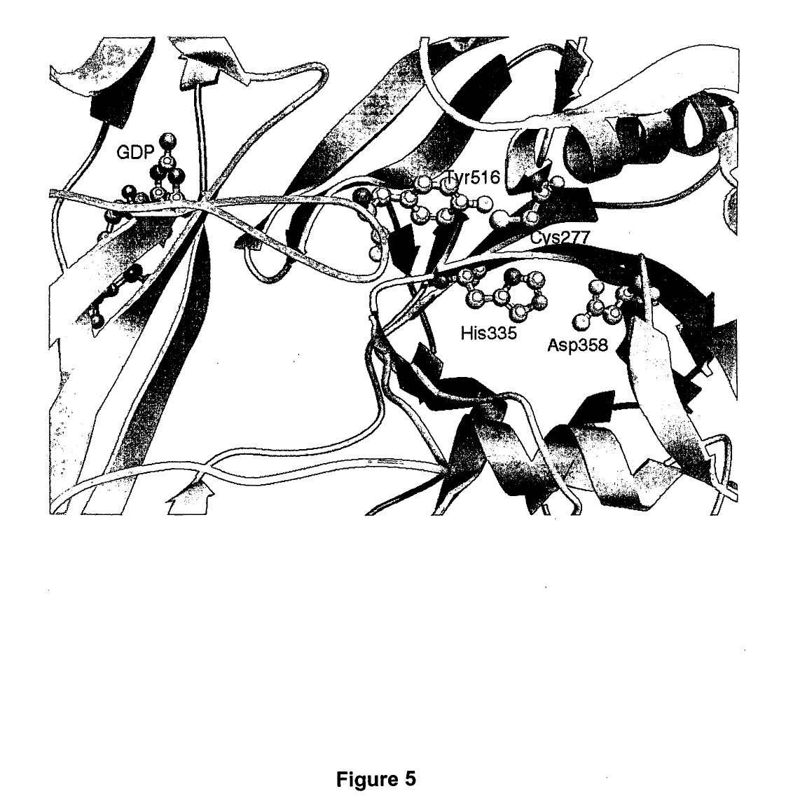

The TG structure also provided clues regarding the regulation of its enzymatic transamidation activity. It has been well established that Cys-277 is the essential nucleophile for transamidation (Yee et al., “Three-Dimensional Structure of a Transglutaminase: Human Blood Coagulation Factor XIH,” Proc. Natl. Acad. Sci. USA, 91:7296-7300 (1994), which is hereby incorporated by reference in its entirety). In the TG structure, Cys-277 is located in the middle of a groove within the catalytic domain (FIG. 1) and participates in a catalytic triad, Cys-277-His-335-Asp-358 (FIG. 5), similar to what has been reported for Factor XIIIa (Yee et al., “Three-Dimensional Structure of a Transglutaminase: Human Blood Coagulation Factor XIII,” Proc. Natl. Acad. Sci. USA, 91:7296-7300 (1994), which is hereby incorporated by reference in its entirety). These three catalytic residues are conserved in all members of the transglutaminase family (FIG. 4). In the GDP-bound form of TG, access to the transamidation active site is blocked by a loop connecting the third and fourth β-strands, as well as by a loop connecting the fifth and sixth β-strands of the first β-barrel domain (the loops are shown in blue in FIG. 5). Tyr-516, which is conserved in TGs and located in the first loop, forms a hydrogen bond with Cys-277 (FIG. 5). Transamidation activity requires an accessible Cys-277, and Tyr-516 with its associated loop from the first β-barrel domain must move to make the active site accessible to substrates. The GDP molecule engages both the first and last β-strands of the first β-barrel domain, which should maintain the inactive state by stabilizing the loops that block access to the catalytic domain. This observation would likely account for the observations that guanine nucleotide binding inhibits transamidation activity (Achyuthan et al., “Identification of a Guanosine Triphosphate-Binding Site on Guinea Pig Liver Transglutaminase. Role of GTP and Calcium Ions in Modulating Activity,” J. Biol. Chem., 262:1901-1906 (1987); Lai et al., “C-terminal Deletion of Human Tissue Transglutaminase Enhances Magnesium-dependent GTP/ATPase Activity,” J. Biol. Chem., 271:31191-31195 (1996), which are hereby incorporated by reference in their entirety).

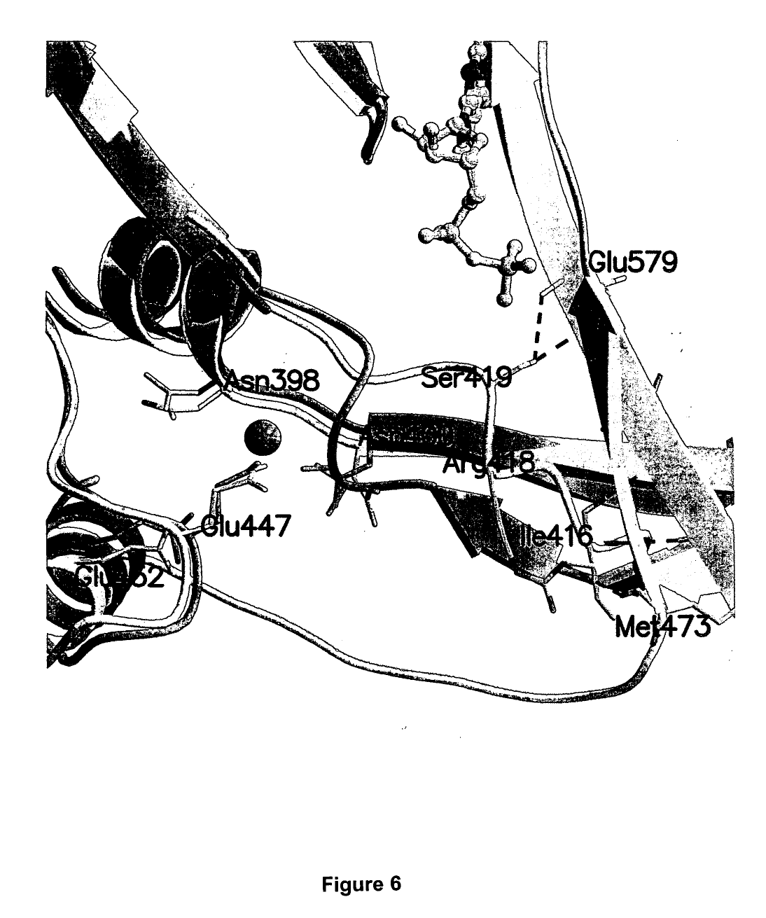

Calcium ions exert an activating signal for transamidation (Achyuthan et al., “Identification of a Guanosine Triphosphate-Binding Site on Guinea Pig Liver Transglutaminase. Role of GTP and Calcium Ions in Modulating Activity,” J. Biol. Chem., 262:1901-1906 (1987); Singh et al., “Identification and Biochemical Characterization of an 80 Kilodalton GTP-Binding Transglutaminase from Rabbit Liver Nuclei,” Biochemistry, 34:15863-15871 (1995), which are hereby incorporated by reference in their entirety). The structures for Factor XIIIa complexed to calcium, strontium, and ytterbium show that a major Ca²⁺-binding site is formed by the side chains of the conserved Asn-436, Asp-438, Glu-485, and Glu-490, and by the main chain oxygen of Ala-457 (Fox et al., “Identification of the Calcium Binding Site and a Novel Ytterbium Site in Blood Coagulation Factor XIII by X-ray Crystallography,” J. Biol. Chem., 274:4917-4923 (1999), which is hereby incorporated by reference in its entirety; also see FIG. 6). The putative Ca²⁺-binding site on TG is located near the end of the loop that connects the catalytic transamidation domain to the first α-barrel domain. Unlike the case for Factor XIIIa, this site is distorted in TG, with the largest difference occurring in the vicinity of Ser-419 (equivalent to Ala-457 in Factor XIIIa, FIG. 6). In TG, peptide IIe-416-Ser-419 forms a β-strand antiparallel with peptide Leu-577-Glu-579. Apparently these hydrogen bonds involved in β-sheet formation can support the first β-barrel domain and further stabilize the nucleotide-binding site. Calcium binding, by altering the position of the Ile-416-Ser-419 peptide, would eliminate these stabilizing effects and could thereby weaken nucleotide binding, as has been observed experimentally (Achyuthan et al., “Identification of a Guanosine Triphosphate-Binding Site on Guinea Pig Liver Transglutaminase. Role of GTP and Calcium Ions in Modulating Activity,” J. Biol. Chem., 262:1901-1906 (1987), which is hereby incorporated by reference in its entirety). In Factor XIIIa, the equivalent peptide, Asn-454-Ala-457, forms an antiparallel β-strand with Asp-458-Tyr-441, which stabilizes calcium binding. Glutamic acid residues 447 and 452 may also undergo Ca²⁺-induced conformational changes that would further impact the nucleotide site and weaken nucleotide binding. In an apoptotic cell, falling nucleotide levels and increasing Ca²⁺ levels would activate TG's transamidation activity.

Subtle differences in the conformations induced by GTP versus GDP could explain some reports that have shown differences in the extent of transamidation activity measured for the two nucleotide states of TG (Achyuthan et al., “Identification of a Guanosine Triphosphate-Binding Site on Guinea Pig Liver Transglutaminase. Role of GTP and Calcium Ions in Modulating Activity,” J. Biol. Chem., 262:1901-1906 (1987); Singh et al., “Identification and Biochemical Characterization of an 80 Kilodalton GTP-Binding Transglutaminase from Rabbit Liver Nuclei,” Biochemistry, 34:15863-15871 (1995), which are hereby incorporated by reference in their entirety). The ability of TG to undergo a GTP-binding/GTPase cycle that is conformationally coupled to its enzymatic transamidation activity potentially offers an interesting example of a G protein that has a “built-in” effector enzyme activity, and perhaps underlies the unique architecture of its guanine nucleotide-binding site. It also raises the likelihood that distinct types of extracellular stimuli (e.g., retinoic acid; Singh et al., “Biochemical Effects of Retinoic Acid on GTP-Binding Protein/Transglutaminases in HeLa Cells,” J. Biol. Chem., 271:27292-27298 (1996), which is hereby incorporated by reference in its entirety) and as yet undescribed types of regulatory proteins will be involved in the regulation of the GTP-binding and GTP-hydrolytic activities of TG, relative to those that have been reported for the more traditional large and small G proteins.

### Example 7

### Enhanced TG Activity in Breast Tumor Cell Lines

Tissue transglutaminase (TG) is a multifunctional protein with an enzymatic transamidation activity that covalently links proteins to other proteins or polyamines and a GTP-binding/GTP-hydrolysis cycle similar to other classical G-proteins. The transamidation activity of TG is highly regulated as underscored by the following two points. First, only a few proteins have been shown to serve as substrates for TG transamidation in vivo, suggesting that substrate specificity contributes to the overall management of this enzymatic reaction. Second, the cross-linking of substrates by TG occurs only after the protein has proceeded through multiple regulatory events resulting in a TG species capable of enzymatic activity. One such event involves the ability of TG to bind and hydrolyze GTP. In vitro studies have demonstrated that GTP-bound, but not GDP-bound TG inhibited enzymatic activity. Other work has shown that a point mutation in the GTP-binding domain of TG that disrupts GTP-binding also inhibited transamidation. These findings indicate that although GTP-bound TG limits transamidation activity the ability of TG to bind and then hydrolyze GTP is essential to induce enzymatic activity.

Many cell types express TG at low levels and increases in TG expression and enzymatic activity typically occur following exposure to differentiation agents and apoptotic-inducing stimuli. For example, retinoic acid (RA), which has received attention as a cancer therapy due to its growth inhibitory activity, is a consistent inducer of TG expression and activation. RA-mediated TG enzymatic activity was shown to be essential for neurite extension in SHY5Y5 cells, while blocking RA-stimulated TG expression in SK-N-BE or U937 cells rescued these cells from RA-induced apoptosis. Moreover, expression of an oncogenic form of Ras in fibroblasts suppressed RA-mediated TG expression, implying that retinoid stimulated TG expression was inconsistent with the Ras-transformed phenotype. As a result of these and similar findings, such as the proposed use of TG hypo-expression as a biomarker for prostate cancer, it has become generally accepted that TG promotes cellular processes that limit cell number. However, recent evidence suggests that TG may not be detrimental to the growth of all cell types. For instance, exogenous expression of TG in mouse fibroblasts not only provided a protective effect from serum deprivation-mediated apoptosis, but also did not compromise the proliferative capacity of these cells. Furthermore, aberrant TG expression has been noted in some human brain and breast tumors, but the relevance of TG overexpression to the progression of these cancers is unknown.

New insights regarding the regulation and function of TG have come from studies investigating the affect of signaling events on TG expression and activation. The Ras-extracellular signal-regulated kinase (ERK) pathway and the phosphoinositide 3-kinase (PI3K)-AKT pathway are signal transduction cascades that have received attention for their ability to mediate growth factor-stimulated processes such as inducing cell cycle progression and promoting cell survival. In addition to growth factors, like epidermal growth factor (EGF), retinoids can stimulate the activation of these pathways in certain cell types; therefore, it was investigated whether the ability of RA and EGF to activate the Ras-ERK and PI3 kinase-AKT pathways influenced TG expression and activation in the mouse fibroblast cell line NIH3T3. RA-stimulation resulted in the activation of PI3 kinase, which is required for the induction of TG expression and GTP-binding ability. In contrast, EGF-stimulation antagonized the ability of RA to induce TG expression by activating the Ras-ERK pathway. The use of mouse fibroblasts (NIH3T3 cells) for these studies proved valuable in revealing complex signaling profiles that govern the activation of TG, yet whether the same signaling events regulate TG expression and activation in other cell types remains to be established. The effects of EGF on RA-stimulated TGase expression and activation in the human breast cancer cell line SKBR3 were also examined. In contrast to NIH3T3 cells, EGF did not inhibit RA-induced TG expression and activation in the breast cancer cell line. Even more intriguing was that EGF-stimulation alone augmented TG expression and activation more efficiently than RA. Both RA and EGF-induced TG expression required PI3 kinase activity, implicating PI3 kinase as a common regulator of growth factor and retinoid mediated TG expression in SKBR3 cells. Inhibiting EGF-stimulated TG activity resulted in a nearly complete loss of EGF-mediated protection from doxyrubicin-induced apoptosis. These findings suggest for the first time that EGF-stimulated TG activity contributes to the oncogenic potential of SKBR3 cells. The methods used in the experiments as well as the results are described in further detail in Examples 8-17.

### Example 8

### Materials Used in Examples 9-17

LY294002 and doxorubicin were obtained from Calbiochem, and EGF was from Invitrogen. RA and monodansylcadaverine (MDC) were purchased from Sigma, while the 5-(biotin-amido) pentylamine was from Pierce. The TG antibody was obtained from Neomarkers, the actin antibody was from Sigma, and the phospho-ERK and AKT antibodies and cleaved caspase-3 antibody were from Cell Signaling. The EGF receptor and the phospho-EGF receptor antibodies were purchased from Transduction Labs, and the HA and Myc antibodies were from Covance. [α-³²P]GTP was purchased from Perkin-Elmer Life Sciences. All additional materials were obtained from Fisher unless stated otherwise.

### Example 9

### Cell Culture

NIH3T3 cells were grown in Dulbecco's modified Eagle's medium containing 10% calf serum and 100 units/ml penicillin. SKBR3 and MDAMB231 cells were grown in RPMI 1640 medium containing 10% fetal bovine serum and 100 units/ml penicillin. The cell lines were maintained in a humidified atmosphere with 5% CO₂ at 37° C. For the various treatments described, the cells were grown to near confluence in medium containing 10% serum, and then medium containing 1% serum or no serum with 5 μM RA, and/or 100 ng/ml EGF, +/−7 μM LY294002, and +/−0.2 μM doxorubicin were added for the appropriate amount of time. Cells were rinsed with phosphate-buffered saline (PBS) and then lysed with cell lysis buffer (10 mM Na₂HPO₄, 150 mM NaCl, 1% Triton X-100, 0.5% sodium deoxycholate, 0.1% SDS, 0.004% NaF, 1 mM NaVO₄, 25 mM β-glycerophosphoric acid, 100 μg/ml phenylmethanesulfonyl fluoride, and 1 μg/ml each aprotinin and leupeptin, pH 7.35). The lysates were clarified by centrifugation at 12,000×g for 10 minutes at 4° C. Protein concentrations were determined using the Bio-Rad DC protein assay.

### Example 10

### Western Blot Analysis

Total cell lysates from each sample were combined with Laemmli sample buffer, boiled, and subjected to SDS-polyacrylamide gel electrophoresis (PAGE). The proteins were transferred to nitrocellulose filters and blocked with TBST (20 mM Tris, 137 mM NaCl, pH 7.4, and 0.02% Tween 20) containing 5% nonfat dry milk. The filters were incubated with the various primary antibodies diluted in TBST overnight at 4° C., then washed 3 times with TBST. To detect the primary antibodies, anti-mouse or rabbit conjugated to horseradish peroxidase (Amersham Corporation), diluted 1:5000 in TBST, was incubated with the filters for 1 hour, followed by 3 washes with TBST. The protein bands were visualized on X-ray film after exposing the filters to chemiluminescence reagent (ECL, Amersham Corporation).

### Example 11

### Photoaffinity Labeling of TG

Photoaffinity labeling of TG was performed by incubating whole cell lysates with 5 μCi of [α-³²P]GTP in 50 mM Tris-HCl, pH 7.4, 2 mM EGTA, 1 mM DTT, 20% (w/v) glycerol, 100 mM NaCl, and 500 μM AMP-PNP for 10 minutes at room temperature. The samples were placed in an ice bath and irradiated with UV light (254 nm) for 15 minutes, mixed with 5× Laemmli sample buffer, and boiled. SDS-PAGE was performed, followed by transfer to nitrocellulose filters, and exposure on X-ray film.

### Example 12

### Transamidation Assay

TG transamidation assays were performed as previously described with slight modifications. 15 μg of whole cell extracts were incubated in a buffer containing 2 mM 5-(biotin-amido) pentylamine, 40 mM CaCl₂, and 40 mM DTT for 20 minutes. The reaction was stopped by the addition of Laemmli sample buffer followed by boiling. The reactions were then resolved on a 4-20% gradient SDS-polyacrylamide gel, transferred to nitrocellulose filters, and then blocked overnight with BBST (100 mM boric acid, 20 mM sodium borate, 0.01% SDS, 0.01% Tween, and 80 mM NaCl) containing 5% bovine serum albumin (BSA). The filters were incubated with HPR-conjugated streptavidin diluted 1:3000 in TBST for 2 hours at room temperature, and then washed 5 times with BBST. The proteins that incorporated 5-(biotin-amido) pentylamine were visualized on X-ray film after exposing the filters to chemiluminescence reagent (ECL, Amersham Corporation).

### Example 13

### Nuclear Condensation or Blebbing Assay

Cells were seeded in 6-well dishes and grown in complete medium for 2 days. The cells were then incubated in medium containing 1% serum +/−5 μM RA, +/−100 ng/ml EGF, and +/−10 μM MDC for 2 days. The cultures were then incubated with fresh medium containing RA, EGF, and MDC and +/−0.2 μM doxorubicin for an additional 1.5 days and were fixed and stained with 4,6-diamidino-2-phenylindole (2 μg/ml) for viewing by fluorescence microscopy. Apoptotic cells were identified by condensed nuclei and/or blebbing.

### Example 14

### Cell Growth Assay

Cells were seeded at 1×10⁵ cells/well and grown in medium containing 0.5% serum +/−5 μM RA and +/−100 ng/ml EGF. Every 2 days the medium was changed. At 0, 2, 4, and 6 days of treatment, the cells were collected and counted.

### Example 15

### EGF Stimulates TG Expression and Activation in SKBR3 Cells

Previous work on the regulation of TG activity in NIH3T3 fibroblasts showed that RA-induced TG expression and activation could be blocked by EGF stimulation. To expand upon these findings, it was investigated whether the same interplay that exists between EGF and RA on TG expression and activation in NIH3T3 cells also occurs in other cell lineages. Since it has been well established that activation of the EGF receptor promotes cell growth and survival and overexpression of the EGF receptor has been implicated in the progression of several types of human cancers, conducting these experiments in tumor cells that overexpress the EGF receptor had the most potential to be interesting. To this end, the effects of EGF on RA-induced TG expression in the human breast tumor cell line SKBR3 were studied. These cancer cells were well suited for this study because they were not only previously shown to express the EGF receptor at relatively high levels, but they were also found to be responsive to RA stimulation as indicated by changes in gene transcription following exposure to the retinoid.

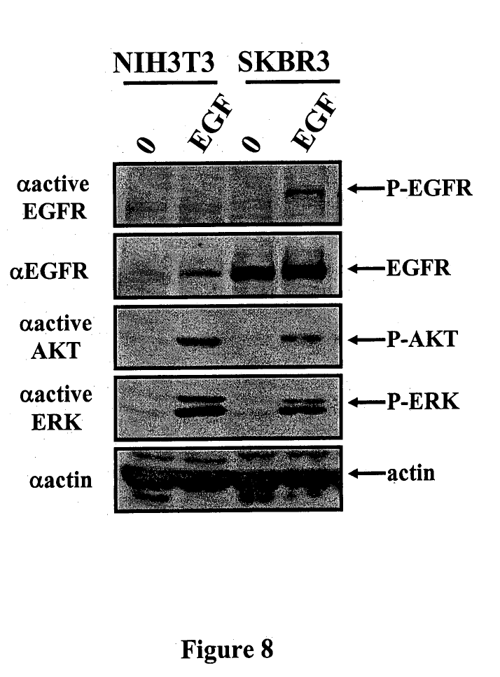

Initially, it was confirmed that the SKBR3 cells overexpressed the EGF receptor and that the receptor was functional. FIG. 8 (α-EGFR) shows that both NIH3T3 and SKBR3 cells express detectable amounts of the EGF receptor as determined by Western blot analysis. Consistent with reports showing that SKBR3 cells overexpress the EGF receptor, the level of EGF receptor expression in SKBR3 cells was considerably higher than in NIH3T3 cells. Then, the ability of EGF to activate its receptor and stimulate intercellular signaling events in the cancer cell line was assayed. This was accomplished through the use of antibodies that specifically recognize the activated form of the EGF receptor or AKT and ERK, two signaling molecules consistently activated in an EGF receptor-dependent manner. Treatment of SKBR3 cells with EGF for 5 minutes resulted in EGF receptor activation (FIG. 8; α-active EGFR). Coinciding with this activation were increases in the activities of both AKT (FIG. 8; α-active AKT) and ERK (FIG. 8; α-active ERK), indicating the EGF receptor was capable of signaling in these cells.

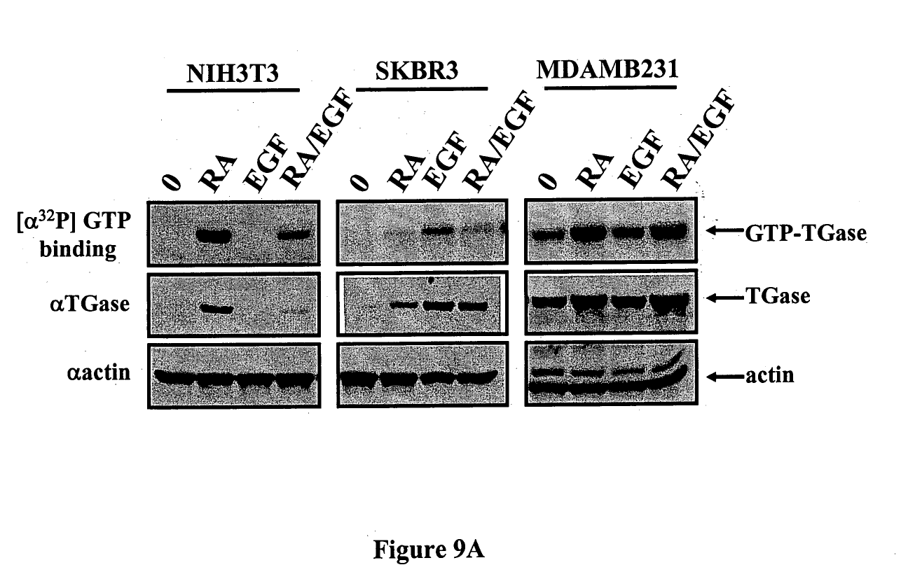

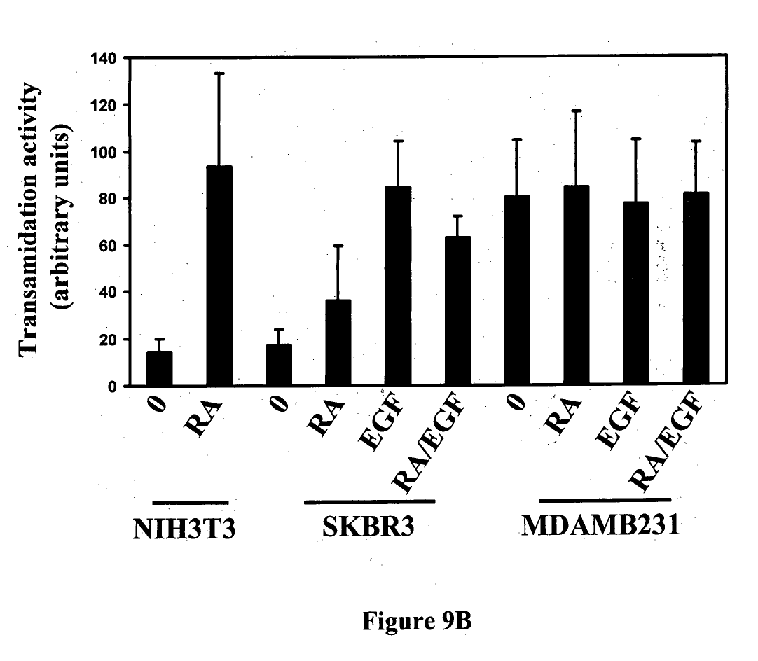

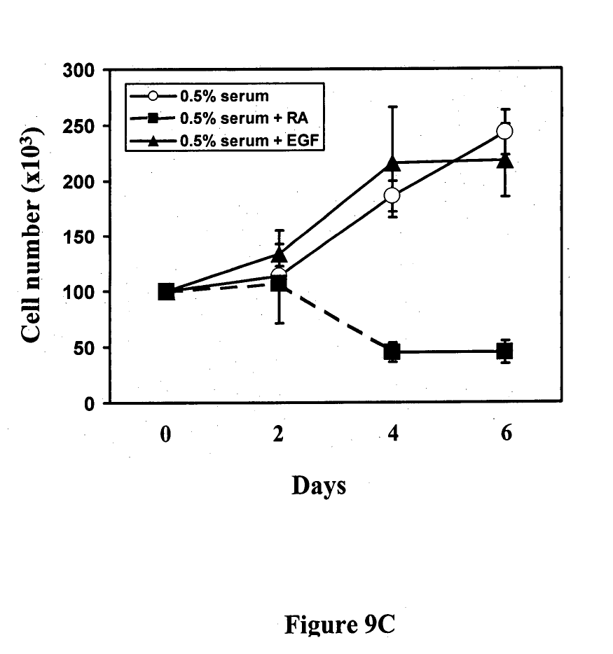

Then, it was examined whether RA could up-regulate TG expression and activation in SKBR3 cells. As shown previously, NIH3T3 cells stimulated with RA for 2 days induced TG expression (FIG. 9A; α-TGase) and GTP-binding activity (FIG. 9A; [α-³²P] GTP binding) as measured by the incorporation of [α-³²P] GTP into TG. Parallel experiments conducted on SKBR3 cells showed that RA treatment significantly increased TG expression over the nearly undetectable amounts of TG protein in unstimulated cells (FIG. 9A; α-TGase). Corresponding with this increase in TG expression was an increase in the GTP-binding activity of the molecule (FIG. 9A; [α-³²P] GTP binding), demonstrating that RA affects TG expression and GTP-binding ability similarly in both NIH3T3 and SKBR3 cells.

The above data establishes that SKBR3 cells express relatively high levels of functional EGF receptor and that TG expression and activation can be augmented by RA stimulation of these cells. Then, the effect of EGF on RA-mediated TG expression in a cancer cell line that over-expresses the EGF receptor was examined. As seen in FIG. 9A (α-TGase), exposure of SKBR3 cells to EGF did not inhibit RA-induced TGase expression as in NIH3T3 cells. Even more interesting than EGF not compromising retinoid-mediated TG expression in SKBR3 cells was the fact that growth factor-stimulation alone up-regulated TG expression. The EGF-induced TG was completely active as indicated by increased GTP-binding (FIG. 9A; [α-³²P] GTP binding) and transamidation activity (FIG. 9B), as readout by the incorporation of 5-(biotin-amido) pentylamine into proteins from cell extracts. Notably, although both RA and EGF induced TG expression and activation (including GTP-binding and transamidation activity) in SKBR3 cells, the growth factor was more effective than RA at stimulating the GTP-binding and enzymatic activity of TG. Since EGF, a known mitogen and survival factor, strongly induced the expression and activation of TG, a protein that has most often been linked to apoptosis or cell differentiation, it was next examined how EGF stimulation would affect the growth rate of SKBR3 cells. FIG. 9C shows that SKBR3 cells were able to proliferate in low serum medium, a characteristic often associated with cancer cells. While RA treatment severely diminished the growth of the cancer cell line, EGF treatment did not. The fact that EGF stimulation did not impede the growth of SKBR3 cells, despite strongly activating TG, indicates that the induction of TG expression and activation is not necessarily detrimental to the growth rate of SKBR3 cells.

The unique regulation of TG expression and activation observed in SKBR3 cells compared to NIH3T3 cells was not limited to this cell line, as another human breast cancer cell line, MDAMB231, was also found to exhibit a profile of TG expression and activation that was different from NIH3T3 cells. TG protein could be detected in MDAMB231 cells prior to RA or EGF treatment (FIG. 9A; α-TGase). This TG species displayed GTP-binding (FIG. 9A; [α-³²P] GTP binding) and enzymatic transamidation activity (FIG. 9B) that was comparable to the TG activity seen in SKBR3 cells treated with EGF. Growth factor stimulation did not alter the constitutive level of TG expression or activation in MDAMB231 cells, indicating that EGF-mediated signaling was not a negative regulator of TG expression in these breast cancer cells as well as in SKBR3 cells. The findings associating constitutive or EGF-induced TG expression and activation with MDAMB231 and SKBR3 cells suggest that, in addition to being stimulated by factors that induce cell differentiation or apoptosis, TG appears to also be positively managed by stimuli typically associated with cell growth and survival in certain human breast cancer cell lines.

### Example 16

### PI3 Kinase Activity is Essential for EGF-Induced TG Expression in SKBR3 Cells

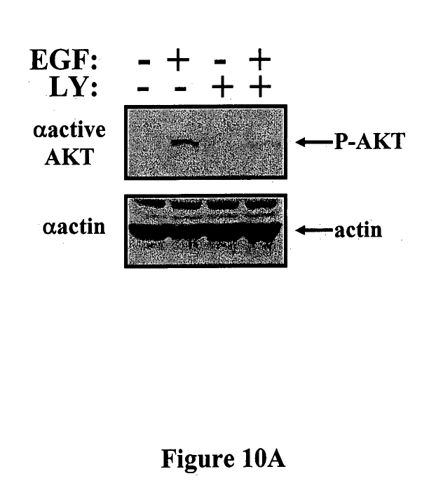

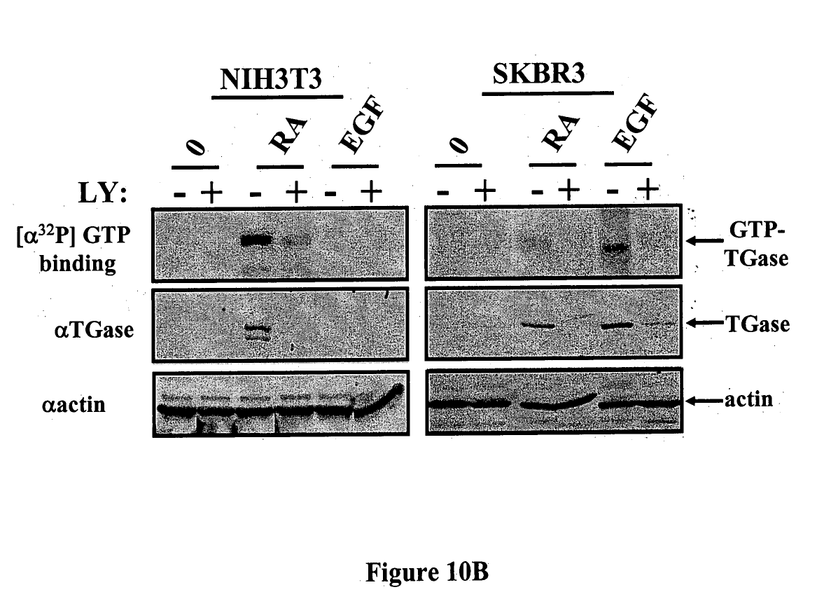

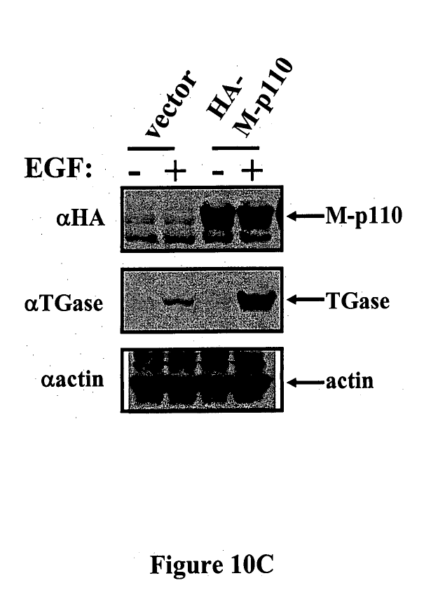

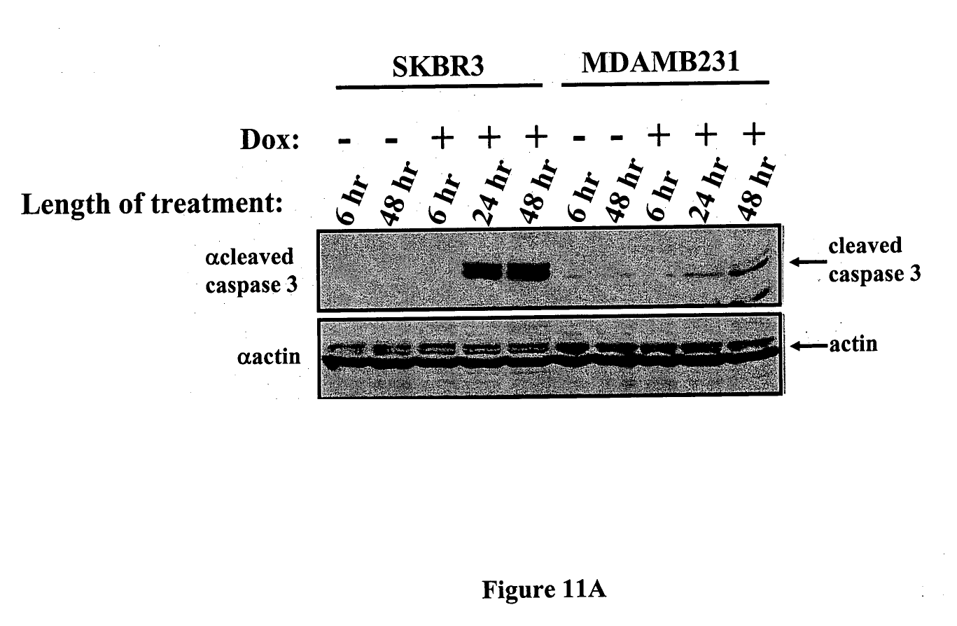

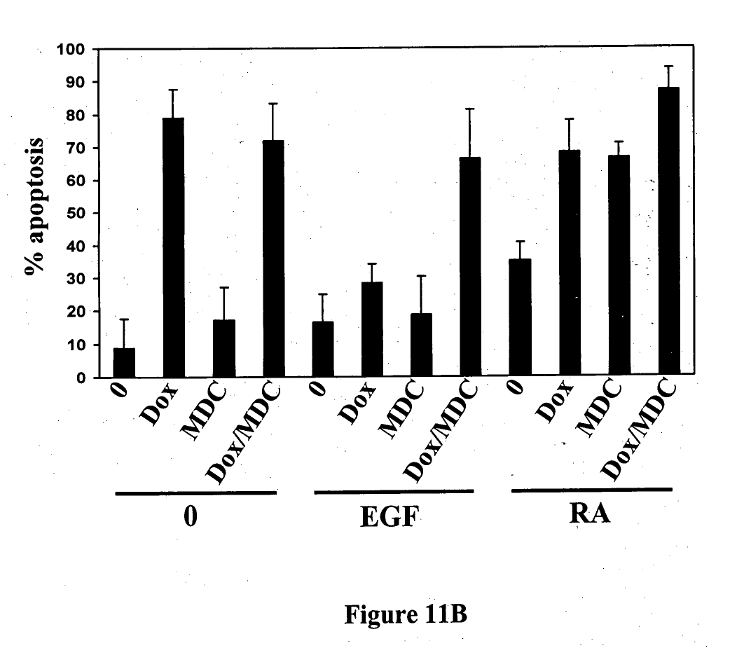

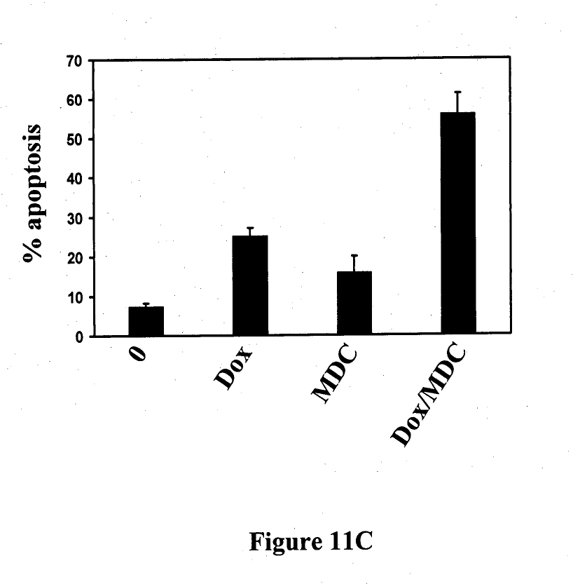

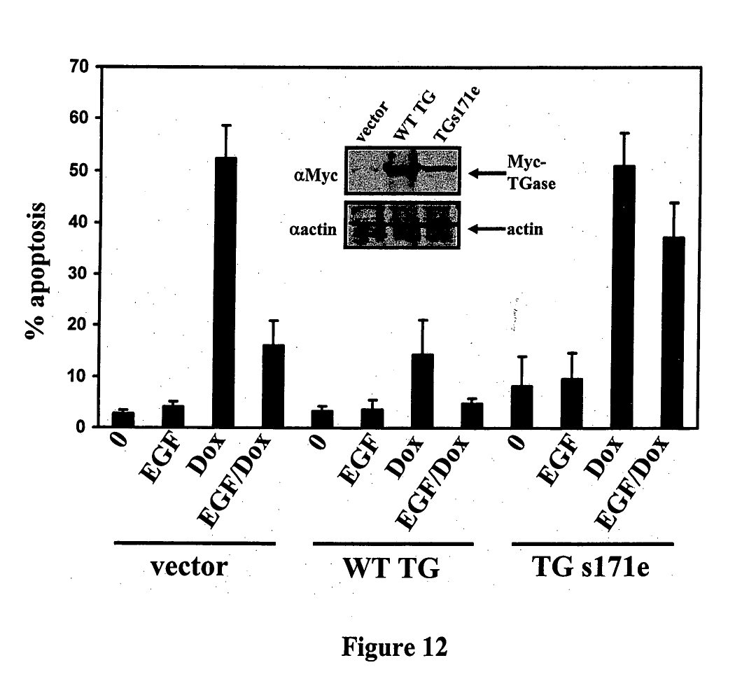

RA stimulation has been reported to up-regulate the activities of several signaling molecules, including the well established survival factor PI3 kinase. Recently, it was also shown that PI3 kinase activity was required for RA to increase TG expression in NIH3T3 cells, implicating PI3 kinase as a critical modulator of TGase expression in this cell type. Given that EGF and RA promote TGase expression in SKBR3 cells, and both of these stimuli are known activators of PI3 kinase, the question arose as to whether the induction of TGase expression by RA and EGF required PI3 kinase activity in SKBR3 cells. To examine this, a specific inhibitor of PI3 kinase known as LY294002 was utilized. Initially, the effectiveness of the inhibitor at blocking PI3 kinase activity was tested by comparing the amount of PI3 kinase activity present in cells incubated with or without LY294002 and then stimulated with EGF. Since the stimulation of AKT phosphorylation by EGF occurs in a PI3 kinase-dependent fashion, AKT phosphorylation was used as a convenient way to readout PI3 kinase activity. FIG. 10A shows that AKT phosphorylation was augmented in SKBR3 cells treated with EGF for 5 minutes. When the cells were pre-incubated with LY294002, EGF no longer stimulated AKT phosphorylation, suggesting that the inhibitor blocked PI3 kinase activity. It was then assessed whether PI3 kinase activity was important for the ability of RA or EGF to induce TG expression in the cancer cell line. LY294002 was nearly as effective at preventing RA-induced TG expression and GTP-binding ability in SKBR3 cells as it was in NIH3T3 cells (FIG. 10B; α-TGase and [α-³²P] GTP binding). Blocking PI3 kinase activation also compromised EGF-stimulated increases in TGase expression, implicating PI3 kinase activity as a common requirement for growth factor and retinoid-mediated TG expression in SKBR3 cells.

To further characterize the role of PI3 kinase in the regulation of TG expression in the cancer cell line, it was considered whether persistent PI3 kinase activity alone would induce the expression of TG. As shown in FIG. 10C, SKBR3 cells transiently transfected with a dominant-active form of PI3 kinase (a myristoylated form of the p110 catalytic subunit) did not exhibit increased TG protein levels. However, it was found that the induction of TG expression by EGF was significantly enhanced in cells overexpressing the dominant-active form of PI3 kinase. These findings indicate that, although not sufficient to induce TG expression on its own, constitutive PI3 kinase activity potentiated the induction of TG expression by EGF in SKBR3 cells.

### Example 17

### TG Activation Provides a Protective Effect from Doxorubicin-Mediated Apoptosis in SKBR3 and MDAMB231 Cells

The activation of TG has most often been implicated in the induction of apoptosis. On the other hand, recent studies have found TG activation to either not be directly involved with the programmed cell death process or to provide a protective effect from apoptotic-inducing stresses. It was investigated what role TG played in apoptosis in the SKBR3 and MDAMB231 cell lines. The apoptotic response of the cancer cell lines was assayed using the chemotherapeutic agent doxorubicin to induce cell death. As a means to detect cells undergoing apoptosis, immunoblot analysis with an antibody that specifically recognizes the cleaved or activated form of caspase 3 was performed on extracts of SKBR3 and MDAMB231 cells exposed to doxorubicin for increasing lengths of time. Following 24 hours of incubation with the chemotherapy, cleaved or activated caspase 3 was easily detected in SKBR3 (FIG. 11A; α-cleaved caspase 3). The cleaved caspase 3 levels persisted in these cells for at least another 24 hours, indicating that doxorubicin induced a potent and sustained activation of caspase 3 in SKBR3 cells. It was confirmed that these cells were undergoing apoptosis by evaluating another set of doxorubicin-treated SKBR3 cells for condensed and/or blebbed nuclei, a morphological feature unique to apoptotic cells. While control cells had low rates of apoptosis, nearly 80% of the cells exposed to doxorubicin displayed nuclear condensation or blebbing (FIG. 11B). The apoptotic response of MDAMB231 cells to doxorubicin was also determined. Rather than producing a strong cell death response as in SKBR3 cells, MDAMB231 cells were relatively resistant to the chemotherapy. MDAMB231 cells exposed to doxorubicin failed to induce the cleaved form of caspase 3 to the same extent as in SKBR3 cells (FIG. 11A) and showed an apoptotic rate of only 25% (FIG. 11C). Because chronic TG activation distinguishes MDAMB231 cells from SKBR3 cells (FIGS. 9A-B) and MDAMB231 cells are more resistant to doxorubicin-induced apoptosis than SKBR3 cells, it was possible that the constitutive TG activation contributed to the resistance of MDAMB231 cells from doxorubicin-mediated apoptosis. To address this possibility, the cell line was exposed to the chemotherapy in the presence of monodansylcadaverine (MDC), a competitive inhibitor of TG-catalyzed transamidation, and the resulting apoptotic rate was determined. As FIG. 11C shows, inhibiting TG activity with MDC rendered MDAMB231 cells slightly more than twice as susceptible to doxorubicin-induced apoptosis as compared to doxorubicin treatment alone.

Based on the data linking TG to a survival role in MDAMB231 cells, assessing whether TG had a similar function in SKBR3 cells was of particular interest. SKBR3 cells stimulated with EGF for 2 days prior to being exposed to the chemotherapy showed a 50% reduction in the rate of apoptosis of these cells (FIG. 11B). Since EGF enhances TG enzymatic activity in this cell line, it was then examined what effect inhibiting TG activation would have on the ability of EGF to inhibit doxorubicin-induced cell death. Incubating SKBR3 cells with MDC nearly completely eliminated the pro-survival effect of EGF from doxorubicin-induced apoptosis, implying that the protective effect afforded by EGF was dependent on the ability of the growth factor to up-regulate TG expression and activation. To further verify this result, a genetic approach was also employed. Similar to the findings obtained using MDC, expression of a dominant-negative form of TG (FIG. 12, inset; TG s171e) reduced the protective effect of EGF against doxorubicin-mediated apoptosis in SKBR3 cells (FIG. 12, graph). In contrast, overexpression of wildtype TG (FIG. 12, inset; WTTG) not only potentiated the survival role of EGF, but TG overexpression alone was sufficient to protect SKBR3 cells from doxorubicin-stimulated apoptosis (FIG. 12, graph). Therefore, in at least two different breast cancer cell lines, TG expression and activation elicited an anti-apoptotic effect from chemotherapeutic-induced cell death.

Although the invention has been described in detail for the purpose of illustration, it is understood that such detail is solely for that purpose, and variations can be made therein by those skilled in the art without departing from the spirit and scope of the invention which is defined by the following claims.

What is claimed:

1. A method of facilitating death of cancer cells in a subject, said method comprising:
inhibiting tissue transglutaminase in the subject under conditions effective to facilitate death of cancer cells.

2. The method according to claim 1, wherein said inhibiting is carried out by administering an inhibitor of tissue transglutaminase orally, intradermally, intramuscularly, intraperitoneally, intravenously, subcutaneously, or intranasally.

3. The method according to claim 1, wherein the tissue transglutaminase is human tissue transglutaminase.

4. The method according to claim 1, wherein said inhibiting is achieved with a compound which binds to one or more molecular surfaces of the tissue transglutaminase having a three dimensional crystal structure defined by the atomic coordinates set forth in FIG. 7.

5. The method according to claim 4, wherein the molecular surfaces of the tissue transglutaminase comprise atoms surrounding one or more of residues Lys-173, Phe-174, Arg-476, Arg-478, Val-479, Ser-482, Met-483, Arg-580, Leu-582, or Tyr-583.

6. A method of producing a tissue transglutaminase crystal suitable for X-ray diffraction comprising:
subjecting a solution of tissue transglutaminase under conditions effective to grow a crystal of tissue transglutaminase to a size suitable for X-ray diffraction; and
obtaining a tissue transglutaminase crystal suitable for X-ray diffraction.

7. The method of claim 6, wherein the crystal has space group P2₁2₁2₁ and unit cell dimensions of approximately a=132.479 Å, b=168.797 Å, and c=238.568 Å such that a three dimensional structure of the crystallized tissue transglutaminase can be determined to a resolution of about 2.8 Å or better.

8. The method of claim 6, wherein said subjecting is carried out by sitting drops using a vapor diffusion method.

9. A crystal produced by the method of claim 6.

10. A method for identifying candidate compounds suitable for facilitating death of cancer cells in a subject, said method comprising:
contacting tissue transglutaminase with a compound and
identifying those compounds which bind to the tissue transglutaminase as candidate compounds suitable for facilitating death of cancer cells in a subject.

11. The method according to claim 10, wherein the tissue transglutaminase is human tissue transglutaminase.

12. The method according to claim 10, wherein the compound binds to one or more molecular surfaces of the tissue transglutaminase, having a three dimensional crystal structure defined by the atomic coordinates set forth in FIG. 7.

13. The method according to claim 12, wherein the molecular surfaces of the tissue transglutaminase comprise atoms surrounding one or more of residues Lys-173, Phe-174, Arg-476, Arg-478, Val-479, Ser-482, Met-483, Arg-580, Leu-582, or Tyr-583.

14. A method for designing a compound suitable for facilitating death of cancer cells in a subject, said method comprising:
providing a three-dimensional structure of a crystallized tissue transglutaminase; and
designing a compound having a three-dimensional structure which will bind to one or more molecular surfaces of the tissue transglutaminase.

15. The method according to claim 14, wherein the tissue transglutaminase is human tissue transglutaminase.

16. The method according to claim 14, wherein the three dimensional structure of a crystallized tissue transglutaminase is defined by the atomic coordinates set forth in FIG. 7.

17. The method according to claim 16, wherein the molecular surfaces of the tissue transglutaminase comprise atoms surrounding one or more of residues Lys-173, Phe-174, Arg-476, Arg-478, Val-479, Ser-482, Met-483, Arg-580, Leu-582, or Tyr-583.

18. A compound designed by the method of claim 14.

19. A pharmaceutical composition comprising the compound of claim 18 and a pharmaceutical carrier.

20. A compound suitable for facilitating death of cancer cells in a subject, said compound having a three-dimensional structure which will bind to one or more molecular surfaces of the tissue transglutaminase having a three dimensional crystal structure defined by the atomic coordinates set forth in FIG. 7.

21. The compound according to claim 20, wherein the molecular surfaces of the tissue transglutaminase comprise atoms surrounding one or more of residues Lys-173, Phe-174, Arg-476, Arg-478, Val-479, Ser-482, Met-483, Arg-580, Leu-582, or Tyr-583.

22. A tissue transglutaminase crystal having a three dimensional crystal structure defined by the atomic coordinates set forth in FIG. 7.
